(19) United States
(12) Patent Application Publication
(10) Pub. No.: US 2024/0002820 A1
(43) Pub. Date: Jan. 4, 2024

(54) Polynucleotides, Compositions, and Methods for Genome Editing Involving Deamination

(71) Applicant: Intellia Therapeutics, Inc., Cambridge, MA (US)

(72) Inventors: Christian Dombrowski, Auburndale, MA (US); William Frederick Harrington, Cambridge, MA (US); Ruan Oliveira, Cambridge, MA (US)

(73) Assignee: Intellia Therapeutics, Inc., Cambridge, MA (US)

(21) Appl. No.: 18/332,335

(22) Filed: Jun. 9, 2023

Related U.S. Application Data
(63) Continuation of application No. PCT/US2021/062922, filed on Dec. 10, 2021.
(60) Provisional application No. 63/275,424, filed on Nov. 3, 2021.
(60) Provisional application No. 63/165,636, filed on Mar. 24, 2021.
(60) Provisional application No. 63/130,104, filed on Dec. 23, 2020.
(60) Provisional application No. 63/124,060, filed on Dec. 11, 2020.

(51) Int. Cl. C12N 9/22; C12N 9/78; C12N 15/10; C12N 15/63

(52) U.S. Cl. CPC C12N 9/22; C12N 9/78; C12N 2310/20; C12N 15/63; C12Y 305/04005; C12N 15/102

(57) ABSTRACT

Polynucleotides, polypeptides, compositions, and methods for genome editing using deamination are provided. An mRNA containing an open reading frame (ORF) encoding a polypeptide is provided herein. The polypeptide includes a cytidine deaminase and an RNA-guided nickase, and does not include a uracil glycosylase inhibitor (UGI). A composition provided herein may include two different mRNAs. The first mRNA includes an ORF encoding a cytidine deaminase and an RNA-guided nickase, and the second mRNA includes an ORF encoding uracil glycosylase inhibitor (UGI).

This application is a Continuation of International Application No. PCT/US2021/062922, filed Dec. 10, 2021, which claims the benefit under 35 U.S.C. 119(e) of U.S. Provisional Application No. 63/124,060, filed Dec. 11, 2020; U.S. Provisional Application No. 63/130,104, filed Dec. 23, 2020; U.S. Provisional Application No. 63/165,636, filed Mar. 24, 2021; and U.S. Provisional Application No. 63/275,424, filed Nov. 3, 2021, each of which is herein incorporated by reference in its entirety.

This application is filed with a Sequence Listing in electronic format. The Sequence Listing is provided as a file entitled “01155-0016-00US_ST26.XML” created on Jun. 9, 2023, which is 2,996,760 bytes in size. The information in the electronic format of the sequence listing is incorporated herein by reference in its entirety.

### INTRODUCTION AND SUMMARY

The present disclosure relates to polynucleotides, compositions, and methods for genomic editing involving deamination.

Base editing is a genome editing method that directly generates point mutations within a specific region of the genomic DNA without causing double-stranded breaks (DSB). DNA base editors (BEs) comprise fusions between a catalytically impaired Cas nuclease and a base-modification enzyme. Currently, effectors for cytidine-to-thymidine (C-to-T) editing fuse a cytidine deaminase with a nickase and a uracil glycosylase inhibitor (UGI). For example, base editor 3 (BE3) consists of a Cas9 nuclease bearing a mutation that converts it into a nickase (nCas9), fused to an APOBEC1 (apolipoprotein mRNA editing enzyme, catalytic polypeptide 1) deaminase and a UGI (e.g., Wang et al. Cell Research 27:1289-1292 (2017)), and it was reported that an “nCas9-fused UGI domain is still important for achieving high fidelity of base editing, even when high levels of free UGI is present.” As another example, an engineered human APOBEC3A (A3A or apolipoprotein mRNA editing enzyme, catalytic polypeptide 3A) deaminase has been investigated as a replacement for rat APOBEC1 deaminase (RAPO1) in the original BE3 (Gehrke et al., Nature Biotechnology, 36: 977-982 (2018)), but it was noted that the ability of base editors “to edit all Cs within their editing window can potentially have deleterious effects” and that “mutation of the N57 residue in the human A3A deaminase was critical to restoring its native target sequence precision in the context of a base editor and also to lowering its off-target editing activity.” Indeed, APOBEC3A-Class 2 Cas nickase (D10A) base editors have been reported as having a “high degree of mutagenicity” and as showing Cas9-independent off-target base editing (Doman et al., Nature Biotechnology 38:620-628 (2020)). Accordingly, improved compositions and methods for targeted C-to-T base editing using cytidine deaminases (e.g., an APOBEC3A deaminase) and RNA-guided nickase are needed.

Accordingly, the present disclosure provides polynucleotides, compositions, and methods for genomic editing involving a cytidine deaminase (e.g., an APOBEC3A deaminase) and an RNA-guided nickase that induce C-to-T conversions at target nucleotides with greater fidelity and may minimize bystander mutations. The present disclosure is based in part on the findings that by pairing a cytidine deaminase (e.g., an APOBEC deaminase) and an RNA-guided nickase system with UGI in trans (e.g., as a separate mRNA), it is possible to lower the amount of other base editing (C-to-A/G conversions, insertions, or deletions) and increase the purity of C-to-T editing.

Accordingly, the following embodiments are provided.

In some embodiments, a composition is provided, the composition comprising a first mRNA comprising a first open reading frame encoding a polypeptide comprising a cytidine deaminase and an RNA-guided nickase, and a second mRNA comprising a second open reading frame encoding a uracil glycosylase inhibitor (UGI), wherein the second mRNA is different from the first mRNA. In some embodiments, the first open reading frame does not comprise a sequence encoding a UGI. In some embodiments, the composition comprises a first composition and a second composition, wherein the first composition comprises a first mRNA comprising a first open reading frame encoding a polypeptide comprising a cytidine deaminase and an RNA-guided nickase and does not comprise a uracil glycosylase inhibitor (UGI), and the second composition comprises a second mRNA comprising a second open reading frame encoding a uracil glycosylase inhibitor (UGI), wherein the second mRNA is different from the first mRNA. In some embodiments, the composition comprises lipid nanoparticles.

In some embodiments, a method of modifying a target gene is provided, the method comprising delivering to a cell a first mRNA comprising a first open reading frame encoding a first polypeptide comprising a cytidine deaminase and an RNA-guided nickase, a second mRNA comprising a second open reading frame encoding a uracil glycosylase inhibitor (UGI), wherein the second mRNA is different from the first mRNA, and at least one guide RNA (gRNA).

In some embodiments, a method of modifying at least one cytidine within a target gene in a cell is provided, the method comprising expressing in the cell or contacting the cell with: (i) a first polypeptide comprising a cytidine deaminase and an RNA-guided nickase, wherein the first polypeptide does not comprise a uracil glycosylase inhibitor (UGI); (ii) a UGI polypeptide; and (iii) at least one guide RNA (gRNA) wherein the first polypeptide and gRNA form a complex with the target gene and modify the at least one cytidine in the target gene. In some embodiments, the ratio of the UGI polypeptide to the first polypeptide is from 10:1 to 50:1.

In some embodiments, an mRNA containing an open reading frame (ORF) encoding a polypeptide is provided, the polypeptide comprising a cytidine deaminase (e.g., an APOBEC3A deaminase (A3A)) and an RNA-guided nickase, wherein the polypeptide does not comprise a uracil glycosylase inhibitor (UGI). The polypeptide encoded by the mRNA is also provided. In some embodiments, a method of modifying a target gene is provided, the method comprising delivering an mRNA or a polypeptide described herein to a cell.

In some embodiments, a composition comprises two different mRNAs in which the first mRNA comprises an ORF encoding a cytidine deaminase (e.g., A3A) and an RNA-guided nickase, and the second mRNA comprises an ORF encoding uracil glycosylase inhibitor (UGI). In some embodiments, the first mRNA in the composition does not comprise an ORF encoding UGI. In some embodiments, the molar ratio of the second mRNA to the first mRNA is from 1:1 to 30:1, from 2:1 to 30:1, from 7:1 to 22:1. In some embodiments, the molar ratio of the second mRNA to the first mRNA is 22:1, 7:1, 2:1, or 1:1, 1:4, 1:11, or 1:33.

Further embodiments are provided throughout and described in the claims and Figures.

### BRIEF DESCRIPTION OF THE DRAWINGS

FIGS. 1A-1E show C-to-T conversion activity deaminase editors profiled against 5 different guide targeting sequences, respectively.

FIG. 2A shows the percentage of edited reads with the 4 target cytosines converted to thymidines for deaminase editors profiled using sg000296.

FIG. 2B shows the percentage of edited reads with the 5 target cytosines converted to thymidines for deaminase editors profiled using sg0001373.

FIG. 2C shows the percentage of edited reads with the 4 target cytosines converted to thymidines for deaminase editors profiled using sg001400.

FIG. 2D shows the percentage of edited reads with the 6 target cytosines converted to thymidines for deaminase editors profiled using sg003018.

FIG. 2E shows the percentage of edited reads with at least 6 of 8 target cytosines converted to thymidines for deaminase editors profiled using sg005883.

FIG. 3A-3E show the percentage of C-to-T conversion by base position for 5 different guide targeting sequences, respectively.

FIGS. 4A-4B show editing profile as a percentage of total reads in U-2OS cells (FIG. 4A) and HuH-7 cells (FIG. 4B).

FIGS. 5A-5B show editing profile as a percentage of edited reads in U-2OS cells (FIG. 5A) and HuH-7 cells (FIG. 5B).

FIGS. 6A-6B represent editing profile as a percentage of total reads upon titrating UGI mRNAs (SEQ ID NOs: 25 and 34).

FIG. 7 shows TTR editing levels in CD-1 mice treated with different LNP combinations with mRNA constructs and sgRNAs. FIG. 7 shows % editing of C-to-T conversions, C-to-A/G conversions, and indels of DNA sequences (NGS sequencing) extracted from liver tissue samples harvested from the CD-1 mice.

FIG. 8 shows TTR editing levels in liver tissue harvested from CD-1 mice treated with different LNP combinations with mRNA constructs and sgRNAs when the UGI sequence was delivered in trans (a separate mRNA).

FIG. 9A-9C show scatter plots representing statistically significant (p. adj.<0.05) differential gene expression events (black dots) in liver samples from mice treated with sgRNA G000282 and BC22n mRNA in the absence of UGI mRNA in trans (FIG. 9A), sgRNA G000282 and BC22n mRNA in the presence of UGI mRNA in trans (FIG. 9B) or Cas9 mRNA in the presence of UGI mRNA in trans (FIG. 9C).

FIG. 10 shows the editing profile in T cells following treatment with different mRNA constructs and CIITA-targeting sgRNAs.

FIG. 11 shows MHC class II negative cells assessed by flow cytometry analysis of T cells treated with different mRNA constructs and CIITA guide RNAs.

FIGS. 12A-12B show scatter plots showing statistically significant (*=p. adj.<0.05) differential gene expression events (black dots) in T cells treated with sgRNA G018076, UGI mRNA and either Cas9 mRNA (FIG. 12A) or BC22n mRNA (FIG. 12B).

FIGS. 13A-13B show scatter plots showing statistically significant (*=p. adj.<0.05) differential gene expression events (black dots) in T cells treated with sgRNA G018117, UGI mRNA and either Cas9 mRNA (FIG. 13A) or BC22n mRNA (FIG. 13B).

FIGS. 14A-14B show protein-protein interaction networks enriched among the list of differentially expressed genes in T cells treated with sgRNA G018076, UGI mRNA and either Cas9 mRNA (FIG. 14A) or BC22n mRNA (FIG. 14B).

FIGS. 15A-15B show protein-protein interaction networks enriched among the list of differentially expressed genes in T cells treated with sgRNA G018117, UGI mRNA and either Cas9 mRNA (FIG. 15A) or BC22 mRNA (FIG. 15B).

FIGS. 16A-16C show the editing profiles of T cells. Editing profiles in the on-target TRAC locus (FIG. 16A) in addition to 10 loci previously described as mutational hotspots in APOBEC-positive tumors (FIGS. 16B-16C).

FIGS. 17A-17E show the editing profiles of T cells when treated with varying levels of BC22n mRNA and Cas9 mRNAs. Cells were edited using sgRNAs G015995 (FIG. 17A), G016017 (FIG. 17B), G016206 (FIG. 17C), G018117 (FIG. 17D), or G016086 (FIG. 17E).

FIGS. 18A-18D show the editing profiles for T cells edited with four guides simultaneously using varying levels of BC22n mRNA or Cas9 mRNAs. The editing profile at each edited locus is represented separately: G015995 (FIG. 18A), G016017 (FIG. 18B), G016206 (FIG. 18C), G018117 (FIG. 18D).

FIGS. 19A-19I show phenotyping results as percent of cells negative for antibody binding with increasing total RNA for BC22n and Cas9 samples. FIG. 19A shows the percentage of B2M negative cells when B2M guide G015995 was used for editing. FIG. 19B shows the percentage of B2M negative cells when multiple guides were used for editing. FIG. 19C shows the percentage of CD3 negative cells when TRAC guide G016017 was used for editing. FIG. 19D shows the percentage of CD3 negative cells when TRBC guide G016206 was used for editing. FIG. 19E shows the percentage of CD3 negative cells when multiple guides were used for editing. FIG. 19F shows the percentage of MHC Class II negative cells when CIITA guide G018117 was used for editing. FIG. 19G shows the percentage of MHC Class II negative cells when multiple guides were used for editing. FIG. 19H shows the percentage of triple (B2M, CD3, MHC II) negative cells when multiple guides were used for editing. FIG. 19I shows the percentage of MHC class II negative T cells when CIITA guide G016086 was used for editing.

FIGS. 20A-20C show the editing profile for T cells while using varying levels of UGI mRNA in trans with various editor mRNAs. FIG. 20A shows the percent editing with BC22n mRNA at 27.3 nM. FIG. 20B shows the percent editing with BC22-2×UGI mRNA at 24.7 nM. FIG. 20C shows the percent editing with BE4Max mRNA at 24.0 nM.

FIG. 21 shows C-to-T conversion as a percentage of edited reads while using varying levels of UGI mRNA in trans with various editor mRNAs (BC22n at 27.3 nM, BC22-2×UGI at 24.7 nM, BE4Max at 24.0 nM).

FIG. 22 shows the mean percentage of T cells negative for cell surface expression of MHC II (% MHC II negative”) for several guides using Cas9 or BC22 in relation to the distance between the cut site boundary nucleotide shown as base pairs (“bp”). Positive numerical values indicate a splice site boundary nucleotide 3′ of the cut site, whereas the negative numerical values indicate a splice site boundary nucleotide 5′ of the cut site.

FIG. 23A shows an exemplary sgRNA (SEQ ID NO: 141, methylation not shown) in a possible secondary structure with labels designating individual nucleotides of the conserved region of the sgRNA, including the lower stem, bulge, upper stem, nexus (the nucleotides of which can be referred to as N1 through N18, respectively, in the 5′ to 3′ direction), and the hairpin region which includes hairpin 1 and hairpin 2 regions. A nucleotide between hairpin 1 and hairpin 2 is labeled n. A guide region may be present on an sgRNA and is indicated in this figure as “(N)x” preceding the conserved region of the sgRNA.

FIG. 23B labels the 10 conserved region YA sites in an exemplary sgRNA sequence (SEQ ID NO: 141, methylation not shown) from 1 to 10. The numbers 25, 45, 50, 56, 64, 67, and 83 indicate the position of the pyrimidine of YA sites 1, 5, 6, 7, 8, 9, and 10 in an sgRNA with a guide region indicated as (N)z, e.g., wherein x is optionally 20.

FIGS. 24A-B show results for efficiency of three CIITA guides (G016086, G016092, and G016067) for editing T cells with BC22. FIG. 24A shows the percent C-to-T conversion. FIG. 24B shows the percentage of MHC class II negative T cells.

FIG. 25 shows the percentage of B2M negative T cells after editing with different mRNA combinations. EP indicates electroporation.

FIG. 26 shows the percentage of single nucleotide variants (SNVs) that are C-to-U conversions in the transcriptome of T cells edited at B2M with different mRNA combinations (n.s.=not significant).

FIG. 27 shows the percentage of B2M negative T cells after-treatment with different mRNA combinations.

FIG. 28 shows the editing profiles at B2M locus in T cells after-treatment with different mRNA combinations.

FIG. 29 shows the percentage of SNVs that are C-to-T conversions in amplified genomic DNA from single T cells edited at B2M with different mRNA combinations (n.s.=not significant).

FIG. 30 shows the percentage of B2M negative eHap1 cells following editing with different mRNA combinations.

FIG. 31 shows the editing profiles of the B2M locus in eHap1 cells following treatment with different mRNA combinations.

FIG. 32 shows the percentage of SNVs that are C-to-T conversions in clonally expanded eHap1 cells editing at B2M with different mRNA combinations (n.s.=not statistically significant).

FIG. 33 shows the cell viability relative to untreated cells following electroporation or LNP delivery of BC22n or Cas9 editors and single or multiple guides.

FIG. 34 shows the total γH2AX spot intensity per nuclei following electroporation or LNP delivery of BC22n or Cas9 editors and single or multiple guides.

FIG. 35 shows the percentage editing at loci of interest following LNP delivery of BC22n or Cas9 editors and single or multiple guides.

FIG. 36 shows the percentage of negative cells for stated surface proteins following LNP delivery of BC22n or Cas9 editors and single or multiple guides.

FIG. 37 shows the percentage of interchromosomal translocations among total unique molecules following LNP delivery of BC22n or Cas9 editors and multiple guides.

FIG. 38 shows mean percent editing in mouse liver following treatment with various base editors.

FIG. 39 shows mean percent C-to-T conversion activity for base editor constructs designed with various deaminase domains.

FIGS. 40A-40K show percent of total reads containing at least 1 C to T conversion for base editing constructs designed with various linkers.

FIG. 41 shows mean percent editing at SERPINA1 in Huh-7 after treatment with base editor constructs designed using various linkers.

FIG. 42 shows EC90 for base editing mRNA designed with various linkers. The 95% confidence interval (CI) for each EC90 value is also shown.

FIG. 43 shows mean percent editing at the ANAPC5 locus in PHH using base editing constructs designed with various linkers.

FIG. 44 shows EC95 for base editing mRNA designed with various linkers. The 95% confidence interval (CI) for each EC95 value is also shown.

FIG. 45 shows mean percent editing at the TRAC locus in PHH using base editing constructs designed with various linkers.

FIG. 46 shows Mass of base editor mRNAs designed with various linkers that leads to 90% of the maximum (EC90) knockdown of CD3. The 95% confidence interval for each EC50 value is also shown.

FIG. 47 shows the mass of BC22n mRNAs designed with various linkers that leads to 90% of the maximum (EC90) knockdown of CD3, HLA-A3 and HLA-DR, DP, DQ (EC50). The 95% confidence interval (CI) for each EC50 value is also shown.

FIG. 48 shows mean percent editing at the TRAC locus in T cells treated with sgRNA in the 100-mer or 91-mer formats.

FIG. 49A shows mean percent editing at the TRBC1 locus in T cells treated with sgRNA in the 100-mer or 91-mer formats.

FIG. 49B shows mean percent editing at the TRBC2 locus in T cells treated with sgRNA in the 100-mer or 91-mer formats.

FIG. 50 shows mean percent editing at the CIITA locus in T cells treated with sgRNA in the 100-mer or 91-mer formats.

FIG. 51 shows mean percent editing at the B2M locus in T cells treated with sgRNA in the 100-mer or 91-mer formats.

FIG. 52 shows mean percent editing at the CD38 locus in T cells treated with sgRNA in the 100-mer or 91-mer formats.

FIG. 53A shows the mean percentage of CD8+ T cells that are negative for CD3 surface receptors following treatment with sgRNAs in the 100-mer or 91-mer formats targeting TRAC.

FIG. 53B shows the mean percentage of CD8+ T cells that are negative for CD3 surface receptors following treatment with sgRNAs in the 100-mer or 91-mer formats targeting TRBC.

FIG. 54A shows the mean percentage of CD8+ T cells that are negative for HLA-DR, DP, DQ surface receptors following treatment with sgRNAs in the 100-mer or 91-mer formats targeting CIITA.

FIG. 54B shows the mean percentage of CD8+ T cells that are negative for HLA-A surface receptors following treatment with sgRNAs in the 100-mer or 91-mer formats targeting HLA-A.

FIG. 55A shows the mean percentage of CD8+ T cells that are negative for B2M surface receptors following treatment with sgRNAs in the 100-mer or 91-mer formats targeting B2M.

FIG. 55B shows the mean percentage of CD8+ T cells that are negative for CD38 surface receptors following treatment with sgRNAs in the 100-mer or 91-mer formats targeting CD38.

FIG. 56 shows mean percent editing at the ANAPC5 locus in mouse liver with increasing amounts of UGI mRNA.

FIG. 57 shows percent C to T editing purity at the ANAPC5 locus in mouse liver following editing with increasing amounts of UGI mRNA.

FIG. 58A shows total editing at different BC22n-HiBiT mRNA concentrations at B2M in PHH cells.

FIG. 58B shows C-to-T purity at different UGI-HiBiT mRNA concentrations at B2M in PHH cells.

FIG. 58C shows total editing at different BC22-2×UGI-HiBiT mRNA concentrations at B2M in PHH cells.

FIG. 58D shows C-to-T purity at different BC22-2×UGI-HiBiT mRNA concentrations at B2M in PHH cells.

FIG. 59A shows total editing at different BC22n-HiBiT mRNA concentrations in T cells.

FIG. 59B shows C-to-T purity at different UGI-HiBiT mRNA concentrations in T cells.

FIG. 59C shows total editing at different BC22-2×UGI-HiBiT mRNA concentrations in T cells.

FIG. 59D shows C-to-T purity at different BC22-2×UGI-HiBiT mRNA concentrations in T cells.

FIG. 60 shows editing in liver tissue harvested from CD-1 mice treated with LNPs with fixed doses of sgRNA and base editor mRNA and different doses of UGI mRNA.

FIG. 61 shows C-to-T purity in liver tissue harvested from CD-1 mice treated with LNPs with fixed doses of sgRNA and base editor mRNA and different doses of UGI mRNA.

FIG. 62 shows the percent lysis of T cells targeted by NK cells at different effector:target (E:T) ratios treated with sgRNA and base editor and UGI mRNAs.

FIG. 63 shows conversion rate at each guide nucleotide position for high activity guides edited with a Spy base editor.

FIG. 64 shows conversion rate at each guide nucleotide position for high activity guides edited with an Nme2 base editor.

BRIEF DESCRIPTION OF DISCLOSED SEQUENCES

| SEQ ID | |
|---|---|
| NO | Description |
| 1 | mRNA encoding BC22n |
| 2 | Open reading frame for BC22n |
| 3 | Amino acid sequence for BC22n |
| 4 | mRNA encoding BC22n with HiBit tag |
| 5 | Open reading frame for BC22n with HiBit tag |
| 6 | Amino acid sequence for BC22n with HiBit tag |
| 7 | Not used |
| 8 | Open reading frame for Cas9 |
| 9 | Amino acid sequence for Cas9 |
| 10 | Not used |
| 11 | Open reading frame for Cas9 |
| 12 | Amino acid sequence for Cas9 |
| 13 | mRNA encoding BE3 |
| 14 | Open reading frame for BE3 |
| 15 | Amino acid sequence for BE3 |
| 16 | mRNA encoding BE3 |
| 17 | Open reading frame for BE3 |
| 18 | Amino acid sequence for BE3 |
| 19 | mRNA encoding BC22 |
| 20 | Open reading frame for BC22 |
| 21 | Amino acid sequence for BC22 |
| 22 | Not used |
| 23 | Open reading frame for Cas9 with HiBit tag |
| 24 | Amino acid sequence for Cas9 with HiBit tag |
| 25 | mRNA encoding UGI |
| 26 | Open reading frame for UGI |
| 27 | Amino acid sequence for UGI |
| 28 | mRNA encoding BC22 with 2x UGI |
| 29 | Open reading frame for BC22 with 2x UGI |
| 30 | Amino acid sequence for BC22 with 2x UGI |
| 31 | mRNA encoding BE4MAX protein |
| 32 | Open reading frame for BE4MAX protein |
| 33 | Amino acid sequence for BE4MAX protein |
| 34 | mRNA sequence encoding UGI |
| 35 | Open reading frame for UGI |
| 36 | Amino acid sequence for recombinant Cas9 |
| 37 | Not used |
| 38 | Not used |
| 39 | Not used |
| 40 | Amino acid sequence of H. sapiens APOBEC3A deaminase (A3A) |
| 41 | Amino acid sequence of R. norvegicus APOBEC1 |
| 42 | Exemplary coding sequence for UGI (SEQ ID NO. 43) |
| 43 | Amino acid sequence for exemplary UGI |
| 44 | Exemplary coding sequence for XTEN (SEQ ID NO. 46) |
| 45 | Exemplary coding sequence for XTEN (SEQ ID NO. 46) |
| 46 | Amino acid sequence for exemplary XTEN |
| 47 | Amino acid sequence for exemplary XTEN |
| 48 | Amino acid sequence for exemplary XTEN |
| 49 | Amino acid sequence for exemplary linker |
| 50 | Amino acid sequence for exemplary linker |
| 51 | Amino acid sequence for exemplary linker |
| 52 | Amino acid sequence for exemplary linker |
| 53 | Amino acid sequence for exemplary linker |
| 54 | Amino acid sequence for exemplary linker |
| 55 | Amino acid sequence for exemplary linker |
| 56 | Amino acid sequence for exemplary linker |
| 57 | Amino acid sequence for exemplary linker |
| 58 | Amino acid sequence for exemplary linker |
| 59 | Amino acid sequence for exemplary linker |
| 60 | Nucleic acid sequence for exemplary linker |
| 61 | Amino acid sequence for exemplary linker |
| 62 | Nucleic acid sequence for SV40 NLS |
| 63 | Amino acid sequence for SV40 NLS |
| 64 | pCI-Neo |
| 65 | Screening plasmid-invariant sequence |
| 66 | pUC19 |
| 67 | U6 promoter |
| 68 | CMV promoter |
| 69 | 3′ UTR from human albumin gene |
| 70 | Amino acid sequence of Spy Cas9 nickase (D10A) with 1x NLS as the C- |
| | terminal 7 amino acids |
| 71 | Spy Cas9 nickase (D10A) ORF encoding SEQ ID NO: 70 using minimal |
| | uridine codons as listed in Table 3, with start and stop codons |
| 72 | Spy Cas9 nickase (D10A) ORF coding sequence using minimal uridine codons |
| | as listed in Table 3 (no start or stop codons; suitable for inclusion in fusion |
| | protein coding sequence) |
| 73 | Amino acid sequence of Spy Cas9 nickase (without NLS) |
| 74 | Spy Cas9 nickase ORF encoding SEQ ID NO: 73 using minimal uridine |
| | codons as listed in Table 3, with start and stop codons |
| 75 | Spy Cas9 nickase coding sequence encoding SEQ ID NO: 73 using minimal |
| | uridine codons as listed in Table 3 (no start or stop codons; suitable for |
| | inclusion in fusion protein coding sequence) |
| 76 | Amino acid sequence of Spy Cas9 nickase with two nuclear localization |
| | signals as the C-terminal amino acids |
| 77 | Spy Cas9 nickase ORF encoding SEQ ID NO: 76 using minimal uridine |
| | codons as listed in Table 3, with start and stop codons |
| 78 | Spy Cas9 nickase coding sequence encoding SEQ ID NO: 76 using minimal |
| | uridine codons as listed in Table 3 (no start or stop codons; suitable for |
| | inclusion in fusion protein coding sequence) |
| 79 | Spy Cas9 nickase ORF using low A codons of Table 4, with start and stop codons |
| 80 | Spy Cas9 nickase ORF using low A codons of Table 4, with start and stop |
| | codons and no NLS |
| 81 | Spy Cas9 nickase ORF using low A codons of Table 4, with two C-terminal |
| | NLS sequences and start and stop codons |
| 82 | Spy Cas9 nickase ORF using low A/U codons of Table 4, with start and stop codons |
| 83 | Spy Cas9 nickase ORF using low A/U codons of Table 4, with two C-terminal |
| | NLS sequences and start and stop codons |
| 84 | Spy Cas9 nickase ORF using low A/U codons of Table 4, with start and stop |
| | codons and no NLS |
| 85 | Spy Cas9 nickase ORF using low A codons of Table 4 (no start or stop |
| | codons; suitable for inclusion in fusion protein coding sequence) |
| 86 | Spy Cas9 nickase ORF using low A codons of Table 4 (no NLS and no start or |
| | stop codons; suitable for inclusion in fusion protein coding sequence) |
| 87 | Spy Cas9 nickase ORF using low A codons of Table 4, with two C-terminal |
| | NLS sequences (no start or stop codons; suitable for inclusion in fusion protein |
| | coding sequence) |
| 88 | Spy Cas9 nickase ORF using low A/U codons of Table 4 (no start or stop |
| | codons; suitable for inclusion in fusion protein coding sequence) |
| 89 | Spy Cas9 nickase ORF using low A/U codons of Table 4, with two C-terminal |
| | NLS sequences (no start or stop codons; suitable for inclusion in fusion protein |
| | coding sequence) |
| 90 | Spy Cas9 nickase ORF using low A/U codons of Table 4 (no NLS and no start |
| | or stop codons; suitable for inclusion in fusion protein coding sequence) |
| 91 | Exemplary 5′ UTR |
| 92 | Exemplary 5′ UTR |
| 93 | Exemplary 5′ UTR |
| 94 | Exemplary 5′ UTR |
| 95 | Exemplary 5′ UTR |
| 96 | Exemplary 5′ UTR |
| 97 | Exemplary 5′ UTR |
| 98 | Exemplary 5′ UTR |
| 99 | Exemplary 3′ UTR |
| 100 | Exemplary 3′ UTR |
| 101 | Exemplary 3′ UTR |
| 102 | Exemplary 3′ UTR |
| 103 | Exemplary 3′ UTR |
| 104 | Exemplary 3′ UTR |
| 105 | Exemplary 3′ UTR |
| 106 | Exemplary 3′ UTR |
| 107 | Exemplary Kozak sequence |
| 108 | Exemplary Kozak sequence |
| 109 | Exemplary poly-A sequence |
| 110 | Exemplary NLS 1 |
| 111 | Exemplary NLS 2 |
| 112 | Exemplary NLS 3 |
| 113 | Exemplary NLS 4 |
| 114 | Exemplary NLS 5 |
| 115 | Exemplary NLS 6 |
| 116 | Exemplary NLS 7 |
| 117 | Exemplary NLS 8 |
| 118 | Exemplary NLS 9 |
| 119 | Exemplary NLS 10 |
| 120 | Exemplary NLS 11 |
| 121 | Alternative SV40 NLS |
| 122 | Nucleoplasmin NLS |
| 123 | Exemplary coding sequence for SV40 NLS |
| 124 | Exemplary coding sequence for NLS1 |
| 125 | Exemplary coding sequence for NLS2 |
| 126 | Exemplary coding sequence for NLS3 |
| 127 | Exemplary coding sequence for NLS4 |
| 128 | Exemplary coding sequence for NLS5 |
| 129 | Exemplary coding sequence for NLS6 |
| 130 | Exemplary coding sequence for NLS7 |
| 131 | Exemplary coding sequence for NLS8 |
| 132 | Exemplary coding sequence for NLS9 |
| 133 | Exemplary coding sequence for NLS10 |
| 134 | Exemplary coding sequence for NLS11 |
| 135 | Exemplary coding sequence for alternate SV40 NLS |
| 136-138 | Not used |
| 139 | Exemplary nucleotide sequence following the 3′ end of the guide sequence to |
| | form a crRNA |
| 140 | Conserved Portion of a spy Cas9 sgRNA |
| 141 | Modified sgRNA pattern, where N are nucleotides encoding a guide sequence |
| 142 | Exemplary guide constant region modification pattern (G282-C) |
| 143 | Exemplary guide modification pattern (G282-mN3Nx) |
| 144 | Exemplary guide modification pattern (G282-Nx) |
| 145 | Exemplary guide modification pattern (G282-N20) |
| 146-150 | Not used |
| 151 | Exemplary guide sequence |
| 152 | Exemplary guide sequence for B2M gene |
| 153 | Exemplary guide sequence for TTR gene |
| 154 | Exemplary guide sequence for TRAC gene |
| 155 | Exemplary guide sequence for TRBC1/2 gene |
| 156 | Exemplary guide sequence |
| 157 | Exemplary guide sequence for SERPINAl gene |
| 158 | Exemplary guide sequence for SERPINAl gene |
| 159 | Exemplary guide sequence |
| 160 | Exemplary guide sequence for CIITA gene |
| 161 | Exemplary guide sequence for CIITA gene |
| 162 | Exemplary guide sequence for CIITA gene |
| 163 | Exemplary guide sequence for CIITA gene |
| 164 | Exemplary guide sequence for CIITA gene |
| 165 | Exemplary guide sequence for CIITA gene |
| 166 | Exemplary guide sequence for CIITA gene |
| 167 | Exemplary guide sequence for CIITA gene |
| 168 | Exemplary guide sequence for CIITA gene |
| 169 | Exemplary guide sequence for CIITA gene |
| 170 | Exemplary guide sequence for CIITA gene |
| 171 | Exemplary guide sequence for CIITA gene |
| 172 | Exemplary guide sequence for CIITA gene |
| 173 | Exemplary guide sequence for CIITA gene |
| 174-176 | not used |
| 177 | G013009 guide RNA targeting TRAC |
| 178 | G016016 guide RNA targeting TRAC |
| 179 | G015991 guide RNA targeting B2M |
| 180 | G015996 guide RNA targeting B2M |
| 181 | G000297 guide RNA |
| 182 | G015995 guide RNA targeting B2M with guide sequence SEQ ID NO: 152 |
| 183 | G000282 guide RNA targeting TTR with guide sequence SEQ ID NO: 153 |
| 184 | G016017 guide RNA targeting TRAC with guide sequence SEQ ID NO: 154 |
| 185 | G016206 guide RNA targeting TRBC1/2 with guide sequence SEQ ID NO: 155 |
| 186 | SG000296 guide RNA |
| 187 | SG001373 guide RNA targeting SERPINA1 with guide sequence SEQ ID NO: 157 |
| 188 | SG001400 guide RNA targeting SERPINA1 with guide sequence SEQ ID NO: 158 |
| 189 | SG005883 guide RNA |
| 190 | SG003018 guide RNA targeting CIITA with guide sequence SEQ ID NO: 160 |
| 191 | G018075 guide RNA targeting CIITA with guide sequence SEQ ID NO: 161 |
| 192 | G018076 guide RNA targeting CIITA with guide sequence SEQ ID NO: 162 |
| 193 | G018077 guide RNA targeting CIITA with guide sequence SEQ ID NO: 163 |
| 194 | G018078 guide RNA targeting CIITA with guide sequence SEQ ID NO: 164 |
| 195 | G018081 guide RNA targeting CIITA with guide sequence SEQ ID NO: 165 |
| 196 | G018082 guide RNA targeting CIITA with guide sequence SEQ ID NO: 166 |
| 197 | G018084 guide RNA targeting CIITA with guide sequence SEQ ID NO: 167 |
| 198 | G018085 guide RNA targeting CIITA with guide sequence SEQ ID NO: 168 |
| 199 | G018091 guide RNA targeting CIITA with guide sequence SEQ ID NO: 169 |
| 200 | G018100 guide RNA targeting CIITA with guide sequence SEQ ID NO: 170 |
| 201 | G018117 guide RNA targeting CIITA with guide sequence SEQ ID NO: 171 |
| 202 | G018118 guide RNA targeting CIITA with guide sequence SEQ ID NO: 172 |
| 203 | G018120 guide RNA targeting CIITA with guide sequence SEQ ID NO: 173 |
| 204-210 | Not used |
| 211 | Amino acid sequence for exemplary linker |
| 212 | Amino acid sequence for exemplary linker |
| 213 | Amino acid sequence for exemplary linker |
| 214 | Amino acid sequence for exemplary linker |
| 215 | Amino acid sequence for exemplary linker |
| 216 | Amino acid sequence for exemplary linker |
| 217 | Amino acid sequence for exemplary linker |
| 218 | Amino acid sequence for exemplary linker |
| 219 | Amino acid sequence for exemplary linker |
| 220 | Amino acid sequence for exemplary linker |
| 221 | Amino acid sequence for exemplary linker |
| 222 | Amino acid sequence for exemplary linker |
| 223 | Amino acid sequence for exemplary linker |
| 224 | Amino acid sequence for exemplary linker |
| 225 | Amino acid sequence for exemplary linker |
| 226 | Amino acid sequence for exemplary linker |
| 227 | Amino acid sequence for exemplary linker |
| 228 | Amino acid sequence for exemplary linker |
| 229 | Amino acid sequence for exemplary linker |
| 230 | Amino acid sequence for exemplary linker |
| 231 | Amino acid sequence for exemplary linker |
| 232 | Amino acid sequence for exemplary linker |
| 233 | Amino acid sequence for exemplary linker |
| 234 | Amino acid sequence for exemplary linker |
| 235 | Amino acid sequence for exemplary linker |
| 236 | Amino acid sequence for exemplary linker |
| 237 | Amino acid sequence for exemplary linker |
| 238 | Amino acid sequence for exemplary linker |
| 239 | Amino acid sequence for exemplary linker |
| 240 | Amino acid sequence for exemplary linker |
| 241 | Amino acid sequence for exemplary linker |
| 242 | Amino acid sequence for exemplary linker |
| 243 | Amino acid sequence for exemplary linker |
| 244 | Amino acid sequence for exemplary linker |
| 245 | Amino acid sequence for exemplary linker |
| 246 | Amino acid sequence for exemplary linker |
| 247 | Amino acid sequence for exemplary linker |
| 248 | Amino acid sequence for exemplary linker |
| 249 | Amino acid sequence for exemplary linker |
| 250 | Amino acid sequence for exemplary linker |
| 251 | Amino acid sequence for exemplary linker |
| 252 | Amino acid sequence for exemplary linker |
| 253 | Amino acid sequence for exemplary linker |
| 254 | Amino acid sequence for exemplary linker |
| 255 | Amino acid sequence for exemplary linker |
| 256 | Amino acid sequence for exemplary linker |
| 257 | Amino acid sequence for exemplary linker |
| 258 | Amino acid sequence for exemplary linker |
| 259 | Amino acid sequence for exemplary linker |
| 260 | Amino acid sequence for exemplary linker |
| 261 | Amino acid sequence for exemplary linker |
| 262 | Amino acid sequence for exemplary linker |
| 263 | Amino acid sequence for exemplary linker |
| 264 | Amino acid sequence for exemplary linker |
| 265 | Amino acid sequence for exemplary linker |
| 266 | Amino acid sequence for exemplary linker |
| 267 | Amino acid sequence for exemplary linker |
| 268 | Amino acid sequence for exemplary linker |
| 269 | Amino acid sequence for exemplary linker |
| 270 | Amino acid sequence for exemplary linker |
| 271 | Amino acid sequence for exemplary linker |
| 272 | Amino acid sequence for exemplary linker |
| 273-300 | Not Used |
| 301 | Exemplary mRNA encoding APOBEC3A-Nme2D16A |
| 302 | Exemplary open reading frame for APOBEC3A-Nme2D16A |
| 303 | Exemplary amino acid sequence for APOBEC3A-Nme2D16A |
| 304 | Exemplary mRNA encoding APOBEC3A-Nme2D16A |
| 305 | Exemplary open reading frame for APOBEC3A-Nme2D16A |
| 306 | Exemplary amino acid sequence for APOBEC3A-Nme2D16A |
| 307 | Exemplary mRNA encoding APOBEC3A-Nme2D16A |
| 308 | Exemplary open reading frame for APOBEC3A-Nme2D16A |
| 309 | Exemplary amino acid sequence for APOBEC3A-Nme2D16A |
| 310 | Exemplary mRNA encoding APOBEC3A-Nme2D16A |
| 311 | Exemplary open reading frame for APOBEC3A-Nme2D16A |
| 312 | Exemplary amino acid sequence for APOBEC3A-Nme2D16A |
| 313 | Exemplary amino acid sequence for NLS-NLS-APOBEC3A-L070-Nme2D16A |
| 314 | mRNA encoding BC22-2XUGI with a C-terminal HiBiT tag (BC22-2XUGI-HibIT) |
| 315 | mRNA encoding BC22-Nme2D16A (Nme2 BC22n) |
| 316 | mRNA encoding UGI with a C-terminal HiBiT tag (UGI-HiBiT) |
| 317-319 | Not Used |
| 320 | Amino acid sequence for Nme2Cas9 |
| 321-337 | Exemplary amino acid sequences for base editor with linker |
| 340 | mRNA encoding Nme2Cas9 |
| 341-357 | Exemplary mRNA sequences for base editor with linker |
| 360 | Open reading frame encoding Nme2Cas9 |
| 361-377 | Exemplary open reading frame sequences for base editor with linker |
| 378-386 | Not Used |
| 387 | Exemplary amino acid sequence for D16A Nme2Cas9 nickase |
| 388 | Exemplary coding sequence for D16A Nme2Cas9 nickase |
| 389 | Exemplary coding sequence for D16A Nme2Cas9 nickase |
| 390 | Exemplary coding sequence for D16A Nme2Cas9 nickase |
| 391 | Exemplary open reading frame for D16A Nme2Cas9 nickase |
| 392 | Exemplary open reading frame for D16A Nme2Cas9 nickase |
| 393 | Exemplary open reading frame for D16A Nme2Cas9 nickase |
| 394-400 | Not Used |
| 401-416 | Exemplary guide RNAs targeting HLA-A |
| 417 | Not Used |
| 418-422 | Exemplary guide RNAs targeting HLA-A |
| 423 | Not Used |
| 424 | Exemplary guide RNAs targeting HLA-A |
| 425-426 | Not Used |
| 429-435 | Exemplary guide RNAs targeting HLA-A |
| 436 | Not Used |
| 437-443 | Exemplary guide RNAs targeting HLA-A |
| 444 | Not Used |
| 445-453 | Exemplary guide RNAs targeting HLA-A |
| 454 | Not Used |
| 455-495 | Exemplary guide RNAs targeting HLA-A |
| 496 | G023522 Exemplary guide RNA Targeting CD38 gene |
| 497 | G019427 Exemplary guide RNA Targeting ANAPC5 |
| 498 | G023519 Exemplary guide RNA Targeting B2M |
| 499 | G023520 Exemplary guide RNA Targeting TRAC |
| 500 | G023521 Exemplary guide RNA Targeting CIITA |
| 501 | G023523 Exemplary guide RNA Targeting HLA-A |
| 502 | G023524 Exemplary guide RNA Targeting TRBC1/2 |
| 503 | G020073 Exemplary Nme2Cas9 guide RNA |
| 504 | G020927 Exemplary Nme2Cas9 guide RNA |
| 505 | G020928 Exemplary Nme2Cas9 guide RNA |
| 506 | G020929 Exemplary Nme2Cas9 guide RNA |
| 507 | G021237 Exemplary Nme2Cas9 guide RNA |
| 508 | G021249 Exemplary Nme2Cas9 guide RNA |
| 509 | G021321 Exemplary Nme2Cas9 guide RNA |
| 510 | G021844 Exemplary Nme2Cas9 guide RNA with non-peptide linker |
| 511 | G000502 Exemplary guide RNA targeting TTR |
| 512 | Exemplary NmeCas9 sgRNA |
| 513 | Conserved region of exemplary shortened NmeCas9 sgRNA |
| 514 | Conserved region of Exemplary shortened NmeCas9 sgRNA pattern |
| 515 | Conserved region of Exemplary shortened NmeCas9 sgRNA pattern |
| 516 | Conserved region of Exemplary shortened/modified NmeCas9 sgRNA pattern |
| | (Mod-77) |
| 517 | Conserved region of Exemplary shortened/modified NmeCas9 sgRNA |
| | comprising linkers (Mod-78) |
| 518 | Exemplary shortened NmeCas9 sgRNA comprising linkers |
| 519 | Exemplary shortened/modified NmeCas9 guide RNA comprising linkers |
| 520 | Exemplary shortened/modified SpyCas9 guide RNA |
| 521 | Exemplary shortened SpyCas9 guide RNA |
| 522 | Exemplary shortened/modified NmeCas9 guide RNA |
| 523 | Exemplary shortened/modified SpyCas9 guide RNA comprising linkers |
| 524 | Exemplary shortened SpyCas9 guide RNA comprising linkers |
| 525-528 | Not Used |
| 529 | G016788 Exemplary guide RNA |
| 530-617 | Exemplary guide RNAs targeting CIITA |
| 618-705 | Exemplary guide sequence for TRBC gene |
| 706-746 | Exemplary guide sequence for TRAC gene |
| 747-762 | Exemplary guide RNA sequence for TRAC gene |
| 763-800 | Not Used |
| 801-852 | Exemplary guide RNA sequences for TRBC gene |
| 853-868 | Exemplary guide RNA sequences for TRAC gene |
| 869-956 | Exemplary guide RNA for CD38 gene |
| 957-959 | Not used |
| 960-1023 | Exemplary amino acid sequences of cytidine deaminases |
| 1024-1075 | Exemplary guide RNA sequences for TRBC gene |
| 1076-1163 | Exemplary guide RNA sequences for CIITA gene |

See the Sequence Table below for the sequences themselves. Transcript sequences may generally include GGG as the first three nucleotides for use with ARCA or AGG as the first three nucleotides for use with CleanCap™. Accordingly, the first three nucleotides can be modified for use with other capping approaches, such as Vaccinia capping enzyme. Promoters and poly-A sequences are not included in the transcript sequences. A promoter such as a U6 promoter (SEQ ID NO: 67) or a CMV Promotor (SEQ ID NO: 68) and a poly-A sequence such as SEQ ID NO: 109 can be appended to the disclosed transcript sequences at the 5′ and 3′ ends, respectively. Most nucleotide sequences are provided as DNA but can be readily converted to RNA by changing Ts to Us.

### DETAILED DESCRIPTION OF CERTAIN EMBODIMENTS

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents, which may be included within the invention as defined by the appended claims.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

The term “about” or “approximately” means an acceptable error for a particular value as determined by one of ordinary skill in the art, which depends in part on how the value is measured or determined, or a degree of variation that does not substantially affect the properties of the described subject matter, or within the tolerances accepted in the art, e.g., within 10%, 5%, 2%, or 1%. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The section headings used herein are for organizational purposes only and are not to be construed as limiting the desired subject matter in any way. In the event that any literature incorporated by reference contradicts the express content of this specification, including but not limited to a definition, the express content of this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### I. Definitions

Unless stated otherwise, the following terms and phrases as used herein are intended to have the following meanings:

The term “or combinations thereof” as used herein refers to all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, CBBA, CABA, and so forth. The skilled artisan will understand that typically there is no limit on the number of items or terms in any combination, unless otherwise apparent from the context.

As used herein, the term “kit” refers to a packaged set of related components, such as one or more polynucleotides or compositions and one or more related materials such as delivery devices (e.g., syringes), solvents, solutions, buffers, instructions, or desiccants.

“Or” is used in the inclusive sense, i.e., equivalent to “and/or,” unless the context requires otherwise.

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N4-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA.

“Polypeptide” as used herein refers to a multimeric compound comprising amino acid residues that can adopt a three-dimensional conformation. Polypeptides include but are not limited to enzymes, enzyme precursor proteins, regulatory proteins, structural proteins, receptors, nucleic acid binding proteins, antibodies, etc. Polypeptides may, but do not necessarily, comprise post-translational modifications, non-natural amino acids, prosthetic groups, and the like.

As used herein, a “cytidine deaminase” means a polypeptide or complex of polypeptides that is capable of cytidine deaminase activity, that is catalyzing the hydrolytic deamination of cytidine or deoxycytidine, typically resulting in uridine or deoxyuridine. Cytidine deaminases encompass enzymes in the cytidine deaminase superfamily, and in particular, enzymes of the APOBEC family (APOBEC1, APOBEC2, APOBEC4, and APOBEC3 subgroups of enzymes), activation-induced cytidine deaminase (AID or AICDA) and CMP deaminases (see, e.g., Conticello et al., Mol. Biol. Evol. 22:367-77, 2005; Conticello, Genome Biol. 9:229, 2008; Muramatsu et al., J. Biol. Chem. 274: 18470-6, 1999); Carrington et al., Cells 9:1690 (2020)). In some embodiments, variants of any known cytidine deaminase or APOBEC protein are encompassed. Variants include proteins having a sequence that differs from wild-type protein by one or several mutations (i.e., substitutions, deletions, insertions), such as one or several single point substitutions. For instance, a shortened sequence could be used, e.g., by deleting N-terminal, C-terminal, or internal amino acids, preferably one to four amino acids at the C-terminus of the sequence. As used herein, the term “variant” refers to allelic variants, splicing variants, and natural or artificial mutants, which are homologous to a reference sequence. The variant is “functional” in that it shows a catalytic activity of DNA editing.

As used herein, the term “APOBEC3A” refers to a cytidine deaminase such as the protein expressed by the human A3A gene. The APOBEC3A may have catalytic DNA editing activity. An amino acid sequence of APOBEC3A has been described (UniPROT accession ID: p31941) and is included herein as SEQ ID NO: 40. In some embodiments, the APOBEC3A protein is a human APOBEC3A protein and/or a wild-type protein. Variants include proteins having a sequence that differs from wild-type APOBEC3A protein by one or several mutations (i.e., substitutions, deletions, insertions), such as one or several single point substitutions. For instance, a shortened APOBEC3A sequence could be used, e.g. by deleting N-terminal, C-terminal, or internal amino acids, preferably one to four amino acids at the C-terminus of the sequence. As used herein, the term “variant” refers to allelic variants, splicing variants, and natural or artificial mutants, which are homologous to an APOBEC3A reference sequence. The variant is “functional” in that it shows a catalytic activity of DNA editing. In some embodiments, an APOBEC3A (such as a human APOBEC3A) has a wild-type amino acid position 57 (as numbered in the wild-type sequence). In some embodiments, an APOBEC3A (such as a human APOBEC3A) has an asparagine at amino acid position 57 (as numbered in the wild-type sequence).

As used herein, a “nickase” is an enzyme that creates a single-strand break (also known as a “nick”) in double strand DNA, i.e., cuts one strand but not the other of the DNA double helix. As used herein, an “RNA-guided nickase” means a polypeptide or complex of polypeptides having DNA nickase activity, wherein the DNA nickase activity is sequence-specific and depends on the sequence of the RNA. Exemplary RNA-guided nickases include Cas nickases. Cas nickases include, but are not limited to, nickase forms of a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. Class 2 Cas nickases include Class 2 Cas nuclease variants in which only one of the two catalytic domains is inactivated, which have RNA-guided DNA nickase activity. Class 2 Cas nickases include, for example, Cas9 (e.g., H840A, DOA, or N863A variants of SpyCas9), Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g., K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like protein domain. Cpf1 sequences of Zetsche are incorporated by reference in their entirety. See, e.g., Zetsche, Tables S1 and S3. “Cas9” encompasses S. pyogenes (Spy) Cas9, the variants of Cas9 listed herein, and equivalents thereof. See, e.g., Makarova et al., Nat Rev Microbiol, 13(11): 722-36 (2015); Shmakov et al., Molecular Cell. 60:385-397 (2015).

Several Cas9 orthologs have been obtained from N. meningitidis (Esvelt et al., NAT. METHODS, vol. 10, 2013, 1116-1121; Hou et al., PNAS, vol. 110, 2013, pages 15644-15649; Edraki et al., Mol. Cell 73:714-726, 2019) (Nme1Cas9, Nme2Cas9, and Nme3Cas9). The Nme2Cas9 ortholog functions efficiently in mammalian cells, recognizes an N4CC PAM, and can be used for in vivo editing (Ran et al., NATURE, vol. 520, 2015, pages 186-191; Kim et al., NAT. COMMUN., vol. 8, 2017, pages 14500). Nme2Cas9 has been shown to be naturally resistant to off-target editing (Lee et al., MOL. THER., vol. 24, 2016, pages 645-654; Kim et al., 2017). See also e.g., WO/2020081568 (e.g., pages 28 and 42), describing an Nme2Cas9 D16A nickase, the contents of which are hereby incorporated by reference in its entirety. Throughout, “NmeCas9” is generic and an encompasses any type of NmeCas9, including, Nme1Cas9, Nme2Cas9, and Nme3Cas9.

As used herein, the term “fusion protein” refers to a hybrid polypeptide which comprises polypeptides from at least two different proteins or sources. One polypeptide may be located at the amino-terminal (N-terminal) portion of the fusion protein or at the carboxy-terminal (C-terminal) protein thus forming an “amino-terminal fusion protein” or a “carboxy-terminal fusion protein,” respectively. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

The term “linker,” as used herein, refers to a chemical group or a molecule linking two adjacent molecules or moieties. Typically, the linker is positioned between, or flanked by, two groups, molecules, or other moieties and connected to each one via a covalent bond. In some embodiments, the linker is an amino acid or a plurality of amino acids (e.g., a peptide or protein) such as a 16-amino acid residue “XTEN” linker, or a variant thereof (See, e.g., the Examples; and Schellenberger et al. A recombinant polypeptide extends the in vivo half-life of peptides and proteins in a tunable manner. Nat. Biotechnol. 27, 1186-1190 (2009)). In some embodiments, the XTEN linker comprises the sequence SGSETPGTSESATPES (SEQ ID NO: 46), SGSETPGTSESA (SEQ ID NO: 47), or SGSETPGTSESATPEGGSGGS (SEQ ID NO: 48). In some embodiments, the linker comprises one or more sequences selected from SEQ ID NOs: 46-59, 61 and 211-272.

As used herein, the term “uracil glycosylase inhibitor”, “uracil-DNA glycosylase inhibitor” or “UGI” refers to a protein that is capable of inhibiting a uracil-DNA glycosylase (UDG) base-excision repair enzyme (e.g., UniPROT ID: P14739; SEQ ID NO: 27; SEQ ID NO:43).

As used herein, “open reading frame” or “ORF” of a gene refers to a sequence consisting of a series of codons that specify the amino acid sequence of the protein that the gene codes for. The ORF generally begins with a start codon (e.g., ATG in DNA or AUG in RNA) and ends with a stop codon, e.g., TAA, TAG or TGA in DNA or UAA, UAG, or UGA in RNA.

“Guide RNA”, “gRNA”, and “guide” are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “gRNA” refers to each type. The trRNA may be a naturally-occurring sequence, or a trRNA sequence with modifications or variations compared to naturally-occurring sequences.

As used herein, a “guide sequence” or “guide region” or “spacer” or “spacer sequence” and the like refers to a sequence within a gRNA that is complementary to a target sequence and functions to direct a gRNA to a target sequence for binding or modification (e.g., cleavage) by an RNA-guided nickase. A guide sequence can be 20 nucleotides in length, e.g., in the case of Streptococcus pyogenes (i.e., Spy Cas9 (also referred to as SpCas9)) and related Cas9 homologs/orthologs. Shorter or longer sequences can also be used as guides, e.g., 15-, 16-, 17-, 18-, 19-, 21-, 22-, 23-, 24-, or 25-nucleotides in length. A guide sequence can be 20-25 nucleotides in length, e.g., in the case of Nme Cas9, e.g., 20-, 21-, 22-, 23-, 24- or 25-nucleotides in length. For example, a guide sequence of 24 nucleotides in length can be used with Nme Cas9, e.g., Nme2 Cas9.

In some embodiments, the target sequence is in a gene or on a chromosome, for example, and is complementary to the guide sequence. In some embodiments, the degree of complementarity or identity between a guide sequence and its corresponding target sequence may be about 75%, 80%, 85%, 90%, 95%, or 100%. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides.

As used herein, a “target sequence” or “genomic target sequence” refers to a sequence of nucleic acid in a target gene that has complementarity to the guide sequence of the gRNA. The interaction of the target sequence and the guide sequence directs an RNA-guided DNA binding agent to bind, and potentially nick or cleave (depending on the activity of the agent), within the target sequence. Target sequences for Cas proteins include both the positive and negative strands of genomic DNA (i.e., the sequence given and the sequence's reverse compliment), as a nucleic acid substrate for a Cas protein is a double stranded nucleic acid. Accordingly, where a guide sequence is said to be “complementary to a target sequence,” it is to be understood that the guide sequence may direct an RNA-guided DNA binding agent (e.g., dCas9 or impaired Cas9) to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server are generally appropriate.

“mRNA” is used herein to refer to a polynucleotide that is not DNA and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof. In general, mRNAs do not contain a substantial quantity of thymidine residues (e.g., 0 residues or fewer than 30, 20, 10, 5, 4, 3, or 2 thymidine residues; or less than 10%, 9%, 8%, 7%, 6%, 5%, 4%, 4%, 3%, 2%, 1%, 0.5%, 0.2%, or 0.1% thymidine content). An mRNA can contain modified uridines at some or all of its uridine positions.

“Modified uridine” is used herein to refer to a nucleoside other than thymidine with the same hydrogen bond acceptors as uridine and one or more structural differences from uridine. In some embodiments, a modified uridine is a substituted uridine, i.e., a uridine in which one or more non-proton substituents (e.g., alkoxy, such as methoxy) takes the place of a proton. In some embodiments, a modified uridine is pseudouridine. In some embodiments, a modified uridine is a substituted pseudouridine, i.e., a pseudouridine in which one or more non-proton substituents (e.g., alkyl, such as methyl) takes the place of a proton. In some embodiments, a modified uridine is any of a substituted uridine, pseudouridine, or a substituted pseudouridine.

“Uridine position” as used herein refers to a position in a polynucleotide occupied by a uridine or a modified uridine. Thus, for example, a polynucleotide in which “100% of the uridine positions are modified uridines” contains a modified uridine at every position that would be a uridine in a conventional RNA (where all bases are standard A, U, C, or G bases) of the same sequence. Unless otherwise indicated, a U in a polynucleotide sequence of a sequence table or sequence listing in or accompanying this disclosure can be a uridine or a modified uridine.

As used herein, the “minimal uridine codon(s)” for a given amino acid is the codon(s) with the fewest uridines (usually 0 or 1 except for a codon for phenylalanine, where the minimal uridine codon has 2 uridines). Modified uridine residues are considered equivalent to uridines for the purpose of evaluating uridine content.

As used herein, the “uridine dinucleotide (UU) content” of an ORF can be expressed in absolute terms as the enumeration of UU dinucleotides in an ORF or on a rate basis as the percentage of positions occupied by the uridines of uridine dinucleotides (for example, AUUAU would have a uridine dinucleotide content of 40% because 2 of 5 positions are occupied by the uridines of a uridine dinucleotide). Modified uridine residues are considered equivalent to uridines for the purpose of evaluating uridine dinucleotide content.

As used herein, the “minimal adenine codon(s)” for a given amino acid is the codon(s) with the fewest adenines (usually 0 or 1 except for a codon for lysine and asparagine, where the minimal adenine codon has 2 adenines). Modified adenine residues are considered equivalent to adenines for the purpose of evaluating adenine content.

As used herein, the “adenine dinucleotide content” of an ORF can be expressed in absolute terms as the enumeration of AA dinucleotides in an ORF or on a rate basis as the percentage of positions occupied by the adenines of adenine dinucleotides (for example, UAAUA would have an adenine dinucleotide content of 40% because 2 of 5 positions are occupied by the adenines of an adenine dinucleotide). Modified adenine residues are considered equivalent to adenines for the purpose of evaluating adenine dinucleotide content.

As used herein, “indels” refer to insertion/deletion mutations consisting of a number of nucleotides that are either inserted or deleted, e.g., at the site of double-stranded breaks (DSBs) in a target nucleic acid.

As used herein, “knockdown” refers to a decrease in expression of a particular gene product (e.g., protein, mRNA, or both). Knockdown of a protein can be measured either by detecting protein secreted by tissue or population of cells (e.g., in serum or cell media) or by detecting total cellular amount of the protein from a tissue or cell population of interest. Methods for measuring knockdown of mRNA are known and include sequencing of mRNA isolated from a tissue or cell population of interest. In some embodiments, “knockdown” may refer to some loss of expression of a particular gene product, for example a decrease in the amount of mRNA transcribed or a decrease in the amount of protein expressed or secreted by a population of cells (including in vivo populations such as those found in tissues).

As used herein, “knockout” refers to a loss of expression of a particular protein in a cell. Knockout can be measured either by detecting the amount of protein secretion from a tissue or population of cells (e.g., in serum or cell media) or by detecting total cellular amount of a protein a tissue or a population of cells. In some embodiments, the methods of the disclosure “knockout” a target protein one or more cells (e.g., in a population of cells including in vivo populations such as those found in tissues). In some embodiments, a knockout is not the formation of mutant of the target protein, for example, created by indels, but rather the complete loss of expression of the target protein in a cell, i.e., decrease of expression to below the level of detection of the assay used.

As used herein, the terms “nuclear localization signal” (NLS) or “nuclear localization sequence” refers to an amino acid sequence which induces transport of molecules comprising such sequences or linked to such sequences into the nucleus of eukaryotic cells. The nuclear localization signal may form part of the molecule to be transported. In some embodiments, the NLS may be fused to the molecule by a covalent bond, hydrogen bonds or ionic interactions. In some embodiments, the NLS may be fused to the molecule via a linker.

“β2M” or “B2M,” as used herein, refers to nucleic acid sequence or protein sequence of “β-2 microglobulin;” the human gene has accession number NC_000015 (range 44711492 . . . 44718877), reference GRCh38.p13. The B2M protein is associated with MHC class I molecules as a heterodimer on the surface of nucleated cells and is required for MHC class I protein expression.

“CIITA” or “CIITA” or “C2TA,” as used herein, refers to the nucleic acid sequence or protein sequence of “class II major histocompatibility complex transactivator;” the human gene has accession number NC_000016.10 (range 10866208 . . . 10941562), reference GRCh38.p13. The CIITA protein in the nucleus acts as a positive regulator of MHC class II gene transcription and is required for MHC class II protein expression.

As used herein, “MHC” or “MHC molecule(s)” or “MHC protein” or “MHC complex(es),” refers to a major histocompatibility complex molecule (or plural), and includes, e.g., MHC class I and MHC class II molecules. In humans, MHC molecules are referred to as “human leukocyte antigen” complexes or “HLA molecules” or “HLA protein.” The use of terms “MHC” and “HLA” are not meant to be limiting; as used herein, the term “MHC” may be used to refer to human MHC molecules, i.e., HLA molecules. Therefore, the terms “MHC” and “HLA” are used interchangeably herein.

The term “HLA-A,” as used herein in the context of HLA-A protein, refers to the MHC class I protein molecule, which is a heterodimer consisting of a heavy chain (encoded by the HLA-A gene) and a light chain (i.e., beta-2 microglobulin). The term “HLA-A” or “HLA-A gene,” as used herein in the context of nucleic acids refers to the gene encoding the heavy chain of the HLA-A protein molecule. The HLA-A gene is also referred to as “HLA class I histocompatibility, A alpha chain;” the human gene has accession number NC_000006.12 (29942532 . . . 29945870). The HLA-A gene is known to have thousands of different versions (also referred to as “alleles”) across the population (and an individual may receive two different alleles of the HLA-A gene). A public database for HLA-A alleles, including sequence information, may be accessed at IPD-IMGT/HLA: https://www.ebi.ac.uk/ipd/imgt/hla/. All alleles of HLA-A are encompassed by the terms “HLA-A” and “HLA-A gene.”

“HLA-B” as used herein in the context of nucleic acids refers to the gene encoding the heavy chain of the HLA-B protein molecule. The HLA-B is also referred to as “HLA class I histocompatibility, B alpha chain;” the human gene has accession number NC_000006.12 (31353875 . . . 31357179).

“HLA-C” as used herein in the context of nucleic acids refers to the gene encoding the heavy chain of the HLA-C protein molecule. The HLA-C is also referred to as “HLA class I histocompatibility, C alpha chain;” the human gene has accession number NC_000006.12 (31268749 . . . 31272092).

“TRBC1” and “TRBC2” as used herein in the context of nucleic acids refer to two homologous genes encoding the T-cell receptor β-chain. “TRBC” or “TRBC1/2” is used herein to refer to TRBC1 and TRBC2. The human wild-type TRBC1 sequence is available at NCBI Gene ID: 28639; Ensembl: ENSG0000021 1751. T-cell receptor Beta Constant, V_segment Translation Product, BV05SIJ2.2, TCRBC1, and TCRB are gene synonyms for TRBC1. The human wild-type TRBC2 sequence is available at NCBI Gene ID: 28638; Ensembl: ENSG00000211772. T-cell receptor Beta Constant, V_segment Translation Product, and TCRBC2 are gene synonyms for TRBC2.

“TRAC” as used herein in the context of nucleic acids refers to the gene encoding the T-cell receptor α-chain. The human wild-type TRAC sequence is available at NCBI Gene ID: 28755; Ensembl: ENSG00000277734. T-cell receptor Alpha Constant, TCRA, IMD7, TRCA and TRA are gene synonyms for TRAC.

As used herein, the term “homozygous” refers to having two identical alleles of a particular gene.

As used herein, “treatment” refers to any administration or application of a therapeutic for disease or disorder in a subject, and includes inhibiting the disease, arresting its development, relieving one or more symptoms of the disease, curing the disease, or preventing one or more symptoms of the disease, including reoccurrence of the symptom.

As used herein, “delivering” and “administering” are used interchangeably, and include ex vivo and in vivo applications.

Co-administration, as used herein, means that a plurality of substances are administered sufficiently close together in time so that the agents act together. Co-administration encompasses administering substances together in a single formulation and administering substances in separate formulations close enough in time so that the agents act together.

As used herein, the phrase “pharmaceutically acceptable” means that which is useful in preparing a pharmaceutical composition that is generally non-toxic and is not biologically undesirable and that are not otherwise unacceptable for pharmaceutical use. Pharmaceutically acceptable generally refers to substances that are non-pyrogenic. Pharmaceutically acceptable can refer to substances that are sterile, especially for pharmaceutical substances that are for injection or infusion.

As used herein, a “subject” refers to any member of the animal kingdom. In some embodiments, “subject” refers to humans. In some embodiments, “subject” refers to non-human animals. In some embodiments, “subject” refers to primates. In some embodiments, subjects include, but are not limited to, mammals, birds, reptiles, amphibians, fish, insects, and/or worms. In certain embodiments, the non-human subject is a mammal (e.g., a rodent, a mouse, a rat, a rabbit, a monkey, a dog, a cat, a sheep, cattle, a primate, and/or a pig). In some embodiments, a subject may be a transgenic animal, genetically-engineered animal, and/or a clone. In certain embodiments of the present invention the subject is an adult, an adolescent or an infant. In some embodiments, terms “individual” or “patient” are used and are intended to be interchangeable with “subject”.

As used herein, “reduced or eliminated” expression of a protein on a cell refers to a partial or complete loss of expression of the protein relative to an unmodified cell. In some embodiments, the surface expression of a protein on a cell is measured by flow cytometry and has “reduced or eliminated” surface expression relative to an unmodified cell as evidenced by a reduction in fluorescence signal upon staining with the same antibody against the protein. A cell that has “reduced or eliminated” surface expression of a protein by flow cytometry relative to an unmodified cell may be referred to as “negative” for expression of that protein as evidenced by a fluorescence signal similar to a cell stained with an isotype control antibody. The “reduction or elimination” of protein expression can be measured by other known techniques in the field with appropriate controls known to those skilled in the art.

### II. Exemplary Compositions and Methods

In some embodiments, a nucleic acid is provided, the nucleic acid comprising an open reading frame encoding a polypeptide comprising a cytidine deaminase (e.g., A3A) and an RNA-guided nickase, wherein the polypeptide does not comprise a uracil glycosylase inhibitor (UGI). In some embodiments, the nucleic acid is DNA or RNA. In some embodiments, the nucleic acid is mRNA. In some embodiments, a polypeptide encoded by the mRNA is provided.

In some embodiments, a polypeptide or an mRNA encoding the polypeptide, are provided, the polypeptide comprising a cytidine deaminase and an RNA-guided nickase, wherein the polypeptide does not comprise a UGI. In some embodiments, the cytidine deaminase is A3A. In some embodiments, the RNA-guided nickase does not comprise a uracil glycosylase inhibitor (UGI). In some embodiments, a composition is provided comprising a first polypeptide, or an mRNA encoding a first polypeptide, comprising a cytidine deaminase (e.g., A3A) and an RNA-guided nickase; and a second polypeptide, or an mRNA encoding a second polypeptide, comprising a uracil glycosylase inhibitor (UGI), wherein the second polypeptide is different from the first polypeptide.

In some embodiments, a composition is provided comprising a first nucleic acid comprising a first open reading frame encoding a polypeptide comprising a cytidine deaminase (e.g., A3A) and an RNA-guided nickase, and a second nucleic acid comprising a second open reading frame encoding a uracil glycosylase inhibitor (UGI), wherein the second nucleic acid is different from the first nucleic acid. In some embodiments, the first nucleic acid encodes a polypeptide that does not comprise a UGI.

In some embodiments, methods of modifying a target gene are provided comprising administering the compositions described herein. In some embodiments, the method comprises delivering to a cell a first nucleic acid comprising a first open reading frame encoding a first polypeptide comprising a cytidine deaminase (e.g., A3A) and an RNA-guided nickase, and a second nucleic acid comprising a second open reading frame encoding a uracil glycosylase inhibitor (UGI), wherein the second nucleic acid is different from the first nucleic acid.

In some embodiments, the methods comprise delivering to a cell a polypeptide comprising a cytidine deaminase (e.g., A3A) and an RNA-guided nickase, or a nucleic acid encoding the polypeptide, and separately (e.g., not via the same nucleic acid construct) delivering to the cell a uracil glycosylase inhibitor (UGI), or a nucleic acid encoding the UGI.

In some embodiments, a molar ratio of the mRNA encoding UGI to the mRNA encoding the cytidine deaminase (e.g., A3A) and the RNA-guided nickase is from about 1:35 to from about 30:1. In some embodiments, the molar ratio is from about 1:25 to about 25:1. In some embodiments, the molar ratio is from about 1:20 to about 25:1. In some embodiments, the molar ratio is from about 1:10 to about 22:1. In some embodiments, the molar ratio is from about 1:5 to about 25:1. In some embodiments, the molar ratio is from about 1:1 to about 30:1. In some embodiments, the molar ratio is from about 2:1 to about 10:1. In some embodiments, the molar ratio is from about 5:1 to about 20:1. In some embodiments, the molar ratio is from about 1:1 to about 25:1. In some embodiments, the molar ratio may be about 1:35, 1:34, 1:33, 1:32, 1:31, 1:30, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, or 30:1. In some embodiments, the molar ratio is equal to or larger than about 1:1. In some embodiments the molar ratio is about 1:1. In some embodiments the molar ratio is about 2:1. In some embodiments the molar ratio is about 3:1. In some embodiments the molar ratio is about 4:1. In some embodiments the molar ratio is about 5:1. In some embodiments the molar ratio is about 6:1. In some embodiments the molar ratio is about 7:1. In some embodiments the molar ratio is about 8:1. In some embodiments the molar ratio is about 9:1. In some embodiments the molar ratio is about 10:1. In some embodiments the molar ratio is about 11:1. In some embodiments the molar ratio is about 12:1. In some embodiments the molar ratio is about 13:1. In some embodiments the molar ratio is about 14:1. In some embodiments the molar ratio is about 15:1. In some embodiments the molar ratio is about 16:1. In some embodiments the molar ratio is about 17:1. In some embodiments the molar ratio is about 18:1. In some embodiments the molar ratio is about 19:1. In some embodiments the molar ratio is about 20:1. In some embodiments the molar ratio is about 21:1. In some embodiments the molar ratio is about 22:1. In some embodiments the molar ratio is about 23:1. In some embodiments the molar ratio is about 24:1. In some embodiments the molar ratio is about 25:1.

Similarly, in some embodiments, the molar ratio discussed above for the mRNA encoding the UGI protein to the mRNA encoding the cytidine deaminase (e.g., A3A) and the RNA-guided nickase are similar if delivering protein.

For example, in some embodiments, a molar ratio of the UGI protein to be delivered to the cytidine deaminase (e.g., A3A) and the RNA-guided nickase to be delivered is from about 1:35 to from about 30:1. In some embodiments, the molar ratio is from about 1:1 to about 30:1.

In some embodiments, the molar ratio of the UGI peptide and the cytidine deaminase (e.g., A3A) and the RNA-guided nickase is from about 10:1 to about 50:1. In some embodiments, the molar ratio may be about 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, or 50:1. In some embodiments, the molar ratio is from about 10:1-about 40:1. In some embodiments the molar ratio is from about 10:1-about 30:1. In some embodiments the molar ratio is about 2:1. In some embodiments the molar ratio is from about 10:1-about 20:1. In some embodiments the molar ratio is from about 10:1-about 15:1. In some embodiments the molar ratio is about 15:1-about 50:1. In some embodiments the molar ratio is about 6:1. In some embodiments the molar ratio is about 20:1-about 50:1. In some embodiments the molar ratio is about 8:1. In some embodiments the molar ratio is about 30:1-about 50:1. In some embodiments the molar ratio is about 30:1-about 40:1. In some embodiments the molar ratio is about 11:1. In some embodiments the molar ratio is about 20:1-about 30:1.

In some embodiments, the composition described herein further comprises at least one gRNA. In some embodiments, a composition is provided that comprises an mRNA described herein and at least one gRNA. In some embodiments, the gRNA is a single guide RNA (sgRNA). In some embodiments, the gRNA is a dual guide RNA (dgRNA).

In some embodiments, the composition is capable of effecting genome editing upon administration to the subject.

A. UGI

Without being bound by any theory, providing a UGI together with a polypeptide comprising a deaminase may be helpful in the methods described herein by inhibiting cellular DNA repair machinery (e.g., UDG and downstream repair effectors) that recognize a uracil in DNA as a form of DNA damage or otherwise would excise or modify the uracil and/or surrounding nucleotides. It should be understood that the use of a UGI may increase the editing efficiency of an enzyme that is capable of deaminating C residues.

Suitable UGI protein and nucleotide sequences are provided herein and additional suitable UGI sequences are known to those in the art, and include, for example, those published in Wang et al., Uracil-DNA glycosylase inhibitor gene of bacteriophage PBS2 encodes a binding protein specific for uracil-DNA glycosylase. J. Biol. Chem. 264: 1163-1171(1989); Lundquist et al., Site-directed mutagenesis and characterization of uracil-DNA glycosylase inhibitor protein. Role of specific carboxylic amino acids in complex formation with Escherichia coli uracil-DNA glycosylase. J. Biol. Chem. 272:21408-21419(1997); Ravishankar et al., X-ray analysis of a complex of Escherichia coli uracil DNA glycosylase (EcUDG) with a proteinaceous inhibitor. The structure elucidation of a prokaryotic UDG. Nucleic Acids Res. 26:4880-4887(1998); and Putnam et al., Protein mimicry of DNA from crystal structures of the uracil-DNA glycosylase inhibitor protein and its complex with Escherichia coli uracil-DNA glycosylase. J. Mol. Biol. 287:331-346(1999), the entire contents of each are incorporated herein by reference. It should be appreciated that any proteins that are capable of inhibiting a uracil-DNA glycosylase base-excision repair enzyme are within the scope of the present disclosure. Additionally, any proteins that block or inhibit base-excision repair are also within the scope of this disclosure. In some embodiments, a uracil glycosylase inhibitor is a protein that binds uracil. In some embodiments, a uracil glycosylase inhibitor is a protein that binds uracil in DNA. In some embodiments, a uracil glycosylase inhibitor is a single-stranded binding protein. In some embodiments, a uracil glycosylase inhibitor is a catalytically inactive uracil DNA-glycosylase protein. In some embodiments, a uracil glycosylase inhibitor is a catalytically inactive uracil DNA-glycosylase protein that does not excise uracil from the DNA. In some embodiments, a uracil glycosylase inhibitor is a catalytically inactive UDG.

In some embodiments, a uracil glycosylase inhibitor (UGI) disclosed herein comprises an amino acid sequence with at least 80% to SEQ ID NO: 27 or 43. In some embodiments, any of the foregoing levels of identity is at least 90%, at least 95%, at least 98%, at least 99%, or 100%. In some embodiments, the UGI comprises an amino acid sequence with at least 90% identity to SEQ ID NO: 27 or 43. In some embodiments, the UGI comprises an amino acid sequence with at least 95% identity to SEQ ID NO: 27 or 43. In some embodiments, the UGI comprises an amino acid sequence with at least 98% identity to SEQ ID NO: 27 or 43. In some embodiments, the UGI comprises an amino acid sequence with at least 99% identity to SEQ ID NO: 27 or 43. In some embodiments, the UGI comprises the amino acid sequence of SEQ ID NO: 27 or 43.

B. Cytidine Deaminase

Cytidine deaminases encompass enzymes in the cytidine deaminase superfamily, and in particular, enzymes of the APOBEC family (APOBEC1, APOBEC2, APOBEC4, and APOBEC3 subgroups of enzymes), activation-induced cytidine deaminase (AID or AICDA) and CMP deaminases (see, e.g., Conticello et al., Mol. Biol. Evol. 22:367-77, 2005; Conticello, Genome Biol. 9:229, 2008; Muramatsu et al., J. Biol. Chem. 274: 18470-6, 1999); and Carrington et al., Cells 9:1690 (2020)).

In some embodiments, the cytidine deaminase disclosed herein is an enzyme of APOBEC family. In some embodiments, the cytidine deaminase disclosed herein is an enzyme of APOBEC1, APOBEC2, APOBEC4, and APOBEC3 subgroups. In some embodiments, the cytidine deaminase disclosed herein is an enzyme of APOBEC3 subgroup. In some embodiments, the cytidine deaminase disclosed herein is an APOBEC3A deaminase (A3A).

In some embodiments, the cytidine deaminase is:
(i) an enzyme of the APOBEC family, optionally an enzyme of APOBEC3 subgroup;
(ii) a cytidine deaminase comprising an amino acid sequence that is at least 80% identical to any one of SEQ ID NOs: 40, 41, and 960-1023;
(iii) a cytidine deaminase comprising an amino acid sequence that is at least 80% identical to any one of SEQ ID NOs: 40, 41, and 960-1013;
(iv) a cytidine deaminase comprising an amino acid sequence that is at least 80% identical to any one of SEQ ID NOs: 40, 41, 976, 977, 979, 980, 984-987, 993-1006, and 1009; or
(v) a cytidine deaminase comprising an amino acid sequence that is at least 80% identical to any one of SEQ ID NOs: 40, 976, 981, 984, 986, and 1014-1023.

In some embodiments, the cytidine deaminase is a cytidine deaminase comprising an amino acid sequence having at least 80%, 85% 87%, 90%, 95%, 98%, 99%, or 100% identity to SEQ ID NO: 40, 41, and 960-1023. In some embodiments, the cytidine deaminase is a cytidine deaminase comprising an amino acid sequence that is at least 80%, 85%, 87%, 90%, 95%, 98%, 99%, or 100% identical to any one of SEQ ID NOs: 40, 41, and 960-1013. In some embodiments, the cytidine deaminase is a cytidine deaminase comprising an amino acid sequence that is at least 80%, 85%, 87%, 90%, 95%, 98%, 99%, or 100% identical to any one of SEQ ID NOs: 40, 41, 976, 977, 979, 980, 984-987, 993-1006, and 1009. In some embodiments, the cytidine deaminase is a cytidine deaminase comprising an amino acid sequence that is at least 80%, 85%, 87%, 90%, 95%, 98%, 99%, or 100% identical to any one of SEQ ID NOs: 40, 976, 981, 984, 986, and 1014-1023. In some embodiments, the cytidine deaminase is a cytidine deaminase comprising an amino acid sequence that is at least 80%, 85%, 87%, 90%, 95%, 98%, 99%, or 100% identical to SEQ ID NO: 976, 977, 993-1006, and 1009.

1. APOBEC3A Deaminase

In some embodiments, an APOBEC3A deaminase (A3A) disclosed herein is a human A3A. In some embodiments, the A3A is a wild-type A3A.

In some embodiment, the A3A is an A3A variant. A3A variants share homology to wild-type A3A, or a fragment thereof. In some embodiments, a A3A variant has at least about 80% identity, at least about 85% identity, at least about 90% identity, at least about 95% identity, at least about 96% identity, at least about 97% identity, at least about 98% identity, at least about 99% identity, at least about 99.5% identity, or at least about 99.9% identity to a wild type A3A. In some embodiments, the A3A variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a wild type A3A. In some embodiments, the A3A variant comprises a fragment of an A3A, such that the fragment has at least about 80% identity, at least about 90% identity, at least about 95% identity, at least about 96% identity, at least about 97% identity, at least about 98% identity, at least about 99% identity, at least about 99.5% identity, or at least about 99.9% identity to the corresponding fragment of a wild-type A3A.

In some embodiments, an A3A variant is a protein having a sequence that differs from a wild-type A3A protein by one or several mutations, such as substitutions, deletions, insertions, one or several single point substitutions. In some embodiments, a shortened A3A sequence could be used, e.g., by deleting N-terminal, C-terminal, or internal amino acids. In some embodiments, a shortened A3A sequence is used where one to four amino acids at the C-terminus of the sequence is deleted. In some embodiments, an APOBEC3A (such as a human APOBEC3A) has a wild-type amino acid position 57 (as numbered in the wild-type sequence). In some embodiments, an APOBEC3A (such as a human APOBEC3A) has an asparagine at amino acid position 57 (as numbered in the wild-type sequence).

In some embodiments, the wild-type A3A is a human A3A (UniPROT accession ID: p319411, SEQ ID NO: 40).

In some embodiments, the A3A disclosed herein comprises an amino acid sequence having at least 80% identity to SEQ ID NO: 40. In some embodiments, the level of identity is at least 85%, at least 87%, at least 90%, at least 95%, at least 98%, at least 99%, or 100%. In some embodiments, the A3A comprises an amino acid sequence having at least 87% identity to SEQ ID NO: 40. In some embodiments, the A3A comprises an amino acid sequence with at least 90% identity to SEQ ID NO: 40. In some embodiments, the A3A comprises an amino acid sequence with at least 95% identity to SEQ ID NO: 40. In some embodiments, the A3A comprises an amino acid sequence with at least 98% identity to SEQ ID NO: 40. In some embodiments, the A3A comprises an amino acid sequence with at least 99% identity to A3A ID NO: 40. In some embodiments, the A3A comprises the amino acid sequence of SEQ ID NO: 40.

C. Linkers

In some embodiments, the polypeptide comprising the A3A and the RNA-guided nickase described herein further comprises a linker that connects the A3A and the RNA-guided nickase. In some embodiments, the linker is an organic molecule, polymer, or chemical moiety. In some embodiments, the linker is a peptide linker. In some embodiments, the nucleic acid encoding the polypeptide comprising the A3A and the RNA-guided nickase further comprises a sequence encoding the peptide linker. mRNAs encoding the A3A-linker-RNA-guided nickase fusion protein are provided.

In some embodiments, the peptide linker is any stretch of amino acids having at least 1, at least 2, at least 3, at least 4, at least 5, at least 6, at least 7, at least 8, at least 9, at least 10, at least 15, at least 20, at least 25, at least 30, at least 40, at least 50, or more amino acids.

In some embodiments, the peptide linker is the 16 residue “XTEN” linker, or a variant thereof (See, e.g., the Examples; and Schellenberger et al. A recombinant polypeptide extends the in vivo half-life of peptides and proteins in a tunable manner. Nat. Biotechnol. 27, 1186-1190 (2009)). In some embodiments, the XTEN linker comprises a sequence that is any one of SGSETPGTSESATPES (SEQ ID NO: 46), SGSETPGTSESA (SEQ ID NO: 47), or SGSETPGTSESATPEGGSGGS (SEQ ID NO: 48). In some embodiments, the XTEN linker consists of the sequence SGSETPGTSESATPES (SEQ ID NO: 46), SGSETPGTSESA (SEQ ID NO: 47), or SGSETPGTSESATPEGGSGGS (SEQ ID NO: 48).

In some embodiments, the peptide linker comprises a (GGGGS)ₙ (e.g., SEQ ID NOs: 212, 216, 221, 240), a (G)ₙ, an (EAAAK)ₙ (e.g., SEQ ID NOs: 213, 219, 267), a (GGS)ₙ, an SGSETPGTSESATPES (SEQ ID NO: 46) motif (see. e.g., Guilinger J P, Thompson D B, Liu D R. Fusion of catalytically inactive Cas9 to FokI nuclease improves the specificity of genome modification. Nat. Biotechnol. 2014; 32(6): 577-82; the entire contents are incorporated herein by reference), or an (XP)ₙ motif, or a combination of any of these, wherein n is independently an integer between 1 and 30. See, WO2015089406, e.g., paragraph [0012], the entire content of which is incorporated herein by reference.

In some embodiments, the peptide linker comprises one or more sequences selected from SEQ ID NOs: 46-59, 61 and 211-272. In some embodiments, the peptide linker comprises one or more sequences selected from SEQ ID NO: 46, SEQ ID NO: 47, SEQ ID NO: 48, SEQ ID NO: 268, SEQ ID NO: 269, SEQ ID NO: 270. SEQ ID NO: 271 and SEQ ID NO: 272. In some embodiments, the peptide linker comprises a sequence of SEQ ID NO: 268.

D. RNA-Guided Nickase

In some embodiments, an RNA-guided nickase disclosed herein is a Cas nickase. In some embodiments, a RNA-guided nickase is from a specific Cas nuclease with its catalytic domain(s) being inactivated. In some embodiments, the RNA-guided nickase is a Class 2 Cas nickase, such as a Cas9 nickase or a Cpf1 nickase. In some embodiments, the RNA-guided nickase is an S. pyogenes Cas9 nickase. In some embodiments, the RNA-guided nickase is Neisseria meningitidis Cas9 nickase.

In some embodiments, the RNA-guided nickase is a modified Class 2 Cas protein or derived from a Class 2 Cas protein. In some embodiments, the RNA-guided nickase is modified or derived from a Cas protein, such as a Class 2 Cas nuclease (which may be, e.g., a Cas nuclease of Type II, V, or VI). Class 2 Cas nuclease include, for example, Cas9, Cpf1, C2c1, C2c2, and C2c3 proteins and modifications thereof. Examples of Cas9 nucleases include those of the type II CRISPR systems of S. pyogenes. S. aureus, and other prokaryotes (see. e.g., the list in the next paragraph), and modified (e.g., engineered or mutant) versions thereof. See, e.g., US2016/0312198 A1; US 2016/0312199 A1, which is incorporated by reference in its entirety. Other examples of Cas nucleases include a Csm or Cmr complex of a type III CRISPR system or the Cas10, Csm1, or Cmr2 subunit thereof; and a Cascade complex of a type I CRISPR system, or the Cas3 subunit thereof. In some embodiments, the Cas nuclease may be from a Type-IIA, Type-IIB, or Type-IIC system. For discussion of various CRISPR systems and Cas nucleases, see, e.g., Makarova et al., NAT. REV. MICROBIOL. 9:467-477 (2011); Makarova et al., NAT. REV. MICROBIOL, 13: 722-36 (2015); Shmakov et al., MOLECULAR CELL, 60:385-397 (2015).

A Cas nickase described herein may be a nickase form of a Cas nuclease from the species including, but not limited to, Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gammaproteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acidaminococcus sp., Lachnospiraceae bacterium ND2006, or Acaryochloris marina.

In some embodiments, the Cas nickase is a nickase form of the Cas9 nuclease from Streptococcus pyogenes. In some embodiments, the Cas nickase is a nickase form of the Cas9 nuclease from Streptococcus thermophilus. In some embodiments, the Cas nickase is a nickase form of the Cas9 nuclease from Neisseria meningitidis. See e.g., WO/2020081568, describing an Nme2Cas9 D16A nickase. In some embodiments, the Cas nickase is a nickase form of the Cas9 nuclease from Staphylococcus aureus. In some embodiments, the Cas nickase is a nickase form of the Cpf1 nuclease from Francisella novicida. In some embodiments, the Cas nickase is a nickase form of the Cpf1 nuclease from Acidaminococcus sp. In some embodiments, the Cas nickase is a nickase form of the Cpf1 nuclease from Lachnospiraceae bacterium ND2006. In further embodiments, the Cas nickase is a nickase form of the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus Methanoplasma termitum, Eubacterium eligens, Moraxella bovoculi, Leptospira inadai, Porphyromonas crevioricanis, Prevotella disiens, or Porphyromonas macacae. In certain embodiments, the Cas nickase is a nickase form of a Cpf1 nuclease from an Acidaminococcus or Lachnospiraceae. As discussed elsewhere, a nickase may be derived from (i.e. related to) a specific Cas nuclease in that the nickase is a form of the nuclease in which one of its two catalytic domains is inactivated, e.g., by mutating an active site residue essential for nucleolysis, such as D10, H840, or N863 in Spy Cas9. One skilled in the art will be familiar with techniques for easily identifying corresponding residues in other Cas proteins, such as sequence alignment and structural alignment, which is discussed in detail below.

In other embodiments, the Cas nickase may relate to a Type-I CRISPR/Cas system. In some embodiments, the Cas nickase may be a component of the Cascade complex of a Type-I CRISPR/Cas system. In some embodiments, the Cas nickase may be a Cas3 protein. In some embodiments, the Cas nickase may be from a Type-III CRISPR/Cas system.

In some embodiments, a Cas nickase is a nickase form of a Cas nuclease or a modified Cas nuclease in which an endonucleolytic active site is inactivated, e.g., by one or more alterations (e.g., point mutations) in a catalytic domain. See, e.g., U.S. Pat. No. 8,889,356 for discussion of Cas nickases and exemplary catalytic domain alterations.

Wild type S. pyogenes Cas9 has two catalytic domains: RuvC and HNH. The RuvC domain cleaves the non-target DNA strand, and the HNH domain cleaves the target strand of DNA. In some embodiments, a Cas nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell October 22:163(3): 759-771. In some embodiments, the Cas nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al. (2015). Further exemplary amino acid substitutions include D917A, E1006A, and D1255A (based on the Francisella novicida U112 Cpf1 (FnCpf1) sequence (UniProtKB—AOQ7Q2 (CPF1_FRATN)).

In some embodiments, a Cas nickase such as a Cas9 nickase has an inactivated RuvC or HNH domain. In some embodiments, a nickase is used having a RuvC domain with reduced activity. In some embodiments, a nickase is used having an inactive RuvC domain. In some embodiments, a nickase is used having an HNH domain with reduced activity. In some embodiments, a nickase is used having an inactive HNH domain.

In some embodiments, a Cas9 nickase has an active HNH nuclease domain and is able to cleave the non-targeted strand of DNA, i.e., the strand bound by the gRNA and has an inactive RuvC nuclease domain and is not able to cleave the targeted strand of the DNA, i.e., the strand where base editing by deaminase is desired.

An exemplary Cas9 nickase amino acid sequence is provided as SEQ ID NO: 70. An exemplary Cas9 nickase mRNA ORF sequence, which includes start and stop codons, is provided as SEQ ID NO: 71. An exemplary Cas9 nickase mRNA coding sequence, suitable for inclusion in a fusion protein, is provided as SEQ ID NO: 72.

In some embodiments, the RNA-guided nickase is a Class 2 Cas nickase described herein. In some embodiments, the RNA-guided nickase is a Cas9 nickase described herein.

In some embodiments, the RNA-guided nickase is an S. pyogenes Cas9 nickase described herein.

In some embodiments, the RNA-guided nickase is a D10A SpyCas9 nickase described herein. In some embodiments, the RNA-guided nickase comprises an amino acid sequence having at least 80%, 90%, 95%, 98%, or 99% identity to any one of SEQ ID NO: 70, 73, or 76. In some embodiments, the RNA-guided nickase comprises the amino acid sequence of SEQ ID NO: 70.

In some embodiments, the mRNA ORF sequence comprises encoding the RNA-guided nickase, which includes start and stop codons, comprises a nucleotide sequence having at least 80%, 90%, 95%, 98%, 99% or 100% identity to the nucleotide sequence of any one of SEQ ID NOs: 71, 74, or 77. In some embodiments, the mRNA sequence encoding the RNA-guided nickase comprises a nucleotide sequence having at least 80%, 90%, 95%, 98%, 99% or 100% identity to the nucleotide sequence of any one of SEQ ID NOs: 72, 75, or 78. In some embodiments, the level of identity is at least 90%. In some embodiments, the level of identity is at least 95%. In some embodiments, the level of identity is at least 98%. In some embodiments, the level of identity is at least 99%. In some embodiments, the level of identity is at least 100%. In some embodiments, the sequence encoding the RNA-guided nickase comprises the nucleotide sequence of any one of SEQ ID NOs: 71, 72, 74, 75, 77, or 78.

In some embodiments, the RNA-guided nickase is Neisseria meningitidis (Nme) Cas9 nickase described herein.

In some embodiments, the RNA-guided nickase is a D16A NmeCas9 nickase described herein. In some embodiments, the D16A NmeCas9 nickase is a D16A Nme2Cas9 nickase. In some embodiments, the D16A Nme2Cas9 nickase comprises an amino acid sequence at least 80%, 90%, 95%, 98%, 99% or 100% identical to SEQ ID NO: 387. In some embodiments, the sequence encoding the D16A Nme2Cas9 comprises a nucleotide sequence at least 80%, 90%, 95%, 98%, 99% or 100% identical to any one of SEQ ID NOs: 388-393.

E. Compositions Comprising a Cytidine Deaminase and an RNA-Guided Nickase

In some embodiments, an mRNA encoding a polypeptide comprising a cytidine deaminase and an RNA-guided nickase is provided, wherein the polypeptide does not comprise a uracil glycosylase inhibitor (UGI).

1. Exemplary Compositions

As described herein, compositions, methods, and uses are provided comprising an mRNA comprising an open reading frame encoding a polypeptide comprising a cytidine deaminase and an RNA-guided nickase, wherein the polypeptide does not comprise a uracil glycosylase inhibitor (UGI). For each exemplary composition described below, the mRNA does not comprise a UGI.

In some embodiments, an mRNA encoding a polypeptide comprising a cytidine deaminase and an RNA-guided nickase is provided. In some embodiments, an enzyme of APOBEC family and an RNA-guided nickase is provided. In some embodiments, the polypeptide comprises an enzyme of APOBEC1 subgroup and an RNA-guided nickase. In some embodiments, the polypeptide comprises an enzyme of APOBEC2 subgroup and an RNA-guided nickase. In some embodiments, the polypeptide comprises an enzyme of APOBEC4 subgroup and an RNA-guided nickase. In some embodiments, the polypeptide comprises an enzyme of APOBEC3 subgroup and an RNA-guided nickase.

In some embodiments, an mRNA encoding a polypeptide comprising a cytidine deaminase and an RNA-guided nickase is provided. In some embodiments, an enzyme of APOBEC family and a D10A SpyCas9 nickase is provided. In some embodiments, the polypeptide comprises an enzyme of APOBEC1 subgroup and a D10A SpyCas9 nickase. In some embodiments, the polypeptide comprises an enzyme of APOBEC2 subgroup and a D10A SpyCas9 nickase. In some embodiments, the polypeptide comprises an enzyme of APOBEC4 subgroup and a D10A SpyCas9 nickase. In some embodiments, the polypeptide comprises an enzyme of APOBEC3 subgroup and a D10A SpyCas9 nickase.

In some embodiments, an mRNA encoding a polypeptide comprising a cytidine deaminase and an RNA-guided nickase is provided. In some embodiments, an enzyme of APOBEC family and a D16A NmeCas9 nickase is provided. In some embodiments, an enzyme of APOBEC family and a D16A Nme2Cas9 nickase is provided. In some embodiments, the polypeptide comprises an enzyme of APOBEC1 subgroup and a D16A Nme2Cas9 nickase. In some embodiments, the polypeptide comprises an enzyme of APOBEC2 subgroup and a D16A Nme2Cas9 nickase. In some embodiments, the polypeptide comprises an enzyme of APOBEC4 subgroup and a D16A Nme2Cas9 nickase. In some embodiments, the polypeptide comprises an enzyme of APOBEC3 subgroup and a D16A Nme2Cas9 nickase.

In some embodiments, the polypeptide lacks a UGI.

In some embodiments, the cytidine deaminase and the RNA-guided nickase are linked via a linker. In some embodiments, the cytidine deaminase and the RNA-guided nickase are linked via a peptide linker. In some embodiments, the peptide linker comprises one or more sequences selected from SEQ ID NOs: 46-59, 61 and 211-272.

In some embodiments, the polypeptide further comprises one or more additional heterologous functional domains. In some embodiments, the polypeptide further comprises one or more nuclear localization sequences (NLSs) (described herein) at the C-terminal of the polypeptide or the N-terminal of the polypeptide.

In some embodiments, an mRNA encoding a polypeptide comprising a cytidine deaminase and an RNA-guided nickase is provided. In some embodiments, an enzyme of APOBEC family and an RNA-guided nickase is provided. In some embodiments, the polypeptide comprises an enzyme of APOBEC1 subgroup and an RNA-guided nickase. In some embodiments, the polypeptide comprises an enzyme of APOBEC2 subgroup and an RNA-guided nickase. In some embodiments, the polypeptide comprises an enzyme of APOBEC4 subgroup and an RNA-guided nickase. In some embodiments, the polypeptide comprises an enzyme of APOBEC3 subgroup and an RNA-guided nickase.

In some embodiments, an mRNA encoding a polypeptide comprising a cytidine deaminase and an RNA-guided nickase is provided. In some embodiments, an enzyme of APOBEC family and a D10A SpyCas9 nickase, wherein the enzyme of APOBEC family and the D10A SpyCas9 nickase are fused via a linker. In some embodiments, the polypeptide comprises an enzyme of APOBEC family and a D10A SpyCas9 nickase, and a nuclear localization sequence (NLS) at the C-terminus of the fused polypeptide. In some embodiments, the polypeptide comprises an enzyme of APOBEC family and a D10A SpyCas9 nickase, and a NLS at the N-terminus of the fused polypeptide. In some embodiments, the polypeptide comprises an enzyme of APOBEC family and a D10A SpyCas9 nickase, wherein the enzyme of APOBEC family and the D10A SpyCas9 nickase are fused via a linker, and a NLS fused to the C-terminus of the D10A SpyCas9 nickase, optionally via a linker. In some embodiments, the polypeptide comprises an enzyme of APOBEC family and a D10A SpyCas9 nickase, wherein the enzyme of APOBEC family and the D10A SpyCas9 nickase are fused via a linker, and a NLS fused to the C-terminus of the D10A SpyCas9 nickase, optionally via a linker.

In some embodiments, the polypeptide comprises an enzyme of APOBEC family and a D16A NmeCas9 nickase, wherein the enzyme of APOBEC family and the D16A NmeCas9 nickase are fused via a linker. In some embodiments, the polypeptide comprises an enzyme of APOBEC family and a D16A Nme2Cas9 nickase, wherein the enzyme of APOBEC family and the D16A Nme2Cas9 nickase are fused via a linker. In some embodiments, the polypeptide comprises an enzyme of APOBEC family and a D16A Nme2Cas9 nickase, and a nuclear localization sequence (NLS) at the C-terminus of the fused polypeptide. In some embodiments, the polypeptide comprises an enzyme of APOBEC family and a D16A Nme2Cas9 nickase, and a NLS at the N-terminus of the fused polypeptide. In some embodiments, the polypeptide comprises an enzyme of APOBEC family and a D16A Nme2Cas9 nickase, wherein the enzyme of APOBEC family and the D16A Nme2Cas9 nickase are fused via a linker, and a NLS fused to the C-terminus of the D16A Nme2Cas9 nickase, optionally via a linker. In some embodiments, the polypeptide comprises an enzyme of APOBEC family and a D16A Nme2Cas9 nickase, wherein the enzyme of APOBEC family and the D16A Nme2Cas9 nickase are fused via a linker, and a NLS fused to the C-terminus of the D16A Nme2Cas9 nickase, optionally via a linker.

In some embodiments, the polypeptide comprises an enzyme of APOBEC1 subgroup and a D10A SpyCas9 nickase, wherein the enzyme of APOBEC1 subgroup and the D10A SpyCas9 nickase are fused via a linker. In some embodiments, the polypeptide comprises an enzyme of APOBEC1 subgroup and a D10A SpyCas9 nickase, and a nuclear localization sequence (NLS) at the C-terminus of the fused polypeptide. In some embodiments, the polypeptide comprises an enzyme of APOBEC1 subgroup and a D10A SpyCas9 nickase, and a NLS at the N-terminus of the fused polypeptide. In some embodiments, the polypeptide comprises an enzyme of APOBEC1 subgroup and a D10A SpyCas9 nickase, wherein the enzyme of APOBEC1 subgroup and the D10A SpyCas9 nickase are fused via a linker, and a NLS fused to the C-terminus of the D10A SpyCas9 nickase, optionally via a linker. In some embodiments, the polypeptide comprises an enzyme of APOBEC1 subgroup and a D10A SpyCas9 nickase, wherein the enzyme of APOBEC1 subgroup and the D10A SpyCas9 nickase are fused via a linker, and a NLS fused to the C-terminus of the D10A SpyCas9 nickase, optionally via a linker.

In some embodiments, the polypeptide comprises an enzyme of APOBEC1 subgroup and a D16A Nme2Cas9 nickase, wherein the enzyme of APOBEC1 subgroup and the D16A Nme2Cas9 nickase are fused via a linker. In some embodiments, the polypeptide comprises an enzyme of APOBEC1 subgroup and a D16A Nme2Cas9 nickase, wherein the enzyme of APOBEC1 subgroup and the D16A Nme2Cas9 nickase are fused via a linker. In some embodiments, the polypeptide comprises an enzyme of APOBEC1 subgroup and a D16A Nme2Cas9 nickase, and a nuclear localization sequence (NLS) at the C-terminus of the fused polypeptide. In some embodiments, the polypeptide comprises an enzyme of APOBEC1 subgroup and a D16A Nme2Cas9 nickase, and a NLS at the N-terminus of the fused polypeptide. In some embodiments, the polypeptide comprises an enzyme of APOBEC1 subgroup and a D16A Nme2Cas9 nickase, wherein the enzyme of APOBEC1 subgroup and the D16A Nme2Cas9 nickase are fused via a linker, and a NLS fused to the C-terminus of the D16A Nme2Cas9 nickase, optionally via a linker. In some embodiments, the polypeptide comprises an enzyme of APOBEC1 subgroup and a D16A Nme2Cas9 nickase, wherein the enzyme of APOBEC1 subgroup and the D16A Nme2Cas9 nickase are fused via a linker, and a NLS fused to the C-terminus of the D16A Nme2Cas9 nickase, optionally via a linker.

In some embodiments, the polypeptide comprises an enzyme of APOBEC3 subgroup and a D10A SpyCas9 nickase, wherein the enzyme of APOBEC3 subgroup and the D10A SpyCas9 nickase are fused via a linker. In some embodiments, the polypeptide comprises an enzyme of APOBEC3 subgroup and a D10A SpyCas9 nickase, and a nuclear localization sequence (NLS) at the C-terminus of the fused polypeptide. In some embodiments, the polypeptide comprises an enzyme of APOBEC3 subgroup and a D10A SpyCas9 nickase, and a NLS at the N-terminus of the fused polypeptide. In some embodiments, the polypeptide comprises an enzyme of APOBEC3 subgroup and a D10A SpyCas9 nickase, wherein the enzyme of APOBEC3 subgroup and the D10A SpyCas9 nickase are fused via a linker, and a NLS fused to the C-terminus of the D10A SpyCas9 nickase, optionally via a linker. In some embodiments, the polypeptide comprises an enzyme of APOBEC3 subgroup and a D10A SpyCas9 nickase, wherein the enzyme of APOBEC3 subgroup and the D10A SpyCas9 nickase are fused via a linker, and a NLS fused to the C-terminus of the D10A SpyCas9 nickase, optionally via a linker.

In some embodiments, the polypeptide comprises an enzyme of APOBEC3 subgroup and a D16A Nme2Cas9 nickase, wherein the enzyme of APOBEC3 subgroup and the D16A Nme2Cas9 nickase are fused via a linker. In some embodiments, the polypeptide comprises an enzyme of APOBEC3 subgroup and a D16A Nme2Cas9 nickase, wherein the enzyme of APOBEC3 subgroup and the D16A Nme2Cas9 nickase are fused via a linker. In some embodiments, the polypeptide comprises an enzyme of APOBEC3 subgroup and a D16A Nme2Cas9 nickase, and a nuclear localization sequence (NLS) at the C-terminus of the fused polypeptide. In some embodiments, the polypeptide comprises an enzyme of APOBEC3 subgroup and a D16A Nme2Cas9 nickase, and a NLS at the N-terminus of the fused polypeptide. In some embodiments, the polypeptide comprises an enzyme of APOBEC3 subgroup and a D16A Nme2Cas9 nickase, wherein the enzyme of APOBEC3 subgroup and the D16A Nme2Cas9 nickase are fused via a linker, and a NLS fused to the C-terminus of the D16A Nme2Cas9 nickase, optionally via a linker. In some embodiments, the polypeptide comprises an enzyme of APOBEC3 subgroup and a D16A Nme2Cas9 nickase, wherein the enzyme of APOBEC3 subgroup and the D16A Nme2Cas9 nickase are fused via a linker, and a NLS fused to the C-terminus of the D16A Nme2Cas9 nickase, optionally via a linker.

In some embodiments, the polypeptide comprises a D10A SpyCas9 nickase, a linker comprising the amino acid sequence of SEQ ID NO: 268, and a cytidine deaminase comprising an amino acid sequence that is at least 85% identical to any one of SEQ ID NOs: 40, 41, 976, 977, 979, 980, 984-987, 993-1006, and 1009. In some embodiments, the polypeptide comprises a D10A SpyCas9 nickase, a linker comprising the amino acid sequence of SEQ ID NO: 269, and a cytidine deaminase comprising an amino acid sequence that is at least 85% identical to any one of SEQ ID NOs: 40, 41, 976, 977, 979, 980, 984-987, 993-1006, and 1009. In some embodiments, the polypeptide comprises a D10A SpyCas9 nickase, a linker comprising the amino acid sequence of SEQ ID NO: 270, and a cytidine deaminase comprising an amino acid sequence that is at least 85% identical to any one of SEQ ID NOs: 40, 41, 976, 977, 979, 980, 984-987, 993-1006, and 1009. In some embodiments, the polypeptide comprises a D10A SpyCas9 nickase, a linker comprising the amino acid sequence of SEQ ID NO: 271, and a cytidine deaminase comprising an amino acid sequence that is at least 85% identical to any one of SEQ ID NOs: 40, 41, 976, 977, 979, 980, 984-987, 993-1006, and 1009. In some embodiments, the polypeptide comprises a D10A SpyCas9 nickase, a linker comprising the amino acid sequence of SEQ ID NO: 272, and a cytidine deaminase comprising an amino acid sequence that is at least 85% identical to any one of SEQ ID NOs: 40, 41, 976, 977, 979, 980, 984-987, 993-1006, and 1009. In any of the foregoing embodiments, the D10A SpyCas9 nickase may comprise an amino acid sequence that is at least 85%, at least 90%, at least 95%, at least 98%, at least 99%, or 100% identical to any one of SEQ ID NOs: 70, 73, or 76.

In some embodiments, the polypeptide comprises a D16A Nme2Cas9 nickase, a linker comprising the amino acid sequence of SEQ ID NO: 268, and a cytidine deaminase comprising an amino acid sequence that is at least 85% identical to any one of SEQ ID NOs: 40, 41, 976, 977, 979, 980, 984-987, 993-1006, and 1009. In some embodiments, the polypeptide comprises a D16A Nme2Cas9 nickase, a linker comprising the amino acid sequence of SEQ ID NO: 269, and a cytidine deaminase comprising an amino acid sequence that is at least 85% identical to any one of SEQ ID NOs: 40, 41, 976, 977, 979, 980, 984-987, 993-1006, and 1009. In some embodiments, the polypeptide comprises a D16A Nme2Cas9 nickase, a linker comprising the amino acid sequence of SEQ ID NO: 270, and a cytidine deaminase comprising an amino acid sequence that is at least 85% identical to any one of SEQ ID NOs: 40, 41, 976, 977, 979, 980, 984-987, 993-1006, and 1009. In some embodiments, the polypeptide comprises a D16A Nme2Cas9 nickase, a linker comprising the amino acid sequence of SEQ ID NO: 271, and a cytidine deaminase comprising an amino acid sequence that is at least 85% identical to any one of SEQ ID NOs: 40, 41, 976, 977, 979, 980, 984-987, 993-1006, and 1009. In some embodiments, the polypeptide comprises a D16A Nme2Cas9 nickase, a linker comprising the amino acid sequence of SEQ ID NO: 272, and a cytidine deaminase comprising an amino acid sequence that is at least 85% identical to any one of SEQ ID NOs: 40, 41, 976, 977, 979, 980, 984-987, 993-1006, and 1009. In any of the foregoing embodiments, the D16A Nme2Cas9 nickase may comprise an amino acid sequence that is at least 85%, at least 90%, at least 95%, at least 98%, at least 99%, or 100% identical to SEQ ID NO: 387.

In some embodiments, the polypeptide comprises a D10A SpyCas9 nickase, a linker comprising the amino acid sequence of SEQ ID NO: 268, and a cytidine deaminase comprising an amino acid sequence selected from any one of SEQ ID NOs: 40, 41, 976, 977, 979, 980, 984-987, 993-1006, and 1009. In some embodiments, the polypeptide comprises a D10A SpyCas9 nickase, a linker comprising the amino acid sequence of SEQ ID NO: 269, and a cytidine deaminase comprising an amino acid sequence selected from any one of SEQ ID NOs: 40, 41, 976, 977, 979, 980, 984-987, 993-1006, and 1009. In some embodiments, the polypeptide comprises a D10A SpyCas9 nickase, a linker comprising the amino acid sequence of SEQ ID NO: 270, and a cytidine deaminase comprising an amino acid sequence selected from any one of SEQ ID NOs: 40, 41, 976, 977, 979, 980, 984-987, 993-1006, and 1009. In some embodiments, the polypeptide comprises a D10A SpyCas9 nickase, a linker comprising the amino acid sequence of SEQ ID NO: 271, and a cytidine deaminase comprising an amino acid sequence selected from any one of SEQ ID NOs: 40, 41, 976, 977, 979, 980, 984-987, 993-1006, and 1009. In some embodiments, the polypeptide comprises a D10A SpyCas9 nickase, a linker comprising the amino acid sequence of SEQ ID NO: 272, and a cytidine deaminase comprising an amino acid sequence selected from any one of SEQ ID NOs: 40, 41, 976, 977, 979, 980, 984-987, 993-1006, and 1009. In any of the foregoing embodiments, the D10A SpyCas9 comprises an amino acid sequence that is at least 85%, at least 90%, at least 95%, at least 98%, at least 99%, or 100% identical to any one of SEQ ID NOs: 70, 73, or 76.

In some embodiments, the polypeptide comprises a D16A Nme2Cas9 nickase, a linker comprising the amino acid sequence of SEQ ID NO: 268, and a cytidine deaminase comprising an amino acid sequence selected from any one of SEQ ID NOs: 40, 41, 976, 977, 979, 980, 984-987, 993-1006, and 1009. In some embodiments, the polypeptide comprises a D16A Nme2Cas9 nickase, a linker comprising the amino acid sequence of SEQ ID NO: 269, and a cytidine deaminase comprising an amino acid sequence selected from any one of SEQ ID NOs: 40, 41, 976, 977, 979, 980, 984-987, 993-1006, and 1009. In some embodiments, the polypeptide comprises a D16A Nme2Cas9 nickase, a linker comprising the amino acid sequence of SEQ ID NO: 270, and a cytidine deaminase comprising an amino acid sequence selected from any one of SEQ ID NOs: 40, 41, 976, 977, 979, 980, 984-987, 993-1006, and 1009. In some embodiments, the polypeptide comprises a D16A Nme2Cas9 nickase, a linker comprising the amino acid sequence of SEQ ID NO: 271, and a cytidine deaminase comprising an amino acid sequence selected from any one of SEQ ID NOs: 40, 41, 976, 977, 979, 980, 984-987, 993-1006, and 1009. In some embodiments, the polypeptide comprises a D16A Nme2Cas9 nickase, a linker comprising the amino acid sequence of SEQ ID NO: 272, and a cytidine deaminase comprising an amino acid sequence selected from any one of SEQ ID NOs: 40, 41, 976, 977, 979, 980, 984-987, 993-1006, and 1009. In any of the foregoing embodiments, the D16A Nme2Cas9 nickase comprises an amino acid sequence that is at least 85%, at least 90%6, at least 95%, at least 98%, at least 99% or 100% identical to SEQ ID NO: 387.

The polypeptide may be organized in any number of ways to form a single chain. The NLS can be N- or C-terminal, or both N- and C-terminals, and the cytidine deaminase can be N- or C-terminal as compared the RNA-guided nickase. In some embodiments, the polypeptide comprises, from N to C terminus, a cytidine deaminase, an optional linker, an RNA-guided nickase, and an optional NLS. In some embodiments, the polypeptide comprises, from N to C terminus, an RNA-guided nickase, an optional linker, a cytidine deaminase, and an optional NLS. In some embodiments, the polypeptide comprises, from N to C terminus, an optional NLS, an RNA-guided nickase, an optional linker, and a cytidine deaminase. In some embodiments, the polypeptide comprises, from N to C terminus, an optional NLS, an RNA-guided nickase, an optional linker, and a cytidine deaminase, and an optional NLS.

In some embodiments, the polypeptide comprises, from N to C terminus, an optional NLS, an enzyme of APOBEC family, an optional linker, an RNA-guided nickase, and an optional NLS. In some embodiments, the polypeptide comprises, from N to C terminus, an optional NLS, an RNA-guided nickase, an optional linker, an enzyme of APOBEC family and an optional NLS. In some embodiments, the polypeptide comprises, from N to C terminus, an optional NLS, an RNA-guided nickase, an optional linker, an enzyme of APOBEC family, and an optional NLS. In some embodiments, the polypeptide comprises, from N to C terminus, an optional NLS, an RNA-guided nickase, an optional linker, an enzyme of APOBEC family, and an optional NLS.

In some embodiments, the polypeptide comprises, from N to C terminus, an optional NLS, an enzyme of APOBEC3 subgroup, an optional linker, an RNA-guided nickase, and an optional NLS. In some embodiments, the polypeptide comprises, from N to C terminus, an optional NLS, an RNA-guided nickase, an optional linker, an enzyme of APOBEC3 subgroup and an optional NLS. In some embodiments, the polypeptide comprises, from N to C terminus, an optional NLS, an RNA-guided nickase, an optional linker, an enzyme of APOBEC3 subgroup, and an optional NLS. In some embodiments, the polypeptide comprises, from N to C terminus, an optional NLS, an RNA-guided nickase, an optional linker, an enzyme of APOBEC3 subgroup, and an optional NLS.

In some embodiments, the polypeptide comprises, from N to C terminus, an optional NLS, an enzyme of APOBEC family, an optional linker, a D10A SpyCas9 nickase or a D16A Nme2Cas9 nickase, and an optional NLS. In some embodiments, the polypeptide comprises, from N to C terminus, an optional NLS, a D10A SpyCas9 nickase or a D16A Nme2Cas9 nickase, an optional linker, an enzyme of APOBEC family and an optional NLS. In some embodiments, the polypeptide comprises, from N to C terminus, an optional NLS, a D10A SpyCas9 nickase or a D16A Nme2Cas9 nickase, an optional linker, an enzyme of APOBEC family, and an optional NLS. In some embodiments, the polypeptide comprises, from N to C terminus, an optional NLS, a D10A SpyCas9 nickase or a D16A Nme2Cas9 nickase, an optional linker, and an enzyme of APOBEC family, and an optional NLS.

In some embodiments, the polypeptide comprises, from N to C terminus, an optional NLS, an enzyme of APOBEC3 subgroup, an optional linker, a D10A SpyCas9 nickase or a D16A Nme2Cas9 nickase, and an optional NLS. In some embodiments, the polypeptide comprises, from N to C terminus, an optional NLS, a D10A SpyCas9 nickase or a D16A Nme2Cas9 nickase, an optional linker, an enzyme of APOBEC3 subgroup and an optional NLS. In some embodiments, the polypeptide comprises, from N to C terminus, an optional NLS, a D10A SpyCas9 nickase or a D16A Nme2Cas9 nickase, an optional linker, an enzyme of APOBEC3 subgroup, and an optional NLS. In some embodiments, the polypeptide comprises, from N to C terminus, an optional NLS, a D10A SpyCas9 nickase or a D16A Nme2Cas9 nickase, an optional linker, and an enzyme of APOBEC3 subgroup, and an optional NLS.

In some embodiments, the polypeptide comprises, from N to C terminus, an optional NLS, an enzyme of APOBEC3 subgroup, an optional linker, a D16A Nme2Cas9 nickase.

In some embodiments, the polypeptide comprises, from N to C terminus, (i) an optional NLS; (ii) a cytidine deaminase comprising an amino acid sequence that is at least 80% identical to any one of SEQ ID NOs: 40, 41, and 960-1023; (iii) a linker comprising one or more sequences selected from SEQ ID NOs: 46-59, 61 and 211-272, (iv) a D10A SpyCas9 nickase or a D16A Nme2Cas9 nickase, and (v) an optional NLS.

In some embodiments, the polypeptide comprises, from N to C terminus, (i) an optional NLS, (ii) a D10A SpyCas9 nickase or a D16A Nme2Cas9 nickase, (iii) a linker comprising one or more sequences selected from SEQ ID NOs: 46-59, 61 and 211-272, (iv) a cytidine deaminase comprising an amino acid sequence that is at least 80% identical to any one of SEQ ID NOs: 40, 41, and 960-1023, and (v) an optional NLS.

In some embodiments, the polypeptide comprises, from N to C terminus, (i) an optional NLS, (ii) a D10A SpyCas9 nickase or a D16A Nme2Cas9 nickase, (iii) a linker comprising one or more sequences selected from SEQ ID NOs: 46-59, 61 and 211-272, (iv) a cytidine deaminase comprising an amino acid sequence that is at least 80% identical to any one of SEQ ID NOs: 40, 41, and 960-1023, and (v) an optional NLS.

In some embodiments, the polypeptide comprises, from N to C terminus, (i) an optional NLS, (ii) a D10A SpyCas9 nickase or a D16A Nme2Cas9 nickase, (iii) a linker comprising one or more sequences selected from SEQ ID NOs: 46-59, 61 and 211-272, (iv) cytidine deaminase comprising an amino acid sequence that is at least 80% identical to any one of SEQ ID NOs: 40, 41, and 960-1023, and (v) an optional NLS.

In some embodiments, the polypeptide comprises, from N to C terminus, (i) an optional NLS, (ii) a cytidine deaminase comprising an amino acid sequence that is at least 80% identical to any one of SEQ ID NOs: 40, 41, and 960-1023; (iii) a linker comprising one or more sequences selected from SEQ ID NOs: 46-59, 61 and 211-272, (iv) a D10A SpyCas9 nickase or a D16A Nme2Cas9 nickase, and (v) an optional NLS.

In some embodiments, the polypeptide comprises, from N to C terminus, (i) an optional NLS, (ii) a D10A SpyCas9 nickase or a D16A Nme2Cas9 nickase, (iii) a linker comprising one or more sequences selected from SEQ ID NOs: 46-59, 61 and 211-272, (iv) a cytidine deaminase comprising an amino acid sequence that is at least 80% identical to any one of SEQ ID NOs: 40, 41, and 960-1023, and (v) an optional NLS.

In some embodiments, the polypeptide comprises, from N to C terminus, (i) an optional NLS, (ii) a D10A SpyCas9 nickase or a D16A Nme2Cas9 nickase, (iii) a linker comprising one or more sequences selected from SEQ ID NOs: 46-59, 61 and 211-272, (iv) a cytidine deaminase comprising an amino acid sequence that is at least 80% identical to any one of SEQ ID NOs: 40, 41, and 960-1023, and (v) an optional NLS.

In some embodiments, the polypeptide comprises, from N to C terminus, (i) an optional NLS, (ii) a D10A SpyCas9 nickase or a D16A Nme2Cas9 nickase, (iii) a linker comprising one or more sequences selected from SEQ ID NOs: 46-59, 61 and 211-272, and (iv) cytidine deaminase comprising an amino acid sequence that is at least 80% identical to any one of SEQ ID NOs: 40, 41, and 960-1023, and (v) an optional NLS.

2. Compositions Comprising an APOBEC3A Deaminase and an RNA-Guided Nickase

In some embodiments, an mRNA encoding a polypeptide comprising an APOBEC3A deaminase (A3A) and an RNA-guided nickase is provided. In some embodiments, the polypeptide comprises a human A3A and an RNA-guided nickase. In some embodiments, the polypeptide comprises a wild-type A3A and an RNA-guided nickase. In some embodiments, the polypeptide comprises an A3A variant and an RNA-guided nickase. In some embodiments, the polypeptide comprises an A3A and a Cas9 nickase. In some embodiments, the polypeptide comprises an A3A and a D10A SpyCas9 nickase. In some embodiments, the polypeptide comprises a human A3A and a D10A SpyCas9 nickase. In some embodiments, the polypeptide comprises an A3A variant and a D10A SpyCas9 nickase. In some embodiments, the polypeptide lacks a UGI. In some embodiments, the A3A and the RNA-guided nickase are linked via a linker. In some embodiments, the polypeptide further comprises one or more additional heterologous functional domains. In some embodiments, the polypeptide further comprises a nuclear localization sequence (NLS) (described herein) at the C-terminal of the polypeptide or the N-terminal of the polypeptide.

In some embodiments, the polypeptide comprises a human A3A and a D10A SpyCas9 nickase, wherein the human A3A and the D10A SpyCas9 nickase are fused via a linker. In some embodiments, the polypeptide comprises a human A3A and a D10A SpyCas9 nickase, and a nuclear localization sequence (NLS) at the C-terminus of the fused polypeptide. In some embodiments, the polypeptide comprises a human A3A and a D10A SpyCas9 nickase, and a NLS at the N-terminus of the fused polypeptide. In some embodiments, the polypeptide comprises a human A3A and a D10A SpyCas9 nickase, wherein the human A3A and the DOA SpyCas9 nickase are fused via a linker, and a NLS fused to the C-terminus of the DOA SpyCas9 nickase, optionally via a linker. In some embodiments, the polypeptide comprises a human A3A and a D10A SpyCas9 nickase, wherein the human A3A and the DOA SpyCas9 nickase are fused via a linker, and a NLS fused to the C-terminus of the D10A SpyCas9 nickase, optionally via a linker.

The polypeptide may be organized in any number of ways to form a single chain. The NLS can be N- or C-terminal, or both N- and C-terminals. and the A3A can be N- or C-terminal as compared the RNA-guided nickase. In some embodiments, the polypeptide comprises, from N to C terminus, an A3A, an optional linker, an RNA-guided nickase, and an optional NLS. In some embodiments, the polypeptide comprises, from N to C terminus, an RNA-guided nickase, an optional linker, an A3A, and an optional NLS. In some embodiments, the polypeptide comprises, from N to C terminus, an optional NLS, an RNA-guided nickase, an optional linker, and an A3A. In some embodiments, the polypeptide comprises, from N to C terminus, an optional NLS, an RNA-guided nickase, an optional linker, and an A3A, and an optional NLS.

In any of the foregoing embodiments, the polypeptide may comprise an amino acid sequence having at least 80% identity to SEQ ID NOs: 3 or 6. In some embodiments, any of the foregoing levels of identity is at least 85%, at least 90%, at least 95%, at least 98%, at least 99%, or 100%. In some embodiments, the polypeptide disclosed herein may comprise an amino acid sequence with at least 90% identity to SEQ ID NOs: 3 or 6. In some embodiments, the polypeptide disclosed herein may comprise an amino acid sequence with at least 95% identity to SEQ ID NOs: 3 or 6. In some embodiments, the polypeptide disclosed herein may comprise an amino acid sequence with at least 98% identity to SEQ ID NOs: 3 or 6. In some embodiments, the polypeptide disclosed herein may comprise an amino acid sequence with at least 99% identity to SEQ ID NOs: 3 or 6. In some embodiments, the polypeptide disclosed herein may comprise an amino acid sequence of SEQ ID NOs: 3 or 6.

In any of the foregoing embodiments, a nucleic acid sequence comprising an open reading frame encoding the polypeptide disclosed herein may comprise a nucleic acid sequence having at least 80% identity to SEQ ID NOs: 2 or 5. In some embodiments, any of the foregoing levels of identity is at least 85%, at least 90%, at least 95%, at least 98%, at least 99%, or 100%.

In any of the foregoing embodiments, an mRNA sequence encoding the polypeptide disclosed herein may comprise a nucleic acid sequence having at least 80% identity to SEQ ID NOs: 1 or 4. In some embodiments, any of the foregoing levels of identity is at least 85%, at least 90%, at least 95%, at least 98%, at least 99%, or 100%.

In any of the foregoing embodiments, the polypeptide may comprise an amino acid sequence having at least 80%, at least 90%, at least 95%, at least 98%, at least 99%, or 100% identity to SEQ ID NOs: 303, 306, 309, or 312. In some embodiments, the polypeptide disclosed herein may comprise an amino acid sequence of SEQ ID NOs: 303, 306, 309, or 312. In any of the foregoing embodiments, a nucleic acid sequence comprising an open reading frame encoding the polypeptide disclosed herein may comprise a nucleic acid sequence having at least 80%, at least 90%, at least 95%, at least 98%, at least 99%, or 100% identity to SEQ ID NOs: SEQ ID NOs: 302, 305, 308, or 311. In some embodiments, a nucleic acid sequence comprising an open reading frame encoding the polypeptide disclosed herein comprises a nucleic acid sequence of SEQ ID NOs: SEQ ID NOs: 302, 305, 308, or 311. In any of the foregoing embodiments, an mRNA sequence encoding the polypeptide disclosed herein may comprise a nucleic acid sequence having at least 80%, at least 90%, at least 95%, at least 98%, at least 99%, or 100% identity to SEQ ID NOs: 301, 304, 307, or 310. In any of the foregoing embodiments, an mRNA sequence encoding the polypeptide disclosed herein may comprise a nucleic acid sequence of SEQ ID NOs: 301, 304, 307, or 310.

In any of the foregoing embodiments, the A3A may comprise an amino acid sequence having at least 80% identity to SEQ ID NO: 40. In some embodiments, the level of identity is at least 85%, at least 87%, at least 90%, at least 95%, at least 98%, at least 99%, or 100%. In some embodiments, the A3A comprises an amino acid sequence of SEQ ID NO: 40.

In any of the foregoing embodiments, the RNA-guided nickase may comprise an amino acid sequence having at least 80%, 90%, 95%, 98%, or 99% identity to any one of SEQ ID NOs: 70, 73, or 76. In some embodiments, the level of identity is at least 85%, at least 87%, at least 90%, at least 95%, at least 98%, at least 99%, or 100%. In some embodiments, the RNA-guided nickase comprises the amino acid sequence of SEQ ID NO: 70. In some embodiments, the RNA-guided nickase comprises the amino acid sequence of SEQ ID NO: 73. In some embodiments, the RNA-guided nickase comprises the amino acid sequence of SEQ ID NO: 76.

In any of the foregoing embodiments, the A3A may comprise an amino acid sequence having at least 80% identity to SEQ ID NO: 40 and the RNA-guided nickase may comprise an amino acid sequence having at least 80%, 90%, 95%, 98%, or 99% identity to any one of SEQ ID NOs: 70, 73, or 76. In some embodiments, the A3A comprises an amino acid sequence of SEQ ID NO: 40 and the RNA-guided nickase comprises an amino acid sequence of SEQ ID NO: 70.

F. Additional Features

1. Codon-Optimization

In some embodiments, the UGI or polypeptide comprising a cytidine deaminase (e.g., an APOBEC3A deaminase) and an RNA-guided nickase is encoded by an open reading frame (ORF) comprising a codon optimized nucleic acid sequence. In some embodiment, the codon optimized nucleic acid sequence comprises minimal adenine codons and/or minimal uridine codons.

A given ORF can be reduced in uridine content or uridine dinucleotide content, for example, by using minimal uridine codons in a sufficient fraction of the ORF. For example, an amino acid sequence for the polypeptide described herein can be back-translated into an ORF sequence by converting amino acids to codons, wherein some or all of the ORF uses the exemplary minimal uridine codons shown below. In some embodiments, at least about 50%, 55%, 60%, 65%, 70%, 75%, 80%, 85%, 90%, 95%, 98%, 99%, or 100% of the codons in the ORF are codons listed in Table 1.

TABLE 1
Exemplary minimal uridine codons

| | Amino Acid | Minimal uridine codon |
|---|---|---|
| A | Alanine | GCA or GCC or GCG |
| G | Glycine | GGA or GGC or GGG |
| V | Valine | GUC or GUA or GUG |
| D | Aspartic acid | GAC |
| E | Glutamic acid | GAA or GAG |
| I | Isoleucine | AUC or AUA |
| T | Threonine | ACA or ACC or ACG |
| N | Asparagine | AAC |
| K | Lysine | AAG or AAA |
| S | Serine | AGC |
| R | Arginine | AGA or AGG |
| L | Leucine | CUG or CUA or CUC |
| P | Proline | CCG or CCA or CCC |
| H | Histidine | CAC |
| Q | Glutamine | CAG or CAA |
| F | Phenylalanine | UUC |
| Y | Tyrosine | UAC |
| C | Cysteine | UGC |
| W | Tryptophan | UGG |
| M | Methionine | AUG |

In some embodiments, the ORF may consist of a set of codons of which at least about 75%, 80%, 85%, 90%, 95%, 98%, 99%, or 100% of the codons are codons listed in Table 1.

A given ORF can be reduced in adenine content or adenine dinucleotide content, for example, by using minimal adenine codons in a sufficient fraction of the ORF. For example, an amino acid sequence for the polypeptide described herein can be back-translated into an ORF sequence by converting amino acids to codons, wherein some or all of the ORF uses the exemplary minimal adenine codons shown below. In some embodiments, at least about 50%, 55%, 60%, 65%, 70%, 75%, 80%, 85%, 90%, 95%, 98%, 99%, or 100% of the codons in the ORF are codons listed in Table 2.

TABLE 2
Exemplary minimal adenine codons

| | Amino Acid | Minimal adenine codon |
|---|---|---|
| A | Alanine | GCU or GCC or GCG |
| G | Glycine | GGU or GGC or GGG |
| V | Valine | GUC or GUU or GUG |
| D | Aspartic acid | GAC or GAU |
| E | Glutamic acid | GAG |
| I | Isoleucine | AUC or AUU |
| T | Threonine | ACU or ACC or ACG |
| N | Asparagine | AAC or AAU |
| K | Lysine | AAG |
| S | Serine | UCU or UCC or UCG |
| R | Arginine | CGU or CGC or CGG |
| L | Leucine | CUG or CUC or CUU |
| P | Proline | CCG or CCU or CCC |
| H | Histidine | CAC or CAU |
| Q | Glutamine | CAG |
| F | Phenylalanine | UUC or UUU |
| Y | Tyrosine | UAC or UAU |
| C | Cysteine | UGC or UGU |
| W | Tryptophan | UGG |
| M | Methionine | AUG |

In some embodiments, the ORF may consist of a set of codons of which at least about 75%, 80%, 85%, 90%, 95%, 98%, 99%, or 100% of the codons are codons listed in Table 2.

To the extent feasible, any of the features described above with respect to low adenine content can be combined with any of the features described above with respect to low uridine content. So too for uridine and adenine dinucleotides. Similarly, the content of uridine nucleotides and adenine dinucleotides in the ORF may be as set forth above. Similarly, the content of uridine dinucleotides and adenine nucleotides in the ORF may be as set forth above.

A given ORF can be reduced in uridine and adenine nucleotide and/or dinucleotide content, for example, by using minimal uridine and adenine codons in a sufficient fraction of the ORF. For example, an amino acid sequence for the polypeptide described herein can be back-translated into an ORF sequence by converting amino acids to codons, wherein some or all of the ORF uses the exemplary minimal uridine and adenine codons shown below. In some embodiments, at least about 50%, 55%, 60%, 65%, 70%, 75%, 80%, 85%, 90%, 95%, 98%, 99%, or 100% of the codons in the ORF are codons listed in Table 3.

TABLE 3
Exemplary minimal uridine and adenine codons

| | | Minimal uridine and |
|---|---|---|
| | Amino Acid | adenine codon |
| A | Alanine | GCC or GCG |
| G | Glycine | GGC or GGG |
| V | Valine | GUC or GUG |
| D | Aspartic acid | GAC |
| E | Glutamic acid | GAG |
| I | Isoleucine | AUC |
| T | Threonine | ACC or ACG |
| N | Asparagine | AAC |
| K | Lysine | AAG |
| S | Serine | AGC or UCC or UCG |
| R | Arginine | CGC or CGG |
| L | Leucine | CUG or CUC |
| P | Proline | CCG or CCC |
| H | Histidine | CAC |
| Q | Glutamine | CAG |
| F | Phenylalanine | UUC |
| Y | Tyrosine | UAC |
| C | Cysteine | UGC |
| W | Tryptophan | UGG |
| M | Methionine | AUG |

In some embodiments, the ORF may consist of a set of codons of which at least about 75%, 80%, 85%, 90%, 95%, 98%, 99%, or 100% of the codons are codons listed in Table 3. As can be seen in Table 3, each of the three listed serine codons contains either one A or one U. In some embodiments, uridine minimization is prioritized by using AGC codons for serine. In some embodiments, adenine minimization is prioritized by using UCC and/or UCG codons for serine.

In some embodiments, the ORF may have codons that increase translation in a mammal, such as a human. In further embodiments, the mRNA comprises an ORF having codons that increase translation in an organ, such as the liver, of the mammal, e.g., a human. In further embodiments, the ORF may have codons that increase translation in a cell type, such as a hepatocyte, of the mammal, e.g., a human. An increase in translation in a mammal, cell type, organ of a mammal, human, organ of a human, etc., can be determined relative to the extent of translation wild-type sequence of the ORF, or relative to an ORF having a codon distribution matching the codon distribution of the organism from which the ORF was derived or the organism that contains the most similar ORF at the amino acid level. Alternatively, in some embodiments, an increase in translation for a Cas9 sequence in a mammal, cell type, organ of a mammal, human, organ of a human, etc., is determined relative to translation of an ORF with the sequence of SEQ ID NO: 2 or 5 with all else equal, including any applicable point mutations, heterologous domains, and the like. In some embodiments, at least about 75%, 80%, 85%, 90%, 95%, 96%, 97%, 98%, 99%, or 100% of the codons in an ORF are codons corresponding to highly expressed tRNAs (e.g., the highest-expressed tRNA for each amino acid) in a mammal, such as a human. In some embodiments, at least 75%, 80%, 85%, 90%, 95%, 96%, 97%, 98%, 99%, or 100% of the codons in an ORF are codons corresponding to highly expressed tRNAs (e.g., the highest-expressed tRNA for each amino acid) in a mammalian organ, such as a human organ.

Alternatively, codons corresponding to highly expressed tRNAs in an organism (e.g., human) in general may be used.

Any of the foregoing approaches to codon selection can be combined with the minimal uridine and/or adenine codons shown above, e.g., by starting with the codons of Table 1, 2, or 3, and then where more than one option is available, using the codon that corresponds to a more highly-expressed tRNA, either in the organism (e.g., human) in general, or in an organ or cell type of interest (e.g., human liver or human hepatocytes).

In some embodiments, at least 75%, 80%, 85%, 90%, 95%, 96%, 97%, 98%, 99%, or 100% of the codons in an ORF are codons from a codon set shown in Table 4 (e.g., the low U 1, low A, or low A/U codon set). The codons in the low U 1, low G, low A, and low A/U sets use codons that minimize the indicated nucleotides while also using codons corresponding to highly expressed tRNAs where more than one option is available. In some embodiments, at least 75%, 80%, 85%, 90%, 95%, 96%, 97%, 98%, 99%, or 100% of the codons in an ORF are codons from the low U 1 codon set shown in Table 4. In some embodiments, at least 75%, 80%, 85%, 90%, 95%, 96%, 97%, 98%, 99%, or 100% of the codons in an ORF are codons from the low A codon set shown in Table 4. In some embodiments, at least 75%, 80%, 85%, 90%, 95%, 96%, 97%, 98%, 99%, or 100% of the codons in an ORF are codons from the low A/U codon set shown in Table 4.

TABLE 4
Exemplary Codon Sets.

| Amino | Low U | Low U | Low | Low |
|---|---|---|---|---|
| Acid | 1 | 2 | A | A/U |
| Gly | GGC | GGG | GGC | GGC |
| Glu | GAG | GAA | GAG | GAG |
| Asp | GAC | GAC | GAC | GAC |
| Val | GTG | GTA | GTG | GTG |
| Ala | GCC | GCG | GCC | GCC |
| Arg | AGA | CGA | CGG | CGG |
| Ser | AGC | AGC | TCC | AGC |
| Lys | AAG | AAA | AAG | AAG |
| Asn | AAC | AAC | AAC | AAC |
| Met | ATG | ATG | ATG | ATG |
| Ile | ATC | ATA | ATC | ATC |
| Thr | ACC | ACG | ACC | ACC |
| Trp | TGG | TGG | TGG | TGG |
| Cys | TGC | TGC | TGC | TGC |
| Tyr | TAC | TAC | TAC | TAC |
| Leu | CTG | CTA | CTG | CTG |
| Phe | TTC | TTC | TTC | TTC |
| Gln | CAG | CAA | CAG | CAG |
| His | CAC | CAC | CAC | CAC |

2. Heterologous Functional Domains; Nuclear Localization Signals (NLS)

In some embodiments, the polypeptide comprising a cytidine deaminase (e.g., A3A) and an RNA-guided nickase further comprises one or more additional heterologous functional domains (e.g., is or comprises a ternary or higher-order fusion polypeptide).

In some embodiments, the heterologous functional domain may facilitate transport of the polypeptide into the nucleus of a cell. For example, the heterologous functional domain may be a nuclear localization signal (NLS). In some embodiments, the polypeptide may be fused with 1-10 NLS(s). In some embodiments, the polypeptide may be fused with 1-5 NLS(s). In some embodiments, the polypeptide may be fused with one NLS. Where one NLS is used, the NLS may be fused at the N-terminus or the C-terminus of the polypeptide sequence. In some embodiments, the polypeptide may be fused C-terminally to at least one NLS. An NLS may also be inserted within the polypeptide sequence. In other embodiments, the polypeptide may be fused with more than one NLS. In some embodiments, the polypeptide may be fused with 2, 3, 4, or 5 NLSs. In some embodiments, the polypeptide may be fused with two NLSs. In certain circumstances, the two NLSs may be the same (e.g., two SV40 NLSs) or different. In some embodiments, the polypeptide is fused to two SV40 NLS sequences at the carboxy terminus. In some embodiments, the polypeptide may be fused with two NLSs, one at the N-terminus and one at the C-terminus. In some embodiments, the polypeptide may be fused with 3 NLSs. In some embodiments, the polypeptide may be fused with no NLS. In some embodiments, the NLS may be a monopartite sequence, such as, e.g., the SV40 NLS, PKKKRKV (SEQ ID NO: 63) or PKKKRRV (SEQ ID NO: 121). In some embodiments, the NLS may be a bipartite sequence, such as the NLS of nucleoplasmin, KRPAATKKAGQAKKKK (SEQ ID NO: 122). In a specific embodiment, a single PKKKRKV (SEQ ID NO: 63) NLS may be fused at the C-terminus of the polypeptide. One or more linkers are optionally included at the fusion site (e.g., between the polypeptide and NLS). In some embodiments, one or more NLS(s) according to any of the foregoing embodiments are present in the polypeptide in combination with one or more additional heterologous functional domains, such as any of the heterologous functional domains described below.

In some embodiments of the mRNA disclosed herein, the cytidine deaminase (e.g., A3A) is located N-terminal to the RNA-guided nickase in the polypeptide. In some embodiments of the mRNA disclosed herein, the encoded RNA-guided nickase comprises a nuclear localization signal (NLS). In some embodiments, the NLS is fused to the C-terminus of the RNA-guided nickase. In some embodiments, the NLS is fused to the C-terminus of the RNA-guided nickase via a linker. In some embodiments, the NLS is fused to the N-terminus of the RNA-guided nickase. In some embodiments, the NLS is fused to the N-terminus of the RNA-guided nickase via a linker (e.g., SEQ ID NO: 61). In some embodiments, the NLS comprises a sequence having at least 80%, 85%, 90%, or 95% identity to any one of SEQ ID NOs: 63 and 110-122. In some embodiments, the NLS comprises the sequence of any one of SEQ ID NOs: 63 and 110-122. In some embodiments, the NLS is encoded by a sequence having at least 80%, 85%, 90%, 95%, 98% or 100% identity to the sequence of any one of SEQ ID NOs: 63 and 110-122.

In some embodiments, the heterologous functional domain may be capable of modifying the intracellular half-life of the A3A and/or the RNA-guided nickase in the polypeptide. In some embodiments, the half-life of the A3A and/or the RNA-guided nickase in the polypeptide may be increased. In some embodiments, the half-life of the A3A and/or the RNA-guided nickase in the polypeptide may be reduced. In some embodiments, the heterologous functional domain may be capable of increasing the stability of the A3A and/or the RNA-guided nickase in the polypeptide. In some embodiments, the heterologous functional domain may be capable of reducing the stability of the A3A and/or the RNA-guided nickase in the polypeptide. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the polypeptide may be modified by addition of ubiquitin or a polyubiquitin chain. In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called Rub1 in S. cerevisiae), human leukocyte antigen F-associated (FAT10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBL5).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Any known fluorescent proteins may be used as the marker domain such as GFP, YFP, EBFP, ECFP, DsRed or any other suitable fluorescent protein. In some embodiments, the marker domain may be a purification tag and/or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His, 8×His, biotin carboxyl carrier protein (BCCP), poly-His, and calmodulin. In some embodiments, the marker domain may be a reporter gene. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the heterologous functional domain may target the polypeptide to a specific organelle, cell type, tissue, or organ. In some embodiments, the heterologous functional domain may target the polypeptide to mitochondria.

3. UTRs; Kozak Sequences

In some embodiments, the nucleic acid (e.g., mRNA) disclosed herein comprises a 5′ UTR, 3′ UTR, or 5′ and 3′ UTRs from Hydroxysteroid 17-Beta Dehydrogenase 4 (HSD17B4 or HSD) or globin such as human alpha globin (HBA), human beta globin (HBB), Xenopus laevis beta globin (XBG), bovine growth hormone, cytomegalovirus (CMV), mouse Hba-al, heat shock protein 90 (Hsp90), glyceraldehyde 3-phosphate dehydrogenase (GAPDH), beta-actin, alpha-tubulin, tumor protein (p53), or epidermal growth factor receptor (EGFR).

In some embodiments, the nucleic acid disclosed herein comprises a 5′ UTR from HSD and a 3′ UTR from a human albumin gene. In some embodiments, an mRNA disclosed herein comprises a 5′ UTR with at least 90% identity to any one of SEQ ID NO: 93 and a 3′ UTR with at least 90% identity to any one of SEQ ID NO: 69.

In some embodiments, the nucleic acid disclosed herein comprises a 5′ UTR with at least 90% identity to any one of SEQ ID NOs: 91-98. In some embodiments, an mRNA disclosed herein comprises a 3′ UTR with at least 90% identity to any one of SEQ ID NOs: 69, 99-106. In some embodiments, any of the foregoing levels of identity is at least 95%, at least 98%, at least 99%, or 100%. In some embodiments, an mRNA disclosed herein comprises a 5′ UTR having the sequence of any one of SEQ ID NOs: 91-98. In some embodiments, an mRNA disclosed herein comprises a 3′ UTR having the sequence of any one of SEQ ID NOs: 69, 99-106. In some embodiments, the mRNA comprises a 5′ UTR and a 3′ UTR from the same source.

In some embodiments, the nucleic acid described herein does not comprise a 5′ UTR, e.g., there are no additional nucleotides between the 5′ cap and the start codon. In some embodiments, the mRNA comprises a Kozak sequence (described below) between the 5′ cap and the start codon, but does not have any additional 5′ UTR. In some embodiments, the mRNA does not comprise a 3′ UTR, e.g., there are no additional nucleotides between the stop codon and the poly-A tail.

In some embodiments, the nucleic acid herein comprises a Kozak sequence. The Kozak sequence can affect translation initiation and the overall yield of a polypeptide translated from an mRNA. A Kozak sequence includes a methionine codon that can function as the start codon. A minimal Kozak sequence is NNNRUGN wherein at least one of the following is true: the first N is A or G and the second N is G. In the context of a nucleotide sequence, R means a purine (A or G). In some embodiments, the Kozak sequence is RNNRUGN, NNNRUGG, RNNRUGG, RNNAUGN, NNNAUGG, RNNAUGG, or GCCACCAUG. In some embodiments, the Kozak sequence is rccRUGg, rccAUGg, gccAccAUG, gccRccAUGG (SEQ ID NO: 107) or gccgccRccAUGG (SEQ ID NO: 108), with zero mismatches or with up to one or two mismatches to positions in lowercase.

4. Poly-A Tail

In some embodiments, the nucleic acid disclosed herein further comprises a poly-adenylated (poly-A) tail. The poly-A tails may comprise at least 8 consecutive adenine nucleotides, but also comprise one or more non-adenine nucleotide. As used herein, “non-adenine nucleotides” refer to any natural or non-natural nucleotides that do not comprise adenine. Guanine, thymine, and cytosine nucleotides are exemplary non-adenine nucleotides. Thus, the poly-A tails on the nucleic acid described herein may comprise consecutive adenine nucleotides located 3′ to nucleotides encoding a polypeptide of interest. In some instances, the poly-A tails on mRNA comprise non-consecutive adenine nucleotides located 3′ to nucleotides encoding a polypeptide comprising a cytidine deaminase (e.g., A3A) and an RNA-guided nickase or a sequence of interest, wherein non-adenine nucleotides interrupt the adenine nucleotides at regular or irregularly spaced intervals.

In some embodiments, the poly-A tail is encoded in the plasmid used for in vitro transcription of mRNA and becomes part of the transcript. The poly-A sequence encoded in the plasmid, i.e., the number of consecutive adenine nucleotides in the poly-A sequence, may not be exact, e.g., a 100 poly-A sequence in the plasmid may not result in a precisely 100 poly-A sequence in the transcribed mRNA. In some embodiments, the poly-A tail is not encoded in the plasmid, and is added by PCR tailing or enzymatic tailing, e.g., using E. coli poly(A) polymerase.

In some embodiments, the one or more non-adenine nucleotides are positioned to interrupt the consecutive adenine nucleotides so that a poly(A) binding protein can bind to a stretch of consecutive adenine nucleotides. In some embodiments, one or more non-adenine nucleotide(s) is located after at least 8, 9, 10, 11, or 12 consecutive adenine nucleotides. In some embodiments, the one or more non-adenine nucleotide is located after 8-50 consecutive adenine nucleotides. In some embodiments, the one or more non-adenine nucleotide is located after 8-100 consecutive adenine nucleotides.

In some embodiments, the poly-A tail comprises or contains one non-adenine nucleotide or one consecutive stretch of 2-10 non-adenine nucleotides.

In some embodiments, the non-adenine nucleotide is guanine, cytosine, or thymine. In some instances, where more than one non-adenine nucleotide is present, the non-adenine nucleotide may be selected from: a) guanine and thymine nucleotides; b) guanine and cytosine nucleotides; c) thymine and cytosine nucleotides; or d) guanine, thymine and cytosine nucleotides. An exemplary poly-A tail comprising non-adenine nucleotides is provided as SEQ ID NO: 109.

5. Modified Nucleotides

In some embodiments, the nucleic acid disclosed herein comprises a modified uridine at some or all uridine positions. In some embodiments, the modified uridine is a uridine modified at the 5 position, e.g., with a halogen or C1-C3 alkoxy. In some embodiments, the modified uridine is a pseudouridine modified at the 1 position, e.g., with a C1-C3 alkyl. The modified uridine can be, for example, pseudouridine, N1-methyl-pseudouridine, 5-methoxyuridine, 5-iodouridine, or a combination thereof.

In some embodiments, at least 10%, 15%, 20%, 25%, 30%, 35%, 40%, 45%, 50%, 55%, 60%, 65%, 70%, 75%, 80%, 85%, 90%, 95%, 98%, 99%, or 100% of the uridine positions in the nucleic acid disclosed herein are modified uridines. In some embodiments, 10%-25%, 15-25%, 25-35%, 35-45%, 45-55%, 55-65%, 65-75%, 75-85%, 85-95%, or 90-100% of the uridine positions in an mRNA disclosed herein are modified uridines, e.g., 5-methoxyuridine, 5-iodouridine, N1-methyl pseudouridine, pseudouridine, or a combination thereof.

In some embodiments, at least 10% of the uridine is substituted with a modified uridine. In some embodiments, 15% to 45% of the uridine is substituted with the modified uridine. In some embodiments, at least 20%, at least 30%, at least 40%, at least 50%, at least 60%, at least 70%, at least 80%, at least 90%, or 100% of the uridine is substituted with the modified uridine.

6. 5′ Cap

In some embodiments, the nucleic acid disclosed herein comprises a 5′ cap, such as a Cap0, Cap1, or Cap2. A 5′ cap is generally a 7-methylguanine ribonucleotide (which may be further modified, as discussed below e.g., with respect to ARCA) linked through a 5′-triphosphate to the 5′ position of the first nucleotide of the 5′-to-3′ chain of the nucleic acid, i.e., the first cap-proximal nucleotide. In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc Natl Acad Sci USA 111(33):12025-30; Abbas et al. (2017) Proc Natl Acad Sci USA 114(11):E2106-E2115. Most endogenous higher eukaryotic nucleic acids, including mammalian nucleic acids such as human nucleic acids, comprise Cap1 or Cap2. Cap0 and other cap structures differing from Cap1 and Cap2 may be immunogenic in mammals, such as humans, due to recognition as “non-self” by components of the innate immune system such as IFIT-1 and IFIT-5, which can result in elevated cytokine levels including type I interferon. Components of the innate immune system such as IFIT-1 and IFIT-5 may also compete with eIF4E for binding of a nucleic acids with a cap other than Cap1 or Cap2, potentially inhibiting translation of the nucleic acid.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap or a Cap0-like cap in which the 2′ position of the first cap-proximal nucleotide is hydroxyl. See, e.g., Stepinski et al., (2001) “Synthesis and properties of mRNAs containing the novel ‘anti-reverse’ cap analogs 7-methyl(3′-O-methyl)GpppG and 7-methyl(3′deoxy)GpppG,” RNA 7: 1486-1495. The ARCA structure is shown below.

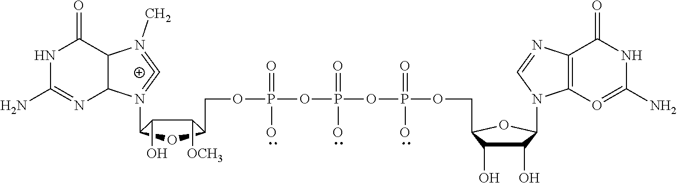

CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively. The CleanCap™ AG structure is shown below. CleanCap™ structures are sometimes referred to herein using the last three digits of the catalog numbers listed above (e.g., “CleanCap™ 113” for TriLink Biotechnologies Cat. No. N-7113).

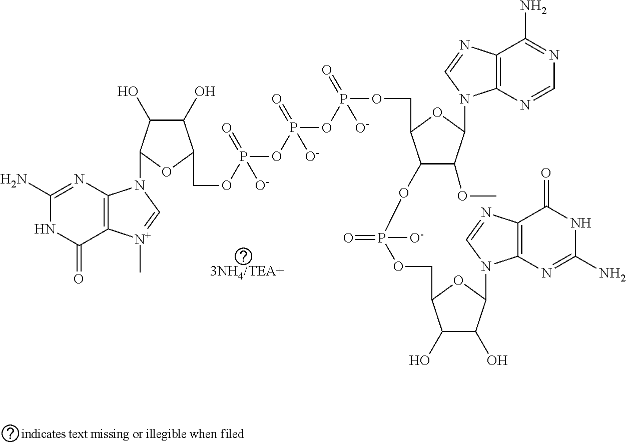 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21]

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0, in the presence of S-adenosyl methionine and GTP. See, e.g., Guo, P. and Moss, B. (1990) Proc. Natl. Acad. Sci. USA 87, 4023-4027; Mao, X. and Shuman, S. (1994) J. Biol. Chem. 269, 24472-24479. For additional discussion of caps and capping approaches, see, e.g., WO2017/053297 and Ishikawa et al., Nucl. Acids. Symp. Ser. (2009) No. 53, 129-130.

G. Guide RNA (gRNA)

In some embodiments, the compositions comprise at least one guide RNA (gRNA), and the methods comprise delivering at least one gRNA, wherein the gRNA directs the editor to a desired genomic location. In some embodiments, a composition comprises an mRNA described herein and at least one gRNA. In some embodiments, a composition comprises a polypeptide described herein and at least one gRNA. In some embodiments, the gRNA is a single guide RNA (sgRNA). In some embodiments, the gRNA is a dual guide RNA (dgRNA).

A gRNA disclosed herein may comprise a guide sequence that directs a polypeptide comprising a cytidine deaminase (e.g., an APOBEC3A deaminase) and an RNA-guided nickase to a cytosine (C) located in any region of a gene (e.g., within the coding region of a gene) for cytosine (C) to thymine (T) conversion (“C-to-T conversion”).

In some embodiments, the C-to-T conversion alters a DNA sequence, such as a human genetic sequence. In some embodiments, the C-to-T conversion alters the coding sequence of a gene. In some embodiments, the C-to-T conversion generates a stop codon, for example, a premature stop codon within the coding region of a gene. In some embodiments, the C-to-T conversion eliminates a stop codon. In some embodiments, the C-to-T conversion alters the regulatory sequence of a gene (e.g., a gene promotor or gene repressor). In some embodiments, the C-to-T conversion alters the splicing of a gene. In some embodiments, the C-to-T conversion corrects a genetic defect associated with a disease or disorder.

In some embodiments, a guide RNA (gRNA) comprises a guide sequence that directs a polypeptide comprising a cytidine deaminase (e.g., an APOBEC3A deaminase) and an RNA-guided nickase to a splice donor or acceptor site in a gene. In some embodiments, the splice donor or acceptor is a splice donor site. In some embodiments, the splice donor or acceptor site is a splice acceptor site.

In some embodiments, a guide RNA (gRNA) comprises a guide sequence that directs a polypeptide comprising a cytidine deaminase (e.g., an APOBEC3A deaminase) and an RNA-guided nickase to an acceptor splice site boundary. In some embodiments, a guide RNA (gRNA) comprises a guide sequence that directs a polypeptide comprising a cytidine deaminase (e.g., A3A) and an RNA-guided nickase to a donor splice site boundary.

In some embodiments, a guide RNA (gRNA) comprises a guide sequence that directs a polypeptide comprising a cytidine deaminase (e.g., an APOBEC3A deaminase) and an RNA-guided nickase to make a single-strand cut in a gene at a cut site 3′ of an acceptor splice site boundary or 5′ of an acceptor splice site boundary. In this and the following discussion, 3′ and 5′ indicate directions in the sense of the strand being cut.

In some embodiments, a guide RNA (gRNA) disclosed herein comprises a guide sequence that directs a polypeptide comprising a cytidine deaminase (e.g., an APOBEC3A deaminase) and an RNA-guided nickase for making a single-strand cut in a gene at a cut site that is 3′ of a donor splice site boundary or 5′ of a donor splice site boundary.

A “splice site,” as used herein, refers to the three nucleotides that make up an acceptor splice site or a donor splice site (defined below), or any other nucleotides known in the art that are part of a splice site. See e.g., Burset et al., Nucleic Acids Research 28(21):4364-4375 (2000) (describing canonical and non-canonical splice sites in mammalian genomes). The three nucleotides that make up an “acceptor splice site” are two conserved residues (e.g., AG in humans) at the 3′ of an intron and a boundary nucleotide (i.e., the first nucleotide of the exon 3′ of the AG). The three nucleotides that make up a “donor splice site” are two conserved residues (e.g., GT (gene) or GU (in RNA such as pre-mRNA) in human) at the 5′ end of an intron and a boundary nucleotide (i.e., the first nucleotide of the exon 5′ of the GT).

In some embodiments, a composition comprising at least one gRNA is provided in combination with a nucleic acid (e.g., an mRNA) disclosed herein. In some embodiments, one or more gRNA is provided as a separate molecule from the nucleic acid (e.g., an mRNA) disclosed herein. In some embodiments, a gRNA is provided as a part, such as a part of a UTR, of the nucleic acid disclosed herein.

In some embodiments, a composition is provided comprising a polypeptide comprising a cytidine deaminase and an RNA-guided nickase, and a gRNA. In some embodiments, a ribonucleoprotein complex (RNP) is provided, the RNP comprising a polypeptide comprising a cytidine deaminase and an RNA-guided nickase and a gRNA. In some embodiments, the polypeptide does not comprise a UGI.

The gRNA comprises a guide sequence targeting a particular gene or genetic sequence. In some embodiments, the gRNA is a Cas nickase guide. In some embodiments, the gRNA is a Class 2 Cas nickase guide. In further embodiments, the gRNA is a Cpf1 or Cas9 guide. In some embodiments, the gRNA is a Nme nickase guide. In some embodiments, the Nme nickase is a Nme1, Nme2, or Nme3 nickase. In some embodiments, the gRNA comprises a guide sequence 5′ of an RNA that forms two or more hairpin or stem-loop structures. CRISPR/Cas gRNA structures are known in the art and vary with their cognate Cas nuclease. In general, the gRNA used together with any particular Cas9 or Nme nickase described herein must function with that nickase. For example, when the polypeptide disclosed herein comprises a SpyCas9 nickase, the gRNA provided is a SpyCas9 guide RNA (as described herein). When the polypeptide disclosed herein comprises a NmeCas9 nickase, the guide RNA is a NmeCas9 guide RNA (as described herein).

In some embodiments, the gRNA comprises a guide sequence that direct an RNA-guided nickase (e.g., Cas9 nickase), to a target DNA sequence in a target locus, such as a target gene. Targets and exemplary target sequences targeting each gene are exemplified herein and include, but are not limited to, targets and guide sequences disclosed in e.g., WO2017185054 (for trinucleotide repeats in transcription factor four (TCF4)); WO 2018119182 A1 (targeting SERPINA1); WO 2019/067872 (targeting transthyretin (7TR); WO 2020/028327 A1 (targeting hydroxyacid oxidase 1 (HA01), the contents of each of which are hereby incorporated by reference in their entirety. One skilled in the art will be familiar with suitable guide sequences for targeting other genes or loci of interest.

The gRNA may comprise a crRNA comprising 17, 18, 19, 20, 21, 22, 23, 24, or 25 contiguous nucleotides of a guide sequence. The gRNA may further comprise a trRNA. In each composition and method embodiment described herein, the crRNA and trRNA may be associated as a single RNA (sgRNA), or may be on separate RNAs (dgRNA). In the context of sgRNAs, the crRNA and trRNA components may be covalently linked, e.g., via a phosphodiester bond or other covalent bond.

In each of the composition, use, and method embodiments described herein, the gRNA may comprise two RNA molecules as a “dual guide RNA” or “dgRNA”. The dgRNA comprises a first RNA molecule comprising a crRNA comprising a guide sequence, and a second RNA molecule comprising a trRNA. The first and second RNA molecules may not be covalently linked, but may form a RNA duplex via the base pairing between portions of the crRNA and the trRNA.

In each of the composition, use, and method embodiments described herein, the gRNA may comprise a single RNA molecule as a “single guide RNA” or “sgRNA”. The sgRNA may comprise a crRNA (or a portion thereof) comprising a guide sequence covalently linked to, e.g., a trRNA. The sgRNA may comprise 17, 18, 19, 20, 21, 22, 23, 24, or 25 contiguous nucleotides of a guide sequence. In some embodiments, the crRNA and the trRNA are covalently linked via a linker. In some embodiments, the sgRNA forms a stem-loop structure via the base pairing between portions of the crRNA and the trRNA. In some embodiments, the crRNA and the trRNA are covalently linked via one or more bonds that are not a phosphodiester bond.

In some embodiments, the trRNA may comprise all or a portion of a trRNA sequence derived from a naturally-occurring CRISPR/Cas system. In some embodiments, the trRNA comprises a truncated or modified wild type trRNA. The length of the trRNA depends on the CRISPR/Cas system used. In some embodiments, the trRNA comprises or consists of 5, 6, 7, 8, 9, 10, 11, 12, 13, 14, 15, 16, 17, 18, 19, 20, 25, 30, 40, 50, 60, 70, 80, 90, 100, or more than 100 nucleotides. In some embodiments, the trRNA may comprise certain secondary structures, such as, for example, one or more hairpin or stem-loop structures, or one or more bulge structures.

The gRNAs provided herein can be useful for recognizing (e.g., hybridizing to) a target sequence in the gene. In some embodiments, the selection of the one or more gRNAs is determined based on target sequences within the gene. In some embodiments, a gRNA complementary or having complementarity to a target sequence within the target locus is used to direct a polypeptide comprising a cytidine deaminase (e.g., A3A) and an RNA-guided nickase to a particular location in the locus. The target locus may be recognized and nicked by a Cas nickase comprising a gRNA.

In some embodiments, the guide sequence is at least 99%, 98%, 97%, 96%, 95%, 94%, 93%, 92%, 91%, or 90% identical to a target sequence. In some embodiments, the target sequence may be complementary to the guide sequence of the gRNA. In some embodiments, the degree of complementarity or identity between a guide sequence of a gRNA and its corresponding target sequence may be about 50%, 55%, 60%, 65%, 70%, 75%, 80%, 85%, 90%, 95%, 96%, 97%, 98%, 99%, or 100%. In some embodiments, the target sequence and the guide sequence of the gRNA may be 100% complementary or identical. In other embodiments, the target sequence and the guide sequence of the gRNA may contain at least one mismatch. For example, the target sequence and the guide sequence of the gRNA may contain 1, 2, 3, 4, 5, 6, 7, 8, 9, or 10 mismatches, where the total length of the target sequence is at least about 17, 18, 19, 20 or more base pairs. In some embodiments, the target sequence and the guide sequence of the gRNA may contain 1, 2, 3, 4, 5, or 6 mismatches where the guide sequence is 20 nucleotides.

The gRNA may comprise a guide sequence linked to additional nucleotides to form a crRNA, e.g., with the following exemplary nucleotide sequence following the guide sequence at its 3′ end: GUUUUAGAGCUAUGCUGUUUUG (SEQ ID NO: 139).

In the case of an sgRNA, the guide sequence may be linked to additional nucleotides to form a sgRNA, e.g., with the following exemplary nucleotide sequence following the 3′ end of the guide sequence: GUUUUAGAGCUAGAAAUAGCAAGUUAAAAUAAGGCUAGUCCGUUAUCAACUU GAAAAAGUGGCACCGAGUCGGUGCUUUU (SEQ ID NO: 140) in 5′ to 3′ orientation.

In some embodiments, the sgRNA comprises the modification pattern shown below in SEQ ID NO: 141, where N is any natural or non-natural nucleotide, and where the totality of the N's comprise a guide sequence as described herein and the modified sgRNA comprises the following sequence: mN*mN*mN*NNNNNNNNNNNNNNNNNGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 141), where “N” may be any natural or non-natural nucleotide. For example, encompassed herein is SEQ ID NO: 141, where the N's are replaced with any of the guide sequences disclosed herein. The modifications remain as shown in SEQ ID NO: 141 despite the substitution of N's for the nucleotides of a guide. That is, although the nucleotides of the guide replace the “N's”, the first three nucleotides are 2′OMe modified and there are phosphorothioate linkages between the first and second nucleotides, the second and third nucleotides and the third and fourth nucleotides.

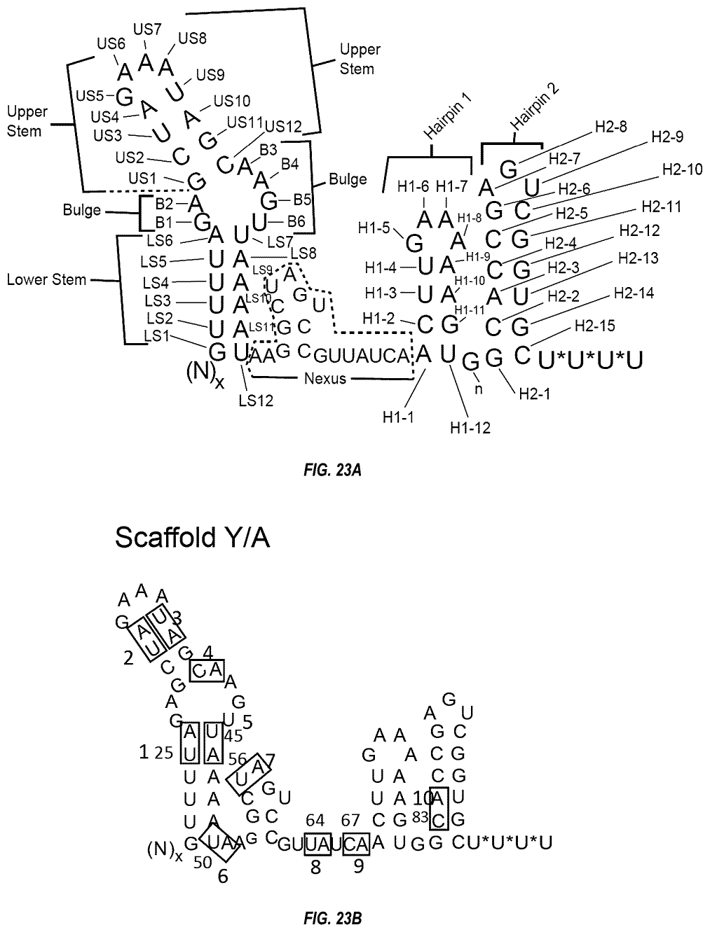

FIG. 23A shows an exemplary sgRNA (SEQ ID NO: 141, methylation not shown) in a possible secondary structure with labels designating individual nucleotides of the conserved region of the sgRNA, including the lower stem, bulge, upper stem, nexus (the nucleotides of which can be referred to as N1 through N18, respectively, in the 5′ to 3′ direction), and the hairpin region which includes hairpin 1 and hairpin 2 regions. A nucleotide between hairpin 1 and hairpin 2 is labeled n. A guide region may be present on an sgRNA and is indicated in this figure as “(N)x” preceding the conserved region of the sgRNA. In some embodiments, the sgRNA may further comprise one or more nucleotides between the lower stem and bulge regions, between the bulge and the upper stem region, between the upper stem and the nexus, or the between the nexus and the hairpin 1 region or between the hairpin 1 and hairpin 2 regions.

In some embodiments, a conserved portion of the sgRNA is a conserved region of a spyCas9 or a spyCas9 equivalent. In some embodiments, a conserved portion of the sgRNA is not from S. pyogenes Cas9, such as Staphylococcus aureus Cas9 (“saCas9”). Further description of regions of exemplary sgRNAs are provided in WO2019/237069 published Dec. 12, 2019, the entire contents of which are incorporated herein by reference.

The SpyCas9 gRNA may comprise internal linkers. In some embodiments, the internal linker may have a bridging length of about 3-30, optionally 12-21 atoms, and the linker substitutes for at least 2 nucleotides of the gRNA. In some embodiments, the internal linker has a bridging length of about 6-18 atoms, optionally about 6-12 atoms, and the linker substitutes for at least 2 nucleotides of the gRNA.

In some embodiments the internal linker comprises at least two ethylene glycol subunits covalently linked to each other. In some embodiments, the internal linker comprises a PEG-linker.

In some embodiments, the internal linker comprises a PEG-linker having from 1 to 10 ethylene glycol units. In some embodiments, the internal linker comprises a PEG-linker having from 3 to 6 ethylene glycol units. In some embodiments, the internal linker comprises a PEG-linker having 3 ethylene glycol units. In some embodiments, the internal linker comprises a PEG-linker having 6 ethylene glycol units.

In some embodiments, the conserved portion of a spyCas9 guide RNA comprises a repeat-anti-repeat region, a hairpin 1 region, and a hairpin 2 region, and further comprises at least one of:
1) a first internal linker substituting for at least 2 nucleotides of an upper stem region of the repeat-anti-repeat region of the sgRNA;
2) a second internal linker substituting for 1 or 2 nucleotides of the hairpin 1 of the sgRNA; or
3) a third internal linker substituting for at least 2 nucleotides of the hairpin 2 of the sgRNA.

Exemplary locations of the linkers in the spyCas9 guide RNA are as shown in the following: NNNNNNNNNNNNNNNNNNNNGUUUUAGAGCUA(L1)UAGCAAGUUAAAAUAAG GCUAGUCCGUUAUCAACUU(L1)AAGUGGCACCGAGUCGGUGCUUUUU (SEQ ID NO: 524) where N are nucleotides encoding a guide sequence.

As used herein, “Linker 1” or “L1” refers to an internal linker having a bridging length of about 15-21 atoms. As used herein, “Linker 2” or “L2” refers to an internal linker having a bridging length of about 6-12 atoms.

In some embodiments, the spyCas9 guide RNA comprising internal linkers may be chemically modified. Exemplary modifications include a modification pattern of the following sequence:

(SEQ ID NO: 523)
mA*mC*mG*CAAAUAUCAGUCCAGCGGUUUUAGAmGmCmUmA(L1)mUm
AmGmCAAGUUAAAAUAAGGC(L2)GUCCGUUAUCAC(L1)GGGCACCGA
GUCGG*mU*mG*mC

In some embodiments, the gRNA comprises a 3′ tail. In some embodiments, the 3′ tail consists of a nucleotide comprising a uracil or modified uracil. In some embodiments, the 3′ terminal nucleotide is a modified nucleotide. In some embodiments, wherein the 3′ tail comprises a modification of any one or more of the nucleotides present in the 3′ tail. In further embodiments, wherein the modification of the 3′ tail is one or more of 2′-O-methyl (2′-OMe) modified nucleotide and a phosphorothioate (PS) linkage between nucleotides. penultimate nucleotide.

1. Short-Single Guide RNA (Short-sgRNA)

In some embodiments, an sgRNA provided herein is a short-single guide RNAs (short-sgRNAs), e.g., comprising a conserved portion of an sgRNA comprising a hairpin region, wherein the hairpin region lacks at least 5-10 nucleotides or 6-10 nucleotides. In some embodiments, the 5-10 nucleotides or 6-10 nucleotides are consecutive.

In some embodiments, a short-sgRNA lacks at least nucleotides 54-58 (AAAAA) of the conserved portion of a spyCas9 sgRNA. In some embodiments, a short-sgRNA is a non-spyCas9 sgRNA that lacks nucleotides corresponding to nucleotides 54-58 (AAAAA) of the conserved portion of a spyCas9 as determined, for example, by pairwise or structural alignment.

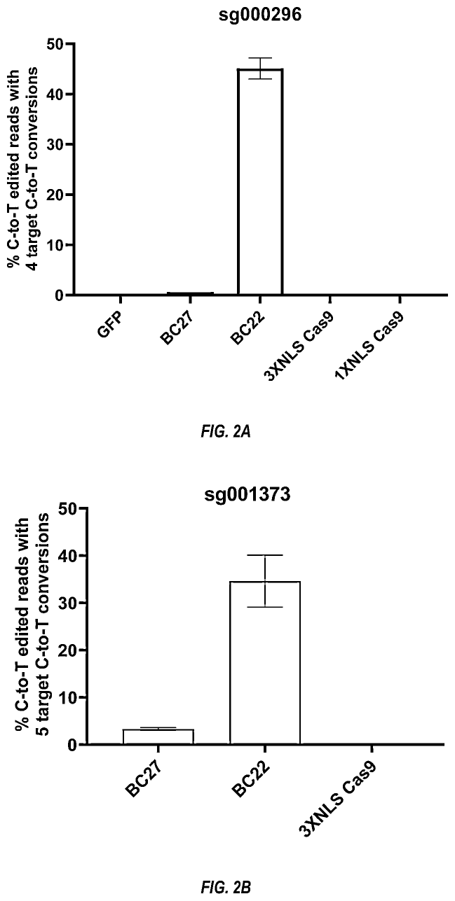

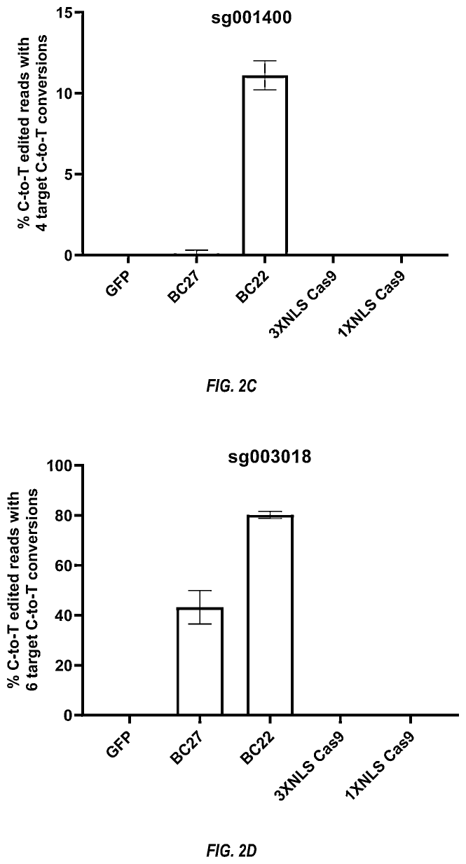

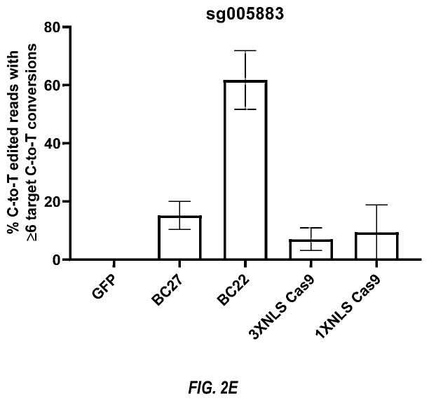

Structural alignment is useful where molecules share similar structures despite considerable sequence variation. Structural alignment involves identifying corresponding residues across two (or more) sequences by (i) modeling the structure of a first sequence using the known structure of the second sequence or (ii) comparing the structures of the first and second sequences where both are known, and identifying the residue in the first sequence most similarly positioned to a residue of interest in the second sequence. Corresponding residues are identified in some algorithms based on distance minimization given position (e.g., nucleobase position 1 or the 1′ carbon of the pentose ring for polynucleotides, or alpha carbons for polypeptides) in the overlaid structures (e.g., what set of paired positions provides a minimized root-mean-square deviation for the alignment). When identifying positions in a non-spyCas9 gRNA corresponding to positions described with respect to spyCas9 gRNA, spyCas9 gRNA can be the “second” sequence. Where a non-spyCas9 gRNA of interest does not have an available known structure, but is more closely related to another non-spyCas9 gRNA that does have a known structure, it may be most effective to model the non-spyCas9 gRNA of interest using the known structure of the closely related non-spyCas9 gRNA, and then compare that model to the spyCas9 gRNA structure to identify the desired corresponding residue in the non-spyCas9 gRNA of interest. There is an extensive literature on structural modeling and alignment for proteins; representative disclosures include U.S. Pat. Nos. 6,859,736; 8,738,343; and those cited in Aslam et al., Electronic Journal of Biotechnology 20 (2016) 9-13. For discussion of modeling a structure based on a known related structure or structures, see, e.g., Bordoli et al., Nature Protocols 4 (2009) 1-13, and references cited therein. See also FIG. 2(F) from Nishimasu et al., Cell 162(5): 1113-1126 (2015) for alignment of nucleic acid.

In some embodiments, the short-sgRNA described herein comprises a conserved portion comprising a hairpin region, wherein the hairpin region lacks 5, 6, 7, 8, 9, 10, 11, or 12 nucleotides. In some embodiments, the lacking nucleotides are 5-10 lacking nucleotides or 6-10 lacking nucleotides. In some embodiments, the lacking nucleotides are consecutive. In some embodiments, the lacking nucleotides span at least a portion of hairpin 1 and a portion of hairpin 2. In some embodiments, the 5-10 lacking nucleotides comprise or consist of nucleotides 54-58, 54-61, or 53-60 of SEQ ID NO: 140.

In some embodiments, the short-sgRNA described herein further comprises a nexus region, wherein the nexus region lacks at least one nucleotide (e.g., 1, 2, 3, 4, 5, 6, 7, 8, 9, or 10 nucleotides in the nexus region). In some embodiments, the short-sgRNA lacks each nucleotide in the nexus region.

In some embodiments, the SpyCas9 short-sgRNA described herein comprises a sequence of NNNNNNNNNNNNNNNNNNNNGUUUUAGAGCUAGAAAUAGCAAGUUAAAAUAA GGCUAGUCCGUUAUCACGAAAGGGCACCGAGUCGGUGCU (SEQ ID NO: 521). In some embodiments, the short-sgRNA described herein comprises a modification pattern as shown in SEQ ID NO: 520: mN*mN*mN*GUUUUAGAmGmCmUmAmGmAmAmAmUmAmGmCAAGUUAAAAUA AGGCUAGUCCGUUAUCACGAAAGGGCACCGAGUCGGmUmGmC*mU (SEQ ID NO: 520), where A, C, G, U, and N are adenine, cytosine, guanine, uracil, and any ribonucleotide, respectively, unless otherwise indicated. An m is indicative of a 2′O-methyl modification, and an * is indicative of a phosphorothioate linkage between the nucleotides.

In some embodiments, a gRNA described herein is an N. meningitidis Cas9 (NmeCas9) gRNA comprising a conserved portion comprising a repeat/anti-repeat region, a hairpin 1 region, and a hairpin 2 region, wherein one or more of the repeat/anti-repeat region, the hairpin 1 region, and the hairpin 2 region are shortened. Exemplary wild-type NmeCas9 guide RNA comprises a sequence of (N)₂₀₋₂₅ GUUGUAGCUCCCUUUCUCAUUUCGGAAACGAAAUGAGAACCGUUGCUACAAU AAGGCCGUCUGAAAAGAUGUGCCGCAACGCUCUGCCCCUUAAAGCUUCUGCUU UAAGGGGCAUCGUUUA (SEQ ID NO: 512). (N)₂₀-25 as used herein represent 20-25, i.e., 20, 21, 22, 23, 24, or 25 consecutive N. A, C, G, and U represent nucleotides having adenine, cytosine, guanine, and uracil bases, respectively. In some embodiments, (N)₂₀₋₂₅ has 24 nucleotides in length. N is any natural or non-natural nucleotide, and where the totality of the N's comprises a guide sequence.

In some embodiments, the conserved portion of the NmeCas9 short-gRNA comprises:
(a) a shortened repeat/anti-repeat region, wherein the shortened repeat/anti-repeat region lacks 2-24 nucleotides, wherein
   (i) one or more of nucleotides 37-48 and 53-64 is deleted and optionally one or more of nucleotides 37-64 is substituted relative to SEQ ID NO: 512; and
   (ii) nucleotide 36 is linked to nucleotide 65 by at least 2 nucleotides; or
b) a shortened hairpin 1 region, wherein the shortened hairpin 1 lacks 2-10, optionally 2-8 nucleotides, wherein
   (i) one or more of nucleotides 82-86 and 91-95 is deleted and optionally one or more of positions 82-96 is substituted relative to SEQ ID NO: 512 and
   (ii) nucleotide 81 is linked to nucleotide 96 by at least 4 nucleotides; or
(c) a shortened hairpin 2 region, wherein the shortened hairpin 2 lacks 2-18, optionally 2-16 nucleotides, wherein
   (i) one or more of nucleotides 113-121 and 126-134 is deleted and optionally one or more of nucleotides 113-134 is substituted relative to SEQ ID NO: 512; and
   (ii) nucleotide 112 is linked to nucleotide 135 by at least 4 nucleotides; wherein one or both nucleotides 144-145 are optionally deleted relative to SEQ ID NO: 512; and wherein at least 10 nucleotides are modified nucleotides.

In some embodiments, the NmeCas9 short-gRNA comprises one of the following sequences in 5′ to 3′ orientation:

(SEQ ID NO: 513)
(N)₂₀₋₂₅GUUGUAGCUCCCUGAAACCGUUGCUACAAUAAGGCCGUCGAAA
GAUGUGCCGCAACGCUCUGCCUUCUGGCAUCGUU;
(SEQ ID NO: 514)
(N)₂₀₋₂₅GUUGUAGCUCCCUGAAACCGUUGCUACAAUAAGGCCGUCGAAA
GAUGUGCCGCAACGCUCUGCCUUCUGGCAUCGUUUAUU;
(SEQ ID NO: 515)
(N)₂₀₋₂₅GUUGUAGCUCCCUGGAAACCCGUUGCUACAAUAAGGCCGUCGA
AAGAUGUGCCGCAACGCUCUGCCUUCUGGCAUCGUUUAUU

In some embodiments, at least 10 nucleotides of the conserved portion of the NmeCas9 short-sgRNA are modified nucleotides.

In some embodiments, the NmeCas9 short-sgRNA comprises a conserved region comprising one of the following sequences in 5′ to 3′ orientation: GUUGmUmAmGmCUCCCmUmGmAmAmAmCmCGUUmGmCUAmCAAU*AAGmGm CCmGmUmCmGmAmAmAmGmAmUGUGCmCGCmAmAmCmGCUCUmGmCCmUmU mCmUGmGCmAmUC*mG*mU*mU (SEQ ID NO: 516); or GUUGmUmAmGmCUCCCmUmGmAmAmAmCmCGUUmGmCUAmCAAU*AAGmGm CCmGmUmCmGmAmAmAmGmAmUGUGCmCGmCAAmCGCUCUmGmCCmUmUmC mUGGCAUCG*mU*mU (SEQ ID NO: 517).

The shortened NmeCas9 gRNA may comprise internal linkers disclosed herein.

“Internal linker” as used herein describes a non-nucleotide segment joining two nucleotides within a guide RNA. If the gRNA contains a spacer region, the internal linker is located outside of the spacer region (e.g., in the scaffold or conserved region of the gRNA). For Type V guides, it is understood that the last hairpin is the only hairpin in the structure, i.e., the repeat-anti-repeat region. In some embodiments, the internal linker comprises a PEG-linker disclosed herein.

Exemplary locations of the linkers are as shown in the following: (N)₂₀₋₂₅ GUUGUAGCUCCCUUC(L1)GACCGUUGCUACAAUAAGGCCGUC(L1)GAUGU GCCGCAACGCUCUGCC(L1)GGCAUCGUU (SEQ ID NO: 518). As used herein, (L1) refers to an internal linker having a bridging length of about 15-21 atoms.

In some embodiments, the shortened NmeCas9 guide RNA comprising internal linkers may be chemically modified. Exemplary modifications include a modification pattern of the following sequence: mN*mN*mN*mNmNNNmNmNNmNNmNNNNNmNNNNmNNNmGUUGmUmAmGmC UCCCmUmUmC(L1)mGmAmCmCGUUmGmCUAmCAAU*AAGmGmCCmGmUmC(L1) mGmAmUGUGCmCGmCAAmCGCUCUmGmCC(L1)GGCAUCG*mU*mU (SEQ ID NO: 519).

2. Modifications

In some embodiments, the gRNA (e.g., sgRNA, short-sgRNA, dgRNA, or crRNA) is modified. The term “modified” or “modification” in the context of a gRNA described herein includes, the modifications described above, including, for example, (a) end modifications, e.g., 5′ end modifications or 3′ end modifications, including 5′ or 3′ protective end modifications, (b) nucleobase (or “base”) modifications, including replacement or removal of bases, (c) sugar modifications, including modifications at the 2′, 3′, and/or 4′ positions, (d) internucleoside linkage modifications, and (e) backbone modifications, which can include modification or replacement of the phosphodiester linkages and/or the ribose sugar. A modification of a nucleotide at a given position includes a modification or replacement of the phosphodiester linkage immediately 3′ of the sugar of the nucleotide. Thus, for example, a nucleic acid comprising a phosphorothioate between the first and second sugars from the 5′ end is considered to comprise a modification at position 1. The term “modified gRNA” generally refers to a gRNA having a modification to the chemical structure of one or more of the base, the sugar, and the phosphodiester linkage or backbone portions, including nucleotide phosphates, all as detailed and exemplified herein (see the modification patterns shown in e.g., SEQ ID NOs: 142-145, 181-185 and 191-203).

Further description and exemplary patterns of modifications are provided in in Table 1 of WO2019/237069 published Dec. 12, 2019, the entire contents of which are incorporated herein by reference.

In some embodiments, a gRNA comprises modifications at 1, 2, 3, 4, 5, 6, 7, 8, 9, 10, 11, 12, 13, 14, 15, 16, or more YA sites. In some embodiments, the pyrimidine of the YA site comprises a modification (which includes a modification altering the internucleoside linkage immediately 3′ of the sugar of the pyrimidine). In some embodiments, the adenine of the YA site comprises a modification (which includes a modification altering the internucleoside linkage immediately 3′ of the sugar of the adenine). In some embodiments, the pyrimidine and the adenine of the YA site comprise modifications, such as sugar, base, or internucleoside linkage modifications. The YA modifications can be any of the types of modifications set forth herein. In some embodiments, the YA modifications comprise one or more of phosphorothioate, 2′-OMe, or 2′-fluoro. In some embodiments, the YA modifications comprise pyrimidine modifications comprising one or more of phosphorothioate, 2′-OMe, 2′-H, inosine, or 2′-fluoro. In some embodiments, the YA modification comprises a bicyclic ribose analog (e.g., an LNA, BNA, or ENA) within an RNA duplex region that contains one or more YA sites. In some embodiments, the YA modification comprises a bicyclic ribose analog (e.g., an LNA, BNA, or ENA) within an RNA duplex region that contains a YA site, wherein the YA modification is distal to the YA site.

In some embodiments, the guide sequence (or guide region) of a gRNA comprises 1, 2, 3, 4, 5, or more YA sites (“guide region YA sites”) that may comprise YA modifications. In some embodiments, one or more YA sites located at 5-end, 6-end, 7-end, 8-end, 9-end, or 10-end from the 5′ end of the 5′ terminus (where “5-end”, etc., refers to position 5 to the 3′ end of the guide region, i.e., the most 3′ nucleotide in the guide region) comprise YA modifications. A modified guide region YA site comprises a YA modification.

In some embodiments, a modified guide region YA site is within 20, 19, 18, 17, 16, 15, 14, 13, 12, 11, 10, or 9 nucleotides of the 3′ terminal nucleotide of the guide region. For example, if a modified guide region YA site is within 10 nucleotides of the 3′ terminal nucleotide of the guide region and the guide region is 20 nucleotides long, then the modified nucleotide of the modified guide region YA site is located at any of positions 11-20. In some embodiments, a modified guide region YA site is at or after nucleotide 4, 5, 6, 7, 8, 9, 10, or 11 from the 5′ end of the 5′ terminus.

In some embodiments, a modified guide region YA site is other than a 5′ end modification. For example, a sgRNA can comprise a 5′ end modification as described herein and further comprise a modified guide region YA site. Alternatively, a sgRNA can comprise an unmodified 5′ end and a modified guide region YA site. Alternatively, a short-sgRNA can comprise a modified 5′ end and an unmodified guide region YA site.

In some embodiments, a modified guide region YA site comprises a modification that at least one nucleotide located 5′ of the guide region YA site does not comprise. For example, if nucleotides 1-3 comprise phosphorothioates, nucleotide 4 comprises only a 2′-OMe modification, and nucleotide 5 is the pyrimidine of a YA site and comprises a phosphorothioate, then the modified guide region YA site comprises a modification (phosphorothioate) that at least one nucleotide located 5′ of the guide region YA site (nucleotide 4) does not comprise. In another example, if nucleotides 1-3 comprise phosphorothioates, and nucleotide 4 is the pyrimidine of a YA site and comprises a 2′-OMe, then the modified guide region YA site comprises a modification (2′-OMe) that at least one nucleotide located 5′ of the guide region YA site (any of nucleotides 1-3) does not comprise. This condition is also always satisfied if an unmodified nucleotide is located 5′ of the modified guide region YA site.

In some embodiments, the modified guide region YA sites comprise modifications as described for YA sites above. The guide region of a gRNA may be modified according to any embodiment comprising a modified guide region set forth herein.

Conserved region YA sites 1-10 are illustrated in FIG. 23B. In some embodiments, 1, 2, 3, 4, 5, 6, 7, 8, 9, or 10 conserved region YA sites comprise modifications. In some embodiments, conserved region YA sites 1, 8, or 1 and 8 comprise YA modifications. In some embodiments, conserved region YA sites 1, 2, 3, 4, and 10 comprise YA modifications. In some embodiments, YA sites 2, 3, 4, 8, and 10 comprise YA modifications. In some embodiments, conserved region YA sites 1, 2, 3, and 10 comprise YA modifications. In some embodiments, YA sites 2, 3, 8, and 10 comprise YA modifications. In some embodiments, YA sites 1, 2, 3, 4, 8, and 10 comprise YA modifications. In some embodiments, 1, 2, 3, 4, 5, 6, 7, or 8 additional conserved region YA sites comprise YA modifications.

In some embodiments, the modified conserved region YA sites comprise modifications as described for YA sites above. Any embodiments set forth elsewhere in this disclosure may be combined to the extent feasible with any of the foregoing embodiments.

In some embodiments, the 5′ and/or 3′ terminus regions of a gRNA are modified.

In some embodiments, the terminal (i.e., last) 1, 2, 3, 4, 5, 6, or 7 nucleotides in the 3′ terminus region are modified. Throughout, this modification may be referred to as a “3′ end modification”. In some embodiments, the terminal (i.e., last) 1, 2, 3, 4, 5, 6, or 7 nucleotides in the 3′ terminus region comprise more than one modification. In some embodiments, the 3′ end modification comprises or further comprises any one or more of the following: a modified nucleotide selected from 2′-O-methyl (2′-O-Me) modified nucleotide, 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or combinations thereof. In some embodiments, the 3′ end modification comprises or further comprises modifications of 1, 2, 3, 4, 5, 6, or 7 nucleotides at the 3′ end of the gRNA. In some embodiments, the 3′ end modification comprises or further comprises one PS linkage, wherein the linkage is between the last and second to last nucleotide. In some embodiments, the 3′ end modification comprises or further comprises two PS linkages between the last three nucleotides. In some embodiments, the 3′ end modification comprises or further comprises four PS linkages between the last four nucleotides. In some embodiments, the 3′ end modification comprises or further comprises PS linkages between any one or more of the last 2, 3, 4, 5, 6, or 7 nucleotides. In some embodiments, the gRNA comprising a 3′ end modification comprises or further comprises a 3′ tail, wherein the 3′ tail comprises a modification of any one or more of the nucleotides present in the 3′ tail. In some embodiments, the 3′ tail is fully modified. In some embodiments, the 3′ tail comprises 1, 2, 3, 4, 5, 6, 7, 8, 9, 10, 1-2, 1-3, 1-4, 1-5, 1-6, 1-7, 1-8, 1-9, or 1-10 nucleotides, optionally where any one or more of these nucleotides are modified. In some embodiments, a gRNA is provided comprising a 3′ end modification, wherein the 3′ end modification comprises the 3′ end modification as shown in any one of SEQ ID Nos: 141-145. In some embodiments, a gRNA is provided comprising a 3′ protective end modification. In some embodiments, the 3′ tail comprises between 1 and about 20 nucleotides, between 1 and about 15 nucleotides, between 1 and about 10 nucleotides, between 1 and about 5 nucleotides, between 1 and about 4 nucleotides, between 1 and about 3 nucleotides, and between 1 and about 2 nucleotides. In some embodiments, the gRNA does not comprise a 3′ tail.

In some embodiments, the 5′ terminus region is modified, for example, the first 1, 2, 3, 4, 5, 6, or 7 nucleotides of the gRNA are modified. Throughout, this modification may be referred to as a “5′ end modification”. In some embodiments, the first 1, 2, 3, 4, 5, 6, or 7 nucleotides of the 5′ terminus region comprise more than one modification. In some embodiments, at least one of the terminal (i.e., first) 1, 2, 3, 4, 5, 6, or 7 nucleotides at the 5′ end are modified. In some embodiments, both the 5′ and 3′ terminus regions (e.g., ends) of the gRNA are modified. In some embodiments, only the 5′ terminus region of the gRNA is modified. In some embodiments, only the 3′ terminus region (plus or minus a 3′ tail) of the conserved portion of a gRNA is modified. In some embodiments, the gRNA comprises modifications at 1, 2, 3, 4, 5, 6, or 7 of the first 7 nucleotides at a 5′ terminus region of the gRNA. In some embodiments, the gRNA comprises modifications at 1, 2, 3, 4, 5, 6, or 7 of the 7 terminal nucleotides at a 3′ terminus region. In some embodiments, 2, 3, or 4 of the first 4 nucleotides at the 5′ terminus region, and/or 2, 3, or 4 of the terminal 4 nucleotides at the 3′ terminus region are modified. In some embodiments, 2, 3, or 4 of the first 4 nucleotides at the 5′ terminus region are linked with phosphorothioate (PS) bonds. In some embodiments, the modification to the 5′ terminus and/or 3′ terminus comprises a 2′-O-methyl (2′-O-Me) or 2′-O-(2-methoxyethyl) (2′-O-moe) modification. In some embodiments, the modification comprises a 2′-fluoro (2′-F) modification to a nucleotide. In some embodiments, the modification comprises a phosphorothioate (PS) linkage between nucleotides. In some embodiments, the modification comprises an inverted abasic nucleotide. In some embodiments, the modification comprises a protective end modification. In some embodiments, the modification comprises a more than one modification selected from protective end modification, 2′-O-Me, 2′-O-moe, 2′-fluoro (2′-F), a phosphorothioate (PS) linkage between nucleotides, and an inverted abasic nucleotide. In some embodiments, an equivalent modification is encompassed. In some embodiments, a gRNA is provided comprising a 5′ end modification, wherein the 5′ end modification comprises a 5′ end modification as shown in any one of SEQ ID Nos: 141-145.

In some embodiments, a gRNA is provided comprising a 5′ end modification and a 3′ end modification. In some embodiments, the gRNA comprises modified nucleotides that are not at the 5′ or 3′ ends.

In some embodiments, a sgRNA is provided comprising an upper stem modification, wherein the upper stem modification comprises a modification to any one or more of US1-US12 in the upper stem region. In some embodiments, a sgRNA is provided comprising an upper stem modification, wherein the upper stem modification comprises a modification of at least 1, 2, 3, 4, 5, 6, 7, 8, 9, 10, 11, or all 12 nucleotides in the upper stem region. In some embodiments, an sgRNA is provided comprising an upper stem modification, wherein the upper stem modification comprises 1, 2, 3, 4, or 5 YA modifications in a YA site. In some embodiments, the upper stem modification comprises a 2′-OMe modified nucleotide, a 2′-O-moe modified nucleotide, a 2′-F modified nucleotide, and/or combinations thereof. Other modifications described herein, such as a 5′ end modification and/or a 3′ end modification may be combined with an upper stem modification.

In some embodiments, the sgRNA comprises a modification in the hairpin region. In some embodiments, the hairpin region modification comprises at least one modified nucleotide selected from a 2′-O-methyl (2′-OMe) modified nucleotide, a 2′-fluoro (2′-F) modified nucleotide, and/or combinations thereof. In some embodiments, the hairpin region modification is in the hairpin 1 region. In some embodiments, the hairpin region modification is in the hairpin 2 region. In some embodiments, the hairpin modification comprises 1, 2, or 3 YA modifications in a YA site. In some embodiments, the hairpin modification comprises at least 1, 2, 3, 4, 5, or 6 YA modifications. Other modifications described herein, such as an upper stem modification, a 5′ end modification, and/or a 3′ end modification may be combined with a modification in the hairpin region.

In some embodiments, a gRNA comprises a substituted and optionally shortened hairpin 1 region, wherein at least one of the following pairs of nucleotides are substituted in the substituted and optionally shortened hairpin 1 with Watson-Crick pairing nucleotides: H1-1 and H1-12, H1-2 and H1-11, H1-3 and H1-10, and/or H1-4 and H1-9. “Watson-Crick pairing nucleotides” include any pair capable of forming a Watson-Crick base pair, including A-T, A-U, T-A, U-A, C-G, and G-C pairs, and pairs including modified versions of any of the foregoing nucleotides that have the same base pairing preference. In some embodiments, the hairpin 1 region lacks any one or two of H1-5 through H1-8. In some embodiments, the hairpin 1 region lacks one, two, or three of the following pairs of nucleotides: H1-1 and H1-12, H1-2 and H1-11, H1-3 and H1-10 and/or H1-4 and H1-9. In some embodiments, the hairpin 1 region lacks 1-8 nucleotides of the hairpin 1 region. In any of the foregoing embodiments, the lacking nucleotides may be such that the one or more nucleotide pairs substituted with Watson-Crick pairing nucleotides (H1-1 and H1-12, H1-2 and H1-11, H1-3 and H1-10, and/or H1-4 and H1-9) form a base pair in the gRNA.

In some embodiments, the gRNA further comprises an upper stem region lacking at least 1 nucleotide, e.g., any of the shortened upper stem regions indicated in Table 7 of U.S. Application No. 62/946,905, the contents of which are hereby incorporated by reference in its entirety, or described elsewhere herein, which may be combined with any of the shortened or substituted hairpin 1 regions described herein.

In some embodiments, the gRNA described herein further comprises a nexus region, wherein the nexus region lacks at least one nucleotide.

3. Chemical Modifications of gRNAs

In some embodiments, the gRNA is chemically modified. A gRNA comprising one or more modified nucleosides or nucleotides is called a “modified” gRNA or “chemically modified” gRNA, to describe the presence of one or more non-naturally and/or naturally occurring components or configurations that are used instead of or in addition to the canonical A, G, C, and U residues. Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification).

Chemical modifications such as those listed above can be combined to provide modified gRNAs comprising nucleosides and nucleotides (collectively “residues”) that can have two, three, four, or more modifications. For example, a modified residue can have a modified sugar and a modified nucleobase. In some embodiments, every base of a gRNA is modified, e.g., all bases have a modified phosphate group, such as a phosphorothioate group. In certain embodiments, all, or substantially all, of the phosphate groups of an gRNA molecule are replaced with phosphorothioate groups. In some embodiments, modified gRNAs comprise at least one modified residue at or near the 5′ end of the RNA. In some embodiments, modified gRNAs comprise at least one modified residue at or near the 3′ end of the RNA.

In some embodiments, the gRNA comprises one, two, three or more modified residues. In some embodiments, at least 5% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) of the positions in a modified gRNA are modified nucleosides or nucleotides.

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include phosphorothioate, phosphoroselenates, borano phosphates, borano phosphate esters, hydrogen phosphonates, phosphoroamidates, alkyl or aryl phosphonates and phosphotriesters.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e. at sugar modification. For example, the 2′ hydroxyl group (OH) can be modified, e.g. replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion. Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20. In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges. In some embodiments, the 2′ hydroxyl group modification can included “unlocked” nucleic acids (UNA) in which the ribose ring lacks the C2′-C3′ bond. In some embodiments, the 2′ hydroxyl group modification can include the methoxyethyl group (MOE), (OCH₂CH₂OCH₃, e.g., a PEG derivative).

“Deoxy” 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH2; alkylamino, dialkylamino, heterocyclyl, arylamino, diarylamino, heteroarylamino, diheteroarylamino, or amino acid); NH(CH₂CH₂NH)ₙCH2CH₂— amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA and/or tracr RNA. In embodiments comprising an sgRNA, one or more residues at one or both ends of the sgRNA may be chemically modified, or the entire sgRNA may be chemically modified. Certain embodiments comprise a 5′ end modification. Certain embodiments comprise a 3′ end modification. In certain embodiments, one or more or all of the nucleotides in single stranded overhang of a gRNA molecule are deoxynucleotides.

In some embodiments, the gRNAs disclosed herein comprise one of the modification patterns disclosed in WO2018/107028 A1, published Jun. 14, 2018 the contents of which are hereby incorporated by reference in their entirety.

The terms “mA,” “mC,” “mU,” or “mG” may be used to denote a nucleotide that has been modified with 2′-O-Me. The terms “fA,” “fC,” “fU,” or “fU” may be used to denote a nucleotide that has been substituted with 2′-F. A “*” may be used to depict a PS modification. The terms A*, C*, U*, or G* may be used to denote a nucleotide that is linked to the next (e.g., 3′) nucleotide with a PS bond. The terms “mA*,” “mC*,” “mU*,” or “mG*” may be used to denote a nucleotide that has been substituted with 2′-O-Me and that is linked to the next (e.g., 3′) nucleotide with a PS bond.

H. Lipids; Formulation; Delivery

Disclosed herein are various embodiments using lipid nucleic acid assembly compositions comprising nucleic acids(s), or composition(s) described herein. In some embodiments, the lipid nucleic acid assembly composition comprises a nucleic acid (e.g., mRNA) comprising an open reading frame encoding a first polypeptide comprising a cytidine deaminase (e.g., A3A) and an RNA-guided nickase. In some embodiments, the lipid nucleic acid assembly composition comprises a first nucleic acid comprising an open reading frame encoding a first polypeptide comprising a cytidine deaminase (e.g., A3A) and an RNA-guided nickase and a second nucleic acid encoding a UGI.

As used herein, a “lipid nucleic acid assembly composition” refers to lipid-based delivery compositions, including lipid nanoparticles (LNPs) and lipoplexes. LNP refers to lipid nanoparticles <100 nM. LNPs are formed by precise mixing a lipid component (e.g., in ethanol) with an aqueous nucleic acid component and LNPs are uniform in size. Lipoplexes are particles formed by bulk mixing the lipid and nucleic acid components and are between about 100 nm and 1 micron in size. In certain embodiments the lipid nucleic acid assemblies are LNPs. As used herein, a “lipid nucleic acid assembly” comprises a plurality of (i.e. more than one) lipid molecules physically associated with each other by intermolecular forces. A lipid nucleic acid assembly may comprise a bioavailable lipid having a pKa value of <7.5 or <7. The lipid nucleic acid assemblies are formed by mixing an aqueous nucleic acid-containing solution with an organic solvent-based lipid solution, e.g., 100% ethanol. Suitable solutions or solvents include or may contain: water, PBS, Tris buffer, NaCl, citrate buffer, ethanol, chloroform, diethylether, cyclohexane, tetrahydrofuran, methanol, isopropanol. A pharmaceutically acceptable buffer may optionally be comprised in a pharmaceutical formulation comprising the lipid nucleic acid assemblies, e.g., for an ex vivo therapy. In some embodiments, the aqueous solution comprises an RNA, such as an mRNA or a gRNA. In some embodiments, the aqueous solution comprises an mRNA encoding an RNA-guided DNA binding agent, such as Cas9.

As used herein, lipid nanoparticle (LNP) refers to a particle that comprises a plurality of (i.e., more than one) lipid molecules physically associated with each other by intermolecular forces. The LNPs may be, e.g., microspheres (including unilamellar and multilamellar vesicles, e.g., “liposomes”—lamellar phase lipid bilayers that, in some embodiments, are substantially spherical—and, in more particular embodiments, can comprise an aqueous core, e.g., comprising a substantial portion of RNA molecules), a dispersed phase in an emulsion, micelles, or an internal phase in a suspension. Emulsions, micelles, and suspensions may be suitable compositions for local and/or topical delivery. See also, e.g., WO2017173054A1, the contents of which are hereby incorporated by reference in their entirety. Any LNP known to those of skill in the art to be capable of delivering nucleotides to subjects may be utilized with the guide RNAs and the nucleic acid encoding an RNA-guided nickase and the nucleic acid encoding a cytidine deaminase described herein.

In some embodiments, the aqueous solution comprises a nucleic acid encoding a polypeptide comprising an A3A and an RNA-guided nickase. A pharmaceutical formulation comprising the lipid nucleic acid assembly composition may optionally comprise a pharmaceutically acceptable buffer.

In some embodiments, the lipid nucleic acid assembly compositions include an “amine lipid” (sometimes herein or elsewhere described as an “ionizable lipid” or a “biodegradable lipid”), together with an optional “helper lipid”, a “neutral lipid”, and a stealth lipid such as a PEG lipid. In some embodiments, the amine lipids or ionizable lipids are cationic depending on the pH.

1. Amine Lipids

In some embodiments, lipid nucleic acid assembly compositions comprise an “amine lipid”, which is, for example an ionizable lipid such as Lipid A or its equivalents, including acetal analogs of Lipid A.

In some embodiments, the amine lipid is Lipid A, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. Lipid A can be depicted as:

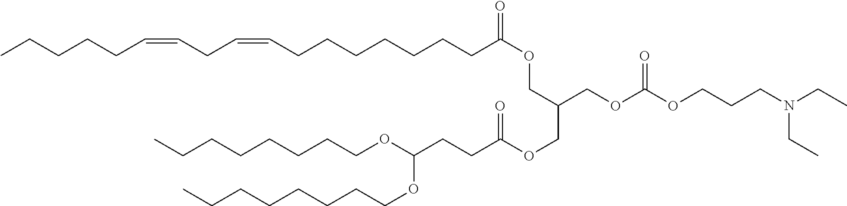 [SMILES: CCCCC/C=C\C/C=C\CCCCCCCC(=O)OCC(COC(=O)CCC(OCCCCCCCC)OCCCCCCCC)COC(=O)OCCCN(CC)CC]

Lipid A may be synthesized according to WO2015/095340 (e.g., pp. 84-86). In some embodiments, the amine lipid is an equivalent to Lipid A.

In some embodiments, an amine lipid is an analog of Lipid A. In some embodiments, a Lipid A analog is an acetal analog of Lipid A. In particular lipid nucleic acid assembly compositions, the acetal analog is a C4-C12 acetal analog. In some embodiments, the acetal analog is a C5-C12 acetal analog. In additional embodiments, the acetal analog is a C5-C10 acetal analog. In further embodiments, the acetal analog is chosen from a C4, C5, C6, C7, C9, C10, C11, and C12 acetal analog.

Amine lipids and other “biodegradable lipids” suitable for use in the lipid nucleic acid assemblies described herein are biodegradable in vivo or ex vivo. The amine lipids have low toxicity (e.g., are tolerated in animal models without adverse effect in amounts of greater than or equal to 10 mg/kg). In some embodiments, lipid nucleic acid assemblies comprising an amine lipid include those where at least 75% of the amine lipid is cleared from the plasma or the engineered cell within 8, 10, 12, 24, or 48 hours, or 3, 4, 5, 6, 7, or 10 days. In some embodiments, lipid nucleic acid assemblies comprising an amine lipid include those where at least 50% of the nucleic acid, e.g., mRNA or gRNA, is cleared from the plasma within 8, 10, 12, 24, or 48 hours, or 3, 4, 5, 6, 7, or 10 days. In some embodiments, lipid nucleic acid assemblies comprising an amine lipid include those where at least 50% of the lipid nucleic acid assembly is cleared from the plasma within 8, 10, 12, 24, or 48 hours, or 3, 4, 5, 6, 7, or 10 days, for example by measuring a lipid (e.g. an amine lipid), nucleic acid, e.g., RNA/mRNA, or other component. In some embodiments, lipid-encapsulated versus free lipid, RNA, or nucleic acid component of the lipid nucleic acid assembly is measured.

Biodegradable lipids include, for example the biodegradable lipids of WO/2020/219876, WO/2020/118041, WO/2020/072605, WO/2019/067992, WO/2017/173054, WO2015/095340, and WO2014/136086, and LNPs include LNP compositions described therein, the lipids and compositions of which are hereby incorporated by reference.

Lipid clearance may be measured as described in literature. See Maier, M. A., et al. Biodegradable Lipids Enabling Rapidly Eliminated Lipid Nanoparticles for Systemic Delivery of RNAi Therapeutics. Mol. Ther. 2013, 21(8), 1570-78 (“Maier”). For example, in Maier, LNP-siRNA systems containing luciferases-targeting siRNA were administered to six- to eight-week old male C57Bl/6 mice at 0.3 mg/kg by intravenous bolus injection via the lateral tail vein. Blood, liver, and spleen samples were collected at 0.083, 0.25, 0.5, 1, 2, 4, 8, 24, 48, 96, and 168 hours post-dose. Mice were perfused with saline before tissue collection and blood samples were processed to obtain plasma. All samples were processed and analyzed by LC-MS. Further, Maier describes a procedure for assessing toxicity after administration of LNP-siRNA formulations. For example, a luciferase-targeting siRNA was administered at 0, 1, 3, 5, and 10 mg/kg (5 animals/group) via single intravenous bolus injection at a dose volume of 5 mL/kg to male Sprague-Dawley rats. After 24 hours, about 1 mL of blood was obtained from the jugular vein of conscious animals and the serum was isolated. At 72 hours post-dose, all animals were euthanized for necropsy. Assessments of clinical signs, body weight, serum chemistry, organ weights and histopathology were performed. Although Maier describes methods for assessing siRNA-LNP formulations, these methods may be applied to assess clearance, pharmacokinetics, and toxicity of administration of lipid nucleic acid assembly compositions of the present disclosure.

Ionizable and bioavailable lipids for LNP delivery of nucleic acids known in the art are suitable. Lipids may be ionizable depending upon the pH of the medium they are in. For example, in a slightly acidic medium, the lipid, such as an amine lipid, may be protonated and thus bear a positive charge. Conversely, in a slightly basic medium, such as, for example, blood where pH is approximately 7.35, the lipid, such as an amine lipid, may not be protonated and thus bear no charge.

The ability of a lipid to bear a charge is related to its intrinsic pKa. In some embodiments, the amine lipids of the present disclosure may each, independently, have a pKa in the range of from about 5.1 to about 7.4. In some embodiments, the bioavailable lipids of the present disclosure may each, independently, have a pKa in the range of from about 5.1 to about 7.4, such as from about 5.5 to about 6.6, from about 5.6 to about 6.4, from about 5.8 to about 6.2, or from about 5.8 to about 6.5. For example, the amine lipids of the present disclosure may each, independently, have a pKa in the range of from about 5.8 to about 6.5. Lipids with a pKa ranging from about 5.1 to about 7.4 are effective for delivery of cargo in vivo, e.g. to the liver. Further, it has been found that lipids with a pKa ranging from about 5.3 to about 6.4 are effective for delivery in vivo, e.g. to tumors. See, e.g., WO2014/136086.

2. Additional Lipids

“Neutral lipids” suitable for use in a lipid nucleic acid assembly composition of the disclosure include, for example, a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine (DSPC), pohsphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In one embodiment, the neutral phospholipid may be selected from the group consisting of distearoylphosphatidylcholine (DSPC) and dimyristoyl phosphatidyl ethanolamine (DMPE). In another embodiment, the neutral phospholipid may be distearoylphosphatidylcholine (DSPC).

“Helper lipids” include steroids, sterols, and alkyl resorcinols. Helper lipids suitable for use in the present disclosure include, but are not limited to, cholesterol, 5-heptadecylresorcinol, and cholesterol hemisuccinate. In one embodiment, the helper lipid may be cholesterol. In one embodiment, the helper lipid may be cholesterol hemisuccinate.

“Stealth lipids” are lipids that alter the length of time the nanoparticles can exist in vivo (e.g., in the blood). Stealth lipids may assist in the formulation process by, for example, reducing particle aggregation and controlling particle size. Stealth lipids used herein may modulate pharmacokinetic properties of the lipid nucleic acid assembly or aid in stability of the nanoparticle ex vivo. Stealth lipids suitable for use in a lipid nucleic acid assembly composition of the disclosure include, but are not limited to, stealth lipids having a hydrophilic head group linked to a lipid moiety. Stealth lipids suitable for use in a lipid nucleic acid assembly composition of the present disclosure and information about the biochemistry of such lipids can be found in Romberg et al., Pharmaceutical Research, Vol. 25, No. 1, 2008, pg. 55-71 and Hoekstra et al., Biochimica et Biophysica Acta 1660 (2004) 41-52. Additional suitable PEG lipids are disclosed, e.g., in WO 2006/007712.

In one embodiment, the hydrophilic head group of stealth lipid comprises a polymer moiety selected from polymers based on PEG. Stealth lipids may comprise a lipid moiety. In some embodiments, the stealth lipid is a PEG lipid.

In one embodiment, a stealth lipid comprises a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids and poly[N-(2-hydroxypropyl)methacrylamide].

In one embodiment, the PEG lipid comprises a polymer moiety based on PEG (sometimes referred to as poly(ethylene oxide)).

The PEG lipid further comprises a lipid moiety. In some embodiments, the lipid moiety may be derived from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. In some embodiments, the alkyl chain length comprises about C10 to C20. The dialkylglycerol or dialkylglycamide group can further comprise one or more substituted alkyl groups. The chain lengths may be symmetrical or asymmetrical.

Unless otherwise indicated, the term “PEG” as used herein means any polyethylene glycol or other polyalkylene ether polymer. In one embodiment, PEG is an optionally substituted linear or branched polymer of ethylene glycol or ethylene oxide. In one embodiment, PEG is unsubstituted. In one embodiment, the PEG is substituted, e.g., by one or more alkyl, alkoxy, acyl, hydroxy, or aryl groups. In one embodiment, the term includes PEG copolymers such as PEG-polyurethane or PEG-polypropylene (see, e.g., J. Milton Harris, Poly(ethylene glycol) chemistry: biotechnical and biomedical applications (1992)); in another embodiment, the term does not include PEG copolymers. In one embodiment, the PEG has a molecular weight of from about 130 to about 50,000, in a sub-embodiment, about 150 to about 30,000, in a sub-embodiment, about 150 to about 20,000, in a sub-embodiment about 150 to about 15,000, in a sub-embodiment, about 150 to about 10,000, in a sub-embodiment, about 150 to about 6,000, in a sub-embodiment, about 150 to about 5,000, in a sub-embodiment, about 150 to about 4,000, in a sub-embodiment, about 150 to about 3,000, in a sub-embodiment, about 300 to about 3,000, in a sub-embodiment, about 1,000 to about 3,000, and in a sub-embodiment, about 1,500 to about 2,500.

In some embodiments, the PEG (e.g., conjugated to a lipid moiety or lipid, such as a stealth lipid), is a “PEG-2K,” also termed “PEG 2000,” which has an average molecular weight of about 2,000 daltons. PEG-2K is represented herein by the following formula (I), wherein n is 45, meaning that the number averaged degree of polymerization comprises about 45 subunits

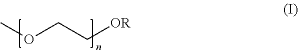

However, other PEG embodiments known in the art may be used, including, e.g., those where the number-averaged degree of polymerization comprises about 23 subunits (n=23), and/or 68 subunits (n=68). In some embodiments, n may range from about 30 to about 60. In some embodiments, n may range from about 35 to about 55. In some embodiments, n may range from about 40 to about 50. In some embodiments, n may range from about 42 to about 48. In some embodiments, n may be 45. In some embodiments, R may be selected from H, substituted alkyl, and unsubstituted alkyl. In some embodiments, R may be unsubstituted alkyl. In some embodiments, R may be methyl.

In any of the embodiments described herein, the PEG lipid may be selected from PEG-dilauroylglycerol, PEG-dimyristoylglycerol (PEG-DMG) (catalog #GM-020 from NOF, Tokyo, Japan), PEG-dipalmitoylglycerol, PEG-distearoylglycerol (PEG-DSPE) (catalog #DSPE-020CN, NOF, Tokyo, Japan), PEG-dilaurylglycamide, PEG-dimyristylglycamide, PEG-dipalmitoylglycamide, and PEG-distearoylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000] (PEG2k-DMG) (cat. #880150P from Avanti Polar Lipids, Alabaster, Alabama, USA), 1,2-distearoyl-sn-glycero-3-phosphoethanolamine-N-[methoxy(polyethylene glycol)-2000] (PEG2k-DSPE) (cat. #880120C from Avanti Polar Lipids, Alabaster, Alabama, USA), 1,2-distearoyl-sn-glycerol, methoxypolyethylene glycol (PEG2k-DSG; GS-020, NOF Tokyo, Japan), poly(ethylene glycol)-2000-dimethacrylate (PEG2k-DMA), and 1,2-distearyloxypropyl-3-amine-N-[methoxy(polyethylene glycol)-2000] (PEG2k-DSA). In one embodiment, the PEG lipid may be PEG2k-DMG. In some embodiments, the PEG lipid may be PEG2k-DSG. In one embodiment, the PEG lipid may be PEG2k-DSPE. In one embodiment, the PEG lipid may be PEG2k-DMA. In one embodiment, the PEG lipid may be PEG2k-C-DMA. In one embodiment, the PEG lipid may be compound S027, disclosed in WO2016/010840 (paragraphs [00240] to [00244]). In one embodiment, the PEG lipid may be PEG2k-DSA. In one embodiment, the PEG lipid may be PEG2k-C11. In some embodiments, the PEG lipid may be PEG2k-C14. In some embodiments, the PEG lipid may be PEG2k-C16. In some embodiments, the PEG lipid may be PEG2k-C18.

3. Formulations

The lipid nucleic acid assembly may contain (i) a biodegradable lipid, (ii) an optional neutral lipid, (iii) a helper lipid, and (iv) a stealth lipid, such as a PEG lipid. The lipid nucleic acid assembly may contain a biodegradable lipid and one or more of a neutral lipid, a helper lipid, and a stealth lipid, such as a PEG lipid.

The lipid nucleic acid assembly may contain (i) an amine lipid for encapsulation and for endosomal escape, (ii) a neutral lipid for stabilization, (iii) a helper lipid, also for stabilization, and (iv) a stealth lipid, such as a PEG lipid. The lipid nucleic acid assembly may contain an amine lipid and one or more of a neutral lipid, a helper lipid, also for stabilization, and a stealth lipid, such as a PEG lipid.

The mRNAs required to achieve the described functional effects described herein may be delivered to a cell in one or more lipid nucleic acid assembly composition(s). For example, one lipid nucleic acid assembly composition may be formulated for delivery comprising mRNA encoding a polypeptide comprising a cytidine deaminase (e.g., an APOBEC3A deaminase (A3A)) and an RNA-guided nickase and additional mRNAs encoding, for example, one or more UGIs and one or more gRNAs. Alternatively, the mRNA encoding the polypeptide comprising a cytidine deaminase (e.g., an APOBEC3A deaminase (A3A)) and an RNA-guided nickase, mRNA encoding one or more UGI, and mRNA encoding one or more gRNAs may be formulated in separate lipid nucleic acid assembly compositions. As such, one or multiple lipid nucleic acid assembly composition(s) may be delivered to a cell in vitro or in vivo.

In some embodiments, a method of modifying a target gene in a cell is provided, comprising delivering to the cell one or more lipid nucleic acid assembly compositions, optionally lipid nanoparticles, comprising:
(a) a first mRNA comprising a first open reading frame encoding a polypeptide comprising a cytidine deaminase (e.g., an APOBEC3A deaminase (A3A)) and an RNA-guided nickase;
(b) a second mRNA comprising a second open reading frame encoding a uracil glycosylase inhibitor (UGI); and
(c) one or more guide RNAs.

In some embodiments, parts (a) and (b) are in separate lipid nucleic acid assembly compositions. In some embodiments, parts (a) and (b) are in the same lipid nucleic acid assembly composition. In some embodiments, parts (a) and (c) are in separate lipid nucleic acid assembly compositions. In some embodiments, parts (a) and (c) are in the same lipid nucleic acid assembly composition. In some embodiments, parts (b) and (c) are in separate lipid nucleic acid assembly compositions. In some embodiments, parts (a) and (c) are in the same lipid nucleic acid assembly composition, and part (b) is in a separate lipid nucleic acid assembly composition. In some embodiments, parts (a), (b), and (c) are each in separate lipid nucleic acid assembly compositions. In some embodiments, parts (a), (b), and (c) are in the same lipid nucleic acid assembly composition. In some embodiments, the one or more guide RNAs are each in separate lipid nucleic acid assembly compositions.

In some embodiments, the method further comprise delivering one or more guide RNAs in one or more lipid nucleic acid assembly compositions that are separate from the lipid nucleic acid assembly compositions comprising the A3A and UGI.

In some embodiments, at least 2, 3, 4, 5, 6, 7, 8, 9, or 10 lipid nucleic acid assembly compositions are delivered to the cell. In some embodiments, the at least one lipid nucleic acid assembly composition comprises lipid nanoparticle (LNPs). In some embodiments, all lipid nucleic acid assembly compositions comprise LNPs. In some embodiments, at least one lipid nucleic acid assembly composition is a lipoplex composition.

In some embodiments, the lipid nucleic acid assembly composition, e.g., LNP composition, comprises an mRNA that encodes a polypeptide comprising a cytidine deaminase (e.g., an APOBEC3A deaminase (A3A)) and an RNA-guided nickase, as described herein. In some embodiments, the lipid nucleic acid assembly composition, e.g. LNP composition, comprises an mRNA that encodes a polypeptide comprising an APOBEC3A deaminase (A3A) and an RNA-guided nickase; and a gRNA.

In some embodiments, the lipid nucleic acid assembly composition comprises a first lipid nucleic acid assembly composition comprising an mRNA encoding a first polypeptide comprising a cytidine deaminase (e.g., an APOBEC3A deaminase (A3A)) and an RNA-guided nickase. In some embodiments, the lipid nucleic acid assembly composition further comprises a second lipid nucleic acid assembly composition comprising a gRNA.

In some embodiments, the lipid nucleic acid assembly composition comprises a first composition comprising a mRNA encoding a first polypeptide comprising a cytidine deaminase (e.g., an APOBEC3A deaminase (A3A)) and an RNA-guided nickase; and one or more second mRNA encoding a uracil glycosylase inhibitor (UGI). In some embodiments, the lipid nucleic acid assembly composition further comprises one or more gRNA.

In some embodiments, the lipid nucleic acid assembly composition further comprises a second lipid nucleic acid assembly composition comprising a gRNA. In some embodiments, the lipid nucleic acid assembly composition comprises first and second lipid nucleic acid assembly compositions, wherein the first composition comprises an mRNA encoding polypeptide comprising a cytidine deaminase (e.g., an APOBEC3A deaminase (A3A)) and an RNA-guided nickase; and the second composition comprises one or more mRNA encoding a uracil glycosylase inhibitor (UGI). In some embodiments, the first lipid nucleic acid assembly composition or the second lipid nucleic acid assembly composition further comprises one or more gRNA. In some embodiments, the lipid nucleic acid assembly composition further comprises a third lipid nucleic acid assembly composition comprising one or more gRNA.

In some embodiments, the lipid nucleic acid assembly composition comprises a first lipid nucleic acid assembly composition comprising an mRNA comprising an open reading frame encoding a first polypeptide comprising a cytidine deaminase (e.g., an APOBEC3A deaminase (A3A)) and an RNA-guided nickase and a second lipid nucleic acid assembly composition comprising one or more guide RNA (gRNA).

In some embodiments, the lipid nucleic acid assembly composition comprises a first composition comprising a first mRNA comprising a first open reading frame encoding a first polypeptide comprising a cytidine deaminase (e.g., an APOBEC3A deaminase (A3A)) and an RNA-guided nickase; and a second mRNA comprising one or more second open reading frame encoding a uracil glycosylase inhibitor (UGI). In some embodiments, the lipid nucleic acid assembly composition further comprises one or more gRNA. In some embodiments, the lipid nucleic acid assembly composition further comprises a second lipid nucleic acid assembly composition comprising one or more gRNA.

In some embodiments, the lipid nucleic acid assembly composition comprises first and second lipid nucleic acid assembly compositions, wherein the first composition comprises a first mRNA comprising a first open reading frame encoding a first polypeptide comprising a cytidine deaminase (e.g., an APOBEC3A deaminase (A3A)) and an RNA-guided nickase; the second composition comprises a second mRNA comprising a second open reading frame encoding a uracil glycosylase inhibitor (UGI). In some embodiments, the first lipid nucleic acid assembly composition or the second lipid nucleic acid assembly composition further comprises a gRNA. In some embodiments, the lipid nucleic acid assembly composition further comprises a third lipid nucleic acid assembly composition comprising a gRNA.

In some embodiments, the lipid nucleic acid assembly composition comprises a first composition comprising a first mRNA comprising a first open reading frame encoding a first polypeptide comprising a cytidine deaminase (e.g., an APOBEC3A deaminase (A3A)) and an RNA-guided nickase; and the second composition comprises a uracil glycosylase inhibitor (UGI). In some embodiments, the first lipid nucleic acid assembly composition or the second lipid nucleic acid assembly composition further comprises a gRNA. In some embodiments, the lipid nucleic acid assembly composition further comprises a third lipid nucleic acid assembly composition comprising a gRNA.

In some embodiments, the lipid nucleic acid assembly composition comprises first and second lipid nucleic acid assembly compositions, wherein the first composition comprises a polypeptide comprising a cytidine deaminase (e.g., an APOBEC3A deaminase (A3A)) and an RNA-guided nickase; the second composition comprises a second mRNA comprising a second open reading frame encoding a uracil glycosylase inhibitor (UGI). In some embodiments, the first lipid nucleic acid assembly composition or the second lipid nucleic acid assembly composition further comprises a gRNA. In some embodiments, the lipid nucleic acid assembly composition further comprises a third lipid nucleic acid assembly composition comprising a gRNA.

In certain embodiments, a lipid nucleic acid assembly composition may comprise mRNA, optionally a gRNA, an amine lipid, a helper lipid, a neutral lipid, and a stealth lipid. In certain lipid nucleic acid assembly compositions, the helper lipid is cholesterol. In other lipid nucleic acid assembly compositions, the neutral lipid is DSPC. In additional embodiments, the stealth lipid is PEG2k-DMG or PEG2k-C11. In certain embodiments, the lipid nucleic acid assembly composition comprises Lipid A or an equivalent of Lipid A; a helper lipid; a neutral lipid; a stealth lipid. In certain compositions, the amine lipid is Lipid A. In certain compositions, the amine lipid is Lipid A or an acetal analog thereof; the helper lipid is cholesterol; the neutral lipid is DSPC; and the stealth lipid is PEG2k-DMG.

Embodiments of the present disclosure also provide lipid nucleic acid assembly compositions described according to the molar ratio between the positively charged amine groups of the amine lipid (N) and the negatively charged phosphate groups (P) of the nucleic acid to be encapsulated. This may be mathematically represented by the equation N/P. In some embodiments, a lipid nucleic acid assembly composition may comprise a lipid component that comprises an amine lipid, a helper lipid, a neutral lipid, and a PEG lipid; and a nucleic acid component, wherein the N/P ratio is about 3 to 10. In some embodiments, a lipid nucleic acid assembly composition may comprise a lipid component that comprises an amine lipid, a helper lipid, and a PEG lipid; and a nucleic acid component, wherein the N/P ratio is about 3 to 10. In some embodiments, a lipid nucleic acid assembly composition may comprise a lipid component that comprises an amine lipid, a helper lipid, a neutral lipid, and a helper lipid; and an RNA component, wherein the N/P ratio is about 3 to 10. In some embodiments, a lipid nucleic acid assembly composition may comprise a lipid component that comprises an amine lipid, a helper lipid, and a PEG lipid; and an RNA component, such as an mRNA or gRNA, wherein the N/P ratio is about 3 to 10. In one embodiment, the N/P ratio may be about 5 to 7. In one embodiment, the N/P ratio may be about 3 to 7. In one embodiment, the N/P ratio may be about 4.5 to 8. In one embodiment, the N/P ratio may be about 6. In one embodiment, the N/P ratio may be 6±1. In one embodiment, the N/P ratio may be 6±0.5. In some embodiments, the N/P ratio will be ±30%, ±25%, ±20%, ±15%, ±10%, ±5%, or ±2.5% of the target N/P ratio. In certain embodiments, LNP inter-lot variability will be less than 15%, less than 10% or less than 5%.

In some embodiments, lipid nucleic acid assembly compositions are formed by mixing an aqueous RNA solution with an organic solvent-based lipid solution, e.g., 100% ethanol. Suitable solutions or solvents include or may contain: water, PBS, Tris buffer, NaCl, citrate buffer, ethanol, chloroform, diethylether, cyclohexane, tetrahydrofuran, methanol, isopropanol. A pharmaceutically acceptable buffer, e.g., for in vivo administration of lipid nucleic acid assembly compositions, may be used. In certain embodiments, a buffer is used to maintain the pH of the composition comprising lipid nucleic acid assembly compositions at or above pH 6.5. In certain embodiments, a buffer is used to maintain the pH of the composition comprising LNPs at or above pH 7.0. In certain embodiments, the composition has a pH ranging from about 7.2 to about 7.7. In additional embodiments, the composition has a pH ranging from about 7.3 to about 7.7 or ranging from about 7.4 to about 7.6. In further embodiments, the composition has a pH of about 7.2, 7.3, 7.4, 7.5, 7.6, or 7.7. The pH of a composition may be measured with a micro pH probe. In certain embodiments, a cryoprotectant is included in the composition. Non-limiting examples of cryoprotectants include sucrose, trehalose, glycerol, DMSO, and ethylene glycol. Exemplary compositions may include up to 10% cryoprotectant, such as, for example, sucrose. In certain embodiments, the lipid nucleic acid assembly composition may include about 1, 2, 3, 4, 5, 6, 7, 8, 9, or 10% cryoprotectant. In certain embodiments, the lipid nucleic acid assembly composition may include about 1, 2, 3, 4, 5, 6, 7, 8, 9, or 10% sucrose. In some embodiments, the lipid nucleic acid assembly composition may include a buffer. In some embodiments, the buffer may comprise a phosphate buffer (PBS), a Tris buffer, a citrate buffer, and mixtures thereof. In certain exemplary embodiments, the buffer comprises NaCl. In certain embodiments, NaCl is omitted. Exemplary amounts of NaCl may range from about 20 mM to about 45 mM. Exemplary amounts of NaCl may range from about 40 mM to about 50 mM. In some embodiments, the amount of NaCl is about 45 mM. In some embodiments, the buffer is a Tris buffer. Exemplary amounts of Tris may range from about 20 mM to about 60 mM. Exemplary amounts of Tris may range from about 40 mM to about 60 mM. In some embodiments, the amount of Tris is about 50 mM. In some embodiments, the buffer comprises NaCl and Tris. Certain exemplary embodiments of the lipid nucleic acid assembly compositions contain 5% sucrose and 45 mM NaCl in Tris buffer. In other exemplary embodiments, compositions contain sucrose in an amount of about 5% w/v, about 45 mM NaCl, and about 50 mM Tris at pH 7.5. The salt, buffer, and cryoprotectant amounts may be varied such that the osmolality of the overall formulation is maintained. For example, the final osmolality may be maintained at less than 450 mOsm/L. In further embodiments, the osmolality is between 350 and 250 mOsm/L. Certain embodiments have a final osmolality of 300+/−20 mOsm/L.

In some embodiments, microfluidic mixing, T-mixing, or cross-mixing is used. In certain aspects, flow rates, junction size, junction geometry, junction shape, tube diameter, solutions, and/or nucleic acids and lipid concentrations may be varied. Lipid nucleic acid assembly compositions may be concentrated or purified, e.g., via dialysis, tangential flow filtration, or chromatography. The lipid nucleic acid assembly compositions may be stored as a suspension, an emulsion, or a lyophilized powder, for example. In some embodiments, an lipid nucleic acid assembly composition is stored at 2-8° C., in certain aspects, the LNP compositions are stored at room temperature. In additional embodiments, a lipid nucleic acid assembly composition is stored frozen, for example at −20° C. or −80° C. In other embodiments, a lipid nucleic acid assembly composition is stored at a temperature ranging from about 0° C. to about −80° C. Frozen lipid nucleic acid assembly compositions may be thawed before use, for example on ice, at room temperature, or at 25° C.

The lipid nucleic acid assembly compositions may be, e.g., microspheres, a dispersed phase in an emulsion, micelles, or an internal phase in a suspension.

Moreover, in some embodiments, the lipid nucleic acid assembly compositions are biodegradable, in that they do not accumulate to cytotoxic levels in vivo at a therapeutically effective dose. In some embodiments, the lipid nucleic acid assembly compositions do not cause an innate immune response that leads to substantial adverse effects at a therapeutic dose level. In some embodiments, the lipid nucleic acid assembly compositions provided herein do not cause toxicity at a therapeutic dose level.

The LNPs disclosed herein may have a size (e.g., Z-average diameter) of about 1 to about 150 nm. In some embodiments, the LNPs have a size of about 10 to about 200 nm. In some embodiments, the LNPs have a size of about 50 to about 100 nm. In some embodiments, the LNPs have a size of about 60 to about 100 nm. In some embodiments, the LNPs have a size of about 75 to about 100 nm. In some embodiments, the LNP composition comprises a population of the LNP with an average diameter of about 20-100 nm. In some embodiments, the LNP composition comprises a population of the LNP with an average diameter of about 50-100 nm. In some embodiments, the LNP composition comprises a population of the LNP with an average diameter of about 60-100 nm. In some embodiments, the LNP composition comprises a population of the LNP with an average diameter of or about 75-100 nm. Unless indicated otherwise, all sizes referred to herein are the average sizes (diameters) of the fully formed nanoparticles, as measured by dynamic light scattering on a Malvern Zetasizer. The nanoparticle sample is diluted in phosphate buffered saline (PBS) so that the count rate is approximately 200-400 kcps. The data is presented as a weighted-average of the intensity measure (Z-average diameter).

In some embodiments, the LNPs are formed with an average encapsulation efficiency ranging from about 50% to about 100%. In some embodiments, the LNPs are formed with an average encapsulation efficiency ranging from about 50% to about 70%. In some embodiments, the LNPs are formed with an average encapsulation efficiency ranging from about 70% to about 90%. In some embodiments, the LNPs are formed with an average encapsulation efficiency ranging from about 90% to about 100%. In some embodiments, the LNPs are formed with an average encapsulation efficiency ranging from about 75% to about 95%.

In some embodiments, the LNPs are formed with an average molecular weight ranging from about 1.00E+05 g/mol to about 1.00E+10 g/mol. In some embodiments, the LNPs are formed with an average molecular weight ranging from about 5.00E+05 g/mol to about 7.00E+07 g/mol. In some embodiments, the LNPs are formed with an average molecular weight ranging from about 1.00E+06 g/mol to about 1.00E+10 g/mol. In some embodiments, the LNPs are formed with an average molecular weight ranging from about 1.00E+07 g/mol to about 1.00E+09 g/mol. In some embodiments, the LNPs are formed with an average molecular weight ranging from about 5.00E+06 g/mol to about 5.00E+09 g/mol.

In some embodiments, the polydispersity (Mw/Mn; the ratio of the weight averaged molar mass (Mw) to the number averaged molar mass (Mn)) may range from about 1.000 to about 2.000. In some embodiments, the Mw/Mn may range from about 1.00 to about 1.500. In some embodiments, the Mw/Mn may range from about 1.020 to about 1.400. In some embodiments, the Mw/Mn may range from about 1.010 to about 1.100. In some embodiments, the Mw/Mn may range from about 1.100 to about 1.350.

Dynamic Light Scattering (“DLS”) can be used to characterize the polydispersity index (“pdi”) and size of the LNPs of the present disclosure. DLS measures the scattering of light that results from subjecting a sample to a light source. PDI, as determined from DLS measurements, represents the distribution of particle size (around the mean particle size) in a population, with a perfectly uniform population having a PDI of zero. In some embodiments, the pdi may range from 0.005 to 0.75. In some embodiments, the pdi may range from 0.01 to 0.5. In some embodiments, the pdi may range from 0.02 to 0.4. In some embodiments, the pdi may range from 0.03 to 0.35. In some embodiments, the pdi may range from 0.1 to 0.35. In some embodiments, the pdi may range about zero to about 0.4, such as about zero to about 0.35. In some embodiments, the pdi may range from about zero to about 0.35, about zero to about 0.3, about zero to about 0.25, or about zero to about 0.2. In some embodiments, the pdi is less than about 0.08, 0.1, 0.15, 0.2, or 0.4.

In some embodiments, LNPs disclosed herein have a size of 1 to 250 nm. In some embodiments, the LNPs have a size of 10 to 200 nm. In further embodiments, the LNPs have a size of 20 to 150 nm. In some embodiments, the LNPs have a size of 50 to 150 nm. In some embodiments, the LNPs have a size of 50 to 100 nm. In some embodiments, the LNPs have a size of 50 to 120 nm. In some embodiments, the LNPs have a size of 75 to 150 nm. In some embodiments, the LNPs have a size of 30 to 200 nm. Unless indicated otherwise, all sizes referred to herein are the average sizes (diameters) of the fully formed nanoparticles, as measured by dynamic light scattering on a Malvern Zetasizer. The nanoparticle sample is diluted in phosphate buffered saline (PBS) so that the count rate is approximately 200-400 kcts. The data is presented as a weighted-average of the intensity measure. In some embodiments, the LNPs are formed with an average encapsulation efficiency ranging from 50% to 100%. In some embodiments, the LNPs are formed with an average encapsulation efficiency ranging from 50% to 70%. In some embodiments, the LNPs are formed with an average encapsulation efficiency ranging from 70% to 90%. In some embodiments, the LNPs are formed with an average encapsulation efficiency ranging from 90% to 100%. In some embodiments, the LNPs are formed with an average encapsulation efficiency ranging from 75% to 95%.

Electroporation is also a well-known means for delivery of cargo, and any electroporation methodology may be used for delivery of any one of the RNAs disclosed herein.

In some embodiments, the methods comprises a method for delivering a composition comprising the mRNA disclosed herein to an ex vivo cell, wherein the mRNA is encapsulated in an LNP. In some embodiments, the composition comprises the mRNA and one or more additional RNAs disclosed herein encapsulated in the LNP.

In some embodiments, a lipid nucleic acid assembly composition comprises a lipid component, wherein the lipid component comprises an amine lipid, a neutral lipid, a helper lipid, and a stealth lipid; and wherein the N/P ratio is about 1-10.

In some instances, the lipid component comprises Lipid A or its acetal analog, cholesterol, DSPC, and PEG-DMG; and wherein the N/P ratio is about 1-10. In some embodiments, the lipid component comprises: about 40-60 mol-% amine lipid; about 5-15 mol-% neutral lipid; and about 1.5-10 mol-% PEG lipid, wherein the remainder of the lipid component is helper lipid, and wherein the N/P ratio of the lipid nucleic acid assembly composition is about 3-10. In some embodiments, the lipid component comprises about 50-60 mol-% amine lipid; about 8-10 mol-% neutral lipid; and about 2.5-4 mol-% PEG lipid, wherein the remainder of the lipid component is helper lipid, and wherein the N/P ratio of the lipid nucleic acid assembly composition is about 3-8. In some instances, the lipid component comprises: about 50-60 mol-% amine lipid; about 5-15 mol-% DSPC; and about 2.5-4 mol-% PEG lipid, wherein the remainder of the lipid component is cholesterol, and wherein the N/P ratio of the lipid nucleic acid assembly composition is about 3-8. In some instances, the lipid component comprises: 48-53 mol-% Lipid A; about 8-10 mol-% DSPC; and 1.5-10 mol-% PEG lipid, wherein the remainder of the lipid component is cholesterol, and wherein the N/P ratio of the lipid nucleic acid assembly composition is 3-8±0.2.

In some embodiments, the lipid component comprises about 50-60 mol-% amine lipid such as Lipid A, about 8-10 mol-% neutral lipid; and about 2.5-4 mol-% stealth lipid (e.g., a PEG lipid), wherein the remainder of the lipid component is helper lipid, and wherein the N/P ratio of the lipid nucleic acid assembly composition is about 6. In some embodiments, the lipid component comprises about 50-60 mol-% amine lipid such as Lipid A; about 27-39.5 mol-% helper lipid; about 8-10 mol-% neutral lipid; and about 2.5-4 mol-% stealth lipid (e.g., a PEG lipid), wherein the N/P ratio of the lipid nucleic acid assembly composition is about 5-7 (e.g., about 6). In some embodiments, the lipid component comprises about 50-60 mol-% amine lipid such as Lipid A; about 5-15 mol-% neutral lipid; and about 2.5-4 mol-% Stealth lipid (e.g., a PEG lipid), wherein the remainder of the lipid component is helper lipid, and wherein the N/P ratio of the lipid nucleic acid assembly composition is about 3-10. In some embodiments, the lipid component comprises about 40-60 mol-% amine lipid such as Lipid A; about 5-15 mol-% neutral lipid; and about 2.5-4 mol-% Stealth lipid (e.g., a PEG lipid), wherein the remainder of the lipid component is helper lipid, and wherein the N/P ratio of the lipid nucleic acid assembly composition is about 6. In some embodiments, the lipid component comprises about 50-60 mol-% amine lipid such as Lipid A; about 5-15 mol-% neutral lipid; and about 1.5-10 mol-% Stealth lipid (e.g., a PEG lipid), wherein the remainder of the lipid component is helper lipid, and wherein the N/P ratio of the lipid nucleic acid assembly composition is about 6. In some embodiments, the lipid component comprises about 40-60 mol-% amine lipid such as Lipid A; about 0-10 mol-% neutral lipid; and about 1.5-10 mol-% Stealth lipid (e.g., a PEG lipid), wherein the remainder of the lipid component is helper lipid, and wherein the N/P ratio of the lipid nucleic acid assembly composition is about 3-10. In some embodiments, the lipid component comprises about 40-60 mol-% amine lipid such as Lipid A; less than about 1 mol-% neutral lipid; and about 1.5-10 mol-% Stealth lipid (e.g., a PEG lipid), wherein the remainder of the lipid component is helper lipid, and wherein the N/P ratio of the lipid nucleic acid assembly composition is about 3-10. In some embodiments, the lipid component comprises about 40-60 mol-% amine lipid such as Lipid A; and about 1.5-10 mol-% Stealth lipid (e.g., a PEG lipid), wherein the remainder of the lipid component is helper lipid, wherein the N/P ratio of the lipid nucleic acid assembly composition is about 3-10, and wherein the lipid nucleic acid assembly composition is essentially free of or free of neutral phospholipid. In some embodiments, the lipid component comprises about 50-60 mol-% amine lipid such as Lipid A; about 8-10 mol-% neutral lipid; and about 2.5-4 mol-% Stealth lipid (e.g., a PEG lipid), wherein the remainder of the lipid component is helper lipid, and wherein the N/P ratio of the Lipid nucleic acid assembly composition is about 3-7.

In some embodiments, the amine lipid is present at about 50 mol-%. In some embodiments, the neutral lipid is present at about 9 mol-%. In some embodiments, the stealth lipid is present at about 3 mol-%. In some embodiments, the helper lipid is present at about 38 mol-%.

In some embodiments, the lipid component comprises, consists essentially of, or consists of: about 50 mol-% amine lipid such as Lipid A; about 9 mol-% neutral lipid such as DSPC; about 3 mol-% of a stealth lipid such as a PEG lipid, such as PEG2k-DMG, and the remainder of the lipid component is helper lipid such as cholesterol, wherein the N/P ratio of the lipid nucleic acid assembly composition is about 6. In some embodiments, the amine lipid is Lipid A. In some embodiments, the neutral lipid is DSPC. In some embodiments, the stealth lipid is a PEG lipid. In some embodiments, the stealth lipid is a PEG2k-DMG. In some embodiments, the helper lipid is cholesterol. In some embodiments, the lipid comprises a lipid component and the lipid component comprises: about 50 mol-% Lipid A; about 9 mol-% DSPC; about 3 mol-% of PEG2k-DMG, and the remainder of the lipid component is cholesterol wherein the N/P ratio of the lipid nucleic acid assembly composition is about 6.

In some embodiments, the lipid component comprises, consists essentially of, or consists of: about 25 to 45 mol-% amine lipid such as Lipid A; about 10 to 30 mol-% neutral lipid such as DSPC; about 1.5 to 3.5 mol-% of a stealth lipid such as a PEG lipid, such as PEG2k-DMG, and about 25 to 65 mol % helper lipid such as cholesterol, wherein the N/P ratio of the lipid nucleic acid assembly composition is about 6. In some embodiments, the lipid component comprises, consists essentially of, or consists of: about 35 mol-% amine lipid such as Lipid A; about 15 mol-% neutral lipid such as DSPC; about 2.5 mol-% of a stealth lipid such as a PEG lipid, such as PEG2k-DMG, and the remainder of the lipid component is helper lipid such as cholesterol, wherein the N/P ratio of the lipid nucleic acid assembly composition is about 6. In some embodiments, the amine lipid is Lipid A. In some embodiments, the neutral lipid is DSPC. In some embodiments, the stealth lipid is a PEG lipid. In some embodiments, the stealth lipid is a PEG2k-DMG. In some embodiments, the helper lipid is cholesterol. In some embodiments, the lipid comprises a lipid component and the lipid component comprises: about 35 mol-% Lipid A; about 15 mol-% DSPC; about 2.5 mol-% of PEG2k-DMG, and the remainder of the lipid component is cholesterol wherein the N/P ratio of the lipid nucleic acid assembly composition is about 6.

### I. Exemplary Uses, Methods, and Treatments

In some embodiments, a nucleic acid (e.g., mRNA), polypeptide, composition, or lipid nucleic acid assembly composition disclosed herein is for use in genome editing, e.g., editing a target gene, or modifying a target gene. In some embodiments, a nucleic acid (e.g., mRNA), polypeptide, composition, or lipid nucleic acid assembly composition disclosed herein is for use in modifying a target gene, e.g., altering its sequence or epigenetic status. In some embodiments, the nucleic acid (e.g., mRNA), polypeptide, composition, or lipid nucleic acid assembly composition disclosed herein is for use in the manufacture of a medicament for genome editing or modifying a target gene.

In some embodiments, the use of a nucleic acid (e.g., mRNA), polypeptide, composition, or lipid nucleic acid assembly composition disclosed herein is provided for the preparation of a medicament for genome editing, e.g., editing a target gene. In some embodiments, the use of a nucleic acid (e.g., mRNA), polypeptide, composition, or lipid nucleic acid assembly composition is provided for the preparation of a medicament for modifying a target gene, e.g., altering its sequence or epigenetic status. In some embodiments, the use of a nucleic acid (e.g., mRNA), polypeptide, composition, or lipid nucleic acid assembly composition disclosed herein is provided for the preparation of a medicament for causing C-to-T conversion within a target gene.

In some embodiments, a method of genome editing or modifying a target gene is provided, the method comprising delivering to a cell the mRNA, composition, or lipid nanoparticle(s) described herein.

In some embodiments, the method generates a cytosine (C) to thymine (T) conversion within a target gene.

In some embodiments, the method causes at least 50% C-to-T conversion relative to the total edits in the target sequence. As used herein, the “total edits in the target sequence” is the sum of each read with an indel or at least one conversion, wherein an indel can comprise more than one nucleotide. Indel is calculated as the total number of sequencing reads with one or more base inserted or deleted within the 20 bp scoring region divided by the total number of sequencing reads, including wild type. C-to-T conversions or C-to-A/G conversions were scored in a 40 bp region including 10 bp upstream and 10 bp downstream of the 20 bp sgRNA target sequence. Any sequencing methods (e.g., NGS) that allow reading of sequences diverged from the wild-type alignment may be used. In some embodiments, the method causes at least 50%, at least 55%, at least 60%, at least 65%, at least 70%, at least 75%, at least 80%, at least 85%, at least 90%, at least 95%, at least 96%, at least 97%, at least 98%, or at least 99% C-to-T conversion relative to the total edits in the target sequence.

In some embodiments, the ratio of C-to-T conversion to unintended edits is larger than 1:1. As used herein, an “unintended edit” is any edit in the target region that is not a C-to-T conversion. In some embodiments, the ratio of C-to-T conversion to unintended edits is larger than 2:1, larger than 3:1, larger than 4:1, larger than 5:1, larger than 6:1, larger than 7:1, or larger than 8:1. In some embodiments, the ratio of C-to-T conversion to unintended edits is from 2:1 to 99:1. In some embodiments, the ratio of C-to-T conversion to unintended edits is 2:1, 3:1, 4:1, 5:1, 6:1, 7:1, or 8:1.

In some embodiments, the method causes the A3A to make a base edit corresponding to any one of positions −1 to 10 relative to the 5′ end of the guide sequence.

In some embodiments, the method causes the A3A to make a base edit at a position 1, 2, 3, 4, 5, 6, 7, 8, 9, or 10 nucleotides from the 5′ end of the guide sequence.

In some embodiments, the nickase is a SpyCas9 nickase, and the method causes the cytidine deaminase to make a base edit at a cytidine present at position 1, 2, 3, 4, 5, 6, 7, 8, 9, 10, or 11 nucleotides from the 5′ end of the guide sequence.

In some embodiments, the nickase is a NmeCas9 nickase, and the method causes the cytidine deaminase to make a base edit at a cytidine present at position 4, 5, 6, 7, 8, 9, 10, 11, 12, 13, 14, 15, 16, 17, 18, 19, or 20 nucleotides from the 5′ end of the guide sequence.

In some embodiments, the composition comprises a first mRNA comprising a first open reading frame encoding a polypeptide comprising an A3A and an RNA-guided nickase, and a second mRNA comprising a second open reading frame encoding a uracil glycosylase inhibitor (UGI), and a gRNA; and the first mRNA, the second mRNA, and the gRNA if present, delivered at a ratio of about 6:2:3 (w:w:w). In some embodiments, the target gene is in a subject, such as a mammal, such as a human.

In some embodiments, methods are provided for modifying a target gene comprising delivering to a cell a first mRNA comprising a first open reading frame encoding a first polypeptide comprising a cytidine deaminase (e.g., an APOBEC3A deaminase (A3A)) and an RNA-guided nickase, a second mRNA comprising a second open reading frame encoding a uracil glycosylase inhibitor (UGI), wherein the second mRNA is different from the first mRNA, and at least one guide RNA (gRNA).

In some embodiments, methods are provided for modifying a target gene in a cell, comprising delivering to the cell one or more lipid nucleic acid assembly compositions, optionally lipid nanoparticles, comprising: (a) a first mRNA comprising a first open reading frame encoding a polypeptide comprising a cytidine deaminase (e.g., an APOBEC3A deaminase (A3A)) and an RNA-guided nickase; (b) a second mRNA comprising a second open reading frame encoding a uracil glycosylase inhibitor (UGI); and (c) one or more guide RNAs. In some embodiments, the one or more guide RNAs are each in separate lipid nucleic acid assembly compositions.

In some embodiments, methods are provided for modifying a target gene in a cell, comprising delivering to the cell one or more lipid nucleic acid assembly compositions, optionally lipid nanoparticles, comprising: (a) a first mRNA comprising a first open reading frame encoding a polypeptide comprising a cytidine deaminase (e.g., an APOBEC3A deaminase (A3A)) and an RNA-guided nickase; (b) a second mRNA comprising a second open reading frame encoding a uracil glycosylase inhibitor (UGI); and (c) one or more guide RNAs, wherein the method comprises one gRNA that targets a gene that reduces or eliminates MHC class I expression on the surface of a cell, and/or one gRNA that targets a gene that reduces or eliminates MHC class II expression on the surface of a cell, and/or one gRNA that targets a gene that reduces or eliminates endogenous TCR expression.

In some embodiments, methods are provided for modifying a target gene in a cell, comprising delivering to the cell one or more lipid nucleic acid assembly compositions, optionally lipid nanoparticles, comprising: (a) a first mRNA comprising a first open reading frame encoding a polypeptide comprising a cytidine deaminase (e.g., an APOBEC3A deaminase (A3A)) and an RNA-guided nickase; (b) a second mRNA comprising a second open reading frame encoding a uracil glycosylase inhibitor (UGI); and (c) one or more guide RNAs, wherein the method comprises at least two gRNAs selected from: one gRNA that targets a gene that reduces or eliminates MHC class I expression on the surface of a cell, one gRNA that targets a gene that reduces or eliminates MHC class II expression on the surface of a cell, and one gRNA that targets a gene that reduces or eliminates endogenous TCR expression.

In some embodiments, methods are provided for modifying a target gene in a cell, comprising delivering to the cell one or more lipid nucleic acid assembly compositions, optionally lipid nanoparticles, comprising: (a) a first mRNA comprising a first open reading frame encoding a polypeptide comprising a cytidine deaminase (e.g., an APOBEC3A deaminase (A3A)) and an RNA-guided nickase; (b) a second mRNA comprising a second open reading frame encoding a uracil glycosylase inhibitor (UGI); and (c) one or more guide RNAs, wherein the method comprises one gRNA that targets a gene that reduces or eliminates MHC class I expression on the surface of a cell, one gRNA that targets a gene that reduces or eliminates MHC class II expression on the surface of a cell, and one gRNA that targets a gene that reduces or eliminates endogenous TCR expression.

In some embodiments, methods are provided for modifying a target gene in a cell, comprising delivering to the cell one or more lipid nucleic acid assembly compositions, optionally lipid nanoparticles, comprising: (a) a first mRNA comprising a first open reading frame encoding a polypeptide comprising a cytidine deaminase (e.g., an APOBEC3A deaminase (A3A)) and an RNA-guided nickase; (b) a second mRNA comprising a second open reading frame encoding a uracil glycosylase inhibitor (UGI); and (c) one or more guide RNAs, wherein the method comprises one gRNA that targets a gene that reduces or eliminates expression of HLA-A on the surface of a cell, and/or one gRNA that targets a gene that reduces or eliminates MHC class II expression on the surface of a cell, and/or one gRNA that targets a gene that reduces or eliminates endogenous TCR expression.

In some embodiments, methods are provided for modifying a target gene in a cell, comprising delivering to the cell one or more lipid nucleic acid assembly compositions, optionally lipid nanoparticles, comprising: (a) a first mRNA comprising a first open reading frame encoding a polypeptide comprising a cytidine deaminase (e.g., an APOBEC3A deaminase (A3A)) and an RNA-guided nickase; (b) a second mRNA comprising a second open reading frame encoding a uracil glycosylase inhibitor (UGI); and (c) one or more guide RNAs, wherein the method comprises at least two gRNAs selected from: one gRNA that targets a gene that reduces or eliminates expression of HLA-A on the surface of a cell, one gRNA that targets a gene that reduces or eliminates MHC class II expression on the surface of a cell, and one gRNA that targets a gene that reduces or eliminates endogenous TCR expression.

In some embodiments, methods are provided for modifying a target gene in a cell, comprising delivering to the cell one or more lipid nucleic acid assembly compositions, optionally lipid nanoparticles, comprising: (a) a first mRNA comprising a first open reading frame encoding a polypeptide comprising a cytidine deaminase (e.g., an APOBEC3A deaminase (A3A)) and an RNA-guided nickase; (b) a second mRNA comprising a second open reading frame encoding a uracil glycosylase inhibitor (UGI); and (c) one or more guide RNAs, wherein the method comprises one gRNA that targets a gene that reduces or eliminates expression of HLA-A on the surface of a cell, one gRNA that targets a gene that reduces or eliminates MHC class II expression on the surface of a cell, and one gRNA that targets a gene that reduces or eliminates endogenous TCR expression.

In some embodiments, methods are provided for modifying a target gene in a cell, comprising delivering to the cell one or more lipid nucleic acid assembly compositions, optionally lipid nanoparticles, comprising: (a) a first mRNA comprising a first open reading frame encoding a polypeptide comprising a cytidine deaminase (e.g., an APOBEC3A deaminase (A3A)) and an RNA-guided nickase; (b) a second mRNA comprising a second open reading frame encoding a uracil glycosylase inhibitor (UGI); and (c) one or more guide RNAs, wherein the method comprises one gRNA selected from a gRNA that targets TRAC, TRBC, B2M, HLA-A, or CIITA. In some embodiments, one gRNA targets TRAC. In some embodiments, one gRNA targets TRBC. In some embodiments, one gRNA targets B2M. In some embodiments, one gRNA targets HLA-A. In some embodiments, one gRNA targets CIITA.

In some embodiments, methods are provided for modifying a target gene in a cell, comprising delivering to the cell one or more lipid nucleic acid assembly compositions, optionally lipid nanoparticles, comprising: (a) a first mRNA comprising a first open reading frame encoding a polypeptide comprising a cytidine deaminase (e.g., an APOBEC3A deaminase (A3A)) and an RNA-guided nickase; (b) a second mRNA comprising a second open reading frame encoding a uracil glycosylase inhibitor (UGI); and (c) one or more guide RNAs, wherein the method comprises at least two gRNAs selected from a gRNA that targets TRAC, TRBC, or B2M, wherein the two guide RNAs do not target the same gene. In some embodiments, the gRNAs are each in separate lipid nucleic acid assembly compositions.

In some embodiments, methods are provided for modifying a target gene in a cell, comprising delivering to the cell one or more lipid nucleic acid assembly compositions, optionally lipid nanoparticles, comprising: (a) a first mRNA comprising a first open reading frame encoding a polypeptide comprising a cytidine deaminase (e.g., an APOBEC3A deaminase (A3A)) and an RNA-guided nickase; (b) a second mRNA comprising a second open reading frame encoding a uracil glycosylase inhibitor (UGI); and (c) one or more guide RNAs, wherein the method comprises at least two gRNAs selected from a gRNA that targets TRAC, TRBC, or HLA-A wherein the two guide RNAs do not target the same gene. In some embodiments, the gRNAs are each in separate lipid nucleic acid assembly compositions.

In some embodiments, methods are provided for modifying a target gene in a cell, comprising delivering to the cell one or more lipid nucleic acid assembly compositions, optionally lipid nanoparticles, comprising: (a) a first mRNA comprising a first open reading frame encoding a polypeptide comprising a cytidine deaminase (e.g., an APOBEC3A deaminase (A3A)) and an RNA-guided nickase; (b) a second mRNA comprising a second open reading frame encoding a uracil glycosylase inhibitor (UGI); and (c) one or more guide RNAs, wherein the method comprises at least two gRNAs selected from a gRNA that targets TRAC, TRBC, HLA-A, wherein the two guide RNAs do not target the same gene. In some embodiments, the gRNAs are each in separate lipid nucleic acid assembly compositions.

In some embodiments, methods are provided for modifying a target gene in a cell, comprising delivering to the cell one or more lipid nucleic acid assembly compositions, optionally lipid nanoparticles, comprising: (a) a first mRNA comprising a first open reading frame encoding a polypeptide comprising a cytidine deaminase (e.g., an APOBEC3A deaminase (A3A)) and an RNA-guided nickase; (b) a second mRNA comprising a second open reading frame encoding a uracil glycosylase inhibitor (UGI); and (c) one or more guide RNAs, wherein the method comprises one guide RNA that targets TRAC, and one gRNA that targets TRBC. In some embodiments, the gRNAs are each in separate lipid nucleic acid assembly compositions.

In some embodiments, methods are provided for modifying a target gene in a cell, comprising delivering to the cell one or more lipid nucleic acid assembly compositions, optionally lipid nanoparticles, comprising: (a) a first mRNA comprising a first open reading frame encoding a polypeptide comprising a cytidine deaminase (e.g., an APOBEC3A deaminase (A3A)) and an RNA-guided nickase; (b) a second mRNA comprising a second open reading frame encoding a uracil glycosylase inhibitor (UGI); and (c) one or more guide RNAs, wherein the method comprises one guide RNA that targets B2M, and one gRNA that targets CIITA. In some embodiments, the gRNAs are each in separate lipid nucleic acid assembly compositions.

In some embodiments, methods are provided for modifying a target gene in a cell, comprising delivering to the cell one or more lipid nucleic acid assembly compositions, optionally lipid nanoparticles, comprising: (a) a first mRNA comprising a first open reading frame encoding a polypeptide comprising a cytidine deaminase (e.g., an APOBEC3A deaminase (A3A)) and an RNA-guided nickase; (b) a second mRNA comprising a second open reading frame encoding a uracil glycosylase inhibitor (UGI); and (c) one or more guide RNAs, wherein the method comprises one guide RNA that targets HLA-A, and one gRNA that targets CIITA. In some embodiments, the gRNAs are each in separate lipid nucleic acid assembly compositions. In some embodiments, the cell is homozygous for HLA-B and homozygous for HLA-C.

In some embodiments, methods are provided for modifying a target gene in a cell, comprising delivering to the cell one or more lipid nucleic acid assembly compositions, optionally lipid nanoparticles, comprising: (a) a first mRNA comprising a first open reading frame encoding a polypeptide comprising a cytidine deaminase (e.g., an APOBEC3A deaminase (A3A)) and an RNA-guided nickase; (b) a second mRNA comprising a second open reading frame encoding a uracil glycosylase inhibitor (UGI); and (c) one or more guide RNAs, wherein the method comprises one guide RNA that targets TRAC, and one gRNA that targets TRBC, and one gRNA that targets B2M. In some embodiments, the gRNAs are each in separate lipid nucleic acid assembly compositions.

In some embodiments, methods are provided for modifying a target gene in a cell, comprising delivering to the cell one or more lipid nucleic acid assembly compositions, optionally lipid nanoparticles, comprising: (a) a first mRNA comprising a first open reading frame encoding a polypeptide comprising a cytidine deaminase (e.g., an APOBEC3A deaminase (A3A)) and an RNA-guided nickase; (b) a second mRNA comprising a second open reading frame encoding a uracil glycosylase inhibitor (UGI); and (c) one or more guide RNAs, wherein the method comprises one guide RNA that targets TRAC, and one gRNA that targets TRBC, and one gRNA that targets HLA-A. In some embodiments, the gRNAs are each in separate lipid nucleic acid assembly compositions. In some embodiments, the cell is homozygous for HLA-B and homozygous for HLA-C.

In some embodiments, methods are provided for modifying a target gene in a cell, comprising delivering to the cell one or more lipid nucleic acid assembly compositions, optionally lipid nanoparticles, comprising: (a) a first mRNA comprising a first open reading frame encoding a polypeptide comprising a cytidine deaminase (e.g., an APOBEC3A deaminase (A3A)) and an RNA-guided nickase; (b) a second mRNA comprising a second open reading frame encoding a uracil glycosylase inhibitor (UGI); and (c) one or more guide RNAs, wherein the method comprises one guide RNA that targets TRAC, and one gRNA that targets TRBC, one gRNA that targets B2M, and one gRNA that targets CIITA. In some embodiments, the gRNAs are each in separate lipid nucleic acid assembly compositions.

In some embodiments, methods are provided for modifying a target gene in a cell, comprising delivering to the cell one or more lipid nucleic acid assembly compositions, optionally lipid nanoparticles, comprising: (a) a first mRNA comprising a first open reading frame encoding a polypeptide comprising a cytidine deaminase (e.g., an APOBEC3A deaminase (A3A)) and an RNA-guided nickase; (b) a second mRNA comprising a second open reading frame encoding a uracil glycosylase inhibitor (UGI); and (c) one or more guide RNAs, wherein the method comprises one guide RNA that targets TRAC, and one gRNA that targets TRBC, one gRNA that targets HLA-A, and one gRNA that targets CIITA. In some embodiments, the gRNAs are each in separate lipid nucleic acid assembly compositions. In some embodiments, the cell is homozygous for HLA-B and homozygous for HLA-C.

In some embodiments, a cell is provided comprising a composition comprising a first mRNA comprising a first open reading frame encoding a polypeptide comprising a cytidine deaminase (e.g., an APOBEC3A deaminase (A3A)) and an RNA-guided nickase, and a second mRNA comprising a second open reading frame encoding a uracil glycosylase inhibitor (UGI), wherein the second mRNA is different from the first mRNA.

In some embodiments, an engineered cell is provided comprising at least one base edit and/or indel, wherein the base edit and/or indel is made by contacting a cell with a composition comprising a first mRNA comprising a first open reading frame encoding a polypeptide comprising a cytidine deaminase (e.g., an APOBEC3A deaminase (A3A)) and an RNA-guided nickase, and a second mRNA comprising a second open reading frame encoding a uracil glycosylase inhibitor (UGI), wherein the second mRNA is different from the first mRNA.

In some embodiments the cell is a human cell. In some embodiments the genetically modified cell is referred to as an engineered cell. An engineered cell refers to a cell (or progeny of a cell) comprising an engineered genetic modification, e.g. that has been contacted with a gene editing system and genetically modified by the gene editing system. The terms “engineered cell” and “genetically modified cell” are used interchangeably throughout. The engineered cell may be any of the exemplary cell types disclosed herein. In some embodiments, the cell is an allogeneic cell.

In some embodiments, the cell is an immune cell. As used herein, “immune cell” refers to a cell of the immune system, including e.g., a lymphocyte (e.g., T cell, B cell, natural killer cell (“NK cell”, and NKT cell, or iNKT cell)), monocyte, macrophage, mast cell, dendritic cell, or granulocyte (e.g., neutrophil, eosinophil, and basophil). In some embodiments, the cell is a primary immune cell. In some embodiments, the immune system cell may be selected from CD3+, CD4+ and CD8+ T cells, regulatory T cells (Tregs), B cells, NK cells, and dendritic cells (DC). In some embodiments, the immune cell is allogeneic. In some embodiments, the cell is a lymphocyte. In some embodiments, the cell is an adaptive immune cell. In some embodiments, the cell is a T cell. In some embodiments, the cell is a B cell. In some embodiments, the cell is a NK cell. In some embodiments, the lymphocyte is allogeneic.

In some embodiments, the genome editing or modification of the target gene is in vivo. In some embodiments, the genome editing or modification of the target gene is in an isolated or cultured cell.

In some embodiments, the target gene is in an organ, such as a liver, such as a mammalian liver, such as a human liver. In some embodiments, the target gene is in a liver cell, such as a mammalian liver cell, such as a human liver cell. In some embodiments, the target gene is in a hepatocyte, such as a mammalian hepatocyte, such as a human hepatocyte. In some embodiments, the liver cell or hepatocyte is in situ. In some embodiments, the liver cell or hepatocyte is isolated, e.g., in a culture, such as in a primary culture.

In some embodiments, the genome editing or modification of the target gene inactivates a splice donor or splice acceptor site.

Also provided are methods corresponding to the uses disclosed herein, which comprise administering the nucleic acid (e.g., mRNA), polypeptide, composition, or lipid nucleic acid assembly composition disclosed herein to a subject or contacting a cell such as those described above with the nucleic acid (e.g., mRNA), polypeptide, composition, or lipid nucleic acid assembly composition disclosed herein.

In some embodiments the nucleic acid (e.g., mRNA), polypeptide, composition, or lipid nucleic acid assembly composition disclosed herein is administered intravenously for any of the uses discussed above concerning organisms, organs, or cells in situ.

In any of the foregoing embodiments involving a subject, the subject can be mammalian. In any of the foregoing embodiments involving a subject, the subject can be human. In any of the foregoing embodiments involving a subject, the subject can be a cow, pig, monkey, sheep, dog, cat, fish, or poultry.

In some embodiments, the nucleic acid (e.g., mRNA), polypeptide, composition, or lipid nucleic acid assembly composition disclosed herein is administered intravenously or for intravenous administration.

In some embodiments, the genome editing or modification of the target gene knocks down expression of the target gene. In some embodiments, the genome editing or modification of the target gene knocks down expression of the target gene by at least 50%, 55%, 60%, 65%, 70%, 75%, or 80%. In some embodiments, the genome editing or modification of the target gene produces a missense mutation in the gene.

In some embodiments, a single administration of a nucleic acid (e.g., mRNA), polypeptide, composition, or lipid nucleic acid assembly composition disclosed herein is sufficient to knock down expression of the target gene product. In some embodiments, a single administration of a nucleic acid (e.g., mRNA), polypeptide, composition, or lipid nucleic acid assembly composition disclosed herein is sufficient to knock out expression of the target gene product. In other embodiments, more than one administration of a nucleic acid (e.g., mRNA), polypeptide, composition, or lipid nucleic acid assembly composition disclosed herein may be beneficial to maximize editing via cumulative effects.

In some embodiments, the efficacy of treatment with a nucleic acid (e.g., mRNA), polypeptide, composition, or lipid nucleic acid assembly composition disclosed herein is seen at 1 year, 2 years, 3 years, 4 years, 5 years, or 10 years after delivery.

In some embodiments, treatment slows or halts disease progression.

In some embodiments, treatment results in improvement, stabilization, or slowing of change in organ function or symptoms of disease of an organ.

In some embodiments, efficacy of treatment is measured by increased survival time of the subject.

1. Exemplary Guide RNAs, Compositions, Methods, and Engineered Cells for TRAC and TRBC Editing

The disclosure provides a guide RNA that target TRAC. Guide sequences targeting the TRAC gene are shown in Table 5A at SEQ ID NOs: 706-721.

The disclosure provides a guide RNA that target TRBC. Guide sequences targeting the TRBC gene are shown in Table 5B at SEQ ID NOs: 618-669.

In some embodiments, the guide sequences are complementary to the corresponding genomic region shown in the tables below, according to coordinates from human reference genome hg38. Guide sequences of further embodiments may be complementary to sequences in the close vicinity of the genomic coordinate listed in any of Tables 5A and 5B. For example, guide sequences of further embodiments may be complementary to sequences that comprise 15 consecutive nucleotides±10 nucleotides of a genomic coordinate listed in any of Tables 5A an 5B.

As described in the preceding sections, each of the guide sequences shown in Table 5A and Table 5B may further comprise additional nucleotides to form a crRNA, e.g., with the following exemplary nucleotide sequence following the guide sequence at its 3′ end: GUUUUAGAGCUAUGCUGUUUUG (SEQ ID NO: 139) in 5′ to 3′ orientation. In the case of a sgRNA, the guide sequences may further comprise additional nucleotides to form a sgRNA, e.g., with the following exemplary nucleotide sequence following the 3′ end of the guide sequence: GUUUUAGAGCUAGAAAUAGCAAGUUAAAAUAAGGCUAGUCCGUUAUCAACUU GAAAAAGUGGCACCGAGUCGGUGCUUUU (SEQ ID NO: 140) in 5′ to 3′ orientation. The guide sequences may further comprise additional nucleotides to form a sgRNA, e.g.,

In some embodiments, the sgRNA comprises the modification pattern shown below in SEQ ID NO: 141, where N is any natural or non-natural nucleotide, and where the totality of the N's comprise a guide sequence as described herein and the modified sgRNA comprises the following sequence: mN*mN*mN*NNNNNNNNNNNNNNNNNGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 141), where “N” may be any natural or non-natural nucleotide. For example, encompassed herein is SEQ ID NO: 141, where the N's are replaced with any of the guide sequences disclosed herein. The modifications remain as shown in SEQ ID NO: 141 despite the substitution of N's for the nucleotides of a guide. That is, although the nucleotides of the guide replace the “N's”, the first three nucleotides are 2′OMe modified and there are phosphorothioate linkages between the first and second nucleotides, the second and third nucleotides and the third and fourth nucleotides.

In some embodiments, the gRNA targeting TRAC comprises a guide sequence chosen from: i) SEQ ID NOs: 706-721; ii) at least 17, 18, 19, or 20 contiguous nucleotides of a sequence selected from SEQ ID NOs: 706-721; iii) a guide sequence at least 95%, 90%, or 85% identical to a sequence selected from SEQ ID NOs: 706-721; iv) a sequence that comprises 10 contiguous nucleotides±10 nucleotides of a genomic coordinate listed in Table 5A; v) at least 17, 18, 19, or 20 contiguous nucleotides of a sequence from (iv); or vi) a guide sequence that is at least 95%, 90%, or 85% identical to a sequence selected from (v).

TABLE 5A
TRAC guide sequences, guide RNA sequences, and chromosomal
coordinates

| | SEQ ID | | | | |
|---|---|---|---|---|---|
| | NO to the | | Exemplary Full | | Genomic |
| Guide | Guide | Guide | Sequence (SEQ ID | Exemplary Mod Sequence | Coordinates |
| ID | Sequence | Sequence | NOS: 747-762) | (SEQ ID NOS: 853-868) | (hg38) |
| G016019 | 706 | AACAAA | AACAAAUGUGU | mA*mA*mC*AAAUGUGUC | chr14: |
| | | UGUGUC | CACAAAGUAGU | ACAAAGUAGUUUUAGAm | 22547596- |
| | | ACAAAG | UUUAGAGCUAG | GmCmUmAmGmAmAmAm | 22547616 |
| | | UA | AAAUAGCAAGU | UmAmGmCAAGUUAAAAU | |
| | | | UAAAAUAAGGC | AAGGCUAGUCCGUUAUC | |
| | | | UAGUCCGUUAU | AmAmCmUmUmGmAmAm | |
| | | | CAACUUGAAAA | AmAmAmGmUmGmGmCm | |
| | | | AGUGGCACCGA | AmCmCmGmAmGmUmCm | |
| | | | GUCGGUGCUUU | GmGmUmGmCmU*mU*mU | |
| | | | U | *mU | |
| G016016 | 707 | UUUCAA | UUUCAAAACCU | mU*mU*mU*CAAAACCUG | chr14: |
| | | AACCUG | GUCAGUGAUGU | UCAGUGAUGUUUUAGAm | 22550570- |
| | | UCAGUG | UUUAGAGCUAG | GmCmUmAmGmAmAmAm | 22550590 |
| | | AU | AAAUAGCAAGU | UmAmGmCAAGUUAAAAU | |
| | | | UAAAAUAAGGC | AAGGCUAGUCCGUUAUC | |
| | | | UAGUCCGUUAU | AmAmCmUmUmGmAmAm | |
| | | | CAACUUGAAAA | AmAmAmGmUmGmGmCm | |
| | | | AGUGGCACCGA | AmCmCmGmAmGmUmCm | |
| | | | GUCGGUGCUUU | GmGmUmGmCmU*mU*mU | |
| | | | U | *mU | |
| G016026 | 708 | CUUACC | CUUACCUGGGC | mC*mU*mU*ACCUGGGCU | chr14: |
| | | UGGGCU | UGGGGAAGAGU | GGGGAAGAGUUUUAGAm | 22547763- |
| | | GGGGA | UUUAGAGCUAG | GmCmUmAmGmAmAmAm | 22547783 |
| | | AGA | AAAUAGCAAGU | UmAmGmCAAGUUAAAAU | |
| | | | UAAAAUAAGGC | AAGGCUAGUCCGUUAUC | |
| | | | UAGUCCGUUAU | AmAmCmUmUmGmAmAm | |
| | | | CAACUUGAAAA | AmAmAmGmUmGmGmCm | |
| | | | AGUGGCACCGA | AmCmCmGmAmGmUmCm | |
| | | | GUCGGUGCUUU | GmGmUmGmCmU*mU*mU | |
| | | | U | *mU | |
| G016013 | 709 | CCGAAU | CCGAAUCCUCCU | mC*mC*mG*AAUCCUCCU | chr14: |
| | | CCUCCU | CCUGAAAGGUU | CCUGAAAGGUUUUAGAm | 22550596- |
| | | CCUGAA | UUAGAGCUAGA | GmCmUmAmGmAmAmAm | 22550616 |
| | | AG | AAUAGCAAGUU | UmAmGmCAAGUUAAAAU | |
| | | | AAAAUAAGGCU | AAGGCUAGUCCGUUAUC | |
| | | | AGUCCGUUAUC | AmAmCmUmUmGmAmAm | |
| | | | AACUUGAAAAA | AmAmAmGmUmGmGmCm | |
| | | | GUGGCACCGAG | AmCmCmGmAmGmUmCm | |
| | | | UCGGUGCUUUU | GmGmUmGmCmU*mU*mU | |
| | | | | *mU | |
| G016022 | 710 | CUGACA | CUGACAGGUUU | mC*mU*mG*ACAGGUUUU | chr14: |
| | | GGUUU | UGAAAGUUUGU | GAAAGUUUGUUUUAGAm | 22550566- |
| | | UGAAA | UUUAGAGCUAG | GmCmUmAmGmAmAmAm | 22550586 |
| | | GUUU | AAAUAGCAAGU | UmAmGmCAAGUUAAAAU | |
| | | | UAAAAUAAGGC | AAGGCUAGUCCGUUAUC | |
| | | | UAGUCCGUUAU | AmAmCmUmUmGmAmAm | |
| | | | CAACUUGAAAA | AmAmAmGmUmGmGmCm | |
| | | | AGUGGCACCGA | AmCmCmGmAmGmUmCm | |
| | | | GUCGGUGCUUU | GmGmUmGmCmU*mU*mU | |
| | | | U | *mU | |
| G016023 | 711 | CUGGGG | CUGGGGAAGAA | mC*mU*mG*GGGAAGAA | chr14: |
| | | AAGAA | GGUGUCUUCGU | GGUGUCUUCGUUUUAGA | 22547753- |
| | | GGUGUC | UUUAGAGCUAG | mGmCmUmAmGmAmAmA | 22547773 |
| | | UUC | AAAUAGCAAGU | mUmAmGmCAAGUUAAA | |
| | | | UAAAAUAAGGC | AUAAGGCUAGUCCGUUA | |
| | | | UAGUCCGUUAU | UCAmAmCmUmUmGmAm | |
| | | | CAACUUGAAAA | AmAmAmAmGmUmGmGm | |
| | | | AGUGGCACCGA | CmAmCmCmGmAmGmUm | |
| | | | GUCGGUGCUUU | CmGmGmUmGmCmU*mU* | |
| | | | U | mU*mU | |
| G016010 | 712 | UCCUCC | UCCUCCUCCUGA | mU*mC*mC*UCCUCCUGA | chr14: |
| | | UCCUGA | AAGUGGCCGUU | AAGUGGCCGUUUUAGAm | 22550601- |
| | | AAGUG | UUAGAGCUAGA | GmCmUmAmGmAmAmAm | 22550621 |
| | | GCC | AAUAGCAAGUU | UmAmGmCAAGUUAAAAU | |
| | | | AAAAUAAGGCU | AAGGCUAGUCCGUUAUC | |
| | | | AGUCCGUUAUC | AmAmCmUmUmGmAmAm | |
| | | | AACUUGAAAAA | AmAmAmGmUmGmGmCm | |
| | | | GUGGCACCGAG | AmCmCmGmAmGmUmCm | |
| | | | UCGGUGCUUUU | GmGmUmGmCmU*mU*mU | |
| | | | | *mU | |
| G016014 | 713 | CCACUU | CCACUUUCAGG | mC*mC*mA*CUUUCAGGA | chr14: |
| | | UCAGGA | AGGAGGAUUGU | GGAGGAUUGUUUUAGAm | 22550599- |
| | | GGAGG | UUUAGAGCUAG | GmCmUmAmGmAmAmAm | 22550619 |
| | | AUU | AAAUAGCAAGU | UmAmGmCAAGUUAAAAU | |
| | | | UAAAAUAAGGC | AAGGCUAGUCCGUUAUC | |
| | | | UAGUCCGUUAU | AmAmCmUmUmGmAmAm | |
| | | | CAACUUGAAAA | AmAmAmGmUmGmGmCm | |
| | | | AGUGGCACCGA | AmCmCmGmAmGmUmCm | |
| | | | GUCGGUGCUUU | GmGmUmGmCmU*mU*mU | |
| | | | U | *mU | |
| G016020 | 714 | AUUUG | AUUUGUUUGAG | mA*mU*mU*UGUUUGAG | chr14: |
| | | UUUGA | AAUCAAAAUGU | AAUCAAAAUGUUUUAGA | 22547583- |
| | | GAAUCA | UUUAGAGCUAG | mGmCmUmAmGmAmAmA | 22547603 |
| | | AAAU | AAAUAGCAAGU | mUmAmGmCAAGUUAAA | |
| | | | UAAAAUAAGGC | AUAAGGCUAGUCCGUUA | |
| | | | UAGUCCGUUAU | UCAmAmCmUmUmGmAm | |
| | | | CAACUUGAAAA | AmAmAmAmGmUmGmGm | |
| | | | AGUGGCACCGA | CmAmCmCmGmAmGmUm | |
| | | | GUCGGUGCUUU | CmGmGmUmGmCmU*mU* | |
| | | | U | mU*mU | |
| G015998 | 715 | CUUCAA | CUUCAAGAGCA | mC*mU*mU*CAAGAGCAA | chr14: |
| | | GAGCAA | ACAGUGCUGGU | CAGUGCUGGUUUUAGAm | 22547671- |
| | | CAGUGC | UUUAGAGCUAG | GmCmUmAmGmAmAmAm | 22547691 |
| | | UG | AAAUAGCAAGU | UmAmGmCAAGUUAAAAU | |
| | | | UAAAAUAAGGC | AAGGCUAGUCCGUUAUC | |
| | | | UAGUCCGUUAU | AmAmCmUmUmGmAmAm | |
| | | | CAACUUGAAAA | AmAmAmGmUmGmGmCm | |
| | | | AGUGGCACCGA | AmCmCmGmAmGmUmCm | |
| | | | GUCGGUGCUUU | GmGmUmGmCmU*mU*mU | |
| | | | U | *mU | |
| G016025 | 716 | AGCUGC | AGCUGCCCUUA | mA*mG*mC*UGCCCUUAC | chr14: |
| | | CCUUAC | CCUGGGCUGGU | CUGGGCUGGUUUUAGAm | 22547770- |
| | | CUGGGC | UUUAGAGCUAG | GmCmUmAmGmAmAmAm | 22547790 |
| | | UG | AAAUAGCAAGU | UmAmGmCAAGUUAAAAU | |
| | | | UAAAAUAAGGC | AAGGCUAGUCCGUUAUC | |
| | | | UAGUCCGUUAU | AmAmCmUmUmGmAmAm | |
| | | | CAACUUGAAAA | AmAmAmGmUmGmGmCm | |
| | | | AGUGGCACCGA | AmCmCmGmAmGmUmCm | |
| | | | GUCGGUGCUUU | GmGmUmGmCmU*mU*mU | |
| | | | U | *mU | |
| G015997 | 717 | AGAGCA | AGAGCAACAGU | mA*mG*mA*GCAACAGUG | chr14: |
| | | ACAGUG | GCUGUGGCCGU | CUGUGGCCGUUUUAGAm | 22547676- |
| | | CUGUGG | UUUAGAGCUAG | GmCmUmAmGmAmAmAm | 22547696 |
| | | CC | AAAUAGCAAGU | UmAmGmCAAGUUAAAAU | |
| | | | UAAAAUAAGGC | AAGGCUAGUCCGUUAUC | |
| | | | UAGUCCGUUAU | AmAmCmUmUmGmAmAm | |
| | | | CAACUUGAAAA | AmAmAmGmUmGmGmCm | |
| | | | AGUGGCACCGA | AmCmCmGmAmGmUmCm | |
| | | | GUCGGUGCUUU | GmGmUmGmCmU*mU*mU | |
| | | | U | *mU | |
| G016029 | 718 | AAAGCU | AAAGCUGCCCU | mA*mA*mA*GCUGCCCUU | chr14: |
| | | GCCCUU | UACCUGGGCGU | ACCUGGGCGUUUUAGAm | 22547772- |
| | | ACCUGG | UUUAGAGCUAG | GmCmUmAmGmAmAmAm | 22547792 |
| | | GC | AAAUAGCAAGU | UmAmGmCAAGUUAAAAU | |
| | | | UAAAAUAAGGC | AAGGCUAGUCCGUUAUC | |
| | | | UAGUCCGUUAU | AmAmCmUmUmGmAmAm | |
| | | | CAACUUGAAAA | AmAmAmGmUmGmGmCm | |
| | | | AGUGGCACCGA | AmCmCmGmAmGmUmCm | |
| | | | GUCGGUGCUUU | GmGmUmGmCmU*mU*mU | |
| | | | U | *mU | |
| G016028 | 719 | AAGCUG | AAGCUGCCCUU | mA*mA*mG*CUGCCCUUA | chr14: |
| | | CCCUUA | ACCUGGGCUGU | CCUGGGCUGUUUUAGAm | 22547771- |
| | | CCUGGG | UUUAGAGCUAG | GmCmUmAmGmAmAmAm | 22547791 |
| | | CU | AAAUAGCAAGU | UmAmGmCAAGUUAAAAU | |
| | | | UAAAAUAAGGC | AAGGCUAGUCCGUUAUC | |
| | | | UAGUCCGUUAU | AmAmCmUmUmGmAmAm | |
| | | | CAACUUGAAAA | AmAmAmGmUmGmGmCm | |
| | | | AGUGGCACCGA | AmCmCmGmAmGmUmCm | |
| | | | GUCGGUGCUUU | GmGmUmGmCmU*mU*mU | |
| | | | U | *mU | |
| G016021 | 720 | UGGAA | UGGAAUAAUGC | mU*mG*mG*AAUAAUGC | chr14: |
| | | UAAUGC | UGUUGUUGAGU | UGUUGUUGAGUUUUAGA | 22547733- |
| | | UGUUG | UUUAGAGCUAG | mGmCmUmAmGmAmAmA | 22547753 |
| | | UUGA | AAAUAGCAAGU | mUmAmGmCAAGUUAAA | |
| | | | UAAAAUAAGGC | AUAAGGCUAGUCCGUUA | |
| | | | UAGUCCGUUAU | UCAmAmCmUmUmGmAm | |
| | | | CAACUUGAAAA | AmAmAmAmGmUmGmGm | |
| | | | AGUGGCACCGA | CmAmCmCmGmAmGmUm | |
| | | | GUCGGUGCUUU | CmGmGmUmGmCmU*mU* | |
| | | | U | mU*mU | |
| G016015 | 721 | CACCAA | CACCAAAGCUG | mC*mA*mC*CAAAGCUGC | chr14: |
| | | AGCUGC | CCCUUACCUGU | CCUUACCUGUUUUAGAm | 22547776- |
| | | CCUUAC | UUUAGAGCUAG | GmCmUmAmGmAmAmAm | 22547796 |
| | | CU | AAAUAGCAAGU | UmAmGmCAAGUUAAAAU | |
| | | | UAAAAUAAGGC | AAGGCUAGUCCGUUAUC | |
| | | | UAGUCCGUUAU | AmAmCmUmUmGmAmAm | |
| | | | CAACUUGAAAA | AmAmAmGmUmGmGmCm | |
| | | | AGUGGCACCGA | AmCmCmGmAmGmUmCm | |
| | | | GUCGGUGCUUU | GmGmUmGmCmU*mU*mU | |
| | | | U | *mU | |

In some embodiments, the guide sequence comprises SEQ ID NO: 706. In some embodiments, the guide sequence comprises SEQ ID NO: 707. In some embodiments, the guide sequence comprises SEQ ID NO: 708. In some embodiments, the guide sequence comprises SEQ ID NO: 709. In some embodiments, the guide sequence comprises SEQ ID NO: 710. In some embodiments, the guide sequence comprises SEQ ID NO: 711. In some embodiments, the guide sequence comprises SEQ ID NO: 712. In some embodiments, the guide sequence comprises SEQ ID NO: 713. In some embodiments, the guide sequence comprises SEQ ID NO: 714. In some embodiments, the guide sequence comprises SEQ ID NO: 715. In some embodiments, the guide sequence comprises SEQ ID NO: 716. In some embodiments, the guide sequence comprises SEQ ID NO: 717. In some embodiments, the guide sequence comprises SEQ ID NO: 718. In some embodiments, the guide sequence comprises SEQ ID NO: 719. In some embodiments, the guide sequence comprises SEQ ID NO: 720. In some embodiments, the guide sequence comprises SEQ ID NO: 721.

In some embodiments, the gRNA targeting TRBC comprises a guide sequence chosen from: i) SEQ ID NOs: 618-669; ii) at least 17, 18, 19, or 20 contiguous nucleotides of a sequence selected from SEQ ID NOs: 618-669; iii) a guide sequence at least 95%, 90%, or 85% identical to a sequence selected from SEQ ID NOs: 618-669; iv) a sequence that comprises 10 contiguous nucleotides±10 nucleotides of a genomic coordinate listed in Table 5B; v) at least 17, 18, 19, or 20 contiguous nucleotides of a sequence from (iv); or vi) a guide sequence that is at least 95%, 90%, or 85% identical to a sequence selected from (v).

TABLE 5B
TRBC guide sequences, guide RNA sequences, and chromosomal
coordinates

| | SEQ ID | | Exemplary Full | | |
|---|---|---|---|---|---|
| | NO to the | | Sequence (SEQ | | Genomic |
| Guide | Guide | Guide | ID NOS: 1024- | Exemplary Mod Sequence | Coordinates |
| ID | Sequence | Sequence | 1075) | (SEQ ID NOS: 801-852) | (hg38) |
| G016200 | 618 | CCACAC | CCACACCCAAA | mC*mC*mA*CACCCAAA | chr7: |
| | | CCAAAA | AGGCCACACGU | AGGCCACACGUUUUAG | 142791757- |
| | | GGCCAC | UUUAGAGCUAG | AmGmCmUmAmGmAmAm | 142791777 |
| | | AC | AAAUAGCAAGU | AmUmAmGmCAAGUUAA | chr7: |
| | | | UAAAAUAAGGC | AAUAAGGCUAGUCCGU | 142801104- |
| | | | UAGUCCGUUAU | UAUCAmAmCmUmUmGm | 142801124 |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016174 | 619 | CCCACC | CCCACCAGCUC | mC*mC*mC*ACCAGCUC | chr7: |
| | | AGCUCA | AGCUCCACGGU | AGCUCCACGGUUUUAG | 142791811- |
| | | GCUCCA | UUUAGAGCUAG | AmGmCmUmAmGmAmAm | 142791831; |
| | | CG | AAAUAGCAAGU | AmUmAmGmCAAGUUAA | chr7: |
| | | | UAAAAUAAGGC | AAUAAGGCUAGUCCGU | 142801158- |
| | | | UAGUCCGUUAU | UAUCAmAmCmUmUmGm | 142801178 |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016263 | 620 | UCCCUA | UCCCUAGCAGG | mU*mC*mC*CUAGCAGG | chr7: |
| | | GCAGGA | AUCUCAUAGGU | AUCUCAUAGGUUUUAG | 142792728- |
| | | UCUCAU | UUUAGAGCUAG | AmGmCmUmAmGmAmAm | 142792748 |
| | | AG | AAAUAGCAAGU | AmUmAmGmCAAGUUAA | |
| | | | UAAAAUAAGGC | AAUAAGGCUAGUCCGU | |
| | | | UAGUCCGUUAU | UAUCAmAmCmUmUmGm | |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016270 | 621 | GGCUCA | GGCUCAAACAC | mG*mG*mC*UCAAACAC | chr7: |
| | | AACACA | AGCGACCUCGU | AGCGACCUCGUUUUAG | 142791719- |
| | | GCGACC | UUUAGAGCUAG | AmGmCmUmAmGmAmAm | 142791739 |
| | | UC | AAAUAGCAAGU | AmUmAmGmCAAGUUAA | |
| | | | UAAAAUAAGGC | AAUAAGGCUAGUCCGU | |
| | | | UAGUCCGUUAU | UAUCAmAmCmUmUmGm | |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016212 | 622 | GGCACA | GGCACACCAGU | mG*mG*mC*ACACCAGU | chr7: |
| | | CCAGUG | GUGGCCUUUGU | GUGGCCUUUGUUUUAG | 142791766- |
| | | UGGCCU | UUUAGAGCUAG | AmGmCmUmAmGmAmAm | 142791786; |
| | | UU | AAAUAGCAAGU | AmUmAmGmCAAGUUAA | chr7: |
| | | | UAAAAUAAGGC | AAUAAGGCUAGUCCGU | 142801113- |
| | | | UAGUCCGUUAU | UAUCAmAmCmUmUmGm | 142801133 |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016207 | 623 | AGGUG | AGGUGGCCGAG | mA*mG*mG*UGGCCGAG | chr7: |
| | | GCCGAG | ACCCUCAGGGU | ACCCUCAGGGUUUUAG | 142791928- |
| | | ACCCUC | UUUAGAGCUAG | AmGmCmUmAmGmAmAm | 142791948; |
| | | AGG | AAAUAGCAAGU | AmUmAmGmCAAGUUAA | chr7: |
| | | | UAAAAUAAGGC | AAUAAGGCUAGUCCGU | 142801275- |
| | | | UAGUCCGUUAU | UAUCAmAmCmUmUmGm | 142801295 |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016205 | 624 | ACCUGC | ACCUGCUCUAC | mA*mC*mC*UGCUCUAC | chr7: |
| | | UCUACC | CCCAGGCCUGU | CCCAGGCCUGUUUUAG | 142792062- |
| | | CCAGGC | UUUAGAGCUAG | AmGmCmUmAmGmAmAm | 142792082; |
| | | CU | AAAUAGCAAGU | AmUmAmGmCAAGUUAA | chr7: |
| | | | UAAAAUAAGGC | AAUAAGGCUAGUCCGU | 142801409- |
| | | | UAGUCCGUUAU | UAUCAmAmCmUmUmGm | 142801429 |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016245 | 625 | CAUAGA | CAUAGAGGAUG | mC*mA*mU*AGAGGAUG | chr7: |
| | | GGAUG | GUGGCAGACGU | GUGGCAGACGUUUUAG | 142792713- |
| | | GUGGCA | UUUAGAGCUAG | AmGmCmUmAmGmAmAm | 142792733; |
| | | GAC | AAAUAGCAAGU | AmUmAmGmCAAGUUAA | chr7: |
| | | | UAAAAUAAGGC | AAUAAGGCUAGUCCGU | 142802126- |
| | | | UAGUCCGUUAU | UAUCAmAmCmUmUmGm | 142802146 |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016232 | 626 | CCACGU | CCACGUGGAGC | mC*mC*mA*CGUGGAGC | chr7: |
| | | GGAGCU | UGAGCUGGUGU | UGAGCUGGUGUUUUAG | 142791808- |
| | | GAGCUG | UUUAGAGCUAG | AmGmCmUmAmGmAmAm | 142791828; |
| | | GU | AAAUAGCAAGU | AmUmAmGmCAAGUUAA | chr7: |
| | | | UAAAAUAAGGC | AAUAAGGCUAGUCCGU | 142801155- |
| | | | UAGUCCGUUAU | UAUCAmAmCmUmUmGm | 142801175 |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016227 | 627 | GGAGA | GGAGAAUGACG | mG*mG*mA*GAAUGACG | chr7: |
| | | AUGACG | AGUGGACCCGU | AGUGGACCCGUUUUAG | 142792003- |
| | | AGUGG | UUUAGAGCUAG | AmGmCmUmAmGmAmAm | 142792023; |
| | | ACCC | AAAUAGCAAGU | AmUmAmGmCAAGUUAA | chr7: |
| | | | UAAAAUAAGGC | AAUAAGGCUAGUCCGU | 142801350- |
| | | | UAGUCCGUUAU | UAUCAmAmCmUmUmGm | 142801370 |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016273 | 628 | CCAGUG | CCAGUGUGGCC | mC*mC*mA*GUGUGGCC | chr7: |
| | | UGGCCU | UUUUGGGUGG | UUUUGGGUGGUUUUAG | 142791760- |
| | | UUUGG | UUUUAGAGCUA | AmGmCmUmAmGmAmAm | 142791780 |
| | | GUG | GAAAUAGCAAG | AmUmAmGmCAAGUUAA | |
| | | | UUAAAAUAAG | AAUAAGGCUAGUCCGU | |
| | | | GCUAGUCCGUU | UAUCAmAmCmUmUmGm | |
| | | | AUCAACUUGAA | AmAmAmAmAmGmUmG | |
| | | | AAAGUGGCACC | mGmCmAmCmCmGmAmG | |
| | | | GAGUCGGUGCU | mUmCmGmGmUmGmCmU | |
| | | | UUU | *mU*mU*mU | |
| G016251 | 629 | CAAACA | CAAACACAGCG | mC*mA*mA*ACACAGCG | chr7: |
| | | CAGCGA | ACCUCGGGUGU | ACCUCGGGUGUUUUAG | 142791715- |
| | | CCUCGG | UUUAGAGCUAG | AmGmCmUmAmGmAmAm | 142791735 |
| | | GU | AAAUAGCAAGU | AmUmAmGmCAAGUUAA | |
| | | | UAAAAUAAGGC | AAUAAGGCUAGUCCGU | |
| | | | UAGUCCGUUAU | UAUCAmAmCmUmUmGm | |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016276 | 630 | UACCAU | UACCAUGGCCA | mU*mA*mC*CAUGGCCA | chr7: |
| | | GGCCAU | UCAACACAAGU | UCAACACAAGUUUUAG | 142792781- |
| | | CAACAC | UUUAGAGCUAG | AmGmCmUmAmGmAmAm | 142792801 |
| | | AA | AAAUAGCAAGU | AmUmAmGmCAAGUUAA | |
| | | | UAAAAUAAGGC | AAUAAGGCUAGUCCGU | |
| | | | UAGUCCGUUAU | UAUCAmAmCmUmUmGm | |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016167 | 631 | GCGCUG | GCGCUGACGAU | mG*mC*mG*CUGACGAU | chr7: |
| | | ACGAUC | CUGGGUGACGU | CUGGGUGACGUUUUAG | 142792040- |
| | | UGGGU | UUUAGAGCUAG | AmGmCmUmAmGmAmAm | 142792060; |
| | | GAC | AAAUAGCAAGU | AmUmAmGmCAAGUUAA | chr7: |
| | | | UAAAAUAAGGC | AAUAAGGCUAGUCCGU | 142801387- |
| | | | UAGUCCGUUAU | UAUCAmAmCmUmUmGm | 142801407 |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016253 | 632 | CACGGA | CACGGACCCGC | mC*mA*mC*GGACCCGC | chr7: |
| | | CCCGCA | AGCCCCUCAGU | AGCCCCUCAGUUUUAG | 142791862- |
| | | GCCCCU | UUUAGAGCUAG | AmGmCmUmAmGmAmAm | 142791882 |
| | | CA | AAAUAGCAAGU | AmUmAmGmCAAGUUAA | |
| | | | UAAAAUAAGGC | AAUAAGGCUAGUCCGU | |
| | | | UAGUCCGUUAU | UAUCAmAmCmUmUmGm | |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016272 | 633 | UCAAAC | UCAAACACAGC | mU*mC*mA*AACACAGC | chr7: |
| | | ACAGCG | GACCUCGGGGU | GACCUCGGGGUUUUAG | 142791716- |
| | | ACCUCG | UUUAGAGCUAG | AmGmCmUmAmGmAmAm | 142791736 |
| | | GG | AAAUAGCAAGU | AmUmAmGmCAAGUUAA | |
| | | | UAAAAUAAGGC | AAUAAGGCUAGUCCGU | |
| | | | UAGUCCGUUAU | UAUCAmAmCmUmUmGm | |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016258 | 634 | CAGGGA | CAGGGAAGAAG | mC*mA*mG*GGAAGAAG | chr7: |
| | | AGAAGC | CCUGUGGCCGU | CCUGUGGCCGUUUUAG | 142791787- |
| | | CUGUGG | UUUAGAGCUAG | AmGmCmUmAmGmAmAm | 142791807 |
| | | CC | AAAUAGCAAGU | AmUmAmGmCAAGUUAA | |
| | | | UAAAAUAAGGC | AAUAAGGCUAGUCCGU | |
| | | | UAGUCCGUUAU | UAUCAmAmCmUmUmGm | |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016183 | 635 | CAGUGU | CAGUGUGGCCU | mC*mA*mG*UGUGGCCU | chr7: |
| | | GGCCUU | UUUGGGUGUG | UUUGGGUGUGUUUUAG | 142791759- |
| | | UUGGG | UUUUAGAGCUA | AmGmCmUmAmGmAmAm | 142791779; |
| | | UGU | GAAAUAGCAAG | AmUmAmGmCAAGUUAA | chr7: |
| | | | UUAAAAUAAG | AAUAAGGCUAGUCCGU | 142801106- |
| | | | GCUAGUCCGUU | UAUCAmAmCmUmUmGm | 142801126 |
| | | | AUCAACUUGAA | AmAmAmAmAmGmUmG | |
| | | | AAAGUGGCACC | mGmCmAmCmCmGmAmG | |
| | | | GAGUCGGUGCU | mUmCmGmGmUmGmCmU | |
| | | | UUU | *mU*mU*mU | |
| G016222 | 636 | ACCACG | ACCACGUGGAG | mA*mC*mC*ACGUGGAG | chr7: |
| | | UGGAGC | CUGAGCUGGGU | CUGAGCUGGGUUUUAG | 142791807- |
| | | UGAGCU | UUUAGAGCUAG | AmGmCmUmAmGmAmAm | 142791827; |
| | | GG | AAAUAGCAAGU | AmUmAmGmCAAGUUAA | chr7: |
| | | | UAAAAUAAGGC | AAUAAGGCUAGUCCGU | 142801154- |
| | | | UAGUCCGUUAU | UAUCAmAmCmUmUmGm | 142801174 |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016233 | 637 | GAGGGC | GAGGGGGGCU | mG*mA*mG*GGCGGGCU | chr7: |
| | | GGGCUG | GCUCCUUGAGU | GCUCCUUGAGUUUUAG | 142791879- |
| | | CUCCUU | UUUAGAGCUAG | AmGmCmUmAmGmAmAm | 142791899; |
| | | GA | AAAUAGCAAGU | AmUmAmGmCAAGUUAA | chr7: |
| | | | UAAAAUAAGGC | AAUAAGGCUAGUCCGU | 142801226- |
| | | | UAGUCCGUUAU | UAUCAmAmCmUmUmGm | 142801246 |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016264 | 638 | AGCUCA | AGCUCAGCUCC | mA*mG*mC*UCAGCUCC | chr7: |
| | | GCUCCA | ACGUGGUCAGU | ACGUGGUCAGUUUUAG | 142791805- |
| | | CGUGGU | UUUAGAGCUAG | AmGmCmUmAmGmAmAm | 142791825 |
| | | CA | AAAUAGCAAGU | AmUmAmGmCAAGUUAA | |
| | | | UAAAAUAAGGC | AAUAAGGCUAGUCCGU | |
| | | | UAGUCCGUUAU | UAUCAmAmCmUmUmGm | |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016255 | 639 | GAACAA | GAACAAGGUGU | mG*mA*mA*CAAGGUGU | chr7: |
| | | GGUGU | UCCCACCCGGU | UCCCACCCGGUUUUAGA | 142791700- |
| | | UCCCAC | UUUAGAGCUAG | mGmCmUmAmGmAmAmA | 142791720 |
| | | CCG | AAAUAGCAAGU | mUmAmGmCAAGUUAAA | |
| | | | UAAAAUAAGGC | AUAAGGCUAGUCCGUU | |
| | | | UAGUCCGUUAU | AUCAmAmCmUmUmGmA | |
| | | | CAACUUGAAAA | mAmAmAmAmGmUmGm | |
| | | | AGUGGCACCGA | GmCmAmCmCmGmAmGm | |
| | | | GUCGGUGCUUU | UmCmGmGmUmGmCmU* | |
| | | | U | mU*mU*mU | |
| G016177 | 640 | GCACAC | GCACACCAGUG | mG*mC*mA*CACCAGUG | chr7: |
| | | CAGUGU | UGGCCUUUUGU | UGGCCUUUUGUUUUAG | 142791765- |
| | | GGCCUU | UUUAGAGCUAG | AmGmCmUmAmGmAmAm | 142791785; |
| | | UU | AAAUAGCAAGU | AmUmAmGmCAAGUUAA | chr7: |
| | | | UAAAAUAAGGC | AAUAAGGCUAGUCCGU | 142801112- |
| | | | UAGUCCGUUAU | UAUCAmAmCmUmUmGm | 142801132 |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016283 | 641 | GAGCUG | GAGCUGGUGGG | mG*mA*mG*CUGGUGGG | chr7: |
| | | GUGGG | UGAAUGGGAG | UGAAUGGGAGUUUUAG | 142791820- |
| | | UGAAU | UUUUAGAGCUA | AmGmCmUmAmGmAmAm | 142791840 |
| | | GGGA | GAAAUAGCAAG | AmUmAmGmCAAGUUAA | |
| | | | UUAAAAUAAG | AAUAAGGCUAGUCCGU | |
| | | | GCUAGUCCGUU | UAUCAmAmCmUmUmGm | |
| | | | AUCAACUUGAA | AmAmAmAmAmGmUmG | |
| | | | AAAGUGGCACC | mGmCmAmCmCmGmAmG | |
| | | | GAGUCGGUGCU | mUmCmGmGmUmGmCmU | |
| | | | UUU | *mU*mU*mU | |
| G016194 | 642 | GGCUGC | GGCUGCUCCUU | mG*mG*mC*UGCUCCUU | chr7: |
| | | UCCUUG | GAGGGGCUGGU | GAGGGGCUGGUUUUAG | 142791872- |
| | | AGGGGC | UUUAGAGCUAG | AmGmCmUmAmGmAmAm | 142791892; |
| | | UG | AAAUAGCAAGU | AmUmAmGmCAAGUUAA | chr7: |
| | | | UAAAAUAAGGC | AAUAAGGCUAGUCCGU | 142801219- |
| | | | UAGUCCGUUAU | UAUCAmAmCmUmUmGm | 142801239 |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016266 | 643 | CGGGUG | CGGGUGGGAAC | mC*mG*mG*GUGGGAAC | chr7: |
| | | GGAACA | ACCUUGUUCGU | ACCUUGUUCGUUUUAG | 142791700- |
| | | CCUUGU | UUUAGAGCUAG | AmGmCmUmAmGmAmAm | 142791720 |
| | | UC | AAAUAGCAAGU | AmUmAmGmCAAGUUAA | |
| | | | UAAAAUAAGGC | AAUAAGGCUAGUCCGU | |
| | | | UAGUCCGUUAU | UAUCAmAmCmUmUmGm | |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016196 | 644 | CAGCUC | CAGCUCAGCUC | mC*mA*mG*CUCAGCUC | chr7: |
| | | AGCUCC | CACGUGGUCGU | CACGUGGUCGUUUUAG | 142791806- |
| | | ACGUGG | UUUAGAGCUAG | AmGmCmUmAmGmAmAm | 142791826; |
| | | UC | AAAUAGCAAGU | AmUmAmGmCAAGUUAA | chr7: |
| | | | UAAAAUAAGGC | AAUAAGGCUAGUCCGU | 142801153- |
| | | | UAGUCCGUUAU | UAUCAmAmCmUmUmGm | 142801173 |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016274 | 645 | GACGAU | GACGAUCUGGG | mG*mA*mC*GAUCUGGG | chr7: |
| | | CUGGGU | UGACGGGUUGU | UGACGGGUUGUUUUAG | 142792035- |
| | | GACGGG | UUUAGAGCUAG | AmGmCmUmAmGmAmAm | 142792055 |
| | | UU | AAAUAGCAAGU | AmUmAmGmCAAGUUAA | |
| | | | UAAAAUAAGGC | AAUAAGGCUAGUCCGU | |
| | | | UAGUCCGUUAU | UAUCAmAmCmUmUmGm | |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016282 | 646 | UAGCAG | UAGCAGGAUCU | mU*mA*mG*CAGGAUCU | chr7: |
| | | GAUCUC | CAUAGAGGAGU | CAUAGAGGAGUUUUAG | 142792724- |
| | | AUAGA | UUUAGAGCUAG | AmGmCmUmAmGmAmAm | 142792744 |
| | | GGA | AAAUAGCAAGU | AmUmAmGmCAAGUUAA | |
| | | | UAAAAUAAGGC | AAUAAGGCUAGUCCGU | |
| | | | UAGUCCGUUAU | UAUCAmAmCmUmUmGm | |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016267 | 647 | GACCAG | GACCAGCACAG | mG*mA*mC*CAGCACAG | chr7: |
| | | CACAGC | CAUACAGGGGU | CAUACAGGGGUUUUAG | 142792754- |
| | | AUACAG | UUUAGAGCUAG | AmGmCmUmAmGmAmAm | 142792774 |
| | | GG | AAAUAGCAAGU | AmUmAmGmCAAGUUAA | |
| | | | UAAAAUAAGGC | AAUAAGGCUAGUCCGU | |
| | | | UAGUCCGUUAU | UAUCAmAmCmUmUmGm | |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016261 | 648 | CUGACC | CUGACCACGUG | mC*mU*mG*ACCACGUG | chr7: |
| | | ACGUGG | GAGCUGAGCGU | GAGCUGAGCGUUUUAG | 142791804- |
| | | AGCUGA | UUUAGAGCUAG | AmGmCmUmAmGmAmAm | 142791824 |
| | | GC | AAAUAGCAAGU | AmUmAmGmCAAGUUAA | |
| | | | UAAAAUAAGGC | AAUAAGGCUAGUCCGU | |
| | | | UAGUCCGUUAU | UAUCAmAmCmUmUmGm | |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016277 | 649 | CCAACA | CCAACAGUGUC | mC*mC*mA*ACAGUGUC | chr7: |
| | | GUGUCC | CUACCAGCAGU | CUACCAGCAGUUUUAG | 142792684- |
| | | UACCAG | UUUAGAGCUAG | AmGmCmUmAmGmAmAm | 142792704 |
| | | CA | AAAUAGCAAGU | AmUmAmGmCAAGUUAA | |
| | | | UAAAAUAAGGC | AAUAAGGCUAGUCCGU | |
| | | | UAGUCCGUUAU | UAUCAmAmCmUmUmGm | |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016285 | 650 | CUGGUG | CUGGUGGGUGA | mC*mU*mG*GUGGGUGA | chr7: |
| | | GGUGA | AUGGGAAGGG | AUGGGAAGGGUUUUAG | 142791823- |
| | | AUGGG | UUUUAGAGCUA | AmGmCmUmAmGmAmAm | 142791843 |
| | | AAGG | GAAAUAGCAAG | AmUmAmGmCAAGUUAA | |
| | | | UUAAAAUAAG | AAUAAGGCUAGUCCGU | |
| | | | GCUAGUCCGUU | UAUCAmAmCmUmUmGm | |
| | | | AUCAACUUGAA | AmAmAmAmAmGmUmG | |
| | | | AAAGUGGCACC | mGmCmAmCmCmGmAmG | |
| | | | GAGUCGGUGCU | mUmCmGmGmUmGmCmU | |
| | | | UUU | *mU*mU*mU | |
| G016275 | 651 | CUAUGA | CUAUGAGAUCC | mC*mU*mA*UGAGAUCC | chr7: |
| | | GAUCCU | UGCUAGGGAGU | UGCUAGGGAGUUUUAG | 142792728- |
| | | GCUAGG | UUUAGAGCUAG | AmGmCmUmAmGmAmAm | 142792748 |
| | | GA | AAAUAGCAAGU | AmUmAmGmCAAGUUAA | |
| | | | UAAAAUAAGGC | AAUAAGGCUAGUCCGU | |
| | | | UAGUCCGUUAU | UAUCAmAmCmUmUmGm | |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016281 | 652 | CAGGAU | CAGGAUCUCAU | mC*mA*mG*GAUCUCAU | chr7: |
| | | CUCAUA | AGAGGAUGGG | AGAGGAUGGGUUUUAG | 142792721- |
| | | GAGGA | UUUUAGAGCUA | AmGmCmUmAmGmAmAm | 142792741 |
| | | UGG | GAAAUAGCAAG | AmUmAmGmCAAGUUAA | |
| | | | UUAAAAUAAG | AAUAAGGCUAGUCCGU | |
| | | | GCUAGUCCGUU | UAUCAmAmCmUmUmGm | |
| | | | AUCAACUUGAA | AmAmAmAmAmGmUmG | |
| | | | AAAGUGGCACC | mGmCmAmCmCmGmAmG | |
| | | | GAGUCGGUGCU | mUmCmGmGmUmGmCmU | |
| | | | UUU | *mU*mU*mU | |
| G016280 | 653 | GGCCAC | GGCCACCCUGU | mG*mG*mC*CACCCUGU | chr7: |
| | | CCUGUA | AUGCUGUGCGU | AUGCUGUGCGUUUUAG | 142792749- |
| | | UGCUGU | UUUAGAGCUAG | AmGmCmUmAmGmAmAm | 142792769 |
| | | GC | AAAUAGCAAGU | AmUmAmGmCAAGUUAA | |
| | | | UAAAAUAAGGC | AAUAAGGCUAGUCCGU | |
| | | | UAGUCCGUUAU | UAUCAmAmCmUmUmGm | |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016279 | 654 | CAACAG | CAACAGUGUCC | mC*mA*mA*CAGUGUCC | chr7: |
| | | UGUCCU | UACCAGCAAGU | UACCAGCAAGUUUUAG | 142792685- |
| | | ACCAGC | UUUAGAGCUAG | AmGmCmUmAmGmAmAm | 142792705 |
| | | AA | AAAUAGCAAGU | AmUmAmGmCAAGUUAA | |
| | | | UAAAAUAAGGC | AAUAAGGCUAGUCCGU | |
| | | | UAGUCCGUUAU | UAUCAmAmCmUmUmGm | |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016250 | 655 | AGCUGA | AGCUGAGCUGG | mA*mG*mC*UGAGCUGG | chr7: |
| | | GCUGGU | UGGGUGAAUG | UGGGUGAAUGUUUUAG | 142791816- |
| | | GGGUG | UUUUAGAGCUA | AmGmCmUmAmGmAmAm | 142791836; |
| | | AAU | GAAAUAGCAAG | AmUmAmGmCAAGUUAA | chr7: |
| | | | UUAAAAUAAG | AAUAAGGCUAGUCCGU | 142801163- |
| | | | GCUAGUCCGUU | UAUCAmAmCmUmUmGm | 142801183 |
| | | | AUCAACUUGAA | AmAmAmAmAmGmUmG | |
| | | | AAAGUGGCACC | mGmCmAmCmCmGmAmG | |
| | | | GAGUCGGUGCU | mUmCmGmGmUmGmCmU | |
| | | | UUU | *mU*mU*mU | |
| G016278 | 656 | AACAGU | AACAGUGUCCU | mA*mA*mC*AGUGUCCU | chr7: |
| | | GUCCUA | ACCAGCAAGGU | ACCAGCAAGGUUUUAG | 142792686- |
| | | CCAGCA | UUUAGAGCUAG | AmGmCmUmAmGmAmAm | 142792706 |
| | | AG | AAAUAGCAAGU | AmUmAmGmCAAGUUAA | |
| | | | UAAAAUAAGGC | AAUAAGGCUAGUCCGU | |
| | | | UAGUCCGUUAU | UAUCAmAmCmUmUmGm | |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016259 | 657 | AGGCUU | AGGCUUCUUCC | mA*mG*mG*CUUCUUCC | chr7: |
| | | CUUCCC | CUGACCACGGU | CUGACCACGGUUUUAG | 142791793- |
| | | UGACCA | UUUAGAGCUAG | AmGmCmUmAmGmAmAm | 142791813 |
| | | CG | AAAUAGCAAGU | AmUmAmGmCAAGUUAA | |
| | | | UAAAAUAAGGC | AAUAAGGCUAGUCCGU | |
| | | | UAGUCCGUUAU | UAUCAmAmCmUmUmGm | |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016284 | 658 | UCUUCU | UCUUCUGCAGG | mU*mC*mU*UCUGCAGG | chr7: |
| | | GCAGGU | UCAAGAGAAGU | UCAAGAGAAGUUUUAG | 142793110- |
| | | CAAGAG | UUUAGAGCUAG | AmGmCmUmAmGmAmAm | 142793130 |
| | | AA | AAAUAGCAAGU | AmUmAmGmCAAGUUAA | |
| | | | UAAAAUAAGGC | AAUAAGGCUAGUCCGU | |
| | | | UAGUCCGUUAU | UAUCAmAmCmUmUmGm | |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016249 | 659 | GAGCUG | GAGCUGAGCUG | mG*mA*mG*CUGAGCUG | chr7: |
| | | AGCUGG | GUGGGUGAAG | GUGGGUGAAGUUUUAG | 142791815- |
| | | UGGGU | UUUUAGAGCUA | AmGmCmUmAmGmAmAm | 142791835; |
| | | GAA | GAAAUAGCAAG | AmUmAmGmCAAGUUAA | chr7: |
| | | | UUAAAAUAAG | AAUAAGGCUAGUCCGU | 142801162- |
| | | | GCUAGUCCGUU | UAUCAmAmCmUmUmGm | 142801182 |
| | | | AUCAACUUGAA | AmAmAmAmAmGmUmG | |
| | | | AAAGUGGCACC | mGmCmAmCmCmGmAmG | |
| | | | GAGUCGGUGCU | mUmCmGmGmUmGmCmU | |
| | | | UUU | *mU*mU*mU | |
| G016256 | 660 | GGUCAG | GGUCAGCGCCC | mG*mG*mU*CAGCGCCC | chr7: |
| | | CGCCCU | UUGUGUUGAG | UUGUGUUGAGUUUUAG | 142792770- |
| | | UGUGU | UUUUAGAGCUA | AmGmCmUmAmGmAmAm | 142792790 |
| | | UGA | GAAAUAGCAAG | AmUmAmGmCAAGUUAA | |
| | | | UUAAAAUAAG | AAUAAGGCUAGUCCGU | |
| | | | GCUAGUCCGUU | UAUCAmAmCmUmUmGm | |
| | | | AUCAACUUGAA | AmAmAmAmAmGmUmG | |
| | | | AAAGUGGCACC | mGmCmAmCmCmGmAmG | |
| | | | GAGUCGGUGCU | mUmCmGmGmUmGmCmU | |
| | | | UUU | *mU*mU*mU | |
| G016190 | 661 | AGAUCG | AGAUCGUCAGC | mA*mG*mA*UCGUCAGC | chr7: |
| | | UCAGCG | GCCGAGGCCGU | GCCGAGGCCGUUUUAG | 142792047- |
| | | CCGAGG | UUUAGAGCUAG | AmGmCmUmAmGmAmAm | 142792067; |
| | | CC | AAAUAGCAAGU | AmUmAmGmCAAGUUAA | chr7: |
| | | | UAAAAUAAGGC | AAUAAGGCUAGUCCGU | 142801394- |
| | | | UAGUCCGUUAU | UAUCAmAmCmUmUmGm | 142801414 |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016248 | 662 | GCUGCU | GCUGCUCCUUG | mG*mC*mU*GCUCCUUG | chr7: |
| | | CCUUGA | AGGGGCUGCGU | AGGGGCUGCGUUUUAG | 142791871- |
| | | GGGGCU | UUUAGAGCUAG | AmGmCmUmAmGmAmAm | 142791891; |
| | | GC | AAAUAGCAAGU | AmUmAmGmCAAGUUAA | chr7: |
| | | | UAAAAUAAGGC | AAUAAGGCUAGUCCGU | 142801218- |
| | | | UAGUCCGUUAU | UAUCAmAmCmUmUmGm | 142801238 |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016257 | 663 | GUAUCU | GUAUCUGGAGU | mG*mU*mA*UCUGGAGU | chr7: |
| | | GGAGUC | CAUUGAGGGGU | CAUUGAGGGGUUUUAG | 142791894- |
| | | AUUGA | UUUAGAGCUAG | AmGmCmUmAmGmAmAm | 142791914 |
| | | GGG | AAAUAGCAAGU | AmUmAmGmCAAGUUAA | |
| | | | UAAAAUAAGGC | AAUAAGGCUAGUCCGU | |
| | | | UAGUCCGUUAU | UAUCAmAmCmUmUmGm | |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016260 | 664 | AUCCUC | AUCCUCUAUGA | mA*mU*mC*CUCUAUGA | chr7: |
| | | UAUGA | GAUCCUGCUGU | GAUCCUGCUGUUUUAG | 142792723- |
| | | GAUCCU | UUUAGAGCUAG | AmGmCmUmAmGmAmAm | 142792743 |
| | | GCU | AAAUAGCAAGU | AmUmAmGmCAAGUUAA | |
| | | | UAAAAUAAGGC | AAUAAGGCUAGUCCGU | |
| | | | UAGUCCGUUAU | UAUCAmAmCmUmUmGm | |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016262 | 665 | UCCUCU | UCCUCUAUGAG | mU*mC*mC*UCUAUGAG | chr7: |
| | | AUGAG | AUCCUGCUAGU | AUCCUGCUAGUUUUAG | 142792724- |
| | | AUCCUG | UUUAGAGCUAG | AmGmCmUmAmGmAmAm | 142792744 |
| | | CUA | AAAUAGCAAGU | AmUmAmGmCAAGUUAA | |
| | | | UAAAAUAAGGC | AAUAAGGCUAGUCCGU | |
| | | | UAGUCCGUUAU | UAUCAmAmCmUmUmGm | |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016242 | 666 | GCAGUA | GCAGUAUCUGG | mG*mC*mA*GUAUCUGG | chr7: |
| | | UCUGGA | AGUCAUUGAGU | AGUCAUUGAGUUUUAG | 142791897- |
| | | GUCAUU | UUUAGAGCUAG | AmGmCmUmAmGmAmAm | 142791917; |
| | | GA | AAAUAGCAAGU | AmUmAmGmCAAGUUAA | chr7: |
| | | | UAAAAUAAGGC | AAUAAGGCUAGUCCGU | 142801244- |
| | | | UAGUCCGUUAU | UAUCAmAmCmUmUmGm | 142801264 |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016268 | 667 | GCUGAC | GCUGACCAGCA | mG*mC*mU*GACCAGCA | chr7: |
| | | CAGCAC | CAGCAUACAGU | CAGCAUACAGUUUUAG | 142792757- |
| | | AGCAUA | UUUAGAGCUAG | AmGmCmUmAmGmAmAm | 142792777 |
| | | CA | AAAUAGCAAGU | AmUmAmGmCAAGUUAA | |
| | | | UAAAAUAAGGC | AAUAAGGCUAGUCCGU | |
| | | | UAGUCCGUUAU | UAUCAmAmCmUmUmGm | |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016265 | 668 | ACAGGG | ACAGGGUGGCC | mA*mC*mA*GGGUGGCC | chr7: |
| | | UGGCCU | UUCCCUAGCGU | UUCCCUAGCGUUUUAG | 142792740- |
| | | UCCCUA | UUUAGAGCUAG | AmGmCmUmAmGmAmAm | 142792760 |
| | | GC | AAAUAGCAAGU | AmUmAmGmCAAGUUAA | |
| | | | UAAAAUAAGGC | AAUAAGGCUAGUCCGU | |
| | | | UAGUCCGUUAU | UAUCAmAmCmUmUmGm | |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |
| G016254 | 669 | CGCUGA | CGCUGACCAGC | mC*mG*mC*UGACCAGC | chr7: |
| | | CCAGCA | ACAGCAUACGU | ACAGCAUACGUUUUAG | 142792758- |
| | | CAGCAU | UUUAGAGCUAG | AmGmCmUmAmGmAmAm | 142792778 |
| | | AC | AAAUAGCAAGU | AmUmAmGmCAAGUUAA | |
| | | | UAAAAUAAGGC | AAUAAGGCUAGUCCGU | |
| | | | UAGUCCGUUAU | UAUCAmAmCmUmUmGm | |
| | | | CAACUUGAAAA | AmAmAmAmAmGmUmG | |
| | | | AGUGGCACCGA | mGmCmAmCmCmGmAmG | |
| | | | GUCGGUGCUUU | mUmCmGmGmUmGmCmU | |
| | | | U | *mU*mU*mU | |

In some embodiments, the guide sequence comprises SEQ ID NO: 618. In some embodiments, the guide sequence comprises SEQ ID NO: 619. In some embodiments, the guide sequence comprises SEQ ID NO: 620. In some embodiments, the guide sequence comprises SEQ ID NO: 621. In some embodiments, the guide sequence comprises SEQ ID NO: 622. In some embodiments, the guide sequence comprises SEQ ID NO: 623. In some embodiments, the guide sequence comprises SEQ ID NO: 624. In some embodiments, the guide sequence comprises SEQ ID NO: 625. In some embodiments, the guide sequence comprises SEQ ID NO: 626. In some embodiments, the guide sequence comprises SEQ ID NO: 627. In some embodiments, the guide sequence comprises SEQ ID NO: 628. In some embodiments, the guide sequence comprises SEQ ID NO: 629. In some embodiments, the guide sequence comprises SEQ ID NO: 630. In some embodiments, the guide sequence comprises SEQ ID NO: 631. In some embodiments, the guide sequence comprises SEQ ID NO: 632. In some embodiments, the guide sequence comprises SEQ ID NO: 633. In some embodiments, the guide sequence comprises SEQ ID NO: 634. In some embodiments, the guide sequence comprises SEQ ID NO: 635. In some embodiments, the guide sequence comprises SEQ ID NO: 636. In some embodiments, the guide sequence comprises SEQ ID NO: 637. In some embodiments, the guide sequence comprises SEQ ID NO: 638. In some embodiments, the guide sequence comprises SEQ ID NO: 639. In some embodiments, the guide sequence comprises SEQ ID NO: 640. In some embodiments, the guide sequence comprises SEQ ID NO: 641. In some embodiments, the guide sequence comprises SEQ ID NO: 642. In some embodiments, the guide sequence comprises SEQ ID NO: 643. In some embodiments, the guide sequence comprises SEQ ID NO: 644. In some embodiments, the guide sequence comprises SEQ ID NO: 645. In some embodiments, the guide sequence comprises SEQ ID NO: 646. In some embodiments, the guide sequence comprises SEQ ID NO: 647. In some embodiments, the guide sequence comprises SEQ ID NO: 648. In some embodiments, the guide sequence comprises SEQ ID NO: 649. In some embodiments, the guide sequence comprises SEQ ID NO: 650. In some embodiments, the guide sequence comprises SEQ ID NO: 651. In some embodiments, the guide sequence comprises SEQ ID NO: 652. In some embodiments, the guide sequence comprises SEQ ID NO: 653. In some embodiments, the guide sequence comprises SEQ ID NO: 654. In some embodiments, the guide sequence comprises SEQ ID NO: 655. In some embodiments, the guide sequence comprises SEQ ID NO: 656. In some embodiments, the guide sequence comprises SEQ ID NO: 657. In some embodiments, the guide sequence comprises SEQ ID NO: 658. In some embodiments, the guide sequence comprises SEQ ID NO: 659. In some embodiments, the guide sequence comprises SEQ ID NO: 660. In some embodiments, the guide sequence comprises SEQ ID NO: 661. In some embodiments, the guide sequence comprises SEQ ID NO: 662. In some embodiments, the guide sequence comprises SEQ ID NO: 663. In some embodiments, the guide sequence comprises SEQ ID NO: 664. In some embodiments, the guide sequence comprises SEQ ID NO: 665. In some embodiments, the guide sequence comprises SEQ ID NO: 666. In some embodiments, the guide sequence comprises SEQ ID NO: 667. In some embodiments, the guide sequence comprises SEQ ID NO: 668. In some embodiments, the guide sequence comprises SEQ ID NO: 669.

In some embodiments, the disclosure provides a method of altering a DNA sequence within a TRAC gene, comprising delivering a composition disclosed herein to a cell. The composition may comprise:
a. a gRNA comprising a guide sequence chosen from: i) SEQ ID NOs: 706-721; ii) at least 17, 18, 19, or 20 contiguous nucleotides of a sequence selected from SEQ ID NOs: 706-721; iii) a guide sequence at least 95%, 90%, or 85% identical to a sequence selected from SEQ ID NOs: 706-721; iv) a sequence that comprises 10 contiguous nucleotides±10 nucleotides of a genomic coordinate listed in Table 5A; v) at least 17, 18, 19, or 20 contiguous nucleotides of a sequence from (iv); or vi) a guide sequence that is at least 95%, 90%, or 85% identical to a sequence selected from (v); or
b. a nucleic acid encoding a gRNA of (a.).

In some embodiments, the disclosure provides a method of reducing the expression of a TRAC gene, comprising delivering a composition disclosed herein to a cell. The composition may comprise:
a. a gRNA comprising a guide sequence chosen from: i) SEQ ID NOs: 706-721; ii) at least 17, 18, 19, or 20 contiguous nucleotides of a sequence selected from SEQ ID NOs: 706-721; iii) a guide sequence at least 95%, 90%, or 85% identical to a sequence selected from SEQ ID NOs: 706-721; iv) a sequence that comprises 10 contiguous nucleotides±10 nucleotides of a genomic coordinate listed in Table 5A; v) at least 17, 18, 19, or 20 contiguous nucleotides of a sequence from (iv); or vi) a guide sequence that is at least 95%, 90%, or 85% identical to a sequence selected from (v); or
b. a nucleic acid encoding a gRNA of (a.).

In some embodiments, the disclosure provides a method of immunotherapy comprising administering a composition disclosed herein to a subject, an autologous cell thereof, and/or an allogeneic cell. The composition may comprise:
a. a gRNA comprising a guide sequence chosen from: i) SEQ ID NOs: 706-721; ii) at least 17, 18, 19, or 20 contiguous nucleotides of a sequence selected from SEQ ID NOs: 706-721; iii) a guide sequence at least 95%, 90%, or 85% identical to a sequence selected from SEQ ID NOs: 706-721; iv) a sequence that comprises 10 contiguous nucleotides±10 nucleotides of a genomic coordinate listed in Table 5A; v) at least 17, 18, 19, or 20 contiguous nucleotides of a sequence from (iv); or vi) a guide sequence that is at least 95%, 90%, or 85% identical to a sequence selected from (v); or
b. a nucleic acid encoding a gRNA of (a.).

In some embodiments, a cell altered by the method disclosed herein is provided. The cell may be altered ex vivo. The cell may be a T cell, a CD4⁺ or CD8⁺ cell. The cell may be a mammalian, primate, or human cell. The cell may be used for immunotherapy of a subject.

In some embodiments, a compositions is provided, comprising: a gRNA comprising a guide sequence chosen from: i) SEQ ID NOs: 706-721; ii) at least 17, 18, 19, or 20 contiguous nucleotides of a sequence selected from SEQ ID NOs: 706-721; iii) a guide sequence at least 95%, 90%, or 85% identical to a sequence selected from SEQ ID NOs: 706-721; iv) a sequence that comprises 10 contiguous nucleotides±10 nucleotides of a genomic coordinate listed in Table 5A; v) at least 17, 18, 19, or 20 contiguous nucleotides of a sequence from (iv); or vi) a guide sequence that is at least 95%, 90%, or 85% identical to a sequence selected from (v). The composition may optionally further comprise any one of a nucleic acid (e.g., mRNA), polypeptide, composition, or lipid nucleic acid assembly composition disclosed herein.

In certain embodiments, the composition disclosed herein is used for altering a DNA sequence within the TRAC gene in a cell. In certain embodiments, the composition disclosed herein is used for reducing the expression of the TRAC gene in a cell. In some embodiments, the composition disclosed herein is used for immunotherapy of a subject.

In some embodiments, the disclosure provides a method of altering a DNA sequence within a TRBC1 and/or TRBC2 gene, comprising delivering a composition disclosed herein to a cell. The composition may comprise:
a. a gRNA comprising a guide sequence chosen from: i) SEQ ID NOs: 618-669; ii) at least 17, 18, 19, or 20 contiguous nucleotides of a sequence selected from SEQ ID NOs: 618-669; iii) a guide sequence at least 95%, 90%, or 85% identical to a sequence selected from SEQ ID NOs: 618-669; iv) a sequence that comprises 10 contiguous nucleotides±10 nucleotides of a genomic coordinate listed in Table 5B; v) at least 17, 18, 19, or 20 contiguous nucleotides of a sequence from (iv); or vi) a guide sequence that is at least 95%, 90%, or 85% identical to a sequence selected from (v); or
b. a nucleic acid encoding a gRNA of (a.).

In some embodiments, the disclosure provides a method of reducing the expression of a TRBC1 and/or TRBC2 gene, comprising delivering a composition disclosed herein to a cell. The composition may comprise:
a. a gRNA comprising a guide sequence chosen from: i) SEQ ID NOs: 618-669; ii) at least 17, 18, 19, or 20 contiguous nucleotides of a sequence selected from SEQ ID NOs: 618-669; iii) a guide sequence at least 95%, 90%, or 85% identical to a sequence selected from SEQ ID NOs: 618-669; iv) a sequence that comprises 10 contiguous nucleotides±10 nucleotides of a genomic coordinate listed in Table 5C; v) at least 17, 18, 19, or 20 contiguous nucleotides of a sequence from (iv); or vi) a guide sequence that is at least 95%, 90%, or 85% identical to a sequence selected from (v); or
b. a nucleic acid encoding a gRNA of (a.).

In some embodiments, the disclosure provides a method of immunotherapy comprising administering a composition disclosed herein to a subject, an autologous cell thereof, and/or an allogeneic cell. The composition may comprise:
a. a gRNA comprising a guide sequence chosen from: i) SEQ ID NOs: 618-669; ii) at least 17, 18, 19, or 20 contiguous nucleotides of a sequence selected from SEQ ID NOs: 618-669; iii) a guide sequence at least 95%, 90%, or 85% identical to a sequence selected from SEQ ID NOs: 618-669; iv) a sequence that comprises 10 contiguous nucleotides±10 nucleotides of a genomic coordinate listed in Table 5B; v) at least 17, 18, 19, or 20 contiguous nucleotides of a sequence from (iv); or vi) a guide sequence that is at least 95%, 90%, or 85% identical to a sequence selected from (v); or
b. a nucleic acid encoding a gRNA of (a.).

In some embodiments, a cell may be provided, being altered by the method disclosed herein. The cell may be altered ex vivo. The cell is a T cell, a CD4⁺ or CD8⁺ cell. The cell may be a mammalian, primate, or human cell. The cell may be used for immunotherapy of a subject.

In some embodiments, a compositions is provided, comprising: a gRNA comprising a guide sequence chosen from: i) SEQ ID NOs: 618-669; ii) at least 17, 18, 19, or 20 contiguous nucleotides of a sequence selected from SEQ ID NOs: 618-669; iii) a guide sequence at least 95%, 90%, or 85% identical to a sequence selected from SEQ ID NOs: 618-669; iv) a sequence that comprises 10 contiguous nucleotides±10 nucleotides of a genomic coordinate listed in Table 5B; v) at least 17, 18, 19, or 20 contiguous nucleotides of a sequence from (iv); or vi) a guide sequence that is at least 95%, 90%, or 85% identical to a sequence selected from (v). The composition may optionally further comprise any one of a nucleic acid (e.g., mRNA), polypeptide, composition, or lipid nucleic acid assembly composition disclosed herein.

In certain embodiments, the composition disclosed herein is used for altering a DNA sequence within the TRBC1 and/or TRBC2 gene in a cell. In certain embodiments, the composition disclosed herein is used for reducing the expression of the TRBC1 and/or TRBC2 gene in a cell. In some embodiments, the composition disclosed herein is used for immunotherapy of a subject.

J. Exemplary DNA Molecules, Vectors, Expression Constructs, Host Cells, and Production Methods

In certain embodiments, the disclosure provides a DNA molecule comprising a sequence encoding a polypeptide described herein. In some embodiments, the DNA molecule further comprises nucleic acids that do not encode the polypeptide. Nucleic acids that do not encode the polypeptide disclosed herein include, but are not limited to, promoters, enhancers, regulatory sequences, and nucleic acids encoding a gRNA.

In some embodiments, the DNA molecule further comprises a nucleotide sequence encoding a crRNA, a trRNA, or a crRNA and trRNA. In some embodiments, the nucleotide sequence encoding the crRNA, trRNA, or crRNA and trRNA comprises or consists of a guide sequence flanked by all or a portion of a repeat sequence from a naturally-occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA. In some embodiments, the crRNA and the trRNA are encoded by non-contiguous nucleic acids within one vector. In other embodiments, the crRNA and the trRNA may be encoded by a contiguous nucleic acid. In some embodiments, the crRNA and the trRNA are encoded by opposite strands of a single nucleic acid. In other embodiments, the crRNA and the trRNA are encoded by the same strand of a single nucleic acid.

In some embodiments, the DNA molecule further comprises a promoter operably linked to the sequence encoding any of the mRNAs encoding the polypeptide described herein. In some embodiments, the DNA molecule is an expression construct suitable for expression in a mammalian cell, e.g., a human cell or a mouse cell, such as a human hepatocyte or a rodent (e.g., mouse) hepatocyte. In some embodiments, the DNA molecule is an expression construct suitable for expression in a cell of a mammalian organ, e.g., a human liver or a rodent (e.g., mouse) liver. In some embodiments, the DNA molecule is a plasmid or an episome. In some embodiments, the DNA molecule is contained in a host cell, such as a bacterium or a cultured eukaryotic cell. Exemplary bacteria include proteobacteria such as E. coli. Exemplary cultured eukaryotic cells include primary hepatocytes, including hepatocytes of rodent (e.g., mouse) or human origin; hepatocyte cell lines, including hepatocytes of rodent (e.g., mouse) or human origin; human cell lines; rodent (e.g., mouse) cell lines; CHO cells; microbial fungi, such as fission or budding yeasts, e.g., Saccharomyces, such as S. cerevisiae; and insect cells.

In some embodiments, a method of producing an mRNA disclosed herein is provided. In some embodiments, such a method comprises contacting a DNA molecule described herein with an RNA polymerase under conditions permissive for transcription. In some embodiments, the contacting is performed in vitro, e.g., in a cell-free system. In some embodiments, the RNA polymerase is an RNA polymerase of bacteriophage origin, such as T7 RNA polymerase. In some embodiments, NTPs are provided that include at least one modified nucleotide as discussed above. In some embodiments, the NTPs include at least one modified nucleotide as discussed above and do not comprise UTP.

In some embodiments, an mRNA disclosed herein alone or together with one or more gRNAs, may be comprised within or delivered by a vector system of one or more vectors. In some embodiments, one or more of the vectors, or all of the vectors, may be DNA vectors. In some embodiments, one or more of the vectors, or all of the vectors, may be RNA vectors. In some embodiments, one or more of the vectors, or all of the vectors, may be circular. In other embodiments, one or more of the vectors, or all of the vectors, may be linear. In some embodiments, one or more of the vectors, or all of the vectors, may be enclosed in a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

Non-limiting exemplary viral vectors include adeno-associated virus (AAV) vector, lentivirus vectors, adenovirus vectors, helper dependent adenoviral vectors (HDAd), herpes simplex virus (HSV-1) vectors, bacteriophage T4, baculovirus vectors, and retrovirus vectors. In some embodiments, the viral vector may be an AAV vector. In other embodiments, the viral vector may a lentivirus vector. In some embodiments, the lentivirus may be non-integrating. In some embodiments, the viral vector may be an adenovirus vector. In some embodiments, the adenovirus may be a high-cloning capacity or “gutless” adenovirus, where all coding viral regions apart from the 5′ and 3′ inverted terminal repeats (ITRs) and the packaging signal (T) are deleted from the virus to increase its packaging capacity. In yet other embodiments, the viral vector may be an HSV-1 vector. In some embodiments, the HSV-1-based vector is helper dependent, and in other embodiments it is helper independent. For example, an amplicon vector that retains only the packaging sequence requires a helper virus with structural components for packaging, while a 30 kb-deleted HSV-1 vector that removes non-essential viral functions does not require helper virus. In additional embodiments, the viral vector may be bacteriophage T4. In some embodiments, the bacteriophage T4 may be able to package any linear or circular DNA or RNA molecules when the head of the virus is emptied. In further embodiments, the viral vector may be a baculovirus vector. In yet further embodiments, the viral vector may be a retrovirus vector. In embodiments using AAV or lentiviral vectors, which have smaller cloning capacity, it may be necessary to use more than one vector to deliver all the components of a vector system as disclosed herein. For example, one AAV vector may contain sequences encoding a Cas protein, while a second AAV vector may contain one or more guide sequences.

In some embodiments, the vector may be capable of driving expression of one or more coding sequences, such as the coding sequence of an mRNA disclosed herein, in a cell. In some embodiments, the cell may be a prokaryotic cell, such as, e.g., a bacterial cell. In some embodiments, the cell may be a eukaryotic cell, such as, e.g., a yeast, plant, insect, or mammalian cell. In some embodiments, the eukaryotic cell may be a mammalian cell. In some embodiments, the eukaryotic cell may be a rodent cell. In some embodiments, the eukaryotic cell may be a human cell. Suitable promoters to drive expression in different types of cells are known in the art. In some embodiments, the promoter may be wild type. In other embodiments, the promoter may be modified for more efficient or efficacious expression. In yet other embodiments, the promoter may be truncated yet retain its function. For example, the promoter may have a normal size or a reduced size that is suitable for proper packaging of the vector into a virus.

In some embodiments, the vector system may comprise one copy of a nucleotide sequence encoding a polypeptide disclosed herein. In other embodiments, the vector system may comprise more than one copy of a nucleotide sequence encoding a polypeptide disclosed herein. In some embodiments, the nucleotide sequence encoding a polypeptide disclosed herein may be operably linked to at least one transcriptional or translational control sequence. In some embodiments, the nucleotide sequence encoding the protein may be operably linked to at least one promoter.

In some embodiments, the promoter may be constitutive, inducible, or tissue-specific. In some embodiments, the promoter may be a constitutive promoter. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EF1a) promoter, ubiquitin promoters, actin promoters, tubulin promoters, immunoglobulin promoters, a functional fragment thereof, or a combination of any of the foregoing. In some embodiments, the promoter may be a CMV promoter. In some embodiments, the promoter may be a truncated CMV promoter. In other embodiments, the promoter may be an EF1a promoter. In some embodiments, the promoter may be an inducible promoter. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

In some embodiments, the promoter may be a tissue-specific promoter, e.g., a promoter specific for expression in the liver.

The vector may further comprise a nucleotide sequence encoding at least one gRNA. In some embodiments, the vector comprises one copy of the gRNA. In other embodiments, the vector comprises more than one copy of the gRNA. In embodiments with more than one gRNA, the gRNAs may be non-identical such that they target different target sequences, or may be identical in that they target the same target sequence. In some embodiments where the vectors comprise more than one gRNA, each gRNA may have other different properties, such as activity or stability within a complex with the polypeptide comprising a cytidine deaminase (e.g., an APOBEC3A deaminase (A3A)) and an RNA-guided nickase disclosed herein. In some embodiments, the nucleotide sequence encoding the gRNA may be operably linked to at least one transcriptional or translational control sequence, such as a promoter, a 3′ UTR, or a 5′ UTR. In one embodiment, the promoter may be a tRNA promoter, e.g., tRNALys3, or a tRNA chimera. See Mefferd et al., RNA. 2015 21:1683-9; Scherer et al., Nucleic Acids Res. 2007 35: 2620-2628. In some embodiments, the promoter may be recognized by RNA polymerase III (Pol III). Non-limiting examples of Pol III promoters include U6 and H1 promoters. In some embodiments, the nucleotide sequence encoding the gRNA may be operably linked to a mouse or human U6 promoter. In other embodiments, the nucleotide sequence encoding the gRNA may be operably linked to a mouse or human H1 promoter. In embodiments with more than one gRNA, the promoters used to drive expression may be the same or different. In some embodiments, the nucleotide encoding the crRNA of the gRNA and the nucleotide encoding the trRNA of the gRNA may be provided on the same vector. In some embodiments, the nucleotide encoding the crRNA and the nucleotide encoding the trRNA may be driven by the same promoter. In some embodiments, the crRNA and trRNA may be transcribed into a single transcript. For example, the crRNA and trRNA may be processed from the single transcript to form a double-molecule gRNA. Alternatively, the crRNA and trRNA may be transcribed into a single-molecule gRNA. In other embodiments, the crRNA and the trRNA may be driven by their corresponding promoters on the same vector. In yet other embodiments, the crRNA and the trRNA may be encoded by different vectors.

In some embodiments, the compositions comprise a vector system, wherein the system comprises more than one vector. In some embodiments, the vector system may comprise one single vector. In other embodiments, the vector system may comprise two vectors. In additional embodiments, the vector system may comprise three vectors. When different gRNAs are used for multiplexing, or when multiple copies of the gRNA are used, the vector system may comprise more than three vectors.

In some embodiments, the vector system may comprise inducible promoters to start expression only after it is delivered to a target cell. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

In additional embodiments, the vector system may comprise tissue-specific promoters to start expression only after it is delivered into a specific tissue.

In some embodiments, the vector may be delivered systemically. In some embodiments, the vector may be delivered into the hepatic circulation.

Lengthy table referenced here
US20240002820A1-20240104-T00001
Please refer to the end of the specification for access instructions.

### EXAMPLES

The following examples are provided to illustrate certain disclosed embodiments and are not to be construed as limiting the scope of this disclosure in any way.

As used in the Examples below, the term “editor” refers to an agent comprising a polypeptide that is capable of deaminating a base within a DNA molecule and it is a base editor. The editor may be capable of deaminating a cytidine (C) in DNA. The editor may include an RNA-guided nickase (e.g., Cas9 nickase) fused to a cytidine deaminase (e.g., an APOBEC3A deaminase (A3A)) by an optional linker. In some cases, the editor includes a UGI. In some embodiments, the editor lacks a UGI.

An exemplary editor used in the Examples below is BC22n (SEQ ID NO:3) which consists of a H. sapiens APOBEC3A fused to S. pyogenes-D10A SpyCas9 nickase by an XTEN linker, and mRNA encoding BC22n. An mRNA encoding BC22n (SEQ ID NO: 1) is also used.

### Example 1. General Methods

### 1.1. Preparation of Lipid Nanoparticles

In general, the lipid components were dissolved in 100% ethanol at various molar ratios. The RNA cargos (e.g., Cas9 mRNA and sgRNA) were dissolved in 25 mM citrate buffer, 100 mM NaCl, pH 5.0, resulting in a concentration of RNA cargo of approximately 0.45 mg/mL.

Unless otherwise specified, the lipid nucleic acid assemblies contained ionizable Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), cholesterol, DSPC, and PEG2k-DMG in a 50:38:9:3 molar ratio, respectively. The lipid nucleic acid assemblies were formulated with a lipid amine to RNA phosphate (N:P) molar ratio of about 6, and a ratio of gRNA to mRNA of 1:1 by weight, unless otherwise specified.

LNPs were prepared using a cross-flow technique utilizing impinging jet mixing of the lipid in ethanol with two volumes of RNA solutions and one volume of water. The lipids in ethanol were mixed through a mixing cross with the two volumes of RNA solution. A fourth stream of water was mixed with the outlet stream of the cross through an inline tee (See WO2016010840 FIG. 2). The LNPs were held for 1 hour at room temperature, and further diluted with water (approximately 1:1 v/v). LNPs were concentrated using tangential flow filtration on a flat sheet cartridge (Sartorius, 100 kD MWCO) and buffer exchanged using PD-10 desalting columns (GE) into 50 mM Tris, 45 mM NaCl, 5% (w/v) sucrose, pH 7.5 (TSS). Alternatively, the LNP's were optionally concentrated using 100 kDa Amicon spin filter and buffer exchanged using PD-10 desalting columns (GE) into TSS. The resulting mixture was then filtered using a 0.2 μm sterile filter. The final LNP was stored at 4° C. or −80° C. until further use.

1.2. In Vitro Transcription (“IVT”) of mRNA

Capped and polyadenylated mRNA containing N1-methyl pseudo-U was generated by in vitro transcription using a linearized plasmid DNA template and T7 RNA polymerase. Plasmid DNA containing a T7 promoter, a sequence for transcription, and a polyadenylation region was linearized by incubating at 37° C. for 2 hours with XbaI with the following conditions: 200 ng/μL plasmid, 2 U/μL XbaI (NEB), and 1× reaction buffer. The XbaI was inactivated by heating the reaction at 65° C. for 20 min. The linearized plasmid was purified from enzyme and buffer salts. The IVT reaction to generate modified mRNA was performed by incubating at 37° C. for 1.5-4 hours in the following conditions: 50 ng/μL linearized plasmid; 2-5 mM each of GTP, ATP, CTP, and N1-methyl pseudo-UTP (Trilink); 10-25 mM ARCA (Trilink); 5 U/μL T7 RNA polymerase (NEB); 1 U/μL Murine RNase inhibitor (NEB); 0.004 U/μL Inorganic E. coli pyrophosphatase (NEB); and 1× reaction buffer. TURBO DNase (ThermoFisher) was added to a final concentration of 0.01 U/μL, and the reaction was incubated for an additional 30 minutes to remove the DNA template. The mRNA was purified using a MegaClear Transcription Clean-up kit (ThermoFisher) or a RNeasy Maxi kit (Qiagen) per the manufacturers' protocols. Alternatively, the mRNA was purified through a precipitation protocol, which in some cases was followed by HPLC-based purification. Briefly, after the DNase digestion, mRNA is purified using LiCl precipitation, ammonium acetate precipitation and sodium acetate precipitation. For HPLC purified mRNA, after the LiCl precipitation and reconstitution, the mRNA was purified by RP-IP HPLC (see, e.g., Kariko, et al. Nucleic Acids Research, 2011, Vol. 39, No. 21 e142). The fractions chosen for pooling were combined and desalted by sodium acetate/ethanol precipitation as described above. In a further alternative method, mRNA was purified with a LiCl precipitation method followed by further purification by tangential flow filtration. RNA concentrations were determined by measuring the light absorbance at 260 nm (Nanodrop), and transcripts were analyzed by capillary electrophoresis by Bioanlayzer (Agilent).

Nme2Cas9 mRNA was generated from plasmid DNA encoding an open reading frame according to SEQ ID NOs: 360 (see sequences in Table 5C). Streptococcus pyogenes (“Spy”) Cas9 mRNA was generated from plasmid DNA encoding an open reading frame according to SEQ ID NOs: 8, 11, or 23 (see sequences in Table 5C). BC22n mRNA was generated from plasmid DNA encoding an open reading frame according to SEQ ID NOs: 2 or 5. BC22 mRNA was generated from plasmid DNA encoding an open reading frame according to SEQ ID NO: 20. BC22 with 2×UGI mRNA was generated from plasmid DNA encoding an open reading frame according to SEQ ID No: 29. UGI mRNA was generated from plasmid DNA encoding an open reading frame according to SEQ ID NOs: 26 or 35. BE3 mRNA was generated from plasmid DNA encoding an open reading frame according to SEQ ID NO: 14 or 17. BE4Max mRNA was generated from plasmid DNA encoding an open reading frame according to SEQ ID NO: 32. When the sequences cited in this paragraph are referred to below with respect to RNAs, it is understood that Ts should be replaced with Us (which were N1-methyl pseudouridines as described above). Messenger RNAs used in the Examples include a 5′ cap and a 3′ polyadenylation region, e.g., up to 100 nts.

### 1.3. Next-Generation Sequencing (“NGS”) and Analysis for On-Target Editing Efficiency

Genomic DNA was extracted using QuickExtract™ DNA Extraction Solution (Lucigen, Cat. QE09050) according to the manufacturer's protocol. To quantitatively determine the efficiency of editing at the target location in the genome, deep sequencing was utilized to identify the presence of insertions and deletions introduced by gene editing. PCR primers were designed around the target site within the gene of interest (e.g., TRAC) and the genomic area of interest was amplified. Primer sequence design was done as is standard in the field.

Additional PCR was performed according to the manufacturer's protocols (Illumina) to add chemistry for sequencing. The amplicons were sequenced on an Illumina MiSeq instrument. The reads were aligned to the human reference genome (e.g., hg38) after eliminating those having low quality scores. Reads that overlapped the target region of interest were re-aligned to the local genome sequence to improve the alignment. Then the number of wild type reads versus the number of reads which contain C-to-T mutations, C-to-A/G mutations or indels was calculated. Insertions and deletions were scored in a 20 bp region centered on the predicted Cas9 cleavage site. Indel percentage is defined as the total number of sequencing reads with one or more base inserted or deleted within the 20 bp scoring region divided by the total number of sequencing reads, including wild type. C-to-T mutations or C-to-A/G mutations were scored in a 40 bp region including 10 bp upstream and 10 bp downstream of the 20 bp sgRNA target sequence. The C-to-T editing percentage is defined as the total number of sequencing reads with either one or more C-to-T mutations within the 40 bp region divided by the total number of sequencing reads, including wild type. The percentage of C-to-A/G mutations are calculated similarly.

### Example 1A—C-to-T Conversion

APOBEC3A deaminase-Cas9D10A editor was evaluated for efficiency of C-to-T conversion activity and the span of the C-to-T conversion window. C-to-T conversion activity and window results were compared against construct BC27 encoding BE3 (Komor A C, Kim Y B, Packer M S, Zuris J A, Liu D R. Programmable editing of a target base in genomic DNA without double-stranded DNA cleavage. Nature. 2016; 533(7603):420-424.) Constructs were each evaluated in triplicate against 5 sgRNA in a single experiment.

Plasmid A, using a pUC19 backbone (GenBank® Accession Number U47119), expresses an S. pyogenes single-guide RNA (sgRNA) from a U6 promoter. Plasmid B, called pCI, expresses base editor constructs from a CMV promoter which are comprised of the candidate deaminase fused to S. pyogenes-D10A-Cas9 by an XTEN linker which is subsequently fused to one copy of UGI and one copy of SV40 NLS. U-2OS cells growing in Dulbecco's Modified Eagle's Medium (DMEM) supplemented with 10% Fetal Bovine Serum (FBS) in 96-well plates were transfected using Mirus TransIT-X2® with 100 ng each of plasmid A and plasmid B. Cells were washed and resuspended in fresh media 24 hours after initial transfection. After an additional 48 hours, the media was removed and the cells were lysed with QuickExtract™ DNA Extraction Solution (Lucigen, Cat. QE09050).

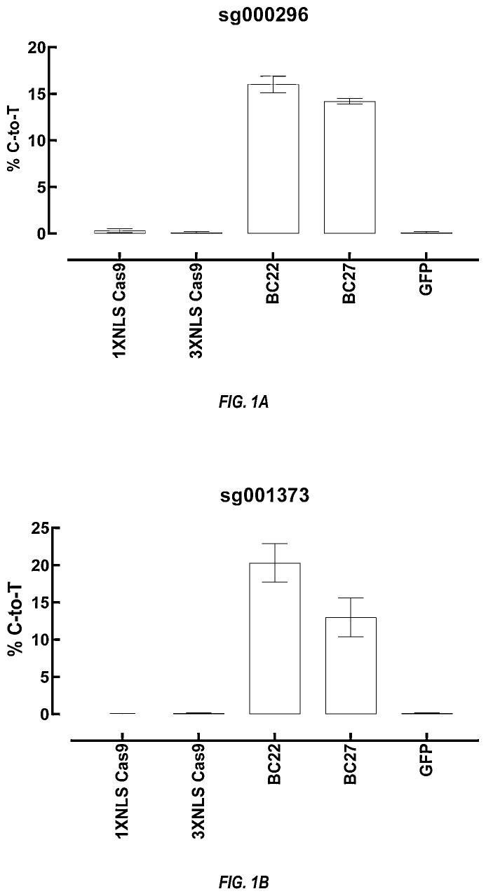

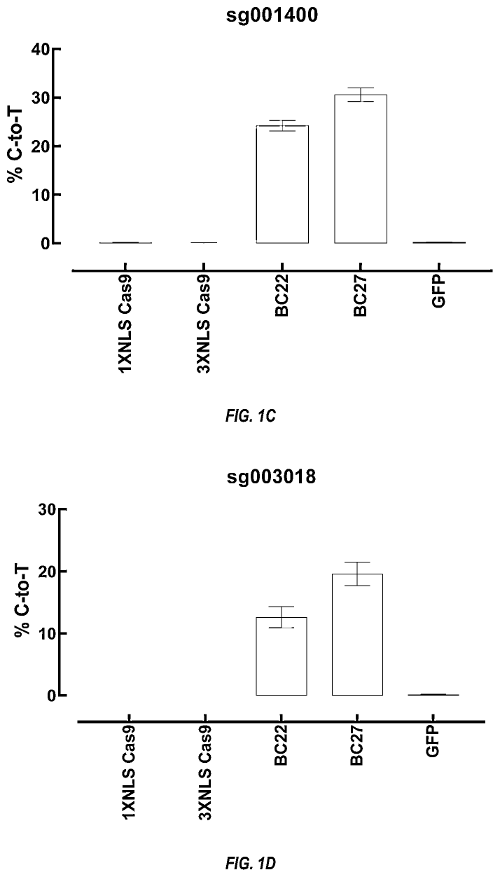

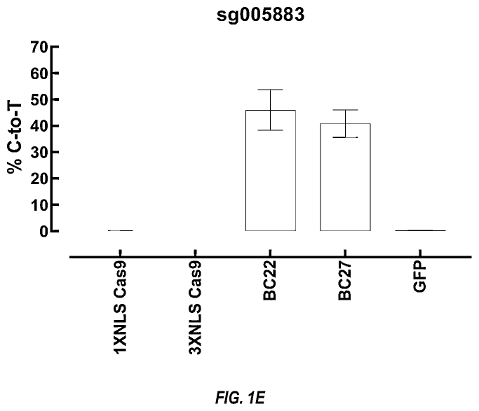

Construct BC22 encoding the H. sapiens APOBEC3A deaminase (Uniprot ID P31941) in the fusion had significantly greater C-to-T conversion activity than BC27 encoding the rat APOBEC1 deaminase (Uniprot ID P38483) with more than one guide (sg000296: P value 0.0303; sg001373, P value 0.0263). The average activity of BC22 was comparable to that of BC27 across all guides (Table 6, FIGS. 1A-IE).

Across all 5 sgRNAs tested, BC22 was significantly more likely to convert all target cytosines to thymidines than BC27 (Table 7, FIGS. 2A-2E, P value<0.005). A target cytosine is any cytosine within positions 1-10 of the protospacer target sequence (underlined) or in first position 5′ of the target sequence The guide sequence for sg000296 is CCUUCCGAAAGAGGCCCCCC (SEQ ID NO: 156), and the locus has 4 target cytosines. The target guide sequence for sg001373 is UC̅C̅C̅UGGCUGAGGAUCCCCA (SEQ ID NO: 157), and the locus has 5 target cytosines, including the first position 5′ of the target sequence. The target guide sequence for sg001400 is ACUCACGAUGAAAUCCUGGA (SEQ ID NO: 158), and the locus has 4 target cytosines including the first position 5′ of the target sequence. The target guide sequence for sg003018 is GAGCCCCCCACUGUGGUGAC (SEQ ID NO: 160), and the locus has 6 target cytosines. The guide sequence for sg005883 is CCCCCCGCCGUGUUUGUGGG (SEQ ID NO: 159), and the locus has 8 target cytosines.

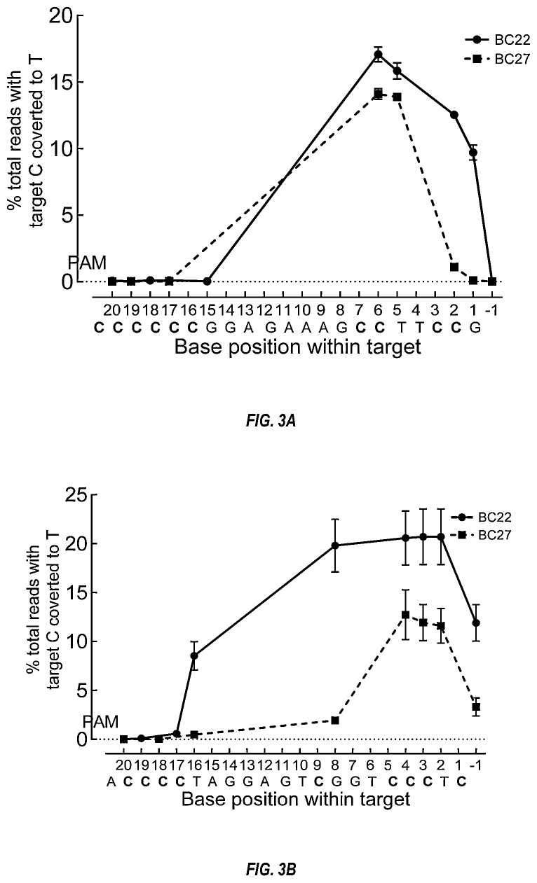

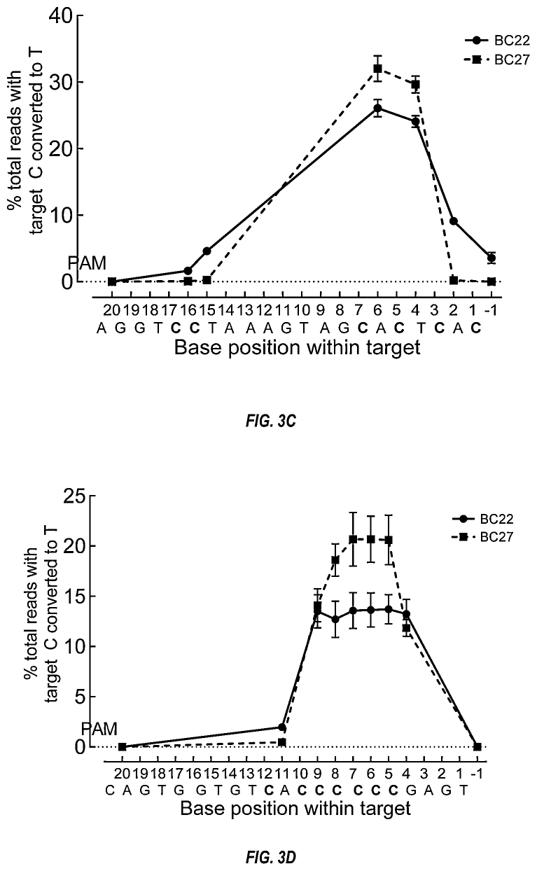

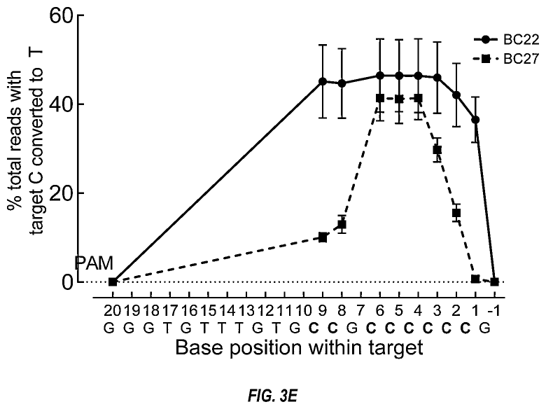

BC22 converted a significantly larger proportion and positional range of target cytosine than BC27 (FIGS. 3A-3E). converted a significantly larger proportion and positional range of target cytosines than BC27 (FIGS. 3A-3E). This wider C-to-T conversion window held true even for guides that BC22 had less total C-to-T conversion activity on such as sg001400, sg003018 (Table 7, Figure FIGS. 3C-3E).

TABLE 6
Percent of total reads containing at least 1 cytosine to
thymidine conversion. (n = 3)

| | sg000296 | | sg0001373 | | sg001400 | | sg003018 | | sg005883 | |
|---|---|---|---|---|---|---|---|---|---|---|
| Construct | Mean | SD | Mean | SD | Mean | SD | Mean | SD | Mean | SD |
| 1XNLS | 0.3 | 0.2 | 0.0 | 0.1 | 0.1 | 0.1 | 0.0 | 0.0 | 0.0 | 0.1 |
| Cas9 | | | | | | | | | | |
| 3XNLS | 0.1 | 0.1 | 0.1 | 0.1 | 0.0 | 0.1 | 0.0 | 0.0 | 0.0 | 0.0 |
| Cas9 | | | | | | | | | | |
| BC22 | 16.0 | 0.9 | 20.3 | 2.6 | 24.2 | 1.1 | 12.6 | 1.7 | 46.0 | 7.7 |
| BC27 | 14.2 | 0.3 | 13.0 | 2.6 | 30.6 | 1.4 | 19.6 | 1.9 | 40.8 | 5.2 |
| GFP | 0.1 | 0.1 | 0.1 | 0.1 | 0.2 | 0.1 | 0.1 | 0.1 | 0.2 | 0.1 |

TABLE 7
Percent of C-to-T edited reads in which all target cytosines
have been converted to thymidines (sg000296, sg001373,
sg001400, sg003018). For sg005883, percent of edited reads in which
at least 6 of 8 target cytosines have been converted to thymidines.

| | sg000296 | | sg001373 | | sg001400 | | sg003018 | | sg005883 | |
|---|---|---|---|---|---|---|---|---|---|---|
| Construct | Mean | SD | Mean | SD | Mean | SD | Mean | SD | Mean | SD |
| 1XNLS | 0 | 0 | n/d | n/d | 0 | 0 | 0 | 0 | 9.4 | 9.5 |
| Cas9 | | | | | | | | | | |
| 3XNLS | 0 | 0 | 0 | 0 | 0 | 0 | 0 | 0 | 7.0 | 3.9 |
| Cas9 | | | | | | | | | | |
| BC22 | 45.1 | 2.1 | 34.6 | 5.5 | 11.1 | 0.9 | 80.2 | 1.4 | 61.8 | 10.1 |
| BC27 | 0.6 | 0 | 3.3 | 0.3 | 0.1 | 0.2 | 43.2 | 6.7 | 15.2 | 4.8 |
| GFP | 0 | 0 | n/d | n/d | 0 | 0 | 0 | 0 | 0 | 0 |

(n/d = not done)

### Example 2—Effects of Base Excision Repair Genes on Editing

The effects of additional UGI gene expression in trans on C-to-T conversion activity relative to unwanted base excision repair activity were investigated in various cell lines.

U-2OS and HuH-7 cells grown in Dulbecco's Modified Eagle's Medium (DMEM) supplemented with 10% FBS in 96-well plates were co-transfected using Mirus TransIT-X2® with 100 ng of BC22 or BC27, 100 ng of another CMV-driven overexpression plasmid (pcDNA3.1) or a control plasmid (pMAX) and 6.25 pmol of single-guide G000297. The ORFs tested in tandem with BC22 and BC27 were green fluorescent protein (pMAX-GFP, negative control), Uracil DNA glycosylase (pCDNA3.1-UNG), Single-Strand-Selective Monofunctional Uracil-DNA Glycosylase 1 (pcDNA3.1-SMUG1) and Uracil Glycosylase Inhibitor (pCDNA3.1-UGI). UNG and SMUG1 are base excision repair proteins that remove uracil from DNA. UGI is the small protein that binds and inhibits UDG. It has been reported that when UGI is fused to the constructs it increases the rate of C-to-T mutations relative to the other outcomes of C-to-A/G mutations and indels (Liu et al, Nature 2016). To determine whether the addition of UNG transcript knockdown further enriched for C-to-T-only edits, an additional transfection condition included either BC22 or BC27, pcDNA3.1-UGI, G000297 and a pool of siRNA targeting UNG (Dharmacon, #M-011795-00). Three days after transfection, media was removed and the cells were lysed with QuickExtract™ DNA Extraction Solution (Lucigen, Cat. QE09050).

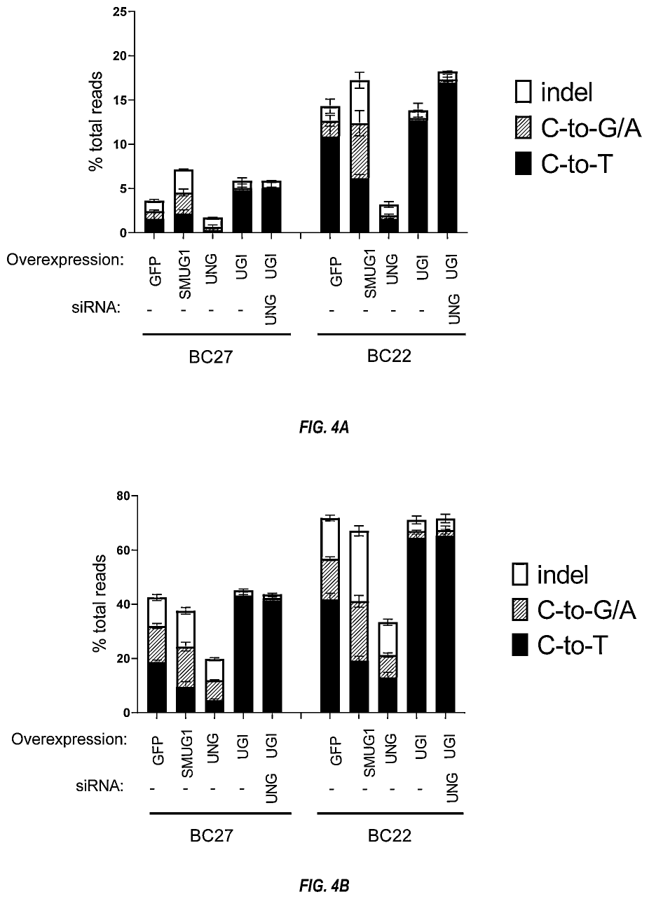

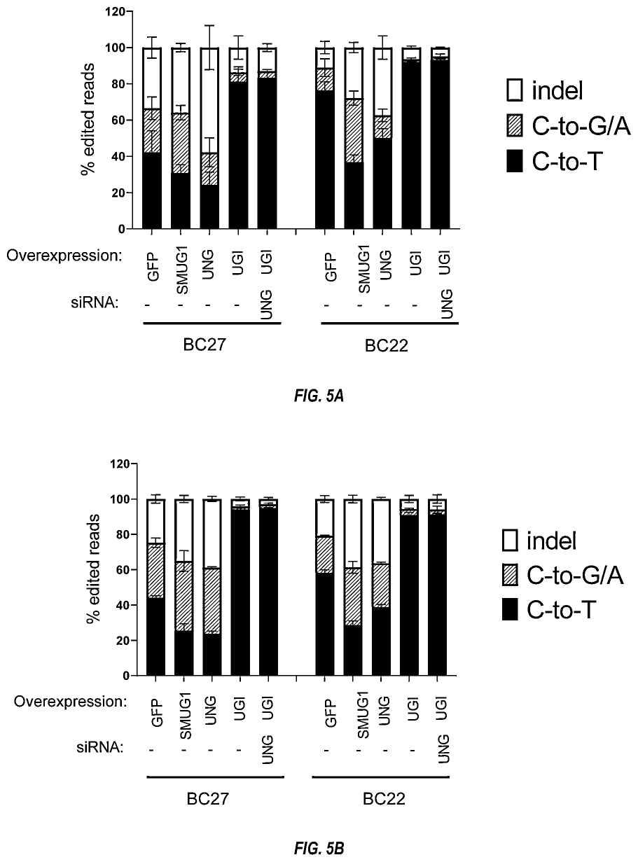

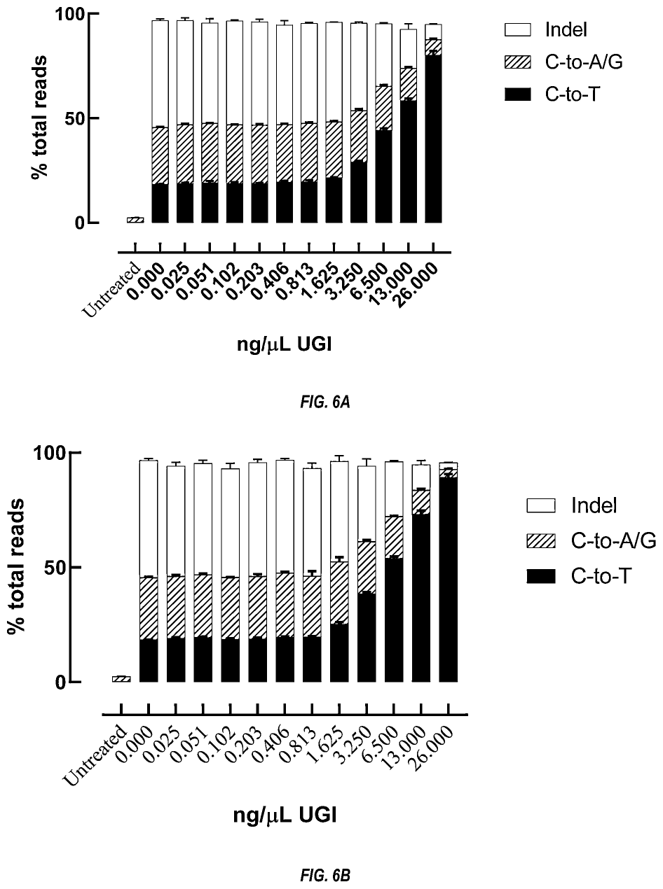

In HuH-7 cells, overexpressing base excision repair proteins UNG or SMUG1 with either BC22 or BC27 led to a 1.3-1.7-fold significant increase in the proportion of C-to-A/G mutations and indels relative to C-to-T only mutations when compared to overexpression of the negative control GFP (Table 11, FIG. 5B). Conversely, blocking UNG with UGI overexpression led to a 4.5-10.8-fold decrease in reads containing C-to-A/G mutations and indels with a significant 1.56-2.2-fold increase in C-to-T-only mutations (Table 11, FIG. 5B). Additional UGI overexpression led to, on average, 94% of total edits being C-to-T-only edits whether using BC22 or BC27 (FIG. 4B). Similar trends were seen with U-2OS (Table 8, Figure FIGS. 4A, 5A) with the one outstanding difference that SMUG1 and UNG overexpression did not lead to significant changes in relative proportions of editing outcomes. In both cell lines the addition of siRNAs targeting UNG gene to the UGI overexpression plasmid did not increase the proportion of C-to-T-only edits over UGI overexpression alone (Table 8). Finally, in both cell lines BC22 total C-to-T conversion activity was higher than BC27 (FIGS. 4A, 4B), suggesting BC22 has higher innate C-to-T conversion activity than BC27.

TABLE 8
Editing as a % of total reads in U-2OS cells (n = 3)

| U-2OS | | | | | | | |
|---|---|---|---|---|---|---|---|
| Con- | Overexpression/ | C-to-T % | | C-to-A/G % | | Indel % | |
| struct | SIRNA | Mean | SD | Mean | SD | Mean | SD |
| BC27 | GFP+ | 1.57 | 0.99 | 0.86 | 0.14 | 1.18 | 0.16 |
| | SMUG1+ | 2.13 | 0.45 | 2.4 | 0.39 | 2.59 | 0.09 |
| | UDG+ | 0.3 | 0.26 | 0.36 | 0.22 | 1.05 | 0.04 |
| | UGI+ | 4.77 | 0.75 | 0.29 | 0.08 | 0.8 | 0.34 |
| | siUDG, UGI+ | 4.9 | 0.75 | 0.22 | 0.05 | 0.76 | 0.03 |
| BC22 | GFP+ | 10.87 | 1.58 | 1.78 | 0.61 | 1.64 | 0.8 |
| | SMUG1+ | 6.17 | 0.4 | 6.21 | 1.42 | 4.85 | 0.9 |
| | UDG+ | 1.57 | 0.06 | 0.4 | 0.13 | 1.21 | 0.32 |
| | UGI+ | 12.7 | 1.93 | 0.24 | 0.13 | 0.86 | 0.05 |
| | siUDG, UGI+ | 16.97 | 0.93 | 0.36 | 0.25 | 0.9 | 0.04 |

TABLE 9
Fold change relative to GFP in % of edited reads in U-2OS cells

| | Overexpression/ | | | C-to-A/G | |
|---|---|---|---|---|---|
| Construct | siRNA | C-to-T | P value | or Indel | P value |
| BC27 | GFP+ | not applicable | | | |
| | SMUG1+ | −1.37 | 0.1953 | 1.20 | 0.1953 |
| | UDG+ | −1.75 | 0.0864 | 1.31 | 0.0864 |
| | UGI+ | 1.92 | 0.0091 | −3.09 | 0.0091 |
| | siUDG, UGI+ | 1.97 | 0.0042 | −3.48 | 0.0042 |
| BC22 | GFP+ | not applicable | | | |
| | SMUG1+ | −2.08 | 0.0004 | 2.67 | 0.0004 |
| | UDG+ | −1.52 | 0.0031 | 2.10 | 0.0031 |
| | UGI+ | 1.21 | 0.0048 | −2.97 | 0.0048 |
| | siUDG, UGI+ | 1.22 | 0.0041 | −3.46 | 0.0041 |

TABLE 10
Percent editing under different DNA repair pathway
conditions in HuH-7 cells

| HuH-7 | | | | | | | |
|---|---|---|---|---|---|---|---|
| | Over- | | | | | | |
| Con- | expression/ | C-to-T % | | C-to-A/G % | | Indel % | |
| struct | SIRNA | Mean | SD | Mean | SD | Mean | SD |
| BC27 | GFP+ | 18.70 | 0.70 | 13.24 | 0.95 | 10.55 | 1.11 |
| | SMUG1+ | 9.57 | 1.91 | 14.78 | 1.64 | 13.23 | 1.24 |
| | UDG+ | 4.57 | 0.46 | 7.45 | 0.23 | 7.74 | 0.49 |
| | UGI+ | 42.40 | 2.10 | 0.82 | 0.34 | 1.88 | 0.47 |
| | SiUDG, UGI+ | 41.37 | 2.73 | 0.94 | 0.37 | 1.32 | 0.30 |
| BC22 | GFP+ | 41.77 | 2.30 | 15.02 | 0.73 | 15.00 | 1.06 |
| | SMUG1+ | 19.20 | 1.61 | 21.89 | 2.17 | 25.98 | 1.86 |
| | UDG+ | 12.97 | 1.89 | 8.27 | 0.82 | 12.11 | 1.14 |
| | UGI+ | 64.53 | 1.32 | 2.50 | 0.30 | 4.05 | 1.45 |
| | SiUDG, UGI+ | 65.33 | 2.28 | 2.05 | 1.45 | 4.24 | 1.53 |

TABLE 11
Fold change relative to GFP in % of edited reads in Huh-7 cells

| | Overexpression/ | | | C-to-A/G | |
|---|---|---|---|---|---|
| Construct | siRNA | C-to-T | P value | or indel | P value |
| BC27 | GFP+ | not applicable | | | |
| | SMUG1+ | −1.73 | 0.0014 | 1.33 | 0.001 |
| | UDG+ | −1.86 | <0.0001 | 1.37 | <0.0001 |
| | UGI+ | 2.13 | <0.0001 | −9.30 | <0.0001 |
| | siUDG, UGI+ | 2.15 | <0.0001 | −10.81 | <0.0001 |
| BC22 | GFP+ | not applicable | | | |
| | SMUG1+ | −2.03 | <0.0001 | 1.71 | <0.0001 |
| | UDG+ | −1.50 | 0.0001 | 1.46 | 0.0001 |
| | UGI+ | 1.56 | <0.0001 | −4.53 | <0.0001 |
| | siUDG, UGI+ | 1.57 | <0.0001 | −4.76 | <0.0001 |

### Example 3—UGI mRNA Titration in T Cells

The mRNA encodes a fusion protein, BC22n (SEQ ID NO: 3), which is, from N-terminus to C-terminus, a H. sapiens APOBEC3A, an XTEN linker, a D10ACas9 nickase, a linker, and an SV40 NLS. Notably the BC22n polypeptide lacks a UGI. T cells were edited with BC22n using and a variable amount of UGI mRNA to determine the impact of trans UGI levels on editing profile. This experiment was performed using 2 different UGI mRNAs each encoding the same protein using a different open reading frame (SEQ ID Nos: 26 and 35).

### 3.1 T Cell Preparation

Healthy human donor apheresis was obtained commercially (Hemacare), and cells were washed and re-suspended in CliniMACS® PBS/EDTA buffer (Miltenyi Biotec Cat. No. 130-070-525) on the LOVO device. T cells were isolated via positive selection using CD4 and CD8 magnetic beads (Miltenyi Biotec Cat. No. 130-030-401/130-030-801) using the CliniMACS® Plus and CliniMACS® LS disposable kit. T cells were aliquoted into vials and cryopreserved in a 1:1 formulation of Cryostor® CS10 (StemCell Technologies Cat. No. 07930) and Plasmalyte A (Baxter Cat. No. 2B2522X) for future use. Upon thaw, T cells were plated at a density of 1.0×10\hat{ }6 cells/mL in T cell basal media composed of X-VIVO 15™ serum-free hematopoietic cell medium (Lonza Bioscience) containing 5% (v/v) of fetal bovine serum, 50 μM of 2-Mercaptoethanol, 10 mM of N-Acetyl-L-(+)-cysteine, 10 U/mL of Penicillin-Streptomycin, in addition to 1× cytokines (200 U/mL of recombinant human interleukin-2, 5 μg/mL of recombinant human interleukin-7 and 5 μg/mL of recombinant human interleukin-15). T cells were activated with TransAct™ (1:100 dilution, Miltenyi Biotec). Cells were expanded in T cell basal media for 72 hours prior to electroporation.

3.2 Electroporation of T Cells Using mRNA and sgRNA

A solution containing mRNAs encoding BC22n (SEQ ID NO: 2) and one species of UGI (SEQ ID NO: 26 or 35) was prepared in sterile water. 50 μM TRAC targeting sgRNA G016017 (SEQ ID NO: 184) were removed from their storage plates and denatured for 2 minutes at 95° C. before cooling on ice. Seventy-two hours post activation, T cells were harvested, centrifuged, and resuspended at a concentration of 12.5×10\hat{ }6 T cells/mL in P3 electroporation buffer (Lonza). For each well to be electroporated, 1×10\hat{ }5 T cells were mixed with 200 ng of BC22n mRNA, 20 pmols of sgRNA and UGI mRNA ranging from 0.02 ng to 26 ng (dilution factor of 12.24), in a final volume of 20 μL of P3 electroporation buffer. This mRNA+sgRNA+T cell mix was transferred in triplicate to a 96-well Nucleofector™ plate and electroporated using the manufacturer's pulse code. Electroporated T cells were immediately rested in 80 μL of T cell basal media without cytokines for 10 minutes before being transferred to a new flat-bottom 96-well plate containing an additional 100 μL of T cell basal media with 2× concentration of cytokines. Electroporated T cells were subsequently cultured for 3 additional days and were collected for NGS sequencing as described in Example 1.

The constant dose of 200 ng of BC22n mRNA drove high total editing (>92%) across all conditions (Tables 12 and 13, sum of indel, C-to-A/G and C-to-T). The proportion of edits that were C-to-T-only increased in a dose-responsive manner to UGI mRNA concentration in the electroporation. At the highest dose of either UGI mRNAs SEQ ID NOs: 25 or 34, the percentage of sequencing reads containing indels and C-to-A/G mutations dropped to 7.4±0.4, 7.4±0.6 and 2.9±0.2, 3.6±0.3, respectively (Tables 12 and 13).

TABLE 12
Editing as percent of total reads with UGI
mRNA (SEQ ID NO: 26)

| | % Editing | | | | | |
|---|---|---|---|---|---|---|
| UGI mRNA | Indel | | C-to-A/G | | C-to-T | |
| (ng) | Mean | SD | Mean | SD | Mean | SD |
| Untreated | 0.2 | 0.1 | 2.1 | 0.0 | 0.2 | 0.0 |
| 0.000 | 51.2 | 0.8 | 27.2 | 0.4 | 18.3 | 0.3 |
| 0.025 | 50.0 | 1.1 | 28.2 | 0.6 | 18.6 | 0.6 |
| 0.051 | 48.2 | 2.0 | 28.4 | 0.3 | 19.0 | 1.0 |
| 0.102 | 49.8 | 0.5 | 28.1 | 0.3 | 18.7 | 0.8 |
| 0.203 | 49.5 | 1.1 | 27.9 | 0.6 | 18.7 | 0.3 |
| 0.406 | 47.7 | 2.0 | 27.5 | 0.5 | 19.4 | 0.7 |
| 0.813 | 47.8 | 0.6 | 28.0 | 0.5 | 19.5 | 0.9 |
| 1.625 | 47.9 | 0.1 | 26.6 | 0.5 | 21.5 | 0.3 |
| 3.250 | 41.9 | 0.6 | 24.7 | 0.8 | 28.9 | 0.7 |
| 6.500 | 30.0 | 0.5 | 21.1 | 0.6 | 44.1 | 1.0 |
| 13.000 | 18.7 | 2.6 | 15.5 | 0.6 | 58.3 | 1.0 |
| 26.000 | 7.4 | 0.4 | 7.4 | 0.6 | 80.1 | 2.1 |

TABLE 13
Editing as percent of total reads with UGI
mRNA (SEQ ID NO: 35)

| | % Editing | | | | | |
|---|---|---|---|---|---|---|
| UGI mRNA | Indel | | C-to-A/G | | C-to-T | |
| (ng) | Mean | SD | Mean | SD | Mean | SD |
| Untreated | 0.2 | 0.1 | 2.1 | 0.0 | 0.2 | 0.0 |
| 0.000 | 51.2 | 0.8 | 27.2 | 0.4 | 18.3 | 0.3 |
| 0.025 | 48.2 | 1.7 | 27.0 | 0.7 | 19.0 | 0.5 |
| 0.051 | 48.7 | 1.3 | 27.3 | 0.7 | 19.4 | 0.4 |
| 0.102 | 47.5 | 2.3 | 27.1 | 0.2 | 18.5 | 0.5 |
| 0.203 | 49.7 | 1.3 | 27.4 | 0.9 | 18.7 | 0.7 |
| 0.406 | 49.4 | 0.7 | 28.0 | 0.6 | 19.4 | 0.4 |
| 0.813 | 47.1 | 2.3 | 26.6 | 2.2 | 19.6 | 0.5 |
| 1.625 | 43.8 | 2.5 | 27.3 | 2.0 | 25.2 | 1.0 |
| 3.250 | 33.1 | 3.0 | 22.8 | 0.8 | 38.4 | 0.7 |
| 6.500 | 24.0 | 0.4 | 18.2 | 0.3 | 53.9 | 0.8 |
| 13.000 | 11.2 | 1.9 | 10.5 | 0.6 | 73.1 | 1.6 |
| 26.000 | 2.9 | 0.2 | 3.6 | 0.3 | 89.1 | 1.6 |

### Example 4: In Vivo Editing with UGI in Cis

LNPs formulated with editor mRNA and sgRNA G000282 at a 1:1 RNA weight ratio were tested for editing efficacy and editing outcomes in vivo. Experimental groups included TSS buffer only; mRNA encoding cleavase-spCas9 (SEQ ID NO: 8), mRNA encoding BC22 which includes human APOBEC3A fused to D10A SpyCas9 and one copy of UGI (SEQ ID NO: 20); and mRNAs encoding BE3 which includes rat APOBEC1 fused to D10A SpyCas9 and one copy of UGI (SEQ ID NOs: 14 and 17).

CD-1 female mice ranging from 6-10 weeks of age (n=5/group) were used in this study. LNPs were administered intravenously via tail vein injection at a dose of 1 mg/kg of total RNA to body weight. The animals were periodically observed for adverse effects for at least 24 hours post dose. Six days after treatment, animals were euthanized by cardiac puncture under isoflurane anesthesia; blood and liver tissue were collected for downstream analysis. Blood was collected into serum separator tubes or into tubes containing buffered sodium citrate for plasma. Liver punches weighting between 5 and 15 mg were collected for isolation of genomic DNA and total RNA.

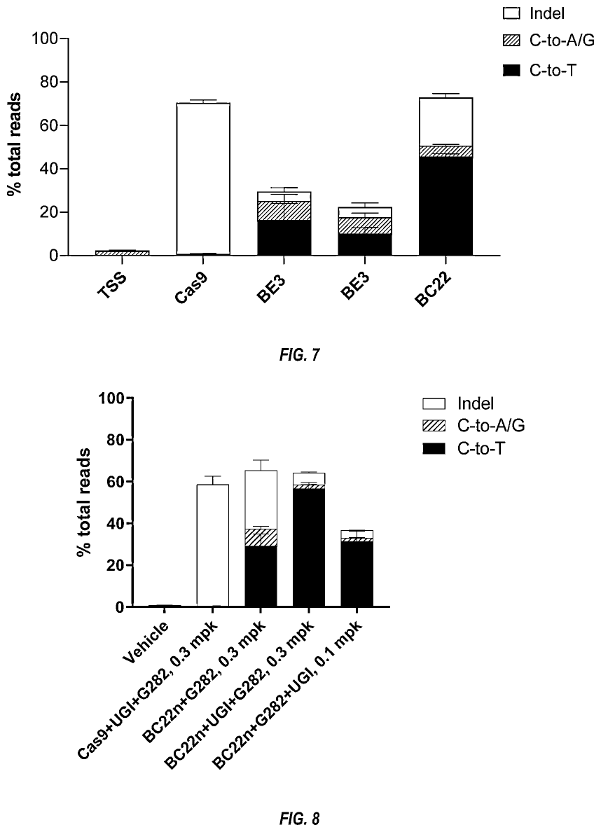

Table 14 and FIG. 7A describe the editing results in mouse liver. The construct containing human APOBEC3A (BC22) showed total editing levels comparable to those of the Cas9 cleavase construct, while the rat APOBEC1 (BE3) constructs showed less than half that total editing activity. The construct containing human APOBEC3A also achieved more than twice the absolute percentage of C-to-T base conversion compared to the rat APOBEC1 constructs (BE3).

TABLE 14
TTR editing in liver tissue

| | % Editing | | | | | |
|---|---|---|---|---|---|---|
| | C-to- T | | C-to-A/G | | Indel | |
| Editor | Mean | SD | Mean | SD | Mean | SD |
| TSS | 0.15 | 0.04 | 1.95 | 0.26 | 0.31 | 0.11 |
| Cas9 (SEQ | 0.05 | 0.02 | 0.72 | 0.23 | 69.59 | 1.28 |
| ID NO: 8) | | | | | | |
| BE3 (SEQ ID | 16.53 | 7.49 | 8.69 | 3.01 | 4.31 | 1.79 |
| NO: 14) | | | | | | |
| BE3 (SEQ ID | 10.23 | 2.72 | 7.41 | 1.90 | 4.71 | 1.92 |
| NO: 17) | | | | | | |
| BC22 (SEQ | 45.75 | 1.17 | 4.98 | 0.49 | 22.0 | 1.82 |
| ID NO: 20) | | | | | | |

(Indel = insertions or deletions;
SD = standard deviation)

### Example 5—In Vivo Editing with UGI in Trans

In vivo editing profiles of deaminase containing constructs were compared to Cas9 when UGI was delivered in trans (as a separate mRNA). The constructs used encoded a fusion protein including D10A SpyCas9 with a deaminase. Further, gene expression differences between these editing conditions were analyzed through transcriptomic analysis.

### 5.1 In Vivo Editing as Assayed by NGS

Forty-five commercially available CD-1 female mice ranging from 6-10 weeks of age (n=5 per group) were used in this study. Animals were weighed pre-dose for dosing calculations. Each RNA species was formulated separately in an LNP. Formulations containing editor mRNA, UGI mRNA and sgRNA were mixed in a w/w ratio of RNA cargos of 6:3:2 (editor mRNA:sgRNA:UGI mRNA). The formulation mixture for Group 3 contained only editor mRNA and sgRNA and these were mixed in a w/w ratio of 2:1 (editor mRNA:sgRNA). Apart from the negative control group, which was dosed with TSS buffer only, all groups received sgRNA G000282. Formulations were administered intravenously via tail vein injection according to the doses listed in Table 15. Animals were periodically observed for adverse effects for at least 24 hours post-dose. Six days after treatment, animals were euthanized by cardiac puncture under isoflurane anesthesia; liver tissue were collected for downstream analysis. Liver punches weighing between 5 and 15 mg were collected for isolation of genomic DNA and total RNA. Genomic DNA samples were analyzed with NGS sequencing as described in Example 1.

TABLE 15
Animal groups and the respective LNPs.

| | | Editor mRNA + |
|---|---|---|
| Sample | LNP cargos | sgRNA dose (mg/kg) |
| TSS | None | 0 |
| Cas9, UGI | Cas9 (SEQ ID NO: 11) | 0.3 mg/kg |
| | UGI (SEQ ID NO: 26) | |
| | G0000282 | |
| BC22n | BC22n (SEQ ID NO: 2) | 0.3 mg/kg |
| | G0000282 | |
| BC22n, UGI | BC22n (SEQ ID NO: 2) | 0.3 mg/kg |
| | UGI (SEQ ID NO: 26) | Or |
| | G0000282 | 0.1 mg/kg |

Editing data are shown in Table 16 and FIG. 8. Treatment with Cas9 mRNA led to 58.2% editing of the Ttr gene. Animals treated with BC22n mRNA in the absence of UGI mRNA in trans displayed 29.26% C-to-T conversions and 28% indel, while those treated with BC22n and UGI mRNA showed a higher C-to-T editing purity (56.67% C-to-T conversions and only 5.6% indel).

Lowering the lipid dose from 0.3 to 0.1 mg/kg led to lower editing levels 31.39% C-to-T conversions and 3.67% indel).

TABLE 16
Ttr editing levels in mice treated with different LNP
combinations

| | | | mRNA(s) | | | | |
|---|---|---|---|---|---|---|---|
| | | | | Cas9, | | BC22n, | BC22n, |
| | | | TSS | UGI | BC22n | UGI | UGI |
| | | | Dose (editor + sgRNA) | | | | |
| | | | | 0.3 | 0.3 | 0.3 | 0.1 |
| | | | 0 | mg/kg | mg/kg | mg/kg | mg/kg |
| % Ttr | C-to- | Average | 0.2% | 0.1% | 29.3% | 56.7% | 31.4% |
| Editing | T | SD | 0.0% | 0.1% | 5.7% | 2.9% | 4.9% |
| | C-to- | Average | 0.5% | 0.3% | 8.1% | 1.8% | 1.7% |
| | A/G | SD | 0.1% | 0.1% | 1.3% | 0.2% | 0.1% |
| | Indel | Average | 0.2% | 58.2% | 28.0% | 5.7% | 3.7% |
| | | SD | 0.0% | 4.0% | 5.0% | 0.5% | 0.1% |
| | | SD | 14.1% | 6.7% | 9.6% | 3.5% | 9.0% |

(SD = standard deviation).

### 5.2 Whole Transcriptome Sequencing

Liver punches were mixed with 800 μL of TRIzol reagent (Thermo Fisher Scientific, Cat No. 15596026) in Lysing Matrix D tubes (MPBio, Cat. No. 116913100) which contain ceramic beads. Tissue was homogenized in a bead beater for 45 seconds and transferred to ice. Lysing Matrix D tubes were spun down at maximum speed for 5 min at 4C and ˜600 μL of TRIzol (without tissue debris) was mixed with an equal volume of absolute ethanol. The mixture was loaded in the Directzol RNA miniprep column (Zymo Research, Cat No. R2051) and RNA was extracted following the manufacturer's protocol. Purified RNA samples were quantified in a NanoDrop™ 8000 spectrophotometer (Thermo Fisher Scientific) and diluted to 41.67 ng/μL using nuclease-free water. From each experimental group, 2 samples were randomly chosen for further transcriptomic analysis. 500 ng (12 μL) of purified total RNA were depleted of ribosomal RNA (rRNA) components using the NEBNext® rRNA Depletion Kit (New England Biolabs, Cat. No. E6350L) according to the manufacturer's instructions. rRNA-depleted samples were converted into double-stranded DNA libraries using NEBNext® Ultra™ II Directional RNA Library Prep Kit for Illumina® (New England Biolabs, Cat No. E7765S) following the manufacturer's protocol. Amplified libraries were quantified in a Qubit 4 fluorometer and the average fragment size of each library was obtained by capillary electrophoresis. Libraries were pooled at an equimolar concentration of 4 nM and pair-end sequenced using a high-output 300-cycle kit (Illumina, Cat No. 20024908) in a NextSeq550 sequencing platform (Illumina).

Data Processing for Differential Gene Expression Analysis

Sequencing reads in FASTQ format were generated and demultiplexed using the bcl2fastq program (Illumina, v2.20). Reads were assigned to a sample if the Hamming distance (Hamming, R. W. Bell Syst. Tech. J. 29, 147-160) between each index read and the sample indexes was less than or equal to one. The sequencing quality was examined with FastQC program (v0.11.9) (Andrews S. Babraham Inst.). Ribosomal RNA reads were identified by aligning all reads to human rRNA sequences (GenBank U13369.1) with Bowtie2 (v2.3.5.1) (Langmead, B. and Salzberg, S. L. Nat. Methods 9, 357-359). Transcriptome quantification was performed using Salmon (v0.14.1) (Patro R., et al. Nat. Methods 14, 417-419) with non-ribosomal RNA reads. Differential gene expression analysis was carried out using DESeq2 (v1.26.0) (Love, M. I., et al. Genome Biol. 15, 550) on the outputs of Salmon. Genes or transcripts with Benjamini-Hochberg adjusted p-values less than 0.05 were determined to be differentially expressed.

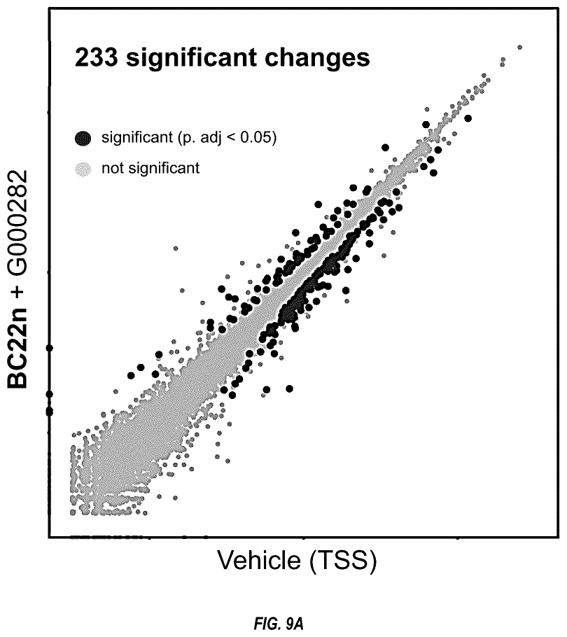

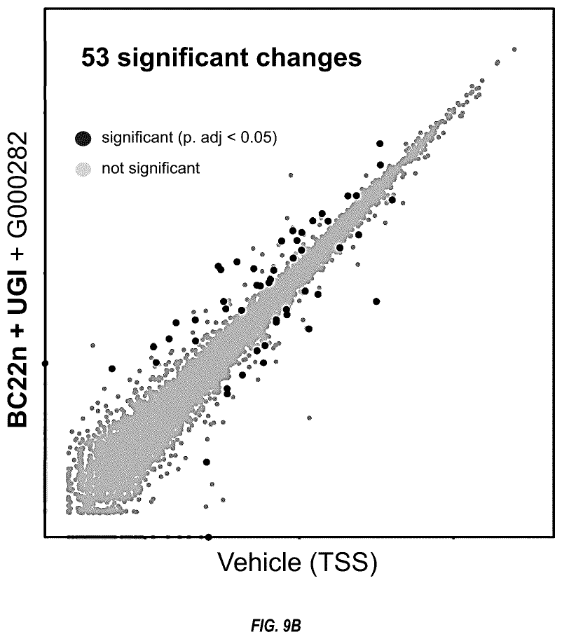

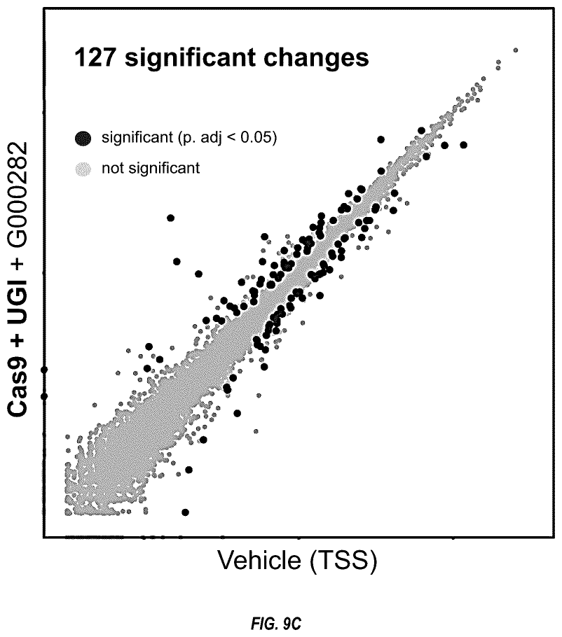

RNA-Seq analysis revealed that treatment with BC22n mRNA and UGI mRNA in trans led to only 53 differentially expressed genes, compared to 223 events in those animals treated with BC22n alone (i.e. no UGI mRNA) and 127 events in animals treated with both Cas9 and UGI mRNA in trans (FIGS. 9A-9C).

### Example 6—Editing in T Cells with UGI in Trans

6.1 Editing in T Cells

T cells were edited at the CIITA locus with UGI in trans and either BC22n or Cas9 to assess the impact on editing type on MHC II antigens.

Healthy human donor apheresis was obtained commercially (Hemacare), and cells were washed and re-suspended in CliniMACS® PBS/EDTA buffer (Miltenyi Biotec Cat. No. 130-070-525) on the LOVO device. T cells were isolated via positive selection using CD4 and CD8 magnetic beads (Miltenyi Biotec Cat. No. 130-030-401/130-030-801) using the CliniMACS® Plus and CliniMACS® LS disposable kit. T cells were aliquoted into vials and cryopreserved in a 1:1 formulation of Cryostor® CS10 (StemCell Technologies Cat. No. 07930) and Plasmalyte A (Baxter Cat. No. 2B2522X) for future use. Upon thaw, T cells were plated at a density of 1.0×10\hat{ }6 cells/mL in T cell basal media composed of X-VIVO 15™ serum-free hematopoietic cell medium (Lonza Bioscience) containing 5% (v/v) of fetal bovine serum, 50 μM of 2-Mercaptoethanol, 10 mM of N-Acetyl-L-(+)-cysteine, 10 U/mL of Penicillin-Streptomycin, in addition to 1× cytokines (200 U/mL of recombinant human interleukin-2, 5 μg/mL of recombinant human interleukin-7 and 5 μg/mL of recombinant human interleukin-15). T-cells were activated with TransAct™ (1:100 dilution, Miltenyi Biotec). Cells were expanded in T cell basal media containing TransAct™ for 72 hours prior to electroporation.

### Electroporation of T Cells

A solution containing mRNAs encoding Cas9 (SEQ ID NO. 11), BC22n (SEQ ID NO: 2) or UGI (SEQ ID NO: 26) was prepared in sterile water. 50 μM CIITA sgRNAs were removed from their storage plates and denatured for 2 minutes at 95° C. before cooling on ice. Seventy-two hours post activation, T cells were harvested, centrifuged, and resuspended at a concentration of 12.5×10\hat{ }6 T cells/mL in P3 electroporation buffer (Lonza). For each well to be electroporated, 1×10\hat{ }5 T cells were mixed with 200 ng of editor mRNA, 200 ng of UGI mRNA and 20 pmols of sgRNA as described in Table 17 in a final volume of 20 uL of P3 electroporation buffer. This mix was transferred in triplicate to a 96-well Nucleofector™ plate and electroporated using the manufacturer's pulse code. Electroporated T cells were immediately rested in 80 μL of T cell basal media without cytokines for 10 minutes before being transferred to a new flat-bottom 96-well plate containing an additional 100 μL of T cell basal media supplemented with 2× cytokines. The resulting plate was incubated at 37° C. for 4 days. After 96 hours, T cells were diluted 1:3 into fresh T cell basal media with 1× cytokines. Electroporated T cells were subsequently cultured for 3 additional days and were collected for flow cytometry analysis, NGS sequencing, and transcriptomics as described in Example 1.

### Flow Cytometry and NGS Sequencing

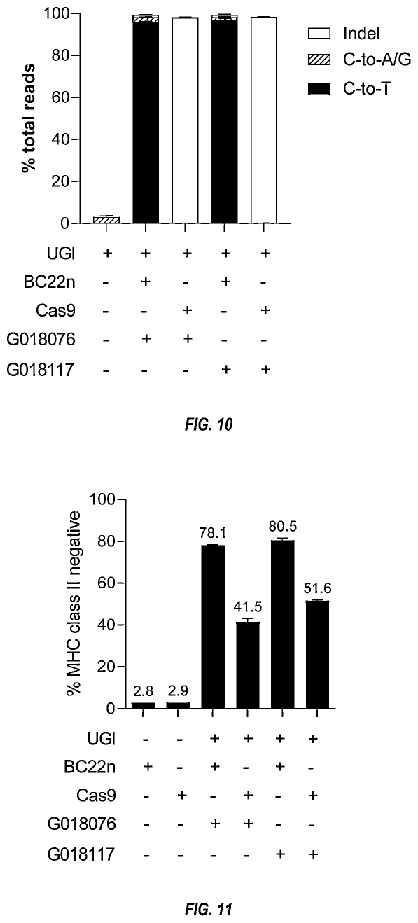

On day 7 post-editing, T cells were phenotyped by flow cytometry to determine MHC class II protein expression. Briefly, T cells were incubated in a cocktail of antibodies targeting HLA-DR, DQ, DP-PE (BioLegend® Cat. No. 361704) and Isotype Control-PE (BioLegend® Cat. No. 400234). Cells were subsequently washed, processed on a Cytoflex flow cytometer (Beckman Coulter) and analyzed using the FlowJo software package. T cells were gated based on size, shape, viability, and MHC II expression. DNA samples were subjected to PCR and subsequent NGS analysis, as described in Example 1. Table 17 and FIG. 10 show CIITA gene editing. For both Cas9 and BC22n conditions, total editing went to near completion, above 95%. Table 17 and FIG. 11 show MHC class II protein expression following electroporation with UGI mRNA combined with Cas9 or BC22n mRNA. For G018117, editing with BC22n lead to 80.50% MHC II negative cells, P while editing with Cas9 lead to 51.63% MHC II antigen negative cells.

TABLE 17
CIITA editing in T cells presented at a percentage of total
NGS reads; Flow cytometry assessment of the percentage
of cells lacking surface markers HLA DQ/DP/DR

| | | % C-to-T | | % C-to-A/G | | % Indel | | % MHC II neg | | |
|---|---|---|---|---|---|---|---|---|---|---|
| Editor | Guide | Mean | SD | Mean | SD | Mean | SD | Mean | SD | N |
| Cas9 | G018117 | 0.0 | 0.0 | 0.1 | 0.0 | 98.0 | 0.2 | 70.3 | 1.5 | 3 |
| | G018118 | 0.0 | 0.0 | 0.1 | 0.1 | 97.5 | 0.5 | 73.7 | 0.5 | 3 |
| | G018120 | 0.1 | 0.1 | 0.2 | 0.0 | 92.3 | 0.3 | 83.1 | 1.1 | 3 |
| | G018076 | 0.0 | 0.0 | 0.1 | 0.0 | 98.3 | 0.1 | 62.8 | 1.1 | 3 |
| | G018100 | 0.0 | 0.0 | 0.1 | 0.0 | 98.9 | 0.2 | 84.1 | 1.3 | 3 |
| | G018091 | 0.0 | 0.0 | 0.0 | 0.0 | 99.4 | 0.2 | 96.2 | 0.4 | 3 |
| | No guide | not reported | | | | | | 10.5 | 0.0 | 1 |
| BC22n | G018117 | 95.7 | 0.2 | 2.3 | 0.1 | 1.1 | 0.1 | 99.2 | 0.1 | 3 |
| | G018118 | 95.8 | 0.3 | 2.3 | 0.1 | 1.2 | 0.3 | 99.0 | 0.1 | 3 |
| | G018120 | 95.7 | 0.5 | 2.6 | 0.4 | 0.7 | 0.1 | 99.0 | 0.1 | 3 |
| | G018076 | 96.5 | 0.4 | 1.2 | 0.2 | 1.1 | 0.3 | 98.5 | 0.2 | 3 |
| | G018100 | 90.7 | 0.3 | 2.8 | 0.3 | 5.1 | 0.7 | 95.7 | 0.6 | 3 |
| | G018091 | 95.4 | 0.6 | 2.8 | 0.1 | 1.1 | 0.3 | 98.7 | 0.2 | 3 |

| | | | | |
|---|---|---|---|---|
| No guide | not reported | 10.8 | 0.0 | 1 |

### 6.2—Gene Expression Analysis in T Cells

### Whole Transcriptome Sequencing

On day 7 post-editing, T cells treated with G018117 and G18078 were harvested and preserved at −80 C for future processing. Total RNA was extracted from samples in TRIzol™ reagent using the Direct-zol RNA microprep kit (Zymo Research, Cat No. R2062) following the manufacturer's protocol. Purified RNA samples were quantified in a NanoDrop™ 8000 spectrophotometer (Thermo Fisher Scientific) and diluted to 41.67 ng/μL using nuclease-free water. From each experimental triplicate shown in FIGS. 13A-15B, samples per group were randomly chosen for transcriptomic analysis. 500 ng (12 μL) of purified total RNA were depleted of ribosomal RNA (rRNA) components using the NEBNext® rRNA Depletion Kit (New England Biolabs, Cat. No. E6350L) according to the manufacturer's instructions. rRNA-depleted samples were converted into double-stranded DNA libraries using NEBNext® Ultra™ II Directional RNA Library Prep Kit for Illumina® (New England Biolabs, Cat No. E7765S) following the manufacturer's protocol. Amplified libraries were quantified in a Qubit 4 fluorometer and the average fragment size of each library was obtained by capillary electrophoresis. Libraries were pooled at an equimolar concentration of 4 nM and pair-end sequenced using a high-output 300-cycle kit (Illumina, Cat No. 20024908) in a NextSeq550 sequencing platform (Illumina).

### Data Processing for Differential Gene Expression Analysis

Sequencing reads in FASTQ format were generated and demultiplexed using the bcl2fastq program (Illumina, v2.20). Reads were assigned to a sample if the Hamming distance (Hamming, R. W. Bell Syst. Tech. J. 29, 147-160) between each index read and the sample indexes was less than or equal to one. The sequencing quality was examined with FastQC program (v0.11.9) (Andrews S. Babraham Inst.). Ribosomal RNA reads were identified by aligning all reads to human rRNA sequences (GenBank U13369.1) with Bowtie2 (v2.3.5.1) (Langmead, B. and Salzberg, S. L. Nat. Methods 9, 357-359). Transcriptome quantification was performed using Salmon (v0.14.1) (Patro R., et al. Nat. Methods 14, 417-419) with non-ribosomal RNA reads. Differential gene expression analysis was carried out using DESeq2 (v1.26.0) (Love, M. I., et al. Genome Biol. 15, 550) on the outputs of Salmon. Genes or transcripts with Benjamini-Hochberg adjusted p-values less than 0.05 were determined to be differentially expressed. Lists of differentially expressed genes were analyzed in terms of gene ontology using Metascape (Zhou, Y., et al. Nat. Comm. 10, 1523). Protein-protein interactions were determined using the BioGrid, InWeb_IM and OmniPath8 databases (Li, T., et al. Nat. Methods 14, 61-64; Stark, C., et al. Nucleic Acids Res. 34, 535-539; Tilrei, D., et al. Nat. Methods 13, 966-967). Densely connected networks were identified using the molecular complex detection (MCODE) algorithm (Bader, G. D., et al. BMC Bioinformatics 4, 1-27) and the three best-scoring terms by p-value were retained as the functional description of the corresponding network components.

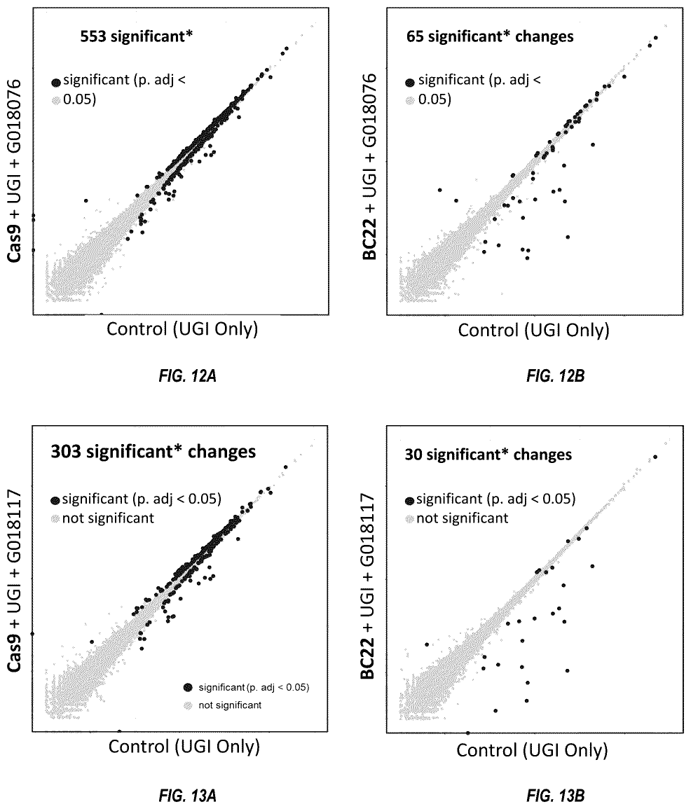

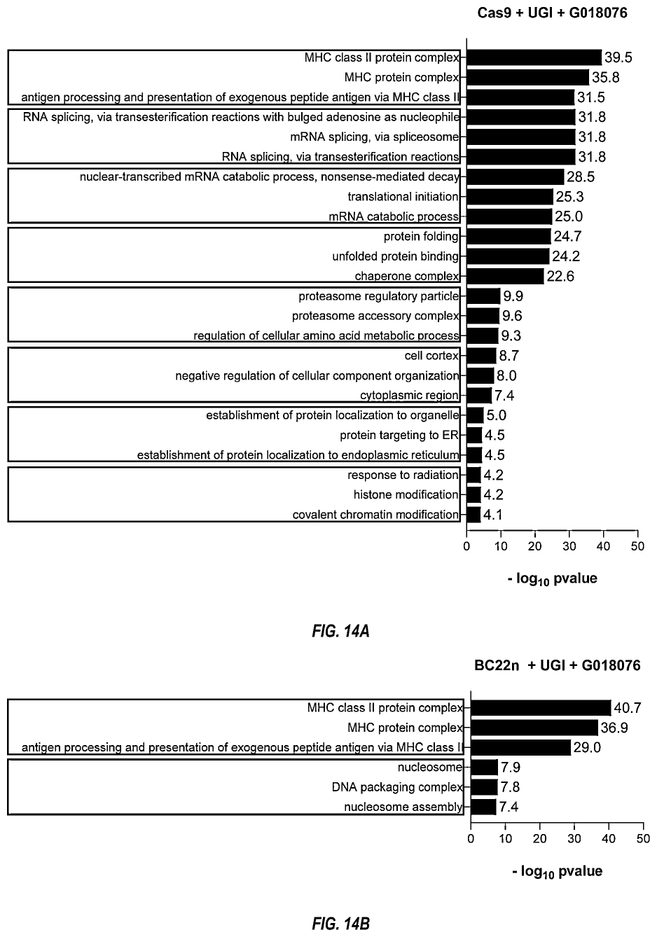

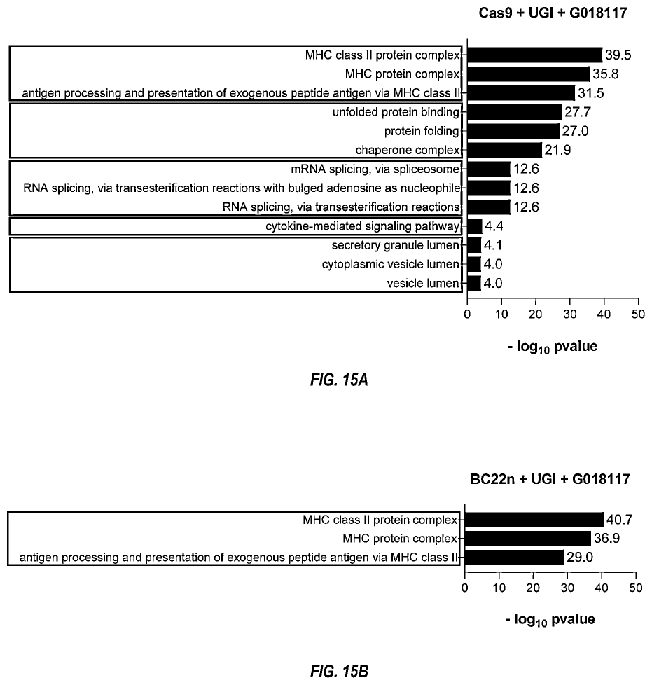

Compared to samples treated with Cas9 mRNA, T cells electroporated with BC22n mRNA displayed a significantly stronger downregulation of MHC class II genes and the HLA-associated CD74 gene (Table 18 and 19). Minimal effects on class I MHC genes were observed (Table 20 and Table 21). In terms of transcriptome-wide differential gene expression events, treatment with BC22n mRNA led to fewer differentially expressed genes (p. adjusted<0.05) when compared to Cas9 mRNA. In T cells electroporated with sgRNA G018076, a total of 553 and 65 differential gene expression events were observed for Cas9 and BC22n mRNA treatments, respectively (FIGS. 12A-12B). A similar trend was observed in T cells electroporated with sgRNA G018117, which displayed 303 and 30 differential gene expression events when treated with Cas9 and BC22n mRNA, respectively (FIGS. 13A-13B). Fewer protein-protein interaction networks were identified among the list of differentially expressed genes in T cells treated with BC22n mRNA when compared to those treated with Cas9 mRNA (FIGS. 14A-14B and 15A-15B).

TABLE 18
Differential gene expression of class II HLA genes in T cells

| | G018076 sgRNA | | | | G018117 sgRNA | | | |
|---|---|---|---|---|---|---|---|---|
| | | | BC22n | | Cas9 | | | |
| | Cas9 vs control | | vs control | | vs control | | BC22n vs control | |
| | Fold | | Fold | | Fold | | Fold | |
| Gene | change | p. adj. | change | p. adj. | change | p. adj. | change | p. adj. |
| CD74 | 0.446 | *** | 0.116 | *** | 0.360 | *** | 0.100 | *** |
| HLA-DMA | 0.457 | *** | 0.150 | *** | 0.356 | *** | 0.146 | *** |
| HLA-DMB | 0.363 | *** | 0.113 | *** | 0.267 | *** | 0.091 | *** |
| HLA-DOA | 0.450 | *** | 0.299 | *** | 0.420 | *** | 0.280 | *** |
| HLA-DPA1 | 0.474 | *** | 0.181 | *** | 0.385 | *** | 0.170 | *** |
| HLA-DPB1 | 0.381 | *** | 0.087 | *** | 0.300 | *** | 0.073 | *** |
| HLA-DQA1 | 0.316 | *** | 0.017 | *** | 0.214 | *** | 0.009 | *** |
| HLA-DQA2 | 0.215 | *** | 0.007 | *** | 0.221 | *** | 0.011 | *** |
| HLA-DQB1 | 0.383 | *** | 0.069 | *** | 0.290 | **** | 0.069 | *** |
| HLA-DQB1- | 0.323 | *** | 0.102 | *** | 0.311 | *** | 0.097 | *** |
| AS1 | | | | | | | | |
| HLA-DRA | 0.288 | *** | 0.004 | *** | 0.205 | *** | 0.002 | *** |
| HLA-DRB1 | 0.287 | *** | 0.029 | *** | 0.207 | *** | 0.027 | *** |
| HLA-DRB3 | 0.268 | *** | 0.012 | *** | 0.219 | *** | 0.004 | *** |
| HLA-DRB4 | 0.282 | *** | 0.024 | *** | 0.224 | *** | 0.026 | *** |

(ns = not significant,
* = p. adj. < 0.05,
** = p. adj. < 0.01,
*** = p. adj. < 0.001).
For transcript quantification data, refer to Table 19.

TABLE 19
Transcript quantification of the expression of class II
HLA genes in T cells. Each square contains the
average number of transcripts from a given
gene per one million of mRNA molecules. For statistical
significance, please refer to Table 19.

| | | G0188117 | | G018076 | |
|---|---|---|---|---|---|
| Guide | none | Cas9 | BC22n | Cas9 | BC22n |
| mRNAs | UGI | UGI | UGI | UGI | UGI |
| CD74 | 385 | 154 | 38 | 174 | 45 |
| HLA-DMA | 27 | 11 | 4 | 12 | 4 |
| HLA-DMB | 11 | 3 | 1 | 4 | 1 |
| HLA-DOA | 3 | 2 | 1 | 2 | 1 |
| HLA-DPA1 | 70 | 30 | 12 | 34 | 13 |
| HLA-DPB1 | 16 | 5 | 1 | 6 | 1 |
| HLA-DQA1 | 12 | 3 | 0 | 4 | 0 |
| HLA-DQA2 | 3 | 1 | 0 | 1 | 0 |
| HLA-DQB1 | 60 | 19 | 4 | 23 | 4 |
| HLA-DQB1-AS1 | 9 | 3 | 1 | 3 | 1 |
| HLA-DRA | 120 | 27 | 0 | 35 | 1 |
| HLA-DRB1 | 117 | 27 | 3 | 34 | 3 |
| HLA-DRB3 | 17 | 4 | 0 | 5 | 0 |
| HLA-DRB4 | 13 | 3 | 0 | 4 | 0 |

TABLE 20
Differential gene expression of class I HLA genes in T cells
harvested 7 days post-treatment with different mRNA
combinations and CIITA-targeting sgRNAs

| | G018076 sgRNA | | | | G018117 sgRNA | | | |
|---|---|---|---|---|---|---|---|---|
| | Cas9 | | BC22n | | Cas9 | | BC22n | |
| | vs control | | vs control | | vs control | | vs control | |
| | Fold | p. | Fold | p. | Fold | p. | Fold | p. |
| Gene | change | adj. | change | adj. | change | adj. | change | adj. |
| HLA-A | 0.995 | ns | 0.910 | * | 0.969 | ns | 0.926 | ns |
| HLA-B | 1.001 | ns | 0.881 | ** | 1.043 | ns | 0.913 | ns |
| HLA-C | 1.013 | ns | 0.917 | ns | 0.995 | ns | 0.922 | ns |
| HLA-E | 0.925 | ns | 0.919 | ns | 0.870 | *** | 0.949 | ns |
| HLA-F | 0.897 | ns | 0.812 | ** | 0.930 | ns | 0.891 | ns |

(ns = not significant,
* = p. adj. < 0.05,
** = p. adj. < 0.01,
*** = p. adj. < 0.001).
For transcript quantification data, refer to Table 21.

TABLE 21
Transcript quantification of the expression of class I HLA
genes in T cells. Each square contains the average
number of transcripts from a given gene
per one million of mRNA molecules. For statistical
significance, please refer to Table 20.

| | | G0188117 | | G018076 | |
|---|---|---|---|---|---|
| Guide | none | Cas9 | BC22n | Cas9 | BC22n |
| mRNAs | UGI | UGI | UGI | UGI | UGI |
| HLA-A | 880 | 947 | 797 | 887 | 814 |
| HLA-B | 457 | 528 | 407 | 463 | 409 |
| HLA-C | 479 | 528 | 430 | 490 | 445 |
| HLA-E | 166 | 161 | 154 | 156 | 155 |
| HLA-F | 68 | 70 | 59 | 61 | 56 |

### Example 7—Editing Assessment at APOBEC3A Hotspots

T Cell Preparation

Healthy human donor apheresis was obtained commercially (Hemacare), and cells were washed and re-suspended in CliniMACS® PBS/EDTA buffer (Miltenyi Biotec Cat. No. 130-070-525) on the LOVO device. T cells were isolated via positive selection using CD4 and CD8 magnetic beads (Miltenyi Biotec Cat. No. 130-030-401/130-030-801) using the CliniMACS® Plus and CliniMACS® LS disposable kit. T cells were aliquoted into vials and cryopreserved in a 1:1 formulation of Cryostor® CS10 (StemCell Technologies Cat. No. 07930) and Plasmalyte A (Baxter Cat. No. 2B2522X) for future use. Upon thaw, T cells were plated at a density of 1.0×10\hat{ }6 cells/mL in T cell basal media composed of X-VIVO 15™ serum-free hematopoietic cell medium (Lonza Bioscience) containing 5% (v/v) of fetal bovine serum, 50 μM of 2-Mercaptoethanol, 10 mM of N-Acetyl-L-(+)-cysteine, 10 U/mL of Penicillin-Streptomycin, in addition to 1× cytokines (200 U/mL of recombinant human interleukin-2, 5 μg/mL of recombinant human interleukin-7 and 5 μg/mL of recombinant human interleukin-15). T-cells were activated with TransAct™ (1:100 dilution, Miltenyi Biotec). Cells were expanded in T cell basal media containing TransAct™ for 72 hours prior to electroporation.

mRNA and sgRNA Electroporation of T Cells

A solution containing mRNA encoding Cas9 (SEQ ID NO: 11), BC22n (SEQ ID NO: 2) or UGI (SEQ ID NO: 26) was prepared in sterile water. A 50 μM TRAC targeting sgRNA (G016017) was removed from its storage plate and denatured for 2 minutes at 95° C. before cooling on ice. Seventy-two hours post activation, T cells were harvested, centrifuged, and resuspended at a concentration of 12.5×10\hat{ }6 T cells/mL in P3 electroporation buffer (Lonza). For each well to be electroporated, 1×10\hat{ }5 T cells were mixed with 200 ng of each mRNA and 20 pmols of sgRNA in a final volume of 20 μL of P3 electroporation buffer. The T cell mix was transferred in triplicate to a 96-well Nucleofector™ plate and electroporated using the manufacturer's pulse code. Electroporated T cells were immediately rested in 80 μL of T cell basal media without cytokines for 10 minutes before being transferred to a new flat-bottom 96-well plate containing an additional 100 μL of T cell basal media supplemented with 2× cytokines. The resulting plate was incubated at 37° C. for 4 days. After 96 hours, T cells were diluted 1:3 into fresh T cell basal media with 1× cytokines. Electroporated T cells were subsequently cultured for 3 additional days and were collected for NGS sequencing.

NGS Sequencing

On day 7 post-editing, DNA samples were subjected to PCR and subsequent NGS analysis, as described in Example 1. This study characterized the on-target TRAC locus in addition to 10 genomic loci previously described as mutational hotspots in tumor samples positive for APOBEC enzymes (Buisson et al., 2019). The chromosomal location of these sites is listed on Table 22.

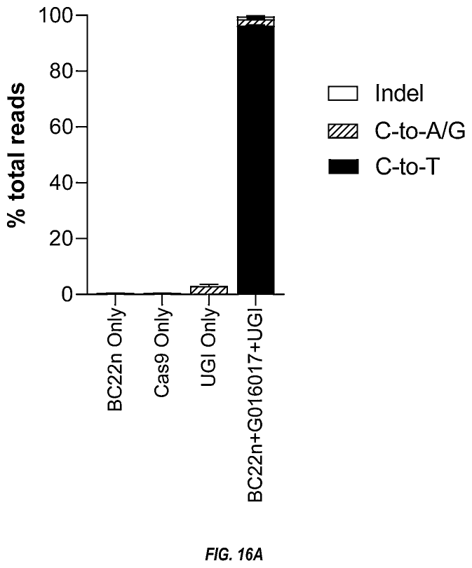

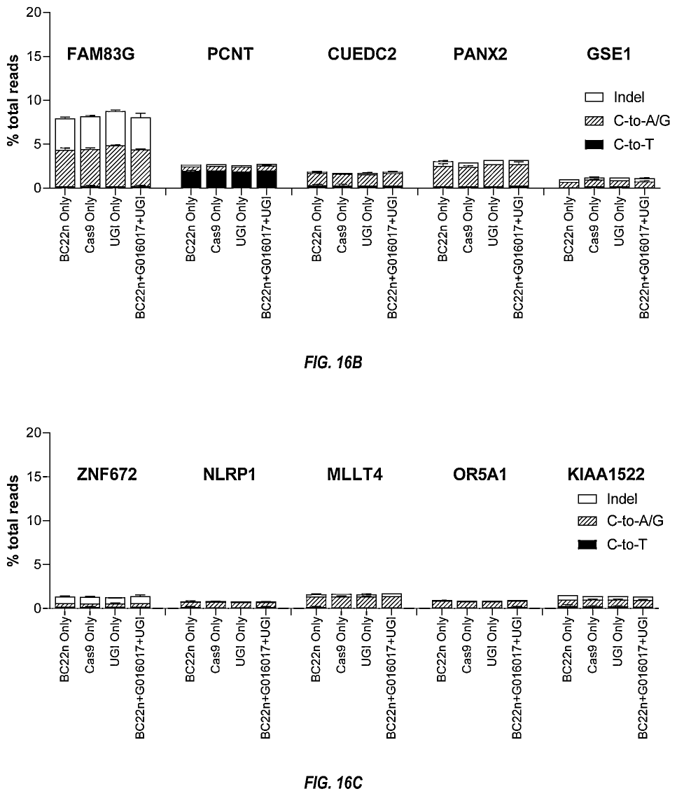

Tables 23, 24, and 25 show C-to-T, C-to-A/G and indel editing levels in the on-target locus and predicted APOBEC hotspot sites for all sample groups. A graphical representation of these results in shown in FIGS. 16A-16C. Compared to control samples treated with Cas9 mRNA, T cells electroporated with BC22n mRNA, UGI mRNA or BC22n mRNA in addition to both UGI mRNA and the sgRNA G016017 did not display any significant changes in the editing profile of 10 genomic loci previously reported as mutational hotspots in tumor samples positive for APOBEC enzymes (Buisson R., et al. (2019). Science 364, 1-8). High levels of deamination at the on-target TRAC locus were observed in samples treated with BC22n mRNA, UGI mRNA and sgRNA G016017, but absent in those treated with BC22n mRNA alone, Cas9 mRNA alone or UGI mRNA alone.

TABLE 22
List of mutational hotspots evaluated in this study, their
Entrez gene IDs and genomic locations.

| | Entrez | | Genomic | |
|---|---|---|---|---|
| Gene symbol | gene ID | Chromosome | location | Strand |
| FAM83G | 644815 | 17 | 18,907,093 | − |
| PCNT | 5116 | 21 | 47,783,764 | − |
| CUEDC2 | 79004 | 10 | 104,184,490 | − |
| PANX2 | 56666 | 22 | 50,615,649 | − |
| GSE1 | 23199 | 16 | 85,691,140 | − |
| ZNF672 | 79894 | 1 | 249,142,293 | + |
| NLRP1 | 22861 | 17 | 5,461,801 | + |
| MLLT4/AFDN | 4301 | 6 | 168,352,586 | + |
| OR5A1 | 219982 | 11 | 59,211,279 | + |
| KIAA1522 | 57648 | 1 | 33,237,497 | + |

TABLE 23
Levels of C-to-T base conversion in the on-target TRAC locus in
addition to 10 previously described APOBEC hotspots in the human genome.

| | C-to-T Editing | | | | | | | |
|---|---|---|---|---|---|---|---|---|
| | | | | | | | BC22n mRNA + | |
| | | | | | | | UGI mRNA + | |
| | Cas9 mRNA | | BC22n mRNA | | UGI mRNA | | G016017 | |
| Gene | Mean | SD | Mean | SD | Mean | SD | Mean | SD |
| TRAC (on- | 0.14% | 0.02% | 0.13% | 0.01% | 0.19% | 0.02% | 96.26% | 0.25% |
| target) | | | | | | | | |
| FAM83G | 0.23% | 0.05% | 0.22% | 0.03% | 0.22% | 0.04% | 0.28% | 0.05% |
| PCNT | 2.02% | ND | 1.97% | 0.09% | 1.92% | 0.04% | 1.99% | 0.00% |
| CUEDC2 | 0.32% | 0.12% | 0.36% | 0.04% | 0.27% | 0.01% | 0.28% | 0.00% |
| PANX2 | 0.16% | 0.01% | 0.19% | 0.00% | 0.21% | ND | 0.26% | 0.01% |
| GSE1 | 0.20% | 0.03% | 0.11% | ND | 0.15% | ND | 0.12% | 0.02% |
| ZNF672 | 0.15% | 0.02% | 0.19% | 0.03% | 0.20% | 0.01% | 0.18% | 0.02% |
| NLRP1 | 0.14% | 0.00% | 0.15% | 0.01% | 0.15% | 0.00% | 0.16% | 0.02% |
| MLLT4 | 0.12% | 0.01% | 0.13% | 0.03% | 0.13% | 0.01% | 0.14% | 0.00% |
| OR5A1 | 0.12% | 0.03% | 0.13% | 0.01% | 0.13% | 0.02% | 0.14% | 0.04% |
| KIAA1522 | 0.24% | 0.03% | 0.29% | 0.08% | 0.25% | 0.02% | 0.24% | 0.01% |

SD = standard deviation;
ND = not determined.

TABLE 24
Levels of C-to-A/G transversions in the on-target TRAC locus in
addition to 10 previously described APOBEC hotspots in the human genome.

| | C-to-A/G Editing | | | | | | | |
|---|---|---|---|---|---|---|---|---|
| | | | | | | | BC22n mRNA + | |
| | | | | | | | UGI mRNA + | |
| | Cas9 mRNA | | BC22n mRNA | | UGI mRNA | | G016017 | |
| Gene | Mean | SD | Mean | SD | Mean | SD | Mean | SD |
| TRAC (on- | 0.29% | 0.03% | 0.30% | 0.03% | 2.73% | 0.69% | 2.38% | 0.20% |
| target) | | | | | | | | |
| FAM83G | 4.22% | 0.11% | 4.11% | 0.22% | 4.66% | 0.03% | 4.13% | 0.06% |
| PCNT | 0.45% | ND | 0.53% | 0.01% | 0.52% | 0.13% | 0.53% | 0.04% |
| CUEDC2 | 1.28% | 0.01% | 1.34% | 0.11% | 1.27% | 0.20% | 1.36% | 0.01% |
| PANX2 | 2.18% | 0.15% | 2.32% | 0.24% | 2.48% | ND | 2.37% | 0.27% |
| GSE1 | 0.72% | 0.06% | 0.58% | ND | 0.70% | ND | 0.66% | 0.03% |
| ZNF672 | 0.39% | 0.03% | 0.42% | 0.02% | 0.36% | 0.04% | 0.39% | 0.02% |
| NLRP1 | 0.62% | 0.06% | 0.62% | 0.01% | 0.60% | 0.02% | 0.57% | 0.08% |
| MLLT4 | 1.27% | 0.09% | 1.21% | 0.01% | 1.26% | 0.03% | 1.32% | 0.03% |
| OR5A1 | 0.72% | 0.02% | 0.75% | 0.06% | 0.70% | 0.04% | 0.73% | 0.03% |
| KIAA1522 | 0.72% | 0.08% | 0.70% | 0.07% | 0.76% | 0.04% | 0.74% | 0.04% |

SD = standard deviation;
ND = not determined.

TABLE 25
Levels of insertions and deletions (indel) in the on-target TRAC locus
in addition to 10 previously described APOBEC hotspots in the human genome.

| | Indel | | | | | | | |
|---|---|---|---|---|---|---|---|---|
| | | | | | | | BC22n mRNA + | |
| | | | | | | | UGI mRNA + | |
| | Cas9 mRNA | | BC22n mRNA | | UGI mRNA | | G016017 | |
| Gene | Mean | SD | Mean | SD | Mean | SD | Mean | SD |
| TRAC (on- | 0.08% | 0.00% | 0.11% | 0.01% | 0.11% | 0.02% | 0.95% | 0.26% |
| target) | | | | | | | | |
| FAM83G | 3.78% | 0.10% | 3.63% | 0.11% | 3.90% | 0.13% | 3.67% | 0.50% |
| PCNT | 0.11% | ND | 0.23% | 0.00% | 0.18% | 0.02% | 0.21% | 0.02% |
| CUEDC2 | 0.09% | 0.05% | 0.14% | 0.03% | 0.13% | 0.03% | 0.14% | 0.02% |
| PANX2 | 0.47% | 0.01% | 0.54% | 0.10% | 0.26% | ND | 0.52% | 0.02% |
| GSE1 | 0.23% | 0.06% | 0.13% | ND | 0.15% | ND | 0.36% | 0.05% |
| ZNF672 | 0.71% | 0.06% | 0.80% | 0.08% | 0.72% | 0.01% | 0.78% | 0.15% |
| NLRP1 | 0.07% | 0.03% | 0.07% | 0.03% | 0.08% | 0.01% | 0.07% | 0.01% |
| MLLT4 | 0.26% | 0.00% | 0.25% | 0.01% | 0.25% | 0.02% | 0.27% | 0.01% |
| OR5A1 | 0.07% | 0.02% | 0.08% | 0.01% | 0.08% | 0.01% | 0.07% | 0.01% |
| KIAA1522 | 0.38% | 0.01% | 0.46% | 0.02% | 0.42% | 0.02% | 0.41% | 0.00% |

SD = standard deviation;
ND = not determined.

### Example 8—LNP Titration in T Cells with Fixed Ratio of BC22n:UGI

Using LNP delivery to activated human T cells, the potency of single-target and multi-target editing was assessed with either Cas9 or BC22n.

### 8.1 T Cell Preparation

Healthy human donor apheresis was obtained commercially (Hemacare), and cells were washed and re-suspended in CliniMACS® PBS/EDTA buffer (Miltenyi Biotec Cat. No. 130-070-525) on the LOVO device. T cells were isolated via positive selection using CD4 and CD8 magnetic beads (Miltenyi Biotec Cat. No. 130-030-401/130-030-801) using the CliniMACS® Plus and CliniMACS® LS disposable kit. T cells were aliquoted into vials and cryopreserved in a 1:1 formulation of Cryostor® CS10 (StemCell Technologies Cat. No. 07930) and Plasmalyte A (Baxter Cat. No. 2B2522X) for future use. Upon thaw, T cells were plated at a density of 1.0×10\hat{ }6 cells/mL in T cell basal media composed of X-VIVO 15™ serum-free hematopoietic cell medium (Lonza Bioscience) containing 5% (v/v) of fetal bovine serum, 50 μM of 2-Mercaptoethanol, 10 mM of N-Acetyl-L-(+)-cysteine, 10 U/mL of Penicillin-Streptomycin, in addition to 1× cytokines (200 U/mL of recombinant human interleukin-2, 5 μg/mL of recombinant human interleukin-7 and 5 μg/mL of recombinant human interleukin-15). T cells were activated with TransAct™ (1:100 dilution, Miltenyi Biotec). Cells were expanded in T cell basal media for 72 hours prior to LNP transfection.

### 8.2 T Cell Editing

Each RNA species, i.e. UGI mRNA, sgRNA or editor mRNA, was formulated separately in an LNP as described in Example 1. Editor mRNAs encoded either BC22n (SEQ ID NO: 5) or Cas9 (SEQ ID NO: 23). Guides targeting B2M (G015995), TRAC (G016017), TRBC1/2 (G016206) and CIITA (G018117 and G016086) were used either singly or in combination. Messenger RNA encoding UGI (SEQ ID NO: 26) is delivered in both Cas9 and BC22n arms of the experiment to normalize lipid amounts. Previous experiments have established UGI mRNA does not impact total editing or editing profile when used with Cas9 mRNA. LNPs were mixed to fixed total RNA weight ratios of 6:3:2 for editor mRNA, guide RNA, and UGI mRNA respectively as described in Table 26. In the 4-guide experiment described in Table 27, doses of LNPs with individual guides are decreased 4-fold to maintain the overall 6:3 editor mRNA:guide weight ratio and to allow comparison to individual guide potency based on total lipid delivery. LNP mixtures were incubated for 5 minutes at 37° C. in T cell basal media substituting 6% cynomolgus monkey serum (Bioreclamation IVT, Cat. CYN220760) for fetal bovine serum.

Seventy-two hours post activation, T cells were washed and suspended in basal T cell media. Pre-incubated LNP mix was added to the each well with 1×10e5 T cells/well. T cells were incubated at 37° C. with 5% C02 for the duration of the experiment. T cell media was changed 6 days and 8 days after activation and on tenth day post activation, cells were harvested for analysis by NGS and flow cytometry. NGS analysis was performed as described in Example 1.

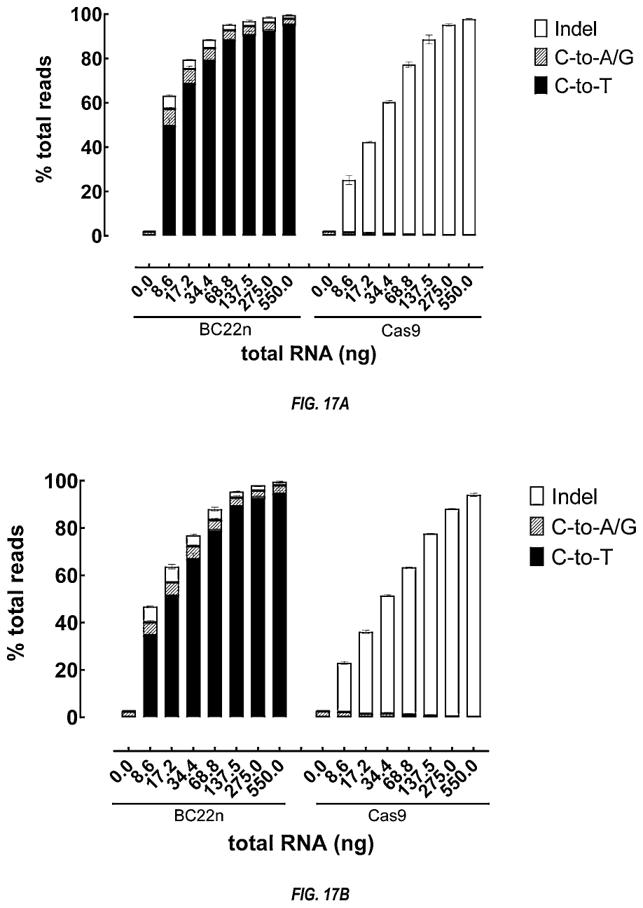

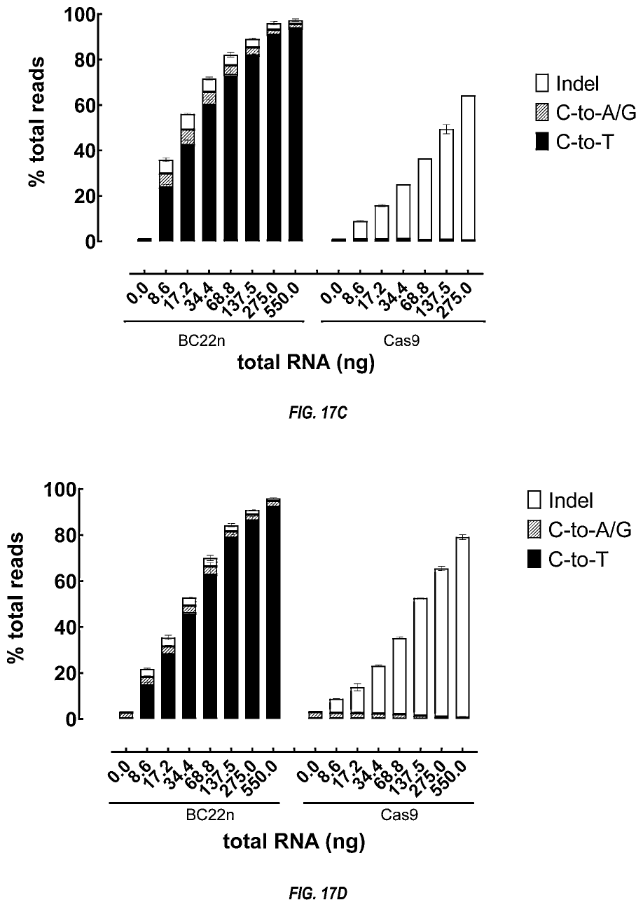

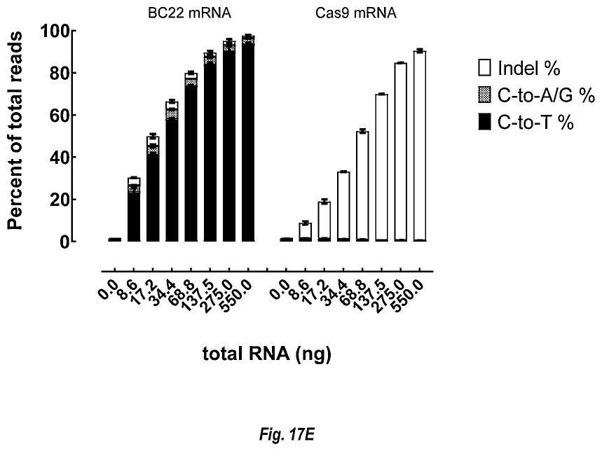

Table 26 and FIGS. 17A-E describe the editing profile of T cells when an individual guide was used for editing. Total editing and C to T editing showed direct, dose responsive relationships to increasing amounts of BC22n mRNA, UGI mRNA and guide across all guides tested. Indel and C conversions to A or G are in an inverse relationship with dose where lower doses resulted in a higher percentage of these mutations. In samples edited with Cas9, total editing and indel activity increase with the total RNA dose.

TABLE 26
Editing as a percent of total reads-single guide delivery

| | | Total | | | % C- | | | | |
|---|---|---|---|---|---|---|---|---|---|
| | | RNA | % C-to-T | | to-A/G | | % Indel | | |
| Guide | Editor | (ng) | mean | SD | mean | SD | mean | SD | N |
| G015995 | BC22n | 0.0 | 0.3 | 0.0 | 1.5 | 0.1 | 0.2 | 0.0 | 2 |
| B2M | | 8.6 | 49.5 | 3.5 | 7.7 | 0.6 | 6.0 | 0.4 | 2 |
| | | 17.2 | 68.5 | 1.7 | 6.7 | 1.3 | 4.3 | 0.1 | 2 |
| | | 34.4 | 79.0 | 0.9 | 5.7 | 0.3 | 3.8 | 0.0 | 2 |
| | | 68.8 | 88.2 | 0.8 | 4.6 | 0.0 | 2.5 | 0.2 | 2 |
| | | 137.5 | 90.6 | 1.8 | 4.1 | 0.4 | 2.2 | 0.5 | 2 |
| | | 275.0 | 92.6 | 0.8 | 3.7 | 0.3 | 2.2 | 0.3 | 2 |
| | | 550.0 | 95.2 | 0.4 | 2.8 | 0.0 | 1.6 | 0.2 | 2 |
| | Cas9 | 0.0 | 0.3 | 0.0 | 1.5 | 0.2 | 0.2 | 0.0 | 2 |
| | | 8.6 | 0.3 | 0.0 | 1.2 | 0.1 | 23.7 | 2.1 | 2 |
| | | 17.2 | 0.3 | 0.0 | 0.9 | 0.1 | 41.1 | 0.2 | 2 |
| | | 34.4 | 0.3 | 0.0 | 0.6 | 0.0 | 59.4 | 0.6 | 2 |
| | | 68.8 | 0.2 | 0.1 | 0.4 | 0.0 | 76.8 | 1.2 | 2 |
| | | 137.5 | 0.1 | 0.1 | 0.2 | 0.0 | 88.2 | 2.0 | 2 |
| | | 275.0 | 0.1 | 0.0 | 0.1 | 0.1 | 95.1 | 0.5 | 2 |
| | | 550.0 | 0.1 | 0.0 | 0.1 | 0.0 | 97.5 | 0.3 | 2 |
| G016017 | BC22n | 0.0 | 0.2 | 0.0 | 2.2 | 0.1 | 0.2 | 0.1 | 2 |
| TRAC | | 8.6 | 34.6 | 1.1 | 5.6 | 0.8 | 6.6 | 0.2 | 2 |
| | | 17.2 | 51.3 | 0.8 | 5.7 | 0.1 | 6.7 | 1.0 | 2 |
| | | 34.4 | 66.9 | 2.6 | 5.4 | 0.2 | 4.7 | 0.4 | 2 |
| | | 68.8 | 79.0 | 0.6 | 4.4 | 0.7 | 4.5 | 0.9 | 2 |
| | | 137.5 | 89.2 | 0.4 | 3.6 | 0.9 | 2.5 | 0.2 | 2 |
| | | 275.0 | 92.8 | 0.9 | 2.9 | 0.0 | 2.3 | 0.0 | 2 |
| | | 550.0 | 94.5 | 1.3 | 3.4 | 1.0 | 1.6 | 0.2 | 2 |
| | Cas9 | 0.0 | 0.2 | 0.0 | 2.3 | 0.1 | 0.1 | 0.0 | 2 |
| | | 8.6 | 0.2 | 0.0 | 2.1 | 0.2 | 20.7 | 0.5 | 2 |
| | | 17.2 | 0.1 | 0.0 | 1.4 | 0.0 | 34.6 | 0.7 | 2 |
| | | 34.4 | 0.1 | 0.0 | 1.5 | 0.4 | 49.8 | 0.4 | 2 |
| | | 68.8 | 0.1 | 0.0 | 1.0 | 0.0 | 62.3 | 0.1 | 2 |
| | | 137.5 | 0.1 | 0.0 | 0.6 | 0.1 | 77.0 | 0.1 | 2 |
| | | 275.0 | 0.0 | 0.0 | 0.3 | 0.0 | 87.8 | 0.2 | 2 |
| | | 550.0 | 0.0 | 0.0 | 0.2 | 0.0 | 93.8 | 0.6 | 2 |
| G016206 | BC22n | 0.0 | 0.4 | 0.1 | 0.6 | 0.1 | 0.1 | 0.1 | 2 |
| TRBC1/2 | | 8.6 | 23.7 | 1.3 | 6.1 | 0.0 | 6.1 | 0.8 | 2 |
| | | 17.2 | 42.4 | 2.2 | 6.8 | 0.1 | 6.8 | 0.3 | 2 |
| | | 34.4 | 60.1 | 2.2 | 5.7 | 0.3 | 5.9 | 0.7 | 2 |
| | | 68.8 | 73.2 | 4.2 | 4.3 | 0.1 | 4.7 | 1.1 | 2 |
| | | 137.5 | 81.7 | 0.8 | 3.6 | 0.2 | 3.7 | 0.4 | 2 |
| | | 275.0 | 91.0 | 1.7 | 2.3 | 0.1 | 2.8 | 0.8 | 2 |
| | | 550.0 | 93.6 | 1.9 | 2.0 | 0.2 | 1.7 | 0.6 | 2 |
| | Cas9 | 0.0 | 0.3 | 0.0 | 0.5 | 0.0 | 0.1 | 0.0 | 1 |
| | | 8.6 | 0.3 | 0.2 | 0.5 | 0.1 | 8.1 | 0.2 | 2 |
| | | 17.2 | 0.3 | 0.1 | 0.7 | 0.1 | 14.9 | 0.6 | 2 |
| | | 34.4 | 0.2 | 0.0 | 0.8 | 0.0 | 24.1 | 0.0 | 1 |
| | | 68.8 | 0.2 | 0.0 | 0.4 | 0.0 | 35.9 | 0.0 | 1 |
| | | 137.5 | 0.2 | 0.0 | 0.5 | 0.0 | 48.6 | 2.1 | 2 |
| | | 275.0 | 0.1 | 0.0 | 0.4 | 0.0 | 63.8 | 0.0 | 1 |
| | | 550.0 | Not assayed | | | | | | |
| G018117 | BC22n | 0.0 | 0.3 | 0.0 | 2.7 | 0.1 | 0.3 | 0.0 | 2 |
| CIITA | | 8.6 | 14.5 | 1.5 | 3.8 | 0.3 | 3.5 | 0.3 | 2 |
| | | 17.2 | 28.1 | 0.6 | 3.5 | 0.3 | 3.9 | 1.0 | 2 |
| | | 34.4 | 45.9 | 0.4 | 3.3 | 0.4 | 3.6 | 0.0 | 2 |
| | | 68.8 | 62.8 | 5.3 | 3.6 | 0.1 | 3.7 | 1.2 | 2 |
| | | 137.5 | 78.9 | 1.3 | 2.7 | 0.1 | 2.7 | 0.7 | 2 |
| | | 275.0 | 86.3 | 1.8 | 2.6 | 0.1 | 2.0 | 0.1 | 2 |
| | | 550.0 | 92.3 | 1.2 | 2.6 | 0.2 | 1.1 | 0.2 | 2 |
| | Cas9 | 0.0 | 0.2 | 0.0 | 2.8 | 0.1 | 0.3 | 0.0 | 2 |
| | | 8.6 | 0.3 | 0.0 | 2.5 | 0.0 | 6.0 | 0.2 | 2 |
| | | 17.2 | 0.2 | 0.0 | 2.4 | 0.1 | 11.2 | 1.6 | 2 |
| | | 34.4 | 0.2 | 0.0 | 2.1 | 0.0 | 20.8 | 0.3 | 2 |
| | | 68.8 | 0.2 | 0.0 | 1.9 | 0.1 | 33.2 | 0.4 | 2 |
| | | 137.5 | 0.1 | 0.0 | 1.3 | 0.1 | 51.2 | 0.0 | 2 |
| | | 275.0 | 0.1 | 0.0 | 0.9 | 0.2 | 64.5 | 0.9 | 2 |
| | | 550.0 | 0.1 | 0.0 | 0.6 | 0.0 | 78.4 | 1.1 | 2 |
| G016086 | BC22n | 0.0 | 0.2 | 0.0 | 1.0 | 0.1 | 0.1 | 0.0 | 2 |
| CIITA | | 8.6 | 23.5 | 1.8 | 3.2 | 0.1 | 3.7 | 0.1 | 2 |
| | | 17.2 | 40.9 | 1.1 | 4.4 | 0.7 | 4.6 | 1.0 | 2 |
| | | 34.4 | 58.0 | 0.5 | 4.6 | 0.3 | 3.8 | 0.6 | 2 |
| | | 68.8 | 73.5 | 0.7 | 3.7 | 0.0 | 2.8 | 0.5 | 2 |
| | | 137.5 | 83.8 | 1.1 | 3.7 | 0.5 | 2.0 | 0.7 | 2 |
| | | 275.0 | 90.1 | 2.4 | 3.1 | 0.1 | 1.9 | 0.8 | 2 |
| | | 550.0 | 93.4 | 0.9 | 3.0 | 0.2 | 1.2 | 0.3 | 2 |
| | Cas9 | 0.0 | 0.2 | 0.0 | 1.0 | 0.1 | 0.1 | 0.0 | 2 |
| | | 8.6 | 0.2 | 0.0 | 1.1 | 0.2 | 7.4 | 0.7 | 2 |
| | | 17.2 | 0.2 | 0.0 | 1.1 | 0.3 | 17.7 | 1.0 | 2 |
| | | 34.4 | 0.2 | 0.0 | 0.8 | 0.1 | 32.1 | 0.1 | 2 |
| | | 68.8 | 0.2 | 0.0 | 0.7 | 0.2 | 51.5 | 0.8 | 2 |
| | | 137.5 | 0.2 | 0.0 | 0.4 | 0.0 | 69.3 | 0.1 | 2 |
| | | 275.0 | 0.3 | 0.1 | 0.3 | 0.1 | 84.2 | 0.1 | 2 |
| | | 550.0 | 0.3 | 0.0 | 0.1 | 0.1 | 90.0 | 0.7 | 2 |

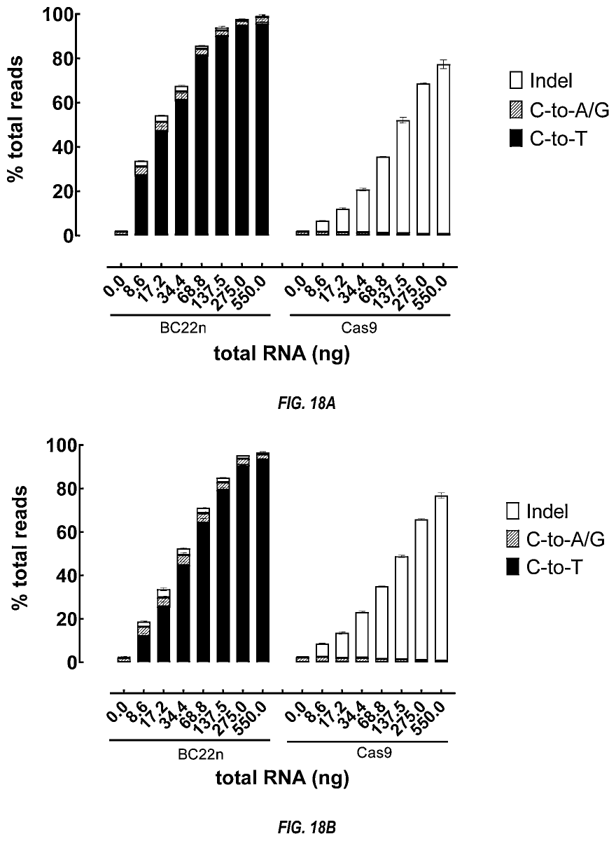

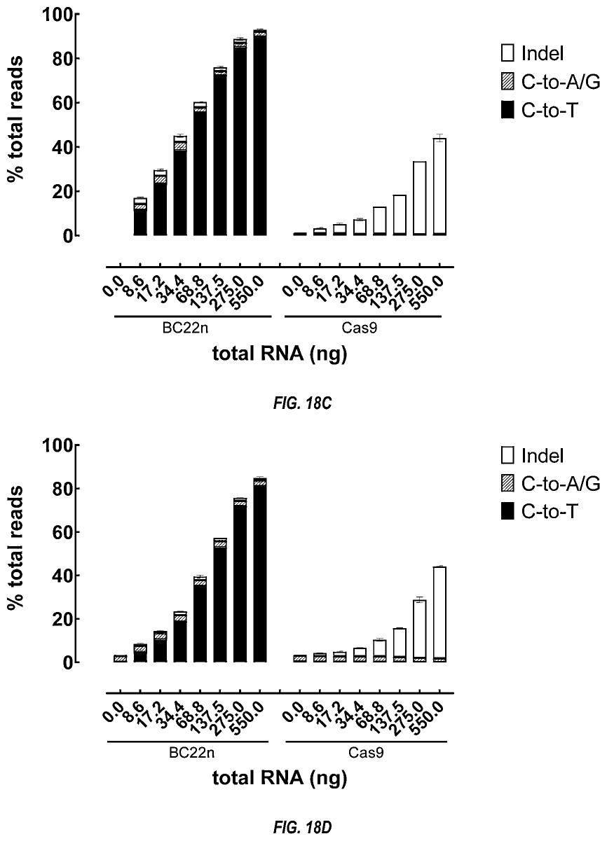

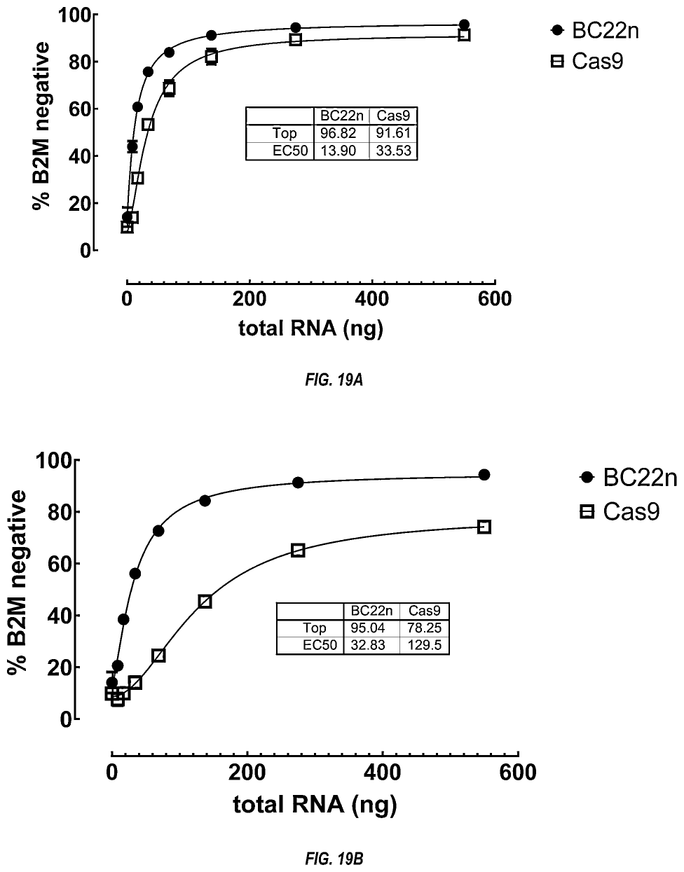

Table 27 and FIGS. 18A-18D describe the editing profile for T cells in percent of total reads when four guides were used simultaneously for editing. In this arm of the experiment, each guide is used at 25% the concentration compared to the single guide editing experiment. In total, T cells were exposed to 6 different LNPs simultaneously (editor mRNA, UGI mRNA, 4 guides). Editing with BC22n and trans UGI lead to higher percentages of maximum total editing for each locus compared to editing with Cas9.

TABLE 27
Editing as a percentage of total reads-multiple guide
delivery

| | | Total | % | | % | | % | | |
|---|---|---|---|---|---|---|---|---|---|
| Locus | | RNA | C-to-T | | C-to-A/G | | Indel | | |
| Assayed | Editor | (ng) | mean | SD | mean | SD | Mean | SD | N |
| G015995 | BC22n | 0.0 | 0.3 | 0.0 | 1.5 | 0.2 | 0.2 | 0.0 | 2 |
| B2M | | 8.6 | 27.3 | 0.2 | 3.8 | 0.1 | 2.6 | 0.1 | 2 |
| | | 17.2 | 47.2 | 2.2 | 4.1 | 0.4 | 3.0 | 0.1 | 2 |
| | | 34.4 | 61.2 | 3.0 | 3.9 | 0.1 | 2.6 | 0.3 | 2 |
| | | 68.8 | 81.4 | 0.1 | 2.9 | 0.1 | 1.4 | 0.1 | 2 |
| | | 137.5 | 90.0 | 1.1 | 2.6 | 0.3 | 1.3 | 0.5 | 2 |
| | | 275.0 | 94.7 | 0.1 | 2.2 | 0.1 | 0.8 | 0.0 | 2 |
| | | 550.0 | 95.9 | 0.9 | 2.9 | 1.0 | 0.4 | 0.3 | 2 |
| | Cas9 | 0.0 | 0.3 | 0.0 | 1.4 | 0.1 | 0.2 | 0.0 | 2 |
| | | 8.6 | 0.3 | 0.0 | 1.4 | 0.0 | 5.0 | 0.1 | 2 |
| | | 17.2 | 0.3 | 0.0 | 1.3 | 0.0 | 10.5 | 0.4 | 2 |
| | | 34.4 | 0.3 | 0.0 | 1.1 | 0.0 | 19.3 | 0.6 | 2 |
| | | 68.8 | 0.3 | 0.0 | 0.9 | 0.0 | 34.4 | 0.1 | 2 |
| | | 137.5 | 0.2 | 0.0 | 0.7 | 0.0 | 51.1 | 1.3 | 2 |
| | | 275.0 | 0.2 | 0.1 | 0.5 | 0.0 | 68.0 | 0.1 | 2 |
| | | 550.0 | 0.3 | 0.1 | 0.4 | 0.1 | 76.7 | 2.0 | 2 |
| G016017 | BC22n | 0.0 | 0.1 | 0.1 | 1.9 | 0.6 | 0.2 | 0.0 | 2 |
| TRAC | | 8.6 | 12.1 | 1.3 | 4.3 | 0.2 | 2.4 | 0.2 | 2 |
| | | 17.2 | 25.7 | 2.2 | 4.2 | 0.5 | 3.8 | 0.7 | 2 |
| | | 34.4 | 44.7 | 1.4 | 4.7 | 1.0 | 3.0 | 0.3 | 2 |
| | | 68.8 | 64.2 | 1.9 | 4.4 | 0.6 | 2.5 | 0.1 | 2 |
| | | 137.5 | 79.3 | 1.1 | 3.6 | 0.4 | 2.1 | 0.1 | 2 |
| | | 275.0 | 90.7 | 0.0 | 3.0 | 0.1 | 1.5 | 0.0 | 2 |
| | | 550.0 | 93.3 | 0.6 | 2.4 | 0.1 | 0.9 | 0.4 | 2 |
| | Cas9 | 0.0 | 0.1 | 0.1 | 2.1 | 0.2 | 0.1 | 0.0 | 2 |
| | | 8.6 | 0.2 | 0.1 | 2.3 | 0.2 | 6.1 | 0.2 | 2 |
| | | 17.2 | 0.1 | 0.0 | 1.8 | 0.2 | 11.5 | 0.5 | 2 |
| | | 34.4 | 0.1 | 0.0 | 2.0 | 0.4 | 21.0 | 0.4 | 2 |
| | | 68.8 | 0.1 | 0.0 | 1.4 | 0.0 | 33.5 | 0.1 | 2 |
| | | 137.5 | 0.1 | 0.0 | 1.2 | 0.1 | 47.5 | 0.5 | 2 |
| | | 275.0 | 0.1 | 0.0 | 0.9 | 0.1 | 64.8 | 0.2 | 2 |
| | | 550.0 | 0.1 | 0.0 | 0.6 | 0.1 | 76.1 | 1.3 | 2 |
| G016206 | BC22n | 0.0 | No data | | | | | | |
| TRBC1/2 | | 8.6 | 11.6 | 0.3 | 2.6 | 0.2 | 2.8 | 0.3 | 2 |
| | | 17.2 | 23.4 | 0.4 | 3.6 | 0.3 | 2.6 | 0.5 | 2 |
| | | 34.4 | 38.5 | 1.4 | 3.7 | 0.2 | 2.9 | 0.7 | 2 |
| | | 68.8 | 55.6 | 1.7 | 2.3 | 0.4 | 2.4 | 0.0 | 2 |
| | | 137.5 | 72.4 | 1.2 | 1.8 | 0.5 | 1.7 | 0.5 | 2 |
| | | 275.0 | 85.1 | 1.0 | 1.9 | 0.5 | 1.7 | 0.6 | 2 |
| | | 550.0 | 89.8 | 2.8 | 2.2 | 0.1 | 0.9 | 0.3 | 2 |
| | Cas9 | 0.0 | 0.2 | 0.0 | 0.6 | 0.0 | 0.1 | 0.0 | 1 |
| | | 8.6 | 0.2 | 0.1 | 0.7 | 0.1 | 2.3 | 0.3 | 2 |
| | | 17.2 | 0.3 | 0.0 | 0.7 | 0.3 | 4.2 | 0.4 | 2 |
| | | 34.4 | 0.1 | 0.0 | 0.5 | 0.1 | 6.6 | 0.5 | 2 |
| | | 68.8 | 0.4 | 0.0 | 0.5 | 0.0 | 12.3 | 0.0 | 1 |
| | | 137.5 | 0.2 | 0.0 | 0.5 | 0.0 | 17.8 | 0.0 | 1 |
| | | 275.0 | 0.1 | 0.0 | 0.5 | 0.0 | 33.0 | 0.0 | 1 |
| | | 550.0 | 0.3 | 0.2 | 0.3 | 0.0 | 43.3 | 1.7 | 2 |
| G018117 | BC22n | 0.0 | 0.2 | 0.0 | 2.6 | 0.1 | 0.3 | 0.0 | 2 |
| CIITA | | 8.6 | 4.6 | 0.9 | 3.1 | 0.2 | 0.8 | 0.2 | 2 |
| | | 17.2 | 10.5 | 0.2 | 2.9 | 0.1 | 1.1 | 0.2 | 2 |
| | | 34.4 | 18.8 | 0.3 | 2.9 | 0.2 | 1.6 | 0.2 | 2 |
| | | 68.8 | 35.1 | 0.6 | 2.7 | 0.2 | 1.6 | 0.7 | 2 |
| | | 137.5 | 52.9 | 0.2 | 2.9 | 0.3 | 1.5 | 0.0 | 2 |
| | | 275.0 | 71.9 | 2.4 | 2.5 | 0.3 | 1.3 | 0.1 | 2 |
| | | 550.0 | 81.1 | 1.9 | 2.6 | 0.1 | 1.1 | 0.6 | 2 |
| | Cas9 | 0.0 | 0.3 | 0.0 | 2.7 | 0.1 | 0.3 | 0.0 | 2 |
| | | 8.6 | 0.2 | 0.0 | 2.6 | 0.2 | 1.4 | 0.0 | 2 |
| | | 17.2 | 0.2 | 0.0 | 2.5 | 0.0 | 2.1 | 0.3 | 2 |
| | | 34.4 | 0.3 | 0.0 | 2.5 | 0.0 | 3.9 | 0.1 | 2 |
| | | 68.8 | 0.2 | 0.0 | 2.5 | 0.2 | 7.7 | 0.6 | 2 |
| | | 137.5 | 0.2 | 0.0 | 2.2 | 0.1 | 13.3 | 0.2 | 2 |
| | | 275.0 | 0.1 | 0.0 | 1.9 | 0.0 | 26.7 | 1.3 | 2 |
| | | 550.0 | 0.1 | 0.0 | 1.7 | 0.1 | 42.3 | 0.3 | 2 |

On day 10 post-activation, T cells were phenotyped by flow cytometry to determine if editing resulted in loss of cell surface proteins. Briefly, T cells were incubated in a mix of the following antibodies: B2M-FITC (BioLegend, Cat. 316304), CD3-AF700 (BioLegend, Cat. 317322), HLA DR DQ DP-PE (BioLegend, Cat 361704) and DAPI (BioLegend, Cat 422801). A subset of unedited cells was incubated with Isotype Control-PE (BioLegend® Cat. No. 400234). Cells were subsequently washed, processed on a Cytoflex instrument (Beckman Coulter), and analyzed using the FlowJo software package. T cells were gated based on size, shape, viability, and antigen expression.

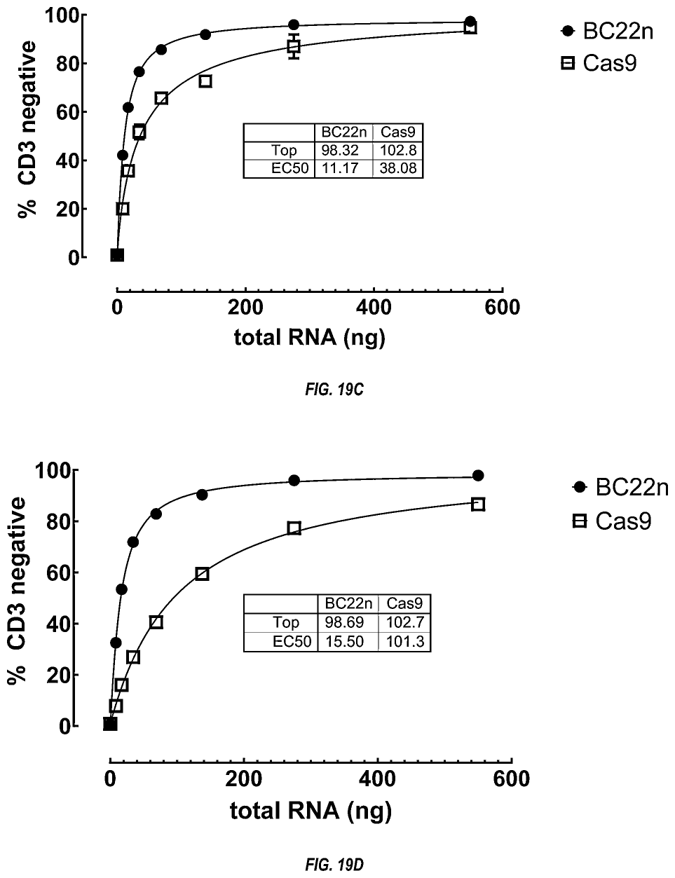

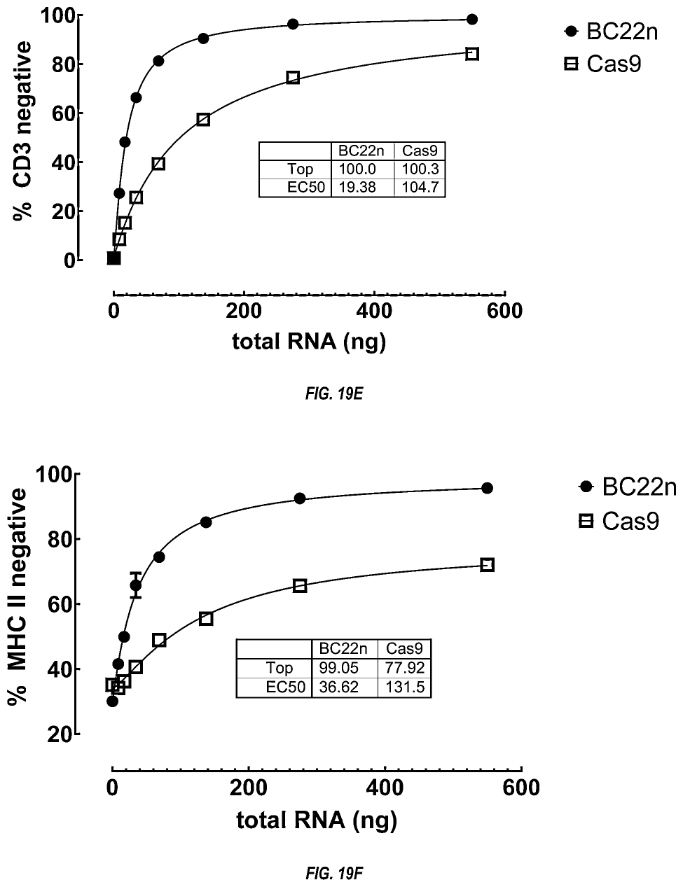

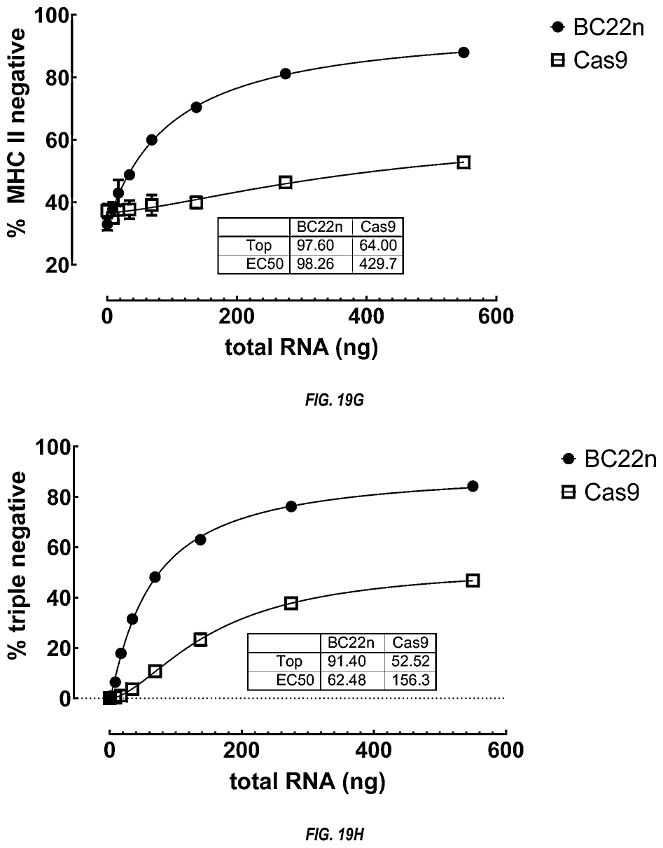

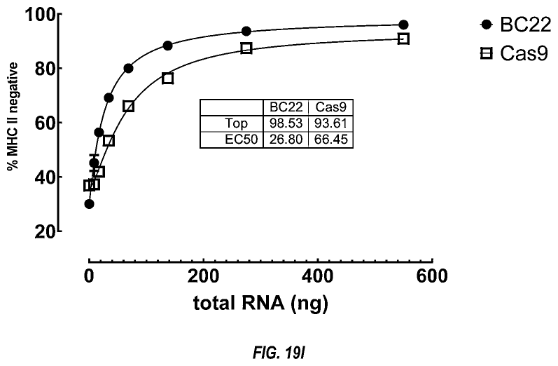

Table 28 and FIGS. 19A-19I report phenotyping results as percent of cells negative for antibody binding. The percentage of antigen negative cells increased in a dose responsive manner with increasing total RNA for both BC22n and Cas9 samples. Cells edited with BC22n showed comparable or higher protein knockout compared to cells edited with Cas9 for all guides tested. In multi-edited cells, BC22n with trans UGI showed substantially higher percentages of antigen negative cells than Cas9 with trans UGI. For example, BC22n edited samples at the highest total RNA dose of 550 ng showed 84.2% of cells lacking all three antigens, while Cas9 editing led to only 46.8% such triple knockout cells. For samples treated with one guide only, the correlation between DNA editing and antigen reduction was robust. BC22n had an R square measurement of 0.93 when comparing C to T conversions to antigen knockout. Cas9 had an R square measurement of 0.95 when comparing indels to antigen knockout.

TABLE 28
Flow cytometry data-percent cells negative for antigen
(n = 2)

| | | Total | BC22n | | Cas9 | |
|---|---|---|---|---|---|---|
| | | RNA | Mean | | Mean | |
| Guide(s) | Phenotype | (ng) | % | SD | % | SD |
| G015995 | B2M neg | 550.0 | 95.7 | 0.1 | 91.3 | 0.6 |
| B2M | | 275.0 | 94.4 | 0.4 | 89.3 | 0.1 |
| | | 137.5 | 91.2 | 0.1 | 82.1 | 3.3 |
| | | 68.8 | 83.9 | 0.4 | 68.7 | 3.3 |
| | | 34.4 | 75.7 | 1.4 | 53.4 | 0.2 |
| | | 17.2 | 60.8 | 2.0 | 30.7 | 1.3 |
| | | 8.6 | 44.0 | 2.3 | 13.9 | 2.0 |
| | | 0.0 | 14.1 | 4.1 | 9.9 | 1.9 |
| G015995 | B2M neg | 550.0 | 94.4 | 0.1 | 74.2 | 0.4 |
| G016017 | | 275.0 | 91.3 | 0.1 | 65.2 | 0.1 |
| G016206 | | 137.5 | 84.3 | 0.2 | 45.4 | 1.9 |
| G018117 | | 68.8 | 72.7 | 0.4 | 24.5 | 0.8 |
| | | 34.4 | 56.2 | 1.2 | 14.1 | 2.3 |
| | | 17.2 | 38.5 | 0.2 | 9.9 | 0.8 |
| | | 8.6 | 20.6 | 0.7 | 7.6 | 2.4 |
| | | 0.0 | 14.1 | 4.1 | 9.9 | 1.9 |
| G016017 | CD3 neg | 550.0 | 97.3 | 0.3 | 94.8 | 0.4 |
| TRAC | | 275.0 | 96.0 | 0.2 | 87.0 | 4.9 |
| | | 137.5 | 91.9 | 0.2 | 72.7 | 0.9 |
| | | 68.8 | 85.7 | 0.5 | 65.6 | 0.1 |
| | | 34.4 | 76.6 | 0.8 | 51.7 | 3.0 |
| | | 17.2 | 61.8 | 1.7 | 35.7 | 1.1 |
| | | 8.6 | 42.1 | 0.7 | 20.1 | 1.5 |
| | | 0.0 | 1.0 | 0.1 | 0.9 | 0.1 |
| G016206 | CD3 neg | 550.0 | 97.9 | 0.1 | 86.6 | 0.3 |
| TRBC1/2 | | 275.0 | 96.0 | 0.1 | 77.3 | 0.1 |
| | | 137.5 | 90.4 | 0.8 | 59.4 | 0.4 |
| | | 68.8 | 82.9 | 0.1 | 40.6 | 1.2 |
| | | 34.4 | 71.9 | 1.5 | 27.0 | 1.6 |
| | | 17.2 | 53.4 | 0.3 | 16.1 | 0.1 |
| | | 8.6 | 32.6 | 0.6 | 7.9 | 0.4 |
| | | 0.0 | 0.8 | 0.0 | 0.9 | 0.4 |
| G015995 | CD3 neg | 550.0 | 98.3 | 0.2 | 84.2 | 0.1 |
| G016017 | | 275.0 | 96.3 | 0.1 | 74.6 | 0.5 |
| G016206 | | 137.5 | 90.4 | 0.3 | 57.4 | 1.0 |
| G018117 | | 68.8 | 81.3 | 0.3 | 39.4 | 0.1 |
| | | 34.4 | 66.3 | 1.6 | 25.6 | 0.8 |
| | | 17.2 | 48.2 | 1.0 | 15.3 | 0.5 |
| | | 8.6 | 27.3 | 0.7 | 8.6 | 0.5 |
| | | 0.0 | 0.9 | 0.1 | 0.9 | 0.2 |
| G018117 | MHC II | 550.0 | 95.7 | 0.4 | 72.0 | 0.1 |
| CIITA | neg | 275.0 | 92.5 | 1.1 | 65.6 | 0.4 |
| | | 137.5 | 85.2 | 0.6 | 55.5 | 0.6 |
| | | 68.8 | 74.5 | 1.1 | 48.9 | 0.0 |
| | | 34.4 | 65.8 | 3.7 | 40.7 | 0.6 |
| | | 17.2 | 49.9 | 0.1 | 36.2 | 0.6 |
| | | 8.6 | 41.6 | 0.8 | 34.2 | 1.3 |
| | | 0.0 | 30.1 | 1.6 | 35.2 | 0.4 |
| G015995 | MHC II | 550.0 | 88.0 | 0.2 | 52.8 | 1.1 |
| G016017 | neg | 275.0 | 81.2 | 0.2 | 46.4 | 0.4 |
| G016206 | | 137.5 | 70.4 | 1.3 | 39.9 | 1.8 |
| G018117 | | 68.8 | 60.0 | 0.4 | 39.1 | 3.3 |
| | | 34.4 | 48.8 | 0.6 | 37.7 | 2.9 |
| | | 17.2 | 43.0 | 4.2 | 37.5 | 0.6 |
| | | 8.6 | 37.8 | 2.1 | 35.0 | 0.0 |
| | | 0.0 | 33.0 | 1.9 | 37.3 | 2.1 |
| G015995 | B2M neg | 550.0 | 84.2 | 0.0 | 46.8 | 1.1 |
| G016017 | CD3 neg | 275.0 | 76.2 | 0.0 | 37.8 | 0.2 |
| G016206 | MHC II | 137.5 | 63.0 | 1.3 | 23.4 | 2.4 |
| G018117 | neg | 68.8 | 48.2 | 0.2 | 10.8 | 0.9 |
| | | 34.4 | 31.5 | 1.1 | 3.6 | 0.9 |
| | | 17.2 | 17.8 | 1.7 | 1.1 | 0.2 |
| | | 8.6 | 6.4 | 0.0 | 0.4 | 0.1 |
| | | 0.0 | 0.1 | 0.0 | 0.1 | 0.0 |
| G016086 | HLA DR | 550.0 | 96.0 | 0.1 | 90.9 | 0.7 |
| CIITA | DP DQ | 275.0 | 93.7 | 0.1 | 87.4 | 0.3 |
| | neg | 137.5 | 88.4 | 0.5 | 76.3 | 0.6 |
| | | 68.8 | 80.0 | 0.7 | 66.1 | 1.8 |
| | | 34.4 | 69.2 | 1.5 | 53.4 | 1.1 |
| | | 17.2 | 56.4 | 0.4 | 41.9 | 0.8 |
| | | 8.6 | 45.2 | 2.9 | 37.3 | 0.1 |
| | | 0.0 | 30.1 | 0.9 | 36.8 | 0.4 |

### Example 9—Editing in T Cells with Trans UGI Titration

Editing profiles were assessed to determine the concentration of UGI mRNA necessary to achieve highly pure C-to-T editing in T cells with different editing constructs. C-to-T editing purity (% of edited reads containing only C-to-T conversions) was measured using a saturating dose of editor mRNA and sgRNA and varying amounts of UGI mRNA. Editor mRNAs included an mRNA encoding BC22n (SEQ ID NO: 5), an mRNA encoding BC22 with a total of 2 fused UGI moieties (SEQ ID NO: 29), and an mRNA encoding BE4Max which includes 2 fused UGI moieties (SEQ ID NO: 32) (Koblan L W, Doman J L, Wilson C, et al. Nat Biotechnol. 2018; 36(9):843-846).

Solutions containing editor mRNA as listed in Table 29 or UGI mRNA (SEQ ID NO: 26) were prepared in sterile water. 50 μM B2M targeting sgRNA G015995 were removed from their storage plates and denatured for 2 minutes at 95° C. before cooling on ice. Seventy-two hours post activation, T cells were harvested, centrifuged, and resuspended at a concentration of 12.5×10\hat{ }6 T cells/mL in P3 electroporation buffer (Lonza). For each well to be electroporated, 1×10\hat{ }5 T cells were mixed with 200 ng of editor mRNAs, 20 pmols of sgRNA and different concentrations of UGI mRNA ranging from 0.8 ng to 600.0 ng (0.8 nM to 597.0 nM), in a final volume of 20 uL of P3 electroporation buffer. This mRNA+sgRNA+T cell mix was transferred in triplicate to a 96-well Nucleofector™ plate and electroporated using the manufacturer's pulse code. Electroporated T cells were immediately rested in CTS™ OpTmizer™ T Cell Expansion serum-free media (SFM) (ThermoFisher Cat. A1048501) without cytokines for 10 minutes before being transferred to a new flat-bottom 96-well plate containing an additional 100 uL of the same media with 2× concentration of cytokines. The resulting plate was incubated at 37° C. for 9 days, during which time the cells were split and media refreshed two times. T cells were collected and processed for NGS sequencing as described in Example 1.

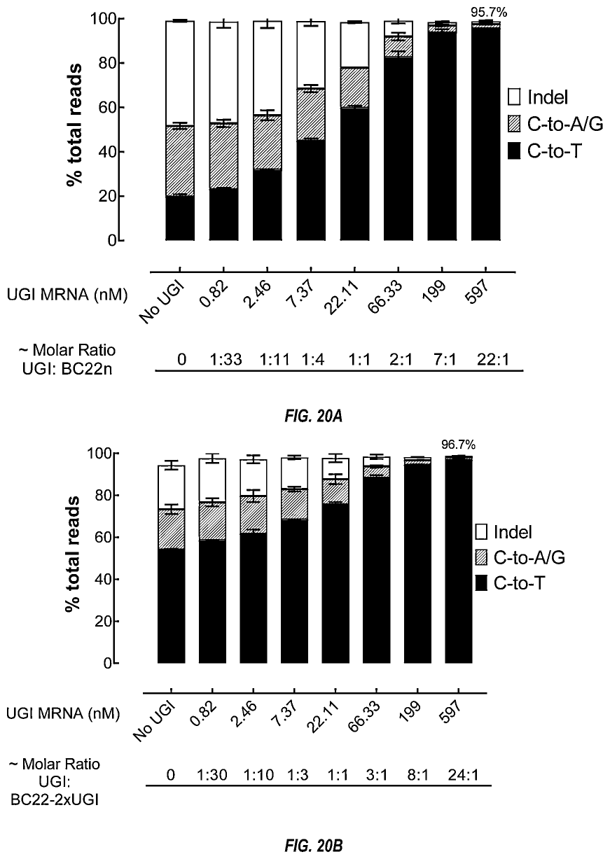

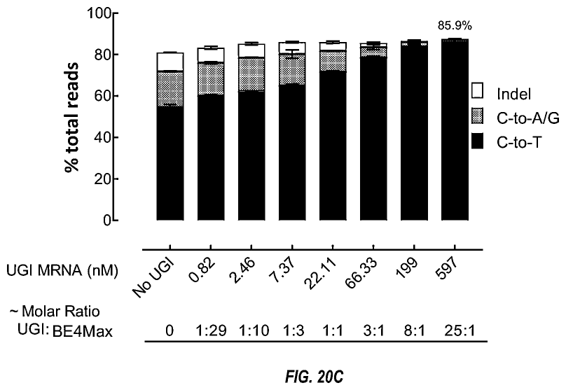

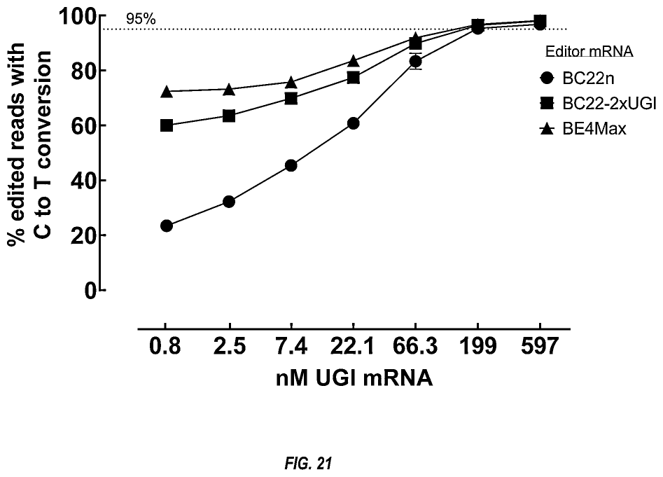

Table 29 and FIGS. 20A-20C show the percentage of reads with each edit type. Increasing levels of UGI mRNA in trans decreased indels for all three editor constructs. With 199 nM UGI mRNA or higher, indels were decreased to less than 2% of total reads for each editor construct tested. At low concentrations of UGI mRNA in trans, editor constructs that encoded UGI showed more C to T conversions than the BC22n construct that lacked an encoded UGI. Table 30 and FIG. 21 represents the data captured in Table 29 and FIGS. 20A-20C as percentage of total edits that were C to T conversions, also known as the C to T purity. Increasing UGI mRNA in trans increased C to T total editing and increased C to T purity for all constructs. All three constructs showed over 95% C to T editing purity with 199 nM UGI mRNA, which corresponds to a molar ratio of UGI mRNA to editor mRNA of about 7:1.

TABLE 29
Editing type as a percentage of total reads with increasing
UGI mRNA

| | nM UGI | % C-to-T | | % C-to-A/G | | % Indel | |
|---|---|---|---|---|---|---|---|
| Editor | mRNA | mean | SD | mean | SD | mean | SD |
| BC22n | No UGI | 19.9 | 1.1 | 31.8 | 1.4 | 47.4 | 0.4 |
| | 0.82 | 23.2 | 0.6 | 29.7 | 1.6 | 45.9 | 2.8 |
| | 2.46 | 32.0 | 0.2 | 24.5 | 2.3 | 42.6 | 3.3 |
| | 7.37 | 45.0 | 1.1 | 23.6 | 1.6 | 30.4 | 2.1 |
| | 22.11 | 59.8 | 1.0 | 18.1 | 0.1 | 20.6 | 0.4 |
| | 66.33 | 82.7 | 2.6 | 9.3 | 1.7 | 7.2 | 1.2 |
| | 199 | 93.8 | 1.4 | 3.2 | 0.4 | 1.5 | 0.3 |
| | 597 | 95.7 | 0.1 | 1.9 | 0.2 | 1.3 | 0.4 |
| BC22- | No UGI | 54.2 | 0.4 | 19.2 | 2.3 | 21.0 | 2.1 |
| 2xUGI | 0.82 | 58.6 | 0.1 | 18.1 | 1.9 | 21.0 | 2.3 |
| | 2.46 | 61.7 | 2.0 | 18.0 | 2.9 | 17.5 | 1.8 |
| | 7.37 | 68.5 | 0.1 | 14.5 | 1.1 | 15.1 | 0.8 |
| | 22.11 | 75.7 | 1.1 | 12.1 | 2.3 | 10.1 | 2.1 |
| | 66.33 | 88.4 | 1.2 | 5.4 | 0.6 | 4.7 | 1.1 |
| | 199 | 94.6 | 0.1 | 2.1 | 0.1 | 1.5 | 0.1 |
| | 597 | 96.9 | 0.6 | 1.4 | 0.1 | 0.6 | 0.1 |
| BE4Max | No UGI | 54.7 | 1.3 | 17.2 | 0.3 | 9.1 | 0.1 |
| | 0.82 | 60.3 | 0.5 | 15.8 | 0.5 | 7.3 | 0.7 |
| | 2.46 | 62.4 | 0.1 | 16.2 | 0.2 | 6.7 | 0.6 |
| | 7.37 | 65.2 | 0.6 | 15.1 | 2.0 | 5.8 | 0.4 |
| | 22.11 | 71.8 | 0.4 | 10.0 | 0.2 | 4.2 | 0.6 |
| | 66.33 | 78.6 | 0.6 | 4.9 | 1.1 | 2.1 | 0.4 |
| | 199 | 83.8 | 1.0 | 2.3 | 0.8 | 0.5 | 0.1 |
| | 597 | 85.9 | 1.6 | 1.2 | 0.1 | 0.4 | 0.1 |

TABLE 30
C-to-T purity: Percent of edited sequencing reads containing only
C-to-T conversions, no indels, no C to A/G conversions (n = 2)

| nM UGI | BC22n | | BC22-2XUGI | | BE4Max | |
|---|---|---|---|---|---|---|
| mRNA | mean | SD | mean | SD | mean | SD |
| 597 | 96.8 | 0.7 | 98.0 | 0.1 | 98.2 | 0.1 |
| 199 | 95.3 | 0.7 | 96.4 | 0.1 | 96.8 | 1.1 |
| 66.33 | 83.4 | 2.9 | 89.8 | 1.6 | 91.9 | 0.9 |
| 22.11 | 60.8 | 0.6 | 77.4 | 0.4 | 83.6 | 0.8 |
| 7.37 | 45.4 | 0.8 | 69.9 | 0.2 | 75.8 | 1.6 |
| 2.46 | 32.3 | 0.5 | 63.5 | 1.4 | 73.2 | 0.7 |
| 0.82 | 23.5 | 0.8 | 60.0 | 0.1 | 72.4 | 0.1 |
| 0 | 19.1 | 0.4 | 57.4 | 0.1 | 67.5 | 0.8 |

### Example 10. Screening of CIITA Guide RNAs

CIITA gRNAs were screened for efficacy in T cells by assessing knockdown of MHC class II cell surface expression using both Cas9 and BC22. The percentage of T cells negative for MHC class II protein was assayed following CIITA editing by electroporation with RNP.

### 10.1. RNP Electroporation of T Cells

Cas9 editing activity was assessed using electroporation of Cas9 ribonucleoprotein (RNP). Upon thaw, Pan CD3+ T cells (StemCell, HLA-A*02.01/A*03.01) were plated at a density of 0.5×10\hat{ }6 cells/mL in T cell RPMI media composed of RPMI 1640 (Invitrogen, Cat. 22400-089) containing 5% (v/v) of fetal bovine serum, 1× Glutamax (Gibco, Cat. 35050-061), 50 μM of 2-Mercaptoethanol, 100 uM non-essential amino acids (Invitrogen, Cat. 11140-050), 1 mM sodium pyruvate, 10 mM HEPES buffer, 1% of Penicillin-Streptomycin, and 100 U/mL of recombinant human interleukin-2 (Peprotech, Cat. 200-02). T cells were activated with TransAct™ (1:100 dilution, Miltenyi Biotec). Cells were expanded in T cell RPMI media for 72 hours prior to RNP transfection.

CIITA targeting sgRNAs were removed from their storage plates and denatured for 2 minutes at 95° C. before cooling at room temperature for 10 minutes. RNP mixture of 20 uM sgRNA and 10 uM recombinant Cas9-NLS protein (SEQ ID No. 36) was prepared and incubated at 25° C. for 10 minutes. Five μL of RNP mixture was combined with 100,000 cells in 20 μL P3 electroporation Buffer (Lonza). 22 μL of RNP/cell mix was transferred to the corresponding wells of a Lonza shuttle 96-well electroporation plate. Cells were electroporated in duplicate with the manufacturer's pulse code. T cell RPMI media was added to the cells immediately post electroporation. Electroporated T cells were subsequently cultured and collected for NGS sequencing as described in Example 1 at 2 days post edit.

### 10.2 Flow Cytometry

On day 10 post-edit, T cells were phenotyped by flow cytometry to determine HLA class II protein expression. Briefly, T cells were incubated in cocktails of antibodies targeting HLA-DR, DQ, DP-PE (BioLegend® Cat. No. 361704) and Isotype Control-AF647 (BioLegend® Cat. No. 400234). Cells were subsequently washed, processed on a Cytoflex flow cytometer (Beckman Coulter) and analyzed using the FlowJo software package. T cells were gated based on size, shape, viability, and MHC II expression. DNA samples were subjected to PCR and subsequent NGS analysis, as described in Example 1.

10.3. mRNA Electroporation of T Cells

BC22 editing activity was assayed following CIITA editing by electroporation with mRNA and guide. Upon thaw, Pan CD3+ T cells isolated from a commercially obtained leukopak (StemCell) were plated at a density of 0.5×10\hat{ }6 cells/mL in T cell R10 media composed of RPMI 1640 (Invitrogen, Cat. 22400-089) containing 10% (v/v) of fetal bovine serum, 2 mM Glutamax (Gibco, Cat. 35050-061), 22 μM of 2-Mercaptoethanol, 100 uM non-essential amino acids (Invitrogen, Cat. 11140-050), 1 mM sodium pyruvate, 10 mM HEPES buffer, 1% of Penicillin-Streptomycin, plus 100 U/mL of recombinant human interleukin-2 (Peprotech, Cat. 200-02). T cells were activated with Dynabeads® Human T-Activator CD3/CD28 (ThermoFisher). Cells were expanded in T cell for 72 hours prior to mRNA transfection.

CIITA sgRNAs were removed from their storage plates and denatured for 2 minutes at 95° C. before cooling at room temperature for 10 minutes. Fifty microliter electroporation mix was prepared with 100,00 T cells in P3 buffer (Lonza) and 10 ng/uL mRNA encoding UGI (SEQ ID NO: 26), 10 ng/uL mRNA encoding BC22 (SEQ ID NO: 20) and 2 uM sgRNA. This mix was transferred to the corresponding wells of a Lonza shuttle 96-well electroporation plate. Cells were electroporated in duplicate wells using Lonza shuttle 96 w program with the manufacturer's pulse code. R10 media with IL-2 was added to the cells immediately post electroporation. Electroporated T cells were subsequently cultured and collected for NGS sequencing and flow cytometry 10 days post edit. Flow cytometry was performed as described for Cas9 RNP treated cells above. DNA samples were subjected to PCR and subsequent NGS analysis, as described in Example 1.

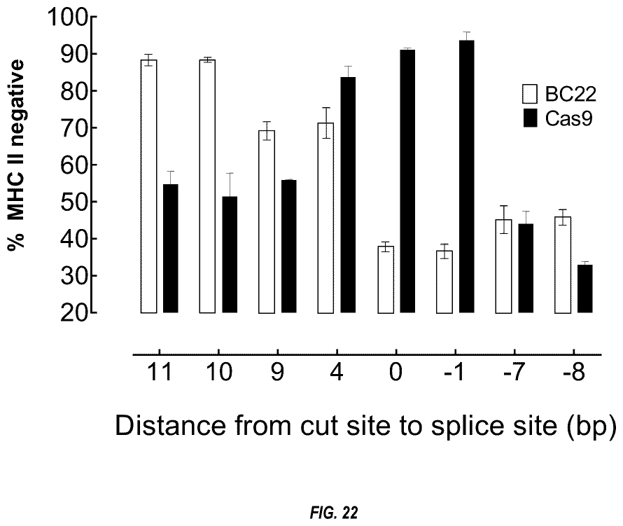

Table 31 and FIG. 22 show mean percentage of T cells negative for cell surface expression of MHC Class II proteins HLA-DR, DQ, DP using Cas9 and BC22 in relation to the distance from the cut site to the splice site boundary nucleotide. For each guide, the genomic coordinate of the cut site with SpCas9 is shown, as well as the distance (# of nucleotides) between the acceptor splice site boundary nucleotide or the donor splice site boundary nucleotide and the cut site. Positive numerical values show the number of nucleotides in the 5′directed between a splice site boundary nucleotide and cut site, whereas the negative numerical values show the number of nucleotides in the 3′ direction between a splice site boundary nucleotide and cut site.

TABLE 31
Guide position vs. protein knockdown efficiency

| | Distance | | | | |
|---|---|---|---|---|---|
| | from Cut | BC22 | | Cas9 | |
| | Site | Mean % MHC | | Mean % MHC | |
| Guide | (bp) | II negative | SD | II negative | SD |
| G018075 | 11 | 88.3 | 1.56 | 54.7 | 3.54 |
| G018076 | 10 | 88.45 | 0.64 | 51.45 | 6.29 |
| G018077 | 9 | 69.15 | 2.48 | 55.9 | 0.14 |
| G018078 | 4 | 71.35 | 4.17 | 83.65 | 3.04 |
| G018081 | 0 | 37.9 | 1.27 | 91.1 | 0.42 |
| G018082 | −1 | 36.75 | 1.91 | 93.6 | 2.26 |
| G018084 | −7 | 45.25 | 3.75 | 44 | 3.39 |
| G018085 | −8 | 45.75 | 2.19 | 32.95 | 0.92 |

### Example 11—Screening of HLA-A Guides with BC22n and Cas9

HLA-A guide RNAs were screened for efficacy in T cells by assessing loss of HLA-A cell surface expression. The percentage of T cells negative for HLA-A protein in an HLA-A2 background (“% HLA-A2-”) was assayed by flow cytometry following HLA-A editing by mRNA delivery.

11.1. mRNA Electroporation of T Cells

Cas9 and BC22n editing activity was assessed using electroporation of mRNA encoding Cas9 (SEQ ID NO: 11), mRNA encoding BC22n (SEQ ID NO: 2), or mRNA encoding UGI (SEQ ID NO: 26), as provided below. Upon thaw, Pan CD3+ T cells (StemCell, HLA-A*02.01/A*02.01) were plated at a density of 1×10\hat{ }6 cells/mL in TCGM composed of CTS OpTmizer T Cell Expansion SFM (Thermofisher, Cat. A3705001) supplemented with 5% human AB serum (Gemini, Cat. 100-512), 1× GlutaMAX (Thermofisher, Cat. 35050061), 10 mM HEPES (Thermofisher, Cat. 15630080), 1× of Penicillin-Streptomycin, further supplemented with 200 U/mL IL-2 (Peprotech, Cat. 200-02), 10 ng/ml IL-7 (Peprotech, Cat. 200-07), 10 ng/ml IL-15 (Peprotech, Cat. 200-15). T cells were activated with TransAct™ (1:100 dilution, Miltenyi Biotec). Cells were expanded in T cell RPMI media for 72 hours at 37° C. prior to mRNA electroporation.

HLA-A sgRNAs were removed from their storage plates and denatured for 2 minutes at 95° C. before incubating at room temperature for 5 minutes. BC22n electroporation mix was prepared with 100,000 T cells in P3 buffer (Lonza), 200 ng of mRNA encoding UGI, 200 ng of mRNA encoding BC22n and 20 pmoles of sgRNA. Cas9 electroporation mix was prepared with 100,000 T cells in P3 buffer (Lonza), 200 ng of mRNA encoding UGI, 200 ng of mRNA encoding Cas9 and 20 pmoles of sgRNA. Each mix was transferred to the corresponding wells of a Lonza shuttle 96-well electroporation plate. Cells were electroporated in duplicate using Lonza shuttle 96 w using manufacturer's pulse code. Immediately post electroporation, cells were recovered in pre-warmed TCGM without cytokines and incubated at 37° C. for 15 minutes. Electroporated T cells were subsequently cultured in TCGM with further supplemented with 200 U/mL IL-2 (Peprotech, Cat. 200-02), 10 ng/ml IL-7 (Peprotech, Cat. 200-07), 10 ng/ml IL-15 (Peprotech, Cat. 200-15) and collected for flow cytometry 8 days post edit.

### 11.2. Flow Cytometry

On day 8 post-edit, T cells were phenotyped by flow cytometry to determine HLA-A protein expression. Briefly, T cells were incubated with antibodies targeting HLA-A2, (eBioscience Cat. No. 17-9876-42). Cells were subsequently washed, processed on a Cytoflex flow cytometer (Beckman Coulter) and analyzed using the FlowJo software package. T cells were gated based on size, shape, viability, and HLA-A2 expression. Table 32 shows the percentage of cells negative for HLA-A surface proteins following genomic editing of HLA-A with BC22n or Cas9.

TABLE 32
Percentage of cells negative for HLA-A surface protein
following genomic editing of HLA-A with BC22n or Cas9.

| | BC22n | | Cas9 | |
|---|---|---|---|---|
| | Mean | SD % | Mean | SD % |
| Guide ID | % A2- | A2- | % A2- | A2- |
| G018932 | 20.15 | 2.76 | 43.30 | 1.70 |
| G018933 | 10.35 | 1.20 | 74.00 | 0.57 |
| G018934 | 0.50 | 0.14 | 15.30 | 1.56 |
| G018935 | 0.00 | 0.00 | 69.30 | 0.28 |
| G018936 | 0.10 | 0.00 | 29.65 | 2.62 |
| G018937 | 0.15 | 0.07 | 50.50 | 0.71 |
| G018938 | 0.00 | 0.00 | 0.00 | 0.00 |
| G018939 | 0.00 | 0.00 | 44.90 | 1.27 |
| G018940 | 0.00 | 0.00 | 12.00 | 0.42 |
| G018941 | 0.00 | 0.00 | 2.65 | 0.35 |
| G018942 | 0.10 | 0.00 | 2.15 | 0.07 |
| G018943 | 0.00 | 0.00 | 16.20 | 0.42 |
| G018944 | 0.00 | 0.00 | 3.00 | 0.28 |
| G018945 | 0.05 | 0.07 | 3.20 | 0.42 |
| G018946 | 0.00 | 0.00 | 2.30 | 0.14 |
| G018947 | 0.00 | 0.00 | 1.55 | 0.49 |
| G018949 | 0.00 | 0.00 | 47.10 | 0.57 |
| G018950 | 0.00 | 0.00 | 0.30 | 0.00 |
| G018951 | 0.00 | 0.00 | 13.30 | 0.28 |
| G018952 | 0.00 | 0.00 | 0.50 | 0.00 |
| G018953 | 0.00 | 0.00 | 3.65 | 0.64 |
| G018955 | 0.20 | 0.14 | 5.20 | 0.28 |
| G018958 | 0.00 | 0.00 | 1.30 | 0.28 |
| G018959 | 0.00 | 0.00 | 3.70 | 0.14 |
| G018960 | 0.00 | 0.00 | 0.35 | 0.07 |
| G018961 | 0.00 | 0.00 | 0.40 | 0.00 |
| G018962 | 0.00 | 0.00 | 2.90 | 0.42 |
| G018963 | 0.00 | 0.00 | 12.50 | 0.14 |
| G018964 | 0.00 | 0.00 | 6.45 | 0.64 |
| G018965 | 0.00 | 0.00 | 0.90 | 0.00 |
| G018966 | 0.00 | 0.00 | 1.30 | 0.14 |
| G018968 | 0.10 | 0.00 | 0.10 | 0.00 |
| G018969 | 0.00 | 0.00 | 0.80 | 0.14 |
| G018970 | 0.00 | 0.00 | 0.95 | 0.07 |
| G018971 | 0.00 | 0.00 | 0.10 | 0.00 |
| G018972 | 0.05 | 0.07 | 3.40 | 0.28 |
| G018973 | 0.00 | 0.00 | 1.35 | 0.07 |
| G018974 | 0.00 | 0.00 | 0.45 | 0.07 |
| G018976 | 0.05 | 0.07 | 2.45 | 0.07 |
| G018977 | 0.00 | 0.00 | 12.45 | 1.06 |
| G018978 | 0.00 | 0.00 | 1.75 | 0.07 |
| G018979 | 0.05 | 0.07 | 37.40 | 0.71 |
| G018980 | 0.05 | 0.07 | 32.40 | 2.40 |
| G018981 | 0.00 | 0.00 | 17.45 | 0.35 |
| G018982 | 0.00 | 0.00 | 26.35 | 0.92 |
| G018983 | 0.00 | 0.00 | 0.25 | 0.07 |
| G018984 | 0.00 | 0.00 | 0.65 | 0.07 |
| G018986 | 0.00 | 0.00 | 1.85 | 0.21 |
| G018987 | 0.00 | 0.00 | 2.25 | 0.07 |
| G018988 | 0.00 | 0.00 | 0.15 | 0.07 |
| G018989 | 0.00 | 0.00 | 1.85 | 0.07 |
| G018990 | 0.25 | 0.07 | 17.45 | 1.06 |
| G018991 | 0.20 | 0.00 | 23.15 | 0.92 |
| G018992 | 0.20 | 0.14 | 38.15 | 0.07 |
| G018993 | 0.15 | 0.07 | 12.15 | 1.34 |
| G018994 | 4.35 | 0.35 | 23.75 | 0.49 |
| G018995 | 0.55 | 0.07 | 94.27 | 0.30 |
| G018996 | 0.85 | 0.07 | 92.39 | 0.83 |
| G018997 | 97.80 | 0.08 | 95.03 | 1.87 |
| G018998 | 74.75 | 7.71 | 93.33 | 0.18 |
| G018999 | 98.26 | 0.30 | 96.05 | 2.27 |
| G019000 | 9.05 | 0.35 | 94.67 | 0.74 |
| G019001 | 0.05 | 0.07 | 4.05 | 0.64 |
| G019002 | 0.00 | 0.00 | 0.05 | 0.07 |
| G019003 | 0.00 | 0.00 | 11.10 | 0.00 |
| G019004 | 0.00 | 0.00 | 30.70 | 0.00 |
| G019005 | 0.00 | 0.00 | 1.65 | 0.35 |
| G019006 | 0.00 | 0.00 | 4.75 | 0.49 |
| G019007 | 0.00 | 0.00 | 5.35 | 0.78 |
| G019008 | 0.00 | 0.00 | 55.20 | 3.54 |
| G019009 | 0.00 | 0.00 | 19.55 | 2.19 |
| G019010 | 0.05 | 0.07 | 5.40 | 0.14 |
| G019011 | 0.00 | 0.00 | 4.40 | 0.85 |
| G019012 | 0.05 | 0.07 | 22.90 | 2.55 |
| G019013 | 0.00 | 0.00 | 30.60 | 2.40 |
| G019014 | 0.05 | 0.07 | 14.65 | 0.49 |
| G019015 | 0.00 | 0.00 | 44.70 | 1.70 |
| G019016 | 0.00 | 0.00 | 13.95 | 0.35 |
| G019017 | 0.00 | 0.00 | 2.35 | 0.35 |
| G019018 | 0.00 | 0.00 | 19.90 | 0.00 |
| G019019 | 0.00 | 0.00 | 3.20 | 0.14 |
| G021205 | 0.00 | 0.00 | 0.00 | 0.00 |
| G021206 | 0.00 | 0.00 | 4.10 | 0.28 |
| G021207 | 0.00 | 0.00 | 2.80 | 0.28 |
| G021208 | 84.75 | 2.05 | 58.50 | 0.28 |
| G021209 | 97.96 | 0.16 | 83.35 | 1.77 |
| G021210 | 71.45 | 2.90 | 75.20 | 1.70 |
| G021211 | 0.10 | 0.00 | 67.80 | 1.70 |

### Example 12—T Cell Editing, CIITA Guide RNAs with Cas9 and BC22

### 12.1 T Cell Preparation

T cells were edited at the CIITA locus with UGI in trans and either BC22 or Cas9 to assess the impact on editing type on MHC class II antigens.

T cells were prepared from a leukopak using the EasySep Human T cell Isolation Kit (Stem Cell Technology, Cat. 17951) following the manufacturers protocol. T cells were cryopreserved in Cryostor CS10 freezing media (Cat. 07930) for future use. Upon thaw, T cells were plated at a density of 1.0×10\hat{ }6 cells/mL in T cell R10 media composed of RPMI 1640 (Corning, Cat. 10-040-CV) containing 10% (v/v) of fetal bovine serum, 2 mM Glutamax (Gibco, Cat. 35050-061), 22 μM of 2-Mercaptoethanol, 100 uM non-essential amino acids (Corning, Cat. 25-025-Cl), 1 mM sodium pyruvate, 10 mM HEPES buffer, 1% of Penicillin-Streptomycin, plus 100 U/mL of recombinant human interleukin-2 (Peprotech, Cat. 200-02). T cells were activated with Dynabeads® Human T-Activator CD3/CD28 (Gibco, Cat. 11141D). Cells were expanded in T cell media for 72 hours prior to mRNA transfection.

12.2 T Cell Editing with RNA Electroporation

Solutions containing mRNA encoding Cas9 protein (SEQ ID NO: 8), BC22 (SEQ ID NO: 20) or UGI (SEQ ID NO 26) were prepared in sterile water. 50 μM CIITA targeting sgRNAs were removed from their storage plates and denatured for 2 minutes at 95° C. before cooling on ice. Seventy-two hours post activation, T cells were harvested, centrifuged, and resuspended at a concentration of 12.5×10\hat{ }6 T cells/mL in P3 electroporation buffer (Lonza). For each well to be electroporated, 1×10\hat{ }5 T cells were mixed with 200 ng of editor mRNA, 200 ng of UGI mRNA and 20 pmols of sgRNA as described in Table 33 in a final volume of 20 uL of P3 electroporation buffer. This mix was transferred in duplicate to a 96-well Nucleofector™ plate and electroporated using the manufacturer's pulse code. Electroporated T cells were rested in 180 ul of R10 media plus 100 U/mL of recombinant human interleukin-2 before being transferred to a new flat-bottom 96-well plate. The resulting plate was incubated at 37° C. for 4 days. On day 10 post-editing cells were collected for flow cytometry analysis and NGS sequencing.

### 12.3 Flow Cytometry and NGS Sequencing

On day 10 post-editing, T cells were phenotyped by flow cytometry to determine MHC class II protein expression as described in Example 6 using antibodies targeting HLA-DR, DQ, DP-PE (BioLegend® Cat. No. 361704) and Isotype Control-PE (BioLegend® Cat. No. 400234). DNA samples were subjected to PCR and subsequent NGS analysis, as described in Example 1. Table 33 shows CIITA gene editing and MHC class II negative results for cells edited with BC22. Table 34 shows CIITA gene editing and MHC class II negative results for cells edited with Cas9.

TABLE 33
Percent editing and percent of MHC-II negative cells
following CIITA editing with BC22

| | | | | | | | | | | % MHC Class II | | |
|---|---|---|---|---|---|---|---|---|---|---|---|---|
| | % C to T | | | % A to G | | | % Indel | | | negative | | |
| Guide | Mean | SD | n | Mean | SD | n | Mean | SD | n | Mean | SD | n |
| G016030 | 40.2 | 14.2 | 2 | 3.5 | 1.1 | 2 | 2.4 | 1.1 | 2 | 39.7 | 5.6 | 2 |
| G016031 | 58.8 | 0.0 | 1 | 3.4 | 3.2 | 2 | 0.6 | 0.8 | 2 | 41.5 | 3.0 | 2 |
| G016032 | 1.8 | 0.6 | 2 | 18.2 | 1.3 | 2 | 75.2 | 0.6 | 2 | 45.9 | 4.2 | 2 |
| G016033 | 1.6 | 0.1 | 2 | 30.7 | 2.2 | 2 | 18.0 | 5.2 | 2 | 38.5 | 1.5 | 2 |
| G016034 | 52.8 | 14.8 | 2 | 1.5 | 0.5 | 2 | 2.0 | 0.3 | 2 | 41.8 | 2.2 | 2 |
| G016035 | 50.3 | 14.5 | 2 | 1.6 | 0.6 | 2 | 1.4 | 0.4 | 2 | 40.8 | 2.5 | 2 |
| G016036 | 14.2 | 4.9 | 2 | 2.2 | 0.3 | 2 | 2.4 | 0.3 | 2 | 40.1 | 2.4 | 2 |
| G016037 | 10.1 | 4.5 | 2 | 1.3 | 0.4 | 2 | 0.3 | 0.1 | 2 | 38.2 | 0.8 | 2 |
| G016038 | 71.3 | 6.5 | 2 | 3.2 | 0.1 | 2 | 3.0 | 0.6 | 2 | 45.6 | 2.6 | 2 |
| G016039 | 66.0 | 5.9 | 2 | 5.0 | 0.1 | 2 | 10.5 | 2.9 | 2 | 38.6 | 0.5 | 2 |
| G016040 | No data | | | 0.0 | 0.0 | 1 | 0.0 | 0.0 | 1 | 38.4 | 0.7 | 2 |
| G016041 | No data | | | 3.1 | 4.3 | 2 | 3.3 | 1.2 | 2 | 40.4 | 2.1 | 2 |
| G016042 | 21.5 | 7.9 | 2 | 3.2 | 0.1 | 2 | 1.6 | 0.9 | 2 | 44.4 | 3.7 | 2 |
| G016043 | 44.7 | 11.5 | 2 | 2.3 | 0.1 | 2 | 3.2 | 0.5 | 2 | 40.5 | 1.6 | 2 |
| G016044 | 93.4 | 2.3 | 2 | 1.8 | 0.4 | 2 | 4.9 | 0.7 | 2 | 39.9 | 0.9 | 2 |
| G016045 | 7.1 | 3.0 | 2 | 1.2 | 0.1 | 2 | 2.2 | 0.2 | 2 | 39.8 | 0.7 | 2 |
| G016046 | 63.7 | 11.5 | 2 | 2.9 | 0.1 | 2 | 3.3 | 0.1 | 2 | 46.4 | 1.4 | 2 |
| G016047 | 72.7 | 3.0 | 2 | 2.9 | 0.2 | 2 | 4.6 | 0.9 | 2 | 45.4 | 2.0 | 2 |
| G016048 | 6.2 | 2.4 | 2 | 2.4 | 0.1 | 2 | 0.2 | 0.1 | 2 | 38.4 | 0.5 | 2 |
| G016049 | 66.8 | 9.0 | 2 | 5.4 | 0.1 | 2 | 2.8 | 0.1 | 2 | 42.4 | 2.5 | 2 |
| G016050 | 45.4 | 9.0 | 2 | 2.3 | 0.3 | 2 | 3.0 | 0.6 | 2 | 39.1 | 2.8 | 2 |
| G016051 | 86.2 | 5.9 | 2 | 3.7 | 0.1 | 2 | 1.3 | 0.3 | 2 | 39.5 | 0.6 | 2 |
| G016052 | 53.7 | 13.7 | 2 | 4.2 | 0.9 | 2 | 7.4 | 0.4 | 2 | 42.1 | 1.8 | 2 |
| G016053 | 38.6 | 18.7 | 2 | 1.3 | 0.3 | 2 | 3.1 | 0.8 | 2 | 40.4 | 1.7 | 2 |
| G016054 | 42.0 | 10.7 | 2 | 1.9 | 0.1 | 2 | 2.1 | 0.4 | 2 | 42.1 | 4.2 | 2 |
| G016055 | 36.6 | 13.1 | 2 | 3.5 | 0.9 | 2 | 8.2 | 2.3 | 2 | 41.3 | 1.5 | 2 |
| G016056 | 78.0 | 9.1 | 2 | 3.4 | 0.1 | 2 | 2.0 | 0.1 | 2 | 39.8 | 3.4 | 2 |
| G016057 | 73.3 | 9.1 | 2 | 3.4 | 0.6 | 2 | 5.3 | 0.0 | 2 | 39.7 | 1.3 | 2 |
| G016058 | 75.0 | 9.1 | 2 | 1.7 | 0.1 | 2 | 4.2 | 0.0 | 2 | 46.0 | 2.3 | 2 |
| G016059 | 66.5 | 12.0 | 2 | 4.3 | 0.2 | 2 | 4.7 | 0.7 | 2 | 41.0 | 0.8 | 2 |
| G016060 | 55.6 | 5.2 | 2 | 3.5 | 0.4 | 2 | 10.7 | 1.0 | 2 | 44.2 | 1.6 | 2 |
| G016061 | 65.5 | 9.3 | 2 | 2.5 | 0.4 | 2 | 1.2 | 0.0 | 2 | 38.6 | 2.0 | 2 |
| G016062 | 65.8 | 9.1 | 2 | 3.0 | 0.2 | 2 | 4.3 | 0.3 | 2 | 39.8 | 0.1 | 2 |
| G016063 | 10.2 | 4.2 | 2 | 0.9 | 0.2 | 2 | 0.3 | 0.1 | 2 | 39.9 | 2.5 | 2 |
| G016064 | 66.8 | 12.2 | 2 | 3.5 | 0.0 | 2 | 4.4 | 1.1 | 2 | 59.2 | 2.2 | 2 |
| G016065 | 13.5 | 5.5 | 2 | 0.6 | 0.2 | 2 | 1.1 | 0.4 | 2 | 40.4 | 2.5 | 2 |
| G016066 | 0.1 | 0.0 | 2 | 0.7 | 0.1 | 2 | 0.3 | 0.0 | 2 | 35.4 | 1.5 | 2 |
| G016067 | 82.7 | 6.4 | 2 | 2.6 | 0.6 | 2 | 4.6 | 0.2 | 2 | 59.8 | 1.2 | 2 |
| G016068 | 60.1 | 8.9 | 2 | 2.5 | 0.8 | 2 | 1.1 | 0.1 | 2 | 54.0 | 0.7 | 2 |
| G016069 | 55.8 | 11.6 | 2 | 2.6 | 0.6 | 2 | 2.6 | 1.1 | 2 | 34.3 | 4.0 | 2 |
| G016070 | 76.5 | 9.6 | 2 | 3.5 | 1.0 | 2 | 4.4 | 0.3 | 2 | 53.3 | 1.7 | 2 |
| G016071 | 54.3 | 6.8 | 2 | 4.1 | 0.5 | 2 | 1.4 | 0.6 | 2 | 45.8 | 0.1 | 2 |
| G016072 | 82.0 | 0.0 | 1 | 4.1 | 0.0 | 1 | 5.1 | 0.0 | 1 | 25.1 | 1.1 | 2 |
| G016073 | 63.4 | 11.1 | 2 | 3.5 | 0.5 | 2 | 0.9 | 0.0 | 2 | 38.7 | 1.3 | 2 |
| G016074 | 62.7 | 13.0 | 2 | 3.9 | 0.2 | 2 | 4.2 | 0.7 | 2 | 53.3 | 2.0 | 2 |
| G016075 | 41.2 | 17.2 | 2 | 1.0 | 0.4 | 2 | 8.0 | 1.7 | 2 | 48.1 | 0.7 | 2 |
| G016076 | 42.8 | 14.3 | 2 | 0.9 | 0.1 | 2 | 10.2 | 2.8 | 2 | 55.6 | 2.9 | 2 |
| G016077 | 65.1 | 10.6 | 2 | 5.4 | 0.2 | 2 | 2.4 | 0.1 | 2 | 46.0 | 0.2 | 2 |
| G016078 | 44.1 | 15.1 | 2 | 2.0 | 0.6 | 2 | 6.6 | 2.0 | 2 | 41.6 | 0.1 | 2 |
| G016079 | 79.8 | 4.9 | 2 | 5.6 | 0.5 | 2 | 4.6 | 0.9 | 2 | 53.4 | 2.8 | 2 |
| G016080 | 39.0 | 12.2 | 2 | 4.3 | 0.6 | 2 | 13.1 | 3.1 | 2 | 49.5 | 3.1 | 2 |
| G016081 | 9.6 | 4.9 | 2 | 0.5 | 0.3 | 2 | 2.5 | 0.8 | 2 | 39.0 | 0.3 | 2 |
| G016082 | 20.3 | 8.6 | 2 | 2.0 | 0.3 | 2 | 7.0 | 3.3 | 2 | 40.1 | 4.1 | 2 |
| G016083 | 74.5 | 9.8 | 2 | 3.6 | 0.1 | 2 | 8.0 | 0.6 | 2 | 48.6 | 2.3 | 2 |
| G016084 | 46.0 | 8.5 | 2 | 3.9 | 0.0 | 2 | 23.5 | 3.2 | 2 | 54.5 | 0.7 | 2 |
| G016085 | 35.6 | 6.7 | 2 | 1.4 | 0.1 | 2 | 1.6 | 0.1 | 2 | 41.9 | 3.0 | 2 |
| G016086 | 75.0 | 10.3 | 2 | 3.9 | 0.4 | 2 | 3.5 | 0.2 | 2 | 63.1 | 1.3 | 2 |
| G016087 | 45.3 | 9.9 | 2 | 1.2 | 0.1 | 2 | 3.4 | 0.5 | 2 | 40.3 | 3.7 | 2 |
| G016088 | 67.8 | 10.5 | 2 | 5.6 | 1.4 | 2 | 6.9 | 1.4 | 2 | 44.1 | 3.3 | 2 |
| G016089 | 64.4 | 10.5 | 2 | 4.5 | 0.1 | 2 | 1.5 | 0.3 | 2 | 38.3 | 1.8 | 2 |
| G016090 | 67.1 | 7.1 | 2 | 1.7 | 0.1 | 2 | 16.9 | 2.2 | 2 | 61.3 | 2.3 | 2 |
| G016091 | 47.4 | 12.0 | 2 | 2.1 | 0.9 | 2 | 2.9 | 2.2 | 2 | 54.0 | 3.9 | 2 |
| G016092 | 71.4 | 11.5 | 2 | 3.3 | 0.1 | 2 | 6.5 | 0.5 | 2 | 54.9 | 0.9 | 2 |
| G016093 | 76.6 | 8.3 | 2 | 3.3 | 0.2 | 2 | 3.7 | 0.1 | 2 | 52.2 | 2.5 | 2 |
| G016094 | 75.5 | 7.5 | 2 | 3.6 | 0.4 | 2 | 1.7 | 0.2 | 2 | 44.0 | 2.5 | 2 |
| G016095 | 76.1 | 7.2 | 2 | 7.6 | 0.4 | 2 | 2.8 | 0.4 | 2 | 44.1 | 0.4 | 2 |
| G016096 | 77.6 | 8.5 | 2 | 2.1 | 0.2 | 2 | 4.6 | 0.4 | 2 | 41.2 | 3.0 | 2 |
| G016097 | 44.7 | 15.0 | 2 | 2.2 | 0.1 | 2 | 0.7 | 0.1 | 2 | 38.6 | 3.2 | 2 |
| G016098 | 28.9 | 8.8 | 2 | 1.6 | 0.4 | 2 | 1.6 | 0.2 | 2 | 40.1 | 4.7 | 2 |
| G016099 | 68.8 | 11.9 | 2 | 2.2 | 0.1 | 2 | 3.9 | 0.7 | 2 | 44.8 | 1.0 | 2 |
| G016100 | 85.4 | 6.9 | 2 | 2.4 | 0.6 | 2 | 3.3 | 0.4 | 2 | 43.5 | 1.9 | 2 |
| G016101 | 4.8 | 1.1 | 2 | 0.8 | 0.1 | 2 | 0.2 | 0.0 | 2 | 38.0 | 3.3 | 2 |
| G016102 | 57.5 | 14.4 | 2 | 1.9 | 0.1 | 2 | 2.3 | 0.4 | 2 | 42.6 | 3.3 | 2 |
| G016103 | 69.4 | 12.8 | 2 | 2.4 | 0.0 | 2 | 5.6 | 0.5 | 2 | 39.1 | 2.7 | 2 |
| G016104 | 66.5 | 12.2 | 2 | 1.6 | 0.7 | 2 | 11.1 | 0.4 | 2 | 49.0 | 3.3 | 2 |
| G016105 | 58.4 | 14.3 | 2 | 4.4 | 0.6 | 2 | 8.3 | 0.7 | 2 | 38.4 | 3.0 | 2 |
| G016106 | 74.8 | 5.7 | 2 | 3.3 | 0.3 | 2 | 7.3 | 0.3 | 2 | 51.8 | 0.1 | 2 |
| G016107 | 45.2 | 12.2 | 2 | 5.8 | 1.1 | 2 | 5.4 | 0.8 | 2 | 39.6 | 1.0 | 2 |
| G016108 | 15.3 | 3.5 | 2 | 1.2 | 0.1 | 2 | 1.2 | 0.4 | 2 | 41.5 | 0.1 | 2 |
| G016109 | 77.5 | 3.7 | 2 | 4.5 | 0.4 | 2 | 3.4 | 0.6 | 2 | 48.3 | 2.5 | 2 |
| G016110 | 43.1 | 15.3 | 2 | 1.7 | 0.2 | 2 | 6.9 | 1.6 | 2 | 45.4 | 0.6 | 2 |
| G016111 | 89.2 | 1.3 | 2 | 4.2 | 0.1 | 2 | 5.6 | 0.8 | 2 | 40.6 | 4.4 | 2 |
| G016112 | 68.8 | 13.2 | 2 | 3.8 | 0.4 | 2 | 1.4 | 0.1 | 2 | 43.4 | 2.7 | 2 |
| G016113 | 65.2 | 9.8 | 2 | 3.6 | 0.2 | 2 | 6.9 | 1.1 | 2 | 58.3 | 2.5 | 2 |
| G016114 | 75.5 | 11.5 | 2 | 2.7 | 0.1 | 2 | 5.1 | 0.6 | 2 | 38.9 | 4.0 | 2 |
| G016115 | 72.1 | 8.6 | 2 | 1.2 | 0.1 | 2 | 7.9 | 0.3 | 2 | 60.2 | 0.9 | 2 |
| G016116 | 36.3 | 7.5 | 2 | 2.3 | 0.8 | 2 | 3.0 | 0.4 | 2 | 41.1 | 0.4 | 2 |
| G016117 | 75.4 | 7.7 | 2 | 3.5 | 0.4 | 2 | 5.9 | 0.4 | 2 | 37.1 | 2.8 | 2 |

TABLE 34
Percent editing and percent of MHC-II negative cells
following CIITA editing with Cas9

| | | | | | | | | | | % MHC Class II | | |
|---|---|---|---|---|---|---|---|---|---|---|---|---|
| | % C to T | | | % A to G | | | % Indel | | | negative | | |
| Guide | Mean | SD | n | Mean | SD | N | Mean | SD | n | Mean | SD | n |
| G016030 | 0.0 | 0.0 | 2 | 0.4 | 0.0 | 2 | 30.1 | 8.3 | 2 | 42.7 | 0.4 | 2 |
| G016031 | 0.0 | 0.0 | 1 | 0.1 | 0.0 | 1 | 79.5 | 0.0 | 1 | 45.9 | 1.0 | 2 |
| G016032 | 0.1 | 0.1 | 2 | 14.8 | 2.6 | 2 | 77.7 | 4.8 | 2 | 43.6 | 0.1 | 2 |
| G016033 | 0.1 | 0.1 | 2 | 16.1 | 2.4 | 2 | 61.5 | 5.3 | 2 | 44.1 | 1.8 | 2 |
| G016034 | 0.0 | 0.0 | 2 | 0.1 | 0.0 | 2 | 49.7 | 6.2 | 2 | 46.1 | 2.7 | 2 |
| G016035 | 0.0 | 0.0 | 2 | 0.1 | 0.0 | 2 | 44.7 | 6.4 | 2 | 46.2 | 0.6 | 2 |
| G016036 | 0.0 | 0.0 | 2 | 1.8 | 0.3 | 2 | 5.6 | 0.1 | 2 | 38.3 | 1.8 | 2 |
| G016037 | 0.1 | 0.1 | 2 | 1.2 | 0.1 | 2 | 3.4 | 0.4 | 2 | 35.9 | 1.1 | 2 |
| G016038 | 0.0 | 0.0 | 2 | 0.6 | 0.1 | 2 | 88.3 | 3.5 | 2 | 65.7 | 2.9 | 2 |
| G016039 | 0.0 | 0.0 | 2 | 0.1 | 0.0 | 2 | 91.9 | 2.6 | 2 | 62.9 | 2.5 | 2 |
| G016040 | No data | | | | | | | | | 63.2 | 0.6 | 2 |
| G016041 | No data | | | | | | | | | 62.3 | 0.6 | 2 |
| G016042 | 0.0 | 0.0 | 2 | 1.5 | 0.3 | 2 | 40.6 | 10.8 | 2 | 43.9 | 0.6 | 2 |
| G016043 | 0.0 | 0.0 | 2 | 1.3 | 0.1 | 2 | 26.7 | 4.9 | 2 | 42.9 | 0.4 | 2 |
| G016044 | No data | | | 0.2 | 0.0 | 1 | 74.4 | 0.0 | 1 | 54.9 | 0.8 | 2 |
| G016045 | 0.1 | 0.1 | 2 | 0.8 | 0.0 | 2 | 13.9 | 4.3 | 2 | 40.6 | 0.8 | 2 |
| G016046 | 0.0 | 0.0 | 2 | 0.2 | 0.1 | 2 | 92.8 | 2.4 | 2 | 62.5 | 0.6 | 2 |
| G016047 | 0.1 | 0.1 | 2 | 0.2 | 0.0 | 2 | 80.0 | 2.0 | 2 | 59.8 | 0.9 | 2 |
| G016048 | 0.0 | 0.0 | 2 | 2.1 | 0.0 | 2 | 9.3 | 2.5 | 2 | 41.0 | 0.8 | 2 |
| G016049 | 0.0 | 0.0 | 2 | 0.1 | 0.0 | 2 | 85.8 | 3.0 | 2 | 56.9 | 0.3 | 2 |
| G016050 | 0.1 | 0.1 | 2 | 0.6 | 0.1 | 2 | 38.4 | 1.6 | 2 | 45.0 | 0.4 | 2 |
| G016051 | 0.0 | 0.0 | 2 | 0.4 | 0.0 | 2 | 82.6 | 3.6 | 2 | 58.8 | 1.4 | 2 |
| G016052 | 0.1 | 0.1 | 2 | 0.6 | 0.1 | 2 | 69.8 | 7.0 | 2 | 53.8 | 0.4 | 2 |
| G016053 | No data | | | | | | | | | 43.4 | 1.2 | 2 |
| G016054 | 0.0 | 0.0 | 2 | 1.1 | 0.1 | 2 | 28.1 | 7.0 | 2 | 44.9 | 3.2 | 2 |
| G016055 | 0.0 | 0.0 | 2 | 0.6 | 0.1 | 2 | 37.1 | 7.7 | 2 | 47.5 | 0.4 | 2 |
| G016056 | 0.0 | 0.0 | 2 | 0.1 | 0.0 | 2 | 89.5 | 5.3 | 2 | 63.8 | 0.6 | 2 |
| G016057 | 0.0 | 0.0 | 2 | 0.1 | 0.0 | 2 | 84.7 | 4.0 | 2 | 61.6 | 3.1 | 2 |
| G016058 | 0.0 | 0.0 | 2 | 0.2 | 0.1 | 2 | 82.3 | 5.9 | 2 | 60.4 | 2.2 | 2 |
| G016059 | 0.0 | 0.0 | 2 | 0.1 | 0.0 | 2 | 75.1 | 3.7 | 2 | 56.6 | 1.6 | 2 |
| G016060 | 0.0 | 0.0 | 2 | 0.2 | 0.0 | 2 | 84.3 | 3.8 | 2 | 61.5 | 1.8 | 2 |
| G016061 | 0.0 | 0.0 | 2 | 0.1 | 0.0 | 2 | 55.2 | 2.9 | 2 | 47.4 | 0.6 | 2 |
| G016062 | 0.0 | 0.0 | 2 | 0.5 | 0.1 | 2 | 71.1 | 5.9 | 2 | 46.4 | 0.7 | 2 |
| G016063 | 0.0 | 0.0 | 2 | 0.6 | 0.0 | 2 | 5.1 | 1.4 | 2 | 36.1 | 1.4 | 2 |
| G016064 | 0.0 | 0.0 | 2 | 1.3 | 0.1 | 2 | 42.7 | 5.7 | 2 | 49.1 | 0.7 | 2 |
| G016065 | 0.0 | 0.0 | 2 | 0.1 | 0.0 | 2 | 11.0 | 2.0 | 2 | 40.0 | 0.9 | 2 |
| G016066 | 0.0 | 0.0 | 2 | 0.6 | 0.0 | 2 | 0.4 | 0.1 | 2 | 36.8 | 0.6 | 2 |
| G016067 | 0.1 | 0.0 | 2 | 0.1 | 0.0 | 2 | 85.4 | 3.3 | 2 | 59.7 | 3.3 | 2 |
| G016068 | 0.0 | 0.0 | 2 | 0.1 | 0.0 | 2 | 59.2 | 6.9 | | 54.0 | 0.2 | 2 |
| G016069 | 0.0 | 0.0 | 2 | 0.4 | 0.0 | 2 | 39.5 | 3.7 | 2 | 40.7 | 0.0 | 2 |
| G016070 | 0.1 | 0.1 | 2 | 0.1 | 0.1 | 2 | 92.3 | 2.3 | 2 | 66.4 | 3.1 | 2 |
| G016071 | 0.0 | 0.0 | 2 | 0.2 | 0.0 | 2 | 73.1 | 2.2 | 2 | 55.6 | 3.0 | 2 |
| G016072 | 0.0 | 0.0 | 2 | 0.1 | 0.1 | 2 | 91.1 | 1.4 | 2 | 51.6 | 1.1 | 2 |
| G016073 | 0.0 | 0.0 | 2 | 1.5 | 0.1 | 2 | 38.3 | 4.2 | 2 | 43.5 | 0.8 | 2 |
| G016074 | 0.2 | 0.1 | 2 | 0.6 | 0.0 | 2 | 72.4 | 7.5 | 2 | 56.1 | 1.4 | 2 |
| G016075 | 0.1 | 0.1 | 2 | 0.3 | 0.1 | 2 | 72.9 | 10.3 | 2 | 57.8 | 4.4 | 2 |
| G016076 | 0.0 | 0.0 | 2 | 0.3 | 0.1 | 2 | 66.7 | 13.4 | 2 | 58.4 | 4.6 | 2 |
| G016077 | 0.0 | 0.0 | 2 | 0.5 | 0.1 | 2 | 80.0 | 5.2 | 2 | 62.5 | 1.5 | 2 |
| G016078 | 0.1 | 0.1 | 2 | 0.3 | 0.2 | 2 | 59.3 | 10.5 | 2 | 51.6 | 4.5 | 2 |
| G016079 | 0.0 | 0.0 | 2 | 0.7 | 0.1 | 2 | 81.9 | 4.0 | 2 | 58.4 | 1.0 | 2 |
| G016080 | 0.0 | 0.0 | 2 | 0.5 | 0.1 | 2 | 71.8 | 6.2 | 2 | 44.9 | 1.4 | 2 |
| G016081 | 0.0 | 0.0 | 2 | 0.4 | 0.0 | 2 | 8.1 | 1.3 | 2 | 39.1 | 0.7 | 2 |
| G016082 | 0.1 | 0.1 | 2 | 2.1 | 0.0 | 2 | 10.0 | 2.5 | 2 | 39.0 | 0.8 | 2 |
| G016083 | 0.1 | 0.1 | 2 | 0.2 | 0.1 | 2 | 92.2 | 1.5 | 2 | 63.6 | 0.7 | 2 |
| G016084 | 0.0 | 0.0 | 2 | 0.4 | 0.0 | 2 | 70.7 | 6.4 | 2 | 56.1 | 2.6 | 2 |
| G016085 | 0.0 | 0.0 | 2 | 0.3 | 0.1 | 2 | 17.5 | 0.7 | 2 | 42.3 | 0.4 | 2 |
| G016086 | 0.1 | 0.1 | 2 | 0.2 | 0.1 | 2 | 85.8 | 6.1 | 2 | 62.2 | 3.0 | 2 |
| G016087 | 0.0 | 0.0 | 2 | 0.2 | 0.0 | 2 | 89.5 | 2.1 | 2 | 56.1 | 0.1 | 2 |
| G016088 | 0.8 | 0.0 | 2 | 0.3 | 0.1 | 2 | 76.8 | 4.6 | 2 | 58.1 | 0.5 | 2 |
| G016089 | 0.1 | 0.1 | 2 | 0.3 | 0.1 | 2 | 73.3 | 6.4 | 2 | 54.2 | 0.0 | 2 |
| G016090 | 0.2 | 0.0 | 2 | 0.3 | 0.0 | 2 | 88.3 | 5.1 | 2 | 61.2 | 2.3 | 2 |
| G016091 | 0.0 | 0.0 | 1 | 0.7 | 0.0 | 1 | 42.0 | 0.0 | 1 | 49.5 | 3.8 | 2 |
| G016092 | 0.1 | 0.1 | 2 | 0.5 | 0.1 | 2 | 60.9 | 10.0 | 2 | 52.0 | 2.9 | 2 |
| G016093 | 0.0 | 0.0 | 2 | 0.5 | 0.1 | 2 | 68.8 | 8.1 | 2 | 50.6 | 2.5 | 2 |
| G016094 | 0.1 | 0.1 | 2 | 0.1 | 0.0 | 2 | 71.3 | 6.5 | 2 | 50.5 | 3.3 | 2 |
| G016095 | 0.0 | 0.0 | 2 | 0.6 | 0.1 | 2 | 70.5 | 5.4 | 2 | 51.6 | 5.0 | 2 |
| G016096 | 0.2 | 0.1 | 2 | 0.1 | 0.0 | 2 | 94.9 | 2.0 | 2 | 51.2 | 0.1 | 2 |
| G016097 | 0.1 | 0.1 | 2 | 0.3 | 0.0 | 2 | 39.7 | 12.4 | 2 | 50.9 | 4.5 | 2 |
| G016098 | 0.1 | 0.1 | 2 | 0.2 | 0.0 | 2 | 23.4 | 7.5 | 2 | 47.2 | 0.5 | 2 |
| G016099 | 0.1 | 0.0 | 2 | 0.2 | 0.0 | 2 | 84.7 | 5.8 | 2 | 63.2 | 2.5 | 2 |
| G016100 | 0.0 | 0.0 | 2 | 0.3 | 0.1 | 2 | 79.8 | 7.1 | 2 | 60.3 | 0.6 | 2 |
| G016101 | 0.1 | 0.1 | 2 | 0.6 | 0.1 | 2 | 2.3 | 0.8 | 2 | 38.8 | 1.5 | 2 |
| G016102 | 0.0 | 0.0 | 2 | 0.4 | 0.1 | 2 | 75.7 | 8.9 | 2 | 59.9 | 7.1 | 2 |
| G016103 | 0.2 | 0.1 | 2 | 0.6 | 0.1 | 2 | 76.8 | 4.7 | 2 | 46.9 | 3.4 | 2 |
| G016104 | 1.4 | 0.0 | 1 | 1.1 | 0.0 | 1 | 66.8 | 0.0 | 1 | 56.1 | 3.1 | 2 |
| G016105 | 0.1 | 0.1 | 2 | 0.7 | 0.3 | 2 | 90.7 | 5.1 | 2 | 58.0 | 3.0 | 2 |
| G016106 | 0.0 | 0.0 | 2 | 0.2 | 0.0 | 2 | 95.1 | 2.1 | 2 | 62.2 | 3.0 | 2 |
| G016107 | 0.1 | 0.1 | 2 | 0.2 | 0.0 | 2 | 84.9 | 2.6 | 2 | 59.5 | 1.1 | 2 |
| G016108 | 0.0 | 0.0 | 2 | 0.6 | 0.1 | 2 | 19.1 | 4.8 | 2 | 43.3 | 0.8 | 2 |
| G016109 | 0.0 | 0.0 | 2 | 0.1 | 0.0 | 2 | 86.5 | 3.3 | 2 | 62.9 | 3.5 | 2 |
| G016110 | 0.0 | 0.0 | 2 | 0.6 | 0.1 | 2 | 34.9 | 10.0 | 2 | 48.0 | 4.2 | 2 |
| G016111 | 65.6* | 3.7 | 2 | 1.4 | 0.1 | 2 | 32.9 | 3.7 | 2 | 44.5 | 1.3 | 2 |
| G016112 | 0.0 | 0.0 | 2 | 0.8 | 0.1 | 2 | 60.7 | 6.7 | 2 | 54.1 | 5.3 | 2 |
| G016113 | 1.1 | 0.4 | 2 | 0.2 | 0.0 | 2 | 84.2 | 6.7 | 2 | 60.5 | 5.2 | 2 |
| G016114 | 0.1 | 0.1 | 2 | 0.4 | 0.0 | 2 | 87.6 | 3.5 | 2 | 50.2 | 2.5 | 2 |
| G016115 | 0.0 | 0.0 | 2 | 0.3 | 0.1 | 2 | 69.3 | 7.4 | 2 | 52.8 | 4.8 | 2 |
| G016116 | 0.0 | 0.0 | 2 | 0.4 | 0.0 | 2 | 16.6 | 5.0 | 2 | 38.7 | 0.4 | 2 |
| G016117 | 0.0 | 0.0 | 2 | 0.4 | 0.1 | 2 | 85.6 | 4.9 | 2 | 49.0 | 3.0 | 2 |

*There is a naturally occurring C/T single nucleotide polymorphism for G016111 target sequence.

### Example 13—Dose Response and Multiplexed Editing

Three guides from Table 33, G016086, G016092, and G016067, were further characterized for editing efficacy with increasing amounts of guide and in combination with guides targeting TRAC (G013009, G016016, or G016017) and B32M (G015991, G015995, or G015996).

Cell preparation, activation, and electroporation were performed as described in Example 6 with the following deviations. Editing was performed using two miRNA species encoding BC22 (SEQ ID NO: 20) and UGI (SEQ ID NO:26) respectively. Editing was assessed at multiple concentrations of sgRNA, as indicated in Table 35 and Table 36. When multiple guides were used in a single reaction, each guide represented one quarter of the total guide concentration.

On day 10 post-editing, T cells were phenotyped by flow cytometry to determine MHC class II protein expression as described in Example 6. In addition, B32M detection was performed with B2M-FITC antibody (BioLegend, Cat. 316304) and CD3 expression was assayed using CD3-BV605 antibody (BioLegend, Cat. 317322).

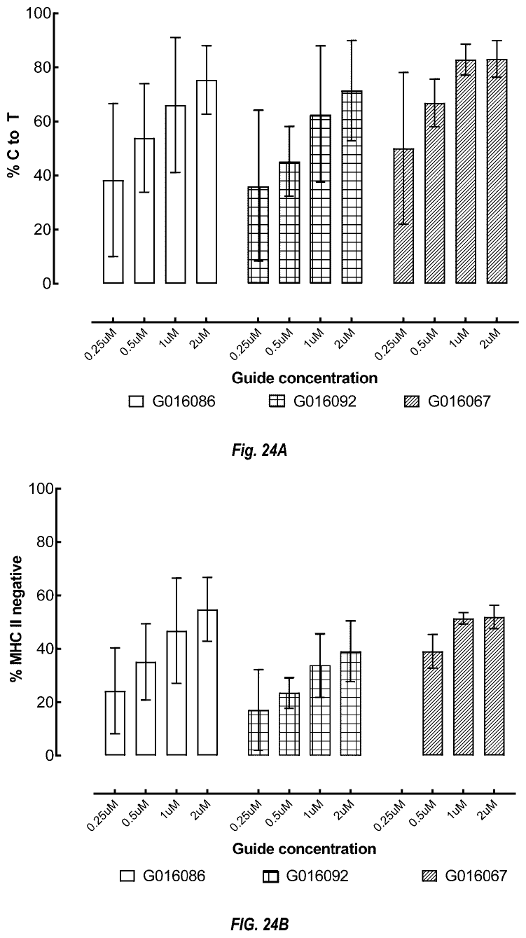

DNA samples were subjected to PCR and subsequent NGS analysis, as described in Example 1. Table 35 provides MHC Class II negative flow cytometry results and NGS editing for cells edited with BC22 and individual guides targeting CIITA, with FIG. 24A graphing the percent C-to-T conversion and FIG. 243 graphing the percent MHC class II negative. Table 36 shows MHC Class II negative results for cells edited simultaneously with CIITA, B2M, and TRAC guides.

TABLE 35
Percent MHC-II negative cells and NGS outcomes following CIITA editing (n = 2)

| Guide Concentration | | 2 uM | | 1 uM | | 0.5 uM | | 0.25 uM | |
|---|---|---|---|---|---|---|---|---|---|
| Assay | Guide | Mean | SD | Mean | SD | Mean | SD | Mean | SD |
| MHC-II | G016086 | 80.2 | 12.0 | 72.2 | 19.7 | 60.5 | 14.3 | 49.7 | 16.1 |
| neg | G016092 | 64.5 | 11.4 | 59.3 | 11.8 | 49.0 | 5.7 | 42.6 | 15.1 |
| | G016067 | 77.3 | 4.4 | 76.8 | 2.1 | 64.5 | 6.3 | | |
| C-to-T | G016086 | 75.4 | 12.7 | 66.1 | 25.0 | 53.9 | 20.1 | 38.3 | 28.3 |
| | G016092 | 71.5 | 18.5 | 62.5 | 25.5 | 45.2 | 13.0 | 36.0 | 28.1 |
| | G016067 | 83.1 | 6.8 | 82.9 | 5.7 | 66.9 | 8.8 | 50.1 | 28.1 |
| C-to-A/G | G016086 | 1.8 | 0.1 | 1.6 | 0.1 | 1.7 | 0.1 | 1.1 | 0.6 |
| | G016092 | 1.6 | 0.1 | 1.3 | 0.2 | 1.2 | 0.0 | 1.2 | 0.5 |
| | G016067 | 1.0 | 0.3 | 1.2 | 0.1 | 1.5 | 0.5 | 0.9 | 0.5 |
| Indel | G016086 | 2.5 | 1.4 | 1.4 | 0.5 | 1.5 | 0.3 | 1.2 | 0.5 |
| | G016092 | 2.9 | 0.3 | 3.0 | 0.6 | 3.1 | 0.5 | 2.5 | 1.7 |
| | G016067 | 3.3 | 0.1 | 2.4 | 0.0 | 3.4 | 1.4 | 2.1 | 1.2 |

TABLE 36
Percent antigen negative cells following CIITA, TRAC, and
B2M editing

| | Concentration | | | | | | |
|---|---|---|---|---|---|---|---|
| | per guide: | 0.5 uM | | 0.25 uM | | 0.125 uM | |
| Assay | Guide | Mean | SD | Mean | SD | Mean | SD |
| Triple | G015995 G016086 | 62.1 | 9.3 | 51.9 | 9.4 | 26.1 | 13.6 |
| Neg | G016017 | | | | | | |
| | G015991 G016092 | 43.8 | 15.6 | 20.2 | 7.4 | 8.2 | 7.6 |
| | G016016 | | | | | | |
| | G015996 G016067 | 35.3 | 17.7 | 15.4 | 12.3 | 6.8 | 7.3 |
| | G013009 | | | | | | |
| MHC | G015995 G016086 | 67.0 | 8.0 | 57.6 | 7.8 | 38.6 | 5.4 |
| Class II | G016017 | | | | | | |
| Neg | G015991 G016092 | 57.2 | 8.2 | 45.2 | 3.2 | 36.8 | 5.3 |
| | G016016 | | | | | | |
| | G015996 G016067 | 53.1 | 7.9 | 69.1 | 43.1 | 41.3 | 7.1 |
| | G013009 | | | | | | |
| CD3 | G015995 G016086 | 92.5 | 2.3 | 90.5 | 3.2 | 77.3 | 15.1 |
| Neg | G016017 | | | | | | |
| | G015991 G016092 | 88.1 | 6.2 | 87.6 | 3.2 | 74.2 | 14.0 |
| | G016016 | | | | | | |
| | G015996 G016067 | 92.8 | 2.0 | 89.5 | 4.8 | 79.3 | 14.4 |
| | G013009 | | | | | | |
| B2M | G015995 G016086 | 94.9 | 2.5 | 90.0 | 6.4 | 63.2 | 28.0 |
| Neg | G016017 | | | | | | |
| | G015991 G016092 | 73.4 | 19.2 | 29.1 | 13.1 | 14.3 | 15.2 |
| | G016016 | | | | | | |
| | G015996 G016067 | 60.8 | 25.7 | 29.1 | 21.4 | 14.3 | 15.0 |
| | G013009 | | | | | | |

### Example 14. Screening of TRAC Guide RNAs

TRAC gRNAs were screened for efficacy in T cells by assessing knockdown of CD3 surface expression using both Cas9 with UGI in trans and BC22 with UGI in trans. The percentage of T cells negative for CD3 protein and the percentage of editing at the TRAC locus was assayed following TRAC editing by electroporation with mRNA and gRNA.

### Example 14.1. T Cell Preparation

T cells were prepared from a leukopak using the EasySep Human T cell Isolation Kit (Stem Cell Technology, Cat. 17951) following the manufacturers protocol. T cells were cryopreserved in Cryostor CS10 freezing media (Cat. 07930) for future use. Upon thaw, T cells were plated at a density of 1.0×10\hat{ }6 cells/mL in T cell R10 media composed of RPMI 1640 (Corning, Cat. 10-040-CV) containing 10% (v/v) of fetal bovine serum, 2 mM Glutamax (Gibco, Cat. 35050-061), 22 μM of 2-Mercaptoethanol, 100 uM non-essential amino acids (Corning, Cat. 25-025-Cl), 1 mM sodium pyruvate, 10 mM HEPES buffer, 1% of Penicillin-Streptomycin, plus 100 U/mL of recombinant human interleukin-2 (Peprotech, Cat. 200-02). T cells were activated with Dynabeads® Human T-Activator CD3/CD28 (Gibco, Cat. 11141D). Cells were expanded in T cell media for 72 hours prior to mRNA transfection.

### Example 14.2. T Cell Editing with RNA Electroporation

Solutions containing mRNA encoding Cas9 protein (SEQ ID NO: 8), BC22 (SEQ ID NO: 20) or UGI (SEQ ID NO 26) were prepared in sterile water. 50 μM TRAC targeting sgRNAs were removed from their storage plates and denatured for 2 minutes at 95° C. before cooling on ice. Seventy-two hours post activation, T cells were harvested, centrifuged, and resuspended at a concentration of 12.5×10\hat{ }6 T cells/mL in P3 electroporation buffer (Lonza). For each well to be electroporated, 1×10\hat{ }5 T cells were mixed with 200 ng of editor mRNA, 200 ng of UGI mRNA and 20 pmols of sgRNA as described in Table 37 in a final volume of 20 uL of P3 electroporation buffer. This mix was transferred in triplicate to a 96-well Nucleofector™ plate and electroporated using the manufacturer's pulse code. Electroporated T cells were rested in 180 ul of R10 media plus 100 U/mL of recombinant human interleukin-2 before being transferred to a new flat-bottom 96-well plate. The resulting plate was incubated at 37° C. for 6 days. On day 9 post-editing cells were collected for flow cytometry analysis and NGS sequencing.

### Example 14.3. Flow Cytometry and NGS Sequencing

On day 9 post-editing, T cells were phenotyped by flow cytometry to determine CD3 protein expression as described in Example 6 using antibodies targeting CD3 (BioLegend® Cat. No. 317322) and Isotype Control-PE (BioLegend® Cat. No. 400269). DNA samples were subjected to PCR and subsequent NGS analysis, as described in Example 1. The mean percentage of each editing type at the TRAC locus and the mean number of CD3 negative cells after editing with BC22 and UGI are showing in Table 37; results after editing with Cas9 and UGI are shown in Table 38. C-to-T editing purity is calculated as the percentage of edited reads containing only C-to-T conversions.

TABLE 37
Percent editing and percent of CD3 negative cells following TRAC editing with
BC22 and UGI

| | | | | | | | % C to T | | | | | |
|---|---|---|---|---|---|---|---|---|---|---|---|---|
| | % C to T | | % A to G | | % Indel | | purity | | | % CD3 negative | | |
| Guide | Mean | SD | Mean | SD | Mean | SD | Mean | SD | n | Mean | SD | n |
| G012086 | 95.21 | 0.42 | 2.46 | 0.16 | 1.24 | 0.04 | 96.26 | 0.12 | 3 | 1.22 | 0.55 | 3 |
| G013004 | 74.7 | 14.43 | 1.6 | 0.32 | 3.25 | 1.05 | 93.97 | 0.52 | 3 | 17.17 | 7.69 | 3 |
| G013005 | 89.47 | 0.6 | 3.24 | 0.11 | 4.04 | 0.49 | 92.48 | 0.53 | 3 | 8.52 | 1.59 | 3 |
| G013006 | 90.56 | 3.87 | 2.82 | 0.23 | 1.74 | 0.15 | 95.21 | 0.16 | 3 | 2.77 | 0.34 | 3 |
| G013007 | 90.96 | 3.68 | 3.05 | 0.37 | 1.51 | 0.35 | 95.21 | 0.37 | 3 | 3.07 | 1.2 | 3 |
| G013008 | 67.48 | 24.46 | 2.15 | 0.67 | 1.07 | 0.51 | 95.45 | 0.64 | 3 | 1.2 | 0.38 | 3 |
| G013009 | 90.05 | 4.17 | 2.29 | 0.27 | 2.09 | 0.17 | 95.36 | 0.36 | 3 | 87.87 | 5.02 | 3 |
| G014831 | 92.48 | 1.55 | 2.26 | 0.22 | 2.93 | 0.25 | 94.68 | 0.54 | 3 | 50.5 | 3.25 | 3 |
| G015997 | 86.72 | 9.45 | 1.34 | 0.09 | 1.18 | 0.21 | 97.14 | 0.50 | 3 | 2.45 | 0.75 | 3 |
| G015998 | 79.5 | 15.18 | 2.12 | 0.63 | 0.98 | 0.12 | 96.25 | 0.13 | 3 | 2.8 | 1.35 | 3 |
| G015999 | 87.16 | 1.81 | 3.57 | 0.03 | 5.8 | 0.77 | 90.30 | 0.57 | 3 | 14.16 | 6.97 | 3 |
| G016000 | 74.27 | 15.17 | 2.79 | 0.16 | 1.6 | 0.16 | 94.24 | 1.47 | 3 | 6.64 | 0.97 | 3 |
| G016001 | 96.49 | 1.69 | 0.88 | 0.14 | 0.42 | 0.06 | 98.67 | 0.22 | 3 | 15.93 | 3.4 | 3 |
| G016002 | 96.11 | 0.65 | 1.18 | 0.1 | 0.87 | 0.07 | 97.90 | 0.09 | 3 | 4.18 | 2.8 | 3 |
| G016003 | 84.79 | 6.22 | 1.04 | 0.06 | 7.1 | 1.92 | 91.24 | 1.99 | 3 | 2.17 | 1.04 | 3 |
| G016004 | 91.78 | 3.75 | 2.16 | 0.18 | 0.95 | 0.23 | 96.72 | 0.36 | 3 | 1.53 | 0.28 | 3 |
| G016005 | 91.02 | 3.02 | 2.16 | 0.08 | 3.06 | 0.13 | 94.58 | 0.11 | 3 | 15.37 | 2.72 | 3 |
| G016006 | 92.18 | 1.4 | 1.91 | 0.09 | 2.97 | 0.05 | 94.98 | 0.09 | 3 | 22.03 | 0.9 | 3 |
| G016007 | 96.71 | 0.32 | 1.14 | 0.12 | 1.05 | 0.2 | 97.78 | 0.22 | 3 | 5.78 | 0.2 | 3 |
| G016008 | 95.81 | 0.94 | 1.29 | 0.07 | 1.33 | 0.2 | 97.34 | 0.16 | 3 | 36.67 | 0.45 | 3 |
| G016009 | 38.69 | 13.44 | 2.08 | 0.22 | 0.4 | 0.17 | 93.73 | 1.11 | 3 | 0.79 | 0.2 | 3 |
| G016010 | 91.39 | 0.55 | 2.22 | 0.06 | 5.02 | 0.43 | 92.67 | 0.47 | 3 | 9.21 | 0.44 | 3 |
| G016011 | 94.67 | 0.78 | 2.26 | 0.11 | 1.04 | 0.1 | 96.63 | 0.16 | 3 | 0.32 | 0.06 | 3 |
| G016012 | 87.92 | 1.23 | 2 | 0.17 | 7.99 | 1.51 | 89.80 | 1.36 | 3 | 10.11 | 0.54 | 3 |
| G016013 | 91.68 | 3.86 | 1.77 | 0.06 | 2.45 | 0.28 | 95.59 | 0.43 | 3 | 59.4 | 4.51 | 3 |
| G016014 | 90.7 | 6.53 | 1.6 | 0.02 | 1.44 | 0.26 | 96.75 | 0.30 | 3 | 6.21 | 2.09 | 3 |
| G016015 | 93.07 | 2.04 | 2.84 | 0.16 | 1.2 | 0.23 | 95.83 | 0.41 | 3 | 0.88 | 0.67 | 3 |
| G016016 | 94.2 | 0.99 | 2.11 | 0.11 | 0.96 | 0.18 | 96.84 | 0.28 | 3 | 90.8 | 1.59 | 3 |
| G016017 | 95.51 | 0.63 | 1.86 | 0.1 | 1.04 | 0.17 | 97.05 | 0.25 | 3 | 91.1 | 1.35 | 3 |
| G016018 | 94.5 | 1.03 | 1.48 | 0.16 | 1.61 | 0.24 | 96.83 | 0.34 | 3 | 6.02 | 1.95 | 3 |
| G016019 | 71.54 | 5.93 | 4.69 | 0.23 | 3.03 | 0.53 | 90.27 | 0.14 | 2 | 78.13 | 9.8 | 3 |
| G016020 | 29.14 | 9.23 | 1.64 | 0.35 | 0.44 | 0.18 | 93.23 | 0.87 | 3 | 1.56 | 0.8 | 3 |
| G016021 | 32.46 | 10.32 | 2.07 | 0.54 | 1.42 | 0.21 | 90.09 | 0.99 | 3 | 0.65 | 0.13 | 3 |
| G016022 | 89.76 | 4.52 | 1.98 | 0.03 | 2.74 | 0.16 | 95.00 | 0.11 | 3 | 15.97 | 6.07 | 3 |
| G016023 | 47.93 | 14.11 | 2.17 | 0.64 | 1.72 | 0.39 | 92.48 | 0.21 | 2 | 5.12 | 2.45 | 3 |
| G016024 | 91.23 | 3.82 | 1.77 | 0.27 | 2.48 | 0.28 | 95.55 | 0.37 | 3 | 3.56 | 2.12 | 3 |
| G016025 | 59.93 | 18.15 | 1.18 | 0.3 | 0.71 | 0.06 | 96.86 | 0.40 | 3 | 1.86 | 0.37 | 3 |
| G016026 | 93.52 | 3.27 | 1.45 | 0.29 | 0.8 | 0.09 | 97.65 | 0.29 | 3 | 82.27 | 3.13 | 3 |
| G016027 | 92.7 | 1.71 | 1.67 | 0.04 | 3.09 | 0.19 | 95.12 | 0.16 | 3 | 5.05 | 4.02 | 3 |
| G016028 | 84.88 | 9.36 | 1.29 | 0.11 | 0.88 | 0.05 | 97.49 | 0.29 | 3 | 2.01 | 1.07 | 3 |
| G016029 | 51.08 | 14.8 | 1.57 | 0.37 | 0.8 | 0.08 | 95.48 | 0.50 | 3 | 1.29 | 1.14 | 3 |

TABLE 38
Percent editing and percent of CD3 negative cells following TRAC editing with
Cas9

| | % C to T | | | % A to G | | | % Indel | | | % CD3 negative | | |
|---|---|---|---|---|---|---|---|---|---|---|---|---|
| Guide | Mean | SD | n | Mean | SD | n | Mean | SD | n | Mean | SD | n |
| G012086 | 0.01 | 0.01 | 3 | 0.10 | 0.02 | 3 | 98.18 | 0.28 | 3 | 94.87 | 0.25 | 3 |
| G013004 | 0.05 | 0.01 | 3 | 0.17 | 0.08 | 3 | 64.90 | 20.08 | 3 | 61.17 | 19.91 | 3 |
| G013005 | 0.03 | 0.00 | 3 | 0.05 | 0.01 | 3 | 94.14 | 1.85 | 3 | 83.43 | 2.05 | 3 |
| G013006 | 0.04 | 0.03 | 3 | 0.11 | 0.07 | 3 | 96.29 | 2.22 | 3 | 92.90 | 1.32 | 3 |
| G013007 | 0.05 | 0.01 | 3 | 0.11 | 0.02 | 3 | 96.13 | 1.88 | 3 | 92.93 | 1.69 | 3 |
| G013008 | 0.04 | 0.01 | 3 | 0.23 | 0.11 | 3 | 83.46 | 9.24 | 3 | 79.53 | 7.89 | 3 |
| G013009 | 0.19 | 0.05 | 3 | 0.20 | 0.02 | 3 | 94.74 | 2.23 | 3 | 80.87 | 1.65 | 3 |
| G014831 | 0.02 | 0.02 | 3 | 0.07 | 0.02 | 3 | 99.13 | 0.19 | 3 | 94.87 | 0.21 | 3 |
| G015997 | 0.19 | 0.07 | 3 | 0.16 | 0.01 | 3 | 90.71 | 4.41 | 3 | 88.77 | 3.65 | 3 |
| G015998 | 0.22 | 0.06 | 3 | 0.15 | 0.04 | 3 | 93.52 | 4.46 | 3 | 80.93 | 1.07 | 3 |
| G015999 | 0.02 | 0.02 | 3 | 0.01 | 0.01 | 3 | 98.15 | 0.89 | 3 | 87.00 | 0.78 | 3 |
| G016000 | 0.05 | 0.03 | 3 | 0.08 | 0.01 | 3 | 97.63 | 0.90 | 3 | 92.87 | 0.93 | 3 |
| G016001 | 0.01 | 0.01 | 3 | 0.01 | 0.01 | 3 | 95.81 | 1.95 | 3 | 83.63 | 1.05 | 3 |
| G016002 | 0.02 | 0.02 | 3 | 0.08 | 0.07 | 3 | 97.47 | 0.51 | 3 | 32.97 | 3.19 | 3 |
| G016003 | 0.17 | 0.08 | 3 | 0.08 | 0.03 | 3 | 95.15 | 3.25 | 3 | 70.80 | 2.09 | 3 |
| G016004 | 0.06 | 0.06 | 3 | 0.13 | 0.07 | 3 | 97.56 | 0.99 | 3 | 93.83 | 0.81 | 3 |
| G016005 | 0.22 | 0.07 | 3 | 0.21 | 0.07 | 3 | 98.75 | 0.01 | 3 | 87.40 | 1.47 | 3 |
| G016006 | 0.03 | 0.03 | 3 | 0.14 | 0.06 | 3 | 97.76 | 0.66 | 3 | 94.93 | 0.47 | 3 |
| G016007 | 0.09 | 0.04 | 3 | 0.06 | 0.03 | 3 | 98.40 | 0.46 | 3 | 94.73 | 0.40 | 3 |
| G016008 | 0.00 | 0.01 | 3 | 0.01 | 0.01 | 3 | 98.92 | 0.35 | 3 | 94.67 | 0.55 | 3 |
| G016009 | 0.19 | 0.05 | 3 | 0.77 | 0.11 | 3 | 13.55 | 3.43 | 3 | 68.13 | 3.92 | 3 |
| G016010 | 0.02 | 0.00 | 3 | 0.19 | 0.04 | 3 | 98.20 | 0.40 | 3 | 93.63 | 0.21 | 3 |
| G016011 | 0.06 | 0.02 | 3 | 0.18 | 0.03 | 3 | 97.57 | 0.73 | 3 | 93.03 | 63.50 | 3 |
| G016012 | 0.04 | 0.02 | 3 | 0.12 | 0.05 | 3 | 97.37 | 0.15 | 3 | 93.87 | 0.76 | 3 |
| G016013 | 0.03 | 0.04 | 3 | 0.05 | 0.00 | 3 | 97.49 | 1.15 | 3 | 82.40 | 2.11 | 3 |
| G016014 | 0.00 | 0.00 | 3 | 0.04 | 0.06 | 3 | 98.15 | 1.00 | 3 | 92.43 | 0.90 | 3 |
| G016015 | 0.07 | 0.05 | 3 | 0.19 | 0.03 | 3 | 98.09 | 0.20 | 3 | 94.03 | 0.29 | 3 |
| G016016 | 0.01 | 0.01 | 3 | 0.05 | 0.01 | 3 | 97.03 | 0.81 | 3 | 84.17 | 0.29 | 3 |
| G016017 | 0.03 | 0.03 | 3 | 0.05 | 0.01 | 3 | 97.64 | 0.43 | 3 | 77.80 | 4.97 | 3 |
| G016018 | 0.05 | 0.03 | 3 | 0.03 | 0.01 | 3 | 98.44 | 0.32 | 3 | 61.57 | 6.82 | 3 |
| G016019 | 0.01 | 0.00 | 3 | 0.06 | 0.02 | 3 | 88.15 | 5.31 | 3 | 31.43 | 5.54 | 3 |
| G016020 | 0.03 | 0.02 | 3 | 0.10 | 0.03 | 3 | 79.18 | 7.16 | 3 | 93.30 | 1.28 | 3 |
| G016021 | 0.03 | 0.01 | 3 | 0.23 | 0.04 | 3 | 42.54 | 6.06 | 3 | 70.13 | 8.45 | 3 |
| G016022 | 0.00 | 0.00 | 3 | 0.01 | 0.01 | 3 | 98.01 | 1.02 | 3 | 94.23 | 0.72 | 3 |
| G016023 | 0.07 | 0.03 | 3 | 0.54 | 0.10 | 3 | 88.15 | 7.96 | 3 | 51.13 | 15.47 | 3 |
| G016024 | 0.02 | 0.02 | 3 | 0.24 | 0.13 | 3 | 98.29 | 0.54 | 3 | 76.57 | 2.92 | 3 |
| G016025 | 0.15 | 0.02 | 3 | 0.24 | 0.04 | 3 | 58.36 | 15.14 | 3 | 62.27 | 3.41 | 3 |
| G016026 | 0.00 | 0.01 | 3 | 0.03 | 0.01 | 3 | 95.34 | 1.69 | 3 | 65.37 | 10.10 | 3 |
| G016027 | 0.08 | 0.01 | 3 | 0.27 | 0.14 | 3 | 92.27 | 3.82 | 3 | 15.07 | 5.42 | 3 |
| G016028 | 0.07 | 0.06 | 3 | 0.16 | 0.05 | 3 | 73.05 | 9.53 | 3 | 90.10 | 0.28 | 2 |
| G016029 | 0.09 | 0.01 | 3 | 0.51 | 0.04 | 3 | 19.24 | 5.53 | 3 | 95.00 | 0.28 | 2 |

### Example 15. Screening of TRBC Guide RNAs

TRBC gRNAs were screened for efficacy in T cells by assessing knockdown of CD3 surface expression using both Cas9 with UGI in trans and BC22 with UGI in trans. The percentage of T cells negative for CD3 protein and the percentage of editing of each type at the TRBC1 locus was assayed following TRBC editing by electroporation with mRNA and gRNA.

### Example 15.1. T Cell Preparation

T cells were prepared from a leukopak using the EasySep Human T cell Isolation Kit (Stem Cell Technology, Cat. 17951) following the manufacturers protocol. T cells were cryopreserved in Cryostor CS10 freezing media (Cat. 07930) for future use. Upon thaw, T cells were plated at a density of 1.0×10\hat{ }6 cells/mL in T cell R10 media composed of RPMI 1640 (Corning, Cat. 10-040-CV) containing 10% (v/v) of fetal bovine serum, 2 mM Glutamax (Gibco, Cat. 35050-061), 22 μM of 2-Mercaptoethanol, 100 uM non-essential amino acids (Corning, Cat. 25-025-Cl), 1 mM sodium pyruvate, 10 mM HEPES buffer, 1% of Penicillin-Streptomycin, plus 100 U/mL of recombinant human interleukin-2 (Peprotech, Cat. 200-02). T cells were activated with Dynabeads® Human T-Activator CD3/CD28 (Gibco, Cat. 11141D). Cells were expanded in T cell media for 72 hours prior to mRNA transfection.

### Example 15.2. T Cell Editing with RNA Electroporation

Solutions containing mRNA encoding Cas9 protein (SEQ ID NO: 8), BC22 (SEQ ID NO: 20) or UGI (SEQ ID NO 26) were prepared in sterile water. 50 μM TRBC targeting sgRNAs were removed from their storage plates and denatured for 2 minutes at 95° C. before cooling on ice. Seventy-two hours post activation, T cells were harvested, centrifuged, and resuspended at a concentration of 12.5×10\hat{ }6 T cells/mL in P3 electroporation buffer (Lonza). For each well to be electroporated, 1×10\hat{ }5 T cells were mixed with 200 ng of editor mRNA, 200 ng of UGI mRNA and 20 pmols of sgRNA as described in Table 39 in a final volume of 20 uL of P3 electroporation buffer. This mix was transferred in triplicate to a 96-well Nucleofector™ plate and electroporated using the manufacturer's pulse code. Electroporated T cells were rested in 180 ul of R10 media plus 100 U/mL of recombinant human interleukin-2 before being transferred to a new flat-bottom 96-well plate. The resulting plate was incubated at 37° C. for 6 days. On day 9 post-editing cells were collected for flow cytometry analysis and NGS sequencing.

### Example 15.3. Flow Cytometry and NGS Sequencing

On day 9 post-editing, T cells were phenotyped by flow cytometry to determine CD3 protein expression as described in Example 6 using antibodies targeting CD3 (BioLegend® Cat. No. 317322) and Isotype Control-PE (BioLegend® Cat. No. 400269). DNA samples were subjected to PCR and subsequent NGS analysis, as described in Example 1. The mean percentage of each editing type at the TRAC locus and the mean number of CD3 negative cells after editing with BC22 and UGI are showing in Table 39; results after editing with Cas9 and UGI are shown in Table 40. C-to-T editing purity is calculated as the percentage of edited reads containing only C-to-T conversions.

TABLE 39
Percent editing and percent of CD3 negative cells following TRBC editing
with BC22

| | | | | | | | % C to T | | | | | |
|---|---|---|---|---|---|---|---|---|---|---|---|---|
| | % C to T | | % A to G | | % Indel | | purity | | | % CD3 negative | | |
| Guide | Mean | SD | Mean | SD | Mean | SD | Mean | SD | n | Mean | SD | N |
| G013015 | 93.67 | 0.06 | 3.16 | 0.19 | 2.46 | 0.19 | 94.34 | 0.00 | 2 | 32.55 | 1.77 | 2 |
| G014832 | 90.99 | 0.09 | 3.08 | 0.24 | 3.66 | 0.49 | 93.10 | 0.23 | 2 | 13.80 | 0.85 | 2 |
| G016167 | 88.56 | 0.62 | 5.06 | 0.26 | 5.76 | 0.47 | 89.11 | 0.72 | 2 | 30.25 | 7.28 | 2 |
| G016168 | 93.21 | 0.42 | 2.12 | 0.09 | 4.19 | 0.09 | 93.67 | 0.20 | 2 | 35.75 | 0.07 | 2 |
| G016171 | 92.69 | 0.50 | 3.59 | 0.15 | 3.09 | 0.33 | 93.28 | 0.48 | 2 | 57.90 | 0.28 | 2 |
| G016172 | 81.81 | 1.17 | 5.54 | 0.28 | 10.17 | 1.74 | 83.90 | 1.93 | 2 | 50.70 | 2.83 | 2 |
| G016174 | 92.42 | 1.66 | 0.99 | 0.09 | 4.74 | 0.19 | 94.17 | 0.20 | 2 | 92.75 | 0.78 | 2 |
| G016177 | 96.72 | 0.07 | 0.91 | 0.05 | 1.64 | 0.18 | 97.43 | 0.13 | 2 | 21.10 | 4.67 | 2 |
| G016179 | 82.05 | 1.68 | 6.67 | 0.28 | 9.58 | 0.83 | 83.46 | 1.22 | 2 | 40.00 | 2.12 | 2 |
| G016180 | 94.32 | 0.31 | 1.46 | 0.22 | 1.06 | 0.23 | 97.39 | 0.01 | 2 | 91.70 | 0.71 | 2 |
| G016181 | 92.19 | 0.51 | 3.77 | 0.30 | 2.04 | 0.14 | 94.07 | 0.18 | 2 | 19.20 | 1.27 | 2 |
| G016183 | 92.38 | 0.73 | 2.99 | 0.31 | 3.42 | 0.43 | 93.51 | 0.75 | 2 | 24.60 | 0.85 | 2 |
| G016187 | 92.08 | 2.19 | 3.14 | 0.33 | 1.88 | 0.47 | 94.82 | 0.90 | 2 | 13.90 | 0.85 | 2 |
| G016188 | 79.29 | 2.46 | 2.78 | 0.00 | 3.06 | 0.15 | 93.14 | 0.03 | 2 | 90.00 | 0.57 | 2 |
| G016189 | 93.26 | 0.49 | 2.79 | 0.19 | 2.76 | 0.45 | 94.38 | 0.64 | 2 | 92.05 | 0.64 | 2 |
| G016190 | 87.95 | 0.88 | 2.56 | 0.19 | 3.41 | 0.14 | 93.64 | 0.11 | 2 | 5.44 | 0.35 | 2 |
| G016191 | 51.28 | 4.28 | 2.37 | 0.21 | 2.65 | 0.05 | 91.06 | 0.41 | 2 | 3.88 | 0.59 | 2 |
| G016192 | 90.50 | 1.28 | 3.62 | 0.10 | 5.08 | 1.13 | 91.23 | 1.24 | 2 | 14.10 | 2.26 | 2 |
| G016193 | 88.60 | 0.69 | 3.61 | 0.10 | 4.95 | 0.05 | 91.19 | 0.11 | 2 | 8.06 | 0.46 | 2 |
| G016194 | 88.95 | 0.62 | 0.67 | 0.02 | 4.55 | 0.47 | 94.45 | 0.49 | 2 | 19.70 | 4.81 | 2 |
| G016195 | 91.17 | 0.74 | 2.57 | 0.15 | 3.19 | 0.23 | 94.05 | 0.04 | 2 | 13.65 | 1.91 | 2 |
| G016196 | 91.58 | 0.80 | 2.16 | 0.32 | 5.05 | 0.11 | 92.70 | 0.46 | 2 | 18.25 | 0.78 | 2 |
| G016197 | 89.55 | 0.90 | 2.72 | 0.17 | 6.99 | 0.68 | 90.22 | 0.87 | 2 | 16.25 | 7.71 | 2 |
| G016199 | 91.01 | 1.34 | 4.05 | 0.15 | 3.10 | 0.53 | 92.71 | 0.75 | 2 | 93.75 | 0.21 | 2 |
| G016200 | 94.68 | 0.86 | 2.09 | 0.34 | 2.68 | 0.60 | 95.20 | 0.95 | 2 | 93.95 | 0.07 | 2 |
| G016201 | 89.27 | 0.55 | 5.04 | 0.63 | 4.80 | 0.14 | 90.07 | 0.76 | 2 | 19.40 | 2.97 | 2 |
| G016203 | 79.59 | 1.09 | 3.78 | 0.55 | 9.40 | 0.25 | 85.80 | 0.57 | 2 | 12.95 | 0.49 | 2 |
| G016204 | 93.89 | 0.26 | 3.36 | 0.07 | 2.06 | 0.08 | 94.54 | 0.01 | 2 | 42.85 | 1.06 | 2 |
| G016205 | 67.12 | 3.18 | 3.84 | 0.00 | 5.66 | 0.09 | 87.59 | 0.62 | 2 | 41.35 | 1.48 | 2 |
| G016206 | 92.29 | 0.93 | 2.63 | 0.42 | 1.65 | 0.12 | 95.56 | 0.34 | 2 | 93.00 | 0.28 | 2 |
| G016207 | 85.70 | 2.33 | 5.16 | 0.32 | 8.56 | 2.71 | 86.20 | 2.39 | 2 | 44.60 | 0.71 | 2 |
| G016211 | 84.04 | 2.44 | 1.20 | 0.09 | 2.92 | 0.02 | 95.32 | 0.06 | 2 | 67.50 | 3.96 | 2 |
| G016212 | 95.39 | 0.87 | 0.78 | 0.11 | 2.11 | 0.44 | 97.05 | 0.58 | 2 | 48.30 | 0.00 | 2 |
| G016214 | 88.50 | 3.77 | 2.03 | 0.05 | 1.53 | 0.38 | 96.12 | 0.51 | 2 | 83.75 | 3.75 | 2 |
| G016216 | 91.37 | 0.45 | 3.67 | 0.40 | 4.65 | 0.84 | 91.65 | 0.44 | 2 | 60.65 | 0.92 | 2 |
| G016217 | 90.89 | 0.05 | 3.59 | 0.01 | 5.13 | 0.04 | 91.25 | 0.05 | 2 | 35.75 | 3.46 | 2 |
| G016219 | 89.15 | 1.07 | 3.93 | 0.16 | 5.59 | 0.64 | 90.35 | 0.83 | 2 | 47.85 | 2.33 | 2 |
| G016222 | 82.02 | 0.15 | 4.25 | 0.09 | 12.30 | 0.07 | 83.21 | 0.05 | 2 | 22.55 | 0.35 | 2 |
| G016227 | 67.64 | 0.78 | 4.66 | 0.28 | 14.36 | 0.70 | 78.05 | 0.57 | 2 | 38.20 | 1.27 | 2 |
| G016228 | 93.12 | 1.35 | 4.09 | 0.32 | 1.11 | 0.13 | 94.71 | 0.11 | 2 | 14.30 | 0.99 | 2 |
| G016230 | 88.34 | 1.74 | 3.06 | 0.11 | 5.98 | 1.48 | 90.71 | 1.45 | 2 | 13.25 | 2.76 | 2 |
| G016232 | 71.60 | n/a | 6.32 | n/a | 21.40 | n/a | 72.09 | n/a | 1 | 39.80 | 0.71 | 2 |
| G016233 | 87.08 | 1.67 | 2.87 | 0.18 | 8.87 | 2.03 | 88.13 | 1.86 | 2 | 22.20 | 8.91 | 2 |
| G016238 | 90.49 | 1.00 | 2.74 | 0.20 | 4.68 | 0.22 | 92.41 | 0.10 | 2 | 25.10 | 6.79 | 2 |
| G016239 | 87.37 | 0.96 | 5.11 | 0.06 | 7.02 | 1.07 | 87.81 | 1.01 | 2 | 29.95 | 1.63 | 2 |
| G016240 | 76.58 | 1.58 | 3.86 | 0.23 | 16.03 | 0.07 | 79.39 | 0.21 | 2 | 55.55 | 3.32 | 2 |
| G016241 | 69.44 | 1.35 | 3.89 | 0.04 | 24.19 | 1.07 | 71.21 | 1.21 | 2 | 61.50 | 1.84 | 2 |
| G016242 | 91.60 | 0.84 | 2.92 | 0.06 | 4.83 | 0.56 | 92.20 | 0.64 | 2 | 3.37 | 0.28 | 2 |
| G016243 | 93.32 | 2.21 | 2.74 | 0.02 | 1.69 | 0.36 | 95.46 | 0.48 | 2 | 33.75 | 5.16 | 2 |
| G016245 | 81.87 | 0.70 | 6.03 | 0.31 | 8.96 | 0.11 | 84.52 | 0.28 | 2 | 40.00 | 2.40 | 2 |
| G016248 | 81.23 | 2.35 | 1.14 | 0.04 | 2.95 | 0.34 | 95.21 | 0.20 | 2 | 5.33 | 0.21 | 2 |
| G016249 | No data | | | | | | | | | 6.87 | 2.02 | 2 |
| G016250 | No data | | | | | | | | | 12.50 | 2.12 | 2 |
| G016251 | 92.57 | 0.21 | 2.66 | 0.37 | 4.40 | 0.21 | 92.91 | 0.16 | 2 | 31.45 | 1.48 | 2 |
| G016252 | 84.54 | 0.88 | 1.23 | 0.06 | 1.93 | 0.03 | 96.40 | 0.14 | 2 | 2.47 | 0.17 | 2 |
| G016253 | 93.22 | 0.40 | 1.19 | 0.00 | 2.83 | 0.38 | 95.86 | 0.35 | 2 | 28.55 | 0.21 | 2 |
| G016254 | 92.52 | 0.86 | 2.96 | 0.16 | 3.53 | 0.57 | 93.45 | 0.75 | 2 | 2.02 | 0.44 | 2 |
| G016255 | 75.91 | 0.26 | 3.64 | 0.06 | 18.80 | 0.10 | 77.19 | 0.06 | 2 | 21.70 | 0.42 | 2 |
| G016256 | 69.87 | 5.75 | 3.53 | 0.29 | 1.87 | 0.48 | 92.82 | 0.31 | 2 | 6.39 | 2.44 | 2 |
| G016257 | 90.90 | 0.20 | 2.36 | 0.05 | 5.81 | 0.08 | 91.75 | 0.05 | 2 | 5.01 | 0.46 | 2 |
| G016258 | 69.09 | 1.72 | 3.05 | 0.31 | 24.53 | 0.99 | 71.46 | 1.01 | 2 | 26.90 | 0.71 | 2 |
| G016259 | 88.91 | 1.19 | 1.99 | 0.29 | 5.58 | 0.45 | 92.15 | 0.81 | 2 | 8.44 | 0.88 | 2 |
| G016260 | 92.85 | 1.13 | 1.82 | 0.27 | 2.58 | 0.11 | 95.48 | 0.42 | 2 | 4.30 | 0.07 | 2 |
| G016261 | 92.78 | 1.36 | 0.94 | 0.43 | 4.51 | 0.44 | 94.45 | 0.91 | 2 | 16.55 | 1.34 | 2 |
| G016262 | 95.60 | 0.22 | 1.36 | 0.03 | 2.59 | 0.37 | 96.03 | 0.34 | 2 | 4.29 | 0.81 | 2 |
| G016263 | No data | | | | | | | | | 78.40 | 0.14 | 2 |
| G016264 | 87.24 | 0.43 | 1.60 | 0.02 | 5.83 | 0.44 | 92.15 | 0.49 | 2 | 21.90 | 0.42 | 2 |
| G016265 | 48.92 | 6.40 | 2.49 | 0.13 | 1.49 | 0.07 | 92.41 | 0.81 | 2 | 2.53 | 0.22 | 2 |
| G016266 | 72.19 | 0.15 | 6.56 | 0.20 | 20.76 | 0.17 | 72.55 | 0.02 | 2 | 18.90 | 3.68 | 2 |
| G016267 | 92.15 | 0.40 | 4.24 | 0.38 | 2.94 | 0.26 | 92.78 | 0.14 | 2 | 16.65 | 8.70 | 2 |
| G016268 | 93.60 | 0.39 | 3.72 | 0.25 | 2.43 | 0.04 | 93.83 | 0.29 | 2 | 3.03 | 0.24 | 2 |
| G016269 | 48.11 | 0.16 | 4.00 | 0.55 | 2.93 | 0.20 | 87.42 | 1.23 | 2 | 17.05 | 1.63 | 2 |
| G016270 | 87.48 | 0.28 | 2.81 | 0.16 | 9.04 | 0.32 | 88.06 | 0.46 | 2 | 66.90 | 0.57 | 2 |
| G016271 | 91.19 | 1.41 | 4.00 | 0.08 | 3.94 | 1.08 | 91.99 | 1.19 | 2 | 14.50 | 10.33 | 2 |
| G016272 | 93.90 | 0.09 | 1.39 | 0.12 | 4.07 | 0.23 | 94.50 | 0.11 | 2 | 27.20 | 1.56 | 2 |
| G016273 | 87.75 | 0.20 | 5.66 | 0.09 | 5.36 | 0.36 | 88.84 | 0.26 | 2 | 31.50 | 3.25 | 2 |
| G016274 | 83.68 | 1.26 | 4.02 | 0.19 | 7.12 | 0.27 | 88.24 | 0.59 | 2 | 17.50 | 6.93 | 2 |
| G016275 | 93.35 | 0.24 | 3.14 | 0.20 | 2.52 | 0.19 | 94.28 | 0.00 | 2 | 14.65 | 9.26 | 2 |
| G016276 | 94.95 | 0.27 | 1.98 | 0.04 | 2.65 | 0.21 | 95.35 | 0.26 | 2 | 30.35 | 1.48 | 2 |
| G016277 | 90.15 | 1.29 | 5.09 | 0.18 | 1.82 | 0.65 | 92.89 | 0.54 | 2 | 16.13 | 13.68 | 2 |
| G016278 | 83.20 | 3.62 | 5.41 | 0.96 | 2.92 | 0.11 | 90.88 | 1.20 | 2 | 8.88 | 1.59 | 2 |
| G016279 | 90.24 | 0.72 | 4.88 | 0.45 | 3.23 | 0.08 | 91.76 | 0.40 | 2 | 13.70 | 5.23 | 2 |
| G016280 | 91.06 | 1.61 | 2.95 | 0.43 | 2.76 | 0.45 | 94.09 | 0.95 | 2 | 13.81 | 7.06 | 2 |
| G016281 | 94.85 | 0.41 | 1.28 | 0.01 | 3.31 | 0.47 | 95.38 | 0.46 | 2 | 13.85 | 5.45 | 2 |
| G016282 | 93.39 | 0.27 | 3.06 | 0.05 | 3.13 | 0.18 | 93.78 | 0.24 | 2 | 16.95 | 7.00 | 2 |
| G016283 | 77.09 | n/a | 5.72 | n/a | 16.13 | n/a | 77.92 | n/a | 1 | 20.40 | 3.25 | 2 |
| G016284 | 87.29 | 1.73 | 2.01 | 1.11 | 9.08 | 0.55 | 89.29 | 2.50 | 2 | 8.38 | 1.51 | 2 |
| G016285 | 60.72 | 3.51 | 8.71 | 0.53 | 13.95 | 2.51 | 72.80 | 2.87 | 2 | 15.70 | 2.12 | 2 |

TABLE 40
Percent editing and percent CD3 negative cells following TRBC editing with
Cas9

| | % C to T | | | % A to G | | | % Indel | | | % CD3 negative | | |
|---|---|---|---|---|---|---|---|---|---|---|---|---|
| Guide | Mean | SD | n | Mean | SD | n | Mean | SD | n | Mean | SD | N |
| G013015 | 0.03 | 0.04 | 2 | 0.09 | 0.01 | 2 | 99.43 | 0.14 | 2 | 93.60 | 0.28 | 2 |
| G014832 | 0.07 | 0.02 | 2 | 0.10 | 0.00 | 2 | 98.24 | 0.65 | 2 | 84.95 | 0.07 | 2 |
| G016167 | 0.01 | 0.00 | 2 | 0.00 | 0.00 | 2 | 99.36 | 0.26 | 2 | 90.10 | 0.28 | 2 |
| G016168 | 0.02 | 0.01 | 2 | 0.18 | 0.14 | 2 | 98.29 | 0.07 | 2 | 95.00 | 0.28 | 2 |
| G016171 | 0.03 | 0.04 | 2 | 0.03 | 0.02 | 2 | 99.11 | 0.34 | 2 | 91.80 | 0.71 | 2 |
| G016172 | 0.01 | 0.00 | 2 | 1.29 | 0.45 | 2 | 96.54 | 1.15 | 2 | 93.60 | 0.42 | 2 |
| G016174 | 0.05 | 0.05 | 2 | 0.14 | 0.05 | 2 | 98.65 | 0.68 | 2 | 94.10 | 0.42 | 2 |
| G016177 | 0.10 | 0.03 | 2 | 0.08 | 0.03 | 2 | 99.02 | 0.28 | 2 | 94.05 | 0.35 | 2 |
| G016179 | 0.09 | 0.02 | 2 | 0.21 | 0.04 | 2 | 97.55 | 0.18 | 2 | 93.20 | 0.42 | 2 |
| G016180 | 0.01 | 0.00 | 2 | 0.19 | 0.07 | 2 | 95.64 | 0.93 | 2 | 91.25 | 1.20 | 2 |
| G016181 | 0.25 | | 1 | 0.03 | | 1 | 94.84 | | 1 | 92.00 | 0.28 | 2 |
| G016183 | 0.04 | 0.04 | 2 | 0.01 | 0.01 | 2 | 98.56 | 0.53 | 2 | 92.90 | 0.00 | 2 |
| G016187 | 0.18 | 0.00 | 2 | 0.05 | 0.07 | 2 | 99.07 | 0.07 | 2 | 93.95 | 0.64 | 2 |
| G016188 | 0.07 | 0.03 | 2 | 0.30 | 0.07 | 2 | 94.24 | 0.69 | 2 | 85.95 | 0.35 | 2 |
| G016189 | 0.12 | 0.03 | 2 | 0.64 | 0.21 | 2 | 97.19 | 0.73 | 2 | 89.75 | 0.49 | 2 |
| G016190 | 6.51 | 6.05 | 2 | 0.15 | 0.21 | 2 | 77.21 | 4.03 | 2 | 70.55 | 0.35 | 2 |
| G016191 | 3.67 | 0.17 | 2 | 0.58 | 0.13 | 2 | 57.71 | 0.58 | 2 | 63.95 | 0.21 | 2 |
| G016192 | 0.11 | 0.08 | 2 | 0.24 | 0.09 | 2 | 98.50 | 0.20 | 2 | 89.50 | 0.28 | 2 |
| G016193 | 0.05 | 0.02 | 2 | 0.15 | 0.04 | 2 | 98.54 | 0.99 | 2 | 92.45 | 0.35 | 2 |
| G016194 | 4.24 | 0.49 | 2 | 0.06 | 0.01 | 2 | 85.43 | 0.76 | 2 | 68.00 | 0.14 | 2 |
| G016195 | 0.03 | 0.04 | 2 | 0.00 | 0.00 | 2 | 99.51 | 0.20 | 2 | 93.55 | 0.35 | 2 |
| G016196 | 0.06 | 0.01 | 2 | 0.15 | 0.05 | 2 | 80.55 | 2.75 | 2 | 82.15 | 1.34 | 2 |
| G016197 | 0.00 | 0.01 | 2 | 0.01 | 0.00 | 2 | 99.64 | 0.02 | 2 | 75.20 | 0.42 | 2 |
| G016199 | 0.15 | 0.01 | 2 | 0.22 | 0.01 | 2 | 98.25 | 0.19 | 2 | 91.70 | 0.57 | 2 |
| G016200 | 0.01 | 0.01 | 2 | 0.06 | 0.02 | 2 | 99.45 | 0.16 | 2 | 93.65 | 0.64 | 2 |
| G016201 | 0.05 | 0.05 | 2 | 0.15 | 0.00 | 2 | 93.38 | 0.20 | 2 | 85.10 | 0.71 | 2 |
| G016203 | 0.05 | n/a | 1 | 0.09 | n/a | 1 | 98.61 | n/a | 1 | 92.20 | 0.28 | 2 |
| G016204 | 0.08 | 0.02 | 2 | 0.02 | 0.01 | 2 | 99.31 | 0.23 | 2 | 92.50 | 0.57 | 2 |
| G016205 | 0.05 | 0.04 | 2 | 0.75 | 0.02 | 2 | 77.14 | 2.06 | 2 | 76.20 | 2.69 | 2 |
| G016206 | 0.24 | 0.09 | 2 | 0.24 | 0.05 | 2 | 96.55 | 0.05 | 2 | 91.85 | 0.07 | 2 |
| G016207 | 0.06 | 0.03 | 2 | 0.07 | 0.01 | 2 | 99.25 | 0.02 | 2 | 93.80 | 0.42 | 2 |
| G016211 | 0.04 | 0.02 | 2 | 0.09 | 0.01 | 2 | 84.26 | 0.46 | 2 | 87.75 | 1.34 | 2 |
| G016212 | 0.09 | 0.00 | 2 | 0.10 | 0.08 | 2 | 91.57 | 1.87 | 2 | 90.90 | 0.42 | 2 |
| G016214 | 0.07 | 0.05 | 2 | 0.16 | 0.02 | 2 | 88.68 | 0.74 | 2 | 87.10 | 0.85 | 2 |
| G016216 | 0.08 | 0.06 | 2 | 0.83 | 0.28 | 2 | 98.49 | 0.46 | 2 | 88.50 | 0.85 | 2 |
| G016217 | 0.17 | 0.06 | 2 | 0.08 | 0.01 | 2 | 98.85 | 0.25 | 2 | 93.70 | 0.85 | 2 |
| G016219 | 0.15 | 0.21 | 2 | 0.08 | 0.04 | 2 | 97.51 | 0.98 | 2 | 89.95 | 0.92 | 2 |
| G016222 | 0.03 | 0.02 | 2 | 0.08 | 0.03 | 2 | 98.09 | 0.23 | 2 | 93.35 | 0.35 | 2 |
| G016227 | 0.02 | n/a | 1 | 0.12 | n/a | 1 | 98.97 | n/a | 1 | 84.30 | 0.71 | 2 |
| G016228 | 0.12 | 0.17 | 2 | 0.10 | 0.08 | 2 | 99.05 | 0.21 | 2 | 92.65 | 0.21 | 2 |
| G016230 | 0.10 | 0.00 | 2 | 0.04 | 0.02 | 2 | 98.96 | 0.30 | 2 | 77.90 | 0.42 | 2 |
| G016232 | 0.13 | 0.04 | 1 | 0.02 | 0.01 | 1 | 99.07 | 0.51 | 1 | 94.45 | 0.21 | 2 |
| G016233 | 0.07 | 0.01 | 2 | 0.07 | 0.04 | 2 | 99.00 | 0.45 | 2 | 71.00 | 1.27 | 2 |
| G016238 | 0.09 | 0.08 | 2 | 0.14 | 0.02 | 2 | 93.50 | 0.81 | 2 | 90.90 | 0.00 | 2 |
| G016239 | 0.08 | 0.08 | 2 | 0.09 | 0.05 | 2 | 98.74 | 0.30 | 2 | 85.70 | 0.71 | 2 |
| G016240 | 0.00 | n/a | 1 | 0.05 | n/a | 1 | 98.91 | n/a | 1 | 84.50 | 0.28 | 2 |
| G016241 | 0.00 | 0.00 | 2 | 0.00 | 0.00 | 2 | 99.56 | 0.05 | 2 | 86.05 | 0.35 | 2 |
| G016242 | 0.00 | 0.00 | 2 | 0.03 | 0.03 | 2 | 98.50 | 0.02 | 2 | 87.55 | 0.64 | 2 |
| G016243 | 0.00 | 0.00 | 2 | 0.07 | 0.01 | 2 | 98.44 | 0.49 | 2 | 93.55 | 0.78 | 2 |
| G016245 | 0.45 | 0.51 | 2 | 2.10 | 0.20 | 2 | 95.23 | 1.04 | 2 | 92.70 | 0.14 | 2 |
| G016248 | 2.51 | 0.44 | 2 | 0.09 | 0.03 | 2 | 85.91 | 2.49 | 2 | 78.90 | 1.13 | 2 |
| G016249 | 0.00 | 0.00 | 0 | 0.01 | 0.00 | 0 | 98.87 | 0.42 | 0 | 93.05 | 0.07 | 2 |
| G016250 | 0.00 | 0.00 | 0 | 0.01 | 0.00 | 0 | 99.11 | 0.30 | 0 | 93.35 | 1.06 | 2 |
| G016251 | 0.01 | 0.02 | 2 | 0.04 | 0.02 | 2 | 97.64 | 1.98 | 2 | 93.40 | 0.42 | 2 |
| G016252 | 0.01 | 0.01 | 2 | 0.05 | 0.01 | 2 | 76.81 | 1.22 | 2 | 28.95 | 3.75 | 2 |
| G016253 | 0.11 | 0.08 | 2 | 0.06 | 0.05 | 2 | 95.66 | 0.25 | 2 | 67.70 | 0.71 | 2 |
| G016254 | 0.05 | 0.02 | 2 | 0.07 | 0.01 | 2 | 98.21 | 0.25 | 2 | 25.65 | 0.92 | 2 |
| G016255 | 0.01 | 0.01 | 2 | 1.08 | 1.33 | 2 | 97.81 | 0.73 | 2 | 62.75 | 0.07 | 2 |
| G016256 | 0.06 | 0.01 | 2 | 0.46 | 0.07 | 2 | 80.84 | 1.58 | 2 | 21.05 | 8.41 | 2 |
| G016257 | 0.03 | 0.02 | 2 | 0.01 | 0.00 | 2 | 97.59 | 0.32 | 2 | 85.70 | 0.00 | 2 |
| G016258 | 0.10 | 0.13 | 2 | 0.23 | 0.04 | 2 | 78.74 | 3.06 | 2 | 47.45 | 1.20 | 2 |
| G016259 | 0.00 | 0.00 | 2 | 0.21 | 0.04 | 2 | 97.82 | 0.31 | 2 | 47.00 | 2.12 | 2 |
| G016260 | 1.16 | 0.20 | 2 | 0.15 | 0.15 | 2 | 94.72 | 0.27 | 2 | 28.30 | 0.28 | 2 |
| G016261 | 0.03 | 0.04 | 2 | 0.03 | 0.04 | 2 | 95.13 | 0.46 | 2 | 90.95 | 0.07 | 2 |
| G016262 | 1.17 | 0.31 | 2 | 0.07 | 0.05 | 2 | 98.39 | 0.31 | 2 | 26.85 | 0.49 | 2 |
| G016263 | 0.05 | 0.06 | 0 | 0.08 | 0.07 | 0 | 97.11 | 2.82 | 0 | 93.50 | 0.28 | 2 |
| G016264 | 0.09 | 0.01 | 2 | 0.14 | 0.04 | 2 | 75.23 | 0.49 | 2 | 48.70 | 1.70 | 2 |
| G016265 | 0.08 | 0.01 | 2 | 0.29 | 0.01 | 2 | 47.59 | 0.92 | 2 | 15.60 | 1.13 | 2 |
| G016266 | 0.02 | 0.00 | 2 | 16.72 | 0.36 | 2 | 82.14 | 0.85 | 2 | 25.25 | 2.76 | 2 |
| G016267 | 0.09 | 0.01 | 2 | 0.15 | 0.06 | 2 | 97.50 | 0.21 | 2 | 34.20 | 1.56 | 2 |
| G016268 | 0.01 | 0.00 | 2 | 0.07 | 0.00 | 2 | 98.95 | 0.20 | 2 | 40.30 | 1.41 | 2 |
| G016269 | 0.14 | 0.06 | 2 | 0.65 | 0.01 | 2 | 38.34 | 4.64 | 2 | 15.35 | 0.64 | 2 |
| G016270 | 0.12 | 0.07 | 2 | 0.32 | 0.13 | 2 | 99.10 | 0.33 | 2 | 93.60 | 0.85 | 2 |
| G016271 | 0.16 | 0.04 | 2 | 0.10 | 0.01 | 2 | 98.21 | 0.27 | 2 | 35.55 | 5.16 | 2 |
| G016272 | 0.09 | 0.05 | 2 | 0.19 | 0.04 | 2 | 94.63 | 1.28 | 2 | 93.45 | 0.78 | 2 |
| G016273 | 0.00 | 0.00 | 2 | 0.01 | 0.00 | 2 | 97.97 | 0.37 | 2 | 92.85 | 1.20 | 2 |
| G016274 | 0.06 | 0.02 | 2 | 0.23 | 0.12 | 2 | 87.53 | 0.83 | 2 | 71.30 | 1.84 | 2 |
| G016275 | 0.24 | 0.22 | 2 | 0.03 | 0.00 | 2 | 98.58 | 0.13 | 2 | 31.50 | 2.97 | 2 |
| G016276 | 0.02 | 0.01 | 2 | 0.34 | 0.04 | 2 | 98.56 | 0.14 | 2 | 24.35 | 4.03 | 2 |
| G016277 | 0.52 | 0.04 | 2 | 0.32 | 0.04 | 2 | 97.86 | 0.06 | 2 | 31.75 | 4.31 | 2 |
| G016278 | 0.30 | 0.01 | 2 | 0.27 | 0.04 | 2 | 98.12 | 0.07 | 2 | 32.10 | 0.28 | 2 |
| G016279 | 0.59 | 0.15 | 2 | 0.14 | 0.00 | 2 | 98.69 | 0.12 | 2 | 30.20 | 5.37 | 2 |
| G016280 | 0.24 | 0.10 | 2 | 0.24 | 0.01 | 2 | 90.41 | 0.60 | 2 | 28.55 | 3.46 | 2 |
| G016281 | 0.65 | 0.88 | 2 | 0.03 | 0.04 | 2 | 98.14 | 0.28 | 2 | 92.90 | 0.42 | 2 |
| G016282 | 0.04 | 0.02 | 2 | 0.01 | 0.00 | 2 | 98.57 | 0.05 | 2 | 93.45 | 0.21 | 2 |
| G016283 | 0.01 | n/a | 1 | 0.01 | n/a | 1 | 93.16 | n/a | 1 | 87.35 | 2.47 | 2 |
| G016284 | 0.02 | 0.02 | 2 | 0.01 | 0.00 | 2 | 97.97 | 0.35 | 2 | 42.05 | 0.64 | 2 |
| G016285 | 0.06 | 0.08 | 2 | 0.01 | 0.01 | 2 | 97.89 | 0.86 | 2 | 89.80 | 0.14 | 2 |

### Example 16. Profiling RNA Off-Targets Following 1D2M Editing

### Example 16.1. T Cell Preparation

Healthy human donor apheresis was obtained commercially (Hemacare), and cells were washed and re-suspended in CliniMACS® PBS/EDTA buffer (Miltenyi Biotec Cat. No. 130-070-525) on the LOVO device. T cells were isolated via positive selection using CD4 and CD8 magnetic beads (Miltenyi Biotec Cat. No. 130-030-401/130-030-801) using the CliniMACS®) Plus and CliniMACS®) LS disposable kit. T cells were aliquoted into vials and cryopreserved in Cryostor® CS10 (StemCell Technologies Cat. 07930) for future use. Upon thawing, T cells were plated at a density of 1.0×10\hat{ }6 cells/nmL in T cell growth (TCG) media consisting of CTS OpTmiizer T cell expansion serum-free media (Thermofisher, Cat. A3705001) supplemented with 5% human AB serum (Gemini, Cat. 100-512), 1× GlutaMAX (Thermofisher, Cat. 35050061), 10 mM HEPES (Thermofisher, Cat. 15630080) and 1× of Penicillin-Streptomycin, further supplemented with 200 U/mL IL-2 (Peprotech, Cat. 200-02), 10 ng/ml IL-7 (Peprotech, Cat. 200-07), 10 ng/ml IL-15 (Peprotech, Cat. 200-15). T cells were activated with TransAct™ (1:100 dilution, Miltenyi Biotec, Cat. 130-111-160). Cells were expanded for 72 hours at 37° C. prior to mRNA electroporation.

### Example 16.2. mRNA and sgRNA Electroporation of T Cells

A solution containing mRNA encoding Cas9 (SEQ ID NO: 11), BC22n (SEQ ID NO: 2), UGI (SEQ ID NO: 26) or BE4MAX (SEQ ID NO: 32) was prepared in sterile water. A 50 μM B2M targeting sgRNA (G015995) was removed from its storage plate and denatured for 2 minutes at 95° C. before cooling at room temperature. Seventy-two hours post activation, T cells were harvested, centrifuged, and resuspended at a concentration of 12.5×10\hat{ }6 T cells/mL in P3 electroporation buffer (Lonza). For each well to be electroporated, 1×10\hat{ }5 T cells were mixed with 200 ng of mRNAs and 20 pmols of sgRNA in a final volume of 20 uL of P3 electroporation buffer. The T cell mix was transferred in 5 replicates to a 96-well Nucleofector™ plate and electroporated using the manufacturer's pulse code. Electroporated T cells were immediately rested in 80 μL of TCG media without cytokines for 15 minutes before being transferred to a new flat-bottom 96-well plate containing an additional 100 μL of TCG media supplemented with 2× cytokines cited in Section 16.1.

To evaluate RNA off-targets when expression level of BC22n peaks, a fraction of edited T cells was collected 24 h post-electroporation. This fraction was further divided into 2 plates, one of which was subjected to cell lysis, PCR amplification and NGS analysis, while the other fraction was used for RNA extraction and transcriptome sequencing. On day 3 post-electroporation, the remaining T cells were collected for cell lysis and NGS sequencing, which enabled confirmation of maximum B2M editing in these samples at a timepoint when editing is normally complete.

### Example 16.3. NGS Sequencing

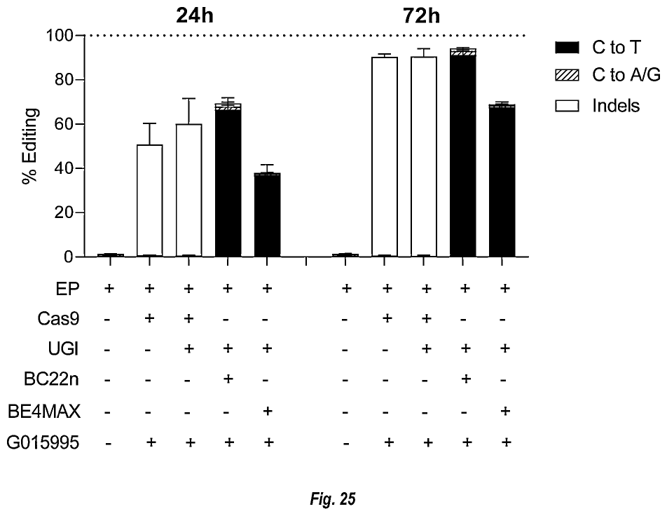

At 24 and 72 h post-electroporation, T cells were subjected to lysis, PCR amplification of the B2M locus and subsequent NGS analysis, as described in Example 1. Table 41 and FIG. 25 show B2M editing levels in T cells collected at both timepoints following B2M editing.

TABLE 41
Mean editing percentages at the B2M locus following editing

| | | | % C to T | | % C to A/G | | % Indel | |
|---|---|---|---|---|---|---|---|---|
| Time (h) | mRNA | sgRNA | Mean | SD | Mean | SD | Mean | SD |
| 24 | None | None | 0.58 | 0.06 | 0.60 | 0.05 | 0.22 | 0.01 |
| | Cas9 | G015995 | 0.48 | 0.27 | 0.35 | 0.05 | 49.97 | 9.52 |
| | Cas9, UGI | G015995 | 0.35 | 0.09 | 0.36 | 0.05 | 59.44 | 11.46 |
| | BC22n, UGI | G015995 | 66.57 | 5.39 | 1.27 | 0.64 | 1.52 | 0.61 |
| | BE4MAX, UGI | G015995 | 36.83 | 4.89 | 0.70 | 0.10 | 0.38 | 0.19 |
| 72 | None | None | 0.60 | 0.09 | 0.61 | 0.06 | 0.23 | 0.06 |
| | Cas9 | G015995 | 0.33 | 0.31 | 0.28 | 0.16 | 89.74 | 1.38 |
| | Cas9, UGI | G015995 | 0.30 | 0.24 | 0.25 | 0.21 | 90.02 | 3.54 |
| | BC22n, UGI | G015995 | 91.19 | 1.80 | 1.84 | 0.64 | 1.19 | 0.37 |
| | BE4MAX, UGI | G015995 | 67.62 | 2.38 | 0.84 | 0.20 | 0.49 | 0.19 |

### Example 16.4. Whole Transcriptome Sequencing

At 24 h post-electroporation, T cells were centrifuged, and the cell pellet was resuspended in 200 uL of TRIzol™ reagent (Thermo Fisher Scientific, Cat No. 15596026) which was frozen at −80 C for future processing. Total RNA was extracted from samples in TRIzol™ reagent using the Direct-zol RNA microprep kit (Zymo Research, Cat No. R2062) following the manufacturer's protocol. Purified RNA samples were quantified in a NanoDrop™ 8000 spectrophotometer (Thermo Fisher Scientific) and diluted to 18.18 ng/uL using nuclease-free water. From each experimental group shown in FIG. 25, 3 samples were randomly chosen for transcriptomic analysis. From each sample, 100 ng (5.5 uL) of purified total RNA were depleted of ribosomal RNA (rRNA) components using the NEBNext® rRNA depletion kit (New England Biolabs, Cat. E6350L) according to the manufacturer's instructions. rRNA-depleted samples were converted into double-stranded DNA libraries using NEBNext® Ultra™ II directional RNA library prep kit for Illumina® (New England Biolabs, Cat. E7765S) following the manufacturer's protocol. Amplified libraries were quantified in a Qubit 4 fluorometer (Thermo Fisher Scientific) and the average fragment size of each library was obtained by capillary electrophoresis. Libraries were pooled at an equimolar concentration and sequenced in the Illumina NextSeq550 platform using a high-output 300-cycle kit (Illumina, Cat. 20024908).

### Example 16.5. Data Processing for Single Nucleotide Variant (SNV) Analysis

Paired-end reads were aligned to human genome GRCh38 with STAR v2.7.1a (Dobin et al., 2013). PCR duplicates were removed with Picard MarkDuplicates v2.19.0 (BroadInstitute, 2019). GATK tools SplitNCigarReads, BaseRecalibrator, ApplyBQSR v4.1.8.1 were deployed consecutively to preprocess alignments. Variations were called with GATK HaplotypeCaller (Auwera et al., 2013; DePristo et al., 2011; McKenna et al., 2010). Variants discovered from replicates of the same sample were merged using bcftools v1.8 (Li, 2011). Sample specific variants were retrieved by excluding variants discovered in controls using vcflib v1.0.0 (Garrison, 2016). Relative C to U frequencies were calculated by dividing the number of C to U variants by the total number of SNVs for each sample. Treatment groups were compared using unpaired t-tests and the statistical significance was determined using the Holm-Sidak method with an alpha of 0.05, without assuming a consistent standard deviation.

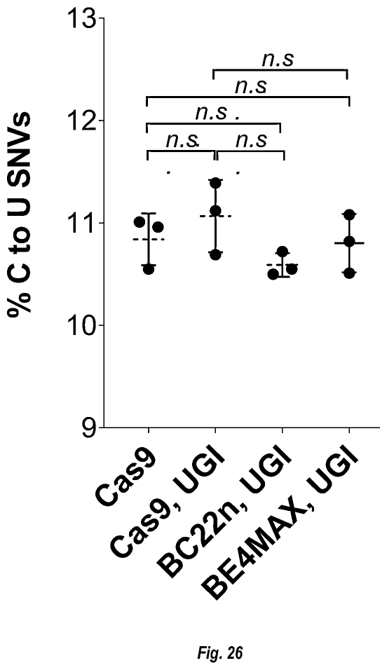

Compared to samples treated with Cas9 mRNA, T cells electroporated with both Cas9 and UGI mRNAs, BC22n and UGI mRNAs or BE4MAX and UGI mRNAs showed no statistically significant (p<0.05) increase in the frequency of C to U transitions, demonstrating the absence of detectable homology-independent cytosine deamination events in the transcriptome of these samples (Table 42 and FIG. 26).

TABLE 42
Mean percentage of SNVs in RNA transcripts that are C to U conversions

| | | % C to U SNVs | | | |
|---|---|---|---|---|---|
| | | | | p value | p value |
| mRNA(s) | sgRNA | Mean | SD | (vs. Cas9) | (vs. Cas9, UGI) |
| Cas9 | G015995 | 10.84 | 0.25 | NA | 0.42 |
| Cas9, UGI | G015995 | 11.07 | 0.35 | 0.42 | NA |
| BC22n, UGI | G015995 | 10.59 | 0.12 | 0.19 | 0.09 |
| BE4MAX, UGI | G015995 | 10.80 | 0.29 | 0.88 | 0.37 |

### Example 17. Whole Genome Sequencing of Amplified T Cell Genomes Following B2M Editing

### Example 17.1. T Cell Preparation

Healthy human donor apheresis was obtained commercially (Hemacare), and cells were washed and re-suspended in CliniMACS® PBS/EDTA buffer (Miltenyi Biotec Cat. No. 130-070-525) on the LOVO device. T cells were isolated via positive selection using CD4 and CD8 magnetic beads (Miltenyi Biotec Cat. No. 130-030-401/130-030-801) using the CliniMACS® Plus and CliniMACS® LS disposable kit. T cells were aliquoted into vials and cryopreserved in Cryostor® CS10 (StemCell Technologies Cat. 07930) for future use. Upon thawing, T cells were plated at a density of 1.0×10\hat{ }6 cells/mL in T cell growth (TCG) media consisting of CTS OpTmizer T cell expansion serum-free media (Thermofisher, Cat. A3705001) supplemented with 5% human AB serum (Gemini, Cat. 100-512), 1× GlutaMAX (Thermofisher, Cat. 35050061), 10 mM HEPES (Thermofisher, Cat. 15630080) and 1× of Penicillin-Streptomycin, further supplemented with 200 U/mL IL-2 (Peprotech, Cat. 200-02), 10 ng/ml IL-7 (Peprotech, Cat. 200-07), 10 ng/ml IL-15 (Peprotech, Cat. 200-15). T cells were activated with TransAct™ (1:100 dilution, Miltenyi Biotec, Cat. 130-111-160). Cells were expanded for 72 hours at 37° C. prior to mRNA electroporation.

### Example 17.2. mRNA and sgRNA Electroporation of T Cells

A solution containing mRNA encoding Cas9 (SEQ ID NO: 11), BC22n (SEQ ID NO: 2) or UGI (SEQ ID NO: 26) was prepared in sterile water. A 50 μM B2M targeting sgRNA (G015995) was removed from its storage plate and denatured for 2 minutes at 95° C. before cooling at room temperature. Seventy-two hours post activation, T cells were harvested, centrifuged, and resuspended at a concentration of 12.5×10^{\hat{ }6} T cells/mL in P3 electroporation buffer (Lonza). For each well to be electroporated, 1×10\hat{ }5 T cells were mixed with 200 ng of mRNAs and 20 pmols of sgRNA in a final volume of 20 uL of P3 electroporation buffer. The T cell mix was transferred in 8 replicates to a 96-well Nucleofector™ plate and electroporated using the manufacturer's pulse code. Electroporated T cells were immediately rested in 80 μL of TCG media without cytokines for 15 minutes before being transferred to a new flat-bottom 96-well plate containing an additional 100 IL of TCG media supplemented with 2× cytokines.

To promote expansion, T cells were split at the ratios of 1:4 and 1:3 on days 3 and 6 post-electroporation, respectively, using fresh TCG media with 1× cytokines. On day 7 post-electroporation, a fraction of cells was collected for flow cytometry and NGS sequencing, while the remaining cells were frozen for subsequent single-cell whole genome amplification and sequencing.

### Example 17.3. Flow Cytometry and NGS Sequencing

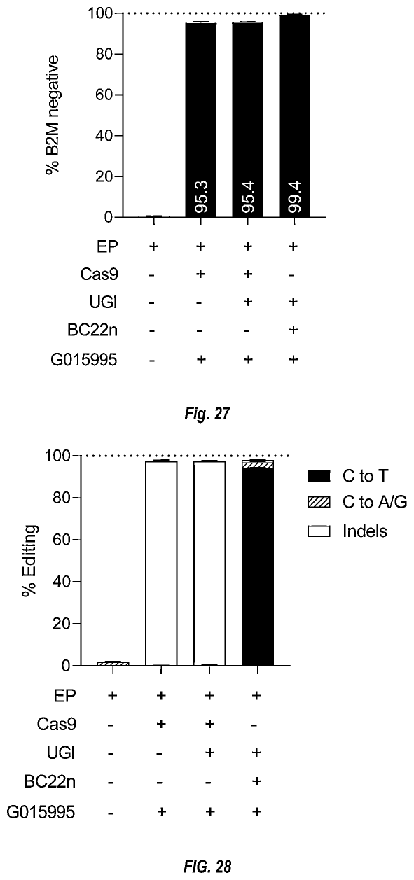

On day 7 post-electroporation, T cells were phenotyped by flow cytometry to evaluate loss of B2M expression levels following editing with sgRNA G015995. Briefly, T cells were incubated for 30 min at 4° C. with a mixture of antibodies against CD3 (BioLegend® Cat. No. 317340), CD4 (BioLegend® Cat. No. 300537), CD8 (BioLegend® Cat. No. 344706) and B2M (BioLegend® Cat. No. 316314), diluted at 1:200 in cell staining buffer (BioLegend® Cat. No. 420201). Cells were subsequently washed, processed on a Cytoflex flow cytometer (Beckman Coulter) and analyzed using the FlowJo software package. T cells were gated based on size, shape and B2M expression. Table 43 and FIG. 27 show the percentage of cells expressing B2M in edited T cells.

TABLE 43
Loss of B2M expression (% of cells lacking surface marker)
assessed by flow cytometry analysis of T cells harvested 7
days post-treatment with different mRNAcombinations
and B2M sgRNA G015995 (SD = standard deviation).

| | | | % B2M KO | |
|---|---|---|---|---|
| Group | sgRNA | mRNA(s) | Mean | SD |
| 1 | None | None | 0.36 | 0.32 |
| 2 | G015995 | Cas9 | 95.27 | 0.59 |
| 3 | G015995 | Cas9, UGI | 95.45 | 0.44 |
| 4 | G015995 | BC22n, UGI | 99.37 | 0.21 |

On day 7 post-electroporation, T cells were also subjected to lysis, PCR amplification of the B2M locus and subsequent NGS analysis, as described in Example 1. Table 44 and FIG. 28 show B2M editing levels in 32 samples (8 replicates per group) following B2M editing.

TABLE 44
Percent B2M editing of T cells harvested 7 days post-treatment
with different mRNA combinations and B2M sgRNA G015995
(Indels = insertions or deletions).

| Group | Sample | % C to T | % C to A/G | % Indels |
|---|---|---|---|---|
| 1- | 1 | 0.35 | 1.47 | 0.17 |
| Electroporation | 2 | 0.31 | 1.35 | 0.14 |
| only | 3 | 0.29 | 1.49 | 0.14 |
| | 4 | 0.29 | 1.48 | 0.18 |
| | 5 | 0.30 | 1.52 | 0.20 |
| | 6 | 0.27 | 1.51 | 0.21 |
| | 7 | 0.31 | 1.55 | 0.21 |
| | 8 | 0.31 | 1.57 | 0.21 |
| 2-Cas9 + | 9 | 0.08 | 0.10 | 96.96 |
| G015995 | 10 | 0.18 | 0.07 | 96.47 |
| | 11 | 0.18 | 0.08 | 97.57 |
| | 12 | 0.16 | 0.04 | 97.59 |
| | 13 | 0.16 | 0.06 | 97.53 |
| | 14 | 0.10 | 0.05 | 97.73 |
| | 15 | 0.43 | 0.11 | 96.12 |
| | 16 | 0.15 | 0.09 | 97.36 |
| 3-Cas9 + UGI + | 17 | 0.09 | 0.08 | 97.05 |
| G015995 | 18 | 0.42 | 0.17 | 96.70 |
| | 19 | 0.23 | 0.16 | 96.70 |
| | 20 | 0.28 | 0.04 | 97.20 |
| | 21 | 0.20 | 0.03 | 97.60 |
| | 22 | 0.06 | 0.09 | 97.14 |
| | 23 | 0.32 | 0.06 | 97.38 |
| | 24 | 0.16 | 0.05 | 97.36 |
| 4-BC22n + | 25 | 94.29 | 2.68 | 0.99 |
| UGI + G015995 | 26 | 93.74 | 3.25 | 1.19 |
| | 27 | 93.61 | 3.14 | 0.84 |
| | 28 | 93.94 | 2.93 | 1.01 |
| | 29 | 94.47 | 2.86 | 1.17 |
| | 30 | 94.55 | 2.53 | 1.45 |
| | 31 | 94.63 | 2.40 | 0.79 |
| | 32 | 94.14 | 2.79 | 0.78 |

### Example 17.4. Single T Cell Isolation, Lysis, Whole Genome Amplification and Sequencing

One sample from each group of 8 replicates was randomly chosen for single cell isolation, whole genome amplification and sequencing. Frozen cells from samples 12, 21 and 30 (Table 44) were transferred to a contract research organization (Singulomics Corporation, Inc.) where 10 single T cells were isolated from each sample. These single T cells were lysed, and their genomes were amplified using multiple displacement amplification (MDA), according to previously published methods (Dong et al., Nat Methods, 2017). Amplified genomes were subjected to PCR amplification of the B2M locus and subsequent NGS analysis, as described in Example 1, aiming to confirm the edited genotype in single T cells. From each group of 10 single cells, 6 DNA samples were converted into whole genome sequencing libraries using the KAPA HyperPlus kit (Roche, Cat. 07962410001), following the manufacturer's protocol. The resulting 18 libraries were sequenced in an Illumina NovaSeq 6000 platform using an S4 reagent kit v1.5 (Illumina, Cat. 20028312).

### Example 17.5. Data Processing for Single Nucleotide Variant (SNV) Analysis

Paired-end reads were aligned to human genome GRCh38 with BWA-MEM v0.7.17 (Li, 2013). PCR duplicates were removed with Picard MarkDuplicates v2.19.0 (Broad Institute, 2019). Subsequently, base scores were corrected using GATK BaseRecalibrator and ApplyBQSR v4.1.8.1 (Auwera et al., 2013; DePristo et al., 2011; McKenna et al., 2010). Variants were called using DeepVariant v1.0.0 (Poplin et al., 2018). Relative C to T frequencies were calculated by dividing the total number of C to T variants by the total number of SNVs for each sample. Treatment groups were compared using unpaired t-tests and the statistical significance was determined using the Holm-Sidak method with an alpha of 0.05, without assuming a consistent standard deviation.

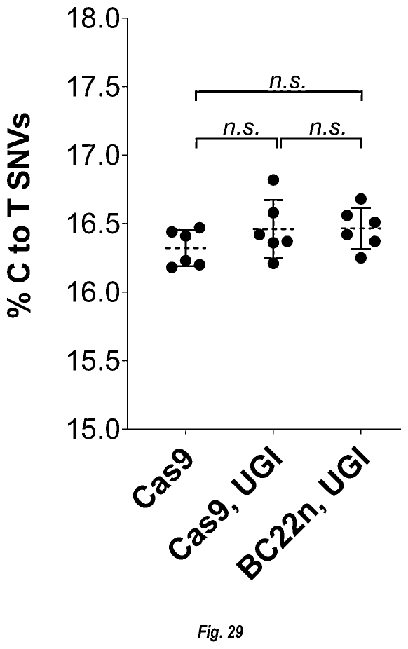

Compared to samples treated with Cas9 mRNA, T cells electroporated with both Cas9 and UGI mRNAs or those treated with BC22n and UGI mRNAs, showed no statistically significant (p<0.05) increase in the frequency of C to T transitions in amplified genomic DNA, demonstrating the absence of detectable homology-independent cytosine deamination events in these samples (Table 45 and FIG. 29).

TABLE 45
Percent C to T transitions of all single nucleotide
variants (SNVs) in amplified genomic DNA
from single T cells harvested 7 days post-electroporation
with different mRNA combinations and
B2M targeting sgRNA G015995
(SD = standard deviation; NA = not applicable).

| | | % C to T SNVs | | | |
|---|---|---|---|---|---|
| | | | | p value | p value |
| sgRNA | mRNA(s) | Mean | SD | (vs. Cas9) | (vs. Cas9, UGI) |
| G015995 | Cas9 | 16.32 | 0.13 | NA | 0.21 |
| G015995 | Cas9, UGI | 16.46 | 0.21 | 0.21 | NA |
| G015995 | BC22n, UGI | 16.47 | 0.15 | 0.11 | 0.96 |

### Example 18. Whole Genome Sequencing of Clonally Expanded eHap1 Cells Following B2M Editing

### Example 18.1. eHap1 Cell Culture

Fully haploid, engineered Hap1 (eHap1) cells were obtained commercially (Horizon Discovery Cat. C669), and cells were cultured in IMDM growth media composed of Iscove's Modified Dulbecco's medium (Thermofisher Cat. 12440053) supplemented with 10% (v/v) of fetal bovine serum (Thermofisher Cat. A3840001) and 1× of Penicillin-Streptomycin (Thermofisher Cat. 15140122). Upon thawing, eHap1 cells were cultured for 48 hours at 37° C. and seeded 24 hours prior to LNP treatments at the density of 6×10\hat{ }5 cells/well in 6-well plates, which were incubated at 37° C. until treatment.

### Example 18.2. eHap1 Editing

The B2M-targeting sgRNA G015991 and mRNAs encoding Cas9 (SEQ ID NO: 11), BC22n (SEQ ID NO: 2), UGI (SEQ ID NO: 26) or BE4MAX (SEQ ID NO: 32) were formulated as individual RNA species in LNPs as described in Example 1. LNPs were applied to T cells in different combinations with at the following concentrations of total RNA: 0.104 μg/mL editor mRNA; 0.55 μg/mL UGI mRNA; 0.4175 μg/mL sgRNA. Different LNP combinations (Table 46) were pre-mixed in IMDM growth media supplemented with 10 μg/mL of recombinant human ApoE3 (Peprotech Cat. 350-02) and incubated at 37° C. for 15 minutes. The culture media of eHap1 cells was removed and each well received 3 mL of LNP mixture. Untreated controls received 3 mL of IMDM growth media supplemented with 10 μg/mL of ApoE3 without LNPs. Cells were incubated for 24 hours at 37° C. and the media was removed and replaced by IMDM growth media.

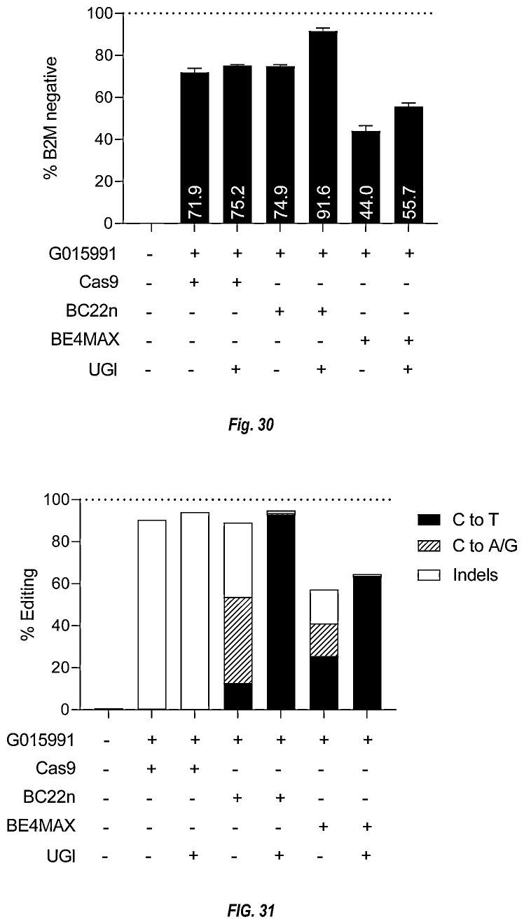

Three and five days after treatment, eHap1 cells were detached, re-seeded at a lower density, and returned to the 37° C. incubator. At the 5-day timepoint, a fraction of cells was stained with anti-B2M antibodies (BioLegend Cat. No. 316304) to evaluate the loss of B2M expression by flow cytometry. These cells were incubated for 30 min at 4° C. with anti-B2M antibodies diluted 1:200 in cell staining buffer (BioLegend® Cat. No. 420201). Cells were subsequently washed, processed on a Cytoflex flow cytometer (Beckman Coulter) and analyzed using the FlowJo software package. eHap1 cells were gated based on size, shape and B2M expression. Table 46 and FIG. 30 show B2M protein expression levels in edited eHap1 cells.

TABLE 46
Mean percent B2M negative eHap1 cells following
editing at B2M with different mRNA combinations.

| | | % B2M KO | |
|---|---|---|---|
| sgRNA | mRNA(s) | Mean | SD |
| None | None | 0.30 | 0.00 |
| G015991 | Cas9 | 71.93 | 1.93 |
| G015991 | Cas9, UGI | 75.20 | 0.46 |
| G015991 | BC22n | 74.87 | 0.67 |
| G015991 | BC22n, UGI | 91.63 | 1.39 |
| G015991 | BE4MAX | 44.00 | 2.54 |
| G015991 | BE4MAX, UGI | 55.70 | 1.66 |

Seven days after treatment, eHap1 cells were detached, the replicates from each treatment group were pooled and the resulting cell suspension was centrifuged. A small fraction of cells from each pool was collected for bulk NGS sequencing as described in Example 1. Table 47 and FIG. 31 show B2M editing levels in these samples. The remaining eHap1 cells were mixed with antibodies against B2M (BioLegend Cat. No. 316304), diluted at 1:200 (v/v) in cell staining buffer (BioLegend® Cat. No. 420201) and incubated for 30 min at 4° C. Cells were subsequently washed and processed in the MA900 fluorescence-activated cell sorting (FACS) instrument (Sony Biotechnology). Single cells negative for B2M expression were individually seeded in 96-well plates. For untreated controls, single cells with positive signal for B2M expression were plated instead. Single cells were incubated for 10 days at 37° C. to allow the establishment and expansion of single-cell clones.

TABLE 47
B2M editing results (NGS sequencing) of eHap1 cells following
treatment with different mRNA combinations and B2M sgRNA
G015991 (Indels = insertions or deletions).

| | | NGS Sequencing | | |
|---|---|---|---|---|
| sgRNA | mRNA(s) | % C to T | % C to A/G | % Indel |
| None | None | 0.19 | 0.24 | 0.27 |
| G015991 | Cas9 | 0.18 | 0.35 | 89.90 |
| G015991 | Cas9, UGI | 0.22 | 0.23 | 93.71 |
| G015991 | BC22n | 12.78 | 40.92 | 35.39 |
| G015991 | BC22n, UGI | 93.08 | 0.55 | 1.24 |
| G015991 | BE4MAX | 25.51 | 15.65 | 16.17 |
| G015991 | BE4MAX, UGI | 63.57 | 0.28 | 0.75 |

### Example 18.3. Clonal Expansion, Extraction of Genomic DNA and Whole Genome Sequencing

After 10 days in culture, clones were visually inspected under an inverted fluorescence microscope. Twelve clones from each treatment group were transferred to 6-well plates to enable further expansion. A small fraction of cells from each clone was collected for NGS sequencing as described in Example 1, while the remaining cells were seeded in 6-plates and cultured at 37° C. until confluence was reached. Based on their on-target editing outcomes, 5 clones per group were selected for whole genome sequencing. Cells from all clonal populations, in addition to one non-clonal sample of eHap1 cells, were lysed and their genomic DNA was extracted using the DNeasy Blood & Tissue kit (Qiagen Cat. 69504) following the manufacturer's protocol. DNA samples were converted into whole genome sequencing libraries using the KAPA HyperPlus kit (Roche, Cat. 07962410001), following the manufacturer's instructions. The resulting 36 libraries were sequenced in an Illumina NovaSeq 6000 platform using an S4 reagent kit v1.5 (Illumina, Cat. 20028312).

### Example 18.4. Data Processing for Single Nucleotide Variant (SNV) Analysis

Reads from each sample were first aligned to the human genome build hg38 with bwa (v0.7.17) (Li, 2013; Li and Durbin, 2010). Alignments were processed with samtools (v1.11) modules fixmate, sort and markdup, consecutively (Kumaran et al., 2019; Li et al., 2009). Variations were called from processed alignments using DeepVariant (v1.0.0) (Poplin et al., 2018). Variants from each sample were then merged using GLnexus (v1.3.1) (Lin et al., 2018; Yun et al., 2021). Variants that occurred in the non-clonal sample of eHap1 cells were excluded from all clonal samples. Variants with read depth below 10 or genotype quality score below 15 were ignored as well. Relative C to T frequencies were calculated by dividing the total number of C to T variants by the total number of SNVs for each sample. Treatment groups were compared using unpaired t-tests and the statistical significance was determined using the Holm-Sidak method with an alpha of 0.05, assuming a consistent standard deviation.

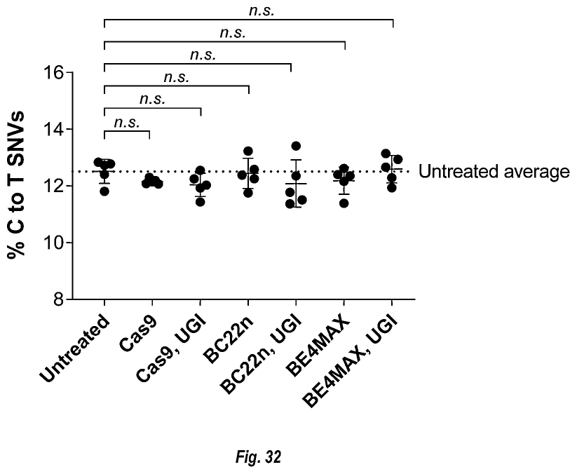

Compared to untreated controls, eHap1 cells treated with Cas9, BC22n or BE4MAX mRNA, in the absence or presence of UGI mRNA, did not show a statistically significant (p<0.05) increase in the frequency of C to T transitions in the genome, demonstrating the absence of detectable homology-independent cytosine deamination events in these samples (Table 48 and FIG. 32).

TABLE 48
Percent C to T transitions among all single nucleotide variants (SNVs)
in clonally expanded eHap1 cells following editing at B2M with
different mRNA combinations (NA = not applicable).

| | | % C to T SNVs | | | |
|---|---|---|---|---|---|
| | | | | p value | Significant? |
| sgRNA | mRNA(s) | Mean | SD | (vs. Group 1) | (p < 0.05?) |
| None | None | 12.52 | 0.43 | NA | NA |
| G015991 | Cas9 | 12.16 | 0.09 | 0.105 | no |
| G015991 | Cas9, UGI | 12.04 | 0.42 | 0.112 | no |
| G015991 | BC22n | 12.44 | 0.53 | 0.820 | no |
| G015991 | BC22n, UGI | 12.08 | 0.84 | 0.336 | no |
| G015991 | BE4MAX | 12.19 | 0.48 | 0.284 | no |
| G015991 | BE4MAX, UGI | 12.59 | 0.48 | 0.789 | no |

### Example 19. Simultaneous Quadruple Edits with BC22n or Cas9 in T Cells after Delivery Via Electroporation or LNP

To assess the amount of structural genomic changes associated with delivery conditions and editing by Cas9 or base editor, T cells treated with electroporation to deliver RNP or lipid nanoparticles (LNP) to deliver four guides and either Cas9 or BC22n were analyzed for cell viability, DNA double-stranded breaks, editing, surface protein expression, and chromosomal structural.

### Example 19.1. T Cell Preparation

Healthy human donor apheresis was obtained commercially (Hemacare), and cells were washed and re-suspended in CliniMACS® PBS/EDTA buffer (Miltenyi Biotec Cat. No. 130-070-525) on the LOVO device. T cells were isolated via negative selection using EasySep™ Human T Cell Isolation Kit (Stemcell Cat. No. 17951). T cells were aliquoted into vials and cryopreserved in a 1:1 formulation of Cryostor® CS10 (StemCell Technologies Cat. No. 07930) and Plasmalyte A (Baxter Cat. No. 2B2522X) for future use.

Upon thaw, T cells were plated at a density of 1.0×10\hat{ }6 cells/mL in OpTmizer-based media containing CTS OpTmizer T Cell Expansion SFM and T Cell Expansion Supplement (ThermoFisher Cat. A1048501), 5% human AB serum (GeminiBio, Cat. 100-512) 1× Penicillin-Streptomycin, 1× Glutamax, 10 mM HEPES, 200 U/mL recombinant human interleukin-2 (Peprotech, Cat. 200-02), 5 ng/ml recombinant human interleukin 7 (Peprotech, Cat. 200-07), and 5 ng/ml recombinant human interleukin 15 (Peprotech, Cat. 200-15). T cells were activated with TransAct™ (1:100 dilution, Miltenyi Biotec) in this media for 72 hours, at which time they were washed and plated in quadruplicate for editing either by electroporation or lipid nanoparticle.

### Example 19.2. Single gRNA and 4 gRNA T Cell Editing with Lipid Nanoparticles

LNPs were formulated generally as in Example 1 with a single RNA species cargo. Cargo was selected from an mRNA encoding BC22n, an mRNA encoding Cas9, an mRNA encoding UGI, sgRNA G015995 targeting B2M, sgRNA G016017 targeting TRAC, sgRNA G016200 targeting TRBC or sgRNA G016086 targeting CIITA. Each LNP was incubated in OpTmizer-based media with cytokines as described above supplemented with 20 ug/ml recombinant human ApoE3 (Peprotech, Cat. 350-02) for 15 minutes at 37° C. Seventy-two hours post activation, T cells were washed and suspended in OpTmizer media with cytokines without human serum. For single sgRNA editing conditions, pre-incubated LNP mix was added to the each well of 100,000 cells to yield a final concentration of 2.3 ug/ml editor mRNA (BC22n or Cas9), 1.1 ug/mL UGI and 4.6 ug/uL G016017. For four-plex sgRNA editing LNP mix was added to the each well of 100,000 cells to yield a final concentration of 2.3 ug/ml editor mRNA (BC22n or Cas9), 1.1 ug/mL UGI, 1.15 ug/uL G015995, 1.15 ug/uL G016017, 1.15 ug/uL G016200 and 1.15 ug/uL G016086. A control group including unedited T cells (no LNP) was also included. At 16 hours post-delivery, a subset of cells was used to measure cell viability and another subset of cells was processed for imaging of γH2AX foci. The remaining T cells continued to expand in culture. Media was changed 5 days and 8 days after activation and on the eleventh day post activation, cells were harvested for analysis by NGS, flow cytometry and UnIT. NGS was performed as in Example 1.

### Example 19.3. Single gRNA and 4 gRNA T Cell Editing with mRNA Electroporation

Electroporation was performed 72 hours post activation. sgRNA G015995 (SEQ ID NO: 182) targeting B2M, sgRNA G016017 (SEQ ID NO: 184) targeting TRAC, sgRNA G016200 (SEQ ID NO:801) targeting TRBC and sgRNA G016086 (SEQ ID NO: 586) were denatured for 2 minutes at 95° C. before cooling at room temperature for 10 minutes. T cells were harvested, centrifuged, and resuspended at a concentration of 12.5×10\hat{ }6 T cells/mL in P3 electroporation buffer (Lonza). For single sgRNA editing conditions, 1×10≡T cells were mixed with 40 ng/uL of editor mRNA (BC22n or Cas9), 10 ng/uL of UGI mRNA and 80 pmols of sgRNA in a final volume of 20 uL of P3 electroporation buffer. For four-plex sgRNA editing conditions, 1×10\hat{ }5 T cells were mixed with 40 ng/uL of editor mRNA (BC22n or Cas9), 10 ng/uL of UGI mRNA and 20 pmols of the four individual sgRNA in a final volume of 20 uL of P3 electroporation buffer. This mix was transferred in quadruplicate to a 96-well Nucleofector™ plate and electroporated using a manufacturer's pulse code. Electroporated T cells were rested in 80 ul of OpTmizer-based media with cytokines before being transferred to a new flat-bottom 96-well plate. A control group including unedited T cells (no EP) was also included. At 16 hours post-delivery, a subset of cells was used to measure cell viability and another subset of cells was processed for imaging of γH2AX foci.

### Example 19.4. Relative Viability Via Cell Titer Glo

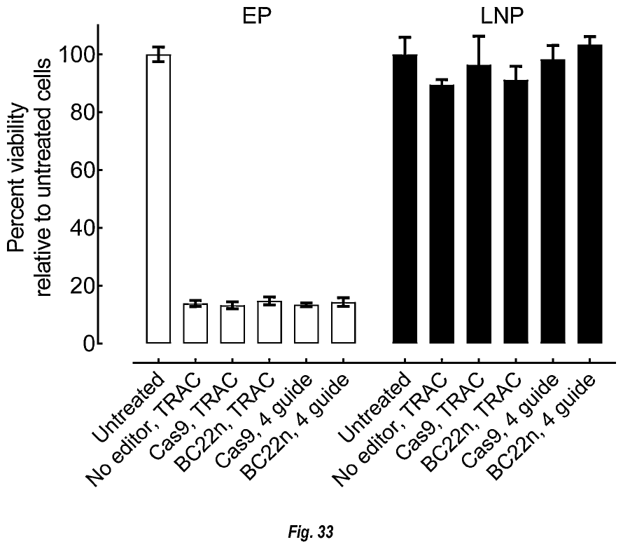

Sixteen hours post electroporation or lipid nanoparticle delivery 20 uL of control or edited cells were removed from original plate and added to a new flat-bottom 96-well plate with black walls (Corning Cat. 3904). CellTiter-Glo® 2.0 (Promega Cat. G9241) was added and samples were processed according to manufacturer's protocol. Relative luminescence units (RLU) were readout by the CLARIstar plus (BMG Labtech) plate reader with gain set at 3600. Relative viability as shown in Table 49 and FIG. 33 was calculated by dividing all sample RLU by the average of untreated control RLU. All electroporation conditions had a greater than 5-fold viability drop from untreated control levels whereas LNP treatment, even with 4 guides editing simultaneously, maintained cell viability at close to untreated control samples.

TABLE 49
Relative cell viability 16 hours following treatment
with various editing and delivery conditions

| | | EP | | LNP | |
|---|---|---|---|---|---|
| Editor | Guide(s) | Mean | SD | Mean | SD |
| No Editor | No guide | 100.00 | 2.52 | 100.00 | 5.90 |
| No Editor | G016017 | 13.88 | 1.05 | 89.48 | 1.77 |
| Cas9 | G016017 | 13.25 | 1.18 | 96.43 | 9.82 |
| BC22n | G016017 | 14.78 | 1.37 | 91.20 | 4.67 |
| Cas9 | 4 guides | 13.45 | 0.65 | 98.30 | 4.80 |
| BC22n | 4 guides | 14.38 | 1.49 | 103.40 | 2.75 |

### Example 19.5. Staining, Imaging and Quantification of γH2AX Foci

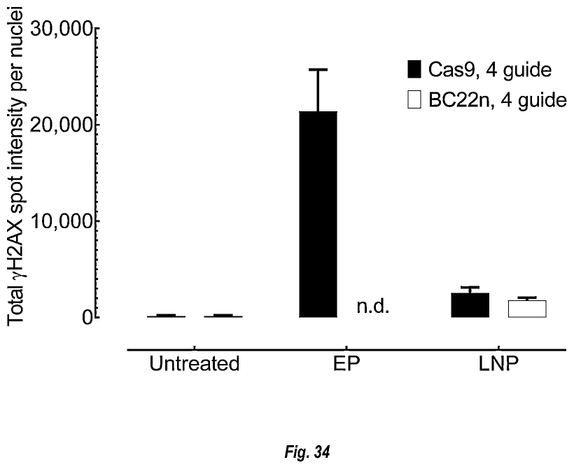

16 hours post electroporation or lipid nanoparticle delivery T cells were cytospun to a slide using Cytospin 4 (Thermo Fisher). After 5 min pre-extraction in PBS/0.5% Trion X-100 on ice, cells were fixed in 4% paraformaldehyde for 10 min. Then, cells were washed in PBS several times and blocked in PBS/0.1% TX-100/1% BSA for 30 min. Primary antibody (Mouse anti-phospho-Histone H2A.X (Ser139) (Millipore Cat. 05-636) was incubated in the blocking buffer at 4° C. overnight. After washed in PBS/0.05% Tween-20 three times, secondary antibody (Goat anti-Mouse IgG Alexa 568 (Thermo Fisher Cat. A31556) was incubated in the blocking buffer at room temperature for 30 min. Cells were washed in PBS/0.05% Tween-20 and nuclei were counter stained with Hoechst 33342. Images were generated by confocal imaging with the Leica SP8. Image analysis was performed via a custom protocol on Thermo Scientific HCS Studio Cell Analysis Software Spot Detector module. Table 50 and FIG. 34 show total γH2AX spot intensity per nuclei following treatment with stated editing and delivery conditions. EP Cas9 with 4 guides samples showed a significant increase in gH2AX foci per nuclei over LNP Cas9-4 guide samples.

TABLE 50
Mean total γH2AX spot intensity per nuclei following treatment with various
editing and delivery conditions

| | No editor | | | Cas9, 4 guides | | | BC22n, 4 guides | | |
|---|---|---|---|---|---|---|---|---|---|
| Delivery | Mean | SD | N | Mean | SD | N | Mean | SD | N |
| Untreated | 134.33 | 95.78 | 5 | | | | | | |
| EP | | | | 21386.62 | 4336.69 | 3 | Not done | | |
| LNP | | | | 2550.88 | 562.77 | 3 | 1770.11 | 291.97 | 5 |

### Example 19.6. Flow Cytometry and NGS Sequencing

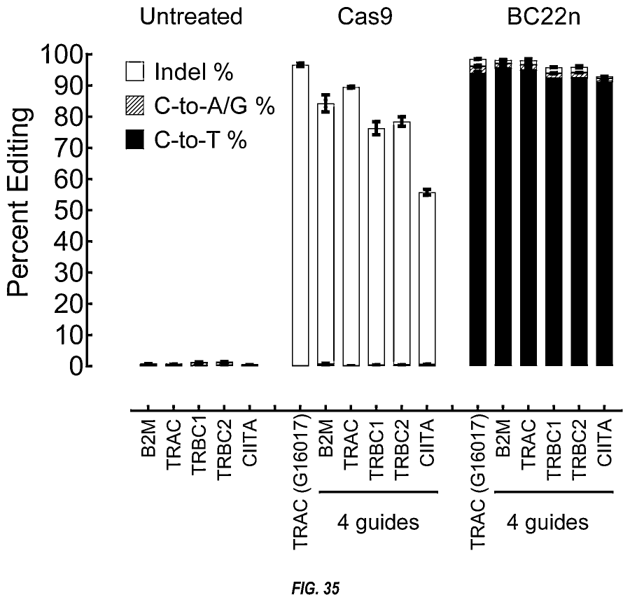

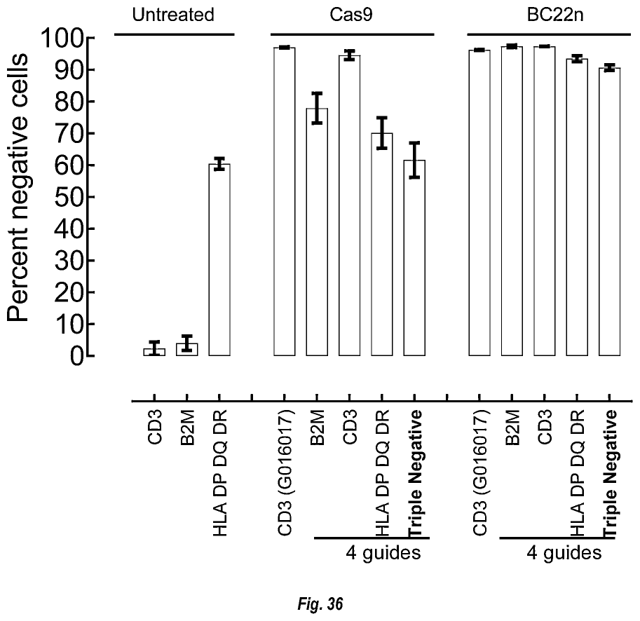

On day 8 post-editing, T cells were phenotyped by flow cytometry to determine B2M, CD3 and HLA II-DR, DP, DQ protein expression as described in Example 6 using antibodies targeting B2M-APC/Fire™ 750 (BioLegend® Cat. No. 316314), CD3-BV605 (BioLegend® Cat. No. 316314) and HLA II-DR, DP, DQ-PE (BioLegend® Cat. No. 361716). DNA samples were subjected to PCR and subsequent NGS analysis, as described in Example 1. Table 51 and FIG. 35 show percent editing at loci of interest following treatment with LNPs. In the condition where 4 guides were delivered by LNP, percent editing is higher at each locus with BC22n than with Cas9. Table 52 and FIG. 36 show surface protein expression of interest following LNP treatment. Editing with BC22n resulted in a greater percentage of triple knockout cells than editing with Cas9.

TABLE 51
Mean percent editing following treatment with stated editing schemes by LNP
delivery.

| | | Readout | C-to-T % | | | C-to-A/G % | | | Indel % | | |
|---|---|---|---|---|---|---|---|---|---|---|---|
| Editor | Guide(s) | Locus | Mean | SD | N | Mean | SD | N | Mean | SD | N |
| No Editor | No guide | B2M | 0.20 | 0.00 | 2 | 0.41 | 0.01 | 2 | 0.13 | 0.01 | 2 |
| | | TRAC | 0.05 | 0.07 | 2 | 0.53 | 0.01 | 2 | 0.09 | 0.01 | 2 |
| | | TRBC1 | 0.25 | 0.07 | 2 | 0.86 | 0.09 | 2 | 0.24 | 0.02 | 2 |
| | | TRBC2 | 0.30 | 0.14 | 2 | 0.85 | 0.04 | 2 | 0.27 | 0.01 | 2 |
| | | CIITA | 0.15 | 0.07 | 2 | 0.26 | 0.03 | 2 | 0.06 | 0.00 | 2 |
| Cas9 | G016017 | TRAC | 0.00 | 0.00 | 2 | 0.05 | 0.00 | 2 | 96.59 | 0.52 | 2 |
| | 4 guides | B2M | 0.30 | 0.14 | 2 | 0.37 | 0.21 | 2 | 83.61 | 2.72 | 2 |
| | | TRAC | 0.00 | 0.00 | 2 | 0.06 | 0.04 | 2 | 89.45 | 0.25 | 2 |
| | | TRBC1 | 0.00 | 0.00 | 2 | 0.35 | 0.01 | 2 | 75.98 | 2.09 | 2 |
| | | TRBC2 | 0.00 | 0.00 | 2 | 0.38 | 0.03 | 2 | 78.07 | 1.51 | 2 |
| | | CIITA | 0.20 | 0.14 | 2 | 0.38 | 0.04 | 2 | 55.23 | 0.92 | 2 |
| BC22n | G016017 | TRAC | 93.90 | 0.57 | 2 | 2.29 | 0.29 | 2 | 2.34 | 0.08 | 2 |
| | 4 guides | B2M | 95.65 | 0.07 | 2 | 1.42 | 0.23 | 2 | 1.07 | 0.16 | 2 |
| | | TRAC | 95.00 | 0.57 | 2 | 1.65 | 0.07 | 2 | 1.44 | 0.43 | 2 |
| | | TRBC1 | 92.40 | 0.00 | 2 | 1.55 | 0.18 | 2 | 1.91 | 0.10 | 2 |
| | | TRBC2 | 92.50 | 0.00 | 2 | 1.62 | 0.02 | 2 | 1.87 | 0.33 | 2 |
| | | CIITA | 91.35 | 0.35 | 2 | 0.93 | 0.04 | 2 | 0.64 | 0.04 | 2 |

TABLE 52
Mean percentage of cells expressing surface following
treatment with stated editing schemes by LNP delivery.

| Editor | Guide | Surface protein | Mean | SD | N |
|---|---|---|---|---|---|
| No | No guide | CD3- | 2.30 | 2.12 | 2 |
| Editor | | B2M- | 4.00 | 2.26 | 2 |
| | | HLA DP DQ DR- | 60.40 | 1.70 | 2 |
| Cas9 | G016017 | CD3- | 97.05 | 0.16 | 2 |
| | 4 guides | B2M- | 77.90 | 4.67 | 2 |
| | | CD3- | 94.54 | 1.34 | 2 |
| | | HLA DP DQ DR- | 70.10 | 4.81 | 2 |
| | | CD3- B2M- HLA DP DQ DR- | 61.58 | 5.42 | 2 |
| BC22n | G016017 | CD3- | 96.20 | 0.13 | 2 |
| | 4 guides | B2M- | 97.35 | 0.37 | 2 |
| | | CD3- | 97.34 | 0.08 | 2 |
| | | HLA DP DQ DR- | 93.46 | 0.89 | 2 |
| | | CD3- B2M-HLA DP DQ DR- | 90.63 | 0.89 | 2 |

### Example 19.7. Measuring Structural Variation and Translocations by UnIT

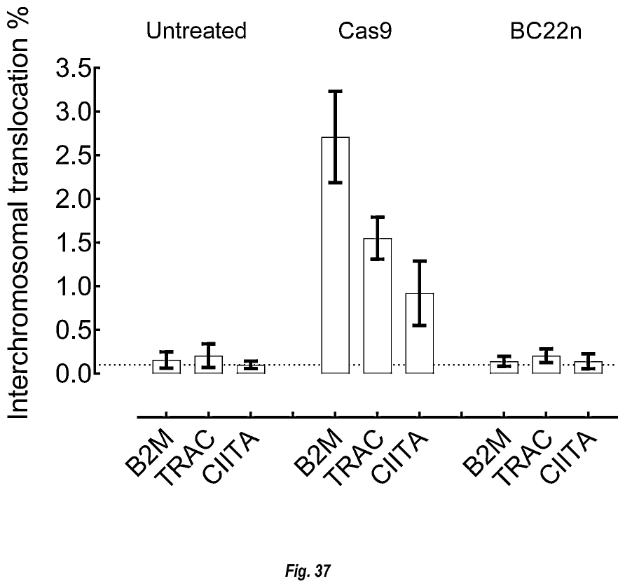

On day 8 post-editing a subset of T cells from the untreated, LNP-Cas9-4 guides and LNP-BC22n-4 guides samples were collected, spun down and resuspended in 100 uL of PBS. gDNA was isolated from the cells using DNeasy Blood & Tissue Kit (Qiagen Cat. 69504). The UnIT structural variant characterization assay was applied to these gDNA samples. High molecular weight genomic DNA is simultaneously fragmented and sequence-tagged (‘tagmented’) with the Tn5 transposase and an adapter with a partial Illumina P5 sequence and a 12 bp unique molecular identifier (UMI). Two sequential PCRs using a primer to P5 and hemi-nested gene specific primers (GSP) imparting the Illumina the P7 sequence to create two Illumina compatible NGS libraries per sample. Sequencing across both directions of the CRISPR/Cas9 targeted cut site with the two libraries allows the inference and quantification of structural variants in DNA repair outcomes after genome editing. If the two fragments were aligned to different chromosomes, the SV was classified as an “inter-chromosomal translocation.” Structural variation results show that interchromosomal translocations are reduced to background levels when multiplex editing is being conducted by BC22n whereas Cas9 multiplex editing leads to significant increases in structural variation, as shown in Table 53 and FIG. 37.

TABLE 53
Mean percent interchromosomal translocations
among total unique molecule identifiers following treatment
with stated editing schemes by LNP delivery.

| Edit | Locus | Mean | SD | N |
|---|---|---|---|---|
| Untreated | B2M | 0.16 | 0.09 | 2 |
| | TRAC | 0.21 | 0.13 | 2 |
| | CIITA | 0.10 | 0.04 | 2 |
| Cas9, 4 guides | B2M | 2.71 | 0.52 | 2 |
| | TRAC | 1.55 | 0.24 | 2 |
| | CIITA | 0.92 | 0.37 | 2 |
| BC22n, 4 guides | B2M | 0.14 | 0.06 | 2 |
| | TRAC | 0.21 | 0.08 | 2 |
| | CIITA | 0.14 | 0.08 | 2 |

### Example 20—CD38 Guide RNA Screening in T Cells with Cas9 and BC22n

### Example 20.1 T Cell Preparation

T cells were edited at the CD38 locus with either Cas9 or with BC22n and UGI mRNAs to assess the editing outcomes and the corresponding loss of CD38 expression.

Healthy human donor apheresis was obtained commercially (Hemacare), and cells were washed and resuspended in CliniMACS PBS/EDTA buffer (Miltenyi Biotec, Cat. No. 130-070-525) on the LOVO device. T cells were isolated via positive selection using CD4 and CD8 magnetic beads (Miltenyi Biotec, Cat. No. 130-030-401/130-030-801) using the CliniMACS Plus and CliniMACS LS disposable kit. T cells were aliquoted into vials and cryopreserved in Cryostor CS10 (StemCell Technologies, Cat. No. 07930) for future use. Upon thaw, T cells were plated at a density of 1.0×10\hat{ }6 cells/mL in T cell X-VIVO 15 expansion media composed of X-VIVO 15 (Lonza, Cat. No. BE02-06Q) containing 5% (v/v) of fetal bovine serum (ThermoFisher, Cat. No. A3160902), 50 μM (1×) 2-Mercaptothanol (ThermoFisher, Cat. No. 31350010), 1% of Penicillin-Streptomycin (ThermoFisher, Cat. No. 15140122), 1 M N-acetyl L-cystine (Fisher, Cat. No. ICN19460325) diluted in phosphate buffered saline (PBS) and normalized to pH 7, supplemented with 100 U/mL of recombinant human interleukin-2 (Peprotech, Cat. No. 200-02), 5 ng/mL recombinant human interleukin-7 (Peprotech, Cat. No. 200-07) and 5 ng/mL recombinant human interleukin-15 (Peprotech, Cat. No. 200-15). T cells were activated with TransAct™ (1:100 dilution, Miltenyi Biotec, Cat. No. 130-111-160). Cells were expanded for 72 hours at 37° C. prior to mRNA electroporation.

### Example 20.2 T Cell Editing with RNA Electroporation

Solutions containing mRNA encoding Cas9 protein, BC22n, or UGI were prepared in sterile water. 50 μM CD38 targeting sgRNAs were removed from their storage plates and denatured for 2 minutes at 95° C. and incubated at room temperature for 5 minutes. Seventy-two hours post activation, T cells were harvested, centrifuged, and resuspended at a concentration of 12.5×10\hat{ }6 T cells/mL in P3 electroporation buffer (Lonza). Messenger RNAs were prepared as described in Example 1. For each well to be electroporated, 1×10\hat{ }5 T cells were mixed with 200 ng of Cas9 or BC22n mRNAs, 200 ng of UGI mRNA and 20 pmols of sgRNA as described in Table 54 in a final volume of 20 μL of P3 electroporation buffer. This mix was transferred in duplicate to a 96-well Nucleofector™ plate and electroporated using the manufacturer's pulse code. Electroporated T cells were immediately rested in 80 μL of X-VIVO 15 media without cytokines for 15 minutes before being transferred to new flat-bottom 96-well plates containing an additional 90 μL of X-VIVO 15 media supplemented with 2× cytokines. The resulting plates were incubated at 37° C. for 10 days. To promote expansion, T cells were split at the ratios of 1:4 and 1:3 on days 3 and 6 post-electroporation, respectively, using fresh X-VIVO 15 media with 1× cytokines. On day 9 post-electroporation, cells were split 1:2 in 2 U-bottom plates and one plate was collected for NGS sequencing, while the other plate was used for flow cytometry on Day 10.

### Example 20.3 Flow Cytometry and NGS Sequencing

On day 10 post-editing, T cells were phenotyped by flow cytometry to determine CD38 receptor expression. Briefly, T cells were incubated for 30 min at 4° C. with a mixture of antibodies against CD3 (BioLegend, Cat. No. 317340), CD4 (BioLegend, Cat. No. 300537), CD8 (BioLegend, Cat. No. 344706) diluted at 1:200 and CD38 (BioLegend, Cat. No. 303546), diluted at 1:100 in cell staining buffer (BioLegend, Cat. No. 420201). Cells were subsequently washed and stained with viability antibody DAPI (BioLegend, Cat. No. 422801) diluted at 1:10,000 in cell staining buffer. Cells were then processed on a Cytoflex flow cytometer (Beckman Coulter) and analyzed using the FlowJo software package. T cells were gated based on size, shape, viability, and CD38 expression.

On day 9 DNA samples were subjected to PCR and subsequent NGS analysis, as described in Example 1. Table 54 shows CD38 gene editing and CD38 positive results for cells edited with BC22n or Cas9.

TABLE 54
Percent editing and percent of CD38 positive cells following CD38 editing
with Cas9 or BC22n base editor

| | Cas9 % Indels | | | Cas9 % CD38+ | | | BC22n % C to T | | | BC22 % CD38+ | | |
|---|---|---|---|---|---|---|---|---|---|---|---|---|
| Guide ID | Mean | SD | N | Mean | SD | N | Mean | SD | N | Mean | SD | N |
| G019761 | 94.82 | 0.97 | 2 | 22.35 | 0.49 | 2 | 87.21 | 1.57 | 2 | 56.45 | 2.05 | 2 |
| G019762 | 98.19 | 0.63 | 2 | 15.45 | 1.06 | 2 | 85.04 | 0.42 | 2 | 58.55 | 0.07 | 2 |
| G019763 | 91.42 | 1.77 | 2 | 8.17 | 2.38 | 2 | 87.01 | 0.51 | 2 | 2.53 | 0.21 | 2 |
| G019764 | 87.78 | 1.04 | 2 | 11.80 | 0.71 | 2 | 87.00 | 2.33 | 2 | 41.40 | 2.12 | 2 |
| G019765 | 96.43 | 0.40 | 2 | 13.15 | 2.33 | 2 | 81.51 | 5.02 | 2 | 60.40 | 1.27 | 2 |
| G019766 | 97.61 | 0.06 | 2 | 5.31 | 1.15 | 2 | 80.51 | 2.67 | 2 | 61.80 | 0.85 | 2 |
| G019767 | 97.54 | 0.08 | 2 | 17.45 | 0.21 | 2 | 81.92 | 0.46 | 2 | 62.55 | 3.46 | 2 |
| G019768 | 96.65 | 0.96 | 2 | 2.19 | 1.06 | 2 | 89.45 | 0.78 | 2 | 2.49 | 1.18 | 2 |
| G019769 | 95.63 | 2.44 | 2 | 2.58 | 0.35 | 2 | 69.93 | 5.38 | 2 | 14.40 | 4.81 | 2 |
| G019770 | 97.74 | 1.26 | 2 | 1.15 | 0.59 | 2 | 85.06 | 5.81 | 2 | 38.80 | 3.54 | 2 |
| G019771 | 96.90 | 0.54 | 2 | 1.18 | 0.18 | 2 | 87.34 | 0.16 | 2 | 1.39 | 0.26 | 2 |
| G019772 | 96.38 | 0.05 | 2 | 14.50 | 1.98 | 2 | 88.86 | 0.71 | 2 | 70.25 | 1.63 | 2 |
| G019773 | 95.38 | 2.21 | 2 | 6.70 | 0.57 | 2 | 79.83 | 0.15 | 2 | 64.15 | 4.45 | 2 |
| G019774 | 97.12 | 0.07 | 2 | 14.55 | 0.92 | 2 | 71.14 | 2.52 | 2 | 68.60 | 2.83 | 2 |
| G019775 | 95.33 | 3.04 | 2 | 16.75 | 1.63 | 2 | 92.02 | 0.11 | 2 | 74.90 | 0.28 | 2 |
| G019776 | 98.87 | 0.45 | 2 | 1.95 | 1.04 | 2 | 84.25 | 0.57 | 2 | 11.45 | 1.63 | 2 |
| G019777 | 97.87 | 0.54 | 2 | 14.00 | 1.27 | 2 | 86.76 | 1.32 | 2 | 71.75 | 2.62 | 2 |
| G019778 | 95.81 | 1.24 | 2 | 13.75 | 1.48 | 2 | 86.96 | 1.81 | 2 | 62.30 | 2.26 | 2 |
| G019779 | 97.78 | 0.26 | 2 | 10.87 | 2.17 | 2 | 88.77 | 0.53 | 2 | 74.35 | 0.07 | 2 |
| G019780 | 95.75 | 1.84 | 2 | 14.00 | 2.55 | 2 | 89.81 | 0.30 | 2 | 69.05 | 3.18 | 2 |
| G019781 | 93.81 | 2.35 | 2 | 15.90 | 0.85 | 2 | 86.84 | 4.62 | 2 | 68.30 | 0.71 | 2 |
| G019782 | 93.10 | 4.65 | 2 | 10.21 | 2.82 | 2 | 82.19 | 1.92 | 2 | 63.15 | 0.49 | 2 |
| G019783 | 97.88 | 0.30 | 2 | 2.58 | 0.40 | 2 | 77.26 | 4.30 | 2 | 64.10 | 0.14 | 2 |
| G019784 | 97.61 | 0.86 | 2 | 16.80 | 2.69 | 2 | 76.82 | 0.81 | 2 | 69.00 | 0.71 | 2 |
| G019785 | 98.30 | 0.27 | 2 | 1.58 | 0.66 | 2 | 80.95 | 1.14 | 2 | 33.90 | 5.80 | 2 |
| G019786 | 86.22 | 3.34 | 2 | 28.10 | 4.67 | 2 | 88.42 | 2.78 | 2 | 72.35 | 5.02 | 2 |
| G019787 | 98.76 | 0.45 | 2 | 2.06 | 0.86 | 2 | 80.23 | 2.14 | 2 | 1.89 | 0.83 | 2 |
| G019788 | 96.34 | 1.98 | 2 | 11.05 | 0.92 | 2 | 90.88 | 0.06 | 2 | 1.88 | 0.21 | 2 |
| G019789 | 97.68 | 1.33 | 2 | 15.80 | 1.27 | 2 | 67.12 | n.a. | 1 | 52.85 | 1.34 | 2 |
| G019790 | 94.58 | 1.87 | 2 | 12.15 | 0.07 | 2 | 89.81 | 0.22 | 2 | 77.10 | 2.69 | 2 |
| G019791 | 97.79 | 0.90 | 2 | 3.25 | 0.86 | 2 | 79.56 | 3.97 | 2 | 9.51 | 0.62 | 2 |
| G019792 | 96.99 | 0.13 | 2 | 9.19 | 0.11 | 2 | 85.20 | 4.14 | 2 | 64.65 | 0.35 | 2 |
| G019793 | 96.13 | 0.29 | 2 | 19.95 | 2.33 | 2 | 85.55 | 2.40 | 2 | 71.40 | 2.69 | 2 |
| G019794 | 98.30 | 0.28 | 2 | 12.75 | 0.07 | 2 | 76.28 | 0.67 | 2 | 31.40 | 0.85 | 2 |
| G019795 | 97.56 | 0.59 | 2 | 2.52 | 0.56 | 2 | 91.40 | 2.37 | 2 | 1.22 | 0.34 | 2 |
| G019796 | 97.70 | 1.27 | 2 | 7.60 | 2.87 | 2 | 92.29 | 0.28 | 2 | 6.52 | 0.16 | 2 |
| G019797 | 97.04 | 0.26 | 2 | 9.80 | 2.26 | 2 | 87.45 | 0.46 | 2 | 3.07 | 0.84 | 2 |
| G019798 | 97.56 | 1.17 | 2 | 4.92 | 0.93 | 2 | 89.68 | 0.89 | 2 | 59.05 | 1.34 | 2 |
| G019799 | 97.65 | 0.22 | 2 | 8.83 | 1.46 | 2 | 87.59 | 0.90 | 2 | 66.85 | 0.49 | 2 |
| G019800 | 76.71 | 3.24 | 2 | 25.45 | 4.88 | 2 | 74.46 | 2.84 | 2 | 16.00 | 3.54 | 2 |
| G019801 | 92.76 | 3.74 | 2 | 6.10 | 2.28 | 2 | 82.69 | 0.52 | 2 | 33.65 | 4.03 | 2 |
| G019802 | 64.21 | 7.62 | 2 | 33.70 | 5.52 | 2 | 69.14 | 0.45 | 2 | 73.00 | 0.42 | 2 |
| G019803 | 50.46 | 6.67 | 2 | 43.50 | 5.23 | 2 | 45.01 | 1.82 | 2 | 55.40 | 1.98 | 2 |
| G019804 | 89.70 | 3.71 | 2 | 7.05 | 3.23 | 2 | 21.88 | 0.42 | 2 | 67.25 | 2.76 | 2 |
| G019805 | 52.39 | 2.21 | 2 | 40.30 | 0.14 | 2 | 78.69 | 4.52 | 2 | 9.15 | 1.77 | 2 |
| G019806 | 93.47 | 0.40 | 2 | 4.64 | 0.82 | 2 | 72.68 | 1.54 | 2 | 21.30 | 4.95 | 2 |
| G019807 | 86.45 | 2.17 | 2 | 12.70 | 1.84 | 2 | 63.32 | 3.90 | 2 | 65.85 | 0.21 | 2 |
| G019808 | 97.15 | 0.70 | 2 | 2.23 | 1.37 | 2 | 32.87 | 6.70 | 2 | 64.10 | 1.84 | 2 |
| G019809 | 41.04 | 6.38 | 2 | 56.35 | 4.31 | 2 | 68.30 | 3.73 | 2 | 27.50 | 5.94 | 2 |
| G019810 | 46.14 | 4.29 | 2 | 52.05 | 5.16 | 2 | 78.63 | 2.98 | 2 | 10.40 | 2.97 | 2 |
| G019811 | 95.15 | 0.67 | 2 | 14.05 | 1.06 | 2 | 83.21 | 2.11 | 2 | 4.42 | 2.68 | 2 |
| G019812 | 93.95 | 1.06 | 2 | 4.46 | 2.11 | 2 | 84.23 | 5.13 | 2 | 24.90 | 2.55 | 2 |
| G019813 | 96.79 | 1.07 | 2 | 2.51 | 0.60 | 2 | 75.68 | 3.13 | 2 | 5.51 | 1.97 | 2 |
| G019814 | 87.26 | 0.64 | 2 | 11.56 | 2.75 | 2 | 72.31 | 1.65 | 2 | 13.80 | 2.83 | 2 |
| G019815 | 77.53 | 7.29 | 2 | 18.65 | 3.89 | 2 | 86.69 | 0.87 | 2 | 8.47 | 0.74 | 2 |
| G019816 | 76.89 | 1.37 | 2 | 38.25 | 6.86 | 2 | 87.17 | 2.00 | 2 | 8.82 | 2.94 | 2 |
| G019817 | 90.53 | 1.09 | 2 | 5.95 | 1.39 | 2 | 72.59 | 1.92 | 2 | 33.85 | 1.34 | 2 |
| G019818 | 97.71 | 0.21 | 2 | 1.69 | 0.57 | 2 | 77.99 | 3.46 | 2 | 14.15 | 3.75 | 2 |
| G019819 | 94.33 | 1.87 | 2 | 3.70 | 2.29 | 2 | 84.14 | 3.95 | 2 | 2.47 | 0.45 | 2 |
| G019820 | 82.74 | 4.09 | 2 | 21.00 | 3.39 | 2 | 81.28 | 0.66 | 2 | 14.80 | 2.12 | 2 |
| G019821 | 70.98 | 8.64 | 2 | 26.80 | 8.49 | 2 | 73.39 | 3.46 | 2 | 11.09 | 3.27 | 2 |
| G019822 | 46.59 | 2.28 | 2 | 50.80 | 5.09 | 2 | 58.43 | 2.69 | 2 | 73.55 | 0.21 | 2 |
| G019823 | 93.48 | 2.48 | 2 | 4.04 | 1.94 | 2 | 79.65 | 0.80 | 2 | 24.45 | 6.01 | 2 |
| G019824 | 95.90 | 0.76 | 2 | 6.32 | 1.92 | 2 | 89.11 | 1.03 | 2 | 74.75 | 3.18 | 2 |
| G019825 | 29.68 | 7.06 | 2 | 56.10 | 0.57 | 2 | 39.49 | 6.14 | 2 | 66.20 | 3.54 | 2 |
| G019826 | 92.62 | 1.03 | 2 | 3.74 | 1.05 | 2 | 69.37 | 5.37 | 2 | 61.70 | 0.42 | 2 |
| G019827 | 39.32 | 4.26 | 2 | 54.80 | 2.55 | 2 | 48.52 | 0.86 | 2 | 38.15 | 1.20 | 2 |
| G019828 | 87.90 | 3.26 | 2 | 9.98 | 1.16 | 2 | 80.25 | 0.57 | 2 | 7.40 | 2.33 | 2 |
| G019829 | 31.95 | 2.81 | 2 | 63.40 | 1.98 | 2 | 76.10 | 2.57 | 2 | 14.50 | 2.69 | 2 |
| G019830 | 81.01 | 6.21 | 2 | 16.45 | 3.32 | 2 | 83.57 | 1.78 | 2 | 4.29 | 1.12 | 2 |
| G019831 | 98.29 | 0.30 | 2 | 0.91 | 0.25 | 2 | 71.43 | 0.65 | 2 | 2.05 | 0.84 | 2 |
| G019832 | 62.85 | 1.73 | 2 | 34.70 | 3.54 | 2 | 90.87 | 2.45 | 2 | 71.05 | 6.01 | 2 |
| G019833 | 92.66 | 0.94 | 2 | 4.28 | 0.82 | 2 | 75.96 | 0.45 | 2 | 18.40 | 2.26 | 2 |
| G019834 | 94.43 | 0.37 | 2 | 2.75 | 0.76 | 2 | 76.23 | 0.91 | 2 | 69.00 | 2.83 | 2 |
| G019835 | 42.83 | n.a. | 1 | 49.95 | 5.73 | 2 | 51.27 | 8.38 | 2 | 43.95 | 6.72 | 2 |
| G019836 | No data | | | 46.05 | 5.02 | 2 | 31.44 | 6.72 | 2 | 75.10 | 0.00 | 2 |
| G019837 | 43.86 | 6.82 | 2 | 49.85 | 5.44 | 2 | 63.03 | 1.17 | 2 | 51.50 | 1.70 | 2 |
| G019838 | 75.85 | 0.40 | 2 | 24.25 | 0.64 | 2 | 74.87 | 1.14 | 2 | 19.05 | 2.62 | 2 |
| G019839 | 97.44 | n.a.. | 1 | 1.82 | 0.21 | 2 | 69.11 | 4.60 | 2 | 40.55 | 2.05 | 2 |
| G019840 | 59.58 | 4.30 | 2 | 37.90 | 0.85 | 2 | 47.06 | 4.04 | 2 | 73.05 | 1.06 | 2 |
| G019841 | 96.14 | n.a. | 1 | 3.60 | 1.68 | 2 | 69.28 | 2.55 | 2 | 27.15 | 2.33 | 2 |
| G019842 | 33.72 | 6.77 | 2 | 58.65 | 2.05 | 2 | 29.32 | 2.31 | 2 | 71.50 | 3.82 | 2 |
| G019843 | 6.02 | 0.04 | 2 | 73.15 | 0.49 | 2 | 20.85 | 1.36 | 2 | 70.75 | 0.35 | 2 |
| G019844 | 85.26 | 12.45 | 2 | 6.30 | 0.47 | 2 | 76.89 | 3.63 | 2 | 39.45 | 3.75 | 2 |
| G019845 | 36.76 | 2.44 | 2 | 55.25 | 3.18 | 2 | 62.90 | 1.02 | 2 | 60.15 | 2.62 | 2 |
| G019846 | 25.85 | 4.33 | 2 | 64.80 | 2.69 | 2 | 57.55 | 1.80 | 2 | 63.95 | 4.88 | 2 |
| G019847 | 58.00 | 5.06 | 2 | 37.85 | 2.19 | 2 | 71.58 | 0.74 | 2 | 42.55 | 5.16 | 2 |
| G019848 | 47.39 | 6.18 | 2 | 46.90 | 5.09 | 2 | 71.78 | 4.09 | 2 | 67.15 | 3.32 | 2 |

No data means sample had technical fail in all replicates.
N.A. is used when only one replicate was available and thus it was not applicable to calculate a standard deviation.

### Example 21. Base Editing with Nme2Cas9 Base Editor and Chemically Modified sgRNA in HepG2 Cells

Base editor constructs comprising an APOBEC3A deaminase domain fused to Nme2Cas9 D16A nickase were tested for base conversion efficiency with various guide designs in HepG2 cells.

HepG2 cells constitutively overexpress solute carrier family 10 member 1 (SLC10A1) (HepG2-NTCP, Seeger et al. Mol Ther Nucleic Acids. 2014 Dec.; 3(12): e216) were thawed and resuspended in Dulbecco's Modified Eagle's Medium (DMEM) supplemented with 10% Fetal Bovine Serum (FBS) (Media Y) followed by centrifugation. The supernatant was discarded and the cells were resuspended in Media Y and plated at a density of 25,000 cells per well in a 96-well collagen coated plate (Corning, Cat. 354407) in 100 uL of Media Y.

Nme2Cas9 base editor mRNAs were prepared by in vitro transcription essentially as described in Example 1 from plasmids encoding SEQ ID No: 304 (2XNLS N-terminal, lxC-terminal NLS Nme2Cas9 base editor), SEQ ID No: 310 (2XNLS N-terminal, NLS Nme2Cas9 base editor), and SEQ ID No: 301 (1×C-term NLS Nme2Cas9 base editor). SpyCas9 mRNA and uracil glycosylase inhibitor (UGI) mRNA (SEQ ID No: 34) were transcribed from plasmids using the same method.

Chemically modified NmeCas9 sgRNAs targeted to NTCP, with different PAM sequences, (G020927, G020928) or VEGF (G020073) and SpyCas9 sgRNA targeted to NTCP (G020929) were synthesized using routine methods.

The tested NmeCas9 sgRNAs targeting NTCP include a 24 nucleotide guide sequence (as represented by N) and a guide scaffold as follows: mN*mNNNNNNNNmNNNmNNNNNNNNNNNNmGUUGmUmAmGmCUCCCmUmGm AmAmAmCmCGUUmGmCUAmCAAU*AAGmGmCCmGmUmCmGmAmAmAmGmAm UGUGCmCGCmAmAmCmGCUCUmGmCCmUmUmCmUGmGCmAmUC*mG*mU*mU (SEQ ID NO: 522), where A, C, G, U, and N are adenine, cytosine, guanine, uracil, and any ribonucleotide, respectively, unless otherwise indicated. An m is indicative of a 2′O-methyl modification, and an * is indicative of a phosphorothioate linkage between the nucleotides. Unmodified and modified versions of the guides are provided in Table 5C. Guide RNA, editor mRNA, and UGI mRNA were mixed at a 1:1:1 weight ratio with premixed transfection reagent containing Lipid A, cholesterol, DSPC, and PEG2k-DMG in a 50:38:9:3 molar ratio, respectively. Reagents were combined at a lipid amine to RNA phosphate (N:P) molar ratio of about 6.0. RNA-lipid mixture was mixed approximately 1:1 with 10% FBS media and incubated for 10 minutes. Post-incubation, the cells were treated with the RNA-lipid mixture in an 8-point, 2-fold serial dilution starting at 400 ng total editor RNA per well.

At 72 hours post-treatment, cells were lysed for NGS analysis as provided in Example 1.

Dose response of editing efficiency to guide concentration was performed in triplicate. Table 55 shows mean editing percentages calculated [at each guide concentration and a calculated EC50 value. The target site in VEGFA is prone to indel formation due to high GC content. All editor miRNAs achieved the same maximum C to T editing. There were slight differences in EC50 where SEQ ID No: 310 mRNA outperformed SEQ ID No: 304 and SEQ ID No: 301.

TABLE 55
Mean editing percentages in HepG2-NTCP cells at VEGFA locus (G020073)
and Nme2Cas9 base editor

| | Nme-base | C-to-T % | | | | C-to-A/G % | | | Indel % | | |
|---|---|---|---|---|---|---|---|---|---|---|---|
| mRNA | editor (ng) | Mean | SD | n | EC50 | Mean | SD | n | Mean | SD | n |
| SEQ ID No: | 0 | 0.20 | 0.14 | 2 | 6.86 | 0.80 | 0.00 | 2 | 0.70 | 0.14 | 2 |
| 304 | 6.25 | 36.05 | 1.63 | 2 | | 2.30 | 0.28 | 2 | 14.25 | 0.21 | 2 |
| | 12.5 | 49.70 | 1.13 | 2 | | 2.95 | 0.49 | 2 | 16.35 | 1.06 | 2 |
| | 25 | 57.60 | 0.42 | 2 | | 2.75 | 0.07 | 2 | 17.45 | 0.21 | 2 |
| | 50 | 67.15 | 1.34 | 2 | | 2.65 | 0.07 | 2 | 17.90 | 0.14 | 2 |
| | 100 | 70.50 | 0.99 | 2 | | 2.65 | 0.07 | 2 | 18.80 | 0.28 | 2 |
| | 200 | 72.75 | 0.49 | 2 | | 2.90 | 0.57 | 2 | 18.75 | 0.49 | 2 |
| | 400 | 72.00 | 0.14 | 2 | | 3.10 | 0.14 | 2 | 20.40 | 0.99 | 2 |
| SEQ ID No: | 0 | 0.50 | 0.00 | 1 | 3.86 | 1.00 | 0.00 | 1 | 0.60 | 0.00 | 1 |
| 310 | 6.25 | 49.60 | 3.25 | 2 | | 2.10 | 0.14 | 2 | 14.05 | 0.21 | 2 |
| | 12.5 | 57.05 | 0.78 | 2 | | 2.50 | 0.42 | 2 | 16.50 | 0.14 | 2 |
| | 25 | 66.95 | 0.49 | 2 | | 2.40 | 0.00 | 2 | 16.25 | 0.21 | 2 |
| | 50 | 71.45 | 0.35 | 2 | | 2.40 | 0.00 | 2 | 17.25 | 0.07 | 2 |
| | 100 | 71.60 | 0.85 | 2 | | 2.55 | 0.35 | 2 | 17.90 | 0.14 | 2 |
| | 200 | 73.70 | 0.28 | 2 | | 2.65 | 0.07 | 2 | 19.00 | 0.28 | 2 |
| | 400 | 73.80 | 0.14 | 2 | | 3.05 | 0.35 | 2 | 18.70 | 0.42 | 2 |
| SEQ ID No: | 0 | 0.55 | 0.49 | 2 | 4.84 | 1.05 | 0.21 | 2 | 0.55 | 0.07 | 2 |
| 301 | 6.25 | 45.55 | 0.21 | 2 | | 2.60 | 0.28 | 2 | 17.15 | 0.64 | 2 |
| | 12.5 | 56.40 | 1.84 | 2 | | 2.80 | 0.00 | 2 | 19.40 | 0.14 | 2 |
| | 25 | 64.95 | 1.06 | 2 | | 3.15 | 0.35 | 2 | 19.40 | 1.70 | 2 |
| | 50 | 70.70 | 0.28 | 2 | | 3.25 | 0.21 | 2 | 20.00 | 0.71 | 2 |
| | 100 | 72.20 | 2.83 | 2 | | 2.75 | 0.21 | 2 | 19.80 | 1.56 | 2 |
| | 200 | 70.90 | 0.99 | 2 | | 3.15 | 0.35 | 2 | 19.70 | 0.85 | 2 |
| | 400 | 71.80 | 0.14 | 2 | | 3.45 | 0.21 | 2 | 20.95 | 0.35 | 2 |

### Example 22. Base Editing with Chemically Modified sgRNA in PMH

Base editor constructs comprising an APOBEC3A deaminase domain fused to Nme2Cas9 nickase were tested for base conversion efficiency with various guide designs in primary mouse hepatocytes (PMH).

PMH (In Vitro ADMET Laboratories, cat #MC148) were thawed and resuspended in 50 mL Cryopreserved Hepatocyte Recovery Media (CHRM) (Invitrogen, CM7000) followed by centrifugation. Cells were resuspended in hepatocyte medium with plating supplements: Williams' E Medium Plating Supplements with FBS content (Gibco, Cat. A13450). Cells were pelleted by centrifugation, resuspended in media and plated at a density of 20,000 cells/well on Bio-coat collagen I coated 96-well plates (Corning #354407). Plated cells were allowed to settle and adhere for 4-6 hours in a tissue culture incubator at 37° C. and 5% CO2 atmosphere. After incubation cells were checked for monolayer formation and were washed once and plated with 100 uL hepatocyte maintenance medium: Williams' E Medium (Gibco, Cat. A12176-01) plus supplement pack (Gibco, Cat. CM3000).

Nme2Cas9 base editor mRNAs, SEQ ID No: 304, SEQ ID No: 310, and SEQ ID No: 301; and uracil glycosylase inhibitor (UGI) mRNA (SEQ ID No: 34) were prepared as described in Example 1 and paired with a series of chemically modified sgRNA targeted to mouse TTR and screened at a single dose of 128 ng of base editor mRNA. At 72 hours post-treatment, cells were lysed for NGS analysis as provided in Example 1. The mean editing of representative guides (ratio of edit types) are shown in Table 56.

TABLE 56
Mean editing percentages in PMH cells using modified gRNAs targeting the
TTR locus and an Nme2Cas9 base editor. “n/a” means SD is not applicable
when only 1 replicate is reported.

| | | C-to-T % | | | C-to-A/G % | | | Indel % | | |
|---|---|---|---|---|---|---|---|---|---|---|
| mRNA | Guide | Mean | SD | N | Mean | SD | N | Mean | SD | N |
| SEQ ID No: | G021237 | 69.00 | 5.94 | 2 | 3.65 | 1.63 | 2 | 9.00 | 3.39 | 2 |
| 304 | G021249 | 47.15 | 2.33 | 2 | 1.20 | 0.14 | 2 | 1.45 | 1.34 | 2 |
| | G021321 | 7.25 | 0.92 | 2 | 0.25 | 0.21 | 2 | 91.25 | 0.92 | 2 |
| SEQ ID No: | G021237 | 76.75 | 1.34 | 2 | 4.35 | 1.48 | 2 | 7.95 | 1.34 | 2 |
| 310 | G021249 | 54.05 | 4.17 | 2 | 1.20 | 0.28 | 2 | 1.60 | 0.42 | 2 |
| | G021321 | 0.50 | n/a | 1 | 0.10 | n/a | 1 | 99.00 | n/a | 1 |
| SEQ ID No: | G021237 | 73.55 | 5.44 | 2 | 5.15 | 2.47 | 2 | 12.50 | 1.70 | 2 |
| 301 | G021249 | 53.30 | 5.52 | 2 | 1.05 | 0.21 | 2 | 2.15 | 1.63 | 2 |
| | G021321 | 7.05 | 1.20 | 2 | 0.55 | 0.21 | 2 | 90.40 | 2.97 | 2 |

### Example 23. In Vivo Base Editing with gRNA

The editing efficiency of the modified gRNAs with different mRNAs were tested with a Nme2Cas9 base editor construct in the mouse model.

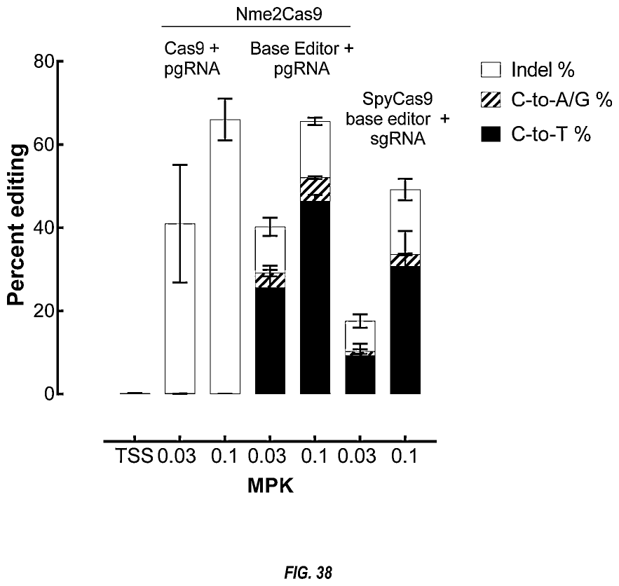

LNPs were generally prepared as described in Example 1 with a single RNA species as cargo. The LNPs used were prepared with Lipid A, cholesterol, DSPC, and PEG2k-DMG in a 50:38:9:3 molar ratio, respectively. The LNPs were formulated with a lipid amine to RNA phosphate (N:P) molar ratio of about 6. The LNPs were formulated as described in Example 1, except that each component, guide RNA, or mRNA was formulated individually into an LNP, and the LNP were mixed prior to administration as described in Table 57. For Nme2Cas9 and Nme2Cas9 base editor samples, LNPs were mixed at a ratio of 2:1 by weight of gRNA to editor mRNA cargo. For SpyCas9 base editor samples, LNPs were mixed at a ratio of 1:2 by weight of gRNA to editor mRNA cargo. Dose, as indicated in Table 57 and FIG. 38, is calculated based on the combined RNA weight of gRNA and editor mRNA. Base editor samples were treated with an additional 0.03 mpk of UGI mRNA. Transport and storage solution (TSS) used in LNP preparation was dosed in the experiment as a vehicle-only negative control.

The tested NmeCas9 gRNA (G021844) including linkers has the following modification pattern of (N)₂₄

(SEQ ID NO: 519)
GUUGmUmAmGmCUCCCmUmUmC(L1)mGmAmCmCGUUmGmCUAmCAAU*
AAGmGmCCmGmUmC(L1)mGmAmUGUGCmCGmCAAmCGCUCUmGmCC
(L1)GGCAUCG*mU*mU,

where A, C, G, U, and N are adenine, cytosine, guanine, uracil, and any ribonucleotide, respectively, unless otherwise indicated. An m is indicative of a 2′O-methyl modification, and an * is indicative of a phosphorothioate linkage between the nucleotides. Unmodified and modified versions of the guides are provided in Table 5C (Sequence Table).

CD-1 female mice, ranging 6-10 weeks of age were used in each study involving mice (n=5 per group, except TSS control n=4). Formulations were administered intravenously via tail vein injection according to the doses listed in Table 57. Animals were periodically observed for adverse effects for at least 24 hours post-dose. Six days after treatment, animals were euthanized by cardiac puncture under isoflurane anesthesia; liver tissue were collected for downstream analysis. Liver punches weighing between 5 and 15 mg were collected for isolation of genomic DNA and total RNA. Genomic DNA was extracted using a DNA isolation kit (ZymoResearch, D3010) and samples were analyzed with NGS sequencing as described in Example 1. The editing efficiency for LNPs containing the indicated gRNAs are shown in Table 57 and illustrated in FIG. 38.

TABLE 57
Mean percent editing in mouse liver.

| | Dose | C-to-T % | | | C-to-A/G % | | | Indel % | | |
|---|---|---|---|---|---|---|---|---|---|---|
| Sample | (mg/kg) | Mean | SD | n | Mean | SD | n | Mean | SD | n |
| TSS | 0 | 0.00 | 0.00 | 4 | 0.10 | 0.00 | 4 | 0.08 | 0.05 | 4 |
| SEQ ID NO: 340 + G021844 | 0.03 | 0.00 | 0.00 | 5 | 0.08 | 0.04 | 5 | 40.88 | 14.16 | 5 |
| (Nme2Cas9 + pgRNA) | 0.1 | 0.00 | 0.00 | 5 | 0.02 | 0.04 | 5 | 66.02 | 5.01 | 5 |
| SEQ ID No: 310 + SEQ ID | 0.03 | 25.60 | 5.28 | 5 | 3.50 | 0.76 | 5 | 11.14 | 2.18 | 5 |
| No: 34 + G021844 | | | | | | | | | | |
| (Nme2Cas9 base editor + | 0.1 | 46.34 | 1.53 | 5 | 5.74 | 0.33 | 5 | 13.52 | 0.90 | 5 |
| UGI + pgRNA) | | | | | | | | | | |
| SEQ ID No: 1 + SEQ ID | 0.03 | 9.28 | 2.82 | 5 | 0.94 | 0.54 | 5 | 7.34 | 1.61 | 5 |
| No: 34 + G000502 | | | | | | | | | | |
| (Spy BC22n + UGI + | 0.1 | 30.72 | 8.51 | 5 | 2.86 | 0.23 | 5 | 15.60 | 2.58 | 5 |
| sgRNA) | | | | | | | | | | |

### Example 24. C to T Deaminase Base Editing Screen

Candidate deamiinase-Cas9 fusion constructs were evaluated on efficiency of C to T conversion activity. All experimental deaminase C to T conversion activity were compared against construct BC27 encoding BE3 (Komor A C, Kim Y B, Packer M S, Zuris J A, Liu D R Programmable editing of a target base in genomic DNA without double-stranded DNA cleavage. Nature. 2016; 533(7603):420-424.) In total, 56 deaminase-Cas9 fusion constructs as mentioned in Table 58 were each evaluated in triplicate against sg005883 in a single experiment. The constructs were designed and methods performed as described in Example 1A.

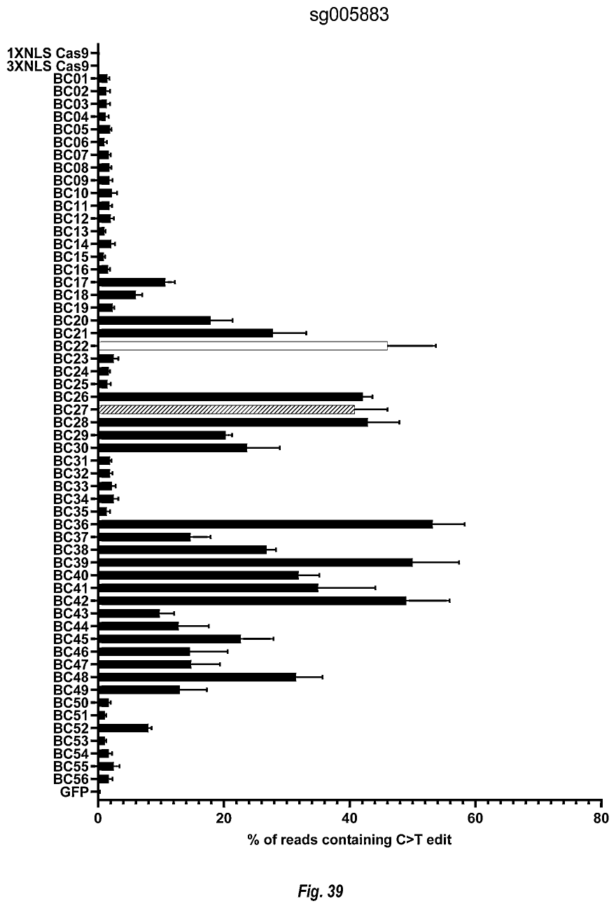

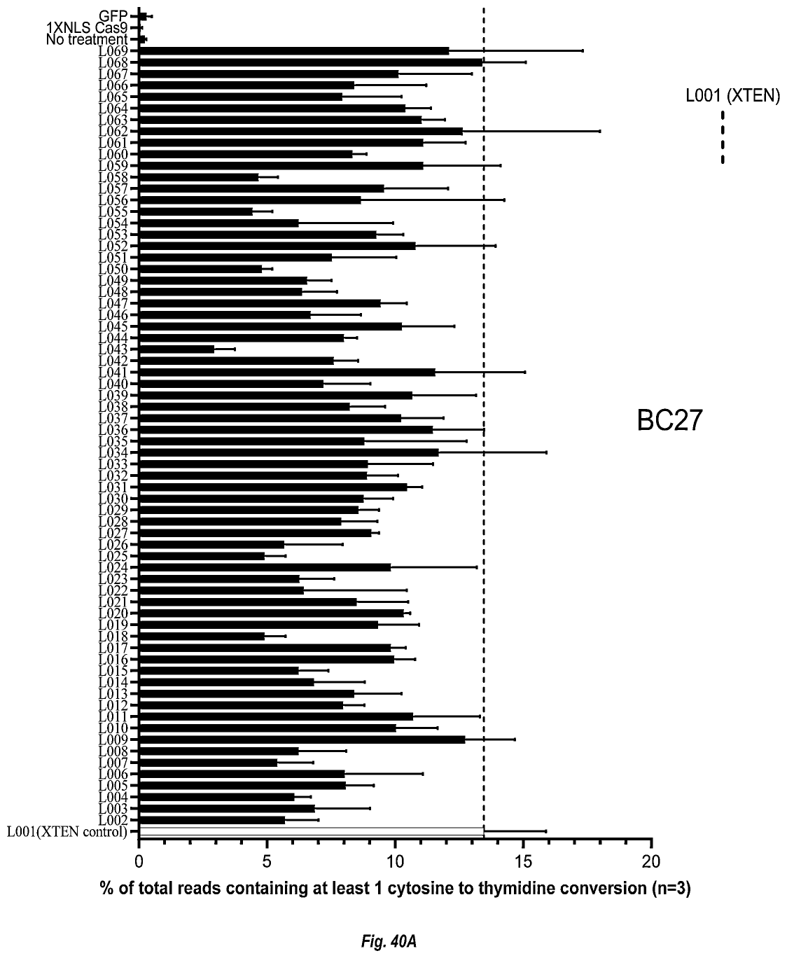

Table 59 and FIG. 39 shows the percent of total reads containing at least 1 cytosine to thymiidine conversion across all base editor constructs tested.

TABLE 58
Deaminases tested in base editor activity screens

| | | | | Amino |
|---|---|---|---|---|
| Base | | | | acid |
| Editor | UniProt for | | | sequence |
| Construct | Deaminase | Deaminase | Deaminase | (SEQ ID |
| ID | Domain | Gene Name | Species | NO) |
| BC01 | A0AON4SVL6 | Apobec1 | M. musculus | 960 |
| BC02 | A0AON4SW85 | Apobec1 | M. musculus | 961 |
| BC03 | A0QT11 | cdd | M. smegmatis | 962 |
| BC04 | Q3JJN0 | cdd | B. pseudomallei | 963 |
| BC05 | B2HDU6 | cdd | M. marinum | 964 |
| BC06 | Q81LT6 | cdd2 | B. anthracis | 965 |
| BC07 | P9WPH3 | cdd | M. tuberculosis | 966 |
| BC08 | A0A0H3MRL9 | cdd | M. leprae | 967 |
| BC09 | P19079 | cdd | B. subtilis | 968 |
| BC10 | Q06549 | CDD1 | S. cerevisiae | 969 |
| BC11 | Q81Y61 | GBAA | B. anthracis | 970 |
| BC12 | P56389 | Cda Cdd | M. musculus | 971 |
| BC13 | P32320 | CDA | H. sapiens | 972 |
| BC14 | Q12178 | FCY1 | S. cerevisiae | 973 |
| BC15 | Q22922 | cdd | C. elegans | 974 |
| BC16 | Q20628 | cdd | C. elegans | 975 |
| BC17 | Q9NRW3 | APOBEC3C | H. sapiens | 976 |
| BC18 | A0A087WX48 | Apobec3DE | H. sapiens | 977 |
| BC19 | Q82Y41 | NE0047 | N. europaea | 978 |
| BC20 | Q9GZX7 | AICDA AID | H. sapiens | 979 |
| BC21 | Q9WVE0 | Aicda Aid | M. musculus | 980 |
| BC22 | P31941 | APOBEC3A | H. sapiens | 40 |
| BC23 | Q6NTF7 | APOBEC3H | H. sapiens | 981 |
| BC24 | Q9Y235 | APOBEC2 | H. sapiens | 982 |
| BC25 | Q9WV35 | Apobec2 | M. musculus | 983 |
| BC26 | P51908 | Apobec1 | M. musculus | 984 |
| BC27 | P38483 | Apobec1 | R. norvegicus | 41 |
| BC28 | Q9TUI7 | APOBEC1 | M. domestica | 985 |
| BC29 | P41238 | APOBEC1 | H. sapiens | 986 |
| BC30 | P47855 | APOBEC1 | O. cuniculus | 987 |
| BC31 | Q8TWU6 | MK0935 | M. kandleri | 988 |
| BC32 | P0ABF6 | cdd | E. coli | 989 |
| BC33 | Q9KSM5 | cdd | V. cholerae | 990 |
| BC34 | Q3TI69 | Apobec3 | M. musculus | 991 |
| BC35 | 065896 | CDA1 | A. thalia | 992 |
| BC36 | Q7YR23 | APOBEC3G | M. mulatta | 993 |
| BC37 | Q8IUX4 | APOBEC3F | H. sapiens | 994 |
| BC38 | Q7YR25 | APOBEC3G | C. aethiops | 995 |
| BC39 | Q9UH17 | APOBEC3B | H. sapiens | 996 |
| BC40 | M1GSK9 | APOBEC3G | M. mulatta | 997 |
| BC41 | Q9HC16 | APOBEC3G | H. sapiens | 998 |
| BC42 | Q7YR24 | APOBEC3G | P. troglodytes | 999 |
| BC43 | Q96AK3 | APOBEC3D | H. sapiens | 1000 |
| BC44 | H3BJQ3 | Apobec3 | M. musculus | 1001 |
| BC45 | H3BKE5 | Apobec3 | M. musculus | 1002 |
| BC46 | E9QMH1 | Apobec3 | M. musculus | 1003 |
| BC47 | Q99J72 | Apobec3 | M. musculus | 1004 |
| BC48 | H3BJ44 | Apobec3 | M. musculus | 1005 |
| BC49 | H3BJL0 | Apobec3 | M. musculus | 1006 |
| BC50 | G3UW39 | Cdadc1 | M. musculus | 1007 |
| BC51 | P25524 | codA | E. coli | 1008 |
| BC52 | Q19Q52 | APOBEC3H | M. mulatta | 1009 |
| BC53 | Q97MB6 | CA_C0282 | C. acetobutylicum | 1010 |
| BC54 | A0A0EICHI1 | KPNJ1 | K. pneumoniae | 1011 |
| BC55 | P46087 | NOP2 | H. sapiens | 1012 |
| BC56 | Q08J23 | NSUN2 | H. sapiens | 1013 |

TABLE 59
Percent of total reads containing at least 1 cytosine to
thymidine conversion for sg005883. (n = 3)

| Construct | Mean | SD |
|---|---|---|
| 1XNLS Cas9 | 0.0 | 0.1 |
| 3XNLS Cas9 | 0.0 | 0.0 |
| BC01 | 1.5 | 0.3 |
| BC02 | 1.3 | 0.6 |
| BC03 | 1.4 | 0.5 |
| BC04 | 1.2 | 0.5 |
| BC05 | 1.9 | 0.2 |
| BC06 | 1.0 | 0.4 |
| BC07 | 1.7 | 0.3 |
| BC08 | 1.8 | 0.3 |
| BC09 | 1.8 | 0.5 |
| BC10 | 2.2 | 0.8 |
| BC11 | 1.8 | 0.4 |
| BC12 | 2.0 | 0.5 |
| BC13 | 1.0 | 0.2 |
| BC14 | 2.1 | 0.6 |
| BC15 | 0.9 | 0.2 |
| BC16 | 1.6 | 0.3 |
| BC17 | 10.7 | 1.5 |
| BC18 | 6.0 | 1.0 |
| BC18 | 6.0 | 1.0 |
| BC19 | 2.3 | 0.3 |
| BC20 | 17.9 | 3.5 |
| BC21 | 27.8 | 5.3 |
| BC22 | 46.0 | 7.7 |
| BC23 | 2.5 | 0.7 |
| BC24 | 1.7 | 0.2 |
| BC25 | 1.5 | 0.5 |
| BC26 | 42.1 | 1.5 |
| BC27 | 40.8 | 5.2 |
| BC28 | 42.9 | 5.0 |
| BC29 | 20.3 | 1.0 |
| BC30 | 23.7 | 5.2 |
| BC31 | 1.9 | 0.2 |
| BC32 | 1.9 | 0.4 |
| BC33 | 2.2 | 0.6 |
| BC34 | 2.5 | 0.7 |
| BC35 | 1.4 | 0.5 |
| BC36 | 53.2 | 5.1 |
| BC37 | 14.7 | 3.2 |
| BC38 | 26.8 | 1.5 |
| BC39 | 50.0 | 7.4 |
| BC40 | 31.9 | 3.3 |
| BC41 | 35.0 | 9.1 |
| BC42 | 49.0 | 6.9 |
| BC43 | 9.8 | 2.3 |
| BC44 | 12.8 | 4.8 |
| BC45 | 22.7 | 5.2 |
| BC46 | 14.6 | 6.0 |
| BC47 | 14.8 | 4.6 |
| BC48 | 31.5 | 4.2 |
| BC49 | 13.0 | 4.3 |
| BC50 | 1.7 | 0.3 |
| BC51 | 1.1 | 0.2 |
| BC52 | 8.0 | 0.5 |
| BC53 | 1.1 | 0.2 |
| BC54 | 1.7 | 0.5 |
| BC55 | 2.5 | 0.9 |
| BC56 | 1.7 | 0.6 |
| GFP | 0.2 | 0.1 |

### Example 25. Base Editing by Constructs with Various Linkers when Expressed by Plasmid in U-2OS Cells

A set of sixty-eight amino acid linkers listed in Table 60 encompassing various lengths and flexibilities were encoded into the region between the N-terminal cytosine deaminase and C-terminal Cas9 nickase of expression plasmid BC27 as mentioned in Example 1A. Selected base editor constructs described in Table 58 were redesigned with the substitution of the XTEN linker between the cytosine deaminase domain and the Spy Cas9 nickase domain with a linker from Table 60. The BC27 linker screen was conducted using sg001373 with target sequence UCCCUGGCUGAGGAUCCCCA (SEQ ID NO: 157). The other 10 deaminase domain linker screens were conducted using sg005883 with target sequence CCCCCCGCCGUGUUUGUGGG (SEQ ID NO: 159)

TABLE 60
Sixty-eight amino acid linkers tested in linker activity screens.

| Linker | |
|---|---|
| ID | Linker amino acid sequence |
| L001 | SGSETPGTSESATPES (SEQ ID NO: 46) |
| (XTEN | |
| control) | |
| L002 | GGS (SEQ ID NO: 211) |
| L003 | GGGGS (SEQ ID NO: 212) |
| L004 | EAAAK (SEQ ID NO: 213) |
| L005 | SEGSA (SEQ ID NO: 214) |
| L006 | SEGSAGTST (SEQ ID NO: 215) |
| L007 | GGGGSGGGGS (SEQ ID NO: 216) |
| L008 | GGGGSEAAAK (SEQ ID NO: 217) |
| L009 | EAAAKGGGGS (SEQ ID NO: 218) |
| L010 | EAAAKEAAAK (SEQ ID NO: 219) |
| L011 | SEGSAGTSTESEGSA (SEQ ID NO: 220) |
| L012 | GGGGSGGGGSGGGGS (SEQ ID NO: 221) |
| L013 | GGGGSGGGGSEAAAK (SEQ ID NO: 222) |
| L014 | GGGGSEAAAKGGGGS (SEQ ID NO: 223) |
| L015 | GGGGSEAAAKEAAAK (SEQ ID NO: 49) |
| L016 | EAAAKGGGGSGGGGS (SEQ ID NO: 50) |
| L017 | EAAAKGGGGSEAAAK (SEQ ID NO: 224) |
| L018 | EAAAKEAAAKGGGGS (SEQ ID NO: 225) |
| L019 | EAAAKEAAAKEAAAK (SEQ ID NO: 51) |
| L020 | SEGSAGTSTESEGSAGTSTE (SEQ ID NO: 226) |
| L021 | GGGGSGGGGSGGGGSGGGGS (SEQ ID NO: 52) |
| L022 | GGGGSGGGGSGGGGSEAAAK (SEQ ID NO: 227) |
| L023 | GGGGSGGGGSEAAAKGGGGS (SEQ ID NO: 228) |
| L024 | GGGGSGGGGSEAAAKEAAAK (SEQ ID NO: 53) |
| L025 | GGGGSEAAAKGGGGSGGGGS (SEQ ID NO: 54) |
| L026 | GGGGSEAAAKGGGGSEAAAK (SEQ ID NO: 229) |
| L027 | GGGGSEAAAKEAAAKGGGGS (SEQ ID NO: 230) |
| L028 | GGGGSEAAAKEAAAKEAAAK (SEQ ID NO: 231) |
| L029 | EAAAKGGGGSGGGGSGGGGS (SEQ ID NO: 232) |
| L030 | EAAAKGGGGSGGGGSEAAAK (SEQ ID NO: 233) |
| L031 | EAAAKGGGGSEAAAKGGGGS (SEQ ID NO: 234) |
| L032 | EAAAKGGGGSEAAAKEAAAK (SEQ ID NO: 235) |
| L033 | EAAAKEAAAKGGGGSGGGGS (SEQ ID NO: 236) |
| L034 | EAAAKEAAAKGGGGSEAAAK (SEQ ID NO: 237) |
| L035 | EAAAKEAAAKEAAAKGGGGS (SEQ ID NO: 238) |
| L036 | EAAAKEAAAKEAAAKEAAAK (SEQ ID NO: 56) |
| L037 | SEGSAGTSTESEGSAGTSTESEGSA (SEQ ID NO: 239) |
| L038 | GGGGSGGGGSGGGGSGGGGSGGGGS (SEQ ID NO: 240) |
| L039 | GGGGSGGGGSGGGGSGGGGSEAAAK (SEQ ID NO: 241) |
| L040 | GGGGGGGGSGGGGSEAAAKGGGGS (SEQ ID NO: 242) |
| L041 | GGGGSGGGGSGGGGSEAAAKEAAAK (SEQ ID NO: 243) |
| L042 | GGGGSGGGGSEAAAKGGGGSGGGGS (SEQ ID NO: 244) |
| L043 | GGGGSGGGGSEAAAKGGGGSEAAAK (SEQ ID NO: 245) |
| L044 | GGGGSGGGGSEAAAKEAAAKGGGGS (SEQ ID NO: 246) |
| L045 | GGGGSGGGGSEAAAKEAAAKEAAAK (SEQ ID NO: 247) |
| L046 | GGGGSEAAAKGGGGSGGGGSGGGGS (SEQ ID NO: 248) |
| L047 | GGGGSEAAAKGGGGSGGGGSEAAAK (SEQ ID NO: 249) |
| L048 | GGGGSEAAAKGGGGSEAAAKGGGGS (SEQ ID NO: 250) |
| L049 | GGGGSEAAAKGGGGSEAAAKEAAAK (SEQ ID NO: 251) |
| L050 | GGGGSEAAAKEAAAKGGGGSGGGGS (SEQ ID NO: 252) |
| L051 | GGGGSEAAAKEAAAKGGGGSEAAAK (SEQ ID NO: 57) |
| L052 | GGGGSEAAAKEAAAKEAAAKGGGGS (SEQ ID NO: 253) |
| L053 | GGGGSEAAAKEAAAKEAAAKEAAAK (SEQ ID NO: 254) |
| L054 | EAAAKGGGGSGGGGSGGGGSGGGGS (SEQ ID NO: 255) |
| L055 | EAAAKGGGGSGGGGSGGGGSEAAAK (SEQ ID NO: 256) |
| L056 | EAAAKGGGGSGGGGSEAAAKGGGGS (SEQ ID NO: 257) |
| L057 | EAAAKGGGGSGGGGSEAAAKEAAAK (SEQ ID NO: 258) |
| L058 | EAAAKGGGGSEAAAKGGGGSGGGGS (SEQ ID NO: 259) |
| L059 | EAAAKGGGGSEAAAKGGGGSEAAAK (SEQ ID NO: 260) |
| L060 | EAAAKGGGGSEAAAKEAAAKGGGGS (SEQ ID NO: 261) |
| L061 | EAAAKGGGGSEAAAKEAAAKEAAAK (SEQ ID NO: 262) |
| L062 | EAAAKEAAAKGGGGSGGGGSGGGGS (SEQ ID NO: 58) |
| L063 | EAAAKEAAAKGGGGSGGGGSEAAAK (SEQ ID NO: 59) |
| L064 | EAAAKEAAAKGGGGSEAAAKGGGGS (SEQ ID NO: 263) |
| L065 | EAAAKEAAAKGGGGSEAAAKEAAAK (SEQ ID NO: 264) |
| L066 | EAAAKEAAAKEAAAKGGGGSGGGGS (SEQ ID NO: 55) |
| L067 | EAAAKEAAAKEAAAKGGGGSEAAAK (SEQ ID NO: 265) |
| L068 | EAAAKEAAAKEAAAKEAAAKGGGGS (SEQ ID NO: 266) |
| L069 | EAAAKEAAAKEAAAKEAAAKEAAAK (SEQ ID NO: 267) |

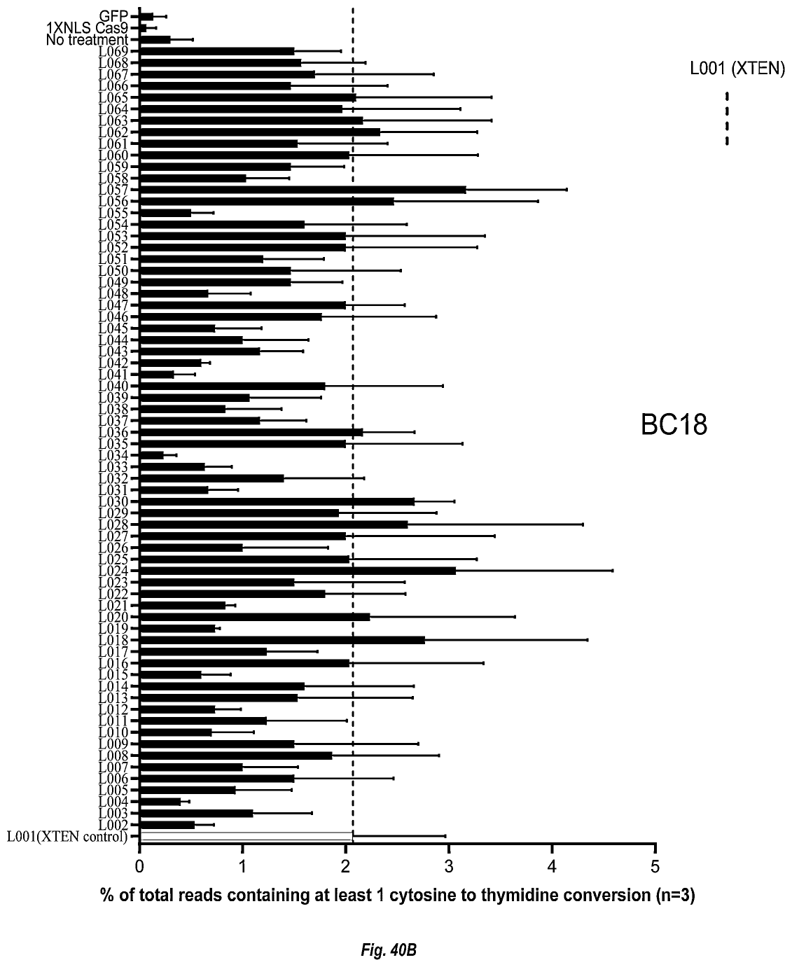

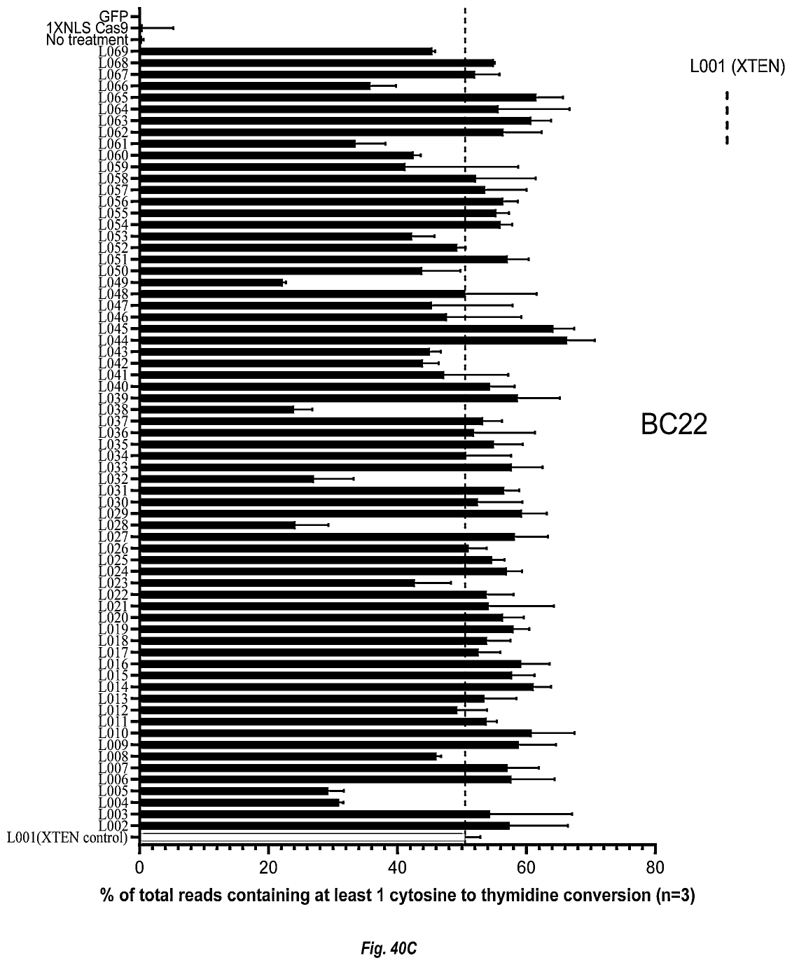

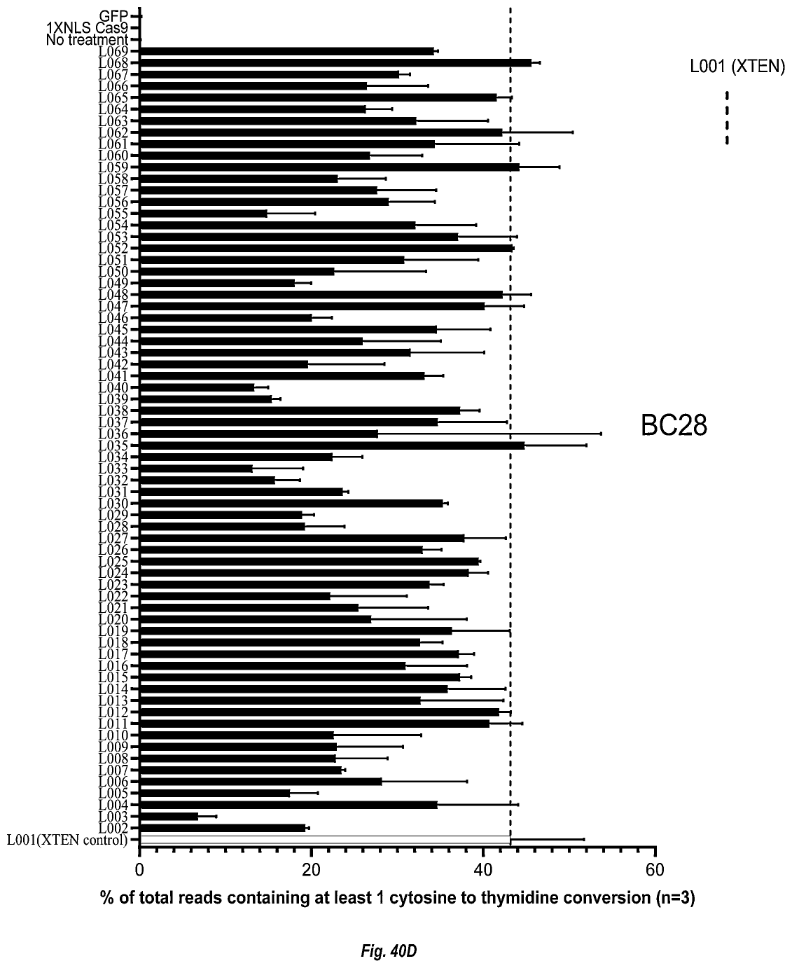

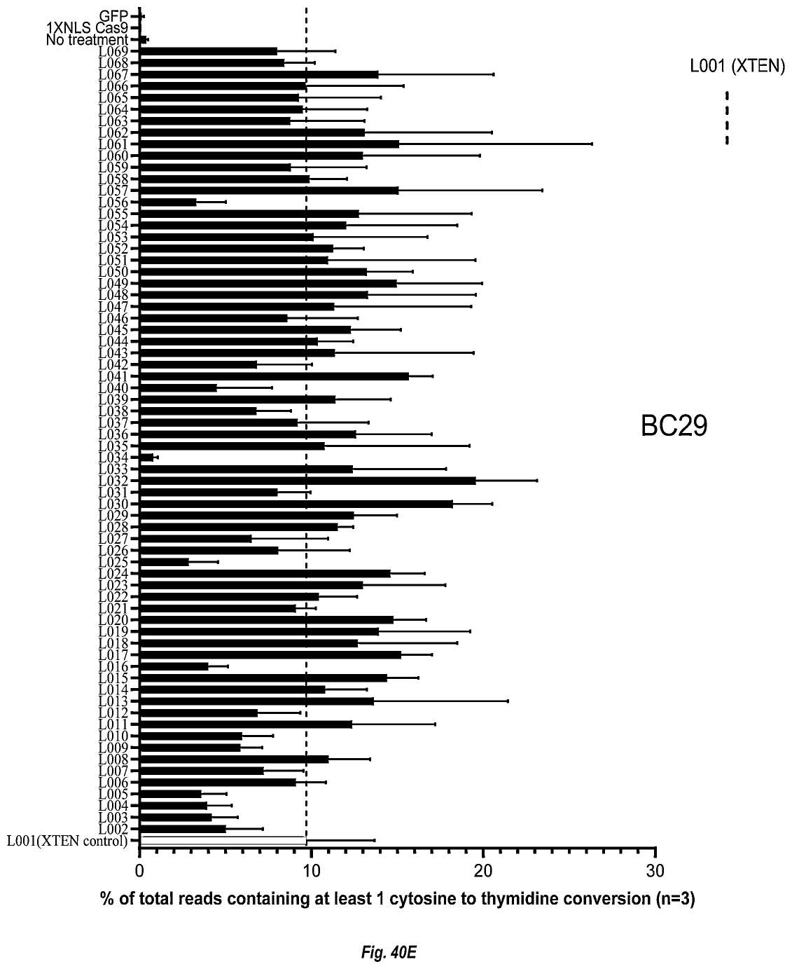

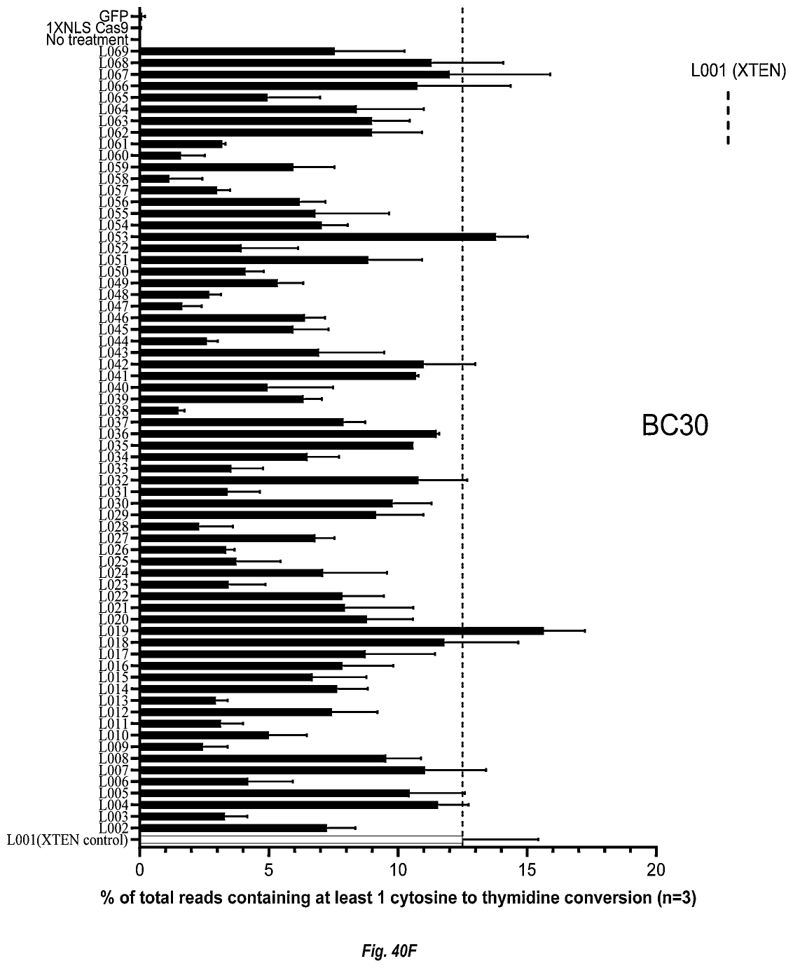

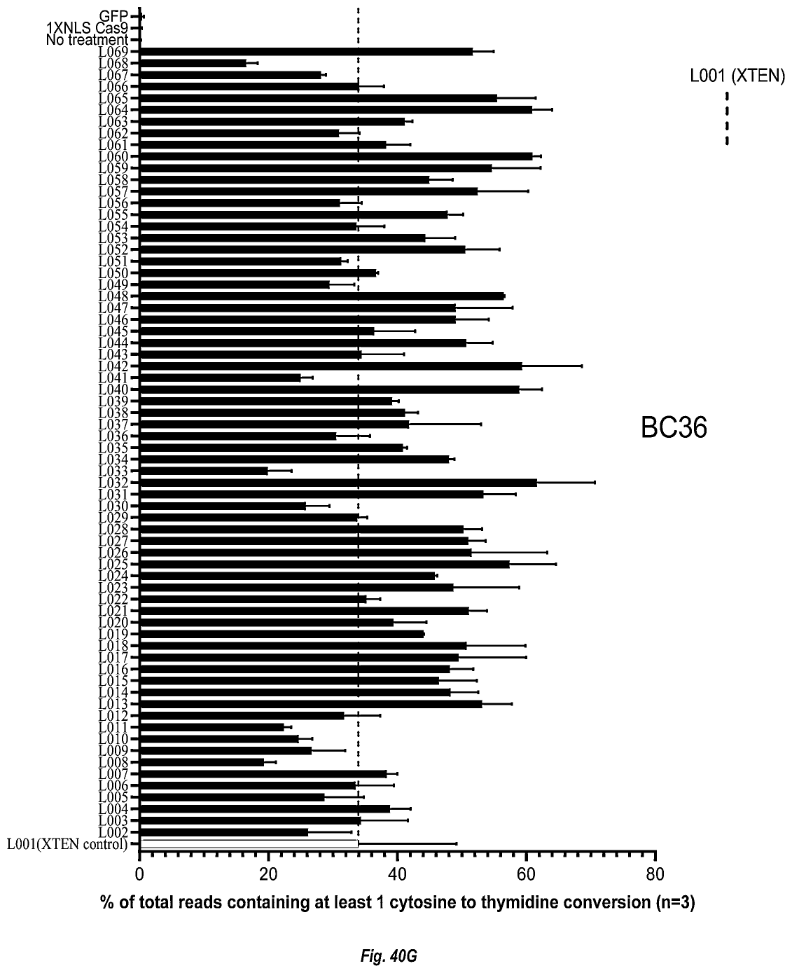

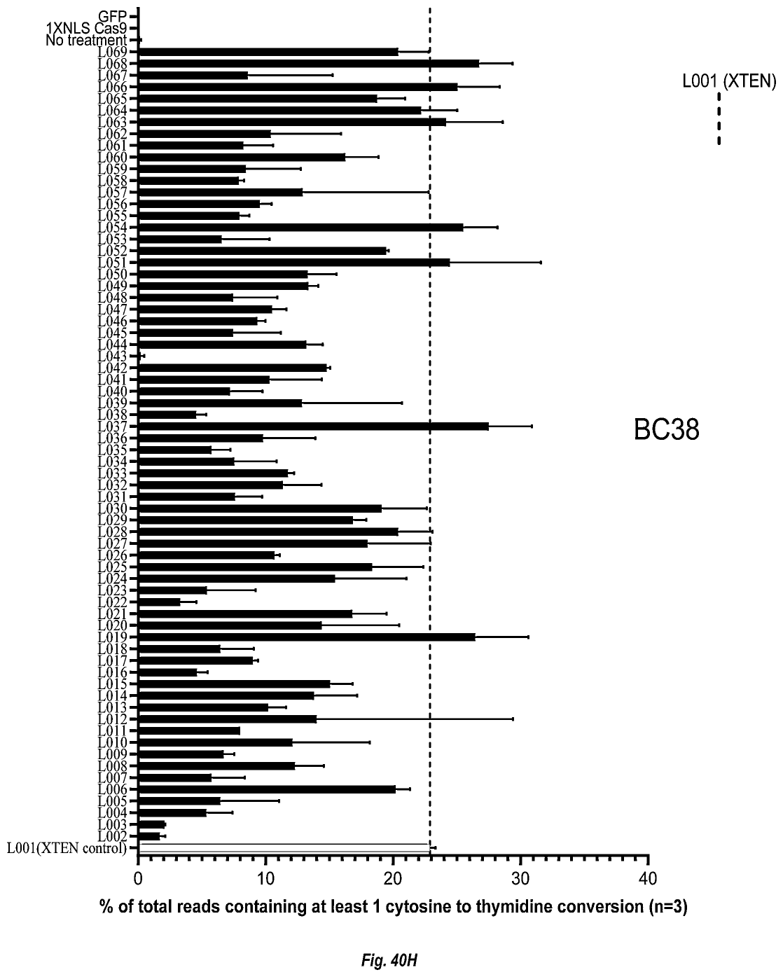

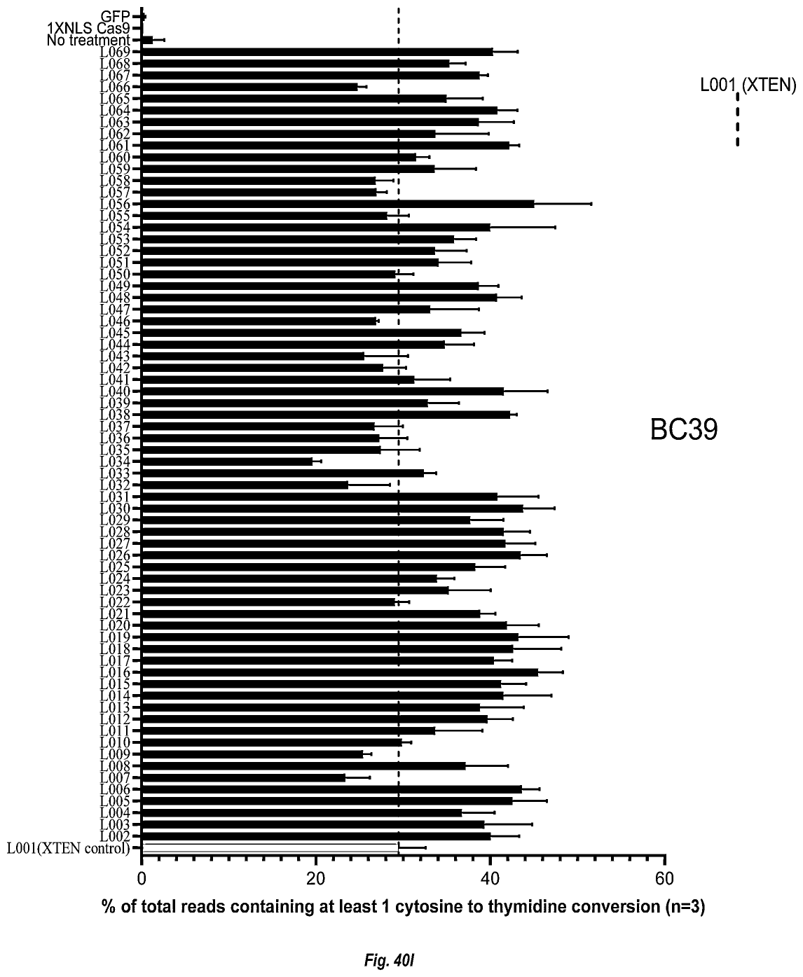

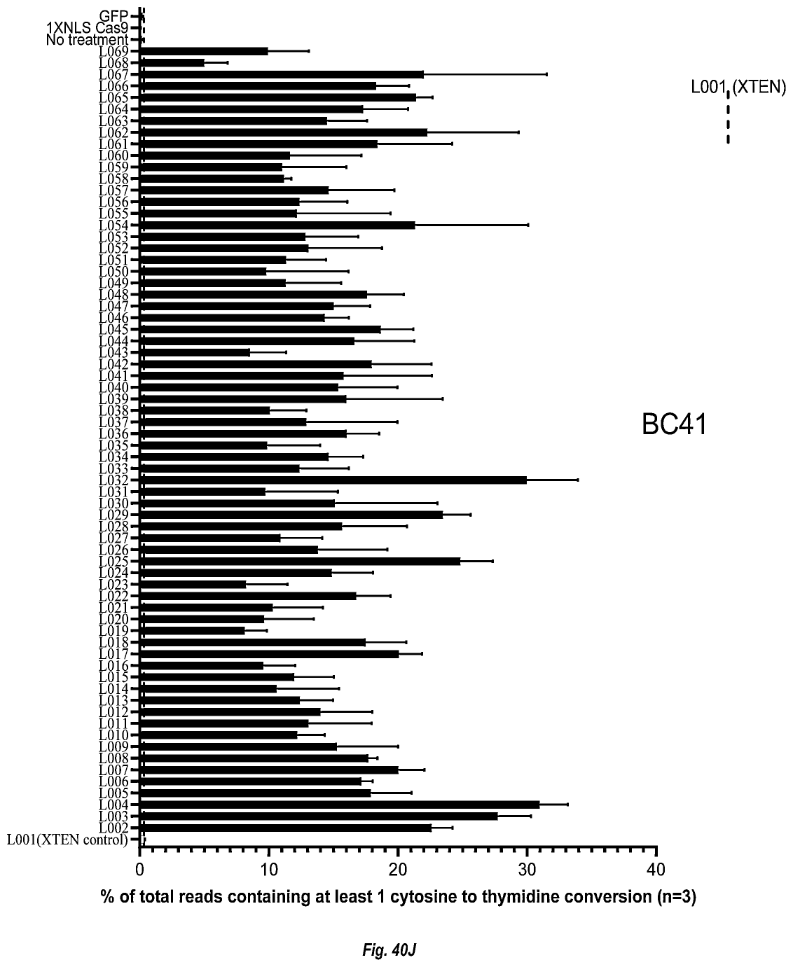

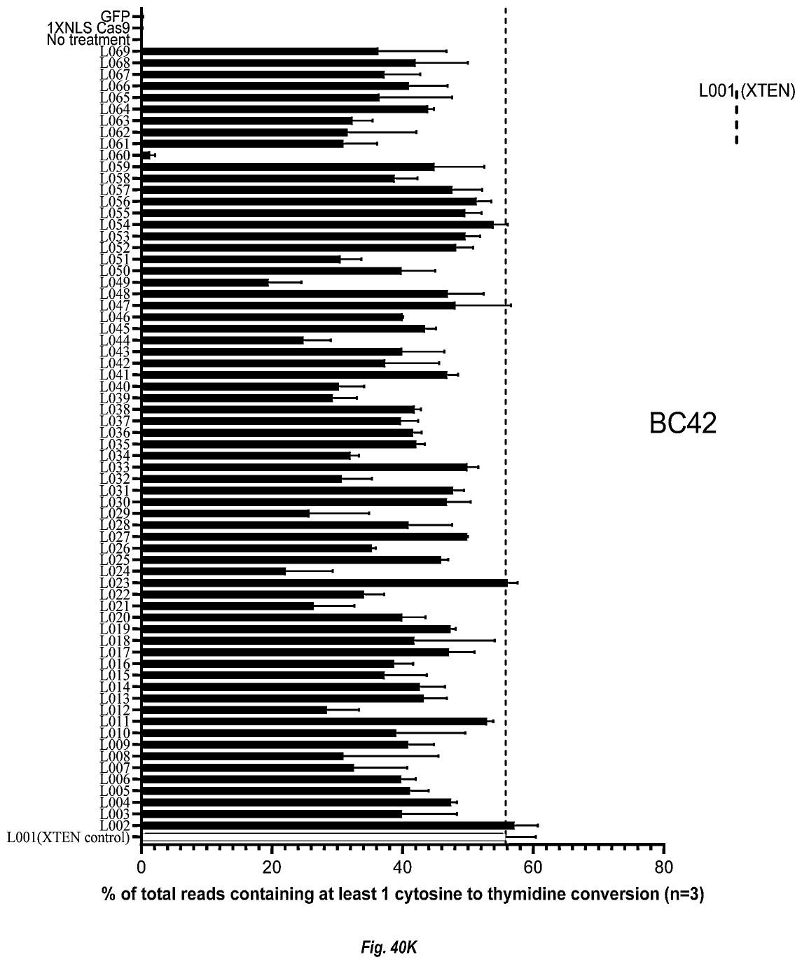

Plasmid A, uses a pUC19 backbone (GenBank® Accession Number U47119), expresses an S. pyogenes single-guide RNA (sgRNA) from a U6 promoter. Plasmid B, called pCI, expresses base editor constructs from a CMV promoter which are comprised of the candidate deamiinase fused to S. pyogenes-D10A-Cas9 by an experimental linker which is subsequently fused to one copy of UGI and one copy of SV40 NLS. U-2OS cells growing in Dulbecco's Modified Eagle's Medium (DMEM) supplemented with 10% Fetal Bovine Serum (FBS) in 96-well plates were transfected using Mirus TransIT-X2® with 100 ng each of plasmid A and plasmid B. 100 uL of fresh media was added 24 hours after initial transfection. After an additional 48 hours, the media was removed and the cells were lysed with QuickExtract™ DNA Extraction Solution (Lucigen, Cat. QE09050). Table 61A and 61B lists the percent of total reads containing at least 1 cytosine to thymidine conversion across all deaminase domains tested and representative data is shown in FIGS. 40A-40K.

TABLE 61A
Percent of total reads containing at least 1 cytosine to thymidine (% C to T) conversion

| | BC18 | | | BC22 | | | BC27 | | | BC28 | | | BC29 | | | BC30 | | |
|---|---|---|---|---|---|---|---|---|---|---|---|---|---|---|---|---|---|---|
| Linker | Mean | SD | N | Mean | SD | N | Mean | SD | N | Mean | SD | N | Mean | SD | N | Mean | SD | N |
| L001 | 2.07 | 0.90 | 3 | 50.50 | 2.36 | 3 | 13.47 | 2.42 | 3 | 43.15 | 8.56 | 2 | 9.70 | 3.97 | 3 | 12.50 | 2.94 | 3 |
| (XTEN | | | | | | | | | | | | | | | | | | |
| control) | | | | | | | | | | | | | | | | | | |
| L002 | 0.53 | 0.19 | 3 | 57.43 | 9.03 | 3 | 5.70 | 1.31 | 3 | 19.30 | 0.42 | 2 | 5.03 | 2.14 | 3 | 7.25 | 1.10 | 3 |
| L003 | 1.10 | 0.57 | 3 | 54.37 | 12.74 | 3 | 6.87 | 2.15 | 3 | 6.80 | 2.12 | 2 | 4.20 | 1.51 | 3 | 3.30 | 0.87 | 3 |
| L004 | 0.40 | 0.08 | 3 | 30.97 | 0.65 | 3 | 6.07 | 0.64 | 3 | 34.65 | 9.40 | 2 | 3.93 | 1.43 | 3 | 11.55 | 1.18 | 3 |
| L005 | 0.93 | 0.54 | 3 | 29.30 | 2.41 | 3 | 8.07 | 1.10 | 3 | 17.50 | 3.25 | 2 | 3.60 | 1.47 | 3 | 10.45 | 2.14 | 3 |
| L006 | 1.50 | 0.96 | 3 | 57.70 | 6.70 | 3 | 8.03 | 3.06 | 3 | 28.20 | 9.90 | 2 | 9.10 | 1.74 | 3 | 4.20 | 1.73 | 3 |
| L007 | 1.00 | 0.54 | 3 | 57.10 | 4.87 | 3 | 5.40 | 1.40 | 3 | 23.50 | 0.42 | 2 | 7.23 | 2.31 | 3 | 11.05 | 2.36 | 3 |
| L008 | 1.87 | 1.04 | 3 | 46.07 | 0.74 | 3 | 6.23 | 1.86 | 3 | 22.85 | 6.01 | 2 | 11.00 | 2.42 | 3 | 9.55 | 1.34 | 3 |
| L009 | 1.50 | 1.20 | 3 | 58.83 | 5.76 | 3 | 12.73 | 1.93 | 3 | 22.95 | 7.71 | 2 | 5.87 | 1.27 | 3 | 2.45 | 0.95 | 3 |
| L010 | 0.70 | 0.41 | 3 | 60.83 | 6.66 | 3 | 10.03 | 1.63 | 3 | 22.60 | 10.18 | 2 | 5.97 | 1.80 | 3 | 5.00 | 1.47 | 3 |
| L011 | 1.23 | 0.78 | 3 | 53.87 | 1.56 | 3 | 10.70 | 2.61 | 3 | 40.70 | 3.82 | 2 | 12.37 | 4.82 | 3 | 3.15 | 0.85 | 3 |
| L012 | 0.73 | 0.25 | 3 | 49.30 | 4.60 | 3 | 7.97 | 0.84 | 3 | 41.85 | 1.34 | 2 | 6.87 | 2.48 | 3 | 7.45 | 1.75 | 3 |
| L013 | 1.53 | 1.11 | 3 | 53.50 | 4.95 | 3 | 8.40 | 1.85 | 3 | 32.70 | 9.62 | 2 | 13.63 | 7.80 | 3 | 2.95 | 0.45 | 3 |
| L014 | 1.60 | 1.06 | 3 | 61.13 | 2.70 | 3 | 6.83 | 1.99 | 3 | 35.85 | 6.72 | 2 | 10.80 | 2.43 | 3 | 7.65 | 1.18 | 3 |
| L015 | 0.60 | 0.28 | 3 | 57.80 | 3.47 | 3 | 6.23 | 1.17 | 3 | 37.30 | 1.27 | 2 | 14.40 | 1.82 | 3 | 6.70 | 2.08 | 3 |
| L016 | 2.03 | 1.30 | 3 | 59.23 | 4.37 | 3 | 9.97 | 0.81 | 3 | 30.95 | 7.14 | 2 | 4.00 | 1.15 | 3 | 7.85 | 1.97 | 3 |
| L017 | 1.23 | 0.49 | 3 | 52.60 | 3.40 | 3 | 9.83 | 0.59 | 3 | 37.15 | 1.77 | 2 | 15.23 | 1.79 | 3 | 8.75 | 2.68 | 3 |
| L018 | 2.77 | 1.58 | 3 | 53.93 | 3.61 | 3 | 4.90 | 0.82 | 3 | 32.65 | 2.62 | 2 | 12.70 | 5.78 | 3 | 11.80 | 2.86 | 3 |
| L019 | 0.73 | 0.05 | 3 | 58.03 | 2.42 | 3 | 9.33 | 1.61 | 3 | 36.35 | 6.72 | 2 | 13.93 | 5.31 | 3 | 15.65 | 1.59 | 3 |
| L020 | 2.23 | 1.41 | 3 | 56.40 | 3.21 | 3 | 10.33 | 0.25 | 3 | 26.95 | 11.10 | 2 | 14.77 | 1.90 | 3 | 8.80 | 1.78 | 3 |
| L021 | 0.83 | 0.09 | 3 | 54.17 | 10.11 | 3 | 8.50 | 2.01 | 3 | 25.45 | 8.13 | 2 | 9.10 | 1.15 | 3 | 7.95 | 2.65 | 3 |
| L022 | 1.80 | 0.78 | 3 | 53.87 | 4.14 | 3 | 6.43 | 4.02 | 3 | 22.20 | 8.91 | 2 | 10.43 | 2.23 | 3 | 7.85 | 1.60 | 3 |
| L023 | 1.50 | 1.07 | 3 | 42.73 | 5.58 | 3 | 6.27 | 1.36 | 3 | 33.75 | 1.63 | 2 | 13.00 | 4.80 | 3 | 3.45 | 1.42 | 3 |
| L024 | 3.07 | 1.52 | 3 | 57.00 | 2.35 | 3 | 9.83 | 3.35 | 3 | 38.30 | 2.26 | 2 | 14.60 | 1.99 | 3 | 7.10 | 2.48 | 3 |
| L025 | 2.03 | 1.24 | 3 | 54.70 | 1.92 | 3 | 4.90 | 0.82 | 3 | 39.45 | 0.21 | 2 | 2.87 | 1.70 | 3 | 3.75 | 1.70 | 3 |
| L026 | 1.00 | 0.83 | 3 | 51.07 | 2.79 | 3 | 5.67 | 2.28 | 3 | 32.95 | 2.19 | 2 | 8.07 | 4.16 | 3 | 3.35 | 0.32 | 3 |
| L027 | 2.00 | 1.44 | 3 | 58.23 | 5.15 | 3 | 9.07 | 0.31 | 3 | 37.80 | 4.81 | 2 | 6.53 | 4.43 | 3 | 6.80 | 0.74 | 3 |
| L028 | 2.60 | 1.70 | 3 | 24.20 | 5.10 | 3 | 7.90 | 1.41 | 3 | 19.25 | 4.60 | 2 | 11.53 | 0.90 | 3 | 2.30 | 1.31 | 3 |
| L029 | 1.93 | 0.95 | 3 | 59.35 | 3.82 | 3 | 8.57 | 0.80 | 3 | 18.90 | 1.41 | 2 | 12.47 | 2.51 | 3 | 9.15 | 1.84 | 3 |
| L030 | 2.67 | 0.39 | 3 | 52.50 | 6.90 | 3 | 8.77 | 1.15 | 3 | 35.30 | 0.57 | 2 | 18.23 | 2.30 | 3 | 9.80 | 1.50 | 3 |
| L031 | 0.67 | 0.29 | 3 | 56.57 | 2.35 | 3 | 10.47 | 0.59 | 3 | 23.65 | 0.64 | 2 | 8.03 | 1.91 | 3 | 3.40 | 1.25 | 3 |
| L032 | 1.40 | 0.78 | 3 | 27.07 | 6.12 | 3 | 8.90 | 1.22 | 3 | 15.75 | 2.90 | 2 | 19.57 | 3.56 | 3 | 10.80 | 1.88 | 3 |
| L033 | 0.63 | 0.26 | 3 | 57.75 | 4.76 | 3 | 8.93 | 2.55 | 3 | 13.15 | 5.87 | 2 | 12.40 | 5.44 | 3 | 3.55 | 1.23 | 3 |
| L034 | 0.23 | 0.12 | 3 | 50.70 | 6.94 | 3 | 11.70 | 4.20 | 3 | 22.45 | 3.46 | 2 | 0.80 | 0.26 | 3 | 6.50 | 1.22 | 3 |
| L035 | 2.00 | 1.13 | 3 | 55.00 | 4.45 | 3 | 8.80 | 3.99 | 3 | 44.80 | 7.21 | 2 | 10.77 | 8.43 | 3 | 10.60 | — | 1 |
| L036 | 2.17 | 0.50 | 3 | 51.90 | 9.44 | 3 | 11.47 | 2.01 | 3 | 27.75 | 25.95 | 2 | 12.60 | 4.39 | 3 | 11.50 | 0.10 | 3 |
| L037 | 1.17 | 0.45 | 3 | 53.30 | 2.92 | 3 | 10.23 | 1.65 | 3 | 34.70 | 8.06 | 2 | 9.20 | 4.12 | 3 | 7.90 | 0.83 | 3 |
| L038 | 0.83 | 0.54 | 3 | 23.97 | 2.82 | 3 | 8.23 | 1.38 | 3 | 37.30 | 2.26 | 2 | 6.80 | 2.01 | 3 | 1.50 | 0.23 | 3 |
| L039 | 1.07 | 0.69 | 3 | 58.70 | 6.50 | 3 | 10.67 | 2.49 | 3 | 15.40 | 0.99 | 2 | 11.40 | 3.21 | 3 | 6.35 | 0.71 | 3 |
| L040 | 1.80 | 1.14 | 3 | 54.37 | 3.81 | 3 | 7.20 | 1.83 | 3 | 13.35 | 1.63 | 2 | 4.50 | 3.22 | 3 | 4.95 | 2.54 | 3 |
| L041 | 0.33 | 0.21 | 3 | 47.27 | 9.92 | 3 | 11.57 | 3.50 | 3 | 33.15 | 2.19 | 2 | 15.67 | 1.39 | 3 | 10.70 | 0.10 | 3 |
| L042 | 0.60 | 0.08 | 3 | 43.97 | 2.45 | 3 | 7.60 | 0.95 | 3 | 19.60 | 8.91 | 2 | 6.83 | 3.21 | 3 | 11.00 | 1.99 | 3 |
| L043 | 1.17 | 0.42 | 3 | 45.07 | 1.69 | 3 | 2.93 | 0.81 | 3 | 31.55 | 8.56 | 2 | 11.37 | 8.06 | 3 | 6.95 | 2.52 | 3 |
| L044 | 1.00 | 0.64 | 3 | 66.35 | 4.26 | 3 | 8.00 | 0.52 | 3 | 25.95 | 9.12 | 2 | 10.37 | 2.06 | 3 | 2.60 | 0.42 | 3 |
| L045 | 0.73 | 0.45 | 3 | 64.20 | 3.24 | 3 | 10.27 | 2.05 | 3 | 34.60 | 6.22 | 2 | 12.30 | 2.90 | 3 | 5.95 | 1.36 | 3 |
| L046 | 1.77 | 1.11 | 3 | 47.70 | 11.53 | 3 | 6.70 | 1.97 | 3 | 20.05 | 2.33 | 2 | 8.60 | 4.10 | 3 | 6.40 | 0.78 | 3 |
| L047 | 2.00 | 0.57 | 3 | 45.37 | 12.50 | 3 | 9.43 | 1.02 | 3 | 40.15 | 4.60 | 2 | 11.33 | 7.96 | 3 | 1.65 | 0.75 | 3 |
| L048 | 0.67 | 0.41 | 3 | 50.53 | 11.07 | 3 | 6.37 | 1.37 | 3 | 42.25 | 3.32 | 2 | 13.30 | 6.26 | 3 | 2.70 | 0.45 | 3 |
| L049 | 1.47 | 0.50 | 3 | 22.23 | 0.48 | 3 | 6.57 | 0.95 | 3 | 18.05 | 1.91 | 2 | 14.97 | 4.96 | 3 | 5.35 | 0.98 | 3 |
| L050 | 1.47 | 1.07 | 3 | 43.87 | 5.89 | 3 | 4.80 | 0.40 | 3 | 22.65 | 10.68 | 2 | 13.23 | 2.67 | 3 | 4.10 | 0.70 | 3 |
| L051 | 1.20 | 0.59 | 3 | 57.13 | 3.24 | 3 | 7.53 | 2.51 | 3 | 30.80 | 8.63 | 2 | 10.97 | 8.58 | 3 | 8.85 | 2.08 | 3 |
| L052 | 2.00 | 1.28 | 3 | 49.30 | 1.31 | 3 | 10.80 | 3.12 | 3 | 43.40 | 0.14 | 2 | 11.27 | 1.78 | 3 | 3.95 | 2.18 | 3 |
| L053 | 2.00 | 1.35 | 3 | 42.30 | 3.48 | 3 | 9.27 | 1.05 | 3 | 37.05 | 6.86 | 2 | 10.13 | 6.62 | 3 | 13.80 | 1.23 | 3 |
| L054 | 1.60 | 0.99 | 3 | 56.00 | 1.80 | 3 | 6.23 | 3.70 | 3 | 32.10 | 7.07 | 2 | 12.03 | 6.46 | 3 | 7.05 | 1.00 | 3 |
| L055 | 0.50 | 0.22 | 3 | 55.37 | 1.92 | 3 | 4.43 | 0.78 | 3 | 14.85 | 5.59 | 2 | 12.77 | 6.56 | 3 | 6.80 | 2.86 | 3 |
| L056 | 2.47 | 1.40 | 3 | 56.43 | 2.27 | 3 | 8.67 | 5.59 | 3 | 29.00 | 5.37 | 2 | 3.30 | 1.73 | 3 | 6.20 | 0.99 | 3 |
| L057 | 3.17 | 0.97 | 3 | 53.63 | 6.42 | 3 | 9.57 | 2.49 | 3 | 27.65 | 6.86 | 2 | 15.07 | 8.36 | 3 | 3.00 | 0.50 | 3 |
| L058 | 1.03 | 0.42 | 3 | 52.20 | 9.24 | 3 | 4.67 | 0.76 | 3 | 23.05 | 5.59 | 2 | 9.90 | 2.18 | 3 | 1.15 | 1.27 | 3 |
| L059 | 1.47 | 0.52 | 3 | 41.27 | 17.48 | 3 | 11.10 | 3.01 | 3 | 44.20 | 4.67 | 2 | 8.80 | 4.41 | 3 | 5.95 | 1.59 | 3 |
| L060 | 2.03 | 1.25 | 3 | 42.53 | 1.09 | 3 | 8.33 | 0.55 | 3 | 26.80 | 6.08 | 2 | 13.00 | 6.80 | 3 | 1.60 | 0.92 | 3 |
| L061 | 1.53 | 0.87 | 3 | 33.53 | 4.62 | 3 | 11.10 | 1.65 | 3 | 34.35 | 9.83 | 2 | 15.10 | 11.23 | 3 | 3.20 | 0.12 | 3 |
| L062 | 2.33 | 0.94 | 3 | 56.47 | 5.91 | 3 | 12.63 | 5.36 | 3 | 42.20 | 8.20 | 2 | 13.10 | 7.41 | 3 | 9.00 | 1.93 | 3 |
| L063 | 2.17 | 1.25 | 3 | 60.77 | 3.09 | 3 | 11.03 | 0.91 | 3 | 32.20 | 8.34 | 2 | 8.77 | 4.32 | 3 | 9.00 | 1.45 | 3 |
| L064 | 1.97 | 1.14 | 3 | 55.70 | 10.99 | 3 | 10.40 | 1.00 | 3 | 26.35 | 3.04 | 2 | 9.50 | 3.74 | 3 | 8.40 | 2.60 | 3 |
| L065 | 2.10 | 1.31 | 3 | 61.57 | 4.12 | 3 | 7.93 | 2.32 | 3 | 41.55 | 1.77 | 2 | 9.27 | 4.77 | 3 | 4.95 | 2.04 | 3 |
| L066 | 1.47 | 0.94 | 3 | 35.83 | 3.98 | 3 | 8.40 | 2.82 | 3 | 26.45 | 7.14 | 2 | 9.67 | 5.69 | 3 | 10.75 | 3.61 | 3 |
| L067 | 1.70 | 1.15 | 3 | 52.07 | 3.77 | 3 | 10.13 | 2.87 | 3 | 30.20 | 1.27 | 2 | 13.90 | 6.69 | 3 | 12.00 | 3.89 | 3 |
| L068 | 1.57 | 0.62 | 3 | 54.97 | 0.16 | 3 | 13.40 | 1.71 | 3 | 45.60 | 0.99 | 2 | 8.43 | 1.76 | 3 | 11.30 | 2.78 | 3 |
| L069 | 1.50 | 0.45 | 3 | 45.43 | 0.45 | 3 | 12.10 | 5.22 | 3 | 34.25 | 0.49 | 2 | 8.00 | 3.39 | 3 | 7.55 | 2.70 | 3 |
| No | 0.30 | 0.22 | 3 | 0.40 | 0.22 | 3 | 0.23 | 0.06 | 3 | 0.05 | 0.07 | 2 | 0.40 | 0.10 | 3 | 0.00 | 0.00 | 3 |
| treatment | | | | | | | | | | | | | | | | | | |
| 1XNLS | 0.07 | 0.09 | 3 | 0.50 | 4.77 | 3 | 0.07 | 0.06 | 0 | 0.00 | 0.00 | 2 | 0.10 | 0.00 | 3 | 0.00 | 0.06 | 3 |
| Cas9 | | | | | | | | | | | | | | | | | | |
| GFP | 0.13 | 0.12 | 3 | | | 3 | 0.30 | 0.20 | 1 | 0.10 | 0.14 | 2 | 0.13 | 0.12 | 3 | 0.10 | 0.10 | 3 |

TABLE 61B
Mean percent of total reads containing at least 1 cytosine to thymidine (% C to T) conversion

| | BC36 | | | BC38 | | | BC39 | | | BC41 | | | BC42 | | |
|---|---|---|---|---|---|---|---|---|---|---|---|---|---|---|---|
| Linker | Mean | SD | N | Mean | SD | N | Mean | SD | N | Mean | SD | N | Mean | SD | N |
| L001 (XTEN | 33.95 | 15.20 | 2 | 22.90 | 0.42 | 2 | 29.47 | 3.11 | 3 | 0.33 | 0.09 | 3 | 55.80 | 4.60 | 3 |
| control) | | | | | | | | | | | | | | | |
| L002 | 26.15 | 6.72 | 2 | 1.70 | 0.42 | 2 | 40.03 | 3.27 | 3 | 22.60 | 1.63 | 3 | 57.15 | 3.55 | 3 |
| L003 | 34.35 | 7.28 | 2 | 2.05 | 0.07 | 2 | 39.30 | 5.48 | 3 | 27.73 | 2.58 | 3 | 39.90 | 8.40 | 3 |
| L004 | 38.85 | 3.18 | 2 | 5.35 | 2.05 | 2 | 36.73 | 3.75 | 3 | 30.97 | 2.17 | 3 | 47.45 | 0.85 | 3 |
| L005 | 28.70 | 6.08 | 2 | 6.45 | 4.60 | 2 | 42.53 | 3.93 | 3 | 17.90 | 3.16 | 3 | 41.15 | 2.85 | 3 |
| L006 | 33.50 | 5.94 | 2 | 20.20 | 1.13 | 2 | 43.60 | 2.02 | 3 | 17.17 | 0.88 | 3 | 39.80 | 2.20 | 3 |
| L007 | 38.35 | 1.63 | 2 | 5.75 | 2.62 | 2 | 23.37 | 2.80 | 3 | 20.03 | 2.01 | 3 | 32.55 | 8.15 | 3 |
| L008 | 19.30 | 1.84 | 2 | 12.30 | 2.26 | 2 | 37.15 | 4.88 | 3 | 17.70 | 0.71 | 3 | 30.95 | 14.55 | 3 |
| L009 | 26.70 | 5.23 | 2 | 6.70 | 0.85 | 2 | 25.40 | 0.96 | 3 | 15.27 | 4.74 | 3 | 40.85 | 3.95 | 3 |
| L010 | 24.70 | 2.12 | 2 | 12.10 | 6.08 | 2 | 29.85 | 1.06 | 3 | 12.20 | 2.13 | 3 | 39.05 | 10.55 | 3 |
| L011 | 22.40 | 1.13 | 2 | 8.00 | 0.00 | 2 | 33.67 | 5.40 | 3 | 13.07 | 4.90 | 3 | 52.95 | 0.95 | 3 |
| L012 | 31.75 | 5.59 | 2 | 14.00 | 15.41 | 2 | 39.67 | 2.90 | 3 | 14.00 | 4.01 | 3 | 28.40 | 4.90 | 3 |
| L013 | 53.15 | 4.60 | 2 | 10.20 | 1.41 | 2 | 38.80 | 5.02 | 3 | 12.40 | 2.57 | 3 | 43.25 | 3.55 | 3 |
| L014 | 48.25 | 4.31 | 2 | 13.80 | 3.39 | 2 | 41.50 | 5.51 | 3 | 10.60 | 4.83 | 3 | 42.65 | 3.85 | 3 |
| L015 | 46.45 | 5.87 | 2 | 15.05 | 1.77 | 2 | 41.23 | 2.85 | 3 | 11.97 | 3.06 | 3 | 37.25 | 6.45 | 3 |
| L016 | 48.15 | 3.61 | 2 | 4.60 | 0.85 | 2 | 45.47 | 2.84 | 3 | 9.57 | 2.47 | 3 | 38.70 | 2.90 | 3 |
| L017 | 49.50 | 10.47 | 2 | 9.00 | 0.42 | 2 | 40.40 | 2.10 | 3 | 20.07 | 1.80 | 3 | 47.10 | 3.90 | 3 |
| L018 | 50.75 | 9.12 | 2 | 6.45 | 2.62 | 2 | 42.60 | 5.54 | 3 | 17.50 | 3.15 | 3 | 41.80 | 12.30 | 3 |
| L019 | 44.05 | 0.07 | 2 | 26.45 | 4.17 | 2 | 43.20 | 5.78 | 3 | 8.13 | 1.72 | 3 | 47.35 | 0.75 | 3 |
| L020 | 39.40 | 5.09 | 2 | 14.40 | 6.08 | 2 | 41.90 | 3.65 | 3 | 9.63 | 3.84 | 3 | 39.95 | 3.55 | 3 |
| L021 | 51.10 | 2.83 | 2 | 16.80 | 2.69 | 2 | 38.83 | 1.74 | 3 | 10.30 | 3.89 | 3 | 26.40 | 6.20 | 3 |
| L022 | 35.20 | 2.12 | 2 | 3.30 | 1.27 | 2 | 29.05 | 1.63 | 3 | 16.77 | 2.65 | 3 | 34.10 | 3.10 | 3 |
| L023 | 48.70 | 10.18 | 2 | 5.40 | 3.82 | 2 | 35.23 | 4.81 | 3 | 8.23 | 3.21 | 3 | 56.10 | 1.50 | 3 |
| L024 | 45.80 | 0.42 | 2 | 15.45 | 5.59 | 2 | 33.87 | 2.02 | 3 | 14.87 | 3.20 | 3 | 22.10 | 7.20 | 3 |
| L025 | 57.40 | 7.21 | 2 | 18.35 | 4.03 | 2 | 38.27 | 3.41 | 3 | 24.83 | 2.50 | 3 | 45.90 | 1.10 | 3 |
| L026 | 51.50 | 11.74 | 2 | 10.70 | 0.42 | 2 | 43.47 | 3.01 | 3 | 13.80 | 5.37 | 3 | 35.30 | 0.60 | 3 |
| L027 | 51.00 | 2.69 | 2 | 18.00 | 4.95 | 2 | 41.73 | 3.43 | 3 | 10.90 | 3.24 | 3 | 49.90 | 0.10 | 3 |
| L028 | 50.25 | 2.90 | 2 | 20.40 | 2.69 | 2 | 41.53 | 3.00 | 3 | 15.67 | 5.03 | 3 | 40.90 | 6.70 | 3 |
| L029 | 33.80 | 1.56 | 2 | 16.85 | 1.06 | 2 | 37.70 | 3.80 | 3 | 23.47 | 2.16 | 3 | 25.75 | 9.15 | 3 |
| L030 | 25.80 | 3.68 | 2 | 19.10 | 3.54 | 2 | 43.77 | 3.59 | 3 | 15.10 | 7.97 | 3 | 46.80 | 3.60 | 3 |
| L031 | 53.35 | 5.02 | 2 | 7.60 | 2.12 | 2 | 40.80 | 4.72 | 3 | 9.73 | 5.61 | 3 | 47.75 | 1.65 | 3 |
| L032 | 61.65 | 8.98 | 2 | 11.35 | 3.04 | 2 | 23.70 | 4.80 | 3 | 29.97 | 3.97 | 3 | 30.70 | 4.60 | 3 |
| L033 | 19.90 | 3.68 | 2 | 11.75 | 0.49 | 2 | 32.37 | 1.42 | 3 | 12.37 | 3.82 | 3 | 49.95 | 1.65 | 3 |
| L034 | 48.00 | 0.85 | 2 | 7.55 | 3.32 | 2 | 19.60 | 0.99 | 3 | 14.63 | 2.67 | 3 | 32.05 | 1.25 | 3 |
| L035 | 40.85 | 0.64 | 2 | 5.75 | 1.48 | 2 | 27.43 | 4.48 | 3 | 9.87 | 4.11 | 3 | 42.10 | 1.30 | 3 |
| L036 | 30.50 | 5.23 | 2 | 9.80 | 4.10 | 2 | 27.27 | 3.21 | 3 | 16.03 | 2.52 | 3 | 41.60 | 1.30 | 3 |
| L037 | 41.80 | 11.17 | 2 | 27.50 | 3.39 | 2 | 26.73 | 3.25 | 3 | 12.90 | 7.05 | 3 | 39.75 | 2.65 | 3 |
| L038 | 41.20 | 1.98 | 2 | 4.55 | 0.78 | 2 | 42.27 | 0.76 | 3 | 10.07 | 2.83 | 3 | 41.85 | 0.95 | 3 |
| L039 | 39.20 | 0.99 | 2 | 12.85 | 7.85 | 2 | 32.83 | 3.56 | 3 | 16.00 | 7.47 | 3 | 29.30 | 3.70 | 3 |
| L040 | 58.90 | 3.54 | 2 | 7.20 | 2.55 | 2 | 41.53 | 5.04 | 3 | 15.37 | 4.58 | 3 | 30.25 | 3.85 | 3 |
| L041 | 25.00 | 1.84 | 2 | 10.30 | 4.10 | 2 | 31.27 | 4.11 | 3 | 15.80 | 6.83 | 3 | 46.85 | 1.65 | 3 |
| L042 | 59.35 | 9.26 | 2 | 14.80 | 0.28 | 2 | 27.70 | 2.63 | 3 | 17.97 | 4.63 | 3 | 37.35 | 8.25 | 3 |
| L043 | 34.45 | 6.58 | 2 | 0.20 | 0.28 | 2 | 25.53 | 5.01 | 3 | 8.53 | 2.82 | 3 | 39.95 | 6.45 | 3 |
| L044 | 50.70 | 4.10 | 2 | 13.20 | 1.27 | 2 | 34.77 | 3.35 | 3 | 16.63 | 4.64 | 3 | 24.85 | 4.15 | 3 |
| L045 | 36.40 | 6.36 | 2 | 7.45 | 3.75 | 2 | 36.67 | 2.66 | 3 | 18.67 | 2.53 | 3 | 43.45 | 1.65 | 3 |
| L046 | 49.05 | 5.16 | 2 | 9.35 | 0.64 | 2 | 26.90 | 0.28 | 3 | 14.33 | 1.87 | 3 | 40.00 | 0.10 | 3 |
| L047 | 49.05 | 8.84 | 2 | 10.50 | 1.13 | 2 | 33.10 | 5.60 | 3 | 15.03 | 2.81 | 3 | 48.10 | 8.50 | 3 |
| L048 | 56.50 | 0.14 | 2 | 7.45 | 3.46 | 2 | 40.77 | 2.81 | 3 | 17.60 | 2.84 | 3 | 46.95 | 5.45 | 3 |
| L049 | 29.50 | 3.82 | 2 | 13.35 | 0.78 | 2 | 38.67 | 2.25 | 3 | 11.30 | 4.30 | 3 | 19.50 | 5.00 | 3 |
| L050 | 36.70 | 0.28 | 2 | 13.30 | 2.26 | 2 | 29.10 | 2.08 | 3 | 9.80 | 6.38 | 3 | 39.85 | 5.15 | 3 |
| L051 | 31.30 | 0.99 | 2 | 24.45 | 7.14 | 2 | 34.07 | 3.72 | 3 | 11.33 | 3.11 | 3 | 30.50 | 3.20 | 3 |
| L052 | 50.55 | 5.30 | 2 | 19.45 | 0.21 | 2 | 33.63 | 3.65 | 3 | 13.07 | 5.70 | 3 | 48.20 | 2.60 | 3 |
| L053 | 44.35 | 4.60 | 2 | 6.55 | 3.75 | 2 | 35.83 | 2.54 | 3 | 12.83 | 4.09 | 3 | 49.65 | 2.25 | 3 |
| L054 | 33.65 | 4.31 | 2 | 25.50 | 2.69 | 2 | 39.97 | 7.47 | 3 | 21.33 | 8.77 | 3 | 53.95 | 2.15 | 3 |
| L055 | 47.80 | 2.40 | 2 | 7.95 | 0.78 | 2 | 28.17 | 2.50 | 3 | 12.17 | 7.25 | 3 | 49.60 | 2.50 | 3 |
| L056 | 31.10 | 3.39 | 2 | 9.55 | 0.92 | 2 | 45.03 | 6.52 | 3 | 12.37 | 3.70 | 3 | 51.35 | 2.25 | 3 |
| L057 | 52.45 | 7.85 | 2 | 12.90 | 9.90 | 2 | 26.93 | 1.20 | 3 | 14.63 | 5.10 | 3 | 47.65 | 4.55 | 3 |
| L058 | 44.95 | 3.61 | 2 | 7.90 | 0.42 | 2 | 26.85 | 2.05 | 3 | 11.17 | 0.57 | 3 | 38.80 | 3.50 | 3 |
| L059 | 54.65 | 7.57 | 2 | 8.45 | 4.31 | 2 | 33.60 | 4.75 | 3 | 11.03 | 4.98 | 3 | 44.90 | 7.60 | 3 |
| L060 | 60.95 | 1.34 | 2 | 16.25 | 2.62 | 2 | 31.47 | 1.54 | 3 | 11.63 | 5.54 | 3 | 1.35 | 0.75 | 3 |
| L061 | 38.25 | 3.75 | 2 | 8.25 | 2.33 | 2 | 42.17 | 1.14 | 3 | 18.40 | 5.80 | 3 | 30.95 | 5.15 | 3 |
| L062 | 30.95 | 3.18 | 2 | 10.40 | 5.52 | 2 | 33.70 | 6.12 | 3 | 22.27 | 7.07 | 3 | 31.60 | 10.50 | 3 |
| L063 | 41.15 | 1.20 | 2 | 24.15 | 4.45 | 2 | 38.67 | 4.02 | 3 | 14.50 | 3.11 | 3 | 32.35 | 3.05 | 3 |
| L064 | 60.90 | 3.11 | 2 | 22.20 | 2.83 | 2 | 40.80 | 2.30 | 3 | 17.30 | 3.49 | 3 | 43.90 | 0.90 | 3 |
| L065 | 55.45 | 6.01 | 2 | 18.75 | 2.19 | 2 | 34.97 | 4.17 | 3 | 21.40 | 1.28 | 3 | 36.45 | 11.15 | 3 |
| L066 | 34.10 | 3.82 | 2 | 25.05 | 3.32 | 2 | 24.77 | 1.02 | 3 | 18.30 | 2.55 | 3 | 40.95 | 5.95 | 3 |
| L067 | 28.15 | 0.78 | 2 | 8.60 | 6.65 | 2 | 38.77 | 0.93 | 3 | 22.00 | 9.53 | 3 | 37.25 | 5.45 | 3 |
| L068 | 16.55 | 1.77 | 2 | 26.75 | 2.62 | 2 | 35.30 | 1.87 | 3 | 5.00 | 1.80 | 3 | 42.00 | 8.00 | 3 |
| L069 | 51.70 | 3.25 | 2 | 20.40 | 2.40 | 2 | 40.30 | 2.82 | 3 | 9.93 | 3.16 | 3 | 36.30 | 10.40 | 3 |
| No treatment | 0.15 | 0.07 | 2 | 0.15 | 0.07 | 2 | 1.27 | 1.36 | 3 | 0.17 | 0.09 | 3 | 0.20 | 0.00 | 3 |
| 1XNLS Cas9 | 0.15 | 0.21 | 2 | 0.00 | 0.00 | 2 | 0.00 | 0.00 | 3 | 0.03 | 0.05 | 3 | 0.10 | 0.10 | 3 |
| GFP | 0.40 | 0.28 | 2 | 0.00 | 0.00 | 2 | 0.33 | 0.12 | 3 | 0.23 | 0.05 | 3 | 0.25 | 0.05 | 3 |

### Example 26. Base Editing by Constructs with Various Linkers in Huh-7 Cells

Select base editor constructs derived from BC22 but with linkers from Table 60 substituted between the cytosine deaminase and Cas9 nickase were assayed for C to T base editing activity using Lipofectamine transfection of mRNA and gRNA. Tested base editor mRNAs include SEQ ID NOs: 19 and 347-357. Constructs were screened in a dilution series ranging from 150 ng to 1.17 ng of base editor mRNA in Huh-7 cells, co-delivered with a dilution series of SERPINA1 sgRNA (G000255) ranging from 20 to 0.15 nM and UGI mRNA (SEQ ID No: 25) at a dose of 25 to 0.20 ng in 100 uL of media. C to T base editing of each construct was compared to BC22 (SEQ ID No: 19).

### Example 26.1. Cell Preparation and Transfection

Huh-7 cells growing in Dulbecco's Modified Eagle's Medium (DMEM) supplemented with 10% Fetal Bovine Serum (FBS) in 96-well plates were transfected using Lipofectamine® RNAiMAX with an 8-point, two-fold dilution series starting with a maximum dose of 150 ng base editor mRNA, 25 ng UGI mRNA and 20 nM sgRNA in 100 uL media.

### Example 26.2. Evaluation of C to T Editing Purity by NGS

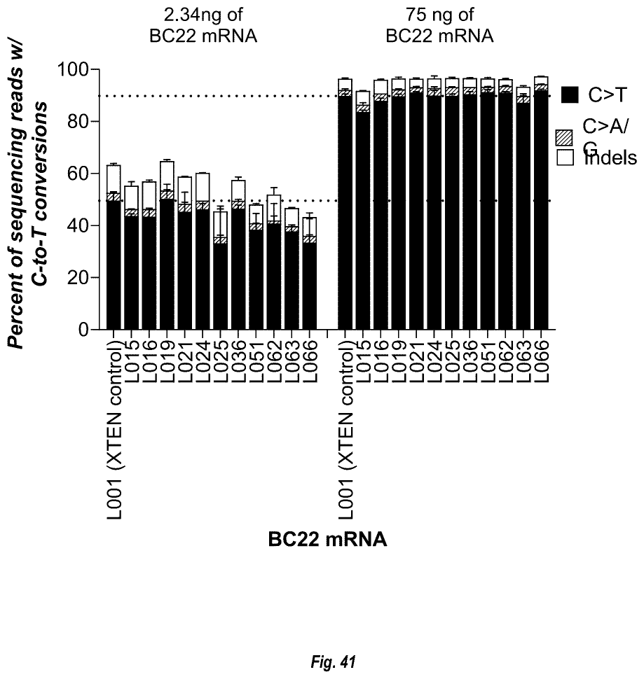

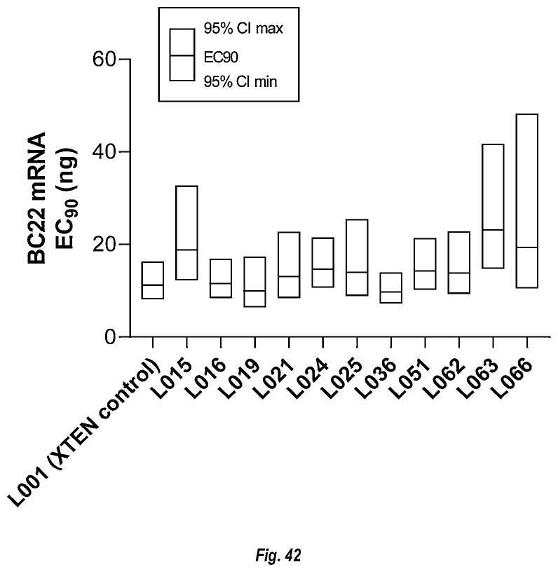

Seventy-two hours post transfection, Huh-7 cells were subjected to lysis, PCR amplification of the SERPINA1 locus and subsequent NGS analysis, as described in Example 1. Table 62 and FIG. 41 show SERPINA1 editing levels and the C to T editing purity in samples treated with either 2.3 or 75 ng of different base editor mRNAs and corresponding levels of diluted UGI mRNA and G000255 (SERPINA1). Mean percent C to T conversion for base editor mRNAs with different linkers are shown in Tables 63A-63B. Table 64 and FIG. 42 show EC90 (mass of base editor mRNA required to edit 90% of maximum C to T edits) ranges from 9.7 to 23.1 ng. All the tested base editor mRNAs similar levels of maximal editing at high doses

TABLE 62
Mean percent editing at SERPINA1 in Huh-7 Indels = insertions
or deletions.

| Base editor | | C to T (%) | | C to A/G (%) | | Indels (%) | |
|---|---|---|---|---|---|---|---|
| mRNA (ng) | Linker | Mean | SD | Mean | SD | Mean | SD |
| 2.3 | L001 | 49.6 | 3.0 | 3.0 | 0.4 | 10.7 | 0.6 |
| | (XTEN control) | | | | | | |
| | L015 | 43.7 | 0.8 | 2.7 | 0.1 | 9.0 | 1.6 |
| | L016 | 43.4 | 2.8 | 3.0 | 0.4 | 10.7 | 0.5 |
| | L019 | 50.2 | 5.7 | 3.3 | 0.4 | 11.3 | 0.6 |
| | L021 | 45.3 | 7.5 | 3.1 | 0.6 | 10.5 | 0.1 |
| | L024 | 46.3 | 2.2 | 3.3 | 0.0 | 10.7 | 0.1 |
| | L025 | 33.2 | 14.3 | 2.4 | 0.7 | 9.9 | 0.9 |
| | L036 | 46.5 | 1.4 | 3.0 | 0.6 | 8.0 | 1.2 |
| | L051 | 38.4 | 6.2 | 2.5 | 0.1 | 7.2 | 0.4 |
| | L062 | 40.8 | 7.7 | 1.2 | 1.7 | 10.0 | 2.6 |
| | L063 | 37.8 | 1.8 | 2.0 | 0.5 | 6.9 | 0.3 |
| | L066 | 33.4 | 9.2 | 2.7 | 0.4 | 7.2 | 1.6 |
| 75 | L001 | 89.8 | 0.8 | 2.3 | 0.4 | 4.4 | 0.2 |
| | (XTEN control) | | | | | | |
| | L015 | 83.6 | 0.8 | 2.9 | 0.8 | 5.3 | 0.1 |
| | L016 | 87.9 | 1.1 | 2.9 | 0.0 | 5.2 | 0.3 |
| | L019 | 89.6 | 0.8 | 2.7 | 0.2 | 4.3 | 0.5 |
| | L021 | 91.1 | 0.3 | 2.0 | 0.3 | 3.3 | 0.3 |
| | L024 | 89.9 | 2.1 | 3.0 | 0.5 | 3.8 | 0.9 |
| | L025 | 89.8 | 0.8 | 3.4 | 0.1 | 3.5 | 0.3 |
| | L036 | 90.4 | 1.0 | 2.8 | 0.0 | 3.4 | 0.1 |
| | L051 | 91.3 | 0.9 | 2.0 | 0.1 | 3.3 | 0.4 |
| | L062 | 91.1 | 0.4 | 2.6 | 0.2 | 2.7 | 0.2 |
| | L063 | 87.2 | 3.5 | 2.6 | 0.2 | 3.6 | 0.4 |
| | L066 | 91.9 | 0.4 | 2.4 | 0.1 | 3.1 | 0.1 |

TABLE 63A
Mean percent C to T editing at SERPINA1 in Huh-7 cells.

| | L001 | | | | | | | | | | | |
|---|---|---|---|---|---|---|---|---|---|---|---|---|
| Base editor | (XTEN control) | | L015 | | L016 | | L019 | | L021 | | L024 | |
| mRNA (ng) | Mean | SD | Mean | SD | Mean | SD | Mean | SD | Mean | SD | Mean | SD |
| 150.00 | 83.4 | 0.9 | 81.3 | 0.4 | 79.9 | 0.3 | 80.4 | 1.0 | 81.6 | 3.4 | 84.4 | — |
| 75.00 | 89.8 | 0.8 | 83.6 | 0.8 | 87.9 | 1.1 | 89.6 | 0.8 | 91.1 | 0.3 | 89.9 | 2.1 |
| 37.50 | 87.9 | 1.0 | 84.2 | 6.2 | 88.6 | 0.7 | 88.1 | 0.6 | 87.9 | 0.5 | 88.7 | 1.3 |
| 18.75 | 85.6 | 1.5 | 74.7 | 4.7 | 83.8 | 1.0 | 85.4 | 2.3 | 83.6 | 1.9 | 84.9 | 1.6 |
| 9.38 | 73.2 | 0.1 | 68.7 | 0.1 | 74.5 | 3.6 | 72.2 | 4.5 | 71.6 | 5.3 | 76.5 | 1.8 |
| 4.69 | 66.1 | 0.3 | 54.7 | 4.0 | 61.0 | 3.7 | 66.9 | 1.9 | 62.3 | 1.2 | 58.2 | 2.0 |
| 2.34 | 49.6 | 3.0 | 43.7 | 0.8 | 43.4 | 2.8 | 50.2 | 5.7 | 45.3 | 7.5 | 46.3 | 2.2 |
| 1.17 | 29.0 | 0.2 | 25.8 | 0.8 | 25.0 | 0.9 | 28.3 | 3.7 | 24.9 | 3.0 | 25.1 | 2.6 |

TABLE 63B
Mean percent C to T editing at SERPINA1 in Huh-7 cells.

| | L025 | | L036 | | L051 | | L062 | | L063 | | L066 | |
|---|---|---|---|---|---|---|---|---|---|---|---|---|
| Base editor mRNA (ng) | Mean | SD | Mean | SD | Mean | SD | Mean | SD | Mean | SD | Mean | SD |
| 150.00 | 81.5 | 3.4 | 82.2 | 3.7 | 84.0 | 0.5 | 84.3 | 1.6 | 78.7 | 2.4 | 78.8 | 3.5 |
| 75.00 | 89.8 | 0.8 | 90.4 | 1.0 | 91.3 | 0.9 | 91.1 | 0.4 | 87.2 | 3.5 | 91.9 | 0.4 |
| 37.50 | 85.4 | 4.0 | 90.0 | 2.2 | 89.6 | 2.6 | 89.1 | 1.8 | 81.7 | 1.6 | 89.5 | 0.7 |
| 18.75 | 83.1 | 1.0 | 87.0 | 1.3 | 84.1 | 2.9 | 86.2 | 1.0 | 74.9 | 1.8 | 84.8 | 0.6 |
| 9.38 | 71.7 | 0.3 | 78.9 | 0.5 | 76.8 | 0.7 | 73.9 | 7.4 | 67.2 | 2.5 | 67.9 | 1.0 |
| 4.69 | 59.3 | 2.1 | 68.3 | 2.2 | 58.7 | 5.1 | 62.7 | 4.1 | 48.6 | 2.0 | 55.7 | 8.5 |
| 2.34 | 33.2 | 14.3 | 46.5 | 1.4 | 38.4 | 6.2 | 40.8 | 7.7 | 37.8 | 1.8 | 33.4 | 9.2 |
| 1.17 | 23.1 | 0.7 | 30.8 | 1.4 | 25.1 | 1.1 | 26.8 | 1.2 | 23.5 | 3.6 | 28.1 | 2.8 |

TABLE 64
Mass of BC22 mRNAs that leads to 90% of the
maximum C to T editing at SERPINA1. The 95%
confidence interval for each EC90 value is also shown.

| | EC90 | 95% confidence interval | |
|---|---|---|---|
| Linker | (ng) | Max (ng) | Min (ng) |
| L001 (XTEN control) | 11.23 | 16.33 | 8.199 |
| L015 | 18.84 | 32.71 | 12.28 |
| L016 | 11.57 | 16.91 | 8.421 |
| L019 | 9.974 | 17.4 | 6.444 |
| L021 | 13.07 | 22.77 | 8.449 |
| L024 | 14.67 | 21.5 | 10.700 |
| L025 | 13.99 | 25.53 | 8.893 |
| L036 | 9.744 | 13.98 | 7.213 |
| L051 | 14.25 | 21.44 | 10.21 |
| L062 | 13.84 | 22.82 | 9.324 |
| L063 | 23.14 | 41.8 | 14.77 |
| L066 | 19.38 | 48.34 | 10.54 |

### Example 27. Base Editing by Constructs with Various Linkers in Primary Human Hepatocytes (PHH)

Select base editor constructs derived from BC22n but with linkers from Table 65 substituted between the cytosine deaminase and Cas9 nickase were assayed for C to T base editing activity in PHH. Constructs (SEQ ID NOs: 341-346) were screened in a 12-point dilution series of base editor mRNA in PHH cells, co-delivered with a fixed mass of ANAPC5 sgRNA (G019427) and UGI mRNA (SEQ ID NO: 34). The C to T editing efficiency of each tested based editor construct was compared to BC22n (SEQ ID NO: 1).

TABLE 65
Additional linker peptides.

| Linker ID | Linker amino acid sequence |
|---|---|
| L001 | SGSETPGTSESATPES (SEQ ID NO: 46) |
| (XTEN | |
| control) | |
| L070 | GTKDSTKDIPETPSKD (SEQ ID NO: 268) |
| L071 | GRDVRQPEVKEEKPES (SEQ ID NO: 269) |
| L072 | EGKSSGSGSESKSTAG (SEQ ID NO: 270) |
| L073 | TPGSPAGSPTSTEEGT (SEQ ID NO: 271) |
| L074 | GSEPATSGSETPGTST (SEQ ID NO: 272) |
| L019 | EAAAKEAAAKEAAAK (SEQ ID NO: 51) |

### Example 27.1. Cell Preparation and Transfection

PHH cells are thawed and recovered in CHRMs media (Gibco, cat #CM7000). They are then resuspended in Primary Hepatocyte Plating Media (Consisting of William's E media (Gibco, Cat #A1217601) and primary hepatocyte plating supplements (Gibco, Cat #CM3000))) to be plated in collagen-coated 96-well plates at a density of 33,000 cells/well for twenty-four hours. Post twenty-four hours, cells are washed, and fresh primary hepatocyte maintenance media is added on cells. Cells are then transfected simultaneously with separate lipoplexes formed individually with either UGI mRNA (SEQ ID No: 34), ANAPC5 sgRNA G019427 or base editor mRNA.

Lipofection reagent was prepared as mixture of lipids at a ratio of 50/9/38/3 Lipid A, DSPC, cholesterol and PEG2k-DMG as described in Example 1. Lipofection reagent was combined by bulk mixing with separately with each RNA species: base editor mRNA, UGI mRNA or gRNA G019427. The materials were combined at a lipid amine to RNA phosphate (N:P) molar ratio of about 6. The resulting bulk-mixed lipoplex material (lipid kits) was pre-incubated with 10% FBS (Gibco; A3160501) in Primary Hepatocyte Maintenance Media (Consisting of William's E media (Gibco, Cat #A1217601) and primary hepatocyte maintenance supplements (Gibco, Cat #CM4000)), for 15 min before addition to hepatocytes.

Each well received three components in a final volume of 100 uL: base editor mRNAs ranging from 400 ng to 0 ng of mRNAs, 30 ng of UGI mRNA and 5 pmols of G019427 (ANAPC5).

### Example 27.2 Evaluation of C to T Editing Purity by Next Generation Sequencing (NGS)

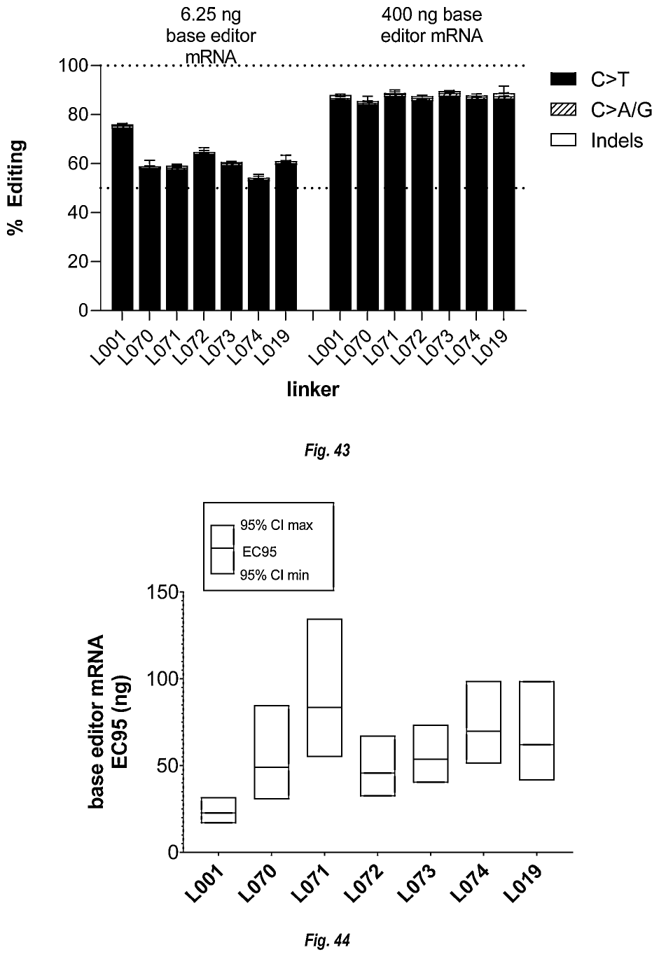

Seventy-two hours post transfection, PHH cells were subjected to lysis, PCR amplification of the ANAPC5 locus and subsequent NGS analysis, as described in Example 1. Table 66 and FIG. 43 show ANAPC5 total editing levels and the C to T editing purity in samples treated with either 400 or 6.25 ng of different base editor mRNAs in addition to 30 ng of UGI mRNA (SEQ ID No: 34) and 5 pmols of G019427 (ANAPC5).

C to T base editing for a range of base editor mRNAs doses is shown in Table 67. The EC95 (mass of BC22n mRNA required to edit 95% of maximum C to T edits) was calculated as shown in Table 68 and FIG. 44. All of the base editor mRNAs were able to achieve the similar levels of maximal editing at high doses.

TABLE 66
Mean percent editing at ANAPC5 in PHH cells using base
editing constructs designed with various
peptide linkers. Indels = insertions or deletions.

| Base editor | | | | | | | |
|---|---|---|---|---|---|---|---|
| mRNA | | C TO T (%) | | C to A/G (%) | | Indels (%) | |
| (ng) | Linker | Mean | SD | Mean | SD | Mean | SD |
| 6.25 | L001 | 74.9 | 1.6 | 0.6 | 0.1 | 0.6 | 0.2 |
| | (XTEN control) | | | | | | |
| | L070 | 58.0 | 3.4 | 0.5 | 0.0 | 0.5 | 0.1 |
| | L071 | 57.8 | 2.0 | 0.6 | 0.1 | 0.9 | 0.2 |
| | L072 | 63.7 | 2.8 | 0.4 | 0.1 | 0.7 | 0.6 |
| | L073 | 59.7 | 1.3 | 0.5 | 0.1 | 0.6 | 0.1 |
| | L074 | 53.1 | 2.5 | 0.5 | 0.3 | 0.7 | 0.6 |
| | L019 | 59.9 | 3.5 | 0.4 | 0.1 | 0.8 | 0.2 |
| 400 | L001 | 86.3 | 0.1 | 0.6 | 0.4 | 1.2 | 0.4 |
| | (XTEN control) | | | | | | |
| | L070 | 84.3 | 3.3 | 0.6 | 0.2 | 0.7 | 0.1 |
| | L071 | 87.7 | 2.4 | 0.7 | 0.2 | 0.5 | 0.4 |
| | L072 | 86.0 | 0.1 | 0.9 | 0.4 | 0.7 | 0.4 |
| | L073 | 87.9 | 1.8 | 0.9 | 0.0 | 0.8 | 0.3 |
| | L074 | 86.6 | 1.8 | 0.6 | 0.1 | 0.7 | 0.0 |
| | L019 | 86.7 | 4.9 | 0.9 | 0.0 | 1.2 | 0.1 |

TABLE 67
Mean percent C to T editing at the ANAPC5 locus in PHH for base editing constructs designed with various peptide linkers.

| | L001 | | L070 | | L071 | | L072 | | L073 | | L074 | | L019 | |
|---|---|---|---|---|---|---|---|---|---|---|---|---|---|---|
| mRNA (ng) | Mean | SD | Mean | SD | Mean | SD | Mean | SD | Mean | SD | Mean | SD | Mean | SD |
| 400.00 | 86.3 | 0.1 | 84.3 | 3.3 | 87.7 | 2.4 | 86.0 | 0.1 | 87.9 | 1.8 | 86.6 | 1.8 | 86.7 | 4.9 |
| 200.00 | 87.8 | 2.4 | 83.9 | 1.1 | 88.1 | 0.5 | 86.9 | 5.4 | 87.6 | 1.8 | 86.6 | 2.5 | 87.4 | 3.2 |
| 100.00 | 91.1 | 0.2 | 84.7 | 2.3 | 84.6 | 4.5 | 87.0 | 2.5 | 83.6 | 0.1 | 85.1 | 4.7 | 85.9 | 2.5 |
| 50.00 | 89.0 | 1.8 | 78.0 | 5.4 | 82.4 | 5.0 | 85.8 | 0.6 | 83.9 | 1.8 | 84.3 | 0.8 | 85.0 | 3.7 |
| 25.00 | 86.9 | 1.8 | 79.1 | 0.2 | 78.6 | 4.6 | 81.3 | 3.3 | 79.4 | 2.3 | 79.4 | 0.5 | 82.7 | 1.1 |
| 12.50 | 83.2 | 0.8 | 71.3 | 2.5 | 69.3 | 6.0 | 71.8 | 1.6 | 72.6 | 4.4 | 69.2 | 0.9 | 72.8 | 2.5 |
| 6.25 | 74.9 | 1.6 | 58.0 | 3.4 | 57.8 | 2.0 | 63.7 | 2.8 | 59.7 | 1.3 | 53.1 | 2.5 | 59.9 | 3.5 |
| 3.13 | 64.3 | 2.9 | 40.5 | 3.0 | 42.8 | 2.9 | 47.8 | 1.4 | 42.4 | 0.6 | 37.7 | 2.1 | 45.6 | 1.6 |
| 1.56 | 50.8 | 2.7 | 24.6 | 6.6 | 30.8 | 0.2 | 28.3 | 0.7 | 27.8 | 0.7 | 27.1 | 0.7 | 31.6 | 1.1 |
| 0.78 | 35.7 | 2.3 | 18.8 | 0.0 | 20.1 | 0.0 | 20.8 | 0.7 | 17.4 | 0.8 | 16.0 | 0.8 | 22.8 | 3.7 |
| 0.39 | 23.6 | 3.9 | 12.1 | 1.7 | 12.1 | 0.7 | 13.2 | 0.6 | 12.5 | 1.1 | 10.0 | 0.6 | 14.6 | 0.8 |
| 0.00 | 0.4 | 0.2 | 0.6 | 0.1 | 0.5 | 0.0 | 0.6 | 0.2 | 0.4 | 0.1 | 0.4 | 0.3 | 0.6 | 0.1 |

TABLE 68
Mass of base editor mRNAs that leads to 95% of the maximum C to T
editing. The 95% confidence interval for each EC95 value is also shown.

| | | 95% confidence interval | |
|---|---|---|---|
| Linker | EC95 (ng) | Max (ng) | Min (ng) |
| L001 (XTEN control) | 22.73 | 31.91 | 16.75 |
| L070 | 48.99 | 84.83 | 30.52 |
| L071 | 83.46 | 134.60 | 54.97 |
| L072 | 45.72 | 67.33 | 32.28 |
| L073 | 53.62 | 73.54 | 40.17 |
| L074 | 69.89 | 98.83 | 51.13 |
| L019 | 62.06 | 98.67 | 41.39 |

### Example 28. Base Editing by Constructs with Various Linkers in T Cells

Select base editor constructs derived from BC22n but with linkers from Table 65 substituted between the cytosine deaminase and Cas9 nickase were assayed for C to T base editing activity. Constructs (SEQ ID Nos: 341-346) were screened in a 12-point dilution series of base editor mRNA in healthy human T cells, co-delivered with a fixed amount of TRAC sgRNA (G016017) and of UGI mRNA (SEQ ID NO: 34). The C to T editing efficiency of each base editor mRNA construct was compared to BC22n (SEQ ID NO: 1).

### Example 28.1. T Cell Preparation

Healthy human donor apheresis was obtained commercially (Hemacare), and cells were washed, re-suspended in CliniMACS® PBS/EDTA buffer (Miltenyi Biotec Cat. 130-070-525) and processed in a MultiMACS™ Cell 24 Separator Plus device (Miltenyi Biotec). T cells were isolated via positive selection using a Straight from Leukopak® CD4/CD8 MicroBead kit, human (Miltenyi Biotec Cat. 130-122-352). T cells were aliquoted and cryopreserved for future use in Cryostor® CS10 (StemCell Technologies Cat. 07930).

Upon thaw, T cells were plated at a density of 1.0×10\hat{ }6 cells/mL in T cell growth media (TCGM) composed of CTS OpTmizer T Cell Expansion SFM and T Cell Expansion Supplement (ThermoFisher Cat. A1048501), 5% human AB serum (GeminiBio, Cat. 100-512) 1× Penicillin-Streptomycin, 1× Glutamax, 10 mM HEPES, 200 U/mL recombinant human interleukin-2 (Peprotech, Cat. 200-02), 5 ng/ml recombinant human interleukin 7 (Peprotech, Cat. 200-07), and 5 ng/ml recombinant human interleukin 15 (Peprotech, Cat. 200-15). T cells were rested in this media for 24 hours, at which time they were activated with T Cell TransAct™, human reagent (Miltenyi, Cat. 130-111-160) added at a 1:100 ratio by volume. T cells were activated for 48 hours prior to use in screening experiments.

### Example 28.2. T Cell Electroporation and Expansion

A sgRNA targeting TRAC (G016017) was denatured for 2 minutes at 95° C., incubated at room temperature for 5 minutes and stored on ice. 48 hours post-activation, T cells were harvested, centrifuged at 500 g for 5 min, and resuspended at a concentration of 12.5×10\hat{ }6 T cells/mL in P3 electroporation buffer (Lonza). For each well to be electroporated, 1×10\hat{ }5 T cells were mixed with a decreasing amount of base editor mRNAs ranging from 400 ng to 0 ng, 200 ng of UGI mRNA and 40 pmols of G016017 in a final volume of 20 uL of P3 electroporation buffer.

The resulting mix was transferred in duplicate to 96-well Nucleofector™ plates (Lonza) and electroporated using the manufacturer's pulse code. Immediately after electroporation, T cells received 80 μL of TCGM and plates were incubated at 37° C. for 15 minutes. After incubation, 80 μL were transferred to new flat-bottom 96-well plates containing 80 μL of TCGM. T cells were incubated at 37° C. for 4 days, at which time they were mixed, split 1:4 with fresh TCGM, and incubated at for 3 additional days prior to phenotypic assessments by flow cytometry.

### Example 28.3. Evaluation of C to T Editing Purity by Next Generation Sequencing (NGS)

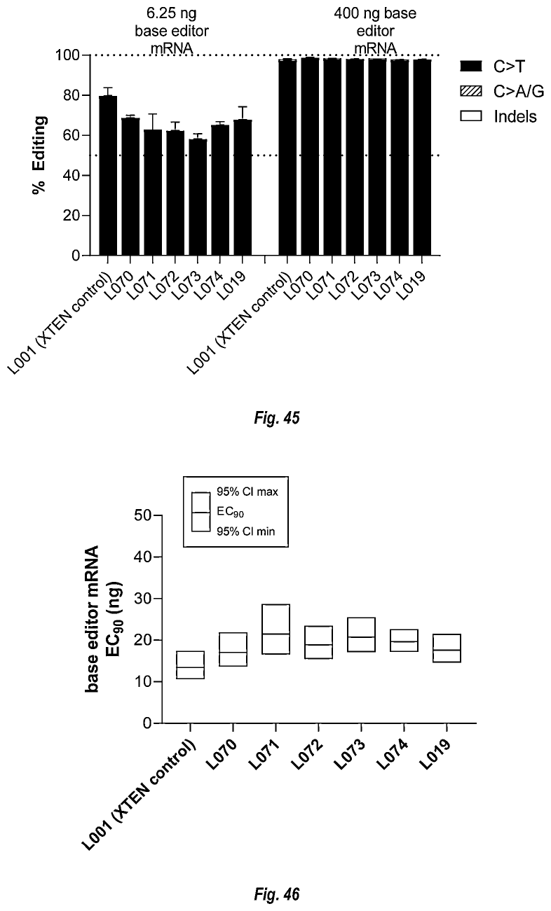

Four days post electroporation, T cells were subjected to lysis, PCR amplification of the TRAC locus and subsequent NGS analysis, as described in Example 1. Table 69 and FIG. 45 show TRAC editing levels and the C to T editing purity in samples treated with either 400 or 6.25 ng of different base editor mRNAs in addition to 200 ng of UGI mRNA (SEQ ID No: 34) and 40 pmols of G016017 (TRAC).

None of the linkers tested between the N-terminal cytosine deaminase and C-terminal Cas9 nickase limit the efficiency of cytosine base editing or impacts the levels of C to T editing purity.

TABLE 69
Mean percent editing at the TRAC locus in T cells treated with
base editor constructs designed with various linker peptides.

| base editor | | C to T (%) | | C to A/G (%) | | Indels (%) | |
|---|---|---|---|---|---|---|---|
| mRNA (ng) | Linker | Mean | SD | Mean | SD | Mean | SD |
| 6.25 | L001 | 78.9 | 4.9 | 0.3 | 0.1 | 0.3 | 0.3 |
| | (XTEN control) | | | | | | |
| | L070 | 68.2 | 1.8 | 0.3 | 0.2 | 0.2 | 0.1 |
| | L071 | 62.3 | 8.5 | 0.2 | 0.1 | 0.4 | 0.0 |
| | L072 | 62.0 | 4.7 | 0.1 | 0.2 | 0.2 | 0.1 |
| | L073 | 57.4 | 3.4 | 0.4 | 0.4 | 0.3 | 0.1 |
| | L074 | 64.8 | 2.1 | 0.1 | 0.0 | 0.3 | 0.0 |
| | L019 | 67.3 | 7.0 | 0.3 | 0.0 | 0.2 | 0.0 |
| 400 | L001 | 97.0 | 0.9 | 0.3 | 0.1 | 0.7 | 0.3 |
| | (XTEN control) | | | | | | |
| | L070 | 98.1 | 0.1 | 0.2 | 0.1 | 0.4 | 0.2 |
| | L071 | 97.7 | 0.5 | 0.3 | 0.1 | 0.5 | 0.1 |
| | L072 | 97.6 | 0.6 | 0.2 | 0.0 | 0.3 | 0.0 |
| | L073 | 97.5 | 0.7 | 0.4 | 0.0 | 0.6 | 0.0 |
| | L074 | 96.9 | 0.5 | 0.4 | 0.3 | 0.6 | 0.1 |
| | L019 | 97.4 | 0.3 | 0.3 | 0.2 | 0.2 | 0.2 |

Indels = insertions or deletions.

### Example 28.4. Evaluation of Receptor Knockout by Flow Cytometry

Seven days post electroporation, T cells were assayed by flow cytometry to evaluate loss of CD3 expression. T cells were incubated with a fixable viability dye (Beckman Coulter, Cat. C36628) and an antibody cocktail targeting the following molecules: CD3 (Biolegend, Cat. 317336), CD4 (Biolegend, Cat. 317434) and CD8 (Biolegend, Cat. 301046). Cells were subsequently washed, analyzed on a Cytoflex LX instrument (Beckman Coulter) using the FlowJo software package. T cells were gated on size, viability and CD8 positivity before expression of any markers was determined. The resulting data was plotted on GraphPad Prism v. 9.0.2 and analyzed using a variable slope (four parameter) non-linear regression.

As shown in Table 70, treatment with >100 ng of any of base editor mRNA tested resulted in >95% of CD8+ T cells lacking expression of CD3. The EC90s of the base editor mRNAs tested (i.e., mass of mRNA that leads to 90% of CD8+ T cells lacking CD3) with 95% confidence interval of each non-linear regression are shown in Table 71 and FIG. 46.

TABLE 70
Mean percentages of CD8+ T cells that are negative for CD3 surface expression following treatment with base editor
constructs designed with various linker peptides.

| Base editor | L001 (XTEN control) | | L070 | | L071 | | L072 | | L073 | | L074 | | L019 | |
|---|---|---|---|---|---|---|---|---|---|---|---|---|---|---|
| mRNA (ng) | Mean | SD | Mean | SD | Mean | SD | Mean | SD | Mean | SD | Mean | SD | Mean | SD |
| 400.00 | 96.5 | 0.3 | 97.2 | 0.5 | 96.1 | 0.3 | 96.1 | 0.4 | 96.2 | 0.2 | 96.2 | 0.4 | 95.6 | 0.4 |
| 200.00 | 96.8 | 0.8 | 96.4 | 1.8 | 96.7 | 0.8 | 96.8 | 0.9 | 96.1 | 1.0 | 95.7 | 0.9 | 96.2 | 1.0 |
| 100.00 | 97.1 | 0.1 | 96.2 | 1.6 | 97.0 | 0.2 | 95.0 | 1.4 | 96.0 | 0.7 | 96.4 | 0.7 | 95.3 | 0.3 |
| 50.00 | 96.3 | 0.7 | 96.0 | 0.6 | 95.2 | 1.2 | 95.2 | 1.6 | 94.6 | 2.3 | 94.5 | 1.2 | 95.8 | 1.3 |
| 25.00 | 94.9 | 1.5 | 92.8 | 3.4 | 92.1 | 3.7 | 90.7 | 2.4 | 90.7 | 0.3 | 91.5 | 1.2 | 91.7 | 1.1 |
| 12.50 | 91.5 | 1.6 | 85.7 | 5.1 | 82.8 | 5.9 | 83.6 | 4.2 | 82.5 | 2.3 | 79.9 | 0.6 | 85.0 | 1.8 |
| 6.25 | 76.1 | 7.9 | 69.0 | 5.4 | 61.8 | 8.6 | 63.8 | 3.6 | 61.8 | 3.1 | 65.7 | 4.1 | 67.1 | 5.6 |
| 3.13 | 56.5 | 6.2 | 49.0 | 5.5 | 42.6 | 6.3 | 42.2 | 5.7 | 43.0 | 6.7 | 43.7 | 3.2 | 47.7 | 5.5 |
| 1.56 | 37.8 | 2.2 | 29.0 | 4.9 | 26.9 | 2.8 | 24.1 | 5.4 | 24.4 | 3.2 | 25.1 | 2.7 | 29.0 | 3.5 |
| 0.78 | 23.5 | 3.7 | 17.6 | 3.7 | 14.0 | 0.1 | 13.8 | 2.1 | 14.0 | 3.1 | 13.5 | 1.8 | 16.3 | 2.1 |
| 0.39 | 13.2 | 2.9 | 9.0 | 2.3 | 7.6 | 0.8 | 7.7 | 0.5 | 7.0 | 0.3 | 6.5 | 0.7 | 9.0 | 0.9 |
| 0.00 | 1.9 | 0.5 | 1.5 | 0.1 | 1.4 | 0.7 | 2.1 | 1.3 | 1.3 | 0.4 | 1.6 | 0.7 | 2.1 | 0.5 |

TABLE 71
Mass of base editor mRNAs that leads to 90% of the
maximum knockdown of CD3. The 95%
confidence interval for each EC90 value is also shown.

| | EC90 | 95% confidence interval | |
|---|---|---|---|
| Linker | (ng) | Min (ng) | Max (ng) |
| L001 (XTEN control) | 13.4 | 10.6 | 17.5 |
| L070 | 17.1 | 13.6 | 21.9 |
| L071 | 21.5 | 16.6 | 28.7 |
| L072 | 18.9 | 15.5 | 23.5 |
| L073 | 20.7 | 17.1 | 25.5 |
| L074 | 19.7 | 17.2 | 22.6 |
| L019 | 17.6 | 14.6 | 21.5 |

### Example 29. Multiplex Base Editing by Constructs with Various Linker in T Cells

Select base editor constructs derived from BC22n but with linkers from Table 65 substituted between the cytosine deaminase and Cas9 nickase were assayed for multiple simultaneous base editing efficacy via protein knockdown. Constructs were screened in a 12-point dilution series of base editor mRNA in healthy human T cells, co-delivered with a fixed mass of UGI mRNA (SEQ ID NO: 34) and 4 distinct 91-mer sgRNAs. The potency of each mRNA construct was compared to BC22n mRNA (SEQ ID NO: 1) to evaluate the effects of each linker in terms of phenotypic receptor knockout.

### Example 29.1. T Cell Preparation

Healthy human donor apheresis was obtained commercially (Hemacare), and cells were washed, re-suspended in CliniMACS® PBS/EDTA buffer (Miltenyi Biotec Cat. 130-070-525) and processed in a MultiMACS™ Cell 24 Separator Plus device (Miltenyi Biotec). T cells were isolated via positive selection using a Straight from Leukopak® CD4/CD8 MicroBead kit, human (Miltenyi Biotec Cat. 130-122-352). T cells were aliquoted and cryopreserved for future use in Cryostor® CS10 (StemCell Technologies Cat. 07930).

Upon thaw, T cells were plated at a density of 1.0×10\hat{ }6 cells/mL in T cell growth media (TCGM) composed of CTS OpTmizer T Cell Expansion SFM and T Cell Expansion Supplement (ThermoFisher Cat. A1048501), 5% human AB serum (GeminiBio, Cat. 100-512) 1× Penicillin-Streptomycin, 1× Glutamax, 10 mM HEPES, 200 U/mL recombinant human interleukin-2 (Peprotech, Cat. 200-02), 5 ng/ml recombinant human interleukin 7 (Peprotech, Cat. 200-07), and 5 ng/ml recombinant human interleukin 15 (Peprotech, Cat. 200-15). T cells were rested in this media for 24 hours, at which time they were activated with T Cell TransAct™, human reagent (Miltenyi, Cat. 130-111-160) added at a 1:100 ratio by volume. T cells were activated for 48 hours prior to use in screening experiments.

### Example 29.2. T Cell Electroporation and Expansion

Four 91-mer sgRNAs targeting TRAC (G023520), TRBC1/2 (G023524), CIITA (G023521) and HLA-A (G023523) were denatured for 2 minutes at 95° C., incubated at room temperature for 5 minutes and stored on ice. 48 hours post-activation, T cells were harvested, centrifuged at 500 g for 5 min, and resuspended at a concentration of 12.5×10\hat{ }6 T cells/mL in P3 electroporation buffer (Lonza). For each well to be electroporated, 1×10\hat{ }5 T cells were mixed with a dilution series of base editor mRNAs from 400 ng to 0 ng (SEQ ID NOs: 1 and 321-326), 200 ng of UGI mRNA (SEQ ID NO: 34), 3.6 pmols of G023520 (TRAC), 6.96 pmols of G023524 (TRBC1/2), 17.12 pmols of G023521 (CIITA) and 52.24 pmols of G023523 (HLA-A) in a final volume of 20 uL of P3 electroporation buffer.

The resulting mix was transferred in duplicate to 96-well Nucleofector™ plates (Lonza) and electroporated using the manufacturer's pulse code. Immediately after electroporation, T cells received 80 μL of TCGM and plates were incubated at 37° C. for 15 minutes. After incubation, 80 μL were transferred to new flat-bottom 96-well plates containing 80 μL of TCGM. T cells were incubated at 37° C. for 4 days, at which time they split 1:4 with fresh TCGM, and incubated at for 3 additional days prior to phenotypic assessments by flow cytometry.

### Example 29.3. Evaluation of Receptor Knockout by Flow Cytometry

Seven days post electroporation, T cells were assayed by flow cytometry to evaluate loss of CD3, HLA-A3 and/or HLA-DR, DP, DQ. T cells were incubated with a fixable viability dye (Beckman Coulter, Cat. C36628) and an antibody cocktail targeting the following molecules: CD3 (Biolegend, Cat. 317336), CD4 (Biolegend, Cat. 317434), CD8 (Biolegend, Cat. 301046), HLA-A3 (ThermoFisher, Cat. 12-5754-42) and HLA-DP, DQ, DR (Biolegend, Cat. 361714). Cells were subsequently washed, analyzed on a Cytoflex LX instrument (Beckman Coulter) using the FlowJo software package. T cells were gated on size, viability and CD8 positivity before expression of any markers was determined. The resulting data was plotted on GraphPad Prism v. 9.0.2 and analyzed using a variable slope (four parameter) non-linear regression.

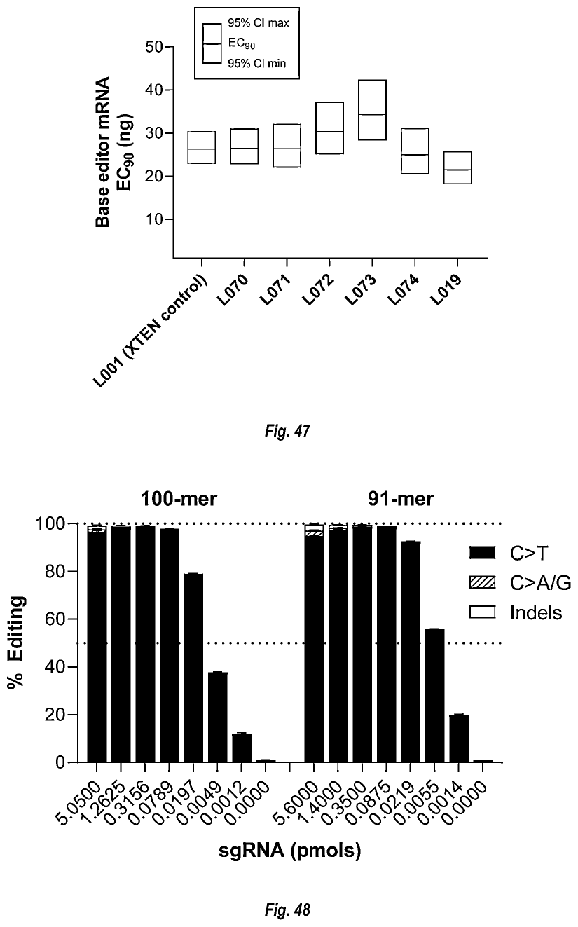

As shown in Table 72 treatment with >100 ng of any of BC22n mRNA tested resulted in >97% of CD8+ T cells lacking expression of CD3, HLA-A3 and HLA-DR, DP, DQ. The EC90s of the base editor mRNAs tested (i.e., mass of mRNA that leads to 90% of CD8+ T cells lacking CD3) with 95% confidence interval of each non-linear regression are shown in Table 73 and FIG. 47.

TABLE 72
Mean percentages of CD8+ T cells that are negative for CD3, HLA-A3 and HLA-DR, DP, DQ surface expression following
treatment with base editor mRNAs.

| | L001 (XTEN control) | | L070 | | L071 | | L072 | | L073 | | L074 | | L019 | |
|---|---|---|---|---|---|---|---|---|---|---|---|---|---|---|
| mRNA (ng) | Mean | SD | Mean | SD | Mean | SD | Mean | SD | Mean | SD | Mean | SD | Mean | SD |
| 400.00 | 97.4 | 0.5 | 98.3 | ND | 98.4 | 0.3 | 98.7 | 0.1 | 97.8 | 0.7 | 98.1 | 0.5 | 98.6 | 0.4 |
| 200.00 | 98.4 | 0.1 | 98.6 | 0.2 | 98.2 | 0.2 | 98.3 | 0.4 | 98.1 | 0.7 | 98.7 | 0.1 | 98.9 | 0.1 |
| 100.00 | 97.1 | 0.8 | 97.5 | 1.1 | 97.0 | 2.1 | 97.7 | 0.5 | 97.4 | 0.5 | 98.5 | 0.5 | 98.7 | 0.1 |
| 50.00 | 95.7 | 0.1 | 96.6 | 0.2 | 95.4 | 0.9 | 95.6 | 0.8 | 95.1 | 2.8 | 97.4 | 0.7 | 97.1 | 0.3 |
| 25.00 | 90.8 | 0.1 | 88.5 | 4.0 | 89.7 | 3.3 | 89.1 | 2.8 | 87.6 | 3.2 | 91.5 | 2.6 | 93.6 | 1.9 |
| 12.50 | 75.5 | 3.2 | 77.8 | 3.7 | 75.6 | 4.8 | 71.6 | 6.6 | 69.1 | 3.2 | 77.9 | 4.7 | 82.6 | 3.3 |
| 6.25 | 54.9 | 4.2 | 54.0 | 1.1 | 54.8 | 6.2 | 53.3 | 5.5 | 46.2 | 5.9 | 55.6 | 7.8 | 60.0 | 5.1 |
| 3.13 | 32.9 | 2.1 | 32.7 | 1.9 | 31.5 | 3.2 | 30.5 | 2.6 | 27.6 | 2.5 | 32.9 | 3.6 | 35.7 | 2.5 |
| 1.56 | 17.1 | 0.5 | 16.6 | 1.6 | 16.3 | 2.4 | 16.1 | 1.6 | 15.5 | 3.1 | 18.4 | 2.5 | 21.0 | 2.1 |
| 0.78 | 10.0 | 1.3 | 9.4 | 1.2 | 9.5 | 0.3 | 7.5 | 0.4 | 7.3 | 0.5 | 9.6 | 2.9 | 11.7 | 0.3 |
| 0.39 | 5.1 | 0.4 | 4.5 | 0.6 | 4.7 | 0.4 | 4.5 | 0.8 | 4.4 | 1.2 | 5.2 | 0.0 | 6.8 | 1.4 |
| 0.00 | 0.2 | 0.1 | 0.1 | 0.1 | 0.2 | 0.1 | 0.1 | 0.1 | 0.3 | 0.2 | 0.2 | 0.2 | 0.2 | 0.1 |

TABLE 73
Mass of base editor mRNAs that leads to 90% of the maximum knockdown
of CD3, HLA-A3 and HLA-DR, DP, DQ (EC90). The 95% confidence interval for each
EC90 value is also shown.

| | EC90 | 95% confidence interval | |
|---|---|---|---|
| Linker | (ng) | Min (ng) | Max (ng) |
| L001 (XTEN control) | 26.3 | 22.9 | 30.4 |
| L070 | 26.5 | 22.8 | 31.1 |
| L071 | 26.4 | 22.0 | 32.1 |
| L072 | 30.3 | 25.1 | 37.3 |
| L073 | 34.3 | 28.3 | 42.4 |
| L074 | 25.0 | 20.4 | 31.2 |
| L019 | 4.709 | 4.314 | 5.130 |

### Example 30 Editing Human T Cells with BC22n, UGI and 91-Mer sgRNAs

The base editing efficacy of 91-mer sgRNAs as assessed by NGS and/or receptor knockout was compared to that of 100-mer sgRNA controls with the same guides sequences.

The tested 91-mer sgRNAs include a 20 nucleotide guide sequence (as represented by N) and a guide scaffold as follows: mN*mN*mN*NNNNNNNNNNNNNNNNNGUUUUAGAmGmCmUmAmGmAmAmAmU mAmGmCAAGUUAAAAUAAGGCUAGUCCGUUAUCACGAAAGGGCACCGAGUCG GmUmGmC*mU (SEQ ID NO: 520), where A, C, G, U, and N are adenine, cytosine, guanine, uracil, and any ribonucleotide, respectively, unless otherwise indicated. An m is indicative of a 2′O-methyl modification, and an * is indicative of a phosphorothioate linkage between the nucleotides. Unmodified and modified versions of the guides are provided in Table 5C (Sequence Table).

### Example 30.1. T Cell Preparation

Healthy human donor apheresis was obtained commercially (Hemacare), and cells were washed, re-suspended in CliniMACS® PBS/EDTA buffer (Miltenyi Biotec Cat. 130-070-525) and processed in a MultiMACS™ Cell 24 Separator Plus device (Miltenyi Biotec). T cells were isolated via positive selection using a Straight from Leukopak® CD4/CD8 MicroBead kit, human (Miltenyi Biotec Cat. 130-122-352). T cells were aliquoted and cryopreserved for future use in Cryostor® CS10 (StemCell Technologies Cat. 07930).

Upon thaw, T cells were plated at a density of 1.0×10\hat{ }6 cells/mL in T cell growth media (TCGM) composed of CTS OpTmizer T Cell Expansion SFM and T Cell Expansion Supplement (ThermoFisher Cat. A1048501), 5% human AB serum (GeminiBio, Cat. 100-512) 1× Penicillin-Streptomycin, 1× Glutamax, 10 mM HEPES, 200 U/mL recombinant human interleukin-2 (Peprotech, Cat. 200-02), 5 ng/ml recombinant human interleukin 7 (Peprotech, Cat. 200-07), and 5 ng/ml recombinant human interleukin 15 (Peprotech, Cat. 200-15). T cells were rested in this media for 24 hours, at which time they were activated with T Cell TransAct™, human reagent (Miltenyi, Cat. 130-111-160) added at a 1:100 ratio by volume. T cells were activated for 48 hours prior to LNP treatments.

### Example 30.2. T Cell LNP Treatment and Expansion

Forty-eight hours post-activation, T cells were harvested, centrifuged at 500 g for 5 min, and resuspended at a concentration of 1×10\hat{ }6 T cells/mL in T cell plating media (TCPM): a serum-free version of TCGM containing 400 U/mL recombinant human interleukin-2 (Peprotech, Cat. 200-02), 10 ng/ml recombinant human interleukin 7 (Peprotech, Cat. 200-07), and 10 ng/ml recombinant human interleukin 15 (Peprotech, Cat. 200-15). 50 μL of T cells in TCPM (5×10\hat{ }4 T cells) were added per well to be treated in flat-bottom 96-well plates.

LNPs were prepared as described in Example 1 at a ratio of 35/47.5/15/2.5 (Lipid A/cholesterol/DSPC/PEG2k-DMG). The LNPs were formulated with a lipid amine to RNA phosphate (N:P) molar ratio of about 6. LNPs encapsulated a single RNA species, either a sgRNA as described in Table 74, BC22n mRNA (SEQ ID No: 1), or UGI mRNA (SEQ ID No. 34).

TABLE 74
100-mer and 91-mer sgRNAs.

| Gene target | 100-mer | 91-mer |
|---|---|---|
| TRAC | G016017 | G023520 |
| TRBC1/2 | G016200 | G023524 |
| CIITA | G016086 | G023521 |
| B2M | G015991 | G023519 |
| CD38 | G019771 | G023522 |
| HLA-A | G021209 | G023523 |

Prior to T cell treatment, LNPs encapsulating a sgRNA were diluted to 6.64 μg/mL in T cell treatment media (TCTM): a version of TCGM containing 20 ug/mL rhApoE3 in the absence of interleukins 2, 5 or 7. These LNPs were incubated at 37° C. for 15 minutes and serially diluted 1:4 using TCTM, which resulted in an 8-point dilution series ranging from 6.64 μg/mL to zero. Similarly, single-cargo LNPs with BC22n mRNA (SEQ ID NO: 1) or UGI mRNA (SEQ ID NO: 34) were diluted in TCTM to 3.32 and 1.67 μg/mL, respectively, incubated at 37° C. for 15 minutes, and mixed 1:1 by volume with sgRNA LNPs serially diluted in the previous step. Last, 50 μL from the resulting mix was added to T cells in 96-well plates at a 1:1 ratio by volume. T cells were incubated at 37° C. for 24 hours, at which time they were harvested, centrifuged at 500 g for 5 min, resuspended in 200 μL of TCGM and returned to the incubator.

### Example 30.3. Evaluation of Editing Outcomes by Next Generation Sequencing (NGS)

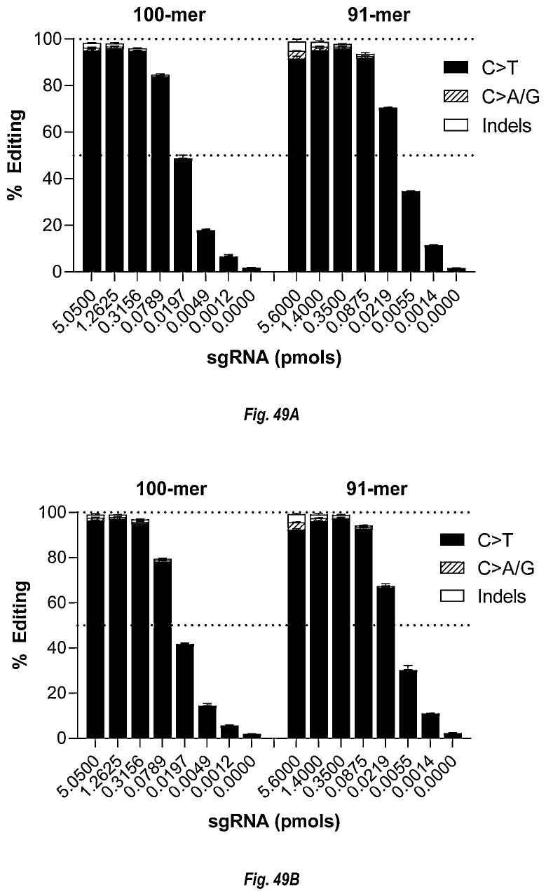

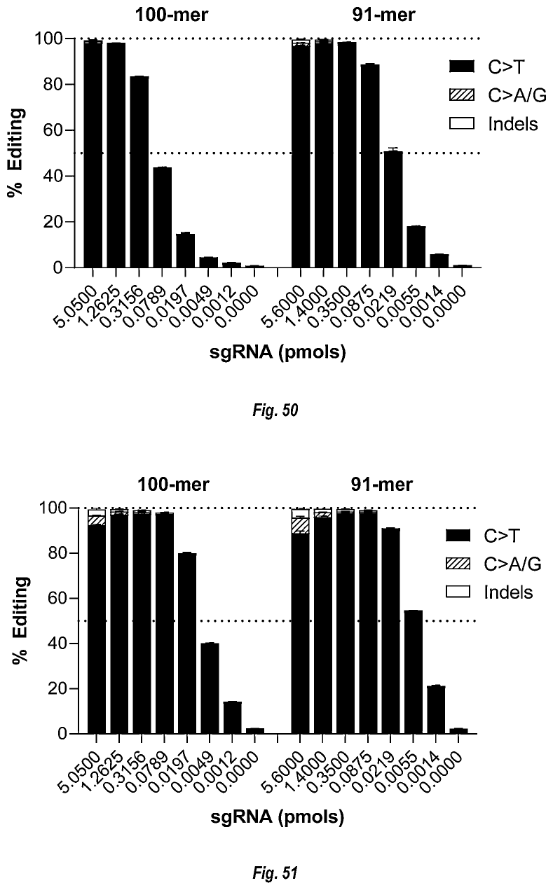

Four days post-LNP treatment, T cells were subjected to lysis, PCR amplification of each targeted locus and subsequent NGS analysis, as described in Example 1. Tables 75-80 and FIGS. 48, 50A, 50B, 51 show editing levels and the C to T editing purity in T cells treated with a decreasing mass of 100-mer or 91-mer sgRNAs targeting TRAC, TRBC1, TRBC2, CIITA, B2M or CD38.

When compared to their 100-mer versions, 91-mer sgRNAs resulted in higher editing frequencies when delivered at the same concentration. This result was observed for all 5 different sets of sgRNAs assayed by NGS, which target 6 different genomic loci. No differences in C to T editing purity were observed between 100-mer and 91-mer sgRNAs. The set of sgRNAs targeting the HLA-A gene were evaluated by flow cytometry instead of NGS due to the hyperpolymorphic nature of the HLA-A locus.

TABLE 75
Mean percent editing at the TRAC locus in T cells treated with sgRNAs in the 100-mer
(G016017) or 91-mer format (G023520).

| | TRAC | | | | | | | | | | | |
|---|---|---|---|---|---|---|---|---|---|---|---|---|
| | 100-mer | | | | | | 91-mer | | | | | |
| sgRNA | C to T | | C to A/G | | Indels | | C to T | | C to A/G | | Indels | |
| (ng) | Mean | SD | Mean | SD | Mean | SD | Mean | SD | Mean | SD | Mean | SD |
| 166.00 | 96.7 | 0.4 | 1.0 | 0.2 | 1.5 | 0.3 | 94.9 | 0.0 | 2.2 | 0.2 | 2.5 | 0.3 |
| 41.50 | 98.0 | 0.5 | 0.4 | 0.1 | 0.5 | 0.4 | 97.5 | 0.1 | 0.8 | 0.0 | 1.2 | 0.1 |
| 10.38 | 98.4 | 0.4 | 0.2 | 0.1 | 0.5 | 0.1 | 98.4 | 0.1 | 0.4 | 0.1 | 0.7 | 0.1 |
| 2.59 | 97.2 | 0.1 | 0.3 | 0.1 | 0.4 | 0.1 | 98.2 | 0.2 | 0.3 | 0.1 | 0.5 | 0.0 |
| 0.65 | 78.3 | 0.9 | 0.3 | 0.1 | 0.5 | 0.0 | 92.0 | 0.5 | 0.3 | 0.1 | 0.4 | 0.1 |
| 0.16 | 37.4 | 0.9 | 0.1 | 0.0 | 0.2 | 0.1 | 55.3 | 0.7 | 0.2 | 0.0 | 0.3 | 0.1 |
| 0.04 | 11.6 | 0.9 | 0.2 | 0.1 | 0.1 | 0.1 | 19.4 | 0.9 | 0.2 | 0.1 | 0.2 | 0.1 |
| 0.00 | 0.9 | 0.2 | 0.1 | 0.1 | 0.1 | 0.0 | 0.8 | 0.1 | 0.2 | 0.0 | 0.1 | 0.0 |

TABLE 76
Mean percent editing at the TRBC1 locus in T cells treated with sgRNAs in the 100-mer
(G016200) or 91-mer format (G023524). .

| | TRBC1 | | | | | | | | | | | |
|---|---|---|---|---|---|---|---|---|---|---|---|---|
| | 100-mer | | | | | | 91-mer | | | | | |
| sgRNA | C to T | | C to A/G | | Indels | | C to T | | C to A/G | | Indels | |
| (ng) | Mean | SD | Mean | SD | Mean | SD | Mean | SD | Mean | SD | Mean | SD |
| 166.00 | 95.1 | 0.5 | 1.3 | 0.2 | 1.9 | 0.2 | 91.6 | 1.0 | 3.4 | 0.1 | 4.0 | 0.8 |
| 41.50 | 96.2 | 0.8 | 0.6 | 0.1 | 1.3 | 0.3 | 95.3 | 0.6 | 1.5 | 0.2 | 2.1 | 0.3 |
| 10.38 | 94.6 | 0.6 | 0.4 | 0.1 | 1.0 | 0.2 | 96.0 | 0.4 | 0.8 | 0.2 | 1.1 | 0.1 |
| 2.59 | 83.5 | 0.7 | 0.5 | 0.1 | 0.7 | 0.3 | 92.1 | 0.6 | 0.6 | 0.1 | 0.9 | 0.5 |
| 0.65 | 48.1 | 2.0 | 0.4 | 0.1 | 0.3 | 0.1 | 69.8 | 0.1 | 0.4 | 0.2 | 0.4 | 0.2 |
| 0.16 | 17.3 | 0.7 | 0.3 | 0.1 | 0.3 | 0.4 | 34.0 | 0.5 | 0.4 | 0.0 | 0.3 | 0.1 |
| 0.04 | 6.2 | 1.2 | 0.3 | 0.1 | 0.2 | 0.1 | 10.9 | 0.7 | 0.5 | 0.1 | 0.1 | 0.1 |
| 0.00 | 1.3 | 0.1 | 0.4 | 0.1 | 0.2 | 0.1 | 1.2 | 0.4 | 0.3 | 0.2 | 0.1 | 0.1 |

TABLE 77
Mean percent editing at the TRBC2 locus in T cells treated with sgRNAs in the 100-mer
(G016200) or 91-mer format (G023524).

| | TRBC2 | | | | | | | | | | | |
|---|---|---|---|---|---|---|---|---|---|---|---|---|
| | 100-mer | | | | | | 91-mer | | | | | |
| sgRNA | C to T | | C to A/G | | Indels | | C to T | | C to A/G | | Indels | |
| (ng) | Mean | SD | Mean | SD | Mean | SD | Mean | SD | Mean | SD | Mean | SD |
| 166.00 | 96.5 | 0.1 | 1.2 | 0.3 | 1.3 | 0.3 | 92.4 | 0.4 | 3.2 | 0.1 | 3.7 | 0.3 |
| 41.50 | 97.3 | 0.5 | 0.7 | 0.0 | 0.9 | 0.0 | 96.1 | 0.3 | 1.3 | 0.1 | 1.7 | 0.5 |
| 10.38 | 95.3 | 0.9 | 0.6 | 0.2 | 1.0 | 0.1 | 97.1 | 0.1 | 0.6 | 0.1 | 1.2 | 0.2 |
| 2.59 | 78.3 | 1.4 | 0.6 | 0.0 | 0.6 | 0.1 | 92.8 | 0.8 | 0.7 | 0.1 | 0.7 | 0.2 |
| 0.65 | 41.1 | 1.1 | 0.3 | 0.0 | 0.4 | 0.1 | 66.5 | 2.0 | 0.4 | 0.3 | 0.5 | 0.2 |
| 0.16 | 13.9 | 1.5 | 0.3 | 0.1 | 0.2 | 0.0 | 29.7 | 2.5 | 0.3 | 0.2 | 0.3 | 0.2 |
| 0.04 | 5.1 | 0.7 | 0.3 | 0.0 | 0.3 | 0.1 | 10.3 | 0.5 | 0.4 | 0.1 | 0.3 | 0.1 |
| 0.00 | 1.5 | 0.4 | 0.4 | 0.1 | 0.2 | 0.1 | 1.8 | 0.3 | 0.4 | 0.1 | 0.2 | 0.1 |

TABLE 78
Mean percent editing at the CIITA locus in T cells treated with sgRNAs in the 100-mer
(G016086) or 91-mer format (G023521).

| | CIITA | | | | | | | | | | | |
|---|---|---|---|---|---|---|---|---|---|---|---|---|
| | 100-mer | | | | | | 91-mer | | | | | |
| sgRNA | C to T | | C to A/G | | Indels | | C to T | | C to A/G | | Indels | |
| (ng) | Mean | SD | Mean | SD | Mean | SD | Mean | SD | Mean | SD | Mean | SD |
| 166.00 | 98.1 | 0.2 | 0.7 | 0.1 | 0.6 | 0.1 | 96.9 | 0.3 | 1.3 | 0.0 | 1.4 | 0.2 |
| 41.50 | 97.3 | 0.2 | 0.4 | 0.1 | 0.3 | 0.1 | 98.1 | 0.2 | 0.6 | 0.1 | 0.8 | 0.1 |
| 10.38 | 82.9 | 0.3 | 0.3 | 0.0 | 0.2 | 0.1 | 97.5 | 0.4 | 0.5 | 0.2 | 0.4 | 0.1 |
| 2.59 | 43.3 | 0.5 | 0.3 | 0.1 | 0.2 | 0.1 | 88.0 | 1.1 | 0.4 | 0.1 | 0.3 | 0.1 |
| 0.65 | 14.5 | 0.9 | 0.3 | 0.1 | 0.1 | 0.1 | 50.4 | 1.9 | 0.3 | 0.1 | 0.1 | 0.1 |
| 0.16 | 4.3 | 0.2 | 0.2 | 0.1 | 0.0 | 0.0 | 17.7 | 0.5 | 0.3 | 0.0 | 0.1 | 0.1 |
| 0.04 | 1.8 | 0.2 | 0.3 | 0.1 | 0.1 | 0.0 | 5.7 | 0.1 | 0.2 | 0.1 | 0.1 | 0.0 |
| 0.00 | 0.6 | 0.1 | 0.2 | 0.1 | 0.1 | 0.0 | 0.8 | 0.0 | 0.2 | 0.1 | 0.1 | 0.0 |

TABLE 79
Mean percent editing at the B2M locus in T cells treated with sgRNAs in the 100-mer
(G015991) or 91-mer format (G023519).

| | B2M | | | | | | | | | | | |
|---|---|---|---|---|---|---|---|---|---|---|---|---|
| | 100-mer | | | | | | 91-mer | | | | | |
| sgRNA | C to T | | C to A/G | | Indels | | C to T | | C to A/G | | Indels | |
| (ng) | Mean | SD | Mean | SD | Mean | SD | Mean | SD | Mean | SD | Mean | SD |
| 166.00 | 92.5 | 0.3 | 4.2 | 0.2 | 2.8 | 0.4 | 89.0 | 0.9 | 6.9 | 0.5 | 3.8 | 0.3 |
| 41.50 | 97.2 | 0.2 | 1.3 | 0.2 | 1.1 | 0.1 | 96.1 | 0.4 | 2.1 | 0.1 | 1.5 | 0.4 |
| 10.38 | 97.6 | 0.4 | 0.6 | 0.1 | 0.9 | 0.1 | 97.7 | 0.1 | 0.8 | 0.0 | 1.0 | 0.1 |
| 2.59 | 96.8 | 0.1 | 0.5 | 0.0 | 0.7 | 0.1 | 97.9 | 0.1 | 0.6 | 0.0 | 0.6 | 0.1 |
| 0.65 | 79.5 | 0.9 | 0.3 | 0.0 | 0.3 | 0.0 | 90.3 | 0.3 | 0.4 | 0.0 | 0.5 | 0.1 |
| 0.16 | 39.8 | 0.3 | 0.3 | 0.0 | 0.2 | 0.1 | 54.1 | 0.4 | 0.3 | 0.1 | 0.2 | 0.0 |
| 0.04 | 13.9 | 0.3 | 0.3 | 0.1 | 0.1 | 0.0 | 20.8 | 0.8 | 0.3 | 0.0 | 0.1 | 0.1 |
| 0.00 | 2.1 | 0.2 | 0.3 | 0.0 | 0.1 | 0.0 | 2.0 | 0.4 | 0.3 | 0.0 | 0.1 | 0.0 |

TABLE 80
Mean percent editing at the CD38 locus in T cells treated with sgRNAs in the 100-mer
(G019771) or 91-mer format (G023522).

| | CD38 | | | | | | | | | | | |
|---|---|---|---|---|---|---|---|---|---|---|---|---|
| | 100-mer | | | | | | 91-mer | | | | | |
| sgRNA | C to T | | C to A/G | | Indels | | C to T | | C to A/G | | Indels | |
| (ng) | Mean | SD | Mean | SD | Mean | SD | Mean | SD | Mean | SD | Mean | SD |
| 166.00 | 92.7 | 0.6 | 1.6 | 0.2 | 5.4 | 0.4 | 91.0 | 0.6 | 2.5 | 0.2 | 6.1 | 0.5 |
| 41.50 | 93.9 | 0.4 | 0.7 | 0.1 | 5.1 | 0.3 | 93.4 | 0.5 | 1.0 | 0.0 | 5.2 | 0.5 |
| 10.38 | 93.7 | 0.1 | 0.7 | 0.1 | 4.8 | 0.2 | 94.0 | 0.4 | 0.6 | 0.1 | 4.6 | 0.2 |
| 2.59 | 88.4 | 0.9 | 0.5 | 0.1 | 3.3 | 0.3 | 92.4 | 0.7 | 0.6 | 0.1 | 3.8 | 0.3 |
| 0.65 | 54.8 | 1.6 | 0.4 | 0.0 | 1.9 | 0.3 | 67.3 | 0.3 | 0.4 | 0.1 | 2.3 | 0.0 |
| 0.16 | 19.7 | 0.4 | 0.5 | 0.1 | 0.7 | 0.1 | 27.8 | 0.4 | 0.5 | 0.1 | 1.0 | 0.2 |
| 0.04 | 5.9 | 0.5 | 0.5 | 0.0 | 0.3 | 0.1 | 9.1 | 0.6 | 0.4 | 0.1 | 0.4 | 0.1 |
| 0.00 | 0.4 | 0.0 | 0.4 | 0.1 | 0.1 | 0.0 | 0.5 | 0.1 | 0.4 | 0.0 | 0.0 | 0.0 |

### Example 30.4. Evaluation of Receptor Knockout by Flow Cytometry

Seven days post LNP treatment, T cells were assayed by flow cytometry to evaluate receptor knockout. T cells were incubated with a fixable viability dye (Beckman Coulter, Cat. C36628) and an antibody cocktail targeting the following molecules: CD3 (Biolegend, Cat. 317336), CD4 (Biolegend, Cat. 317434) and CD8 (Biolegend, Cat. 301046), B2M (Biolegend, Cat. 316306), CD38 (Biolegend, Cat. 303516), HLA-A2 (Biolegend, Cat. 343304) and HLA-DR, DP, DQ (Biolegend, Cat. 361714). Cells were subsequently washed, analyzed on a Cytoflex LX instrument (Beckman Coulter) using the FlowJo software package. T cells were gated on size, viability and CD8 positivity before expression of any markers was determined. The resulting data was plotted on GraphPad Prism v. 9.0.2 and analyzed using a variable slope (four parameter) non-linear regression.

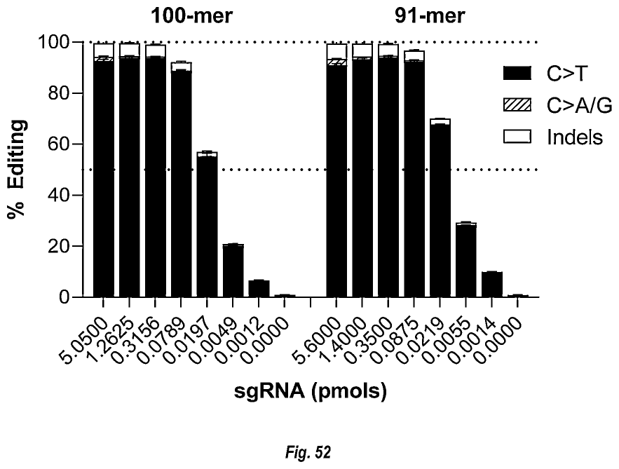

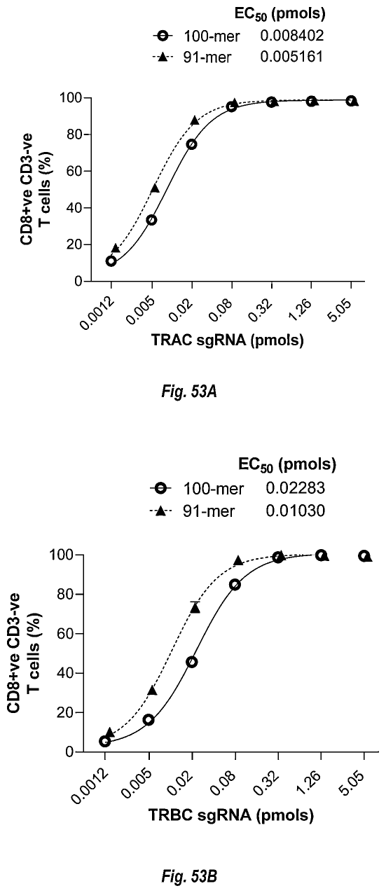

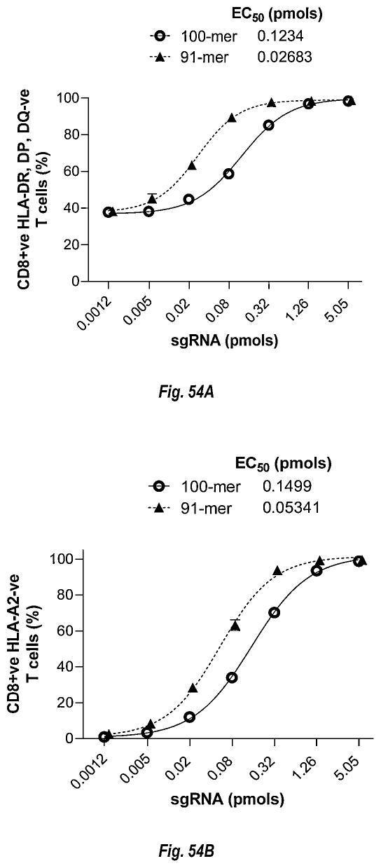

As shown in Tables 81-83 and FIGS. 52, 53A-55B, all 91-mer sgRNAs tested outperformed their 100-mer versions. As show in Table 84, this increase in potency was variable, with a 4.6-fold increase observed for CIITA and a 1.14-fold increase observed for B2M. Among the 6 targets tested, those with a lower potency (i.e., higher EC50) in the 100-mer format (CIITA and HLA-A) seem to benefit the most from usage of 91-mer sgRNAs.

TABLE 81
Mean percentage of CD8+ T cells that are negative for CD3 surface receptors
following sgRNA targeting TRAC or TRBC1/2 in the 100-mer or 91-mer formats.

| | TRAC (CD3-) | | | | | TRBC1/2 (CD3-) | | | |
|---|---|---|---|---|---|---|---|---|---|
| sgRNA | 100-mer | | 91-mer | | sgRNA | 100-mer | | 91-mer | |
| (ng) | Mean | SD | Mean | SD | (ng) | Mean | SD | Mean | SD |
| 166.00 | 98.4 | 0.3 | 98.2 | 0.3 | 166.00 | 99.6 | 0.3 | 99.1 | 0.3 |
| 41.50 | 98.1 | 0.1 | 98.5 | 0.5 | 41.50 | 99.8 | 0.1 | 99.6 | 0.1 |
| 10.38 | 97.7 | 0.2 | 98.0 | 0.4 | 10.38 | 98.8 | 0.4 | 99.9 | 0.1 |
| 2.59 | 95.2 | 0.4 | 97.4 | 0.6 | 2.59 | 84.9 | 1.1 | 97.4 | 0.6 |
| 0.65 | 74.7 | 1.0 | 88.0 | 0.8 | 0.65 | 45.7 | 1.6 | 73.5 | 2.8 |
| 0.16 | 33.5 | 2.4 | 51.0 | 1.0 | 0.16 | 16.4 | 0.9 | 31.6 | 0.8 |
| 0.04 | 11.0 | 0.7 | 18.3 | 0.8 | 0.04 | 5.5 | 0.4 | 10.2 | 0.5 |
| 0.00 | 1.1 | 0.2 | 1.3 | 0.2 | 0.00 | 1.2 | 0.2 | 1.2 | 0.1 |

TABLE 82
Mean percentage of CD8+ T cells that are negative for HLA-DR, DP, DQ or
HLA-A2 surface receptors following treatment sgRNA targeting CIITA or HLA-A,
respectively, in the 100-mer or 91-mer formats.

| | CIITA (HLA-DR, DP, DQ-) | | | | | HLA-A (HLA-A2-) | | | |
|---|---|---|---|---|---|---|---|---|---|
| sgRA | 100-mer | | 91-mer | | sgRNA | 100-mer | | 91-mer | |
| (ng) | Mean | SD | Mean | SD | (ng) | Mean | SD | Mean | SD |
| 166.00 | 98.3 | 0.2 | 98.7 | 0.2 | 166.00 | 98.8 | 0.1 | 99.6 | 0.2 |
| 41.50 | 96.9 | 0.7 | 98.4 | 0.3 | 41.50 | 93.6 | 0.8 | 99.2 | 0.4 |
| 10.38 | 85.2 | 0.7 | 97.7 | 0.3 | 10.38 | 70.2 | 1.0 | 93.8 | 1.4 |
| 2.59 | 58.7 | 0.2 | 89.4 | 1.3 | 2.59 | 34.0 | 2.1 | 63.2 | 3.0 |
| 0.65 | 44.8 | 1.1 | 63.4 | 0.5 | 0.65 | 12.1 | 1.3 | 28.5 | 1.2 |
| 0.16 | 38.2 | 1.6 | 45.2 | 2.6 | 0.16 | 3.3 | 0.2 | 8.3 | 0.6 |
| 0.04 | 37.8 | 1.0 | 38.2 | 0.5 | 0.04 | 0.9 | 0.3 | 2.6 | 0.5 |
| 0.00 | 35.1 | 2.5 | 37.6 | 1.7 | 0.00 | 0.1 | 0.0 | 0.3 | 0.2 |

TABLE 83
Mean percentage of CD8+ T cells that are negative for B2M or CD38 surface
receptors following treatment with sgRNAs targeting B2M or CD38, respectively in the
100-mer or 91-mer formats.

| | B2M (B2M-) | | | | | CD38 (CD38-) | | | |
|---|---|---|---|---|---|---|---|---|---|
| sgRNA | 100-mer | | 91-mer | | sgRNA | 100-mer | | 91-mer | |
| (ng) | Mean | SD | Mean | SD | (ng) | Mean | SD | Mean | SD |
| 166.00 | 98.0 | 0.4 | 98.3 | 0.2 | 166.00 | 99.8 | 0.2 | 99.8 | 0.1 |
| 41.50 | 98.2 | 0.0 | 98.4 | 0.4 | 41.50 | 99.6 | 0.1 | 99.9 | 0.1 |
| 10.38 | 94.5 | 0.7 | 97.2 | 0.3 | 10.38 | 98.4 | 0.3 | 99.5 | 0.1 |
| 2.59 | 68.3 | 3.2 | 81.4 | 3.4 | 2.59 | 87.4 | 1.9 | 94.3 | 0.9 |
| 0.65 | 8.4 | 6.8 | 11.4 | 2.6 | 0.65 | 47.0 | 4.3 | 60.0 | 1.9 |
| 0.16 | 0.4 | 0.3 | 0.5 | 0.1 | 0.16 | 20.5 | 4.9 | 23.8 | 1.6 |
| 0.04 | 0.4 | 0.6 | 0.2 | 0.1 | 0.04 | 16.5 | 10.1 | 12.5 | 0.6 |
| 0.00 | 0.9 | 0.7 | 1.6 | 0.9 | 0.00 | 17.2 | 4.1 | 13.7 | 0.7 |

TABLE 84
Amount (pmol) of sgRNAs that lead to a 50% loss of receptor expression in
the surface of CD8 + T cells (EC50s). The far right column shows the fold-increase in
potency achieved by 91-mer sgRNAs when compared to their 100-mer with the same
guide sequence.

| | | | | | EC50 shift |
|---|---|---|---|---|---|
| | 100-mer | | 91-mer | | (100-mer/91- |
| Gene target | sgRNA ID | EC50 (pmols) | sgRNA ID | EC50 (pmols) | mer) |
| TRAC | G016017 | 0.008 | G023520 | 0.005 | 1.63 |
| TRBC1/2 | G016200 | 0.023 | G023524 | 0.010 | 2.22 |
| CIITA | G016086 | 0.123 | G023521 | 0.027 | 4.60 |
| B2M | G015991 | 0.055 | G023519 | 0.048 | 1.14 |
| CD38 | G019771 | 0.027 | G023522 | 0.020 | 1.41 |
| HLA-A | G021209 | 0.150 | G023523 | 0.053 | 2.81 |

### Example 31 Base Editing Impact of UGI mRNA Dose on In-Vivo Editing

In vivo liver editing profiles were evaluated using fixed doses of BC22n (SEQ ID No: 1) and guide RNA targeting ANAPC5 (G019427) along with a serial dilution of UGI mRNA (SEQ ID No: 34).

### Example 31.1 In Vivo Editing as Assayed by NGS

Fifteen commercially available CD-1 female mice ranging from 6-10 weeks of age (n=3 per group) were used in this study. Animals were weighed pre-dose for dosing calculations. Each RNA species was formulated separately in an LNP. LNPs were formulated generally as described in Example 1. LNPs contained ionizable Lipid A, cholesterol, DSPC, and PEG2k-DMG in a 50:38:9:3 molar ratio, respectively. The lipid nucleic acid assemblies were formulated with a lipid amine to RNA phosphate (N:P) molar ratio of about 6. LNPs encapsulated a single RNA species, either G019427, BC22n mRNA (SEQ ID No: 1), or UGI mRNA (SEQ ID No. 34).

LNPs encapsulating base editor mRNA and LNPs encapsulating sgRNA were mixed and administered simultaneously at fixed doses of 0.2 mpk and 0.1 mpk RNA by weight, respectively (editor mRNA and sgRNA) along with UGI mRNA doses of either 0.0, 0.03, 0.1, and 0.3 mpk RNA by weight. The negative control group was dosed with TSS buffer only. Formulations were administered intravenously via tail vein injection according to the doses listed in Table 85.

TABLE 85
Animal groups and the respective LNPs

| | | Editor | | UGI |
|---|---|---|---|---|
| | | mRNA | sgRNA | mRNA |
| Sample | LNP cargos | (mg/kg) | (mg/kg) | (mg/kg) |
| TSS | None | 0 | 0 | 0 |
| 0.0 MPK UGI | BC22n mRNA | 0.2 mg/kg | 0.1 mg/kg | n/a |
| 0.03 MPK UGI | UGI mRNA | | | 0.03 mg/kg |
| 0.1 MPK UGI | G019427 | | | 0.1 mg/kg |
| 0.3 MPK UGI | | | | 0.3 mg/kg |

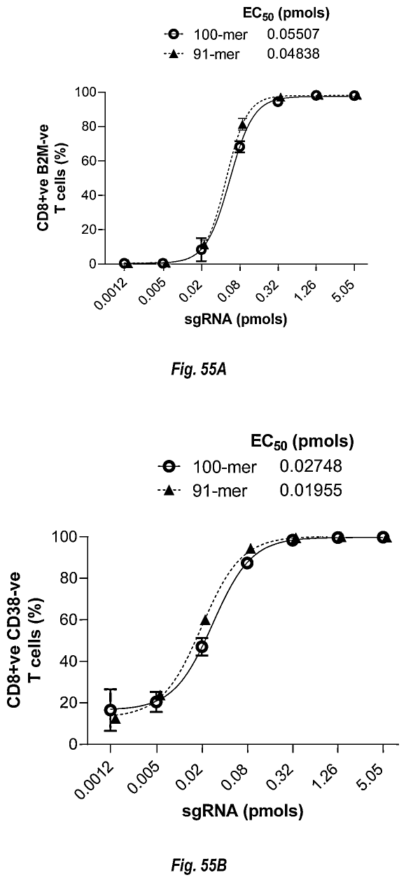

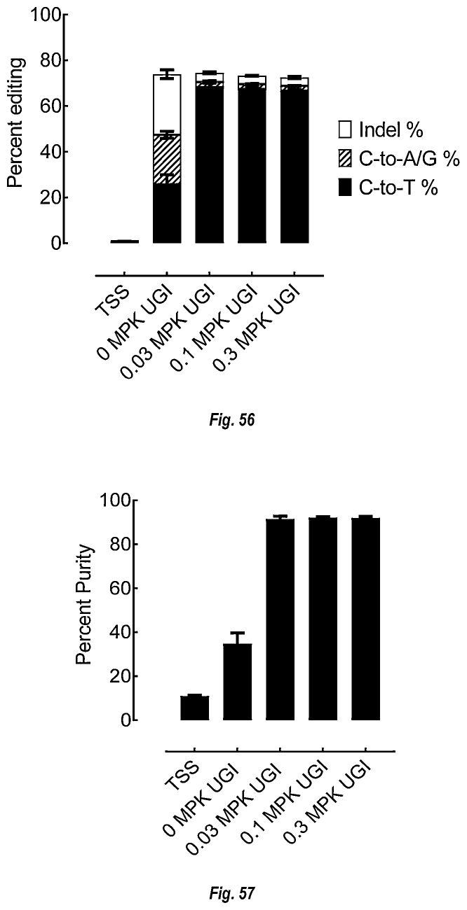

Animals were periodically observed for adverse effects for at least 24 hours post-dose. Six days after treatment, animals were euthanized by cardiac puncture under isoflurane anesthesia; liver tissue were collected for downstream analysis. Liver punches weighing between 5 and 15 mg were collected for isolation of genomic DNA. Genomic DNA samples were analyzed with NGS sequencing as described in Example 1. Editing data and percent C to T purity are shown in Table 86 and FIGS. 56-57.

TABLE 86
Mean percent editing at the ANAPC5 locus in mouse liver with
increasing amounts of UGI mRNA.

| UGI | | | | | | | % C to T | |
|---|---|---|---|---|---|---|---|---|
| mRNA | % C to T | | % C to A/G | | Indel | | Purity | |
| (mpK) | Mean | SD | Mean | SD | Mean | SD | Mean | SD |
| TSS | 0.10 | 0.00 | 0.71 | 0.03 | 0.11 | 0.10 | 11.01 | 0.38 |
| 0 | 25.77 | 4.18 | 21.64 | 1.52 | 24.29 | 26.51 | 34.80 | 4.90 |
| 0.03 | 68.20 | 2.38 | 2.30 | 0.54 | 4.11 | 4.02 | 91.50 | 1.27 |
| 0.1 | 67.47 | 0.55 | 2.04 | 0.25 | 3.92 | 3.71 | 92.15 | 0.39 |
| 0.3 | 66.67 | 0.67 | 2.17 | 0.14 | 3.88 | 3.63 | 91.99 | 0.66 |

### Example 32. Determination of BC22n to UGI Ratio In Vitro

Protein expression levels are determined using HiBiT tagged versions of a protein. To determine the relative amounts of base editor protein and UGI protein expressed for efficient base editing with high C to T purity, parallel editing experiments are performed one of which uses an mRNA encoding a HiBiT-tagged base editor and the other of which uses an mRNA encoding a HiBiT-tagged UGI.

Cell preparation, engineering and editing assays are performed as in Example 9. One arm of the experiment (editor tagged) transfects with an mRNA encoding a HiBiT-tagged BC22n (SEQ ID No. 4) and an mRNA UGI (SEQ ID No: 34). Another arm of the experiment (UGI tagged) transfects with an mRNA encoding base editor (SEQ ID No: 1) and an mRNA encoding a Hibit-tagged UGI.

Twenty-four hours after transfection, the number of live T cells per sample is determined using a cell titer-glo assay (Promega Cat No. G7571) and the number of HiBiT tagged proteins is measured using a Nano-Glo® HiBiT lytic detection assay (Promega Cat No. N3040). NGS sequencing is performed 96 hours post-transfection to assess total editing and C to T purity levels. BC22n and UGI protein levels is plotted against total editing levels to determine the minimum number of each proteins cell that is required to achieve saturating editing levels and C to T purity.

The optimal ratio of BC22n to UGI proteins is calculated by dividing the minimum number of BC22 proteins per cell that is required to achieve saturating editing levels by the minimum number of UGI proteins per cell that is required to achieve maximum C to T purity.

Similar methods could be used to calculate the ratio of base editor to UGI protein that use different cell types, different guides, or different transfection methods.

### Example 33. In Vivo UGI Titration with Lower Doses

Base editing is performed in mice to assess overall editing efficiency and C to T purity with lower levels of UGI mRNA. Experimental details are as described in Example 30 with the following exceptions. LNPs encapsulating BC22n mRNA and LNPs encapsulating sgRNA are mixed at fixed doses of 0.2 mg/kg and 0.1 mg/kg of RNA cargos respectively, and combined with UGI mRNA doses of either 0.0, 0.0001, 0.001, 0.003, 0.01 0.03, 0.1, and 0.3 mg/kg. The negative control group is a TSS treated animal. Formulations are administered intravenously via tail vein injection. Six days after treatment, animals are euthanized by cardiac puncture under isoflurane anesthesia; liver tissue are collected for downstream analysis. Liver punches weighing between 5 and 15 mg are collected for isolation of genomic DNA and total RNA. Genomic DNA samples are analyzed with NGS sequencing as described in Example 1 to determine the minimum UGI mRNA dose required for maximum C-to-T editing purity in mouse liver.

### Example 34. Cytotoxic Susceptibility of Engineered T Cells

Engineered T cells are assayed for cytotoxic susceptibility when targeted by natural killer (NK) cells.

NK cells (Stemcell Technologies) are thawed and resuspended at a cell concentration of 1×10\hat{ }6 cells/ml into T cell growth media (TCGM) composed of OpTmizer TCGM and further supplemented with 100 U/mL of recombinant human interleukin-2 (Peprotech, Cat. 200-02), 5 ng/ml IL-7 (Peprotech, Cat. 200-07), 5 ng/ml IL-15 (Peprotech, Cat. 200-15). Cells are incubated at 37° C. for 24 hours.

Twenty-four hours post thaw, the NK cells are labelled with 0.5 μM Cell Trace Violet as follows: a vial of Cell Trace Violet (CellTrace™ Violet Cell Proliferation Kit, for flow cytometry, Cat. C34571) is reconstituted in DMSO from the kit to give a 5 mM stock concentration. Two μL of CTV stock is diluted with 18 μL Phosphate-Buffered Saline (Corning, Cat. 21-040-CV) to obtain a concentration of 0.5 mM. NK cells are centrifuged at 500×g for 5 minutes, the media is aspirated, and cells are resuspended in Phosphate-Buffered Saline (PBS) at a concentration of 1×10\hat{ }6 cells/mL such that the final concentration of CTV dye is 0.5 μM. The cells are mixed with CTV (Cell Trace Violet) dye solution incubated at 37° C. for 20 minutes. Unbound dye is quenched by the addition of TCGM and incubated for 5 minutes. The cells are centrifuged at 500×g for 5 minutes. Cells are resuspended in TCGM supplemented with 100 U/mL of recombinant human interleukin-2 (Peprotech, Cat. 200-02), 5 ng/ml IL-7 (Peprotech, Cat. 200-07), 5 ng/ml IL-15 (Peprotech, Cat. 200-15) at a concentration of 2×10\hat{ }6 cells/mL. To test a range of effector:target (E:T) ratios, CTV-labelled NK cells aliquoted in 100 ul of media in a 6 point, 2 fold serial dilution with the highest number of cells being 2×10\hat{ }5 cells Media only samples are included as negative controls.

T cells are engineered using BC22n and UGI mRNA as described in Example 14 using the G023523 targeting HLA-A or G015991 targeting B2M. Unedited (WT) T cells, Unstained, LD heat killed and 7-AAD FMO (2×10\hat{ }⁴ unlabelled NK cells and 2×10\hat{ }⁴ WT T cells) and CTV+ (2×10\hat{ }5 CTV labelled NK cells) are also included as controls. T cells are resuspended at a density of 2×10\hat{ }5 cells of TCGM composed of OpTmizer TCGM and further supplemented with 100 U/mL of recombinant human interleukin-2 (Peprotech, Cat. 200-02), 5 ng/ml IL-7 (Peprotech, Cat. 200-07), 5 ng/ml IL-15 (Peprotech, Cat. 200-15). Twenty thousand T cells are added each well of NK cells and media controls. Cells are incubated at 37° C. for 24 hours.

At twenty-four hours, half of the volume of the cells from the LD Heat Killed well are heat killed and transferred back to the same well in the assay plate. Cells are centrifuged and resuspended in in 80 μL of a 1:200 v/v solution of 7-AAD (BD Biosciences, Cat. 559925) in FACS buffer (PBS+2% FBS (Gibco, Cat. A31605-02)+2 mM EDTA (Invitrogen, Cat. 15-575-020). Data for specific lysis of T cells is acquired by flow cytometry using a Cytoflex LX instrument (Beckman Coulter) and analyzed using the FlowJo software package. Gates are first drawn on the CTV negative population to gate out the NK cells, followed by gating on singlets after which a gate is drawn on the 7-AAD negative population to gate for the live T cells. The percent lysis of T cells is calculated by subtracting the live cell percentage from 100.

### Example 35. This Paragraph is Intentionally Left Blank

### Example 36. Protein Expression of Base Editor and UGI in Primary Human Hepatocytes

Base editing was performed with mRNAs encoding proteins with fused HiBiT tags in primary human hepatocytes to determine the relative amounts of base editor and UGI protein enzymatic units appropriate for high efficiency editing with high C to T purity.

Messenger RNAs encoding BC22n with a C-terminal HiBiT tag (BC22n-HiBIT, SEQ ID NO: 4), BC22-2XUGI (2 tandem copies of UGI in cis) with a C-terminal HiBiT tag (BC22-2XUGI-HibIT, SEQ ID NO: 314), and UGI with a C-terminal HiBiT tag (UGI-HiBiT, SEQ ID NO: 316), were transfected into primary human hepatocytes (PHH) in dose response with a fixed concentration of a guide RNA targeting B2M (G015991; SEQ ID NO: 179) to determine the minimum number of intracellular base editor and UGI protein copies to perform base editing with high activity (total editing %=2×EC90) and high C-to-T purity (C to T purity %=2×EC90). BC22n-HiBiT was titrated across fixed concentrations of B2M guide and UGI (SEQ ID NO: 34), UGI-HiBiT was titrated across fixed concentrations of B2M guide and BC22n (SEQ ID NO: 1), and BC22-2XUGI-HiBiT was titrated across a fixed concentration of B2M guide without any additional UGI mRNA in trans. A HiBiT lytic assay (Promega cat #N3040) was performed twenty hours post transfection to determine intracellular protein levels at an early timepoint. Those protein levels were then related to endpoint editing at the B2M locus, derived from NGS data, ninety-six hours post transfection.

### Example 36.1 Cell Preparation and Transfection

PHH cells (ThermoFisher, Lot HU8284) were thawed and recovered in CHRMs media (Gibco, cat #CM7000). Cells were resuspended in Primary Hepatocyte Plating Media (Consisting of William's E media (Gibco, Cat #A1217601) and primary hepatocyte plating supplements (Gibco, Cat #CM3000)) before being plated in collagen-coated 96-well plates at a density of 30,000 cells/well for twenty-four hours. Cells were washed, and fresh primary hepatocyte maintenance media was added.

Lipofection reagent was prepared as described in Example 1, with a molar ratio of 50/9/38/3 of Lipid A, DSPC, cholesterol, and PEG2k-DMG respectively. Each RNA species (base editor mRNA, UGI mRNA, or gRNA G015991 (SEQ ID NO: 179)) was individually bulk-mixed with lipofection reagent at a lipid amine to RNA phosphate (N:P) molar ratio of about 6. The resulting bulk-mixed lipoplex material was pre-incubated with 10% FBS (Gibco, Cat #A3160501) in Primary Hepatocyte Maintenance Media (Consisting of William's E media (Gibco, Cat #A1217601) and primary hepatocyte maintenance supplements (Gibco, Cat #CM4000)), for 15 minutes before addition to hepatocytes.

For the BC22n-HiBiT titration, each well received three components in a final volume of 100 uL: BC22n-HiBiT mRNA ranging from 100 ng to 0 ng, 11 ng of UGI mRNA and 2 pmols of G015991 (B2M) as described in Table 87. For the UGI-HiBiT titration each well received three components in a final volume of 100 uL: UGI-HiBiT mRNA ranging from 100 ng to 0 ng, 33 ng of BC22n mRNA and 2 pmols of G015991 (B2M) as described in Table 88. For the BC22-2XUGI-HiBiT titration each well received three components in a final volume of 100 uL: BC22-2XUGI-HiBiT mRNA ranging from 100 ng to 0 ng, 11 ng of UGI mRNA and 2 pmols of G015991 (B2M) as described in Table 89.

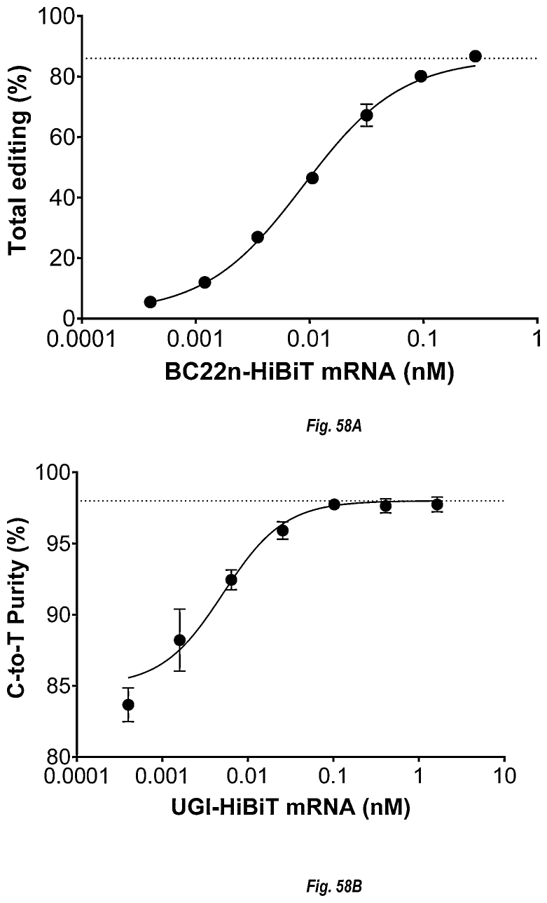

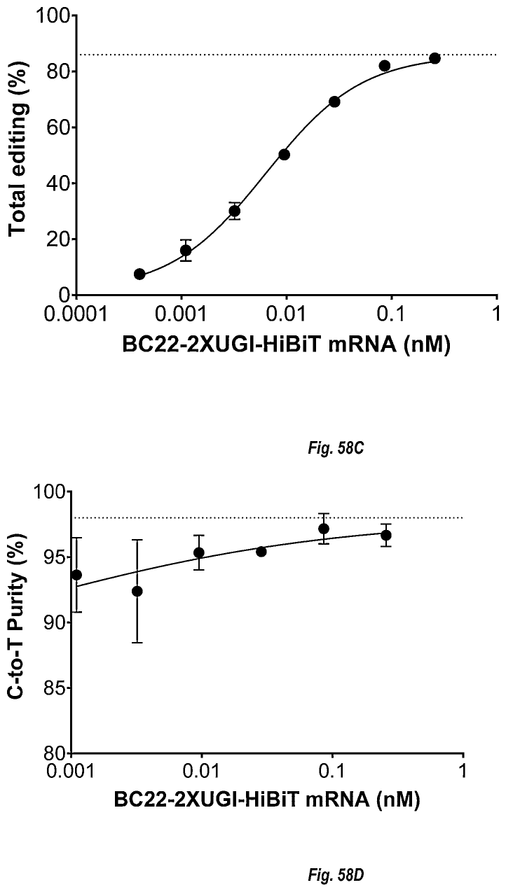

Ninety-six hours post transfection, separate PHH replicate plates that had been transfected at the same time as the HiBiT plates were subjected to lysis, PCR amplification of the B2M locus, and subsequent NGS analysis, as described in Example 1. All experiments were performed in biological triplicate. Background C-to-T, C-to-A/G and indel edits (all <2%) were subtracted from all wells by calculating the average background rates from a set of untreated wells. Editing results for different BC22n-HiBiT mRNA concentrations are shown in Table 87, and total editing at different BC22n-HiBiT mRNA concentrations are shown in FIG. 58A. Editing results for different UGI-HiBiT mRNA concentrations are shown in Table 88 and C-to-T purity at different UGI-HiBiT mRNA concentrations are shown in FIG. 58B. Editing results for different BC22-2XUGI-HiBiT sRNA concentrations are shown in Table 89. Total editing and C-to-T purity with different BC22-2XUGI-HiBiT mRNA concentrations are shown in FIG. 58C and FIG. 58D, respectively.

To facilitate comparisons between mRNAs of different sizes, all figures display mRNA doses in terms of molarity. For these purposes, the molecular weight of BC22n mRNA was considered as 1,724.25 kDa, BC22-2X-UGI mRNA as 1915.12 kDa, UGI mRNA as 287.58 kDa and sgRNAs as 29.66 kDa. The addition of a HiBiT tag to any miRNA increased its molecular weight by 18.24 kDa.

TABLE 87
Mean editing in PHH with increasing doses of BC22n-HiBiT mRNA.

| | | | | | | | | | Total | | C-to-T | |
|---|---|---|---|---|---|---|---|---|---|---|---|---|
| Dose | Dose | | C-to-T | | C-to-A/G | | Indels | | editing | | purity | |
| (ng) | (nM) | n | Mean | SD | Mean | SD | Mean | SD | Mean | SD | Mean | SD |
| 100.00 | 0.2857 | 3 | 84.87 | 1.37 | 0.95 | 0.28 | 0.80 | 0.61 | 86.74 | 1.83 | 97.99 | 0.93 |
| 33.00 | 0.0952 | 3 | 78.63 | 0.70 | 0.73 | 0.19 | 0.93 | 0.29 | 80.08 | 0.94 | 97.93 | 0.50 |
| 11.00 | 0.0317 | 3 | 66.33 | 3.72 | 0.46 | 0.24 | 0.72 | 0.39 | 67.19 | 3.67 | 98.27 | 0.39 |
| 3.70 | 0.0106 | 3 | 45.33 | 1.25 | 0.19 | 0.21 | 0.40 | 0.21 | 46.43 | 1.70 | 98.72 | 0.25 |
| 1.23 | 0.0035 | 3 | 26.57 | 0.99 | 0.32 | 0.29 | 0.90 | 0.58 | 26.92 | 1.29 | 95.69 | 2.67 |
| 0.41 | 0.0012 | 3 | 11.77 | 0.61 | 0.12 | 0.21 | 0.03 | 0.05 | 11.96 | 0.72 | 98.75 | 1.55 |
| 0.14 | 0.0004 | 3 | 5.43 | 0.85 | 0.00 | 0.00 | 0.08 | 0.14 | 5.47 | 0.82 | 98.58 | 2.45 |
| 0.00 | 0.0000 | 3 | 0.09 | 0.13 | 0.07 | 0.13 | 0.03 | 0.03 | 1.25 | 0.55 | NA | NA |

TABLE 88
Mean editing in PHH with increasing doses of UGI-Hibit mRNA.

| | | | | | | | | | Total | | C-to-T | |
|---|---|---|---|---|---|---|---|---|---|---|---|---|
| Dose | Dose | | C-to-T | | C-to-A/G | | Indels | | editing | | purity | |
| (ng) | (nM) | n | Mean | SD | Mean | SD | Mean | SD | Mean | SD | Mean | SD |
| 100.00 | 1.6400 | 3 | 81.10 | 3.33 | 0.80 | 0.13 | 1.09 | 0.42 | 83.32 | 3.34 | 97.74 | 0.52 |
| 25.00 | 0.4100 | 1 | 80.73 | 2.17 | 0.52 | 0.32 | 1.43 | 0.11 | 82.40 | 1.81 | 97.64 | 0.49 |
| 6.25 | 0.1025 | 3 | 77.10 | 3.71 | 0.64 | 0.55 | 1.15 | 0.61 | 79.19 | 4.40 | 97.74 | 0.39 |
| 1.56 | 0.0256 | 3 | 78.63 | 2.72 | 1.92 | 0.22 | 1.45 | 0.53 | 83.53 | 3.42 | 95.91 | 0.61 |
| 0.39 | 0.0064 | 3 | 74.87 | 1.88 | 3.15 | 0.04 | 2.98 | 0.71 | 82.23 | 1.91 | 92.45 | 0.70 |
| 0.10 | 0.0016 | 3 | 73.60 | 2.70 | 5.62 | 1.25 | 4.21 | 0.53 | 85.06 | 2.51 | 88.22 | 2.18 |
| 0.02 | 0.0004 | 3 | 67.33 | 0.46 | 7.30 | 1.64 | 5.84 | 0.61 | 80.15 | 1.21 | 83.67 | 1.18 |
| 0.00 | 0.0000 | 3 | 71.47 | 2.72 | 6.99 | 1.28 | 5.51 | 0.63 | 79.80 | 3.99 | 85.15 | 1.16 |

TABLE 89
Mean editing in PHH with increasing doses of BC22-2XUGI-Hibit mRNA.

| | | | | | | | | | Total | | C-to-T | |
|---|---|---|---|---|---|---|---|---|---|---|---|---|
| Dose | Dose | | C-to-T | | C-to-A/G | | Indels | | editing | | purity | |
| (ng) | (nM) | n | Mean | SD | Mean | SD | Mean | SD | Mean | SD | Mean | SD |
| 100.00 | 0.2575 | 3 | 81.90 | 2.37 | 1.47 | 0.87 | 1.34 | 0.20 | 84.29 | 1.23 | 96.67 | 0.85 |
| 33.00 | 0.0858 | 3 | 79.70 | 0.49 | 1.41 | 0.81 | 0.92 | 0.37 | 82.58 | 1.13 | 97.17 | 1.15 |
| 11.00 | 0.0286 | 3 | 65.93 | 0.75 | 1.70 | 0.30 | 1.47 | 0.19 | 68.52 | 0.96 | 95.41 | 0.20 |
| 3.70 | 0.0095 | 3 | 47.90 | 1.01 | 1.45 | 0.72 | 0.89 | 0.29 | 50.21 | 0.91 | 95.34 | 1.32 |
| 1.23 | 0.0032 | 3 | 27.77 | 2.64 | 1.45 | 0.74 | 0.86 | 0.60 | 29.52 | 2.34 | 92.39 | 3.92 |
| 0.41 | 0.0011 | 3 | 14.90 | 3.07 | 0.79 | 0.35 | 0.30 | 0.35 | 15.71 | 3.41 | 93.63 | 2.84 |
| 0.14 | 0.0004 | 3 | 7.30 | 0.74 | 0.19 | 0.15 | 0.02 | 0.02 | 7.51 | 0.86 | 97.33 | 1.89 |
| 0.00 | 0.0000 | 3 | 0.09 | 0.13 | 0.06 | 0.10 | 0.02 | 0.03 | 0.15 | 0.10 | NA | NA |

### Example 36.2 Evaluation of Intracellular Protein Levels

Twenty hours post transfection, protein levels were detected using the Nano-Glo® HiBiT Lytic Detection System (Promega cat #N3040) according to the manufacture's protocol. Promega #N3010, a commercially available control protein with a HiBiT tag (of known concentration and known molecular weight) was serially diluted (1:5) in PBS and spiked into wells containing PHH that had not been transfected as a reference control. One hundred μL of reconstituted HiBiT lytic reagent was added to the standard wells and all other experiment wells. Lysates were moved to white-walled plates, and relative luminescence units (RLU) were read out by the CLARIstar plus (BMG Labtech) plate reader with gain set at 3,600. Background signal was subtracted from all wells, and the number of protein copies per well for each sample was calculated using the linear regression equation derived by the HiBiT standard. HiBiT quantitation is presented in Table 90 and normalized to the expression level of the 3.7 ng BC22-2XUGI-HiBiT mRNA sample.

TABLE 90
Mean protein expression (arbitrary units) for Hibit-tagged proteins.

| Hibit mRNA | Dose (ng) | Dose (nM) | Mean | SD |
|---|---|---|---|---|
| BC22n-HiBIT | 100.00 | 0.2857 | 22.59 | 2.05 |
| | 33.00 | 0.0952 | 10.07 | 1.04 |
| | 11.00 | 0.0317 | 4.12 | 0.59 |
| | 3.70 | 0.0106 | 1.09 | 0.02 |
| UGI-HIBIT | 100.00 | 1.6400 | 4063.15 | 532.85 |
| | 25.00 | 0.4100 | 993.10 | 76.48 |
| | 6.25 | 0.1025 | 254.04 | 26.29 |
| | 1.56 | 0.0256 | 67.16 | 7.73 |
| | 0.39 | 0.0064 | 21.66 | 1.83 |
| BC22-2XUGI-HIBIT | 100.00 | 0.2575 | 25.82 | 2.08 |
| | 33.00 | 0.0858 | 9.13 | 1.15 |
| | 11.00 | 0.0286 | 2.87 | 0.72 |
| | 3.70 | 0.0095 | 1.00 | 0.27 |

The data in Table 90 were used to generate hyperbolic curves and interpolate the protein units expressed at the minimum dose required to achieve saturating editing or C-to-T purity levels. The minimum dose as saturating levels is defined here as twice the EC90 (2×EC90), the concentration required to achieve 90% total editing or C-to-T purity, respectively, for each mRNA tested. Table 91 shows the 2×EC90 doses (in ng and nM) of the mRNAs tested in this experiment and the BC22 and UGI peptide units and their ratios at these doses. For base editors and UGI-HiBiT, protein units are equal to peptide units. UGI peptide units for BC22-2XUGI were calculated by multiplying the BC22-2XUGI protein units by a factor of 2. For the samples edited with BC22n and UGI expressed as separate, unfused proteins, the ratio of BC22n peptide units at total editing 2×EC90 to the UGI peptide units at C to T purity 2×EC90 was approximately 1:10. For the samples edited with BC22-2XUGI, the ratio of base editor peptide units at total editing 2×EC90 to the UGI peptide units at C to T purity 2×EC90 was approximately 1:5.

TABLE 91
2X EC90 for mRNA and protein at relevant editing readouts.

| | | mRNA | mRNA | Peptide |
|---|---|---|---|---|
| mRNA | Editing Readout | (ng) | (nM) | units |
| BC22n-HiBIT | total editing | 53.97 | 0.16 | 14.94 |
| UGI-HIBIT | C-to-T purity | 3.85 | 0.06 | 156.38 |
| BC22-2XUGI-HIBIT | total editing | 49.89 | 0.13 | 13.39 |
| | (base editor) | | | |
| | C-to-T purity (UGI) | 139.23 | 0.36 | 70.14 |

### Example 37. Protein Expression of Base Editor and UGI in T Cells

Base editing was performed with mRNAs encoding proteins with fused HiBiT tags in isolated human T cells to determine the relative amounts of base editor and UGI protein enzymatic units appropriate for high efficiency editing with high C to T purity.

### Example 37.1. T Cell Preparation

Healthy human donor apheresis was obtained commercially (Hemacare), and cells were washed, re-suspended in CliniMACS® PBS/EDTA buffer (Miltenyi Biotec Cat. 130-070-525) and processed in a MultiMACS™ Cell 24 Separator Plus device (Miltenyi Biotec). T cells were isolated via positive selection using a Straight from Leukopak® CD4/CD8 MicroBead kit, human (Miltenyi Biotec Cat. 130-122-352). T cells were aliquoted and cryopreserved for future use in Cryostor® CS10 (StemCell Technologies Cat. 07930).

Upon thaw, T cells were plated at a density of 1.0×10\hat{ }6 cells/mL in T cell growth media (TCGM) composed of CTS OpTmizer T Cell Expansion SFM and T Cell Expansion Supplement (ThermoFisher Cat. A1048501), 5% human AB serum (GeminiBio, Cat. 100-512) 1× Penicillin-Streptomycin, 1× Glutamax, 10 mM HEPES, 200 U/mL recombinant human interleukin-2 (Peprotech, Cat. 200-02), 5 ng/ml recombinant human interleukin 7 (Peprotech, Cat. 200-07), and 5 ng/ml recombinant human interleukin 15 (Peprotech, Cat. 200-15). After 24 hours in this media. T cells were activated with T Cell TransAct™, human reagent (Miltenyi, Cat. 130-111-160) added at a 1:100 ratio by volume.

### Example 37.2. T Cell LNP Treatment and Expansion

Forty-eight hours post-activation, T cells were harvested, centrifuged at 500 g for 5 min, and resuspended at a concentration of 1×10\hat{ }6 T cells/mL in T cell plating media (TCPM): a serum-free version of TCGM containing 400 U/mL recombinant human interleukin-2 (Peprotech, Cat. 200-02), 10 ng/ml recombinant human interleukin 7 (Peprotech, Cat. 200-07), and 10 ng/ml recombinant human interleukin 15 (Peprotech, Cat. 200-15). Fifty thousand T cells in 50 ul TCPM were added per well to be treated in flat-bottom 96-well plates.

LNPs were generated as described in Example 1 at a molar ratio of 35/47.5/15/2.5 (Lipid A/cholesterol/DSPC/PEG2k-DMG). Prior to T cell treatment, two separate LNP mixes (referred below as mixes “A” and “B”) were prepared in T cell treatment media (TCTM): a version of TCGM containing 20 ug/mL rhApoE3 (Peprotech, Cat. 350-02) in the absence of interleukins 2, 5 or 7.

To determine the minimum levels of BC22n-HiBiT mRNA necessary for saturating total editing, mix “A” consisted of an LNP with BC22n-HiBiT mRNA (SEQ ID NO: 4) diluted to 6.68 μg/mL (3.83 nM), while mix “B” consisted of an LNP with UGI mRNA (SEQ ID NO: 34) diluted to 1.67 μg/mL (5.8 nM) and a multi-cargo LNP containing sgRNAs G023520 (TRAC), G023524 (TRBC1/2), G023521 (CIITA) and G023523 (HLA-A) at the ratio of 4.5/8.7/21.4/65.4 diluted to 6.68 μg/mL (225.19 nM). LNP mixes “A” and “B” were individually incubated at 37° C. for 15 minutes. Mix “A” was serially diluted 1:4 in TCTM, and mixed 1:1 by volume with mix “B”. The resulting solution was added to T cells in 96-well plates at a 1:1 ratio by volume (50 μL per well).

To determine the minimum levels of UGI-HiBiT mRNA necessary for saturating C to T purity, mix “A” consisted of an LNP with UGI-HiBiT mRNA (SEQ ID NO: 316) diluted to 6.68 μg/mL (21.84 nM), while mix “B” consisted of an LNP with BC22n mRNA (SEQ ID NO: 1) diluted to 3.34 μg/mL (1.94 nM) and a multi-cargo LNP containing sgRNAs G023520 (TRAC), G023524 (TRBC1/2), G023521 (CIITA) and G023523 (HLA-A) at the ratio of 4.5/8.7/21.4/65.4 diluted to 6.68 μg/mL (225.19 nM). LNP mixes “A” and “B” were individually incubated at 37° C. for 15 minutes. Mix “A” was serially diluted 1:4 in TCTM, and mixed 1:1 by volume with mix “B”. The resulting solution was added to T cells in 96-well plates at a 1:1 ratio by volume (50 μL per well).

To determine the minimum levels of BC22-2XUGI-HiBiT mRNA necessary for saturating total editing and C to T purity, mix “A” consisted of an LNP with BC22-2XUGI-HiBiT mRNA (SEQ ID NO: 314) diluted to 6.68 μg/mL (3.46 nM), while mix “B” consisted of a multi-cargo LNP containing sgRNAs G023520 (TRAC), G023524 (TRBC1/2), G023521 (CIITA) and G023523 (HLA-A) at the ratio of 4.5/8.7/21.4/65.4 diluted to 6.68 μg/mL (225.19 nM). LNP mixes “A” and “B” were incubated individually at 37° C. for 15 minutes. Mix “A” was serially diluted 1:4 in TCTM, and mixed 1:1 by volume with mix “B”. The resulting solution was added to T cells in 96-well plates at a 1:1 ratio by volume (50 μL per well).

Following the addition of LNPs, T cells were incubated at 37° C. for 24 hours, at which time half of the cells were harvested for cell viability and protein expression assays, and the remaining cells were centrifuged at 500 g for 5 min, resuspended in 200 μL of TCGM and returned to the incubator.

### Example 37.3. Evaluation of Editing Outcomes by Next Generation Sequencing (NGS)

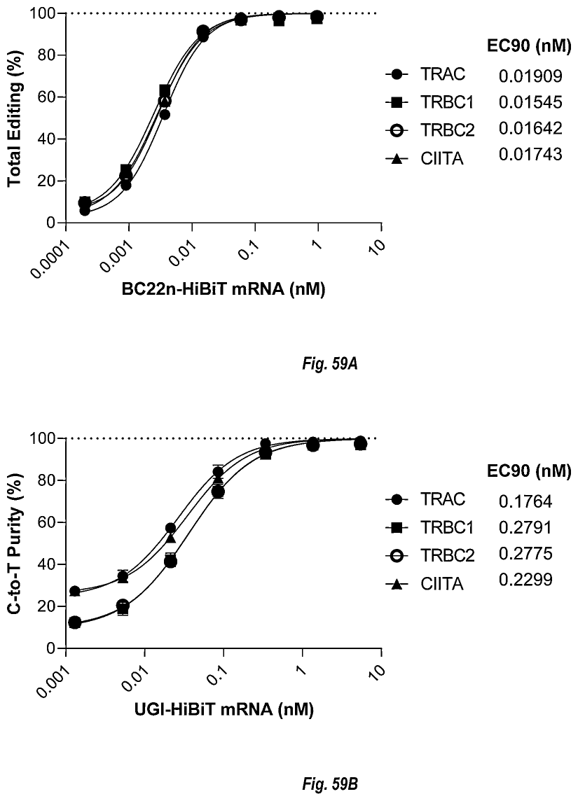

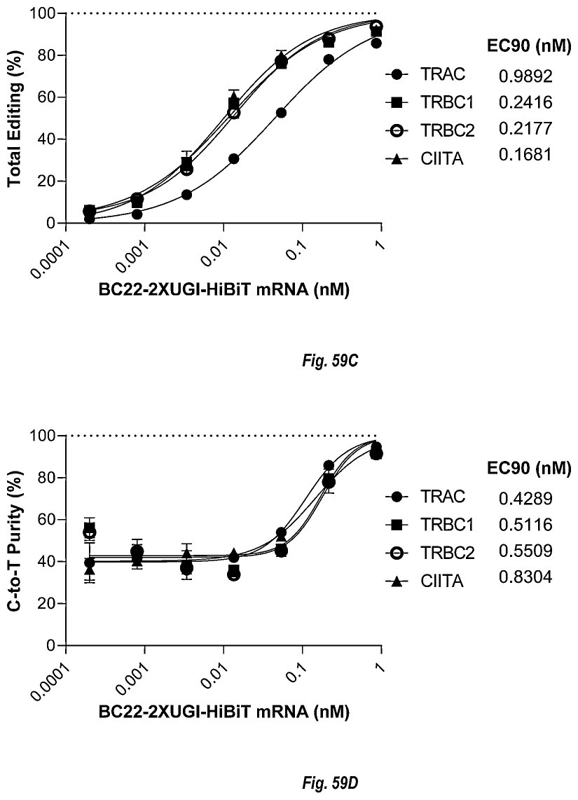

On day 4 post-LNP treatment, T cells were centrifuged at 500 g for 5 min, subjected to lysis, PCR amplification of each targeted locus and subsequent NGS analysis, as described in Example 1. Editing results at the TRAC, TRBC1, TRBC2, and CIITA loci with different concentrations of HiBiT mRNAs are shown in Tables 92-95, respectively. FIG. 59A shows total editing at different BC22n-HiBiT mRNA concentrations. FIG. 59B shows C-to-T purity at different UGI-HiBiT mRNA concentrations. Total editing and C-to-T puity with different BC22-2XUGI-HiBiT mRNA concentrations are shown in FIG. 59C and FIG. 59D, respectively.

Tables 92-95 show editing data for 4 different genomic loci (TRAC, TRBC1, TRBC2 and CIITA).

TABLE 92
Mean editing at TRAC locus in T cells.

| | | | | | | | | | Total | | C-to-T | |
|---|---|---|---|---|---|---|---|---|---|---|---|---|
| | Dose | | C-to-T | | C-to-A/G | | Indels | | editing | | purity | |
| mRNA | (nM) | n | Mean | SD | Mean | SD | Mean | SD | Mean | SD | Mean | SD |
| BC22n- | 0.9584 | 3 | 97.29 | 0.35 | 0.84 | 0.29 | 0.60 | 0.36 | 98.72 | 0.16 | 98.55 | 0.22 |
| HIBIT | 0.2396 | 3 | 97.82 | 0.31 | 0.58 | 0.27 | 0.67 | 0.20 | 99.07 | 0.32 | 98.74 | 0.07 |
| | 0.0599 | 3 | 96.50 | 0.50 | 0.79 | 0.22 | 0.68 | 0.15 | 97.97 | 0.31 | 98.50 | 0.36 |
| | 0.0150 | 3 | 87.53 | 0.41 | 0.68 | 0.09 | 0.45 | 0.31 | 88.66 | 0.14 | 98.72 | 0.39 |
| | 0.0037 | 3 | 50.82 | 2.39 | 0.57 | 0.13 | 0.32 | 0.15 | 51.71 | 2.22 | 98.27 | 0.49 |
| | 0.0009 | 3 | 17.33 | 1.00 | 0.46 | 0.17 | 0.17 | 0.13 | 17.97 | 1.18 | 96.50 | 1.09 |
| | 0.0002 | 3 | 5.31 | 0.28 | 0.37 | 0.10 | 0.19 | 0.07 | 5.88 | 0.21 | 90.36 | 1.67 |
| | 0.0000 | 3 | 0.67 | 0.08 | 0.44 | 0.13 | 0.13 | 0.04 | 1.24 | 0.18 | NA | NA |
| UGI- | 5.4608 | 3 | 97.00 | 0.65 | 0.81 | 0.37 | 0.37 | 0.23 | 98.18 | 0.38 | 98.80 | 0.29 |
| HIBIT | 1.3652 | 1 | 97.22 | ND | 0.28 | ND | 1.39 | ND | 98.89 | ND | 98.31 | ND |
| | 0.3413 | 3 | 95.90 | 0.49 | 1.43 | 0.16 | 1.01 | 0.21 | 98.35 | 0.58 | 97.51 | 0.18 |
| | 0.0853 | 3 | 82.34 | 3.24 | 7.45 | 2.31 | 8.06 | 0.86 | 97.85 | 0.15 | 84.15 | 3.22 |
| | 0.0213 | 3 | 55.53 | 1.88 | 17.35 | 3.45 | 23.98 | 2.08 | 96.86 | 0.39 | 57.34 | 2.17 |
| | 0.0053 | 3 | 32.80 | 2.32 | 24.36 | 3.54 | 38.14 | 1.67 | 95.30 | 1.10 | 34.44 | 2.78 |
| | 0.0013 | 3 | 26.28 | 0.77 | 28.61 | 2.18 | 41.47 | 1.09 | 96.36 | 0.43 | 27.28 | 0.91 |
| | 0.0000 | 3 | 22.46 | 1.08 | 28.38 | 3.67 | 44.56 | 2.27 | 95.40 | 0.37 | 23.55 | 1.22 |
| BC22- | 0.8638 | 3 | 81.31 | 2.75 | 2.41 | 0.03 | 2.10 | 0.85 | 85.82 | 1.90 | 94.73 | 1.12 |
| 2XUGI- | 0.2159 | 3 | 67.03 | 1.79 | 5.49 | 1.44 | 5.52 | 0.11 | 78.03 | 1.36 | 85.90 | 1.92 |
| HIBIT | 0.0540 | 3 | 28.40 | 1.39 | 9.46 | 0.96 | 14.74 | 0.45 | 52.60 | 1.77 | 53.99 | 1.31 |
| | 0.0135 | 3 | 12.89 | 1.33 | 6.69 | 0.29 | 11.11 | 0.84 | 30.69 | 1.91 | 41.93 | 2.10 |
| | 0.0034 | 3 | 4.87 | 0.42 | 2.68 | 0.10 | 5.99 | 0.61 | 13.54 | 0.80 | 35.97 | 1.75 |
| | 0.0008 | 3 | 1.86 | 0.23 | 0.85 | 0.19 | 1.51 | 0.30 | 4.22 | 0.47 | 44.33 | 6.34 |
| | 0.0002 | 3 | 0.82 | 0.21 | 0.48 | 0.01 | 0.77 | 0.18 | 2.07 | 0.04 | 39.42 | 9.51 |
| | 0.0000 | 3 | 0.63 | 0.05 | 0.37 | 0.08 | 0.13 | 0.09 | 1.13 | 0.13 | NA | NA |

TABLE 93
Mean editing at TRBC1 locus in T cells.

| | | | | | | | | | Total | | C-to-T | |
|---|---|---|---|---|---|---|---|---|---|---|---|---|
| | Dose | | C-to-T | | C-to-A/G | | Indels | | editing | | purity | |
| mRNA | (nM) | n | Mean | SD | Mean | SD | Mean | SD | Mean | SD | Mean | SD |
| BC22n- | 0.9584 | 3 | 94.22 | 1.08 | 1.41 | 0.30 | 1.71 | 0.25 | 97.33 | 0.57 | 96.80 | 0.56 |
| HIBIT | 0.2396 | 3 | 93.82 | 0.51 | 1.35 | 0.28 | 1.25 | 0.12 | 96.41 | 0.22 | 97.31 | 0.31 |
| | 0.0599 | 3 | 94.12 | 0.66 | 1.24 | 0.15 | 1.43 | 0.38 | 96.80 | 0.53 | 97.24 | 0.24 |
| | 0.0150 | 3 | 88.80 | 1.47 | 1.30 | 0.28 | 0.86 | 0.19 | 90.96 | 1.38 | 97.62 | 0.14 |
| | 0.0037 | 3 | 61.60 | 1.71 | 1.13 | 0.21 | 0.75 | 0.32 | 63.48 | 2.11 | 97.05 | 0.55 |
| | 0.0009 | 3 | 24.05 | 2.20 | 1.08 | 0.09 | 0.34 | 0.20 | 25.47 | 2.36 | 94.43 | 0.19 |
| | 0.0002 | 3 | 8.54 | 0.72 | 1.11 | 0.08 | 0.30 | 0.05 | 9.95 | 0.71 | 85.79 | 1.62 |
| | 0.0000 | 3 | 2.52 | 0.08 | 1.09 | 0.09 | 0.30 | 0.05 | 3.92 | 0.07 | NA | NA |
| UGI- | 5.4608 | 3 | 93.60 | 1.03 | 1.45 | 0.08 | 1.38 | 0.26 | 96.44 | 0.90 | 97.06 | 0.26 |
| HIBIT | 1.3652 | 3 | 93.90 | 0.77 | 1.59 | 0.16 | 1.57 | 0.49 | 97.05 | 0.33 | 96.75 | 0.47 |
| | 0.3413 | 3 | 89.15 | 1.94 | 4.27 | 1.30 | 2.99 | 0.98 | 96.41 | 0.34 | 92.48 | 2.33 |
| | 0.0853 | 3 | 72.64 | 2.56 | 12.64 | 0.23 | 11.89 | 3.35 | 97.16 | 0.88 | 74.78 | 3.31 |
| | 0.0213 | 3 | 40.67 | 3.56 | 24.32 | 0.89 | 31.25 | 2.94 | 96.25 | 1.16 | 42.23 | 3.22 |
| | 0.0053 | 3 | 17.56 | 3.20 | 32.38 | 1.05 | 42.82 | 2.69 | 92.76 | 1.49 | 18.90 | 3.14 |
| | 0.0013 | 3 | 11.59 | 2.11 | 34.92 | 3.79 | 48.02 | 5.70 | 94.53 | 1.67 | 12.27 | 2.31 |
| | 0.0000 | 3 | 8.51 | 1.36 | 33.48 | 1.78 | 51.60 | 2.15 | 93.60 | 1.31 | 9.08 | 1.34 |
| BC22- | 0.8638 | 3 | 83.49 | 0.86 | 4.26 | 0.27 | 3.64 | 0.71 | 91.39 | 1.58 | 91.36 | 0.93 |
| 2XUGI- | 0.2159 | 3 | 68.57 | 2.32 | 10.23 | 1.51 | 7.39 | 1.54 | 86.19 | 1.60 | 79.54 | 1.23 |
| HIBIT | 0.0540 | 3 | 35.21 | 1.71 | 20.43 | 1.18 | 20.76 | 0.94 | 76.41 | 2.69 | 46.07 | 0.61 |
| | 0.0135 | 3 | 20.21 | 1.22 | 18.44 | 1.47 | 18.28 | 1.63 | 56.92 | 2.62 | 35.56 | 2.76 |
| | 0.0034 | 3 | 10.88 | 0.49 | 9.98 | 1.82 | 8.13 | 3.43 | 28.99 | 5.28 | 38.36 | 6.83 |
| | 0.0008 | 3 | 4.48 | 1.11 | 2.35 | 0.47 | 3.64 | 1.34 | 10.46 | 2.87 | 43.01 | 2.64 |
| | 0.0002 | 3 | 3.39 | 0.37 | 1.71 | 0.23 | 0.97 | 0.69 | 6.07 | 0.83 | 56.10 | 4.73 |
| | 0.0000 | 3 | 2.81 | 0.22 | 1.15 | 0.09 | 0.34 | 0.07 | 4.30 | 0.26 | NA | NA |

TABLE 94
Mean editing at TRBC2 locus in T cells

| | | | | | | | | | Total | | C-to-T | |
|---|---|---|---|---|---|---|---|---|---|---|---|---|
| | Dose | | C-to-T | | C-to-A/G | | Indels | | editing | | purity | |
| mRNA | (nM) | n | Mean | SD | Mean | SD | Mean | SD | Mean | SD | Mean | SD |
| BC22n- | 0.9584 | 3 | 94.58 | 0.49 | 1.89 | 0.43 | 1.90 | 0.15 | 98.37 | 0.20 | 96.14 | 0.31 |
| HIBIT | 0.2396 | 3 | 95.25 | 0.63 | 1.68 | 0.04 | 1.20 | 0.26 | 98.13 | 0.42 | 97.06 | 0.26 |
| | 0.0599 | 3 | 94.01 | 0.63 | 1.36 | 0.09 | 1.63 | 0.44 | 97.00 | 0.58 | 96.92 | 0.37 |
| | 0.0150 | 3 | 88.99 | 0.35 | 1.39 | 0.27 | 1.04 | 0.19 | 91.41 | 0.08 | 97.35 | 0.45 |
| | 0.0037 | 3 | 56.12 | 2.38 | 1.29 | 0.12 | 0.74 | 0.30 | 58.15 | 2.64 | 96.51 | 0.61 |
| | 0.0009 | 3 | 20.90 | 0.73 | 1.20 | 0.25 | 0.40 | 0.09 | 22.50 | 0.59 | 92.85 | 1.31 |
| | 0.0002 | 3 | 8.08 | 1.04 | 1.11 | 0.15 | 0.32 | 0.09 | 9.51 | 0.94 | 84.81 | 3.36 |
| | 0.0000 | 3 | 2.97 | 0.24 | 1.08 | 0.10 | 0.49 | 0.24 | 4.55 | 0.51 | NA | NA |
| UGI- | 5.4608 | 3 | 94.87 | 0.14 | 1.43 | 0.21 | 1.06 | 0.20 | 97.35 | 0.12 | 97.45 | 0.02 |
| HIBIT | 1.3652 | 1 | 94.76 | 0.11 | 1.67 | 0.13 | 1.51 | 0.09 | 97.94 | 0.14 | 96.76 | 0.20 |
| | 0.3413 | 3 | 91.81 | 0.45 | 3.34 | 0.40 | 3.16 | 0.47 | 98.31 | 0.20 | 93.39 | 0.27 |
| | 0.0853 | 3 | 73.09 | 0.73 | 12.14 | 0.96 | 12.56 | 0.15 | 97.79 | 0.41 | 74.74 | 0.95 |
| | 0.0213 | 3 | 39.77 | 0.07 | 23.73 | 1.69 | 32.65 | 1.19 | 96.15 | 1.22 | 41.37 | 0.53 |
| | 0.0053 | 3 | 19.66 | 0.29 | 29.74 | 0.81 | 46.54 | 0.63 | 95.93 | 0.45 | 20.49 | 0.20 |
| | 0.0013 | 3 | 11.79 | 1.55 | 31.00 | 1.01 | 52.42 | 3.12 | 95.21 | 0.94 | 12.40 | 1.73 |
| | 0.0000 | 3 | 9.04 | 1.42 | 32.73 | 1.37 | 53.94 | 1.88 | 95.71 | 1.35 | 9.45 | 1.57 |
| BC22- | 0.8638 | 3 | 85.85 | 2.78 | 4.34 | 0.88 | 3.57 | 0.69 | 93.76 | 1.23 | 91.55 | 1.76 |
| 2XUGI- | 0.2159 | 3 | 68.48 | 2.35 | 10.64 | 0.47 | 8.73 | 0.23 | 87.85 | 1.95 | 77.93 | 0.95 |
| HIBIT | 0.0540 | 3 | 34.87 | 2.26 | 19.85 | 1.23 | 22.41 | 1.15 | 77.12 | 0.42 | 45.21 | 2.75 |
| | 0.0135 | 3 | 17.86 | 0.57 | 16.46 | 0.44 | 18.42 | 1.68 | 52.74 | 2.57 | 33.89 | 0.95 |
| | 0.0034 | 3 | 9.52 | 1.12 | 8.01 | 0.14 | 8.16 | 0.27 | 25.69 | 1.10 | 37.01 | 2.90 |
| | 0.0008 | 3 | 5.17 | 0.59 | 3.15 | 0.38 | 3.19 | 0.19 | 11.50 | 0.65 | 44.83 | 3.28 |
| | 0.0002 | 3 | 3.06 | 0.16 | 1.38 | 0.14 | 1.26 | 0.35 | 5.71 | 0.54 | 53.91 | 3.87 |
| | 0.0000 | 3 | 2.79 | 0.05 | 1.11 | 0.22 | 0.35 | 0.12 | 4.25 | 0.28 | NA | NA |

TABLE 95
Mean editing at CIITA locus in T cells

| | | | | | | | | | Total | | C-to-T | |
|---|---|---|---|---|---|---|---|---|---|---|---|---|
| | Dose | | C-to-T | | C-to-A/G | | Indels | | editing | | purity | |
| mRNA | (nM) | n | Mean | SD | Mean | SD | Mean | SD | Mean | SD | Mean | SD |
| BC22n- | 0.9584 | 3 | 96.75 | 0.28 | 1.68 | 0.18 | 0.57 | 0.07 | 99.00 | 0.05 | 97.72 | 0.23 |
| HIBIT | 0.2396 | 3 | 96.80 | 0.15 | 1.51 | 0.25 | 0.53 | 0.11 | 98.83 | 0.24 | 97.94 | 0.14 |
| | 0.0599 | 3 | 96.31 | 0.64 | 1.39 | 0.17 | 0.42 | 0.17 | 98.12 | 0.43 | 98.15 | 0.31 |
| | 0.0150 | 3 | 89.04 | 1.09 | 1.19 | 0.07 | 0.41 | 0.07 | 90.63 | 0.99 | 98.24 | 0.16 |
| | 0.0037 | 3 | 56.77 | 1.94 | 1.24 | 0.18 | 0.18 | 0.02 | 58.18 | 1.79 | 97.56 | 0.38 |
| | 0.0009 | 3 | 22.38 | 0.53 | 1.29 | 0.07 | 0.12 | 0.07 | 23.78 | 0.60 | 94.09 | 0.13 |
| | 0.0002 | 3 | 7.16 | 0.27 | 1.19 | 0.14 | 0.16 | 0.05 | 8.52 | 0.35 | 84.06 | 1.27 |
| | 0.0000 | 3 | 0.88 | 0.06 | 1.16 | 0.27 | 0.05 | 0.02 | 2.09 | 0.22 | NA | NA |
| UGI- | 5.4608 | 3 | 96.10 | 0.31 | 1.49 | 0.20 | 0.45 | 0.29 | 98.04 | 0.13 | 98.02 | 0.37 |
| HIBIT | 1.3652 | 1 | 96.09 | 0.59 | 1.69 | 0.25 | 0.92 | 0.25 | 98.69 | 0.13 | 97.36 | 0.51 |
| | 0.3413 | 3 | 94.04 | 0.58 | 3.10 | 0.29 | 1.70 | 0.58 | 98.83 | 0.12 | 95.15 | 0.65 |
| | 0.0853 | 3 | 80.13 | 1.16 | 8.99 | 0.79 | 9.57 | 0.60 | 98.69 | 0.06 | 81.20 | 1.17 |
| | 0.0213 | 3 | 51.75 | 1.05 | 20.78 | 0.75 | 25.48 | 0.97 | 98.02 | 0.40 | 52.80 | 1.28 |
| | 0.0053 | 3 | 32.69 | 1.17 | 27.04 | 1.89 | 37.48 | 0.98 | 97.21 | 0.34 | 33.63 | 1.14 |
| | 0.0013 | 3 | 26.59 | 0.34 | 26.78 | 1.72 | 43.51 | 1.10 | 96.88 | 1.06 | 27.45 | 0.37 |
| | 0.0000 | 3 | 25.47 | 1.76 | 27.78 | 1.86 | 43.47 | 3.32 | 96.72 | 0.63 | 26.33 | 1.72 |
| BC22- | 0.8638 | 3 | 86.31 | 2.68 | 3.78 | 1.26 | 2.13 | 0.51 | 92.22 | 1.69 | 93.59 | 1.81 |
| 2XUGI- | 0.2159 | 3 | 67.79 | 6.14 | 9.39 | 1.85 | 9.25 | 3.55 | 86.43 | 1.80 | 78.37 | 5.68 |
| HIBIT | 0.0540 | 3 | 41.54 | 1.66 | 17.05 | 0.26 | 21.16 | 1.07 | 79.75 | 2.54 | 52.08 | 0.85 |
| | 0.0135 | 3 | 26.63 | 0.45 | 14.36 | 1.00 | 19.33 | 2.27 | 60.32 | 3.17 | 44.23 | 2.37 |
| | 0.0034 | 3 | 12.13 | 1.48 | 6.22 | 1.17 | 9.19 | 0.82 | 27.54 | 2.50 | 44.09 | 4.45 |
| | 0.0008 | 3 | 3.99 | 0.46 | 2.94 | 0.39 | 2.94 | 0.45 | 9.87 | 1.00 | 40.47 | 3.90 |
| | 0.0002 | 3 | 1.40 | 0.38 | 1.56 | 0.34 | 0.83 | 0.23 | 3.79 | 0.51 | 36.41 | 5.18 |
| | 0.0000 | 3 | 0.67 | 0.10 | 1.10 | 0.16 | 0.09 | 0.08 | 1.86 | 0.17 | NA | NA |

### Example 37.4. Evaluation of Intracellular Protein Levels

Twenty-four hours post-LNP treatments, all samples were mixed thoroughly and two aliquots of 25 JAL were transferred to white-walled 96-well plates containing 75 μL of TCGM. One plate was subjected to a CellTiter-Glo® 2.0 Cell Viability Assay (Promega Cat No. G9242) and the other plate was subjected to a Nano-Glo® HiBiT Lytic Detection Assay (Promega Cat No. N3040). Both assays were performed following the manufacturer's protocol. Standards curves were prepared using known numbers of donor-matched T cells or a commercially available protein with a HiBiT tag (Promega Cat No. N3010). HiBiT quantitation is presented in Table % normalized to the expression level of the 0.0037 nM BC22n-HiBiT mRNA sample.

TABLE 96
Mean protein expression (arbitrary units) for HiBiT-tagged proteins.

| | Dose | | | |
|---|---|---|---|---|
| mRNA | (nM) | n | Average | SD |
| BC22n-HiBiT | 0.9584 | 3 | 51.93 | 2.20 |
| | 0.2396 | 3 | 33.39 | 4.42 |
| | 0.0599 | 3 | 15.40 | 1.34 |
| | 0.0150 | 3 | 4.05 | 0.07 |
| | 0.0037 | 3 | 1.00 | 0.05 |
| UGI-HIBIT | 5.4608 | 3 | 1499.52 | 106.66 |
| | 1.3652 | 3 | 583.54 | 53.95 |
| | 0.3413 | 3 | 180.50 | 16.40 |
| | 0.0853 | 3 | 55.38 | 3.55 |
| | 0.0213 | 3 | 16.05 | 1.26 |
| | 0.0053 | 3 | 4.32 | 0.32 |
| BC22-2XUGI-HIBIT | 0.8638 | 3 | 80.23 | 4.45 |
| | 0.2159 | 3 | 43.95 | 10.80 |
| | 0.0540 | 3 | 14.74 | 1.31 |
| | 0.0135 | 3 | 4.17 | 0.66 |
| | 0.0034 | 3 | 1.32 | 0.15 |

Data from Table 96 was used to generate hyperbolic curves and interpolate the the protein units expressed at the minimum doses required to achieve saturating editing or C-to-T purity levels. The minimum dose as saturating levels is defined here as twice the EC90 (2×EC90), the concentration required to achieve 90% total editing or C-to-T purity, respectively, for each mRNA tested.

Table 97 shows the 2×EC90 doses of the mRNAs tested in this experiment and the BC22 and UGI peptide units and their ratios at these doses. For base editors and UGI-HiBiT, protein units are equal to peptide units. UGI peptide units for BC22-2XUGI were calculated by multiplying the BC22-2XUGI protein units by a factor of 2. Table 98 shows the ratio of base editor peptide units at total editing 2×EC90 to the UGI peptide units at C to T purity 2×EC90.

TABLE 97
2X EC90 for mRNA and protein at relevant editing readouts.

| | Editing | | | Peptide |
|---|---|---|---|---|
| HiBiT mRNA | Readout | Locus | nM | units |
| BC22n-HiBiT | Total Editing | TRAC | 0.038 | 10.07 |
| | | TRBC1 | 0.031 | 8.41 |
| | | TRBC2 | 0.033 | 8.86 |
| | | CIITA | 0.035 | 9.32 |
| | | Average | 0.034 | 9.16 |
| | | SD | 0.003 | 0.71 |
| UGI-HIBIT | C-to-T purity | TRAC | 0.354 | 177.22 |
| BC22-2XUGI-HiBIT | | TRBC1 | 0.560 | 271.33 |
| | | TRBC2 | 0.557 | 269.91 |
| | | CIITA | 0.461 | 227.02 |
| | | Average | 0.483 | 236.37 |
| | | SD | 0.098 | 44.48 |
| | Total Editing | TRAC | 1.979 | 95.56 |
| | C-to-T purity | TRBC1 | 0.480 | 65.60 |
| | | TRBC2 | 0.434 | 62.84 |
| | | CIITA | 0.334 | 55.54 |
| | | Average | 0.807 | 69.88 |
| | | SD | 0.784 | 17.63 |
| | | TRAC | 0.859 | 160.35 |
| | | TRBC1 | 1.024 | 168.00 |
| | | TRBC2 | 1.103 | 171.04 |
| | | CIITA | 1.660 | 185.66 |
| | | Average | 1.161 | 171.26 |
| | | SD | 0.348 | 10.60 |

TABLE 98
Base editor to UGI enzyme ratios.

| | | Base Editor-to-UGI |
|---|---|---|
| Constructs | Locus | enzyme ratio |
| BC22n-HiBIT / UGI- | TRAC | 1 to 18 |
| HIBIT | TRBC1 | 1 to 32 |
| | TRBC2 | 1 to 30 |
| | CIITA | 1 to 24 |
| | Average | 1 to 26 |
| | SD | 7 |
| BC22-2XUGI-HIBIT | TRAC | 1 to 1.7 |
| | TRBC1 | 1 to 2.6 |
| | TRBC2 | 1 to 2.7 |
| | CIITA | 1 to 3.3 |
| | Average | 1 to 2.5 |
| | SD | 0.6 |

### Example 38. In Vivo Editing with UGI in Trans

In vivo editing profiles of deaminase containing constructs were compared to Cas9 when UGI was delivered in trans (as a separate mRNA). The constructs used encoded a fusion protein including D10A Cas9 with a deaminase.

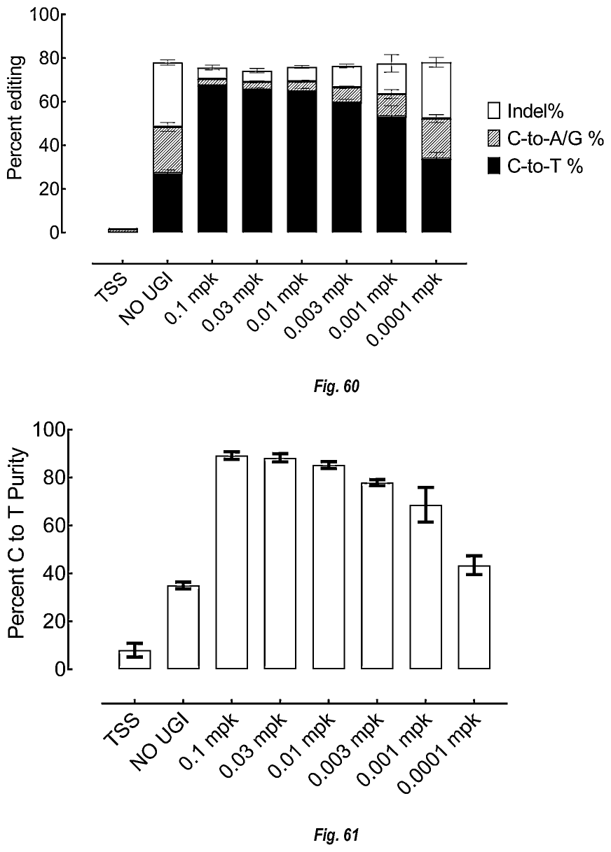

Twenty-four commercially available CD-1 female mice ranging from 6-10 weeks of age (n=3 per group) were used in this study. Animals were weighed pre-dose for dosing calculations. Each RNA species was formulated separately in an LNP. Formulations containing editor mRNA, UGI mRNA and G019427 sgRNA were mixed in a w/w ratio of RNA cargos. The formulation mixture for Group 2 contained only editor mRNA and sgRNA and these were mixed in a w/w ratio of 2:1 (editor mRNA:sgRNA). Groups 3-8 contained mRNA:sgRNA at a w/w ratio of 2:1, and UGI mRNA mixed in at w/w ratios of 1:3, 1:10, 1:30, 1:100, 1:300, and 1:3000 (editor mRNA+sgRNA:UGI mRNA). Apart from the negative control group, which was dosed with TSS buffer only, all groups resulted in editing at the ANACP5 locus targeted with G019427. Formulations were administered intravenously via tail vein injection according to the doses listed in Table 99. Animals were periodically observed for adverse effects for at least 24 hours post-dose. Six days after treatment, animals were euthanized by cardiac puncture under isoflurane anesthesia; liver tissue were collected for downstream analysis. Liver punches weighing between 5 and 15 mg were collected for isolation of genomic DNA. Genomic DNA samples were analyzed with NGS sequencing as described in Example 1. Editing data are shown in Table 100 and FIG. 60. FIG. 60 shows C to T purity.

TABLE 99
Dosage of RNA species for each experimental group

| | | | Editor | sgRNA | UGI |
|---|---|---|---|---|---|
| | | | mRNA | dose | mRNA |
| Group | Samples | LNP cargos | (mg/kg) | (mg/kg) | (mg/kg) |
| 1 | TSS | None | 0 | 0 | 0 |
| 2 | 0.0 MPK UGI | BC22n (SEQ ID NO: 2) | 0.2 | 0.1 | 0.0 |
| 3 | 0.1 MPK UGI | BC22n (SEQ ID NO: 2) | | | 0.1 |
| 4 | 0.03 MPK UGI | G019427 | | | 0.03 |
| 5 | 0.01 MPK UGI | UGI (SEQ ID NO: 26) | | | 0.01 |
| 6 | 0.003 MPK UGI | | | | 0.003 |
| 7 | 0.001 MPK UGI | | | | 0.001 |
| 8 | 0.0001 MPK UGI | | | | 0.0001 |

TABLE 100
Mean editing in mouse livers

| | C-to-T | | C-to-A/G | | Indel | | C to T Purity | |
|---|---|---|---|---|---|---|---|---|
| Group | Mean | SD | Mean | SD | Mean | SD | Mean | SD |
| 1 | 0.13 | 0.06 | 1.42 | 0.07 | 0.09 | 0.02 | 7.99 | 2.88 |
| 2 | 27.30 | 1.40 | 21.13 | 1.99 | 29.55 | 1.16 | 35.00 | 1.39 |
| 3 | 67.47 | 0.35 | 2.99 | 0.31 | 5.19 | 1.05 | 89.21 | 1.57 |
| 4 | 65.50 | 0.87 | 3.56 | 0.40 | 5.17 | 1.01 | 88.25 | 1.68 |
| 5 | 64.73 | 1.37 | 4.54 | 0.49 | 6.65 | 0.64 | 85.25 | 1.39 |
| 6 | 59.53 | 1.50 | 7.07 | 0.52 | 9.79 | 0.84 | 77.93 | 1.24 |
| 7 | 53.20 | 4.94 | 10.27 | 1.99 | 14.08 | 4.01 | 68.67 | 7.23 |
| 8 | 33.90 | 2.88 | 18.37 | 1.82 | 25.81 | 2.21 | 43.44 | 3.90 |

### Example 39. Cytotoxic Susceptibility of Engineered T Cells

Engineered T cells were assayed for cytotoxic susceptibility when targeted by natural killer (NK) cells.

NK cells (Stemcell Technologies) were thawed and resuspended at a cell concentration of 1×10\hat{ }6 cells/ml into T cell growth media (TCGM) composed of OpTmizer TCGM and further supplemented with 100 U/mL of recombinant human interleukin-2 (Peprotech, Cat. 200-02), 5 ng/mL IL-7 (Peprotech, Cat. 200-07), 5 ng/mL IL-15 (Peprotech, Cat. 200-15). Cells were incubated at 37° C. for 24 hours.

Twenty-four hours post thaw, the NK cells were labelled with 0.5 μM Cell Trace Violet (CTV) as follows: a vial of CTV (CellTrace™ Violet Cell Proliferation Kit, for flow cytometry, Cat. C34571) was reconstituted in DMSO from the kit to give a 5 mM stock concentration. Two μL of CTV stock was diluted with 18 μL Phosphate-Buffered Saline (PBS) (Corning, Cat. 21-040-CV) to obtain a concentration of 0.5 mM. NK cells were centrifuged at 500×g for 5 minutes, the media was aspirated, and cells were resuspended in PBS at a concentration of 1×10\hat{ }6 cells/mL such that the final concentration of CTV dye was 0.5 μM. The cells were mixed with CTV dye solution incubated at 37° C. for 20 minutes. Unbound dye was quenched by the addition of TCGM and incubated for 5 minutes. The cells were centrifuged at 500×g for 5 minutes. Cells are resuspended in TCGM supplemented with 100 U/mL of recombinant human interleukin-2 (Peprotech, Cat. 200-02), 5 ng/mL IL-7 (Peprotech, Cat. 200-07), 5 ng/mL IL-15 (Peprotech, Cat. 200-15) at a concentration of 2×10\hat{ }6 cells/mL. To test a range of effector:target (E:T) ratios, CTV-labelled NK cells were aliquoted in 100 μL of media in a 6-point, 2-fold serial dilution with the highest number of cells being 2×10\hat{ }5 cells. Media-only samples were included as negative controls.

T cells were engineered using BC22n and UGI mRNA as described in Example 14 using G023523 (SEQ ID NO: 501) targeting HLA-A as a test sample and with G023519 (SEQ ID NO: 498) targeting B2M was as a positive control for NK killing. Unedited T cells were assayed as a negative control for NK killing. Other controls for flow cytometry included CTV-labelled NK cells without T cells; a “unstained” sample combining unlabelled NK cells and T cells; and a 1:1 mix of unlabeled heat killed and non-heat killed NK cells and T cells stained with 7AAD. T cells were resuspended at a density of 2×10\hat{ }5 cells in TCGM composed of OpTmizer TCGM and further supplemented with 100 U/mL of recombinant human interleukin-2 (Peprotech, Cat. 200-02), 5 ng/mL IL-7 (Peprotech, Cat. 200-07), and 5 ng/mL IL-15 (Peprotech, Cat. 200-15). Twenty thousand T cells were added to each well of NK cells and media controls. Cells were incubated at 37° C. for 24 hours.

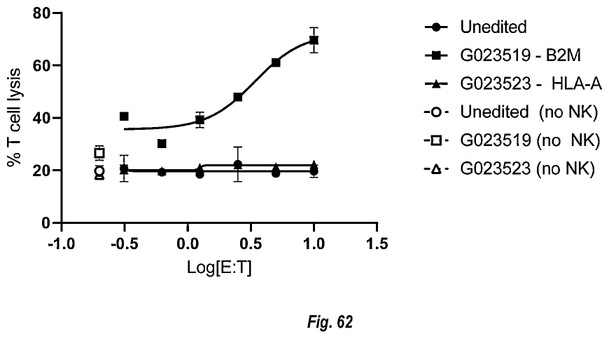

At 24 hours, half of the volume of the cells from the LD heat killed well were heat killed and transferred back to the same well in the assay plate. Cells were centrifuged and resuspended in 80 μL of a 1:200 v/v solution of 7-AAD (BD Biosciences, Cat. 559925) in FACS buffer (PBS+2% FBS (Gibco, Cat. A31605-02)+2 mM EDTA (Invitrogen, Cat. 15-575-020)). Data for specific lysis of T cells were acquired by flow cytometry using a Cytoflex LX instrument (Beckman Coulter) and analyzed using the FlowJo software package. Gates were first drawn on the CTV negative population to gate out the NK cells, followed by gating on singlets after which a gate was drawn on the 7-AAD negative population to gate for the live T cells. The percent lysis of T cells was calculated by subtracting the live cell percentage from 100. T cells edited using BC22n and HLA-A guide G023523 (SEQ ID NO: 501) were protected from NK cell mediated cytotoxicity as shown in Table 101 and FIG. 62.

TABLE 101
Mean percentage lysis of engineered T cells exposed
to HLA-B and C matched NK cells

| | Unedited | | | G023519 B2M | | | G023523 HLA-A | | |
|---|---|---|---|---|---|---|---|---|---|
| E:T | Mean | SD | n | Mean | SD | n | Mean | SD | n |
| 10 | 19.65 | 2.33 | 2 | 69.60 | 4.81 | 2 | 22.23 | 1.10 | 3 |
| 5 | 18.80 | 1.59 | 3 | 61.10 | 0.85 | 2 | 21.35 | 0.49 | 2 |
| 2.5 | 22.27 | 6.62 | 3 | 47.95 | 0.49 | 2 | 22.10 | 1.27 | 2 |
| 1.25 | 18.47 | 1.27 | 3 | 39.20 | 2.98 | 3 | 21.00 | 0.81 | 3 |
| 0.63 | 19.30 | 0.66 | 3 | 30.20 | NA | 1 | 19.75 | 0.35 | 2 |
| 0.31 | 20.70 | 5.02 | 3 | 40.60 | NA | 1 | 20.27 | 1.67 | 3 |
| 0 | 19.77 | 2.01 | 3 | 26.57 | 2.73 | 3 | 18.30 | 1.41 | 3 |

### Example 40—Editing Window for SpyCas9 and Nme2Cas9 Base Editors

The range of guide positions available for deamination by base editors designed with Nme2Cas9 nickase or SpyCas9 nickase and APOBEC3a was assayed by evaluating C to T conversion efficacy on a position by position basis across a panel of target sites with cytosine residues in the guide region.

### Example 40.1 T Cell Preparation

Healthy human donor apheresis was obtained commercially (Hemacare), and cells were washed and resuspended in in CliniMACS® PBS/EDTA buffer (Miltenyi Biotec Cat. 130-070-525) and processed in a MultiMACS™ Cell 24 Separator Plus device (Miltenyi Biotec). T cells were isolated via positive selection using a Straight from Leukopak® CD4/CD8 MicroBead kit, human (Miltenyi Biotec Cat. 130-122-352). T cells were aliquoted and cryopreserved for future use in Cryostor® CS10 (StemCell Technologies Cat. 07930).

Upon thaw, T cells were plated at a density of 1.0×10\hat{ }6 cells/mL in T cell growth media (TCGM) composed of CTS OpTmizer T Cell Expansion SFM and T Cell Expansion Supplement (ThermoFisher Cat. A1048501), 5% human AB serum (GeminiBio, Cat. 100-512) 1× Penicillin-Streptomycin, 1× Glutamax, 10 mM HEPES, 200 U/mL recombinant human interleukin-2 (Peprotech, Cat. 200-02), 5 ng/mL recombinant human interleukin 7 (Peprotech, Cat. 200-07), and 5 ng/ml recombinant human interleukin 15 (Peprotech, Cat. 200-15). T cells were rested in this media for 24 hours, at which time they were activated with T Cell TransAct™, human reagent (Miltenyi, Cat. 130-111-160) added at a ratio of 1:100 by volume. T cells were activated for 48 hours prior to electroporation.

### Example 40.2 T Cell Editing with RNA Electroporation

Solutions containing mRNA encoding Spy BC22n (SEQ ID NO: 1) or Nme2 BC22n (SEQ ID NO: 315) and UGI (SEQ ID NO: 34) were prepared in P3 buffer. Guide RNAs targeting either the SCAP, LINC01588, LSP1, SEC61B, VEGFA, FancF, AAVS1, or ARHGEF9 locus was removed from the storage and denatured for 2 minutes at 95° C. and incubated at room temperature for 5 minutes.

Forty-eight hours post activation, T cells were harvested, centrifuged, and resuspended at a concentration of 12.5×10^{\hat{ }6} T cells/mL in P3 electroporation buffer (Lonza). For each well to be electroporated, 1×10\hat{ }⁵ T cells were mixed with 200 ng of BC22n or Nme2 base editor mRNA, 200 ng of UGI mRNA and 4 μM of sgRNAs in a final volume of 20 μL of P3 electroporation buffer. This mix was transferred in duplicate to 96-well Nucleofector™ plates and electroporated using the manufacturer's pulse code. Electroporated T cells were immediately rested in 80 μL of CTS Optimizer T cell growth media without cytokines for 15 minutes before being transferred to new flat-bottom 96-well plates containing an additional 80 μL of CTS OpTmizer T cell growth media supplemented with 2× cytokines. The resulting plates were incubated at 37° C. for 4 days. On day 4 post-electroporation, 100 μL of cells was harvested for DNA extraction. DNA samples were subjected to PCR and subsequent NGS analysis with two separate primer sets (technical replicates), as described in Example 1.

### Example 40.3 Base Editor Editing Window Analysis

Position values were assigned to each DNA base within the protospacer region and its 5′ and 3′ adjacent nucleotides. or SpyCas9, Position 1-20 represent the 20 bases bound by a SpyCas9 guide wherein position 1 is the PAM distal base and position 20 is the PAM proximal base. For Nme2Cas9, positions 1-24 represent the 24 bases bound by a Nme2Cas9 guide wherein position 1 is the PAM distal base and position 24 is the PAM proximal base. The 5′ and 3′ ends outside of protospacer region were assigned to be negative and positive positions, respectively; relative to Position 1.

Define conversion frequency: A guide can be designed to target the reference strand (strand +) or reverse complementary strand (strand −) of a gene. For strand + guides, each wild-type cytosine position in the target region was recorded and conversion frequency (cytosine to thymine) at the position was calculated as ratio of sequencing reads with thymine at the position versus reads with either cytosine or thymine at the position; For strand-guide, each wild-type guanine position in target region was recorded and a conversion frequency (guanine to adenine) at the position is calculated as ratio of sequencing reads with adenine at the position versus reads with adenine and guanine at the position.

Classify guide activity: Four replicates were measured for each guide: two technical replicates for each of two biological replicates. Only positions in a replicate with >500 sequencing reads would be further analysed. For each guide, the mean conversion frequency of a position is the average of the replicates, and the highest mean conversion frequency of all positions for a guide is used to classify this guide's conversion activity: Low, medium, and high activity guides have a highest mean conversion frequency of <50%, 50% & <70%, >70%, respectively. High activity guides for BC22n (n=38) and Nme2 base editor (n=14) were chosen for further window analysis as shown in Table 102.

TABLE 102
High activity guide sequences included in editing window analysis

| | Editor | | Editor | | Editor |
|---|---|---|---|---|---|
| guide | species | guide | species | guide | species |
| G021514 | Nme2 | G009272 | Spy | G021549 | Spy |
| G021519 | Nme2 | G009295 | Spy | G021573 | Spy |
| G021524 | Nme2 | G009303 | Spy | G021574 | Spy |
| G021529 | Nme2 | G009330 | Spy | G021575 | Spy |
| G021550 | Nme2 | G009333 | Spy | G021576 | Spy |
| G021551 | Nme2 | G012243 | Spy | G021577 | Spy |
| G021552 | Nme2 | G013549 | Spy | G021578 | Spy |
| G021553 | Nme2 | G013578 | Spy | G021579 | Spy |
| G021554 | Nme2 | G021537 | Spy | G021582 | Spy |
| G021555 | Nme2 | G021538 | Spy | G021584 | Spy |
| G021556 | Nme2 | G021540 | Spy | G021585 | Spy |
| G021560 | Nme2 | G021541 | Spy | G021586 | Spy |
| G021562 | Nme2 | G021542 | Spy | G021587 | Spy |
| G021570 | Nme2 | G021543 | Spy | G021588 | Spy |
| G009252 | Spy | G021544 | Spy | G021589 | Spy |
| G009255 | Spy | G021545 | Spy | G021591 | Spy |
| G009260 | Spy | G021548 | Spy | G021593 | Spy |
| G009269 | Spy | | | | |

Define conversion rate for each position across all guides: If a guide has the position recorded, each of its replicates with >500 sequencing reads at the position is considered as a “case”. Total cases were summed for each position of all guides and served as a denominator for conversion rate calculation. In each case, if the conversion frequency is greater than 50%, it is considered as a high conversion “event”, and total events is the sum of high conversion events for each position across all guides and replicates. The “events” serve as numerator for conversion rate calculation. Conversion Rate of a position equals the percentage of high conversion events in all cases for each position.

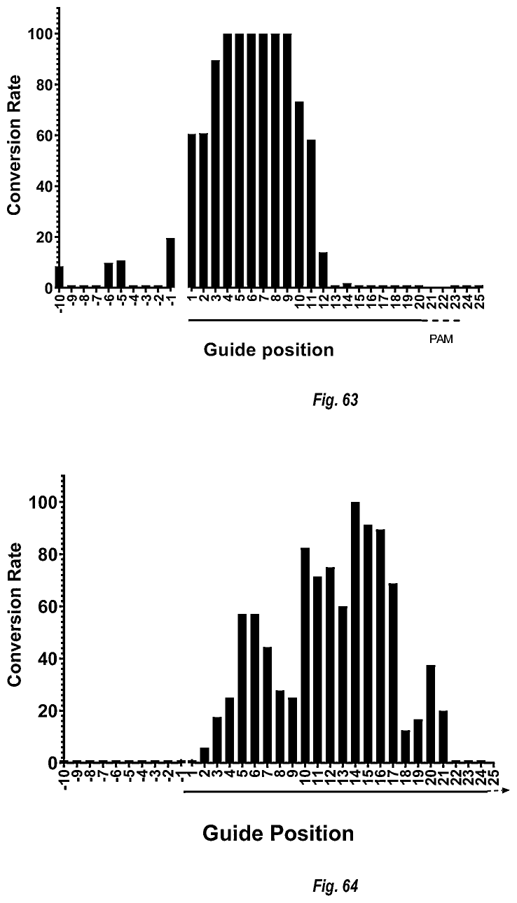

Table 103 displays the position, cases, events, and conversion rate for the spyBC22n C-to-T editing window. FIG. 63 shows conversion rate by position BC22n. FIG. 64 shows conversion rate by position for Nme2 base editor. Positions 1-11 for spyBC22n and positions 4-20 for Nme2BC22 show conversion rates over 25%.

TABLE 103
Conversion rate for Spy base editor and Nme2 base editor constructs.

| | | | | Conversion | | | | | |
|---|---|---|---|---|---|---|---|---|---|
| Editor | Pos. | Events | Cases | Rate | Editor | Pos. | Events | Cases | Conversion Rate |
| Nme2 | −20 | 0 | 14 | 0.0% | Spy | −20 | 0 | 48 | 0.0% |
| Nme2 | −19 | 0 | 9 | 0.0% | Spy | −19 | 0 | 29 | 0.0% |
| Nme2 | −18 | 0 | 24 | 0.0% | Spy | −18 | 0 | 26 | 0.0% |
| Nme2 | −17 | 0 | 24 | 0.0% | Spy | −17 | 0 | 32 | 0.0% |
| Nme2 | −16 | 0 | 24 | 0.0% | Spy | −16 | 0 | 30 | 0.0% |
| Nme2 | −15 | 0 | 14 | 0.0% | Spy | −15 | 0 | 43 | 0.0% |
| Nme2 | −14 | 0 | 15 | 0.0% | Spy | −14 | 0 | 39 | 0.0% |
| Nme2 | −13 | 0 | 9 | 0.0% | Spy | −13 | 0 | 54 | 0.0% |
| Nme2 | −12 | 0 | 17 | 0.0% | Spy | −12 | 0 | 60 | 0.0% |
| Nme2 | −11 | 0 | 17 | 0.0% | Spy | −11 | 0 | 35 | 0.0% |
| Nme2 | −10 | 0 | 10 | 0.0% | Spy | −10 | 4 | 47 | 8.5% |
| Nme2 | −9 | 0 | 24 | 0.0% | Spy | −9 | 0 | 38 | 0.0% |
| Nme2 | −8 | 0 | 12 | 0.0% | Spy | −8 | 0 | 33 | 0.0% |
| Nme2 | −7 | 0 | 14 | 0.0% | Spy | −7 | 0 | 24 | 0.0% |
| Nme2 | −6 | 0 | 30 | 0.0% | Spy | −6 | 4 | 41 | 9.8% |
| Nme2 | −5 | 0 | 19 | 0.0% | Spy | −5 | 4 | 37 | 10.8% |
| Nme2 | −4 | 0 | 12 | 0.0% | Spy | −4 | 0 | 25 | 0.0% |
| Nme2 | −3 | 0 | 4 | 0.0% | Spy | −3 | 0 | 42 | 0.0% |
| Nme2 | −2 | 0 | 24 | 0.0% | Spy | −2 | 0 | 38 | 0.0% |
| Nme2 | −1 | 0 | 18 | 0.0% | Spy | −1 | 11 | 56 | 19.6% |
| Nme2 | 1 | 0 | 14 | 0.0% | Spy | 1 | 26 | 43 | 60.5% |
| Nme2 | 2 | 1 | 17 | 5.9% | Spy | 2 | 31 | 51 | 60.8% |
| Nme2 | 3 | 3 | 17 | 17.6% | Spy | 3 | 34 | 38 | 89.5% |
| Nme2 | 4 | 6 | 24 | 25.0% | Spy | 4 | 41 | 41 | 100.0% |
| Nme2 | 5 | 8 | 14 | 57.1% | Spy | 5 | 52 | 52 | 100.0% |
| Nme2 | 6 | 8 | 14 | 57.1% | Spy | 6 | 39 | 39 | 100.0% |
| Nme2 | 7 | 8 | 18 | 44.4% | Spy | 7 | 37 | 37 | 100.0% |
| Nme2 | 8 | 5 | 18 | 27.8% | Spy | 8 | 42 | 42 | 100.0% |
| Nme2 | 9 | 7 | 28 | 25.0% | Spy | 9 | 38 | 38 | 100.0% |
| Nme2 | 10 | 14 | 17 | 82.4% | Spy | 10 | 33 | 45 | 73.3% |
| Nme2 | 11 | 15 | 21 | 71.4% | Spy | 11 | 28 | 48 | 58.3% |
| Nme2 | 12 | 12 | 16 | 75.0% | Spy | 12 | 8 | 57 | 14.0% |
| Nme2 | 13 | 6 | 10 | 60.0% | Spy | 13 | 0 | 63 | 0.0% |
| Nme2 | 14 | 11 | 11 | 100.0% | Spy | 14 | 1 | 55 | 1.8% |
| Nme2 | 15 | 21 | 23 | 91.3% | Spy | 15 | 0 | 45 | 0.0% |
| Nme2 | 16 | 17 | 19 | 89.5% | Spy | 16 | 0 | 50 | 0.0% |
| Nme2 | 17 | 11 | 16 | 68.8% | Spy | 17 | 0 | 42 | 0.0% |
| Nme2 | 18 | 2 | 16 | 12.5% | Spy | 18 | 0 | 59 | 0.0% |
| Nme2 | 19 | 2 | 12 | 16.7% | Spy | 19 | 0 | 62 | 0.0% |
| Nme2 | 20 | 6 | 16 | 37.5% | Spy | 20 | 0 | 54 | 0.0% |
| Nme2 | 21 | 4 | 20 | 20.0% | Spy | 21 | n/a | 0 | n/a |
| Nme2 | 22 | 0 | 19 | 0.0% | Spy | 22 | n/a | 0 | n/a |
| Nme2 | 23 | 0 | 16 | 0.0% | Spy | 23 | 0 | 23 | 0.0% |
| Nme2 | 24 | 0 | 20 | 0.0% | Spy | 24 | 0 | 46 | 0.0% |
| Nme2 | 25 | n/a | 0 | n/a | Spy | 25 | 0 | 123 | 0.0% |
| Nme2 | 26 | n/a | 0 | n/a | Spy | 26 | 0 | 127 | 0.0% |
| Nme2 | 27 | 0 | 10 | 0.0% | Spy | 27 | 0 | 47 | 0.0% |
| Nme2 | 28 | 0 | 8 | 0.0% | Spy | 28 | 0 | 38 | 0.0% |
| Nme2 | 29 | 0 | 45 | 0.0% | Spy | 29 | 0 | 37 | 0.0% |
| Nme2 | 30 | 0 | 45 | 0.0% | Spy | 30 | 0 | 51 | 0.0% |
| Nme2 | 31 | 0 | 9 | 0.0% | Spy | 31 | 0 | 44 | 0.0% |
| Nme2 | 32 | 0 | 12 | 0.0% | Spy | 32 | 0 | 42 | 0.0% |
| Nme2 | 33 | 0 | 22 | 0.0% | Spy | 33 | 0 | 34 | 0.0% |
| Nme2 | 34 | 0 | 24 | 0.0% | Spy | 34 | 0 | 39 | 0.0% |
| Nme2 | 35 | 0 | 18 | 0.0% | Spy | 35 | 0 | 43 | 0.0% |
| Nme2 | 36 | 0 | 13 | 0.0% | Spy | 36 | 0 | 37 | 0.0% |
| Nme2 | 37 | 0 | 13 | 0.0% | Spy | 37 | 0 | 32 | 0.0% |
| Nme2 | 38 | 0 | 9 | 0.0% | Spy | 38 | 0 | 33 | 0.0% |
| Nme2 | 39 | 0 | 16 | 0.0% | Spy | 39 | 0 | 50 | 0.0% |
| Nme2 | 40 | 0 | 6 | 0.0% | Spy | 40 | 0 | 33 | 0.0% |

### Example 41—CIITA Guide RNA Screening in T Cells with BC22n

Different sgRNAs were screened for their potency in knocking out the CIITA gene in human T cells using C to T base editing. The percentage of T cells negative for MHC class II and/or CD74 protein expression was assayed following CIITA editing following electroporation with mRNA and different sgRNAs.

### Example 41.1 T Cell Preparation

Healthy human donor apheresis was obtained commercially (Hemacare), and cells were washed and resuspended in CliniMACS® PBS/EDTA buffer (Miltenyi Biotec Cat. 130-070-525) and processed in a MultiMACS™ Cell 24 Separator Plus device (Miltenyi Biotec). T cells were isolated via positive selection using a Straight from Leukopak® CD4/CD8 MicroBead kit, human (Miltenyi Biotec Cat. 130-122-352). T cells were aliquoted and cryopreserved for future use in Cryostor® CS10 (StemCell Technologies Cat. 07930).

Upon thaw, T cells were plated at a density of 1.0×10\hat{ }6 cells/mL in T cell growth media (TCGM) composed of CTS OpTimizer T Cell Expansion SFM and T Cell Expansion Supplement (ThermoFisher Cat. A1048501), 5% human AB serum (GeminiBio, Cat. 100-512) 1× Penicillin-Streptomycin, 1× Glutamax, 10 mM HEPES, 200 U/mL recombinant human interleukin-2 (Peprotech, Cat. 200-02), 5 ng/mL recombinant human interleukin 7 (Peprotech, Cat. 200-07), and 5 ng/mL recombinant human interleukin 15 (Peprotech, Cat. 200-15). T cells were rested in this media for 24 hours, at which time they were activated with T Cell TransAct™, human reagent (Miltenyi, Cat. 130-111-160) added at a 1:100 ratio by volume. T cells were activated for 48 hours prior to electroporation.

### Example 41.2 T Cell Editing with RNA Electroporation

Solutions containing mRNA encoding BC22n (SEQ ID NO: 1) and UGI (SEQ ID NO: 34) were prepared in P3 buffer. One hundred μM of CIITA-targeting sgRNAs were removed from their storage plates and denatured for 2 minutes at 95° C. and incubated at room temperature for 5 minutes. Forty-eight hours post activation, T cells were harvested, centrifuged, and resuspended at a concentration of 12.5×10\hat{ }6 T cells/mL in P3 electroporation buffer (Lonza). For electroporation, 1×10\hat{ }5 T cells were mixed with 20 ng/μL of BC22n mRNAs, 20 ng/μL of UGI mRNA, and 20 pmols of sgRNA as described in Table 1 in a final volume of 20 μL of P3 electroporation buffer. This mix was transferred in duplicate to a 96-well Nucleofector™ plate and electroporated using the manufacturer's pulse code. Electroporated T cells were immediately rested in 80 μL of CTS Optimizer T cell growth media without cytokines for 15 minutes before being transferred to new flat-bottom 96-well plates containing an additional 80 μL of CTS Optimizer T cell growth media supplemented with 2× cytokines. The resulting plates were incubated at 37° C. for 10 days. On day 4 post-electroporation, cells were split 1:2 in 2 U-bottom plates. One plate was collected for NGS sequencing, while the other plate was replenished with CTS Optimizer fresh media with 1× cytokines. This plate was used for flow cytometry on Day 7.

### Example 41.3 Flow Cytometry and NGS Sequencing

On day 7 post-editing, T cells were assayed by flow cytometry to determine the surface expression of CD74 and HLA-DR, DP, DQ. Briefly, T cells were incubated for 30 minutes at 4° C. with a mixture of antibodies diluted in cell staining buffer (BioLegend, Cat. No. 420201). Antibodies against CD3 (BioLegend, Cat. No. 317336), CD4 (BioLegend, Cat. No. 317434), CD8 (BioLegend, Cat. No. 301046), and Viakrome (Beckman Coulter, Cat. No. C36628) were diluted at 1:100, and antibodies against HLA II-DR (BioLegend, Cat. No. 327018), HLA II-DP (BD Biosciences Cat No. 750872), HLA II-DQ (BioLegend, Cat. No. 561504), and CD74 (BioLegend, Cat. No. 326808) were diluted at 1:50. Cells were subsequently washed, resuspended in 100 μL of cell staining buffer and processed on a Cytoflex flow cytometer (Beckman Coulter). Flow cytometry data was analyzed using the FlowJo software package. T cells were gated based on size, shape, viability, CD8, HLA II-DP, HLA II-DQ, HLA II-DR, and CD74 expression.

TABLE 104
Percentage of cells negative for surface protein following
genomic editing of CIITA with BC22n. (n = 2)

| Guide | % HLA | | % HLA | | % HLA | | | |
|---|---|---|---|---|---|---|---|---|
| | II-DP- | | II-DQ- | | II-DR- | | % CD74- | |
| ID | Mean | SD | Mean | SD | Mean | SD | Mean | SD |
| G000502 | 68.70 | 3.54 | 76.30 | 4.24 | 76.70 | 3.96 | 66.25 | 5.87 |
| G016788 | 62.95 | 1.91 | 73.40 | 3.11 | 73.50 | 4.38 | 61.35 | 4.60 |
| G016053 | 70.25 | 6.72 | 71.50 | 4.95 | 73.90 | 4.10 | 60.20 | 6.08 |
| G016103 | 63.05 | 6.29 | 73.95 | 1.20 | 75.30 | 0.71 | 63.25 | 1.63 |
| G016114 | 65.80 | 1.98 | 74.70 | 4.38 | 75.85 | 5.30 | 65.25 | 5.87 |
| G016117 | 63.70 | 0.57 | 74.60 | 3.25 | 76.00 | 3.11 | 63.45 | 5.30 |
| G016034 | 85.55 | 2.19 | 86.30 | 1.13 | 87.95 | 0.07 | 80.30 | 0.14 |
| G016035 | 85.35 | 3.75 | 83.55 | 1.06 | 84.05 | 0.35 | 75.25 | 1.20 |
| G016039 | 74.95 | 0.35 | 78.20 | 0.71 | 78.50 | 0.28 | 68.90 | 1.27 |
| G016040 | 61.90 | 0.85 | 76.30 | 2.26 | 77.80 | 1.56 | 64.10 | 2.55 |
| G016041 | 68.60 | 1.84 | 77.30 | 0.85 | 76.75 | 0.35 | 64.20 | 0.00 |
| G016043 | 79.95 | 1.48 | 82.55 | 0.35 | 82.50 | 0.71 | 73.80 | 0.00 |
| G016044 | 87.84 | 3.17 | 87.80 | 1.56 | 88.65 | 1.34 | 83.75 | 1.91 |
| G016045 | 82.25 | 2.90 | 88.70 | 0.99 | 88.35 | 0.78 | 83.40 | 0.99 |
| G016047 | 76.85 | 0.21 | 85.40 | 0.28 | 85.05 | 0.21 | 79.20 | 0.00 |
| G016050 | 59.05 | 1.91 | 86.80 | 0.71 | 83.40 | 0.57 | 79.00 | 0.99 |
| G016052 | 76.85 | 0.21 | 79.25 | 0.78 | 80.55 | 0.92 | 70.85 | 1.34 |
| G016054 | 70.30 | 1.70 | 79.85 | 1.20 | 79.30 | 0.42 | 70.35 | 0.35 |
| G016055 | 73.35 | 1.34 | 82.15 | 2.19 | 82.15 | 1.77 | 73.10 | 1.56 |
| G016056 | 75.80 | 1.41 | 86.05 | 1.20 | 86.35 | 1.34 | 79.05 | 1.91 |
| G016057 | 77.90 | 0.71 | 83.95 | 0.35 | 84.45 | 0.21 | 75.30 | 0.99 |
| G016058 | 83.65 | 2.19 | 87.25 | 1.06 | 88.20 | 0.99 | 81.20 | 1.41 |
| G016060 | 72.55 | 0.78 | 82.70 | 1.84 | 83.05 | 2.47 | 73.55 | 2.76 |
| G016061 | 73.15 | 6.29 | 83.10 | 0.57 | 82.55 | 0.21 | 74.50 | 0.42 |
| G016063 | 74.60 | 7.35 | 83.75 | 0.64 | 83.50 | 0.71 | 75.45 | 0.35 |
| G016064 | 97.98 | 0.17 | 97.80 | 0.18 | 96.83 | 0.13 | 98.58 | 0.04 |
| G016065 | 77.80 | 0.28 | 77.70 | 1.98 | 80.00 | 1.56 | 69.35 | 3.18 |
| G016068 | 97.73 | 0.26 | 98.07 | 0.81 | 97.59 | 0.66 | 98.55 | 0.66 |
| G016071 | 87.05 | 4.31 | 88.55 | 0.49 | 89.45 | 0.49 | 84.80 | 0.85 |
| G016074 | 96.88 | 0.19 | 96.34 | 0.04 | 95.85 | 0.57 | 96.56 | 0.23 |
| G016075 | 86.05 | 0.92 | 88.20 | 0.85 | 88.50 | 0.14 | 83.50 | 1.27 |
| G016076 | 96.69 | 0.64 | 96.67 | 0.01 | 96.38 | 0.02 | 96.34 | 0.18 |
| G016077 | 92.27 | 2.50 | 91.20 | 1.22 | 91.40 | 1.44 | 89.39 | 1.68 |
| G016078 | 71.20 | 0.28 | 79.55 | 1.48 | 80.65 | 1.48 | 70.40 | 0.85 |
| G016079 | 89.31 | 1.57 | 91.24 | 0.55 | 90.24 | 0.00 | 88.40 | 0.14 |
| G016081 | 74.35 | 0.07 | 83.05 | 1.06 | 83.00 | 0.42 | 73.20 | 1.41 |
| G016082 | 82.30 | 5.09 | 87.50 | 0.71 | 88.80 | 0.14 | 82.25 | 0.07 |
| G016083 | 74.95 | 4.88 | 82.90 | 0.57 | 83.95 | 0.21 | 74.65 | 1.06 |
| G016085 | 79.90 | 2.97 | 85.40 | 0.71 | 87.45 | 0.07 | 79.45 | 0.49 |
| G016086 | 97.71 | 0.33 | 98.06 | 0.44 | 96.63 | 0.08 | 98.89 | 0.18 |
| G016087 | 70.25 | 9.12 | 78.55 | 3.61 | 78.30 | 3.39 | 69.30 | 4.24 |
| G016088 | 82.25 | 5.16 | 87.40 | 0.57 | 88.30 | 0.42 | 81.75 | 1.06 |
| G016089 | 69.00 | 1.27 | 76.65 | 1.34 | 79.00 | 1.70 | 67.35 | 1.06 |
| G016091 | 95.35 | 0.95 | 96.81 | 0.24 | 96.64 | 0.25 | 97.15 | 0.28 |
| G016092 | 94.89 | 0.61 | 94.87 | 0.45 | 95.35 | 0.83 | 94.65 | 0.37 |
| G016093 | 91.33 | 0.31 | 92.35 | 0.26 | 93.94 | 0.23 | 90.51 | 0.37 |
| G016094 | 79.70 | 4.24 | 84.85 | 2.05 | 85.70 | 2.69 | 78.75 | 2.90 |
| G016095 | 83.00 | 2.12 | 90.55 | 0.23 | 90.54 | 0.24 | 85.25 | 0.49 |
| G016097 | 71.85 | 6.86 | 82.80 | 1.56 | 82.40 | 1.27 | 73.00 | 1.70 |
| G016098 | 74.40 | 5.66 | 82.35 | 3.32 | 83.50 | 2.69 | 74.55 | 4.45 |
| G016099 | 86.30 | 2.69 | 89.71 | 1.00 | 90.57 | 0.35 | 86.25 | 1.06 |
| G016100 | 76.65 | 1.34 | 83.90 | 2.69 | 86.25 | 2.19 | 77.25 | 3.04 |
| G016101 | 69.30 | 0.71 | 77.55 | 1.48 | 78.55 | 1.77 | 68.65 | 1.77 |
| G016102 | 71.00 | 1.84 | 80.70 | 0.99 | 80.60 | 1.56 | 70.10 | 0.99 |
| G016106 | 82.00 | 1.27 | 87.55 | 0.64 | 88.80 | 0.71 | 81.30 | 1.41 |
| G016108 | 88.24 | 3.73 | 91.49 | 0.02 | 91.42 | 0.66 | 87.00 | 0.85 |
| G016109 | 88.05 | 0.92 | 90.20 | 1.27 | 90.41 | 1.85 | 86.85 | 2.05 |
| G016110 | 88.50 | 3.25 | 91.12 | 0.99 | 90.14 | 1.05 | 87.35 | 1.20 |
| G016111 | 78.15 | 0.92 | 84.45 | 0.78 | 85.80 | 0.85 | 78.45 | 1.20 |
| G016112 | 72.20 | 3.82 | 79.85 | 2.33 | 81.70 | 2.26 | 73.05 | 2.47 |
| G016115 | 95.58 | 1.10 | 98.36 | 0.73 | 97.69 | 0.48 | 98.54 | 0.40 |
| G016116 | 88.95 | 0.35 | 91.15 | 2.47 | 92.29 | 1.79 | 88.08 | 2.94 |
| G016066 | 68.90 | 0.57 | 73.20 | 1.70 | 74.20 | 0.99 | 62.25 | 2.76 |
| G016113 | 93.60 | 1.15 | 93.02 | 0.98 | 93.75 | 0.66 | 92.13 | 1.20 |
| G016084 | 96.42 | 0.83 | 98.38 | 0.33 | 97.32 | 0.69 | 98.77 | 0.17 |
| G016104 | 84.95 | 1.77 | 89.69 | 1.53 | 91.20 | 0.97 | 86.00 | 1.41 |
| G016070 | 90.52 | 0.16 | 92.10 | 0.50 | 92.49 | 0.64 | 89.75 | 0.91 |
| G016090 | 96.41 | 1.27 | 98.06 | 0.08 | 97.43 | 0.15 | 98.66 | 0.10 |
| G016048 | 76.80 | 1.56 | 80.60 | 0.99 | 81.70 | 1.41 | 72.25 | 2.19 |
| G016051 | 65.85 | 20.44 | 83.15 | 1.63 | 84.45 | 1.34 | 75.20 | 2.40 |
| G016073 | 82.00 | 1.98 | 82.65 | 1.20 | 83.15 | 2.76 | 75.90 | 0.14 |
| G016037 | 80.55 | 1.91 | 78.05 | 0.35 | 80.15 | 0.07 | 70.20 | 0.57 |
| G016038 | 85.45 | 0.49 | 82.45 | 0.35 | 84.90 | 0.28 | 76.10 | 1.13 |
| G016046 | 90.10 | 0.05 | 90.75 | 0.47 | 91.09 | 0.86 | 87.45 | 0.78 |
| G016049 | 84.50 | 0.00 | 84.90 | 0.42 | 86.75 | 0.07 | 78.75 | 0.21 |
| G016036 | 85.05 | 1.06 | 83.15 | 1.48 | 85.00 | 0.99 | 76.45 | 2.47 |
| G016080 | 91.13 | 2.02 | 92.11 | 1.07 | 92.99 | 0.97 | 89.72 | 1.16 |
| G016096 | 75.00 | 4.38 | 82.90 | 3.82 | 83.45 | 3.61 | 75.65 | 4.31 |
| G016032 | 97.50 | 0.09 | 97.42 | 0.65 | 96.30 | 0.33 | 98.19 | 0.36 |
| G016033 | 91.32 | 0.88 | 87.75 | 0.49 | 88.20 | 0.28 | 84.10 | 0.14 |
| G016030 | 93.42 | 0.08 | 88.10 | 0.71 | 88.40 | 0.42 | 85.10 | 0.28 |
| G016067 | 96.69 | 0.25 | 95.82 | 0.69 | 95.14 | 0.64 | 96.30 | 0.48 |
| G016031 | 80.45 | 1.20 | 82.10 | 0.99 | 83.95 | 0.07 | 74.80 | 0.57 |
| G016062 | 74.80 | 11.17 | 86.60 | 0.28 | 86.75 | 0.35 | 80.15 | 0.35 |
| G016059 | 77.05 | 3.75 | 83.05 | 0.92 | 83.85 | 0.92 | 75.00 | 1.41 |
| G016105 | 66.55 | 5.87 | 74.90 | 3.68 | 76.50 | 3.68 | 65.25 | 3.75 |
| G016107 | 71.80 | 4.10 | 80.70 | 0.28 | 80.75 | 0.49 | 71.00 | 0.57 |
| G016042 | 95.06 | 0.02 | 95.90 | 0.30 | 94.17 | 0.35 | 94.89 | 1.02 |
| G016069 | 52.65 | 3.18 | 76.35 | 0.21 | 75.90 | 0.28 | 62.35 | 0.21 |
| G016072 | 46.45 | 4.45 | 73.50 | 0.57 | 71.35 | 0.92 | 56.55 | 0.64 |
| G018021 | 76.60 | 0.99 | 84.00 | 0.57 | 84.65 | 0.49 | 76.85 | 0.07 |
| G018022 | 60.00 | 3.39 | 72.20 | 1.13 | 73.25 | 1.34 | 60.35 | 2.62 |
| G018023 | 64.80 | 2.97 | 74.35 | 0.07 | 75.85 | 0.35 | 63.85 | 2.33 |
| G018024 | 64.10 | 1.70 | 74.80 | 1.84 | 74.90 | 1.98 | 63.75 | 4.03 |
| G018025 | 63.85 | 6.15 | 73.50 | 6.08 | 74.90 | 6.36 | 63.75 | 6.72 |
| G018026 | 72.15 | 4.17 | 81.35 | 0.92 | 82.55 | 1.48 | 73.00 | 0.71 |
| G018027 | 82.40 | 0.99 | 81.45 | 1.91 | 82.40 | 2.26 | 72.35 | 3.32 |
| G018028 | 78.25 | 0.49 | 78.40 | 0.00 | 80.05 | 0.49 | 67.90 | 2.40 |
| G018029 | 75.60 | 3.11 | 74.80 | 0.14 | 76.95 | 0.07 | 64.15 | 2.05 |
| G018030 | 76.70 | 1.56 | 76.35 | 3.46 | 77.80 | 3.54 | 66.65 | 5.44 |
| G018031 | 81.40 | 3.96 | 84.00 | 1.13 | 82.40 | 0.42 | 75.55 | 0.49 |
| G018032 | 77.00 | 2.97 | 79.70 | 0.14 | 79.30 | 0.42 | 70.35 | 0.92 |
| G018033 | 81.15 | 3.04 | 82.05 | 0.35 | 81.60 | 0.57 | 73.20 | 1.27 |
| G018034 | 96.87 | 0.43 | 93.49 | 0.88 | 92.67 | 0.99 | 92.96 | 1.63 |
| G018035 | 94.39 | 0.64 | 88.20 | 1.13 | 88.64 | 2.03 | 83.70 | 2.97 |
| G018036 | 73.80 | 0.28 | 76.45 | 2.19 | 77.55 | 2.90 | 65.30 | 3.82 |
| G018037 | 71.80 | 0.14 | 72.25 | 5.16 | 73.85 | 5.73 | 61.40 | 7.64 |
| G018038 | 70.25 | 7.85 | 74.95 | 1.06 | 75.65 | 2.05 | 63.65 | 2.62 |
| G018039 | 94.72 | 0.49 | 91.67 | 0.05 | 91.17 | 0.51 | 89.20 | 0.99 |
| G018040 | 77.30 | 1.13 | 81.70 | 0.42 | 81.60 | 0.57 | 72.35 | 0.64 |
| G018041 | 75.10 | 1.70 | 80.15 | 0.35 | 80.15 | 0.21 | 70.50 | 0.85 |
| G018042 | 89.35 | 0.49 | 86.60 | 0.14 | 87.90 | 0.14 | 80.50 | 0.00 |
| G018043 | 85.50 | 1.13 | 84.00 | 1.56 | 84.95 | 0.35 | 77.55 | 0.64 |
| G018044 | 88.20 | 0.99 | 89.01 | 1.57 | 89.25 | 0.78 | 83.95 | 2.19 |
| G018045 | 76.15 | 1.34 | 82.35 | 2.33 | 81.60 | 2.26 | 72.50 | 3.25 |
| G018046 | 87.20 | 0.42 | 88.65 | 0.21 | 89.00 | 0.57 | 82.80 | 1.27 |
| G018047 | 79.95 | 1.63 | 84.60 | 2.12 | 84.25 | 2.76 | 76.25 | 3.61 |
| G018048 | 88.50 | 2.12 | 88.00 | 1.13 | 89.50 | 0.42 | 81.60 | 0.14 |
| G018049 | 81.40 | 3.25 | 81.40 | 4.24 | 82.60 | 5.23 | 74.00 | 5.66 |
| G018050 | 77.75 | 0.49 | 78.60 | 0.85 | 80.85 | 0.07 | 70.25 | 0.35 |
| G018051 | 81.20 | 1.56 | 81.95 | 1.48 | 84.20 | 1.13 | 73.55 | 3.04 |
| G018052 | 76.30 | 1.56 | 81.70 | 1.27 | 81.10 | 1.56 | 70.90 | 2.69 |
| G018053 | 77.25 | 1.06 | 81.40 | 0.85 | 82.35 | 1.06 | 71.55 | 1.63 |
| G018054 | 90.23 | 0.14 | 89.00 | 0.99 | 89.25 | 0.64 | 84.50 | 0.28 |
| G018055 | 73.80 | 3.39 | 79.20 | 0.99 | 79.85 | 1.06 | 67.80 | 1.41 |
| G018056 | 73.40 | 2.97 | 83.15 | 0.35 | 83.15 | 0.78 | 73.15 | 0.78 |
| G018057 | 73.95 | 1.63 | 81.40 | 2.97 | 81.45 | 1.91 | 71.30 | 3.11 |
| G018058 | 73.40 | 3.25 | 80.90 | 0.99 | 81.05 | 0.07 | 69.15 | 0.35 |
| G018059 | 75.15 | 4.17 | 81.70 | 0.14 | 81.55 | 0.07 | 71.10 | 1.13 |
| G018060 | 75.90 | 2.69 | 81.30 | 0.28 | 81.55 | 0.78 | 72.05 | 1.91 |
| G018061 | 71.65 | 2.05 | 81.60 | 1.41 | 80.65 | 1.06 | 72.00 | 1.41 |
| G018062 | 70.75 | 2.33 | 75.05 | 0.64 | 76.30 | 1.56 | 64.65 | 2.33 |
| G018063 | 78.45 | 0.92 | 80.95 | 0.92 | 82.20 | 0.85 | 72.50 | 0.14 |
| G018064 | 76.15 | 0.78 | 82.50 | 0.00 | 82.90 | 0.28 | 73.90 | 0.28 |
| G018065 | 77.65 | 0.21 | 82.60 | 0.42 | 83.85 | 0.35 | 74.05 | 0.07 |
| G018066 | 72.60 | 0.00 | 82.30 | 0.28 | 83.05 | 0.64 | 73.60 | 0.14 |
| G018067 | 97.57 | 0.81 | 98.24 | 0.19 | 97.73 | 0.28 | 98.83 | 0.04 |
| G018068 | 89.05 | 0.64 | 90.79 | 0.52 | 90.81 | 1.02 | 87.20 | 1.27 |
| G018069 | 75.90 | 2.55 | 81.55 | 0.92 | 82.15 | 0.35 | 71.85 | 1.20 |
| G018070 | 75.30 | 1.27 | 82.05 | 1.20 | 82.15 | 0.64 | 71.20 | 0.42 |
| G018071 | 77.40 | 3.39 | 83.35 | 1.77 | 84.75 | 1.91 | 74.65 | 0.92 |
| G018072 | 72.05 | 1.63 | 81.45 | 0.78 | 81.95 | 1.91 | 71.95 | 2.62 |
| G018073 | 72.35 | 0.07 | 79.30 | 1.13 | 79.25 | 2.19 | 69.40 | 2.26 |
| G018074 | 67.25 | 2.05 | 75.25 | 0.49 | 76.40 | 0.57 | 64.35 | 0.35 |
| G018075 | 96.86 | 1.46 | 97.33 | 0.14 | 96.08 | 0.31 | 98.35 | 0.04 |
| G018076 | 94.26 | 4.87 | 97.96 | 0.21 | 97.57 | 0.15 | 98.55 | 0.06 |
| G018077 | 74.75 | 2.76 | 81.65 | 0.35 | 82.25 | 0.21 | 72.25 | 1.34 |
| G018078 | 94.30 | 0.48 | 94.87 | 0.31 | 95.03 | 0.06 | 93.43 | 0.24 |
| G018079 | 86.00 | 1.56 | 89.55 | 0.35 | 89.80 | 0.28 | 84.25 | 0.78 |
| G018080 | 80.50 | 3.96 | 85.50 | 1.70 | 85.45 | 1.63 | 78.35 | 0.78 |
| G018081 | 79.85 | 1.06 | 86.05 | 1.20 | 86.00 | 0.85 | 78.70 | 0.57 |
| G018082 | 80.10 | 0.14 | 83.10 | 0.42 | 84.05 | 1.20 | 75.65 | 1.91 |
| G018083 | 86.75 | 1.34 | 89.50 | 0.28 | 89.35 | 0.21 | 84.55 | 0.21 |
| G018084 | 84.60 | 3.25 | 87.35 | 1.34 | 87.95 | 0.92 | 82.00 | 1.27 |
| G018085 | 86.95 | 2.76 | 90.35 | 2.05 | 90.03 | 1.03 | 86.30 | 2.26 |
| G018086 | 68.15 | 1.34 | 78.60 | 0.42 | 75.95 | 0.07 | 66.25 | 0.92 |
| G018087 | 61.40 | 2.26 | 74.35 | 2.33 | 72.65 | 2.62 | 61.85 | 1.63 |
| G018088 | 88.80 | 0.85 | 91.11 | 0.28 | 91.88 | 0.11 | 87.60 | 1.41 |
| G018089 | 81.75 | 4.17 | 89.00 | 1.83 | 89.28 | 1.53 | 83.75 | 1.77 |
| G018090 | 81.65 | 1.20 | 90.11 | 0.29 | 89.98 | 0.25 | 85.35 | 1.34 |
| G018091 | 97.02 | 0.49 | 98.63 | 0.03 | 98.15 | 0.12 | 98.98 | 0.08 |
| G018092 | 71.40 | 5.23 | 81.90 | 0.71 | 82.40 | 0.85 | 73.45 | 0.92 |
| G018093 | 94.15 | 1.20 | 96.36 | 0.21 | 96.36 | 0.02 | 95.70 | 0.18 |
| G018094 | 77.50 | 2.12 | 84.55 | 1.63 | 85.25 | 1.48 | 76.50 | 0.14 |
| G018095 | 69.50 | 0.00 | 83.05 | 0.07 | 82.70 | 0.14 | 73.90 | 0.99 |
| G018096 | 69.25 | 0.21 | 83.50 | 0.99 | 83.45 | 1.06 | 75.25 | 2.05 |
| G018097 | 68.55 | 4.74 | 81.60 | 2.83 | 82.20 | 1.84 | 73.25 | 3.75 |
| G018098 | 66.65 | 0.21 | 78.85 | 0.21 | 79.75 | 0.21 | 68.75 | 1.20 |
| G018099 | 65.05 | 3.18 | 73.70 | 4.95 | 74.55 | 4.31 | 62.40 | 4.53 |
| G018100 | 95.51 | 0.40 | 95.99 | 0.19 | 95.62 | 0.92 | 96.21 | 0.18 |
| G018101 | 93.00 | 0.33 | 93.70 | 0.01 | 94.03 | 0.25 | 93.11 | 0.28 |
| G018102 | 96.55 | 0.33 | 96.31 | 0.64 | 96.34 | 0.08 | 96.58 | 0.08 |
| G018103 | 96.92 | 1.46 | 97.58 | 0.02 | 97.53 | 0.08 | 98.36 | 0.40 |
| G018104 | 72.00 | 0.99 | 82.45 | 2.19 | 82.70 | 1.84 | 73.40 | 2.12 |
| G018105 | 71.60 | 1.98 | 82.20 | 0.71 | 81.80 | 0.28 | 73.30 | 0.00 |
| G018106 | 92.49 | 1.33 | 98.62 | 0.06 | 97.03 | 0.43 | 98.96 | 0.04 |
| G018107 | 96.55 | 1.25 | 98.14 | 0.56 | 97.53 | 0.70 | 98.98 | 0.08 |
| G018108 | 85.50 | 0.42 | 88.00 | 0.71 | 89.35 | 0.92 | 83.40 | 1.56 |
| G018109 | 84.10 | 1.56 | 84.55 | 2.19 | 86.90 | 1.27 | 79.75 | 3.32 |
| G018110 | 67.65 | 3.04 | 75.15 | 0.49 | 77.50 | 0.99 | 65.35 | 1.48 |
| G018111 | 96.49 | 0.35 | 96.85 | 1.05 | 95.24 | 1.32 | 97.03 | 0.52 |
| G018112 | 68.60 | 5.09 | 76.65 | 4.88 | 77.30 | 4.53 | 66.60 | 4.38 |
| G018113 | 72.75 | 3.89 | 79.60 | 3.54 | 80.15 | 2.76 | 70.30 | 3.96 |
| G018114 | 70.80 | 2.26 | 80.05 | 2.47 | 81.25 | 1.77 | 71.65 | 2.62 |
| G018115 | 62.40 | 1.41 | 77.40 | 0.57 | 76.90 | 1.27 | 67.00 | 1.56 |
| G018116 | 96.05 | 0.89 | 98.01 | 0.39 | 96.99 | 0.42 | 98.32 | 0.21 |
| G018117 | 96.93 | 1.27 | 97.63 | 0.52 | 97.19 | 0.87 | 98.96 | 0.25 |
| G018118 | 97.25 | 0.51 | 97.71 | 0.25 | 96.94 | 0.44 | 98.91 | 0.26 |
| G018119 | 94.78 | 1.02 | 96.29 | 1.11 | 95.57 | 1.05 | 97.00 | 1.20 |
| G018120 | 96.04 | 1.87 | 97.61 | 0.06 | 97.42 | 0.56 | 98.94 | 0.07 |
| G018121 | 95.26 | 0.62 | 97.12 | 0.41 | 96.44 | 0.23 | 97.85 | 0.45 |

On day 4 post-editing, DNA samples were subjected to PCR and subsequent NGS analysis, as described in Example 1. Table 105 shows CIITA editing outcomes in T cells edited with BC22n.

TABLE 105
Mean percent editing at CIITA locus with BC22n. (n = 2)

| Guide | % C to T | | % C to A/G | | % Indels | |
|---|---|---|---|---|---|---|
| ID | Mean | SD | Mean | SD | Mean | SD |
| G000502 | 84.63 | 2.39 | 0.82 | 0.19 | 1.26 | 0.37 |
| G016788 | 95.63 | 0.06 | 0.99 | 0.39 | 0.72 | 0.15 |
| G016053 | 94.77 | 1.07 | 1.66 | 0.43 | 0.71 | 0.02 |
| G016103 | 95.60 | 0.97 | 0.63 | 0.23 | 0.75 | 0.29 |
| G016114 | 96.42 | 0.23 | 0.66 | 0.59 | 0.65 | 0.09 |
| G016117 | 96.23 | 0.70 | 0.82 | 0.28 | 0.90 | 0.17 |
| G016034 | 93.11 | 1.07 | 0.61 | 0.21 | 1.49 | 0.52 |
| G016035 | 95.41 | 0.59 | 0.52 | 0.31 | 0.62 | 0.24 |
| G016039 | 88.20 | 1.33 | 1.51 | 0.42 | 7.99 | 1.32 |
| G016043 | 88.51 | 2.91 | 1.27 | 0.11 | 2.23 | 0.46 |
| G016044 | 0.13 | 0.03 | 90.04 | 0.38 | 9.83 | 0.41 |
| G016045 | 61.66 | 0.51 | 1.73 | 0.90 | 7.12 | 0.22 |
| G016047 | 92.74 | 0.48 | 0.80 | 0.06 | 3.00 | 0.13 |
| G016050 | 90.63 | 0.04 | 1.35 | 0.38 | 1.87 | 0.56 |
| G016052 | 92.68 | 1.74 | 0.83 | 0.44 | 3.26 | 1.66 |
| G016054 | 90.82 | 2.94 | 1.03 | 0.89 | 4.38 | 4.28 |
| G016055 | 83.61 | 1.98 | 0.46 | 0.21 | 10.63 | 1.04 |
| G016056 | 96.69 | 0.65 | 0.30 | 0.03 | 0.64 | 0.35 |
| G016057 | 93.83 | 0.35 | 0.98 | 0.18 | 0.99 | 0.47 |
| G016058 | 95.49 | 0.06 | 0.35 | 0.14 | 0.41 | 0.31 |
| G016060 | 85.95 | 0.54 | 0.49 | 0.16 | 7.63 | 1.63 |
| G016061 | 92.81 | 2.51 | 0.59 | 0.34 | 0.75 | 0.19 |
| G016063 | 70.60 | 1.87 | 0.31 | 0.15 | 0.25 | 0.07 |
| G016064 | 94.14 | 2.28 | 0.81 | 0.41 | 0.97 | 0.33 |
| G016065 | 57.50 | 1.68 | 0.61 | 0.37 | 1.15 | 0.32 |
| G016068 | 94.26 | 0.97 | 0.34 | 0.16 | 0.52 | 0.21 |
| G016071 | 93.78 | 0.73 | 0.84 | 0.14 | 2.17 | 0.40 |
| G016074 | 93.88 | 1.20 | 0.92 | 0.19 | 2.41 | 0.28 |
| G016075 | 91.72 | 0.73 | 0.58 | 0.15 | 2.89 | 0.52 |
| G016076 | 91.24 | 1.10 | 0.28 | 0.20 | 3.02 | 1.02 |
| G016077 | 94.94 | 1.07 | 1.08 | 0.53 | 1.50 | 0.46 |
| G016078 | 93.52 | 1.78 | 0.41 | 0.15 | 3.30 | 1.54 |
| G016079 | 96.29 | 0.05 | 0.51 | 0.08 | 0.81 | 0.13 |
| G016081 | 75.32 | 7.28 | 1.33 | 1.44 | 7.61 | 7.04 |
| G016082 | 31.35 | 5.20 | 0.07 | 0.14 | 34.39 | 6.24 |
| G016083 | 0.24 | 0.08 | 99.63 | 0.11 | 0.08 | 0.06 |
| G016085 | 80.16 | 3.07 | 0.74 | 0.37 | 0.36 | 0.12 |
| G016086 | 96.02 | 1.68 | 1.45 | 0.46 | 0.84 | 0.38 |
| G016087 | 90.30 | 1.43 | 0.32 | 0.20 | 5.11 | 0.54 |
| G016088 | 92.14 | 0.51 | 1.05 | 0.33 | 2.24 | 0.86 |
| G016089 | 94.49 | 0.39 | 0.61 | 0.30 | 1.14 | 0.30 |
| G016091 | 95.93 | 0.99 | 1.03 | 0.23 | 0.37 | 0.09 |
| G016092 | 95.62 | 0.79 | 1.17 | 0.40 | 0.71 | 0.28 |
| G016093 | 95.74 | 0.81 | 0.94 | 0.43 | 0.43 | 0.18 |
| G016094 | 95.85 | 1.03 | 0.58 | 0.39 | 0.99 | 0.48 |
| G016095 | 94.77 | 0.52 | 1.32 | 0.31 | 0.72 | 0.37 |
| G016097 | 94.90 | 1.64 | 0.55 | 0.19 | 1.40 | 0.51 |
| G016098 | 91.71 | 1.11 | 0.48 | 0.18 | 0.50 | 0.36 |
| G016099 | 93.42 | 1.55 | 0.56 | 0.05 | 3.64 | 1.06 |
| G016100 | 96.54 | 0.93 | 0.46 | 0.22 | 0.46 | 0.36 |
| G016101 | 41.21 | 1.15 | 0.77 | 0.07 | 0.22 | 0.04 |
| G016102 | 96.06 | 1.23 | 0.46 | 0.05 | 0.69 | 0.25 |
| G016106 | 90.41 | 1.16 | 3.33 | 0.54 | 2.66 | 0.49 |
| G016108 | 76.19 | 0.74 | 0.42 | 0.28 | 0.69 | 0.38 |
| G016109 | 94.93 | 0.35 | 0.46 | 0.21 | 1.86 | 0.59 |
| G016110 | 87.64 | 1.01 | 0.55 | 0.20 | 7.63 | 0.72 |
| G016111 | 92.93 | 1.09 | 1.17 | 0.58 | 2.11 | 1.08 |
| G016112 | 1.56 | 0.46 | 1.79 | 0.90 | 0.03 | 0.05 |
| G016115 | 89.86 | 1.01 | 0.67 | 0.29 | 7.30 | 0.50 |
| G016116 | 91.37 | 0.33 | 0.54 | 0.27 | 0.59 | 0.09 |
| G016066 | 1.02 | 0.16 | 0.32 | 0.07 | 0.40 | 0.21 |
| G016113 | 93.23 | 0.98 | 1.10 | 0.16 | 2.40 | 0.57 |
| G016084 | 74.10 | 1.35 | 0.87 | 0.16 | 21.41 | 0.94 |
| G016104 | 0.00 | 0.00 | 0.00 | 0.00 | 0.00 | 0.00 |
| G016070 | 94.80 | 0.30 | 0.54 | 0.04 | 1.66 | 0.17 |
| G016090 | 84.09 | 0.00 | 0.51 | 0.00 | 12.29 | 0.00 |
| G016048 | 48.81 | 1.82 | 0.78 | 0.07 | 0.54 | 0.18 |
| G016051 | 95.69 | 0.45 | 1.06 | 0.09 | 0.91 | 0.25 |
| G016073 | 94.11 | 0.83 | 0.76 | 0.18 | 1.37 | 0.29 |
| G016037 | 64.35 | 3.31 | 1.61 | 0.81 | 3.46 | 4.94 |
| G016038 | 94.99 | 2.01 | 1.80 | 0.64 | 1.25 | 0.88 |
| G016046 | 82.29 | 2.89 | 0.88 | 0.54 | 14.28 | 2.86 |
| G016049 | 95.33 | 0.15 | 1.41 | 0.87 | 0.46 | 0.66 |
| G016036 | 71.71 | 3.82 | 0.63 | 0.14 | 1.94 | 0.40 |
| G016080 | 81.09 | 1.58 | 1.33 | 0.47 | 5.64 | 0.94 |
| G016096 | 94.79 | 0.33 | 0.45 | 0.10 | 1.81 | 0.37 |
| G016032 | 90.27 | 1.53 | 2.25 | 2.37 | 2.97 | 0.38 |
| G016033 | 83.90 | 0.89 | 1.79 | 1.63 | 5.03 | 0.94 |
| G016030 | 96.66 | 1.35 | 0.65 | 0.39 | 0.31 | 0.14 |
| G016067 | 95.79 | 0.26 | 0.61 | 0.17 | 1.37 | 0.26 |
| G016031 | 94.51 | 0.24 | 0.75 | 0.07 | 2.35 | 0.68 |
| G016062 | 93.37 | 0.45 | 1.17 | 0.13 | 1.91 | 0.33 |
| G016059 | 90.26 | 0.93 | 0.80 | 0.30 | 5.62 | 0.33 |
| G016105 | 95.81 | 1.17 | 0.53 | 0.24 | 0.47 | 0.45 |
| G016107 | 91.94 | 2.74 | 0.66 | 0.36 | 3.18 | 2.72 |
| G016042 | 90.07 | 0.00 | 0.97 | 0.00 | 2.51 | 0.00 |
| G016069 | 93.31 | 0.96 | 0.96 | 0.31 | 1.03 | 0.33 |
| G016072 | 84.94 | 7.39 | 0.39 | 0.08 | 11.91 | 8.00 |
| G018021 | 91.14 | 2.38 | 2.45 | 0.52 | 3.22 | 2.60 |
| G018022 | 93.68 | 3.27 | 1.83 | 0.33 | 1.46 | 0.10 |
| G018023 | 92.37 | 1.73 | 2.25 | 0.07 | 2.31 | 0.52 |
| G018024 | 96.06 | 1.54 | 0.41 | 0.37 | 0.97 | 0.07 |
| G018025 | 96.88 | 0.78 | 0.63 | 0.12 | 0.43 | 0.06 |
| G018026 | 89.15 | 1.37 | 1.66 | 0.04 | 7.20 | 1.31 |
| G018027 | 88.89 | 3.13 | 1.02 | 0.04 | 5.37 | 2.62 |
| G018028 | 91.28 | 2.09 | 0.59 | 0.24 | 2.96 | 2.02 |
| G018029 | 22.86 | 0.72 | 0.43 | 0.07 | 0.17 | 0.02 |
| G018030 | 79.89 | 2.66 | 0.36 | 0.51 | 0.18 | 0.25 |
| G018031 | 90.43 | 1.35 | 0.47 | 0.38 | 1.52 | 0.07 |
| G018032 | 64.78 | 2.64 | 1.39 | 0.69 | 1.06 | 0.50 |
| G018033 | 88.94 | 0.84 | 1.98 | 0.29 | 1.97 | 0.44 |
| G018034 | 89.00 | 1.56 | 1.09 | 0.11 | 6.23 | 0.88 |
| G018035 | 95.10 | 1.59 | 0.50 | 0.25 | 0.66 | 0.17 |
| G018036 | 83.69 | 0.01 | 1.91 | 0.30 | 0.53 | 0.20 |
| G018037 | 94.50 | 0.54 | 1.64 | 0.03 | 1.18 | 0.23 |
| G018038 | 90.91 | 0.00 | 1.11 | 0.00 | 3.74 | 0.00 |
| G018039 | 94.88 | 0.00 | 0.60 | 0.00 | 0.41 | 0.00 |
| G018040 | 44.77 | 1.52 | 0.33 | 0.14 | 0.50 | 0.03 |
| G018041 | 66.56 | 2.05 | 0.60 | 0.30 | 0.27 | 0.20 |
| G018042 | 90.79 | 1.10 | 1.28 | 0.47 | 1.64 | 0.35 |
| G018043 | 94.94 | 0.55 | 0.57 | 0.03 | 0.40 | 0.06 |
| G018044 | 91.71 | 1.61 | 0.89 | 0.40 | 1.25 | 0.20 |
| G018045 | 90.26 | 1.59 | 1.46 | 0.54 | 3.91 | 1.89 |
| G018046 | 94.59 | 1.33 | 0.76 | 0.05 | 2.03 | 0.64 |
| G018047 | 93.84 | 1.32 | 0.46 | 0.08 | 2.03 | 0.64 |
| G018048 | 95.20 | 2.58 | 0.54 | 0.52 | 0.62 | 0.30 |
| G018049 | 89.68 | 2.09 | 0.17 | 0.23 | 0.45 | 0.30 |
| G018050 | 95.74 | 0.65 | 0.31 | 0.22 | 0.84 | 0.39 |
| G018051 | 92.47 | 0.90 | 0.54 | 0.76 | 0.73 | 0.31 |
| G018052 | 93.14 | 0.85 | 1.39 | 0.91 | 0.85 | 0.78 |
| G018053 | 78.88 | 3.77 | 0.74 | 0.13 | 0.52 | 0.34 |
| G018054 | 92.69 | 1.11 | 0.79 | 0.10 | 2.55 | 0.46 |
| G018055 | 89.68 | 1.52 | 0.48 | 0.57 | 1.05 | 0.36 |
| G018056 | 94.00 | 1.10 | 0.45 | 0.14 | 1.85 | 0.99 |
| G018057 | 89.87 | 0.49 | 0.72 | 0.04 | 1.31 | 0.31 |
| G018058 | 91.04 | 0.77 | 0.59 | 0.12 | 1.46 | 0.28 |
| G018059 | 49.64 | 3.56 | 0.46 | 0.19 | 1.56 | 0.43 |
| G018060 | 97.65 | 0.65 | 1.22 | 0.17 | 1.13 | 0.47 |
| G018061 | 92.52 | 0.29 | 0.89 | 0.33 | 2.76 | 0.52 |
| G018062 | 88.19 | 0.72 | 0.84 | 0.18 | 6.65 | 0.41 |
| G018063 | 92.94 | 0.63 | 1.54 | 0.20 | 0.94 | 0.38 |
| G018064 | 94.14 | 1.12 | 1.38 | 0.31 | 0.97 | 0.09 |
| G018065 | 93.20 | 1.50 | 1.32 | 0.57 | 0.91 | 0.23 |
| G018066 | 91.62 | 0.45 | 1.69 | 0.06 | 0.45 | 0.17 |
| G018067 | 92.72 | 1.43 | 3.06 | 0.48 | 0.42 | 0.15 |
| G018068 | 93.33 | 0.49 | 0.62 | 0.08 | 0.27 | 0.06 |
| G018069 | 93.83 | 0.77 | 0.94 | 0.33 | 0.99 | 0.07 |
| G018070 | 94.61 | 0.57 | 0.59 | 0.08 | 1.33 | 0.35 |
| G018071 | 87.61 | 0.46 | 0.26 | 0.22 | 1.10 | 0.29 |
| G018072 | 92.86 | 0.84 | 1.76 | 0.04 | 0.34 | 0.06 |
| G018073 | 87.03 | 0.52 | 1.48 | 0.33 | 0.40 | 0.15 |
| G018074 | 80.06 | 11.41 | 0.42 | 0.13 | 0.46 | 0.24 |
| G018075 | 92.85 | 0.27 | 0.59 | 0.37 | 0.61 | 0.08 |
| G018076 | 95.88 | 1.59 | 0.56 | 0.38 | 0.61 | 0.30 |
| G018077 | 0.23 | 0.12 | 0.55 | 0.26 | 0.13 | 0.08 |
| G018078 | 94.97 | 1.42 | 0.48 | 0.16 | 0.44 | 0.22 |
| G018079 | 89.45 | 1.79 | 0.38 | 0.26 | 0.81 | 0.47 |
| G018080 | 80.86 | 3.14 | 0.22 | 0.15 | 0.58 | 0.35 |
| G018081 | 93.61 | 0.63 | 0.17 | 0.14 | 0.91 | 0.44 |
| G018082 | 93.15 | 1.21 | 0.16 | 0.07 | 1.80 | 0.50 |
| G018083 | 78.69 | 0.88 | 0.29 | 0.13 | 2.73 | 0.50 |
| G018084 | 81.88 | 0.70 | 0.43 | 0.19 | 1.39 | 0.60 |
| G018085 | 93.17 | 1.26 | 0.44 | 0.16 | 2.62 | 0.43 |
| G018086 | 95.03 | 1.07 | 0.61 | 0.20 | 1.39 | 0.44 |
| G018087 | 94.37 | 1.49 | 1.07 | 0.79 | 1.41 | 0.21 |
| G018088 | 91.62 | 1.31 | 1.01 | 0.43 | 1.91 | 0.99 |
| G018089 | 94.41 | 1.17 | 0.75 | 0.41 | 3.04 | 1.52 |
| G018090 | 93.66 | 0.82 | 0.54 | 0.28 | 3.12 | 0.96 |
| G018091 | 96.82 | 0.00 | 0.60 | 0.00 | 0.77 | 0.00 |
| G018092 | 90.55 | 1.58 | 0.74 | 0.27 | 2.02 | 0.34 |
| G018093 | 94.46 | 0.49 | 1.13 | 0.13 | 0.96 | 0.28 |
| G018094 | 82.55 | 5.17 | 0.55 | 0.11 | 12.35 | 6.02 |
| G018095 | 94.60 | 0.85 | 1.94 | 0.68 | 0.75 | 0.21 |
| G018096 | 54.68 | 0.62 | 1.99 | 0.16 | 0.38 | 0.12 |
| G018097 | 91.89 | 2.73 | 0.96 | 0.89 | 1.01 | 0.80 |
| G018098 | 70.42 | 0.00 | 0.35 | 0.00 | 0.00 | 0.00 |
| G018099 | 96.84 | 0.16 | 0.43 | 0.10 | 0.64 | 0.16 |
| G018100 | 93.24 | 1.08 | 0.77 | 0.20 | 3.07 | 0.06 |
| G018101 | 92.34 | 0.51 | 2.16 | 0.73 | 3.06 | 0.06 |
| G018102 | 91.87 | 0.99 | 0.42 | 0.34 | 0.61 | 0.16 |
| G018103 | 94.50 | 1.22 | 0.59 | 0.22 | 1.25 | 0.53 |
| G018104 | 94.14 | 0.76 | 0.31 | 0.14 | 1.09 | 0.65 |
| G018105 | 95.51 | 0.98 | 0.42 | 0.14 | 0.82 | 0.21 |
| G018106 | 94.77 | 0.90 | 1.12 | 0.18 | 2.15 | 0.28 |
| G018107 | 94.66 | 0.57 | 0.80 | 0.03 | 2.09 | 0.04 |
| G018108 | 94.77 | 1.14 | 1.23 | 0.70 | 1.55 | 0.96 |
| G018109 | 94.40 | 1.37 | 1.06 | 0.91 | 1.80 | 0.67 |
| G018110 | 92.19 | 1.30 | 1.86 | 0.34 | 4.04 | 0.77 |
| G018111 | 91.96 | 3.12 | 0.72 | 0.17 | 5.54 | 3.11 |
| G018112 | 91.54 | 1.34 | 0.80 | 0.15 | 5.69 | 0.44 |
| G018113 | 96.42 | 0.45 | 0.43 | 0.22 | 1.40 | 0.35 |
| G018114 | 95.95 | 0.89 | 0.79 | 0.32 | 0.27 | 0.20 |
| G018115 | 92.89 | 1.00 | 0.59 | 0.16 | 1.10 | 0.40 |
| G018116 | 91.94 | 0.57 | 0.99 | 0.54 | 0.61 | 0.24 |
| G018117 | 95.48 | 1.10 | 0.62 | 0.31 | 0.55 | 0.31 |
| G018118 | 96.20 | 0.38 | 0.43 | 0.31 | 0.46 | 0.17 |
| G018119 | 83.25 | 1.31 | 0.43 | 0.08 | 0.44 | 0.25 |
| G018120 | 97.00 | 0.72 | 0.42 | 0.26 | 0.38 | 0.20 |
| G018121 | 95.42 | 0.58 | 0.50 | 0.13 | 0.42 | 0.15 |

### Example 42—Screening CIITA sgRNAs in Dose-Response with BC22n in T Cells

Highly efficient CIITA sgRNAs identified in Example 41 were further assayed for base editing efficacy at multiple guide concentrations in T cells. The potency of each was assayed for genome editing efficacy by NGS or by disruption of surface protein expression of HLA-DR, DP, DQ by flow cytometry.

### Example 42.1 T Cell Preparation

Healthy human donor apheresis was obtained commercially (Hemacare), and cells were washed and resuspended in in CliniMACS® PBS/EDTA buffer (Miltenyi Biotec Cat. 130-070-525) and processed in a MultiMACS™ Cell 24 Separator Plus device (Miltenyi Biotec). T cells were isolated via positive selection using a Straight from Leukopak® CD4/CD8 MicroBead kit, human (Miltenyi Biotec Cat. 130-122-352). T cells were aliquoted and cryopreserved for future use in Cryostor® CS10 (StemCell Technologies Cat. 07930).

Upon thaw, T cells were plated at a density of 1.0×10\hat{ }6 cells/mL in T cell growth media (TCGM) composed of CTS OpTmizer T Cell Expansion SFM and T Cell Expansion Supplement (ThermoFisher Cat. A1048501), 5% human AB serum (GeminiBio, Cat. 100-512), 1× Penicillin-Streptomycin, 1× Glutamax, 10 mM HEPES, 200 U/mL recombinant human interleukin-2 (Peprotech, Cat. 200-02), 5 ng/mL recombinant human interleukin 7 (Peprotech, Cat. 200-07), and 5 ng/mL recombinant human interleukin 15 (Peprotech, Cat. 200-15). T cells were rested in this media for 24 hours, at which time they were activated with T Cell TransAct™ human reagent (Miltenyi, Cat. 130-111-160) added at a 1:100 ratio by volume. T cells were activated for 48 hours prior to electroporation.

### Example 42.2 T Cell Editing with RNA Electroporation

Solutions containing mRNAs encoding BC22n (SEQ ID NO: 1) and UGI (SEQ ID NO: 34) were prepared in P3 buffer. 100 μM CIITA targeting sgRNAs were removed from their storage plates and denatured for 2 minutes at 95° C. and incubated at room temperature for 5 minutes. Forty-eight hours post activation, T cells were harvested, centrifuged, and resuspended at a concentration of 12.5×10\hat{ }⁶ T cells/mL in P3 electroporation buffer (Lonza). Each sgRNA was serially diluted in ratio of 1:2 in P3 electroporation buffer starting from 60 pmols in a 96-well PCR plate in duplicate. Following dilution, 1×10\hat{ }⁵ T cells, 20 ng/μL of BC22n mRNAs, and 20 ng/μL of UGI mRNA were mixed with sgRNA plate to make the final volume of 20 μL of P3 electroporation buffer. This mix was transferred to 4 corresponding 96-well Nucleofector™ plates and electroporated using the manufacturer's pulse code. Electroporated T cells were immediately rested in 80 μL of CTS Optimizer T cell growth media without cytokines for 15 minutes before being transferred to new flat-bottom 96-well plates containing an additional 80 μL of CTS OpTmizer T cell growth media supplemented with 2× cytokines. The resulting plates were incubated at 37° C. for 7 days. On day 4 post-electroporation, cells were

split 1:2 in two U-bottom plates, and one plate was collected for NGS sequencing, while the other plate was replenished with CTS Optimizer fresh media with 1× cytokines. This plate was used for flow cytometry on Day 7.

### Example 42.3 Flow Cytometry and NGS Sequencing

On day 7 post-editing, T cells were assayed by flow cytometry to determine surface expression of HLA-DR, DP, DQ. Briefly, T cells were incubated for 30 minutes at 4° C. with a mixture of antibodies diluted in cell staining buffer (BioLegend, Cat. No. 420201). Antibodies against CD3 (BioLegend, Cat. No. 317336), CD4 (BioLegend, Cat. No. 317434), CD8 (BioLegend, Cat. No. 301046), and Viakrome (Beckman Coulter, Cat. No. C36628) were diluted at 1:100, and antibodies against HLA II-DR, DP, DQ (BioLegend, Cat. No. 361714) were diluted at 1:50. Cells were subsequently washed, resuspended in 100 μL of cell staining buffer and processed on a Cytoflex flow cytometer (Beckman Coulter). Flow cytometry data was analyzed using the FlowJo software package. T cells were gated based on size, shape, viability, CD8, and HLA-DR, DP, DQ.

Table 106 shows CIITA editing outcomes and the percentage of T cells negative for HLA-DR, DP, DQ in T cells following base editing with BC22n.

TABLE 106
Percent editing and percent of HLA II-DP, DQ, DR negative cells following
CIITA editing with BC22n base editor

| | sgRNA | C > T | | | % HLA II-DR, DP, DQ | | | % CD74 | | |
|---|---|---|---|---|---|---|---|---|---|---|
| | (pmols) | Ave | SD | N | Ave | SD | N | Ave | SD | N |
| G016064 | 60 | 96.20% | 0.69% | 2 | 99.65 | 0.07 | 2 | 96.2 | 0.14 | 2 |
| | 30 | 94.85% | 0.69% | 2 | 99.15 | 0.35 | 2 | 92.65 | 0.21 | 2 |
| | 15 | 90.26% | 0.03% | 2 | 96.70 | 0.28 | 2 | 90.15 | 2.19 | 2 |
| | 7.5 | 72.95% | 1.76% | 2 | 87.45 | 1.20 | 2 | 80.95 | 3.32 | 2 |
| | 3.75 | 49.55% | 2.09% | 2 | 73.05 | 0.64 | 2 | 66.35 | 4.31 | 2 |
| | 1.88 | 28.63% | 1.34% | 2 | 59.40 | 1.70 | 2 | 55.2 | 5.8 | 2 |
| | 0.94 | 14.98% | 0.01% | 2 | 54.60 | 2.40 | 2 | 46.9 | 1.84 | 2 |
| | 0 | 0.26% | 0.02% | 2 | 46.70 | 5.52 | 2 | 45.15 | 0.49 | 2 |
| G016068 | 60 | 97.40% | 0.87% | 2 | 99.70 | 0.00 | 2 | 94.25 | 1.2 | 2 |
| | 30 | 90.31% | 0.83% | 2 | 93.15 | 0.07 | 2 | 85.15 | 1.2 | 2 |
| | 15 | 75.08% | 0.17% | 2 | 82.45 | 0.21 | 2 | 74.65 | 1.48 | 2 |
| | 7.5 | 49.64% | 2.08% | 2 | 68.65 | 1.91 | 2 | 57 | 1.27 | 2 |
| | 3.75 | 28.95% | 1.51% | 2 | 57.85 | 1.63 | 2 | 43.9 | 1.27 | 2 |
| | 1.88 | 14.90% | 0.58% | 2 | 51.10 | 2.26 | 2 | 35.85 | 3.75 | 2 |
| | 0.94 | 7.11% | 0.22% | 2 | 49.50 | 4.67 | 2 | 38.2 | 4.1 | 2 |
| | 0 | 0.21% | 0.02% | 2 | 51.50 | 1.13 | 2 | 44.8 | 1.56 | 2 |
| G016074 | 60 | 96.62% | | 2 | 96.95 | 0.21 | 2 | 90.6 | 0.28 | 2 |
| | 30 | 94.89% | 0.58% | 2 | 93.50 | 1.27 | 2 | 85.45 | 1.06 | 2 |
| | 15 | 89.79% | 3.18% | 2 | 88.50 | 0.28 | 2 | 80.35 | 0.64 | 2 |
| | 7.5 | 72.00% | 1.61% | 2 | 77.75 | 0.35 | 2 | 68.35 | 1.77 | 2 |
| | 3.75 | 47.99% | 0.39% | 2 | 65.30 | 1.84 | 2 | 53.05 | 1.91 | 2 |
| | 1.88 | 27.92% | 0.44% | 2 | 57.60 | 0.14 | 2 | 45 | 0.42 | 2 |
| | 0.94 | 13.11% | 1.79% | 2 | 54.30 | 0.71 | 2 | 37.9 | 0 | 2 |
| | 0 | 0.41% | 0.13% | 2 | 49.15 | 2.05 | 2 | 44.05 | 1.91 | 2 |
| G016076 | 60 | 96.03% | 0.07% | 2 | 94.30 | 0.14 | 2 | 90 | 0.71 | 2 |
| | 30 | 93.29% | ND | 2 | 89.10 | 0.99 | 2 | 81.65 | 1.48 | 2 |
| | 15 | 80.74% | 0.41% | 2 | 83.55 | 0.35 | 2 | 74.1 | 0.85 | 2 |
| | 7.5 | 51.33% | 1.82% | 2 | 69.05 | 1.91 | 2 | 57.7 | 2.97 | 2 |
| | 3.75 | 28.40% | 2.41% | 2 | 58.90 | 2.26 | 2 | 43.15 | 0.64 | 2 |
| | 1.88 | 15.12% | 1.91% | 2 | 56.05 | 0.78 | 2 | 38.45 | 1.77 | 2 |
| | 0.94 | 7.23% | 0.26% | 2 | 52.80 | 0.71 | 2 | 38.2 | 0.28 | 2 |
| | 0 | 0.27% | 0.00% | 2 | 47.70 | 1.70 | 2 | 43.5 | 3.68 | 2 |
| G016086 | 60 | 97.81% | 0.16% | 2 | 99.25 | 0.35 | 2 | 94.35 | 0.92 | 2 |
| | 30 | ND | ND | 2 | 98.65 | 0.07 | 2 | 90.25 | 1.2 | 2 |
| | 15 | ND | ND | 2 | 98.20 | 0.28 | 2 | 88.8 | 1.98 | 2 |
| | 7.5 | 97.19% | 1.91% | 2 | 95.85 | 0.07 | 2 | 87.5 | 0.14 | 2 |
| | 3.75 | ND | ND | 2 | 87.10 | 0.28 | 2 | 74.95 | 2.47 | 2 |
| | 1.88 | ND | ND | 2 | 72.45 | 0.07 | 2 | 60.75 | 2.76 | 2 |
| | 0.94 | ND | ND | 2 | 59.10 | 0.85 | 2 | 48.05 | 3.89 | 2 |
| | 0 | ND | ND | 2 | 50.65 | 2.76 | 2 | 46.15 | 2.19 | 2 |
| G016091 | 60 | ND | ND | 2 | 98.05 | 0.07 | 2 | 89.85 | 0.21 | 2 |
| | 30 | ND | ND | 2 | 96.95 | 0.07 | 2 | 87.9 | 0.85 | 2 |
| | 15 | ND | ND | 2 | 91.40 | 1.84 | 2 | 83.65 | 2.76 | 2 |
| | 7.5 | ND | ND | 2 | 82.00 | 1.27 | 2 | 74 | 3.11 | 2 |
| | 3.75 | ND | ND | 2 | 69.70 | 0.99 | 2 | 62.75 | 2.33 | 2 |
| | 1.88 | ND | ND | 2 | 59.40 | 1.41 | 2 | 54.4 | 3.54 | 2 |
| | 0.94 | ND | ND | 2 | 54.25 | 2.05 | 2 | 46.3 | 5.09 | 2 |
| | 0 | ND | ND | 2 | 46.30 | 2.26 | 2 | 48.3 | 4.67 | 2 |
| G016115 | 60 | 93.33% | 0.70% | 2 | 96.80 | 0.14 | 2 | 92.55 | 0.07 | 2 |
| | 30 | 94.46% | 0.23% | 2 | 92.70 | 1.41 | 2 | 87.8 | 1.98 | 2 |
| | 15 | 93.51% | 0.70% | 2 | 89.85 | 0.35 | 2 | 82.75 | 3.75 | 2 |
| | 7.5 | 90.13% | 0.37% | 2 | 83.35 | 0.35 | 2 | 78.35 | 2.47 | 2 |
| | 3.75 | 75.18% | 1.51% | 2 | 71.40 | 1.27 | 2 | 65.5 | 2.4 | 2 |
| | 1.88 | 51.65% | 1.64% | 2 | 64.20 | 4.95 | 2 | 56.1 | 1.84 | 2 |
| | 0.94 | 30.19% | 2.14% | 2 | 55.25 | 3.04 | 2 | 49.15 | 4.88 | 2 |
| | 0 | 0.26% | 0.03% | 2 | 46.85 | 7.57 | 2 | 47.1 | 3.25 | 2 |
| G016084 | 60 | 73.43% | 0.13% | 2 | 97.00 | 0.14 | 2 | 91.35 | 1.06 | 2 |
| | 30 | 73.70% | 0.38% | 2 | 94.80 | 0.42 | 2 | 84.9 | 0.42 | 2 |
| | 15 | 67.30% | 1.19% | 2 | 91.60 | 0.71 | 2 | 80.6 | 0.57 | 2 |
| | 7.5 | 48.91% | 0.27% | 2 | 80.20 | 0.00 | 2 | 67.5 | 0.42 | 2 |
| | 3.75 | 28.24% | 0.35% | 2 | 67.50 | 0.57 | 2 | 51.85 | 3.18 | 2 |
| | 1.88 | 16.89% | 0.30% | 2 | 59.05 | 2.76 | 2 | 42.7 | 0.14 | 2 |
| | 0.94 | 7.42% | 0.06% | 2 | 54.65 | 0.92 | 2 | 40.05 | 0.78 | 2 |
| | 0 | 1.06% | 0.06% | 2 | 49.25 | 2.47 | 2 | 46.9 | 1.98 | 2 |
| G016090 | 60 | 87.05% | 2.52% | 2 | 98.70 | 1.27 | 2 | 93.25 | 2.19 | 2 |
| | 30 | 88.93% | 0.06% | 2 | 98.90 | 0.14 | 2 | 91 | 0.99 | 2 |
| | 15 | 89.51% | 0.24% | 2 | 98.70 | 0.28 | 2 | 90.45 | 0.21 | 2 |
| | 7.5 | 83.78% | 0.02% | 2 | 93.80 | 0.57 | 2 | 85.05 | 0.07 | 2 |
| | 3.75 | 66.23% | 0.40% | 2 | 83.35 | 1.06 | 2 | 72.35 | 0.35 | 2 |
| | 1.88 | 44.49% | 0.27% | 2 | 69.50 | 0.57 | 2 | 57.75 | 1.34 | 2 |
| | 0.94 | 22.90% | 1.49% | 2 | 57.25 | 1.77 | 2 | 45.15 | 2.47 | 2 |
| | 0 | 0.61% | 0.14% | 2 | 49.55 | 1.63 | 2 | 47.5 | 4.1 | 2 |
| G016032 | 60 | 91.66% | 0.76% | 2 | 99.35 | 0.07 | 2 | 93.65 | 2.05 | 2 |
| | 30 | 86.77% | 2.13% | 2 | 94.25 | 1.06 | 2 | 87.45 | 1.63 | 2 |
| | 15 | 75.23% | 1.22% | 2 | 86.10 | 2.26 | 2 | 74.65 | 0.78 | 2 |
| | 7.5 | 54.08% | 0.63% | 2 | 74.10 | 0.85 | 2 | 60.65 | 1.34 | 2 |
| | 3.75 | 33.34% | 2.39% | 2 | 63.20 | 3.82 | 2 | 45.95 | 3.75 | 2 |
| | 1.88 | 16.30% | 1.21% | 2 | 57.20 | 0.57 | 2 | 39.7 | 0.28 | 2 |
| | 0.94 | 7.33% | 0.38% | 2 | 53.10 | 0.85 | 2 | 37.6 | 1.13 | 2 |
| | 0 | 0.33% | 0.00% | 2 | 52.65 | 4.31 | 2 | 47.75 | 2.62 | 2 |
| G016067 | 60 | 97.37% | 0.56% | 2 | 93.10 | 0.14 | 2 | 85.95 | 2.19 | 2 |
| | 30 | 97.47% | 0.36% | 2 | 93.00 | 0.28 | 2 | 81.8 | 3.54 | 2 |
| | 15 | 95.78% | 0.13% | 2 | 89.30 | 1.41 | 2 | 79.45 | 0.49 | 2 |
| | 7.5 | 86.53% | 0.24% | 2 | 81.05 | 2.19 | 2 | 71 | 2.26 | 2 |
| | 3.75 | 68.12% | 1.27% | 2 | 69.35 | 1.20 | 2 | 58.3 | 2.4 | 2 |
| | 1.88 | 43.82% | 0.48% | 2 | 57.70 | 1.98 | 2 | 48.2 | 0.99 | 2 |
| | 0.94 | 22.80% | 1.26% | 2 | 54.95 | 0.35 | 2 | 42.55 | 1.34 | 2 |
| | 0 | 0.14% | 0.06% | 2 | 49.55 | 1.63 | 2 | 43.7 | 1.98 | 2 |
| G018067 | 60 | 95.58% | 0.35% | 2 | 99.40 | 0.00 | 2 | 91.45 | 1.34 | 2 |
| | 30 | 94.13% | 0.08% | 2 | 98.90 | 0.42 | 2 | 91.75 | 1.63 | 2 |
| | 15 | 91.49% | 0.05% | 2 | 95.95 | 0.21 | 2 | 89.45 | 0.07 | 2 |
| | 7.5 | 77.17% | 1.50% | 2 | 83.95 | 0.92 | 2 | 76.6 | 0.57 | 2 |
| | 3.75 | 56.15% | 1.07% | 2 | 71.00 | 0.71 | 2 | 63.65 | 4.03 | 2 |
| | 1.88 | 32.76% | 0.32% | 2 | 57.65 | 3.46 | 2 | 50.6 | 3.39 | 2 |
| | 0.94 | 17.47% | 0.95% | 2 | 50.85 | 3.04 | 2 | 46.1 | 3.82 | 2 |
| | 0 | 0.41% | 0.01% | 2 | 49.45 | 5.16 | 2 | 47.85 | 1.91 | 2 |
| G018075 | 60 | 96.12% | 1.35% | 2 | 98.25 | 0.07 | 2 | 92 | 1.27 | 2 |
| | 30 | 94.07% | 3.53% | 2 | 97.45 | 1.06 | 2 | 88.65 | 2.76 | 2 |
| | 15 | 92.08% | 0.90% | 2 | 97.10 | 0.14 | 2 | 87.25 | 2.05 | 2 |
| | 7.5 | 83.76% | 3.28% | 2 | 90.65 | 0.21 | 2 | 80.1 | 2.69 | 2 |
| | 3.75 | 67.35% | 2.13% | 2 | 78.85 | 0.21 | 2 | 67.1 | 2.97 | 2 |
| | 1.88 | 42.66% | 2.51% | 2 | 63.65 | 0.07 | 2 | 52.55 | 2.33 | 2 |
| | 0.94 | 25.54% | 0.32% | 2 | 56.55 | 3.75 | 2 | 43.1 | 3.25 | 2 |
| | 0 | 0.23% | 0.02% | 2 | 48.70 | 1.84 | 2 | 42.1 | 0.42 | 2 |
| G018091 | 60 | 97.80% | ND | 2 | 99.65 | 0.21 | 2 | 92.2 | 0.57 | 2 |
| | 30 | 96.32% | 0.91% | 2 | 97.50 | 0.28 | 2 | 88.7 | 1.7 | 2 |
| | 15 | 84.48% | 2.73% | 2 | 91.90 | 0.71 | 2 | 82.1 | 0.71 | 2 |
| | 7.5 | 76.56% | 0.49% | 2 | 78.05 | 1.63 | 2 | 64.15 | 0.35 | 2 |
| | 3.75 | 52.08% | 1.50% | 2 | 67.00 | 1.56 | 2 | 46.8 | 1.84 | 2 |
| | 1.88 | 30.00% | 2.36% | 2 | 58.80 | 2.55 | 2 | 40.7 | 2.4 | 2 |
| | 0.94 | 15.01% | 1.12% | 2 | 50.70 | 2.26 | 2 | 33 | 2.83 | 2 |
| | 0 | 0.00% | 0.00% | 2 | 47.90 | 1.70 | 2 | 39 | 0 | 2 |
| G018100 | 60 | 95.55% | 0.47% | 2 | 94.30 | 0.57 | 2 | 85.3 | 2.69 | 2 |
| | 30 | 95.22% | 1.02% | 2 | 91.75 | 0.78 | 2 | 79.1 | 0.85 | 2 |
| | 15 | 93.33% | 0.49% | 2 | 90.80 | 0.99 | 2 | 77.1 | 0.28 | 2 |
| | 7.5 | 88.77% | 0.15% | 2 | 84.40 | 0.85 | 2 | 69.9 | 1.56 | 2 |
| | 3.75 | 71.03% | 4.26% | 2 | 75.20 | 2.40 | 2 | 54.4 | 0.57 | 2 |
| | 1.88 | 46.87% | 1.95% | 2 | 63.80 | 0.28 | 2 | 45.7 | 0.28 | 2 |
| | 0.94 | 27.20% | 0.21% | 2 | 54.80 | 1.56 | 2 | 36.5 | 2.4 | 2 |
| | 0 | 0.17% | 0.04% | 2 | 52.25 | 1.63 | 2 | 42.8 | 0 | 2 |
| G018102 | 60 | 93.32% | 0.10% | 2 | 93.00 | 0.14 | 2 | 84.65 | 0.64 | 2 |
| | 30 | 77.08% | 2.22% | 2 | 80.40 | 1.84 | 2 | 68.7 | 2.55 | 2 |
| | 15 | 52.42% | 1.28% | 2 | 70.50 | 0.28 | 2 | 55.4 | 0.85 | 2 |
| | 7.5 | 31.87% | 0.20% | 2 | 58.80 | 4.53 | 2 | 39.4 | 4.38 | 2 |
| | 3.75 | 16.76% | 0.26% | 2 | 56.85 | 1.63 | 2 | 33.65 | 0.07 | 2 |
| | 1.88 | 8.18% | 0.06% | 2 | 54.15 | 0.21 | 2 | 30.35 | 0.92 | 2 |
| | 0.94 | 4.12% | 0.66% | 2 | 52.25 | 3.32 | 2 | 32.2 | 0.57 | 2 |
| | 0 | 0.28% | 0.12% | 2 | 50.00 | 3.68 | 2 | 42.45 | 4.17 | 2 |
| G018103 | 60 | 96.34% | 0.21% | 2 | 98.85 | 0.07 | 2 | 89.2 | 2.12 | 2 |
| | 30 | 90.93% | 0.17% | 2 | 95.15 | 0.92 | 2 | 84.95 | 0.07 | 2 |
| | 15 | 80.01% | 0.34% | 2 | 87.95 | 1.63 | 2 | 76.7 | 0.14 | 2 |
| | 7.5 | 59.92% | 0.48% | 2 | 70.90 | 2.97 | 2 | 56.45 | 3.18 | 2 |
| | 3.75 | 36.07% | 1.23% | 2 | 62.05 | 2.33 | 2 | 44.65 | 0.49 | 2 |
| | 1.88 | 19.59% | 2.15% | 2 | 58.65 | 2.76 | 2 | 38.2 | 1.41 | 2 |
| | 0.94 | 7.98% | 0.08% | 2 | 52.75 | 0.07 | 2 | 33.25 | 2.62 | 2 |
| | 0 | 0.22% | 0.03% | 2 | 50.95 | 0.64 | 2 | 43.1 | 2.69 | 2 |
| G018107 | 60 | ND | ND | 2 | 99.50 | 0.14 | 2 | 89.05 | 0.07 | 2 |
| | 30 | ND | ND | 2 | 99.05 | 0.07 | 2 | 91.1 | 0.85 | 2 |
| | 15 | ND | ND | 2 | 97.05 | 0.64 | 2 | 90 | 3.25 | 2 |
| | 7.5 | ND | ND | 2 | 90.45 | 0.07 | 2 | 83.25 | 2.76 | 2 |
| | 3.75 | ND | ND | 2 | 79.60 | 0.99 | 2 | 72.3 | 2.4 | 2 |
| | 1.88 | ND | ND | 2 | 66.20 | 2.26 | 2 | 58.7 | 4.81 | 2 |
| | 0.94 | ND | ND | 2 | 53.40 | 1.41 | 2 | 45.55 | 3.89 | 2 |
| | 0 | ND | ND | 2 | 51.65 | 2.05 | 2 | 47.35 | 4.03 | 2 |
| G018111 | 60 | 96.87% | 0.26% | 2 | 95.65 | 0.49 | 2 | 92.45 | 0.35 | 2 |
| | 30 | 95.44% | 0.07% | 2 | 89.40 | 0.71 | 2 | 86.5 | 0.42 | 2 |
| | 15 | 89.11% | 0.91% | 2 | 81.35 | 0.07 | 2 | 75.75 | 2.62 | 2 |
| | 7.5 | 71.93% | 0.55% | 2 | 70.05 | 3.32 | 2 | 61.15 | 0.21 | 2 |
| | 3.75 | 47.48% | 0.33% | 2 | 56.45 | 5.02 | 2 | 48.5 | 0.14 | 2 |
| | 1.88 | 25.72% | 1.46% | 2 | 59.00 | 3.11 | 2 | 44.95 | 0.07 | 2 |
| | 0.94 | 12.27% | 0.79% | 2 | 51.30 | 3.39 | 2 | 42.3 | 0.99 | 2 |
| | 0 | 0.15% | 0.01% | 2 | 51.00 | 3.25 | 2 | 45.1 | 1.41 | 2 |
| G018116 | 60 | 86.65% | 0.01% | 2 | 92.95 | 0.35 | 2 | 87.7 | 0.42 | 2 |
| | 30 | 62.59% | 0.13% | 2 | 79.90 | 1.84 | 2 | 69.75 | 2.9 | 2 |
| | 15 | 40.44% | 1.09% | 2 | 69.90 | 0.71 | 2 | 60.15 | 1.34 | 2 |
| | 7.5 | 23.19% | 2.46% | 2 | 60.35 | 4.74 | 2 | 45.45 | 0.92 | 2 |
| | 3.75 | 10.65% | 0.83% | 2 | 55.45 | 1.91 | 2 | 40.2 | 1.41 | 2 |
| | 1.88 | 5.63% | 0.47% | 2 | 56.85 | 1.06 | 2 | 40.75 | 0.64 | 2 |
| | 0.94 | 1.93% | 0.33% | 2 | 57.80 | 3.96 | 2 | 40 | 0.14 | 2 |
| | 0 | 0.19% | 0.10% | 2 | 50.55 | 0.21 | 2 | 47 | 1.41 | 2 |
| G018117 | 60 | 98.01% | 0.17% | 2 | 99.65 | 0.07 | 2 | 93.65 | 0.49 | 2 |
| | 30 | 97.14% | 0.69% | 2 | 99.20 | 0.14 | 2 | 92.35 | 0.07 | 2 |
| | 15 | 93.65% | 0.39% | 2 | 97.75 | 0.64 | 2 | 91.55 | 0.21 | 2 |
| | 7.5 | 81.77% | 0.50% | 2 | 91.60 | 0.42 | 2 | 81.85 | 0.35 | 2 |
| | 3.75 | 56.56% | 2.70% | 2 | 80.55 | 3.46 | 2 | 69.25 | 3.32 | 2 |
| | 1.88 | 33.70% | 3.00% | 2 | 69.30 | 1.13 | 2 | 56.8 | 2.69 | 2 |
| | 0.94 | 17.14% | 0.83% | 2 | 60.40 | 1.84 | 2 | 48.25 | 1.34 | 2 |
| | 0 | 0.18% | 0.01% | 2 | 51.95 | 2.76 | 2 | 46.5 | 1.13 | 2 |
| G018118 | 60 | 98.36% | 0.27% | 2 | 99.65 | 0.07 | 2 | 94.35 | 0.49 | 2 |
| | 30 | 97.70% | 0.19% | 2 | 99.45 | 0.21 | 2 | 88.35 | 8.13 | 2 |
| | 15 | 94.59% | 1.64% | 2 | 98.30 | 0.14 | 2 | 90.45 | 0.21 | 2 |
| | 7.5 | 83.77% | 2.47% | 2 | 92.80 | 1.13 | 2 | 81.2 | 0.57 | 2 |
| | 3.75 | 64.07% | 0.54% | 2 | 81.25 | 1.06 | 2 | 71.5 | 1.56 | 2 |
| | 1.88 | 40.72% | 2.16% | 2 | 70.65 | 2.90 | 2 | 61.15 | 3.18 | 2 |
| | 0.94 | 22.33% | 3.64% | 2 | 63.90 | 0.85 | 2 | 51.05 | 2.76 | 2 |
| | 0 | 0.14% | 0.02% | 2 | 52.95 | 1.48 | 2 | 47.85 | 1.77 | 2 |
| G018120 | 60 | 98.22% | 0.32% | 2 | 99.10 | 0.14 | 2 | 91.65 | 0.78 | 2 |
| | 30 | 96.96% | 0.43% | 2 | 99.15 | 0.07 | 2 | 89.1 | 0.57 | 2 |
| | 15 | 93.71% | 0.19% | 2 | 97.05 | 0.21 | 2 | 88.15 | 0.64 | 2 |
| | 7.5 | 82.19% | 1.50% | 2 | 90.25 | 0.78 | 2 | 80 | 0.99 | 2 |
| | 3.75 | 61.25% | 1.50% | 2 | 80.30 | 1.56 | 2 | 66.45 | 1.77 | 2 |
| | 1.88 | 37.98% | 1.29% | 2 | 68.85 | 0.78 | 2 | 52.75 | 3.04 | 2 |
| | 0.94 | 18.96% | 0.67% | 2 | 59.85 | 0.64 | 2 | 47.25 | 2.19 | 2 |
| | 0 | 0.17% | 0.02% | 2 | 49.60 | 0.57 | 2 | 46.1 | 0.99 | 2 |
| G018121 | 60 | 97.55% | 0.01% | 2 | 97.25 | 0.35 | 2 | 87.45 | 0.35 | 2 |
| | 30 | 92.24% | 0.98% | 2 | 92.50 | 0.71 | 2 | 83.6 | 3.96 | 2 |
| | 15 | 78.58% | 0.52% | 2 | 84.90 | 0.28 | 2 | 76.85 | 2.9 | 2 |
| | 7.5 | 57.37% | 0.50% | 2 | 73.75 | 3.04 | 2 | 64.5 | 3.25 | 2 |
| | 3.75 | 35.06% | 0.53% | 2 | 60.70 | 0.00 | 2 | 50.5 | 3.25 | 2 |
| | 1.88 | 18.80% | 2.81% | 2 | 61.00 | 1.41 | 2 | 45.95 | 4.31 | 2 |
| | 0.94 | 9.25% | 0.17% | 2 | 56.95 | 4.31 | 2 | 42.8 | 1.7 | 2 |
| | 0 | 0.21% | 0.00% | 2 | 47.45 | 3.04 | 2 | 44.55 | 1.34 | 2 |

### Example 43. Additional Embodiments

The following numbered embodiments provide additional support for and descriptions of the embodiments herein.
Embodiment A1. An mRNA comprising an open reading frame encoding a polypeptide comprising an APOBEC3A deaminase (A3A) and an RNA-guided nickase, wherein the polypeptide does not comprise a uracil glycosylase inhibitor (UGI).
Embodiment A2. A composition comprising a first mRNA comprising a first open reading frame encoding a polypeptide comprising an APOBEC3A deaminase (A3A) and an RNA-guided nickase, and a second mRNA comprising a second open reading frame encoding a uracil glycosylase inhibitor (UGI), wherein the second mRNA is different from the first mRNA, optionally wherein the composition comprises lipid nanoparticles.
Embodiment A3. The composition of embodiment A2, wherein the first polypeptide does not comprise a UGI.
Embodiment A4. The composition of embodiment A2 or A3, wherein the molar ratio of the second mRNA to the first mRNA is from 1:1 to 30:1.
Embodiment A5. A method of modifying a target gene comprising delivering to a cell a first mRNA comprising a first open reading frame encoding a first polypeptide comprising an APOBEC3A deaminase (A3A) and an RNA-guided nickase, a second mRNA comprising a second open reading frame encoding a uracil glycosylase inhibitor (UGI), wherein the second mRNA is different from the first mRNA, and at least one guide RNA (gRNA).
Embodiment A6. The composition or method of any one of embodiments A2-A5, wherein the molar ratio of the second mRNA to the first mRNA is from 1:1 to 30:1.
Embodiment A7. The composition or method of any one of embodiments A4-A6, wherein the molar ratio is from 2:1 to 30:1.
Embodiment A8. The composition or method of any one of embodiments A4-A6, wherein the molar ratio is from 7:1 to 22:1.
Embodiment A9. A cell comprising a composition comprising a first mRNA comprising a first open reading frame encoding a polypeptide comprising an APOBEC3A deaminase (A3A) and an RNA-guided nickase, and a second mRNA comprising a second open reading frame encoding a uracil glycosylase inhibitor (UGI), wherein the second mRNA is different from the first mRNA.
Embodiment A10. An engineered cell comprising at least one base edit and/or indel, wherein the base edit and/or indel is made by contacting a cell with a composition comprising a first mRNA comprising a first open reading frame encoding a polypeptide comprising an APOBEC3A deaminase (A3A) and an RNA-guided nickase, and a second mRNA comprising a second open reading frame encoding a uracil glycosylase inhibitor (UGI), wherein the second mRNA is different from the first mRNA.
Embodiment A11. The mRNA, composition, method, cell, or engineered cell of any one of the preceding embodiments, wherein the A3A comprises an amino acid sequence with at least 87% identity to SEQ ID NO: 40.
Embodiment A12. The mRNA, composition, method, cell, or engineered cell of any one of the preceding embodiments, wherein the A3A comprises an amino acid sequence with at least 90% identity to SEQ ID NO: 40.
Embodiment A13. The mRNA, composition, method, cell, or engineered cell of any one of the preceding embodiments, wherein the A3A comprises an amino acid sequence with at least 95% identity to SEQ ID NO: 40.
Embodiment A14. The mRNA, composition, method, cell, or engineered cell of any one of the preceding embodiments, wherein the A3A comprises an amino acid sequence with at least 98% identity to SEQ ID NO: 40.
Embodiment A15. The mRNA, composition, method, cell, or engineered cell of any one of the preceding embodiments, wherein the A3A comprises an amino acid sequence with at least 99% identity to SEQ ID NO: 40.
Embodiment A16. The mRNA, composition, method, cell, or engineered cell of any one of the preceding embodiments, wherein the A3A comprises the amino acid sequence of SEQ ID NO: 40.
Embodiment A17. The mRNA, composition, method, cell, or engineered cell of any one of the preceding embodiments, wherein the A3A is a human A3A.
Embodiment A18. The mRNA, composition, method, cell, or engineered cell of any one of the preceding embodiments, wherein the A3A is a wild-type A3A.
Embodiment A19. The composition, method, cell, or engineered cell of any one of the preceding embodiments, wherein the UGI comprises an amino acid sequence with at least 80% identity to SEQ ID NO: 27.
Embodiment A20. The composition, method, cell, or engineered cell of any one of the preceding embodiments, wherein the UGI comprises an amino acid sequence with at least 90% identity to SEQ ID NO: 27.
Embodiment A21. The composition, method, cell, or engineered cell of any one of the preceding embodiments, wherein the UGI comprises an amino acid sequence with at least 95% identity to SEQ ID NO: 27.
Embodiment A22. The composition, method, cell, or engineered cell of any one of the preceding embodiments, wherein the UGI comprises an amino acid sequence with at least 98% identity to SEQ ID NO: 27.
Embodiment A23. The composition, method, cell, or engineered cell of any one of the preceding embodiments, wherein the UGI comprises an amino acid sequence with at least 99% identity to SEQ ID NO: 27.
Embodiment A24. The composition, method, cell, or engineered cell of any one of the preceding embodiments, wherein the UGI comprises the amino acid sequence of SEQ ID NO: 27.
Embodiment A25. The composition, method, or cell of any one of the preceding embodiments, further comprising at least one guide RNA (gRNA).
Embodiment A26. The composition, method, cell, or engineered cell of any one of the preceding embodiments, comprising a gRNA, wherein the gRNA is an sgRNA.
Embodiment A27. The composition, method, cell, or engineered cell of any one of the preceding embodiments, comprising a gRNA, wherein the gRNA is a dgRNA.
Embodiment A28. The composition, method, cell, or engineered cell of any one of the preceding embodiments, comprising a gRNA, wherein the gRNA is a short-single guide RNA (short-sgRNA) comprising a conserved portion of an sgRNA comprising a hairpin region, wherein the hairpin region lacks at least 5-10 nucleotides and wherein the short-sgRNA comprises a 5′ end modification and/or a 3′ end modification.
Embodiment A29. The mRNA, composition, method, cell, or engineered cell of any one of the preceding embodiments, wherein the RNA-guided nickase is a Cas nickase.
Embodiment A30. The mRNA, composition, method, cell, or engineered cell of any one of the preceding embodiments, wherein the RNA-guided nickase is a Class 2 Cas nickase.
Embodiment A31. The mRNA, composition, method, cell, or engineered cell of any one of the preceding embodiments, wherein the RNA-guided nickase is a Cas9 nickase.
Embodiment A32. The mRNA, composition, method, cell, or engineered cell of any one of the preceding embodiments, wherein the RNA-guided nickase is an S. pyogenes Cas9 nickase.
Embodiment A33. The mRNA, composition, method, cell, or engineered cell of any one of the preceding embodiments, wherein the RNA-guided nickase is a D10A SpyCas9 nickase.
Embodiment A34. The mRNA, composition, method, cell, or engineered cell of any one of the preceding embodiments, wherein the RNA-guided nickase is a N. meningitidis Cas9 nickase.
Embodiment A35. The mRNA, composition, method, cell, or engineered cell of any one of the preceding embodiments, wherein the RNA-guided nickase is a D16A Nme2 Cas9 nickase.
Embodiment A36. The mRNA, composition, method, cell, or engineered cell of any one of the preceding embodiments, wherein the RNA-guided nickase comprises an amino acid sequence having at least 80%, 90%, 95%, 98%, or 99% identity to any one of SEQ ID NOs: 70, 73, or 76.
Embodiment A37. The mRNA, composition, method, cell, or engineered cell of any one of the preceding embodiments, wherein the RNA-guided nickase comprises the amino acid sequence of any one of SEQ ID NOs: 70, 73, or 76.
Embodiment A38. The mRNA, composition, method, cell, or engineered cell of any one of the preceding embodiments, wherein the sequence encoding the RNA-guided nickase comprises a nucleotide sequence having at least 80%, 90%, 95%, 98%, or 99% identity to the nucleotide sequence of any one of SEQ ID NOs: 72, 75, or 78.
Embodiment A39. The mRNA, composition, method, cell, or engineered cell of any one of the preceding embodiments, wherein the sequence encoding the RNA-guided nickase comprises a nucleotide sequence having at least 90% to the nucleotide sequence of any one of SEQ ID NOs: 72, 75, or 78.
Embodiment A40. The mRNA, composition, method, cell, or engineered cell of any one of the preceding embodiments, wherein the sequence encoding the RNA-guided nickase comprises a nucleotide sequence having at least 95% to the nucleotide sequence of any one of SEQ ID NOs: 72, 75, or 78.
Embodiment A41. The mRNA, composition, method, cell, or engineered cell of any one of the preceding embodiments, wherein the sequence encoding the RNA-guided nickase comprises a nucleotide sequence having at least 98% to the nucleotide sequence of any one of SEQ ID NOs: 72, 75, or 78.
Embodiment A42. The mRNA, composition, method, cell, or engineered cell of any one of the preceding embodiments, wherein the sequence encoding the RNA-guided nickase comprises a nucleotide sequence having at least 99% to the nucleotide sequence of any one of SEQ ID NOs: 72, 75, or 78.
Embodiment A43. The mRNA, composition, method, cell, or engineered cell of any one of the preceding embodiments, wherein the sequence encoding the RNA-guided nickase comprises the nucleotide sequence of any one of SEQ ID NOs: 71, 72, 74, 75, or 77-90.
Embodiment A44. The mRNA, composition, method, cell, or engineered cell of any one of the preceding embodiments, wherein the mRNA comprises a 5′ UTR with at least 90% identity to any one of SEQ ID NOs: 91-98.
Embodiment A45. The mRNA, composition, method, cell, or engineered cell of any one of the preceding embodiments, wherein the mRNA comprises a 3′ UTR with at least 90% identity to any one of SEQ ID NOs: 99-106.
Embodiment A46. The mRNA, composition, method, cell, or engineered cell of any one of the preceding embodiments, wherein the mRNA comprises a 5′ UTR and a 3′ UTR from the same source.
Embodiment A47. The mRNA, composition, method, cell, or engineered cell of any one of the preceding embodiments, wherein the mRNA comprises a 5′ cap selected from Cap0, Cap1, and Cap2.
Embodiment A48. The mRNA, composition, method, cell, or engineered cell of any one of the preceding embodiments, wherein the open reading frame encoding a polypeptide comprising an APOBEC3A deaminase (A3A) and an RNA-guided nickase, and/or the open reading frame encoding a uracil glycosylase inhibitor (UGI) comprise minimal adenine codons and/or minimal uridine codons.
Embodiment A49. The mRNA, composition, method, cell, or engineered cell of any one of the preceding embodiments, wherein the open reading frame encoding a polypeptide comprising an APOBEC3A deaminase (A3A) and an RNA-guided nickase, and/or the open reading frame encoding a uracil glycosylase inhibitor (UGI) comprise minimal adenine codons.
Embodiment A50. The mRNA, composition, method, cell, or engineered cell of any one of the preceding embodiments, wherein the open reading frame encoding a polypeptide comprising an APOBEC3A deaminase (A3A) and an RNA-guided nickase, and/or the open reading frame encoding a uracil glycosylase inhibitor (UGI) have codons that increase translation of the mRNA in a mammal.
Embodiment A51. The mRNA, composition, method, cell, or engineered cell of any one of the preceding embodiments, wherein the open reading frame encoding a polypeptide comprising an APOBEC3A deaminase (A3A) and an RNA-guided nickase, and/or the open reading frame encoding a uracil glycosylase inhibitor (UGI) have codons that increase translation of the mRNA in a mammal, wherein the mammal is a human.
Embodiment A52. The mRNA, composition, method, cell, or engineered cell of any one of the preceding embodiments, wherein the A3A is located N-terminal to the RNA-guided nickase in the polypeptide.
Embodiment A53. The mRNA, composition, method, cell, or engineered cell of any one of the preceding embodiments, wherein the encoded RNA-guided nickase comprises a nuclear localization signal (NLS).
Embodiment A54. The mRNA, composition, method, cell, or engineered cell of any one of the preceding embodiments, wherein the encoded RNA-guided nickase comprises a nuclear localization signal (NLS), and wherein the NLS is at the C-terminus of the RNA-guided nickase.
Embodiment A55. The mRNA, composition, method, cell, or engineered cell of any one of the preceding embodiments, wherein the encoded RNA-guided nickase comprises a nuclear localization signal (NLS), and wherein the NLS is at the N-terminus of the RNA-guided nickase, or wherein an NLS is fused to both the N-terminus and C-terminus of the RNA-guided nickase.
Embodiment A56. The mRNA, composition, method, cell, or engineered cell of any one of the preceding embodiments, wherein the encoded RNA-guided nickase comprises a nuclear localization signal (NLS), and wherein a linker is present between the N-terminus of the RNA-guided nickase and the NLS, optionally wherein the linker is a peptide linker.
Embodiment A57. The mRNA, composition, method, cell, or engineered cell of any one of the preceding embodiments, wherein the encoded RNA-guided nickase comprises a nuclear localization signal (NLS), and wherein the NLS comprises a sequence having at least 80%, 85%, 90%, or 95% identity to any one of SEQ ID NOs: 63 and 110-122.
Embodiment A58. The mRNA, composition, method, cell, or engineered cell of any one of the preceding embodiments, wherein the encoded RNA-guided nickase comprises a nuclear localization signal (NLS), and wherein the NLS comprises the sequence of any one of SEQ ID NOs: 63 and 110-122.
Embodiment A59. The mRNA, composition, method, cell, or engineered cell of any one of the preceding embodiments, wherein the encoded RNA-guided nickase comprises a nuclear localization signal (NLS), and wherein the NLS is encoded by a sequence having at least 80%, 85%, 90%, 95%, 98% or 100% identity to the sequence of any one of SEQ ID NOs: 123-135.
Embodiment A60. The mRNA, composition, method, cell, or engineered cell of any one of the preceding embodiments, wherein the encoded RNA-guided nickase comprises a nuclear localization signal (NLS), and wherein the A3A is located N-terminal to the NLS in the polypeptide.
Embodiment A61. The mRNA, composition, method, cell, or engineered cell of any one of the preceding embodiments, wherein the encoded RNA-guided nickase comprises a nuclear localization signal (NLS), and wherein the RNA-guided nickase is located N-terminal to the NLS in the polypeptide.
Embodiment A62. The mRNA, composition, method, cell, or engineered cell of any one of the preceding embodiments, wherein the open reading frame encoding a polypeptide comprising an APOBEC3A deaminase (A3A) and an RNA-guided nickase comprises a sequence having at least 80%, 85%, 90%, 95%, 98% or 100% identity to the sequence of SEQ ID NO:1.
Embodiment A63. The mRNA, composition, method, cell, or engineered cell of any one of the preceding embodiments, wherein the open reading frame encoding a polypeptide comprising an APOBEC3A deaminase (A3A) and an RNA-guided nickase comprises a sequence having at least 80%, 85%, 90%, 95%, 98% or 100% identity to the sequence of SEQ ID NO: 4.
Embodiment A64. The mRNA, composition, method, cell, or engineered cell of any one of the preceding embodiments, wherein at least 10% of the uridine in the mRNA is substituted with a modified uridine.
Embodiment A65. The mRNA, composition, method, cell or engineered cell of embodiment A64, wherein the modified uridine is one or more of N1-methyl-pseudouridine, pseudouridine, 5-methoxyuridine, or 5-iodouridine.
Embodiment A66. The mRNA, composition, method, cell, or engineered cell of embodiment A64 or A65, wherein the modified uridine is one or both of N1-methyl-pseudouridine or 5-methoxyuridine.
Embodiment A67. The mRNA, composition, method, cell, or engineered cell of any one of embodiments A64-A66, wherein the modified uridine is N1-methyl-pseudouridine.
Embodiment A68. The mRNA, composition, method, cell, or engineered cell of any one of embodiments A64-A66, wherein the modified uridine is 5-methoxyuridine.
Embodiment A69. The mRNA, composition, method, cell, or engineered cell of any one of embodiments A64-A68, wherein 15% to 45% of the uridine is substituted with the modified uridine.
Embodiment A70. The mRNA, composition, method, cell, or engineered cell of any one of embodiments A64-A69, wherein at least 20% or at least 30% of the uridine is substituted with the modified uridine.
Embodiment A71. The mRNA, composition, method, cell, or engineered cell of any one of embodiments A64-A70, wherein at least 80% or at least 90% of the uridine is substituted with the modified uridine.
Embodiment A72. The mRNA, composition, method, cell, or engineered cell of any one of embodiments A64-A71, wherein 100% uridine is substituted with the modified uridine.
Embodiment A73. The mRNA, composition, method, or cell of any one of the preceding embodiments, further encoding a peptide linker between the A3A and RNA-guided nickase, optionally wherein the peptide linker is XTEN.
Embodiment A74. The mRNA, composition, method, or cell of any one of the preceding embodiments, further encoding a peptide linker between the A3A and RNA-guided nickase, wherein the peptide linker comprises at least 1, at least 2, at least 3, at least 4, at least 5, at least 6, at least 7, at least 8, at least 9, at least 10, at least 15, at least 20, at least 25, at least 30, at least 40, at least 50, or more amino acids.
Embodiment A75. The mRNA, composition, method, or cell of any one of the preceding embodiments, further encoding a peptide linker between the A3A and RNA-guided nickase, wherein the peptide linker comprises one or more sequences selected from SEQ ID NOs: 46-59 and 211-267.
Embodiment A76. A polypeptide encoded by the mRNA of any one of the preceding embodiments A.
Embodiment A77. A vector comprising the mRNA of any one of the preceding embodiments A.
Embodiment A78. An expression construct comprising a promoter operably linked to a sequence encoding the mRNA of any one of the preceding embodiments A.
Embodiment A79. A plasmid comprising the expression construct of embodiment A78.
Embodiment A80. A host cell comprising the vector of embodiment A77, the expression construct of embodiment A78, or the plasmid of embodiment A79.
Embodiment A81. The mRNA or composition of any one of the preceding embodiments, wherein the mRNA or composition is formulated as a lipid nucleic acid assembly composition, optionally a lipid nanoparticle.
Embodiment A82. Use of the mRNA or composition according to any one of the preceding embodiments for modifying a target gene in a cell.
Embodiment A83. Use of the mRNA or composition according to any one of the preceding embodiments for the manufacture of a medicament for modifying a target gene in a cell.
Embodiment A84. A method of modifying a target gene in a cell, comprising delivering to the cell one or more lipid nucleic acid assembly compositions, optionally lipid nanoparticles, comprising:
   (a) a first mRNA comprising a first open reading frame encoding a polypeptide comprising an APOBEC3A deaminase (A3A) and an RNA-guided nickase;
   (b) a second mRNA comprising a second open reading frame encoding a uracil glycosylase inhibitor (UGI); and
   (c) one or more guide RNAs.
Embodiment A85. The method of embodiment A84, wherein parts (a) and (b) are in separate lipid nucleic acid assembly compositions.
Embodiment A86. The method of embodiment A84, wherein parts (a) and (b) are in the same lipid nucleic acid assembly composition.
Embodiment A87. The method of embodiment A84, wherein parts (a) and (c) are in separate lipid nucleic acid assembly compositions.
Embodiment A88. The method of embodiment A84, wherein parts (a) and (c) are in the same lipid nucleic acid assembly composition.
Embodiment A89. The method of embodiment A84, wherein parts (b) and (c) are in separate lipid nucleic acid assembly compositions.
Embodiment A90. The method of embodiment A84, wherein parts (a) and (c) are in the same lipid nucleic acid assembly composition, and part (b) is in a separate lipid nucleic acid assembly composition.
Embodiment A91. The method of embodiment A84, wherein parts (a), (b), and (c) are each in separate lipid nucleic acid assembly compositions.
Embodiment A92. The method of embodiment A84, wherein parts (a), (b), and (c) are in the same lipid nucleic acid assembly composition.
Embodiment A93. The method of any one of embodiments A84-A88, A90, and A92, wherein the one or more guide RNAs are each in separate lipid nucleic acid assembly compositions.
Embodiment A94. The method of any one of embodiments A84-A93, comprising delivering to the cell a lipid nucleic acid assembly composition comprising a first mRNA comprising a first open reading frame encoding a polypeptide comprising an APOBEC3A deaminase (A3A) and an RNA-guided nickase and a second mRNA comprising a second open reading frame encoding a uracil glycosylase inhibitor (UGI) in the same lipid nucleic acid assembly composition.
Embodiment A95. The method of any one of embodiments A84-A93, comprising delivering to the cell a first lipid nucleic acid assembly composition comprising a first mRNA comprising a first open reading frame encoding a polypeptide comprising an APOBEC3A deaminase (A3A) and an RNA-guided nickase, and a second lipid nucleic acid assembly composition comprising a second mRNA comprising a second open reading frame encoding a uracil glycosylase inhibitor (UGI).
Embodiment A96. The method of any one of embodiments A84-A95, further comprising delivering one or more guide RNAs in one or more lipid nucleic acid assembly compositions that are separate from the lipid nucleic acid assembly compositions comprising the A3A and UGI.
Embodiment A97. The method of any one of embodiments A84-A96, wherein at least 2, 3, 4, 5, 6, 7, 8, 9, or 10 lipid nucleic acid assembly compositions are delivered to the cell.
Embodiment A98. The method of any one of embodiments A84-A97, wherein at least one lipid nucleic acid assembly composition comprises lipid nanoparticle (LNPs), optionally wherein all lipid nucleic acid assembly compositions comprise LNPs.
Embodiment A99. The method of any one of embodiments A84-A98, wherein at least one lipid nucleic acid assembly composition is a lipoplex composition.
Embodiment A100. The method of any one of embodiments A84-A99, wherein the lipid nucleic acid assembly composition comprises an ionizable lipid.
Embodiment A101. The method of any one of embodiments A84-A100, wherein the lipid nucleic acid assembly composition comprises an ionizable lipid and wherein the ionizable lipid comprises a biodegradable ionizable lipid.
Embodiment A102. The method of any one of embodiments A84-A101, wherein the lipid nucleic acid assembly composition comprises an ionizable lipid and wherein the ionizable lipid has a PK value in the range of pKa in the range of from about 5.1 to about 7.4, such as from about 5.5 to about 6.6, from about 5.6 to about 6.4, from about 5.8 to about 6.2, or from about 5.8 to about 6.5.
Embodiment A103. The method of any one of embodiments A84-A102, wherein the lipid nucleic acid assembly composition comprises an amine lipid.
Embodiment A104. The method of any one of embodiments A84-A103, wherein the lipid nucleic acid assembly composition comprises Lipid A.
Embodiment A105. The method of any one of embodiments A84-A104, wherein the lipid nucleic acid assembly composition comprises a helper lipid.
Embodiment A106. The method of any one of embodiments A84-A105, wherein the lipid nucleic acid assembly composition comprises a helper lipid, wherein the helper lipid is cholesterol.
Embodiment A107. The method of any one of embodiments A84-A106, wherein the lipid nucleic acid assembly composition comprises a stealth lipid.
Embodiment A108. The method of any one of embodiments A84-A107, wherein the lipid nucleic acid assembly composition comprises a stealth lipid, wherein the stealth lipid is PEG2k-DMG.
Embodiment A109. The method of any one of embodiments A84-A108, wherein the lipid nucleic acid assembly composition comprises a neutral lipid.
Embodiment A110. The method of any one of embodiments A84-A109, wherein the lipid nucleic acid assembly composition comprises a neutral lipid, wherein the neutral lipid is DSPC.
Embodiment A111. The method of any one of embodiments A84-A110, wherein the lipid nucleic acid assembly composition comprises a neutral lipid, wherein the neutral lipid is present at about 9 mol-%.
Embodiment A112. The method of any one of embodiments A84-A111, wherein the lipid nucleic acid assembly composition comprises a stealth lipid, wherein the stealth lipid is present at about 3 mol-%.
Embodiment A113. The method of any one of embodiments A84-A112, wherein the lipid nucleic acid assembly composition comprises a helper lipid, wherein the helper lipid is present at about 38 mol-%.
Embodiment A114. The method of any one of embodiments A84-A113, wherein the N/P ratio of the lipid nucleic acid assembly composition is about 6.
Embodiment A115. The method of any one of embodiments A84-A114, wherein the lipid nucleic acid assembly composition comprises about 50 mol-% amine lipid such as Lipid A; about 9 mol-% neutral lipid such as DSPC; about 3 mol-% of stealth lipid such as a PEG lipid, such as PEG2k-DMG, and the remainder of the lipid component is helper lipid such as cholesterol wherein the N/P ratio of the is about 6.
Embodiment A116. The method of any one of embodiments A84-A115, comprising one gRNA that targets a gene that reduces or eliminates MHC class I expression on the surface of a cell, and/or one gRNA that targets a gene that reduces or eliminates MHC class II expression on the surface of a cell, and/or one gRNA that targets a gene that reduces or eliminates endogenous TCR expression.
Embodiment A117. The method of any one of embodiments A84-A115, comprising at least two gRNAs selected from: one gRNA that targets a gene that reduces or eliminates MHC class I expression on the surface of a cell, one gRNA that targets a gene that reduces or eliminates MHC class II expression on the surface of a cell, and one gRNA that targets a gene that reduces or eliminates endogenous TCR expression.
Embodiment A118. The method of any one of embodiments A84-A115, comprising one gRNA that targets a gene that reduces or eliminates MHC class I expression on the surface of a cell, one gRNA that targets a gene that reduces or eliminates MHC class II expression on the surface of a cell, and one gRNA that targets a gene that reduces or eliminates endogenous TCR expression.
Embodiment A119. The method of any one of embodiments A84-A115, comprising one gRNA selected from a gRNA that targets TRAC, TRBC, B2M, HLA-A, or CIITA.
Embodiment A120. The method of any one of embodiments A84-A116, and A119, wherein the gRNA targets TRBC, wherein the gRNA comprises a guide sequence chosen from: i) SEQ ID NOs: 706-721; ii) at least 17, 18, 19, or 20 contiguous nucleotides of a sequence selected from SEQ ID NOs: 706-721; iii) a guide sequence at least 95%, 90%, or 85% identical to a sequence selected from SEQ ID NOs: 706-721; iv) a sequence that comprises 10 contiguous nucleotides±10 nucleotides of a genomic coordinate listed in Table 5A; v) at least 17, 18, 19, or 20 contiguous nucleotides of a sequence from (iv); or vi) a guide sequence that is at least 95%, 90%, or 85% identical to a sequence selected from (v).
Embodiment A121. The method of any one of embodiments A84-A116, and A119, wherein the gRNA targets TRBC, wherein the gRNA comprises a guide sequence chosen from: i) SEQ ID NOs: 618-669; ii) at least 17, 18, 19, or 20 contiguous nucleotides of a sequence selected from SEQ ID NOs: 618-669; iii) a guide sequence at least 95%, 90%, or 85% identical to a sequence selected from SEQ ID NOs: 618-669; iv) a sequence that comprises 10 contiguous nucleotides±10 nucleotides of a genomic coordinate listed in Table 5B; v) at least 17, 18, 19, or 20 contiguous nucleotides of a sequence from (iv); or vi) a guide sequence that is at least 95%, 90%, or 85% identical to a sequence selected from (v).
Embodiment A122. The method of any one of embodiments A84-A115, comprising at least two gRNAs selected from a gRNA that targets TRAC, TRBC, or B2M, wherein the two guide RNAs do not target the same gene.
Embodiment A123. The method of any one of embodiments A84-A115, comprising at least two gRNAs selected from a gRNA that targets TRAC, TRBC, or HLA-A, wherein the two guide RNAs do not target the same gene.
Embodiment A124. The method of any one of embodiments A84-A115, comprising one guide RNA that targets TRAC, and one gRNA that targets TRBC.
Embodiment A125. The method of any one of embodiments A84-A115, comprising one guide RNA that targets B2M, and one gRNA that targets CIITA.
Embodiment A126. The method of any one of embodiments A84-A115, comprising one guide RNA that targets HLA-A, and one gRNA that targets CIITA, optionally wherein the cell is homozygous for HLA-B and homozygous for HLA-C.
Embodiment A127. The method of any one of embodiments A84-A115, comprising one guide RNA that targets TRAC, and one gRNA that targets TRBC, and one gRNA that targets B2M.
Embodiment A128. The method of any one of embodiments A84-A115, comprising one guide RNA that targets TRAC, and one gRNA that targets TRBC, and one gRNA that targets HLA-A, optionally wherein the cell is homozygous for HLA-B and homozygous for HLA-C.
Embodiment A129. The method of any one of embodiments A84-A115, comprising one guide RNA that targets TRAC, and one gRNA that targets TRBC, one gRNA that targets B2M, and one gRNA that targets CIITA.
Embodiment A130. The method of any one of embodiments A84-A115, comprising one guide RNA that targets TRAC, and one gRNA that targets TRBC, one gRNA that targets HLA-A, and one gRNA that targets CIITA, optionally wherein the cell is homozygous for HLA-B and homozygous for HLA-C.
Embodiment A131. The method of any one of the preceding embodiments, wherein the method generates a cytosine (C) to thymine (T) conversion within a target sequence.
Embodiment A132. The method of any one of the preceding embodiments, wherein the method causes at least 60% C-to-T conversion relative to the total edits in the target sequence.
Embodiment A133. The method of any one of the preceding embodiments, wherein the method causes at least 65%, 70%, 75%, 80%, 85%, 90%, 95%, 98%, or 99% C-to-T conversion relative to the total edits in the target sequence.
Embodiment A134. The method of any one of the preceding embodiments, wherein the ratio of C-to-T conversion to unintended edits is larger than 1:1.
Embodiment A135. The method of any one of the preceding embodiments, wherein the ratio of C-to-T conversion to unintended edits is from 2:1 to 99:1.
Embodiment A136. The method of any one of the preceding embodiments, wherein the ratio of C-to-T conversion to unintended edits is 2:1, 3:1, 4:1, 5:1, 6:1, 7:1, or 8:1.
Embodiment A137. The method of any one of the preceding embodiments, wherein the method causes the A3A to make a base edit corresponding to any one of positions −1 to 10 relative to the 5′ end of the guide sequence.
Embodiment A138. The method of any one of the preceding embodiments, wherein the method causes the A3A to make a base edit at a position 1, 2, 3, 4, 5, 6, 7, 8, 9, or 10 nucleotides from the 5′ end of the guide sequence.
Embodiment A139. The method of any one of the preceding embodiments, wherein the first mRNA, the second mRNA, and the guide RNA if present, delivered at a ratio of about 6:2:3 (w:w:w).
Embodiment A140. The method of any one of the preceding embodiments, wherein the mRNA, composition, or LNP is administered at a total RNA amount from 5 to 600 ng.
Embodiment A141. The method of any one of the preceding embodiments, wherein the total RNA amount is from 8 to 550 ng.
Embodiment A142. The method of any one of the preceding embodiments, wherein the total RNA amount is from 35 to 550 ng.
Embodiment A143. The method of any one of the preceding embodiments, wherein the total RNA amount is from 70 to 550 ng.
Embodiment A144. The method of any one of the preceding embodiments, wherein the total RNA amount is from 138 to 550 ng.
Embodiment A145. The method of any one of the preceding embodiments, wherein the total RNA amount is from 275 to 550 ng.
Embodiment A146. The method, cell, or engineered cell of any one of the preceding embodiments, wherein the cell is a lymphocyte.
Embodiment A147. The method, cell, or engineered cell of any one of the preceding embodiments, wherein the cell is a B lymphocyte.
Embodiment A148. The method, cell, or engineered cell of any one of the preceding embodiments, wherein the cell is a T lymphocyte.
Embodiment A149. The method or use of any one of the preceding embodiments, wherein the modification of the target gene is in vivo.
Embodiment A150. The method or use of any one of the preceding embodiments, wherein the modification of the target gene is ex vivo.
Embodiment A151. The method or use of any one of the preceding embodiments, wherein the modification of the target gene reduces or eliminates expression of the target gene.
Embodiment A152. The method or use of any one of the preceding embodiments, wherein the genome editing or modification of the target gene reduces expression of the target gene by at least 50%, 55%, 60%, 65%, 70%, 75%, 80%, 85%, 90%, or 95%.
Embodiment A153. The method or use of any one of the preceding embodiments, wherein the genome editing or modification of the target gene produces a missense mutation in the gene.
Embodiment A154. A polypeptide comprising an APOBEC3A deaminase (A3A) and an RNA-guided nickase, wherein the polypeptide does not comprise a uracil glycosylase inhibitor (UGI).
Embodiment A155. A composition comprising a first polypeptide comprising an APOBEC3A deaminase (A3A) and an RNA-guided nickase and a second polypeptide comprising a UGI, wherein the second polypeptide is different from the first polypeptide.
Embodiment A156. The polypeptide of embodiment A152 or A153, wherein the A3A is fused to the RNA-guided nickase via a peptide linker, optionally XTEN.
Embodiment A157. The polypeptide of embodiment A152 or A153, wherein the A3A is attached to a linker comprising an organic molecule, polymer, or chemical moiety.
Embodiment A158. A pharmaceutical composition comprising the mRNA, composition, or polypeptide of any of the preceding embodiments Aand a pharmaceutically acceptable carrier.
Embodiment A159. A kit comprising the mRNA, composition, or polypeptide of any of the preceding embodiments A.
Embodiment A160. The mRNA, composition, method, cell, or engineered cell of any one of the preceding embodiments, wherein the polypeptide comprising an APOBEC3A deaminase (A3A) and an RNA-guided nickase includes: the A3A, a linker, and the RNA-guided nickase in amino to carboxy terminal order.
Embodiment A161. A method of altering a DNA sequence within a TRAC gene, comprising delivering to a cell:
   (a) a gRNA comprising a guide sequence chosen from: i) SEQ ID NOs: 706-721; ii) at least 17, 18, 19, or 20 contiguous nucleotides of a sequence selected from SEQ ID NOs: 706-721; iii) a guide sequence at least 95%, 90%, or 85% identical to a sequence selected from SEQ ID NOs: 706-721; iv) a sequence that comprises 10 contiguous nucleotides±10 nucleotides of a genomic coordinate listed in Table 5A; v) at least 17, 18, 19, or 20 contiguous nucleotides of a sequence from (iv); or vi) a guide sequence that is at least 95%, 90%, or 85% identical to a sequence selected from (v); or
   (b) a nucleic acid encoding a gRNA of (a.).
Embodiment A162. A method of reducing the expression of a TRAC gene, comprising delivering to a cell:
   (a) a gRNA comprising a guide sequence chosen from: i) SEQ ID NOs: 706-721; ii) at least 17, 18, 19, or 20 contiguous nucleotides of a sequence selected from SEQ ID NOs: 706-721; iii) a guide sequence at least 95%, 90%, or 85% identical to a sequence selected from SEQ ID NOs: 706-721; iv) a sequence that comprises 10 contiguous nucleotides±10 nucleotides of a genomic coordinate listed in Table 5A; v) at least 17, 18, 19, or 20 contiguous nucleotides of a sequence from (iv); or vi) a guide sequence that is at least 95%, 90%, or 85% identical to a sequence selected from (v); or
   (b) a nucleic acid encoding a gRNA of (a.).
Embodiment A163. A method of immunotherapy comprising administering a composition comprising an engineered cell to a subject,
   wherein the cell comprises a genomic modification of at least one nucleotide within the genomic coordinates selected from:
   chr14: 22547596-22547616; chr14: 22550570-22550590; chr14: 22547763-22547783; chr14: 22550596-22550616; chr14: 22550566-22550586; chr14: 22547753-22547773; chr14: 22550601-22550621; chr14: 22550599-22550619; chr14: 22547583-22547603; chr14: 22547671-22547691; chr14: 22547770-22547790; chr14: 22547676-225476%; chr14: 22547772-22547792; chr14: 22547771-22547791; chr14: 22547733-22547753; chr14: 22547776-225477%; or
   wherein the cell is engineered by delivering to the cell:
      (a) a gRNA comprising a guide sequence chosen from: i) SEQ ID NOs: 706-721; ii) at least 17, 18, 19, or 20 contiguous nucleotides of a sequence selected from SEQ ID NOs: 706-721; iii) a guide sequence at least 95%, 90%, or 85% identical to a sequence selected from SEQ ID NOs: 706-721; iv) a sequence that comprises 10 contiguous nucleotides±10 nucleotides of a genomic coordinate listed in Table 5A; v) at least 17, 18, 19, or 20 contiguous nucleotides of a sequence from (iv); or vi) a guide sequence that is at least 95%, 90%, or 85% identical to a sequence selected from (v); or
      (b) a nucleic acid encoding a gRNA of (a.).
Embodiment A164. The method of any one of embodiments A161-163, wherein the guide sequence comprises any one of SEQ ID NOs: 706-709.
Embodiment A165. The method of any one of embodiments A161-164, wherein the guide sequence comprises any one of SEQ ID NOs: 706-708.
Embodiment A166. The method of any one of embodiments A161-165, wherein the guide sequence comprises SEQ ID NO: 706.
Embodiment A167. The method of any one of embodiments A161-165, wherein the guide sequence comprises SEQ ID NO: 707.
Embodiment A168. The method of any one of embodiments A161-165, wherein the guide sequence comprises SEQ ID NO: 708.
Embodiment A169. A method of altering a DNA sequence within a TRBC1 and/or TRBC2 gene, comprising delivering a composition to a cell, wherein the composition comprises:
   (a) a gRNA comprising a guide sequence chosen from: i) SEQ ID NOs: 618-669; ii) at least 17, 18, 19, or 20 contiguous nucleotides of a sequence selected from SEQ ID NOs: 618-669; iii) a guide sequence at least 95%, 90%, or 85% identical to a sequence selected from SEQ ID NOs: 618-669; iv) a sequence that comprises 10 contiguous nucleotides±10 nucleotides of a genomic coordinate listed in Table 5B; v) at least 17, 18, 19, or 20 contiguous nucleotides of a sequence from (iv); or vi) a guide sequence that is at least 95%, 90%, or 85% identical to a sequence selected from (v); or
   (b) a nucleic acid encoding a guide RNA of (a.).
Embodiment A170. A method of reducing the expression of a TRBC1 and/or TRBC2 gene, comprising delivering a composition to a cell, wherein the composition comprises:
   (a) a gRNA comprising a guide sequence chosen from: i) SEQ ID NOs: 618-669; ii) at least 17, 18, 19, or 20 contiguous nucleotides of a sequence selected from SEQ ID NOs: 618-669; iii) a guide sequence at least 95%, 90%, or 85% identical to a sequence selected from SEQ ID NOs: 618-669; iv) a sequence that comprises 10 contiguous nucleotides±10 nucleotides of a genomic coordinate listed in Table 5B; v) at least 17, 18, 19, or 20 contiguous nucleotides of a sequence from (iv); or vi) a guide sequence that is at least 95%, 90%, or 85% identical to a sequence selected from (v); or
   (b) a nucleic acid encoding a guide RNA of (a.).
Embodiment A171. A method of immunotherapy comprising administering a composition comprising an engineered cell to a subject,
   wherein the cell comprises a modification of at least one nucleotide within the genomic coordinates selected from: chr7: 142791757-142791777; chr7: 142801104-142801124; chr7: 142791811-142791831; chr7: 142801158-142801178; chr7: 142792728-142792748; chr7: 142791719-142791739; chr7: 142791766-142791786; chr7: 142801113-142801133; chr7: 142791928-142791948; chr7: 142801275-142801295; chr7: 142792062-142792082; chr7: 142801409-142801429; chr7: 142792713-142792733; chr7: 142802126-142802146; chr7: 142791808-142791828; chr7: 142801155-142801175; chr7: 142792003-142792023; chr7: 142801350-142801370; chr7: 142791760-142791780; chr7: 142791715-142791735; chr7: 142792781-142792801; chr7: 142792040-142792060; chr7: 142801387-142801407; chr7: 142791862-142791882; chr7: 142791716-142791736; chr7: 142791787-142791807; chr7: 142791759-142791779; chr7: 142801106-142801126; chr7: 142791807-142791827; chr7: 142801154-142801174; chr7: 142791879-142791899; chr7: 142801226-142801246; chr7: 142791805-142791825; chr7: 142791700-142791720; chr7: 142791765-142791785; chr7: 142801112-142801132; chr7: 142791820-142791840; chr7: 142791872-142791892; chr7: 142801219-142801239; chr7: 142791700-142791720; chr7: 142791806-142791826; chr7: 142801153-142801173; chr7: 142792035-142792055; chr7: 142792724-142792744; chr7: 142792754-142792774; chr7: 142791804-142791824; chr7: 142792684-142792704; chr7: 142791823-142791843; chr7: 142792728-142792748; chr7: 142792721-142792741; chr7: 142792749-142792769; chr7: 142792685-142792705; chr7: 142791816-142791836; chr7: 142801163-142801183; chr7: 142792686-142792706; chr7: 142791793-142791813; chr7: 142793110-142793130; chr7: 142791815-142791835; chr7: 142801162-142801182; chr7: 142792770-142792790; chr7: 142792047-142792067; chr7: 142801394-142801414; chr7: 142791871-142791891; chr7: 142801218-142801238; chr7: 142791894-142791914; chr7: 142792723-142792743; chr7: 142792724-142792744; chr7: 142791897-142791917; chr7: 142801244-142801264; chr7: 142792757-142792777; chr7: 142792740-142792760; chr7: 142792758-142792778; or
   wherein the cell is engineered by delivering to a cell:
      (a) a gRNA comprising a guide sequence chosen from: i) SEQ ID NOs: 618-669; ii) at least 17, 18, 19, or 20 contiguous nucleotides of a sequence selected from SEQ ID NOs: 618-669; iii) a guide sequence at least 95%, 90%, or 85% identical to a sequence selected from SEQ ID NOs: 618-669; iv) a sequence that comprises 10 contiguous nucleotides±10 nucleotides of a genomic coordinate listed in Table 5B; v) at least 17, 18, 19, or 20 contiguous nucleotides of a sequence from (iv); or vi) a guide sequence that is at least 95%, 90%, or 85% identical to a sequence selected from (v); or
      (b) a nucleic acid encoding a guide RNA of (a.).
Embodiment A172. The method of any one of embodiments A169-171, wherein the guide sequence comprises any one of SEQ ID NOs: 618-627.
Embodiment A173. The method of any one of embodiments A169-172, wherein the guide sequence comprises any one of SEQ ID NOs: 618-621.
Embodiment A174. The method of any one of embodiments A169-173, wherein the guide sequence comprises SEQ ID NO: 618.
Embodiment A175. The method of any one of embodiments A169-173, wherein the guide sequence comprises SEQ ID NO: 619.
Embodiment A176. The method of any one of embodiments A169-173, wherein the guide sequence comprises SEQ ID NO: 620.
Embodiment A177. The method of any one of embodiments A169-173, wherein the guide sequence comprises SEQ ID NO: 621.
Embodiment A178. The method of any one of embodiments A161-177, wherein the composition further comprises the mRNA or composition of any one of embodiments A1-76.
Embodiment A179. A composition comprising:
   (a) a gRNA comprising a guide sequence chosen from: i) SEQ ID NOs: 706-721; ii) at least 17, 18, 19, or 20 contiguous nucleotides of a sequence selected from SEQ ID NOs: 706-721; iii) a guide sequence at least 95%, 90%, or 85% identical to a sequence selected from SEQ ID NOs: 706-721; iv) a sequence that comprises 10 contiguous nucleotides±10 nucleotides of a genomic coordinate listed in Table 5A; v) at least 17, 18, 19, or 20 contiguous nucleotides of a sequence from (iv); or vi) a guide sequence that is at least 95%, 90%, or 85% identical to a sequence selected from (v); and optionally
   (b) the mRNA or composition of any one of embodiments A1-A76.
Embodiment A180. The composition of embodiment A179, wherein the guide sequence comprises any one of SEQ ID NOs: 706-709.
Embodiment A181. The composition of embodiment A179 or A180, for use in altering a DNA sequence within the TRAC gene in a cell.
Embodiment A182. The composition of any one of embodiments A179-A181 for use in reducing the expression of the TRAC gene in a cell.
Embodiment A183. The composition of any one of embodiments A179-A182, for use in immunotherapy of a subject.
Embodiment A184. A composition comprising:
   (a) a gRNA comprising a guide sequence chosen from: i) SEQ ID NOs: 618-669; ii) at least 17, 18, 19, or 20 contiguous nucleotides of a sequence selected from SEQ ID NOs: 618-669; iii) a guide sequence at least 95%, 90%, or 85% identical to a sequence selected from SEQ ID NOs: 618-669; iv) a sequence that comprises 10 contiguous nucleotides±10 nucleotides of a genomic coordinate listed in Table 5B; v) at least 17, 18, 19, or 20 contiguous nucleotides of a sequence from (iv); or vi) a guide sequence that is at least 95%, 90%, or 85% identical to a sequence selected from (v); and optionally
   (b) the mRNA or composition of any one of embodiments A11-A75.
Embodiment A185. The composition of embodiment A184, wherein the guide sequence comprises any one of SEQ ID NOs: 618-621.
Embodiment A186. The composition of embodiment A184 or A185, for use in altering a DNA sequence within the TRBC1 and/or TRBC2 gene in a cell.
Embodiment A187. The composition of any one of embodiments A184 to A186 for use in reducing the expression of the TRBC1 and/or TRBC2 gene in a cell.
Embodiment A188. The composition of any one of embodiments A184 to A187, for use in immunotherapy of a subject.
Embodiment A189. A cell, altered by the method of any one of embodiments A120-A121, and A161-A178.
Embodiment A190. The cell according to embodiment A189, wherein the cell is altered ex vivo.
Embodiment A191. The cell according to embodiment A189 or 190, wherein the cell is a T cell.
Embodiment A192. The cell according to any of embodiments A189-A191, wherein the cell is a CD4⁺ or CD8⁺ T cell.
Embodiment A193. The cell according to any of embodiments A189-A192, wherein the cell is a mammalian, primate, or human cell.
Embodiment A194. The cell according to any of embodiments A189-A193, for use in immunotherapy of a subject.
Embodiment A195. An engineered cell which has reduced or eliminated surface expression of TRAC, comprising a genetic modification in a human TRAC gene, wherein the genetic modification comprises a modification of at least one nucleotide within the genomic coordinates selected from:
   chr14: 22547596-22547616; chr14: 22550570-22550590; chr14: 22547763-22547783; chr14: 22550596-22550616; chr14: 22550566-22550586; chr14: 22547753-22547773; chr14: 22550601-22550621; chr14: 22550599-22550619; chr14: 22547583-22547603; chr14: 22547671-22547691; chr14: 22547770-22547790; chr14: 22547676-22547696; chr14: 22547772-22547792; chr14: 22547771-22547791; chr14: 22547733-22547753; chr14: 22547776-22547796.
Embodiment A196. An engineered cell which has reduced or eliminated surface expression of TRBC1/2, comprising a genetic modification in a human TRBC1/2 gene, wherein the genetic modification comprises a modification of at least one nucleotide within the genomic coordinates selected from:
   chr7: 142791757-142791777; chr7: 142801104-142801124; chr7: 142791811-142791831; chr7: 142801158-142801178; chr7: 142792728-142792748; chr7: 142791719-142791739; chr7: 142791766-142791786; chr7: 142801113-142801133; chr7: 142791928-142791948; chr7: 142801275-142801295; chr7: 142792062-142792082; chr7: 142801409-142801429; chr7: 142792713-142792733; chr7: 142802126-142802146; chr7: 142791808-142791828; chr7: 142801155-142801175; chr7: 142792003-142792023; chr7: 142801350-142801370; chr7: 142791760-142791780; chr7: 142791715-142791735; chr7: 142792781-142792801; chr7: 142792040-142792060; chr7: 142801387-142801407; chr7: 142791862-142791882; chr7: 142791716-142791736; chr7: 142791787-142791807; chr7: 142791759-142791779; chr7: 142801106-142801126; chr7: 142791807-142791827; chr7: 142801154-142801174; chr7: 142791879-142791899; chr7: 142801226-142801246; chr7: 142791805-142791825; chr7: 142791700-142791720; chr7: 142791765-142791785; chr7: 142801112-142801132; chr7: 142791820-142791840; chr7: 142791872-142791892; chr7: 142801219-142801239; chr7: 142791700-142791720; chr7: 142791806-142791826; chr7: 142801153-142801173; chr7: 142792035-142792055; chr7: 142792724-142792744; chr7: 142792754-142792774; chr7: 142791804-142791824; chr7: 142792684-142792704; chr7: 142791823-142791843; chr7: 142792728-142792748; chr7: 142792721-142792741; chr7: 142792749-142792769; chr7: 142792685-142792705; chr7: 142791816-142791836; chr7: 142801163-142801183; chr7: 142792686-142792706; chr7: 142791793-142791813; chr7: 142793110-142793130; chr7: 142791815-142791835; chr7: 142801162-142801182; chr7: 142792770-142792790; chr7: 142792047-142792067; chr7: 142801394-142801414; chr7: 142791871-142791891; chr7: 142801218-142801238; chr7: 142791894-142791914; chr7: 142792723-142792743; chr7: 142792724-142792744; chr7: 142791897-142791917; chr7: 142801244-142801264; chr7: 142792757-142792777; chr7: 142792740-142792760; chr7: 142792758-142792778.
Embodiment A197. One or more lipid nucleic acid assembly compositions, optionally lipid nanoparticles, comprising:
   (a) a first mRNA comprising a first open reading frame encoding a polypeptide comprising an APOBEC3A deaminase (A3A) and an RNA-guided nickase;
   (b) a second mRNA comprising a second open reading frame encoding a uracil glycosylase inhibitor (UGI); and
   (c) one or more guide RNAs.
Embodiment A198. The method, cell, or engineered cell of any one of the preceding embodiments, wherein the cell is an immune cell.
Embodiment A199. The method, cell, or engineered cell of any one of the preceding embodiments, wherein the cell is a lymphocyte.
Embodiment A200. The method, cell, or engineered cell of any one of the preceding embodiments, wherein the cell is a T cell.

The following numbered embodiments provide additional support for and descriptions of the embodiments herein.
Embodiment B1. An mRNA comprising an open reading frame encoding a polypeptide comprising a cytidine deaminase and an RNA-guided nickase, wherein the polypeptide does not comprise a uracil glycosylase inhibitor (UGI).
Embodiment B2. A composition comprising a first mRNA comprising a first open reading frame encoding a polypeptide comprising a cytidine deaminase and an RNA-guided nickase, and a second mRNA comprising a second open reading frame encoding a uracil glycosylase inhibitor (UGI), wherein the second mRNA is different from the first mRNA, optionally wherein the composition comprises lipid nanoparticles.
Embodiment B3. The composition of embodiment B2, wherein the first open reading frame does not comprise a sequence encoding a UGI.
Embodiment B4. The composition of embodiment B2 or B3, wherein the molar ratio of the second mRNA to the first mRNA is from 1:1 to 30:1.
Embodiment B5. The composition of any one of embodiments B2-B4, wherein the composition comprises a first composition and a second composition, wherein the first composition comprises a first mRNA comprising a first open reading frame encoding a polypeptide comprising a cytidine deaminase and an RNA-guided nickase and does not comprise a uracil glycosylase inhibitor (UGI), and the second composition comprises a second mRNA comprising a second open reading frame encoding a uracil glycosylase inhibitor (UGI), wherein the second mRNA is different from the first mRNA, optionally wherein the compositions comprise lipid nanoparticles.
Embodiment B6. The composition of any one of embodiments B2-B5, wherein the first mRNA and the second mRNAs are in the same or separate vials.
Embodiment B7. A method of modifying a target gene comprising delivering to a cell a first mRNA comprising a first open reading frame encoding a first polypeptide comprising a cytidine deaminase and an RNA-guided nickase, a second mRNA comprising a second open reading frame encoding a uracil glycosylase inhibitor (UGI), wherein the second mRNA is different from the first mRNA, and at least one guide RNA (gRNA), wherein if the nickase is a SpyCas9 nickase, then the gRNA is a SpyCas9 gRNA, and if the nickase is a NmeCas9 nickase, then the gRNA is a Nme gRNA.
Embodiment B8. The composition or method of any one of embodiments B2-B7, wherein the molar ratio of the second mRNA to the first mRNA is from 1:1 to 30:1.
Embodiment B9. The composition or method of any one of embodiments B2-B7, wherein the molar ratio is from 2:1 to 30:1.
Embodiment B10. The composition or method of any one of embodiments B2-B7, wherein the molar ratio is from 7:1 to 22:1.
Embodiment B11. A cell comprising a composition comprising a first mRNA comprising a first open reading frame encoding a polypeptide comprising a cytidine deaminase and an RNA-guided nickase, and a second mRNA comprising a second open reading frame encoding a uracil glycosylase inhibitor (UGI), wherein the second mRNA is different from the first mRNA.
Embodiment B12. An engineered cell comprising at least one base edit and/or indel, wherein the base edit and/or indel is made by contacting a cell with a composition comprising a first mRNA comprising a first open reading frame encoding a polypeptide comprising a cytidine deaminase and an RNA-guided nickase, and a second mRNA comprising a second open reading frame encoding a uracil glycosylase inhibitor (UGI), wherein the second mRNA is different from the first mRNA.
Embodiment B13. The mRNA, composition, method, cell, or engineered cell of any one of the preceding embodiments, wherein the cytidine deaminase is
(i) an enzyme of APOBEC family, optionally an enzyme of APOBEC3 subgroup;
(ii) a cytidine deaminase comprising an amino acid sequence that is at least 80% identical to any one of SEQ ID NOs: 40, 41, and 960-1023;
(iii) a cytidine deaminase comprising an amino acid sequence that is at least 80% identical to any one of SEQ ID NOs: 40, 41, and 960-1013;
(iv) a cytidine deaminase comprising an amino acid sequence that is at least 80% identical to any one of SEQ ID NOs: 40, 41, 976, 977, 979, 980, 984-987, 993-1006, and 1009; or
(v) a cytidine deaminase comprising an amino acid sequence that is at least 80% identical to any one of SEQ ID NOs: 40, 976, 981, 984, 986, and 1014-1023.
Embodiment B314. The mRNA, composition, method, cell, or engineered cell of embodiment B13, wherein the cytidine deaminase comprises an amino acid sequence with at least 80%, 85%, 87%, 90%, 95%, 98%, 99%, or 100% identity to SEQ ID NO: 40, 41, and 960-1023.
Embodiment B15. The mRNA, composition, method, cell, or engineered cell of embodiment B13, wherein the cytidine deaminase comprises an amino acid sequence with at least 80%, 85%, 87%, 90%, 95%, 98%, 99%, or 100% identity to SEQ ID NO: 40, 41, and 960-1013.
Embodiment B16. The mRNA, composition, method, cell, or engineered cell of embodiment B13, wherein the cytidine deaminase comprises an amino acid sequence with at least 80%, 85%, 87%, 90%, 95%, 98%, 99%, or 100% identity to SEQ ID NO: 40, 41, 976, 977, 979, 980, 984-987, 993-1006, and 1009.
Embodiment B17. The mRNA, composition, method, cell, or engineered cell of embodiment B13, wherein the cytidine deaminase comprises an amino acid sequence with at least 80%, 85%, 87%, 90%, 95%, 98%, 99%, or 100% identity to SEQ ID NO: 40, 976, 981, 984, 986, 1014-1023.
Embodiment B18. The mRNA, composition, method, cell, or engineered cell of any one of the preceding embodiments, wherein the cytidine deaminase is an APOBEC3A deaminase (A3A).
Embodiment B19. The mRNA, composition, method, cell, or engineered cell of embodiment B18, wherein the A3A comprises an amino acid sequence with at least 87% identity to SEQ ID NO: 40.
Embodiment B20. The mRNA, composition, method, cell, or engineered cell of embodiment B18, wherein the A3A comprises an amino acid sequence with at least 90% identity to SEQ ID NO: 40.
Embodiment B21. The mRNA, composition, method, cell, or engineered cell of embodiment B18, wherein the A3A comprises an amino acid sequence with at least 95% identity to SEQ ID NO: 40.
Embodiment B22. The mRNA, composition, method, cell, or engineered cell of embodiment B18, wherein the A3A comprises an amino acid sequence with at least 98% identity to SEQ ID NO: 40.
Embodiment B23. The mRNA, composition, method, cell, or engineered cell of embodiment B18, wherein the A3A comprises an amino acid sequence with at least 99% identity to SEQ ID NO: 40.
Embodiment B24. The mRNA, composition, method, cell, or engineered cell of embodiment B18, wherein the A3A comprises the amino acid sequence of SEQ ID NO: 40.
Embodiment B25. The mRNA, composition, method, cell, or engineered cell of any one of embodiments B18-B24, wherein the A3A is a human A3A.
Embodiment B26. The mRNA, composition, method, cell, or engineered cell of any one of embodiments B18-B24, wherein the A3A is a wild-type A3A.
Embodiment B27. The mRNA, composition, method, cell, or engineered cell of embodiment B18, wherein the A3A comprises an amino acid sequence with at least 87%, 90%, 95%, 98%, 99%, or 100% identity to SEQ ID NO: 976, 977, 993-1006, and 1009.
Embodiment B28. The composition, method, cell, or engineered cell of any one of the preceding embodiments, wherein the UGI comprises an amino acid sequence with at least 80% identity to SEQ ID NO: 27.
Embodiment B29. The composition, method, cell, or engineered cell of any one of the preceding embodiments, wherein the UGI comprises an amino acid sequence with at least 90% identity to SEQ ID NO: 27.
Embodiment B30. The composition, method, cell, or engineered cell of any one of the preceding embodiments, wherein the UGI comprises an amino acid sequence with at least 95% identity to SEQ ID NO: 27.
Embodiment B31. The composition, method, cell, or engineered cell of any one of the preceding embodiments, wherein the UGI comprises an amino acid sequence with at least 98% identity to SEQ ID NO: 27.
Embodiment B32. The composition, method, cell, or engineered cell of any one of the preceding embodiments, wherein the UGI comprises an amino acid sequence with at least 99% identity to SEQ ID NO: 27.
Embodiment B33. The composition, method, cell, or engineered cell of any one of the preceding embodiments, wherein the UGI comprises the amino acid sequence of SEQ ID NO: 27.
Embodiment B34. The composition, method, or cell of any one of the preceding embodiments, further comprising at least one guide RNA (gRNA).
Embodiment B35. The composition, method, cell, or engineered cell of any one of the preceding embodiments, comprising a gRNA, wherein the gRNA is an sgRNA.
Embodiment B36. The composition, method, cell, or engineered cell of any one of the preceding embodiments, comprising a gRNA, wherein the gRNA is a dgRNA.
Embodiment B37. The composition, method, cell, or engineered cell of any one of the preceding embodiments, comprising a gRNA, wherein the gRNA is a short-single guide RNA (short-sgRNA) comprising a conserved portion of an sgRNA comprising a hairpin region, wherein the hairpin region lacks at least 5-10 nucleotides and wherein the short-sgRNA comprises a 5′ end modification or a 3′ end modification or both.
Embodiment B38. The mRNA, composition, method, cell, or engineered cell of any one of the preceding embodiments, wherein the RNA-guided nickase is a Cas nickase.
Embodiment B39. The mRNA, composition, method, cell, or engineered cell of any one of the preceding embodiments, wherein the RNA-guided nickase is a Class 2 Cas nickase.
Embodiment B40. The mRNA, composition, method, cell, or engineered cell of any one of the preceding embodiments, wherein the RNA-guided nickase is a Cas9 nickase.
Embodiment B41. The mRNA, composition, method, cell, or engineered cell of any one of the preceding embodiments, wherein the RNA-guided nickase is an S. pyogenes (Spy) Cas9 nickase.
Embodiment B42. The mRNA, composition, method, cell, or engineered cell of embodiment B41, wherein the RNA-guided nickase is a D10A SpyCas9 nickase.
Embodiment B43. The mRNA, composition, method, cell, or engineered cell of any one of the preceding embodiments, wherein the RNA-guided nickase comprises an amino acid sequence having at least 80%, 90%, 95%, 98%, or 99% identity to any one of SEQ ID NOs: 70, 73, or 76.
Embodiment B44. The mRNA, composition, method, cell, or engineered cell of any one of the preceding embodiments, wherein the RNA-guided nickase comprises the amino acid sequence of any one of SEQ ID NOs: 70, 73, or 76.
Embodiment B45. The mRNA, composition, method, cell, or engineered cell of any one of the preceding embodiments, wherein the sequence encoding the RNA-guided nickase comprises a nucleotide sequence having at least 80%, 90%, 95%, 98%, or 99% identity to the nucleotide sequence of any one of SEQ ID NOs: 72, 75, or 78.
Embodiment B46. The mRNA, composition, method, cell, or engineered cell of any one of the preceding embodiments, wherein the sequence encoding the RNA-guided nickase comprises a nucleotide sequence having at least 90% to the nucleotide sequence of any one of SEQ ID NOs: 72, 75, or 78.
Embodiment B47. The mRNA, composition, method, cell, or engineered cell of any one of the preceding embodiments, wherein the sequence encoding the RNA-guided nickase comprises a nucleotide sequence having at least 95% to the nucleotide sequence of any one of SEQ ID NOs: 72, 75, or 78.
Embodiment B48. The mRNA, composition, method, cell, or engineered cell of any one of the preceding embodiments, wherein the sequence encoding the RNA-guided nickase comprises a nucleotide sequence having at least 98% to the nucleotide sequence of any one of SEQ ID NOs: 72, 75, or 78.
Embodiment B49. The mRNA, composition, method, cell, or engineered cell of any one of the preceding embodiments, wherein the sequence encoding the RNA-guided nickase comprises a nucleotide sequence having at least 99% to the nucleotide sequence of any one of SEQ ID NOs: 72, 75, or 78.
Embodiment B50. The mRNA, composition, method, cell, or engineered cell of any one of the preceding embodiments, wherein the sequence encoding the RNA-guided nickase comprises the nucleotide sequence of any one of SEQ ID NOs: 71, 72, 74, 75, or 77-90.
Embodiment B51. The mRNA, composition, method, cell, or engineered cell of any one of embodiments B1-37, wherein the RNA-guided nickase is a N. meningitidis (Nme) Cas9 nickase.
Embodiment B52. The mRNA, composition, method, cell, or engineered cell of embodiment B51, wherein the RNA-guided nickase is a D16A NmeCas9 nickase, optionally a D16A Nme2Cas9.
Embodiment B53. The mRNA, composition, method, cell, or engineered cell of any one of embodiments B1-B37, B51, or B52, wherein the sequence encoding the RNA-guided nickase comprises the nucleotide sequence of any one of SEQ ID NOs: 380 and 387.
Embodiment B54. The mRNA, composition, method, cell, or engineered cell of any one of the preceding embodiments, wherein the mRNA comprises a 5′ UTR with at least 90% identity to any one of SEQ ID NOs: 91-98.
Embodiment B55. The mRNA, composition, method, cell, or engineered cell of any one of the preceding embodiments, wherein the mRNA comprises a 3′ UTR with at least 90% identity to any one of SEQ ID NOs: 99-106.
Embodiment B56. The mRNA, composition, method, cell, or engineered cell of any one of the preceding embodiments, wherein the mRNA comprises a 5′ UTR and a 3′ UTR from the same source.
Embodiment B57. The mRNA, composition, method, cell, or engineered cell of any one of the preceding embodiments, wherein the mRNA comprises a 5′ cap selected from Cap0, Cap1, and Cap2.
Embodiment B58. The mRNA, composition, method, cell, or engineered cell of any one of the preceding embodiments, wherein the open reading frame encoding a polypeptide comprising a cytidine deaminase and an RNA-guided nickase, and/or the open reading frame encoding a uracil glycosylase inhibitor (UGI) comprise minimal adenine codons and/or minimal uridine codons.
Embodiment B59. The mRNA, composition, method, cell, or engineered cell of any one of the preceding embodiments, wherein the open reading frame encoding a polypeptide comprising a cytidine deaminase and an RNA-guided nickase, and/or the open reading frame encoding a uracil glycosylase inhibitor (UGI) comprise minimal adenine codons.
Embodiment B60. The mRNA, composition, method, cell, or engineered cell of any one of the preceding embodiments, wherein the open reading frame encoding a polypeptide comprising a cytidine deaminase and an RNA-guided nickase, and/or the open reading frame encoding a uracil glycosylase inhibitor (UGI) have codons that increase translation of the mRNA in a mammal.
Embodiment B61. The mRNA, composition, method, cell, or engineered cell of any one of the preceding embodiments, wherein the open reading frame encoding a polypeptide comprising a cytidine deaminase and an RNA-guided nickase, and/or the open reading frame encoding a uracil glycosylase inhibitor (UGI) have codons that increase translation of the mRNA in a mammal, wherein the mammal is a human.
Embodiment B62. The mRNA, composition, method, cell, or engineered cell of any one of the preceding embodiments, wherein the cytidine deaminase is located N-terminal to the RNA-guided nickase in the polypeptide.
Embodiment B63. The mRNA, composition, method, cell, or engineered cell of any one of the preceding embodiments, wherein the encoded RNA-guided nickase comprises a nuclear localization signal (NLS).
Embodiment B64. The mRNA, composition, method, cell, or engineered cell of any one of the preceding embodiments, wherein the encoded RNA-guided nickase comprises a nuclear localization signal (NLS), and wherein the NLS is at the C-terminus of the RNA-guided nickase.
Embodiment B65. The mRNA, composition, method, cell, or engineered cell of any one of the preceding embodiments, wherein the encoded RNA-guided nickase comprises a nuclear localization signal (NLS), and wherein the NLS is at the N-terminus of the RNA-guided nickase, or wherein an NLS is fused to both the N-terminus and C-terminus of the RNA-guided nickase.
Embodiment B66. The mRNA, composition, method, cell, or engineered cell of any one of the preceding embodiments, wherein the encoded RNA-guided nickase comprises a nuclear localization signal (NLS), and wherein a linker is present between the N-terminus of the RNA-guided nickase and the NLS, optionally wherein the linker is a peptide linker.
Embodiment B67. The mRNA, composition, method, cell, or engineered cell of any one of the preceding embodiments, wherein the encoded RNA-guided nickase comprises a nuclear localization signal (NLS), and wherein the NLS comprises a sequence having at least 80%, 85%, 90%, or 95% identity to any one of SEQ ID NOs: 63 and 110-122.
Embodiment B68. The mRNA, composition, method, cell, or engineered cell of any one of the preceding embodiments, wherein the encoded RNA-guided nickase comprises a nuclear localization signal (NLS), and wherein the NLS comprises the sequence of any one of SEQ ID NOs: 63 and 110-122.
Embodiment B69. The mRNA, composition, method, cell, or engineered cell of any one of the preceding embodiments, wherein the encoded RNA-guided nickase comprises a nuclear localization signal (NLS), and wherein the NLS is encoded by a sequence having at least 80%, 85%, 90%, 95%, 98% or 100% identity to the sequence of any one of SEQ ID NOs: 123-135.
Embodiment B70. The mRNA, composition, method, cell, or engineered cell of any one of the preceding embodiments, wherein the encoded RNA-guided nickase comprises a nuclear localization signal (NLS), and wherein the cytidine deaminase is located N-terminal to the NLS in the polypeptide.
Embodiment B71. The mRNA, composition, method, cell, or engineered cell of any one of the preceding embodiments, wherein the encoded RNA-guided nickase comprises a nuclear localization signal (NLS), and wherein the RNA-guided nickase is located N-terminal to the NLS in the polypeptide.
Embodiment B72. The mRNA, composition, method, cell, or engineered cell of any one of the preceding embodiments, wherein the open reading frame encoding a polypeptide comprising a cytidine deaminase and an RNA-guided nickase comprises a sequence having at least 80%, 85%, 90%, 95%, 98% or 100% identity to the sequence of SEQ ID NO:1.
Embodiment B73. The mRNA, composition, method, cell, or engineered cell of any one of the preceding embodiments, wherein the open reading frame encoding a polypeptide comprising a cytidine deaminase and an RNA-guided nickase comprises a sequence having at least 80%, 85%, 90%, 95%, 98% or 100% identity to the sequence of SEQ ID NO: 4.
Embodiment B74. The mRNA, composition, method, cell, or engineered cell of any one of the preceding embodiments, wherein at least 10% of the uridine in the mRNA is substituted with a modified uridine.
Embodiment B75. The mRNA, composition, method, cell or engineered cell of embodiment B64, wherein the modified uridine is one or more of N1-methyl-pseudouridine, pseudouridine, 5-methoxyuridine, or 5-iodouridine.
Embodiment B76. The mRNA, composition, method, cell, or engineered cell of embodiment B64 or 65, wherein the modified uridine is one or both of N1-methyl-pseudouridine or 5-methoxyuridine.
Embodiment B77. The mRNA, composition, method, cell, or engineered cell of any one of embodiments B74-B76, wherein the modified uridine is N1-methyl-pseudouridine.
Embodiment B78. The mRNA, composition, method, cell, or engineered cell of any one of embodiments B74-B76, wherein the modified uridine is 5-methoxyuridine.
Embodiment B79. The mRNA, composition, method, cell, or engineered cell of any one of embodiments B74-B78, wherein 15% to 45% of the uridine is substituted with the modified uridine.
Embodiment B80. The mRNA, composition, method, cell, or engineered cell of any one of embodiments B74-B79, wherein at least 20% or at least 30% of the uridine is substituted with the modified uridine.
Embodiment B81. The mRNA, composition, method, cell, or engineered cell of any one of embodiments B74-B80, wherein at least 80% or at least 90% of the uridine is substituted with the modified uridine.
Embodiment B82. The mRNA, composition, method, cell, or engineered cell of any one of embodiments B74-B81, wherein 100% uridine is substituted with the modified uridine.
Embodiment B83. The mRNA, composition, method, or cell of any one of the preceding embodiments, further encoding a peptide linker between the cytidine deaminase and RNA-guided nickase, optionally wherein the peptide linker is XTEN.
Embodiment B84. The mRNA, composition, method, or cell of any one of the preceding embodiments, further encoding a peptide linker between the cytidine deaminase and RNA-guided nickase, wherein the peptide linker comprises at least 1, at least 2, at least 3, at least 4, at least 5, at least 6, at least 7, at least 8, at least 9, at least 10, at least 15, at least 20, at least 25, at least 30, at least 40, at least 50, or more amino acids.
Embodiment B85. The mRNA, composition, method, or cell of any one of the preceding embodiments, further encoding a peptide linker between the cytidine deaminase and RNA-guided nickase, wherein the peptide linker comprises one or more sequences selected from SEQ ID NOs: 46-59, 61 and 211-272.
Embodiment B86. A polypeptide encoded by the mRNA of any one of the preceding embodiments.
Embodiment B87. A ribonucleoprotein complex (RNP) comprising (i) a polypeptide encoded by any one the mRNAs of any one of the preceding embodiments; and (ii) a guide RNA.
Embodiment B88. A vector comprising the mRNA of any one of the preceding embodiments.
Embodiment B89. An expression construct comprising a promoter operably linked to a sequence encoding the mRNA of any one of the preceding embodiments.
Embodiment B90. A plasmid comprising the expression construct of embodiment B89.
Embodiment B91. A host cell comprising the vector of embodiment B88, the expression construct of embodiment B89, or the plasmid of embodiment B90.
Embodiment B92. The mRNA or composition of any one of the preceding embodiments, wherein the mRNA or composition is formulated as a lipid nucleic acid assembly composition, optionally a lipid nanoparticle.
Embodiment B93. Use of the mRNA or composition according to any one of the preceding embodiments for modifying a target gene in a cell.
Embodiment B94. Use of the mRNA or composition according to any one of the preceding embodiments for the manufacture of a medicament for modifying a target gene in a cell.
Embodiment B95. A method of modifying a target gene in a cell, comprising delivering to the cell one or more lipid nucleic acid assembly compositions, optionally lipid nanoparticles, comprising:
(a) a first mRNA comprising a first open reading frame encoding a polypeptide comprising a cytidine deaminase and an RNA-guided nickase;
(b) a second mRNA comprising a second open reading frame encoding a uracil glycosylase inhibitor (UGI); and
(c) one or more guide RNAs.
Embodiment B96. The method of embodiment B95, wherein parts (a) and (b) are in separate lipid nucleic acid assembly compositions.
Embodiment B97. The method of embodiment B95, wherein parts (a) and (b) are in the same lipid nucleic acid assembly composition.
Embodiment B98. The method of embodiment B95, wherein parts (a) and (c) are in separate lipid nucleic acid assembly compositions.
Embodiment B99. The method of embodiment B95, wherein parts (a) and (c) are in the same lipid nucleic acid assembly composition.
Embodiment B3100. The method of embodiment B95, wherein parts (b) and (c) are in separate lipid nucleic acid assembly compositions.
Embodiment B101. The method of embodiment B95, wherein parts (a) and (c) are in the same lipid nucleic acid assembly composition, and part (b) is in a separate lipid nucleic acid assembly composition.
Embodiment B102. The method of embodiment B95, wherein parts (a), (b), and (c) are each in separate lipid nucleic acid assembly compositions.
Embodiment B103. The method of embodiment B95, wherein parts (a), (b), and (c) are in the same lipid nucleic acid assembly composition.
Embodiment B104. The method of any one of embodiments B95, and B98-B103, wherein the one or more guide RNAs are each in separate lipid nucleic acid assembly compositions.
Embodiment B105. The method of any one of embodiments B95-B104, comprising delivering to the cell a lipid nucleic acid assembly composition comprising a first mRNA comprising a first open reading frame encoding a polypeptide comprising a cytidine deaminase and an RNA-guided nickase and a second mRNA comprising a second open reading frame encoding a uracil glycosylase inhibitor (UGI) in the same lipid nucleic acid assembly composition.
Embodiment B106. The method of any one of embodiments B95-B104, comprising delivering to the cell a first lipid nucleic acid assembly composition comprising a first mRNA comprising a first open reading frame encoding a polypeptide comprising a cytidine deaminase and an RNA-guided nickase, and a second lipid nucleic acid assembly composition comprising a second mRNA comprising a second open reading frame encoding a uracil glycosylase inhibitor (UGI).
Embodiment B107. The method of any one of embodiments B95-B104, further comprising delivering one or more guide RNAs in one or more lipid nucleic acid assembly compositions that are separate from the lipid nucleic acid assembly compositions comprising the cytidine deaminase and UGI.
Embodiment B108. The method of any one of embodiments B95-B107, wherein at least 2, 3, 4, 5, 6, 7, 8, 9, or 10 lipid nucleic acid assembly compositions are delivered to the cell.
Embodiment B109. The method of any one of embodiments B95-B108, wherein at least one lipid nucleic acid assembly composition comprises lipid nanoparticle (LNPs), optionally wherein all lipid nucleic acid assembly compositions comprise LNPs.
Embodiment B3110. The method of any one of embodiments B95-B109, wherein at least one lipid nucleic acid assembly composition is a lipoplex composition.
Embodiment B111. The method of any one of embodiments B95-B110, wherein the lipid nucleic acid assembly composition comprises an ionizable lipid.
Embodiment B112. The method of any one of embodiments B95-B111, wherein the lipid nucleic acid assembly composition comprises an ionizable lipid and wherein the ionizable lipid comprises a biodegradable ionizable lipid.
Embodiment B113. The method of any one of embodiments B95-B112, wherein the lipid nucleic acid assembly composition comprises an ionizable lipid and wherein the ionizable lipid has a PK value in the range of pKa in the range of from about 5.1 to about 7.4, such as from about 5.5 to about 6.6, from about 5.6 to about 6.4, from about 5.8 to about 6.2, or from about 5.8 to about 6.5.
Embodiment B114. The method of any one of embodiments B95-B113, wherein the lipid nucleic acid assembly composition comprises an amine lipid.
Embodiment B115. The method of any one of embodiments B95-B114, wherein the lipid nucleic acid assembly composition comprises Lipid A.
Embodiment B116. The method of any one of embodiments B95-B115, wherein the lipid nucleic acid assembly composition comprises a helper lipid.
Embodiment B117. The method of any one of embodiments B95-B116, wherein the lipid nucleic acid assembly composition comprises a helper lipid, wherein the helper lipid is cholesterol.
Embodiment B118. The method of any one of embodiments B95-B117, wherein the lipid nucleic acid assembly composition comprises a stealth lipid.
Embodiment B119. The method of any one of embodiments B95-B118, wherein the lipid nucleic acid assembly composition comprises a stealth lipid, wherein the stealth lipid is PEG2k-DMG.
Embodiment B120. The method of any one of embodiments B95-B119, wherein the lipid nucleic acid assembly composition comprises a neutral lipid.
Embodiment B121. The method of any one of embodiments B95-B120, wherein the lipid nucleic acid assembly composition comprises a neutral lipid, wherein the neutral lipid is DSPC.
Embodiment B122. The method of any one of embodiments B95-B121, wherein the lipid nucleic acid assembly composition comprises a neutral lipid, wherein the neutral lipid is present at about 9 mol-%.
Embodiment B123. The method of any one of embodiments B95-B122, wherein the lipid nucleic acid assembly composition comprises a stealth lipid, wherein the stealth lipid is present at about 3 mol-%.
Embodiment B124. The method of any one of embodiments B95-B123, wherein the lipid nucleic acid assembly composition comprises a helper lipid, wherein the helper lipid is present at about 38 mol-%.
Embodiment B125. The method of any one of embodiments B95-B124, wherein the N/P ratio of the lipid nucleic acid assembly composition is about 6.
Embodiment B126. The method of any one of embodiments B95-B125, wherein the lipid nucleic acid assembly composition comprises about 50 mol-% amine lipid such as Lipid A; about 9 mol-% neutral lipid such as DSPC; about 3 mol-% of stealth lipid such as a PEG lipid, such as PEG2k-DMG, and the remainder of the lipid component is helper lipid such as cholesterol wherein the N/P ratio of the is about 6.
Embodiment B127. The method of any one of embodiments B95-B126, comprising one gRNA that targets a gene that reduces or eliminates MHC class I expression on the surface of a cell, and/or one gRNA that targets a gene that reduces or eliminates MHC class II expression on the surface of a cell, and/or one gRNA that targets a gene that reduces or eliminates endogenous TCR expression.
Embodiment B128. The method of any one of embodiments B95-B127, comprising at least two gRNAs selected from: one gRNA that targets a gene that reduces or eliminates MHC class I expression on the surface of a cell, one gRNA that targets a gene that reduces or eliminates MHC class II expression on the surface of a cell, and one gRNA that targets a gene that reduces or eliminates endogenous TCR expression.
Embodiment B129. The method of any one of embodiments B95-B128, comprising one gRNA that targets a gene that reduces or eliminates MHC class I expression on the surface of a cell, one gRNA that targets a gene that reduces or eliminates MHC class II expression on the surface of a cell, and one gRNA that targets a gene that reduces or eliminates endogenous TCR expression.
Embodiment B130. The method of any one of embodiments B95-129, comprising one gRNA selected from a gRNA that targets TRAC, TRBC, B2M, HLA-A, or CIITA.
Embodiment B131. The method of any one of embodiments B95-B130, wherein the gRNA targets TRBC, wherein the gRNA comprises a guide sequence chosen from: i) SEQ ID NOs: 706-721; ii) at least 17, 18, 19, or 20 contiguous nucleotides of a sequence selected from SEQ ID NOs: 706-721; iii) a guide sequence at least 95%, 90%, or 85% identical to a sequence selected from SEQ ID NOs: 706-721; iv) a sequence that comprises 10 contiguous nucleotides±10 nucleotides of a genomic coordinate listed in Table 5A; v) at least 17, 18, 19, or 20 contiguous nucleotides of a sequence from (iv); or vi) a guide sequence that is at least 95%, 90%, or 85% identical to a sequence selected from (v).
Embodiment B132. The method of any one of embodiments B95-B130, wherein the gRNA targets TRBC, wherein the gRNA comprises a guide sequence chosen from: i) SEQ ID NOs: 618-669; ii) at least 17, 18, 19, or 20 contiguous nucleotides of a sequence selected from SEQ ID NOs: 618-669; iii) a guide sequence at least 95%, 90%, or 85% identical to a sequence selected from SEQ ID NOs: 618-669; iv) a sequence that comprises 10 contiguous nucleotides±10 nucleotides of a genomic coordinate listed in Table 5B; v) at least 17, 18, 19, or 20 contiguous nucleotides of a sequence from (iv); or vi) a guide sequence that is at least 95%, 90%, or 85% identical to a sequence selected from (v).
Embodiment B133. The method of any one of embodiments B95-B130, comprising at least two gRNAs selected from a gRNA that targets TRAC, TRBC, or B2M, wherein the two guide RNAs do not target the same gene.
Embodiment B134. The method of any one of embodiments B95-B130, comprising at least two gRNAs selected from a gRNA that targets TRAC, TRBC, or HLA-A, wherein the two guide RNAs do not target the same gene.
Embodiment B135. The method of any one of embodiments B95-B130, comprising one guide RNA that targets TRAC, and one gRNA that targets TRBC.
Embodiment B136. The method of any one of embodiments B95-B130, comprising one guide RNA that targets B2M, and one gRNA that targets CIITA.
Embodiment B137. The method of any one of embodiments B95-B130, comprising one guide RNA that targets HLA-A, and one gRNA that targets CIITA, optionally wherein the cell is homozygous for HLA-B and homozygous for HLA-C.
Embodiment B138. The method of any one of embodiments B95-B130, comprising one guide RNA that targets TRAC, and one gRNA that targets TRBC, and one gRNA that targets B2M.
Embodiment B139. The method of any one of embodiments B95-B130, comprising one guide RNA that targets TRAC, and one gRNA that targets TRBC, and one gRNA that targets HLA-A, optionally wherein the cell is homozygous for HLA-B and homozygous for HLA-C.
Embodiment B140. The method of any one of embodiments B95-B130, comprising one guide RNA that targets TRAC, and one gRNA that targets TRBC, one gRNA that targets B2M, and one gRNA that targets CIITA.
Embodiment B1141. The method of any one of embodiments B95-B130, comprising one guide RNA that targets TRAC, and one gRNA that targets TRBC, one gRNA that targets HLA-A, and one gRNA that targets CIITA, optionally wherein the cell is homozygous for HLA-B and homozygous for HLA-C.
Embodiment B142. The method of any one of the preceding embodiments, wherein the method generates a cytosine (C) to thymine (T) conversion within a target sequence.
Embodiment B143. The method of any one of the preceding embodiments, wherein the method causes at least 60% C-to-T conversion relative to the total edits in the target sequence.
Embodiment B144. The method of any one of the preceding embodiments, wherein the method causes at least 65%, 70%, 75%, 80%, 85%, 90%, 95%, 98%, or 99% C-to-T conversion relative to the total edits in the target sequence.
Embodiment B145. The method of any one of the preceding embodiments, wherein the ratio of C-to-T conversion to unintended edits is larger than 1:1.
Embodiment B146. The method of any one of the preceding embodiments, wherein the ratio of C-to-T conversion to unintended edits is from 2:1 to 99:1.
Embodiment B147. The method of any one of the preceding embodiments, wherein the ratio of C-to-T conversion to unintended edits is 2:1, 3:1, 4:1, 5:1, 6:1, 7:1, or 8:1.
Embodiment B148. The method of any one of the preceding embodiments, wherein the method causes the cytidine deaminase to make a base edit corresponding to any one of positions −1 to 10 relative to the 5′ end of the guide sequence.
Embodiment B149. The method of any one of the preceding embodiments, wherein the method causes the cytidine deaminase to make a base edit at a position 1, 2, 3, 4, 5, 6, 7, 8, 9, or 10 nucleotides from the 5′ end of the guide sequence.
Embodiment B150. The method of any one of the preceding embodiments, wherein the first mRNA, the second mRNA, and the guide RNA if present, delivered at a ratio of about 6:2:3 (w:w:w).
Embodiment B151. The method of any one of the preceding embodiments, wherein the mRNA, composition, or LNP is administered at a total RNA amount from 5 to 600 ng.
Embodiment B152. The method of any one of the preceding embodiments, wherein the total RNA amount is from 8 to 550 ng.
Embodiment B153. The method of any one of the preceding embodiments, wherein the total RNA amount is from 35 to 550 ng.
Embodiment B1154. The method of any one of the preceding embodiments, wherein the total RNA amount is from 70 to 550 ng.
Embodiment B155. The method of any one of the preceding embodiments, wherein the total RNA amount is from 138 to 550 ng.
Embodiment B156. The method of any one of the preceding embodiments, wherein the total RNA amount is from 275 to 550 ng.
Embodiment B157. The method, cell, or engineered cell of any one of the preceding embodiments, wherein the cell is a lymphocyte.
Embodiment B158. The method, cell, or engineered cell of any one of the preceding embodiments, wherein the cell is a B lymphocyte.
Embodiment B159. The method, cell, or engineered cell of any one of the preceding embodiments, wherein the cell is a T lymphocyte.
Embodiment B160. The method or use of any one of the preceding embodiments, wherein the modification of the target gene is in vivo.
Embodiment B161. The method or use of any one of the preceding embodiments, wherein the modification of the target gene is ex vivo.
Embodiment B162. The method or use of any one of the preceding embodiments, wherein the modification of the target gene reduces or eliminates expression of the target gene.
Embodiment B163. The method or use of any one of the preceding embodiments, wherein the genome editing or modification of the target gene reduces expression of the target gene by at least 50%, 55%, 60%, 65%, 70%, 75%, 80%, 85%, 90%, or 95%.
Embodiment B164. The method or use of any one of the preceding embodiments, wherein the genome editing or modification of the target gene produces a missense mutation in the gene.
Embodiment B165. A polypeptide comprising a cytidine deaminase and an RNA-guided nickase, wherein the polypeptide does not comprise a uracil glycosylase inhibitor (UGI).
Embodiment B166. A ribonucleoprotein complex (RNP) comprising the polypeptide of embodiment B165 and a guide RNA, wherein if the RNP comprises a SpyCas9 nickase, then the guide RNA is a Spy guide RNA, and wherein if the RNP comprises a NmeCas nickase, then the guide RNA is a Nme guide RNA.
Embodiment B167. A composition comprising a first polypeptide comprising a cytidine deaminase and an RNA-guided nickase, wherein the first polypeptide does not comprise a uracil glycosylase inhibitor (UGI), and a second polypeptide comprising a UGI, wherein the second polypeptide is different from the first polypeptide.
Embodiment B168. The polypeptide, RNP or composition of any one of embodiments B165-B167, wherein the cytidine deaminase is fused to the RNA-guided nickase via a peptide linker, optionally XTEN.
Embodiment B169. The polypeptide, RNP or composition of any one of embodiments B165-B167, wherein the cytidine deaminase is attached to a linker comprising an organic molecule, polymer, or chemical moiety.
Embodiment B170. A pharmaceutical composition comprising the mRNA, RNP, composition, or polypeptide of any of the preceding embodiments and a pharmaceutically acceptable carrier.
Embodiment B171. A kit comprising the mRNA, RNP, composition, or polypeptide of any of the preceding embodiments.
Embodiment B172. The mRNA, RNP, composition, method, cell, or engineered cell of any one of the preceding embodiments, wherein the polypeptide comprising a cytidine deaminase and an RNA-guided nickase includes: the cytidine deaminase, a linker, and the RNA-guided nickase in amino to carboxy terminal order.
Embodiment B173. A method of altering a DNA sequence within a TRAC gene, comprising delivering to a cell:
a. a gRNA comprising a guide sequence chosen from: i) SEQ ID NOs: 706-721; ii) at least 17, 18, 19, or 20 contiguous nucleotides of a sequence selected from SEQ ID NOs: 706-721; iii) a guide sequence at least 95%, 90%, or 85% identical to a sequence selected from SEQ ID NOs: 706-721; iv) a sequence that comprises 10 contiguous nucleotides±10 nucleotides of a genomic coordinate listed in Table 5A; v) at least 17, 18, 19, or 20 contiguous nucleotides of a sequence from (iv); or vi) a guide sequence that is at least 95%, 90%, or 85% identical to a sequence selected from (v); or
b. a nucleic acid encoding a gRNA of (a.).
Embodiment B174. A method of reducing the expression of a TRAC gene, comprising delivering to a cell:
a. a gRNA comprising a guide sequence chosen from: i) SEQ ID NOs: 706-721; ii) at least 17, 18, 19, or 20 contiguous nucleotides of a sequence selected from SEQ ID NOs: 706-721; iii) a guide sequence at least 95%, 90%, or 85% identical to a sequence selected from SEQ ID NOs: 706-721; iv) a sequence that comprises 10 contiguous nucleotides±10 nucleotides of a genomic coordinate listed in Table 5A; v) at least 17, 18, 19, or 20 contiguous nucleotides of a sequence from (iv); or vi) a guide sequence that is at least 95%, 90%, or 85% identical to a sequence selected from (v); or
b. a nucleic acid encoding a gRNA of (a.).
Embodiment B175. A method of immunotherapy comprising administering a composition comprising an engineered cell to a subject,
wherein the cell comprises a genomic modification of at least one nucleotide within the genomic coordinates selected from:
chr14: 22547596-22547616; chr14: 22550570-22550590; chr14: 22547763-22547783; chr14: 22550596-22550616; chr14: 22550566-22550586; chr14: 22547753-22547773; chr14: 22550601-22550621; chr14: 22550599-22550619; chr14: 22547583-22547603; chr14: 22547671-22547691; chr14: 22547770-22547790; chr14: 22547676-22547696; chr14: 22547772-22547792; chr14: 22547771-22547791; chr14: 22547733-22547753; chr14: 22547776-22547796; or
wherein the cell is engineered by delivering to the cell:
a. a gRNA comprising a guide sequence chosen from: i) SEQ ID NOs: 706-721; ii) at least 17, 18, 19, or 20 contiguous nucleotides of a sequence selected from SEQ ID NOs: 706-721; iii) a guide sequence at least 95%, 90%, or 85% identical to a sequence selected from SEQ ID NOs: 706-721; iv) a sequence that comprises 10 contiguous nucleotides±10 nucleotides of a genomic coordinate listed in Table 5A; v) at least 17, 18, 19, or 20 contiguous nucleotides of a sequence from (iv); or vi) a guide sequence that is at least 95%, 90%, or 85% identical to a sequence selected from (v); or
b. a nucleic acid encoding a gRNA of (a.).
Embodiment B3176. The method of any one of embodiments B173-B175, wherein the guide sequence comprises any one of SEQ ID NOs: 706-709.
Embodiment B177. The method of any one of embodiments B173-B175, wherein the guide sequence comprises any one of SEQ ID NOs: 706-708.
Embodiment B178. The method of any one of embodiments B173-B175, wherein the guide sequence comprises SEQ ID NO: 706.
Embodiment B179. The method of any one of embodiments B173-B175, wherein the guide sequence comprises SEQ ID NO: 707.
Embodiment B180. The method of any one of embodiments B173-B175, wherein the guide sequence comprises SEQ ID NO: 708.
Embodiment B181. A method of altering a DNA sequence within a TRBC1 and/or TRBC2 gene, comprising delivering a composition to a cell, wherein the composition comprises:
a. a gRNA comprising a guide sequence chosen from: i) SEQ ID NOs: 618-669; ii) at least 17, 18, 19, or 20 contiguous nucleotides of a sequence selected from SEQ ID NOs: 618-669; iii) a guide sequence at least 95%, 90%, or 85% identical to a sequence selected from SEQ ID NOs: 618-669; iv) a sequence that comprises 10 contiguous nucleotides±10 nucleotides of a genomic coordinate listed in Table 5B; v) at least 17, 18, 19, or 20 contiguous nucleotides of a sequence from (iv); or vi) a guide sequence that is at least 95%, 90%, or 85% identical to a sequence selected from (v); or
b. a nucleic acid encoding a guide RNA of (a.).
Embodiment B182. A method of reducing the expression of a TRBC1 and/or TRBC2 gene, comprising delivering a composition to a cell, wherein the composition comprises:
a. a gRNA comprising a guide sequence chosen from: i) SEQ ID NOs: 618-669; ii) at least 17, 18, 19, or 20 contiguous nucleotides of a sequence selected from SEQ ID NOs: 618-669; iii) a guide sequence at least 95%, 90%, or 85% identical to a sequence selected from SEQ ID NOs: 618-669; iv) a sequence that comprises 10 contiguous nucleotides±10 nucleotides of a genomic coordinate listed in Table 5B; v) at least 17, 18, 19, or 20 contiguous nucleotides of a sequence from (iv); or vi) a guide sequence that is at least 95%, 90%, or 85% identical to a sequence selected from (v); or
b. a nucleic acid encoding a guide RNA of (a.).
Embodiment B183. A method of immunotherapy comprising administering a composition comprising an engineered cell to a subject,
wherein the cell comprises a modification of at least one nucleotide within the genomic coordinates selected from: chr7: 142791757-142791777; chr7: 142801104-142801124; chr7: 142791811-142791831; chr7: 142801158-142801178; chr7: 142792728-142792748; chr7: 142791719-142791739; chr7: 142791766-142791786; chr7: 142801113-142801133; chr7: 142791928-142791948; chr7: 142801275-142801295; chr7: 142792062-142792082; chr7: 142801409-142801429; chr7: 142792713-142792733; chr7: 142802126-142802146; chr7: 142791808-142791828; chr7: 142801155-142801175; chr7: 142792003-142792023; chr7: 142801350-142801370; chr7: 142791760-142791780; chr7: 142791715-142791735; chr7: 142792781-142792801; chr7: 142792040-142792060; chr7: 142801387-142801407; chr7: 142791862-142791882; chr7: 142791716-142791736; chr7: 142791787-142791807; chr7: 142791759-142791779; chr7: 142801106-142801126; chr7: 142791807-142791827; chr7: 142801154-142801174; chr7: 142791879-142791899; chr7: 142801226-142801246; chr7: 142791805-142791825; chr7: 142791700-142791720; chr7: 142791765-142791785; chr7: 142801112-142801132; chr7: 142791820-142791840; chr7: 142791872-142791892; chr7: 142801219-142801239; chr7: 142791700-142791720; chr7: 142791806-142791826; chr7: 142801153-142801173; chr7: 142792035-142792055; chr7: 142792724-142792744; chr7: 142792754-142792774; chr7: 142791804-142791824; chr7: 142792684-142792704; chr7: 142791823-142791843; chr7: 142792728-142792748; chr7: 142792721-142792741; chr7: 142792749-142792769; chr7: 142792685-142792705; chr7: 142791816-142791836; chr7: 142801163-142801183; chr7: 142792686-142792706; chr7: 142791793-142791813; chr7: 142793110-142793130; chr7: 142791815-142791835; chr7: 142801162-142801182; chr7: 142792770-142792790; chr7: 142792047-142792067; chr7: 142801394-142801414; chr7: 142791871-142791891; chr7: 142801218-142801238; chr7: 142791894-142791914; chr7: 142792723-142792743; chr7: 142792724-142792744; chr7: 142791897-142791917; chr7: 142801244-142801264; chr7: 142792757-142792777; chr7: 142792740-142792760; chr7: 142792758-142792778; or
wherein the cell is engineered by delivering to a cell:
a. a gRNA comprising a guide sequence chosen from: i) SEQ ID NOs: 618-669; ii) at least 17, 18, 19, or 20 contiguous nucleotides of a sequence selected from SEQ ID NOs: 618-669; iii) a guide sequence at least 95%, 90%, or 85% identical to a sequence selected from SEQ ID NOs: 618-669; iv) a sequence that comprises 10 contiguous nucleotides±10 nucleotides of a genomic coordinate listed in Table 5B; v) at least 17, 18, 19, or 20 contiguous nucleotides of a sequence from (iv); or vi) a guide sequence that is at least 95%, 90%, or 85% identical to a sequence selected from (v); or
b. a nucleic acid encoding a guide RNA of (a.).
Embodiment B184. The method of any one of embodiments B181-B183, wherein the guide sequence comprises any one of SEQ ID NOs: 618-627.
Embodiment B3185. The method of any one of embodiments B181-B184, wherein the guide sequence comprises any one of SEQ ID NOs: 618-621.
Embodiment B186. The method of any one of embodiments B181-B185, wherein the guide sequence comprises SEQ ID NO: 618.
Embodiment B187. The method of any one of embodiments B181-B185, wherein the guide sequence comprises SEQ ID NO: 619.
Embodiment B188. The method of any one of embodiments B181-B185, wherein the guide sequence comprises SEQ ID NO: 620.
Embodiment B189. The method of any one of embodiments B181-B185, wherein the guide sequence comprises SEQ ID NO: 621.
Embodiment B190. The method of any one of embodiments B173-B189, wherein the composition further comprises the mRNA or composition of any one of the preceding embodiments relating to mRNA or compositions.
Embodiment B191. A composition comprising:
a. a gRNA comprising a guide sequence chosen from: i) SEQ ID NOs: 706-721; ii) at least 17, 18, 19, or 20 contiguous nucleotides of a sequence selected from SEQ ID NOs: 706-721; iii) a guide sequence at least 95%, 90%, or 85% identical to a sequence selected from SEQ ID NOs: 706-721; iv) a sequence that comprises 10 contiguous nucleotides±10 nucleotides of a genomic coordinate listed in Table 5A; v) at least 17, 18, 19, or 20 contiguous nucleotides of a sequence from (iv); or vi) a guide sequence that is at least 95%, 90%, or 85% identical to a sequence selected from (v); and optionally
b. the mRNA or composition of any one of the preceding embodiments relating to mRNA or compositions.
Embodiment B192. The composition of embodiment B191, wherein the guide sequence comprises any one of SEQ ID NOs: 706-709.
Embodiment B193. The composition of embodiment B191 or B192, for use in altering a DNA sequence within the TRAC gene in a cell.
Embodiment B194. The composition of any one of embodiments B191-B193 for use in reducing the expression of the TRAC gene in a cell.
Embodiment B195. The composition of any one of embodiments B191-B194, for use in immunotherapy of a subject.
Embodiment B196. A composition comprising:
a. a gRNA comprising a guide sequence chosen from: i) SEQ ID NOs: 618-669; ii) at least 17, 18, 19, or 20 contiguous nucleotides of a sequence selected from SEQ ID NOs: 618-669; iii) a guide sequence at least 95%, 90%, or 85% identical to a sequence selected from SEQ ID NOs: 618-669; iv) a sequence that comprises 10 contiguous nucleotides±10 nucleotides of a genomic coordinate listed in Table 5B; v) at least 17, 18, 19, or 20 contiguous nucleotides of a sequence from (iv); or vi) a guide sequence that is at least 95%, 90%, or 85% identical to a sequence selected from (v); and optionally
b. the mRNA or composition of any one of the preceding embodiments relating to mRNA or compositions.
Embodiment B197. The composition of embodiment B196, wherein the guide sequence comprises any one of SEQ ID NOs: 618-621.
Embodiment B198. The composition of embodiment B196 or B197, for use in altering a DNA sequence within the TRBC1 and/or TRBC2 gene in a cell.
Embodiment B199. The composition of any one of embodiments B196-B198 for use in reducing the expression of the TRBC1 and/or TRBC2 gene in a cell.
Embodiment B200. The composition of any one of embodiments B196-B199, for use in immunotherapy of a subject.
Embodiment B201. A cell, altered by the method of any one of embodiments B120-B121, and 161-178.
Embodiment B202. The cell according to embodiment B201, wherein the cell is altered ex vivo.
Embodiment B203. The cell according to embodiment B201 or B202, wherein the cell is a T cell.
Embodiment B204. The cell according to any of embodiments B201-B203, wherein the cell is a CD4⁺ or CD8⁺ T cell.
Embodiment B205. The cell according to any of embodiments B201-B204, wherein the cell is a mammalian, primate, or human cell.
Embodiment B206. The cell according to any of embodiments B201-B205, for use in immunotherapy of a subject.
Embodiment B207. An engineered cell which has reduced or eliminated surface expression of TRAC, comprising a genetic modification in a human TRAC gene, wherein the genetic modification comprises a modification of at least one nucleotide within the genomic coordinates selected from:
chr14: 22547596-22547616; chr14: 22550570-22550590; chr14: 22547763-22547783; chr14: 22550596-22550616; chr14: 22550566-22550586; chr14: 22547753-22547773; chr14: 22550601-22550621; chr14: 22550599-22550619; chr14: 22547583-22547603; chr14: 22547671-22547691; chr14: 22547770-22547790; chr14: 22547676-22547696; chr14: 22547772-22547792; chr14: 22547771-22547791; chr14: 22547733-22547753; chr14: 22547776-22547796.
Embodiment B208. An engineered cell which has reduced or eliminated surface expression of TRBC1/2, comprising a genetic modification in a human TRBC1/2 gene, wherein the genetic modification comprises a modification of at least one nucleotide within the genomic coordinates selected from:
chr7: 142791757-142791777; chr7: 142801104-142801124; chr7: 142791811-142791831; chr7: 142801158-142801178; chr7: 142792728-142792748; chr7: 142791719-142791739; chr7: 142791766-142791786; chr7: 142801113-142801133; chr7: 142791928-142791948; chr7: 142801275-142801295; chr7: 142792062-142792082; chr7: 142801409-142801429; chr7: 142792713-142792733; chr7: 142802126-142802146; chr7: 142791808-142791828; chr7: 142801155-142801175; chr7: 142792003-142792023; chr7: 142801350-142801370; chr7: 142791760-142791780; chr7: 142791715-142791735; chr7: 142792781-142792801; chr7: 142792040-142792060; chr7: 142801387-142801407; chr7: 142791862-142791882; chr7: 142791716-142791736; chr7: 142791787-142791807; chr7: 142791759-142791779; chr7: 142801106-142801126; chr7: 142791807-142791827; chr7: 142801154-142801174; chr7: 142791879-142791899; chr7: 142801226-142801246; chr7: 142791805-142791825; chr7: 142791700-142791720; chr7: 142791765-142791785; chr7: 142801112-142801132; chr7: 142791820-142791840; chr7: 142791872-142791892; chr7: 142801219-142801239; chr7: 142791700-142791720; chr7: 142791806-142791826; chr7: 142801153-142801173; chr7: 142792035-142792055; chr7: 142792724-142792744; chr7: 142792754-142792774; chr7: 142791804-142791824; chr7: 142792684-142792704; chr7: 142791823-142791843; chr7: 142792728-142792748; chr7: 142792721-142792741; chr7: 142792749-142792769; chr7: 142792685-142792705; chr7: 142791816-142791836; chr7: 142801163-142801183; chr7: 142792686-142792706; chr7: 142791793-142791813; chr7: 142793110-142793130; chr7: 142791815-142791835; chr7: 142801162-142801182; chr7: 142792770-142792790; chr7: 142792047-142792067; chr7: 142801394-142801414; chr7: 142791871-142791891; chr7: 142801218-142801238; chr7: 142791894-142791914; chr7: 142792723-142792743; chr7: 142792724-142792744; chr7: 142791897-142791917; chr7: 142801244-142801264; chr7: 142792757-142792777; chr7: 142792740-142792760; chr7: 142792758-142792778.
Embodiment B209. One or more lipid nucleic acid assembly compositions, optionally lipid nanoparticles, comprising:
(a) a first mRNA comprising a first open reading frame encoding a polypeptide comprising a cytidine deaminase and an RNA-guided nickase;
(b) a second mRNA comprising a second open reading frame encoding a uracil glycosylase inhibitor (UGI); and
(c) one or more guide RNAs.
Embodiment B210. The method, cell, or engineered cell of any one of the preceding embodiments, wherein the cell is an immune cell.
Embodiment B211. The method, cell, or engineered cell of any one of the preceding embodiments, wherein the cell is a lymphocyte.
Embodiment B212. The method, cell, or engineered cell of any one of the preceding embodiments, wherein the cell is a T cell.

LENGTHY TABLES
The patent application contains a lengthy table section. A copy of the table is available in electronic form from the USPTO web site (https://seqdata.uspto.gov/?pageRequest=docDetail&DocID=US20240002820A1). An electronic copy of the table will also be available from the USPTO upon request and payment of the fee set forth in 37 CFR 1.19(b)(3).

We claim:

1. A composition comprising a first mRNA comprising a first open reading frame encoding a polypeptide comprising a cytidine deaminase and an RNA-guided nickase, and a second mRNA comprising a second open reading frame encoding a uracil glycosylase inhibitor (UGI), wherein the second mRNA is different from the first mRNA, optionally wherein the composition comprises lipid nanoparticles.

2. The composition of claim 1, wherein the first open reading frame does not comprise a sequence encoding a UGI.

3. The composition of claim 1 or 2, wherein the composition comprises a first composition and a second composition, wherein the first composition comprises a first mRNA comprising a first open reading frame encoding a polypeptide comprising a cytidine deaminase and an RNA-guided nickase and does not comprise a uracil glycosylase inhibitor (UGI), and the second composition comprises a second mRNA comprising a second open reading frame encoding a uracil glycosylase inhibitor (UGI), wherein the second mRNA is different from the first mRNA, optionally wherein the compositions comprise lipid nanoparticles.

4. The composition of any one of claims 1-3, wherein the first mRNA and the second mRNAs are in the same or separate vials.

5. A method of modifying a target gene comprising delivering to a cell a first mRNA comprising a first open reading frame encoding a first polypeptide comprising a cytidine deaminase and an RNA-guided nickase, a second mRNA comprising a second open reading frame encoding a uracil glycosylase inhibitor (UGI), wherein the second mRNA is different from the first mRNA, and at least one guide RNA (gRNA).

6. The method of claim 5, wherein if the nickase is a SpyCas9 nickase, then the gRNA is a SpyCas9 gRNA, and if the nickase is a NmeCas9 nickase, then the gRNA is a Nme gRNA.

7. The method of claim 5 or 6, wherein the first open reading frame does not comprise a sequence encoding a UGI.

8. The composition or method of any one of claims 1-7, wherein the molar ratio of the second mRNA to the first mRNA is from 1:1 to 30:1.

9. The composition or method of any one of claims 1-7, wherein the molar ratio is from 2:1 to 30:1.

10. The composition or method of any one of claims 1-7, wherein the molar ratio is from 7:1 to 22:1.

11. An mRNA comprising an open reading frame encoding a polypeptide comprising a cytidine deaminase and an RNA-guided nickase, wherein the polypeptide does not comprise a uracil glycosylase inhibitor (UGI).

12. A method of modifying at least one cytidine within a target gene in a cell, comprising expressing in the cell or contacting the cell with: (i) a first polypeptide comprising a cytidine deaminase and an RNA-guided nickase, wherein the first polypeptide does not comprise a uracil glycosylase inhibitor (UGI); (ii) a UGI polypeptide; and (iii) at least one guide RNA (gRNA) wherein the first polypeptide and gRNA form a complex with the target gene and modify the at least one cytidine in the target gene.

13. The method of claim 12, wherein if the nickase is a SpyCas9 nickase, then the gRNA is a SpyCas9 gRNA, and if the nickase is a NmeCas9 nickase, then the gRNA is a Nme gRNA.

14. The method of claim 12 or 13, wherein the ratio of the UGI polypeptide to the first polypeptide is from 10:1 to 50:1.

15. A cell, wherein the mRNA or composition of any one of claims 1-4 and 8-11 has been introduced to the cell, wherein the cell has been modified after the introduction.

16. An engineered cell altered by the method of claims 5-10 and 12-14.

17. An engineered cell comprising at least one base edit and/or indel, wherein the base edit and/or indel is made by contacting a cell with a composition comprising a first mRNA comprising a first open reading frame encoding a polypeptide comprising a cytidine deaminase and an RNA-guided nickase, and a second mRNA comprising a second open reading frame encoding a uracil glycosylase inhibitor (UGI), wherein the second mRNA is different from the first mRNA.

18. The engineered cell of claim 17, wherein the first open reading frame does not comprise a sequence encoding a UGI.

19. The mRNA, composition, method, cell, or engineered cell of any one of claims 1-18, wherein the cytidine deaminase is
(i) an enzyme of APOBEC family, optionally an enzyme of APOBEC3 subgroup;
(ii) a cytidine deaminase comprising an amino acid sequence that is at least 80% identical to any one of SEQ ID NOs: 40, 41, and 960-1023;
(iii) a cytidine deaminase comprising an amino acid sequence that is at least 80% identical to any one of SEQ ID NOs: 40, 41, and 960-1013;
(iv) a cytidine deaminase comprising an amino acid sequence that is at least 80% identical to any one of SEQ ID NOs: 40, 41, 976, 977, 979, 980, 984-987, 993-1006, and 1009; or
(v) a cytidine deaminase comprising an amino acid sequence that is at least 80% identical to any one of SEQ ID NOs: 40, 976, 981, 984, 986, and 1014-1023.

20. The mRNA, composition, method, cell, or engineered cell of claim 19, wherein the cytidine deaminase comprises an amino acid sequence with at least 80%, 85%, 87%, 90%, 95%, 98%, 99%, or 100% identity to SEQ ID NO: 40, 41, and 960-1023.

21. The mRNA, composition, method, cell, or engineered cell of claim 19, wherein the cytidine deaminase comprises an amino acid sequence with at least 80%, 85%, 87%, 90%, 95%, 98%, 99%, or 100% identity to SEQ ID NO: 40, 41, and 960-1013.

22. The mRNA, composition, method, cell, or engineered cell of claim 19, wherein the cytidine deaminase comprises an amino acid sequence with at least 80%, 85%, 87%, 90%, 95%, 98%, 99%, or 100% identity to SEQ ID NO: 40, 41, 976, 977, 979, 980, 984-987, 993-1006, and 1009.

23. The mRNA, composition, method, cell, or engineered cell of claim 19, wherein the cytidine deaminase comprises an amino acid sequence with at least 80%, 85%, 87%, 90%, 95%, 98%, 99%, or 100% identity to SEQ ID NO: 40, 976, 981, 984, 986, 1014-1023.

24. The mRNA, composition, method, cell, or engineered cell of any one of claims 1-23, wherein the cytidine deaminase is an APOBEC3A deaminase (A3A).

25. The mRNA, composition, method, cell, or engineered cell of claim 24, wherein the A3A comprises an amino acid sequence of SEQ ID NO: 40 or an amino acid sequence with at least 87%, at least 90%, at least 95%, at least 98%, at least 99% identity to SEQ ID NO: 40.

26. The mRNA, composition, method, cell, or engineered cell of any one of claims 24-25, wherein the A3A is a human A3A.

27. The mRNA, composition, method, cell, or engineered cell of any one of claims 24-26, wherein the A3A is a wild-type A3A.

28. The mRNA, composition, method, cell, or engineered cell of claim 24, wherein the A3A comprises an amino acid sequence with at least 87%, 90%, 95%, 98%, 99%, or 100% identity to SEQ ID NO: 976, 977, 993-1006, and 1009.

29. The composition, method, cell, or engineered cell of any one of claims 1-28, wherein the UGI comprises an amino acid sequence of SEQ ID NO: 27 or an amino acid sequence with at least 80%, at least 90%, at least 95%, at least 98%, at least 99% identity to SEQ ID NO: 27.

30. The composition, method, or cell of any one of claims 1-4, 8-10, and 15-29, further comprising at least one guide RNA (gRNA).

31. The composition, method, cell, or engineered cell of any one of claims 1-4, 8-10, and 15-30, comprising a gRNA, wherein the gRNA is an sgRNA.

32. The composition, method, cell, or engineered cell of any one of claims 1-4, 8-10, and 15-31, comprising a gRNA, wherein the gRNA is a short-single guide RNA (short-sgRNA) comprising a conserved portion of an sgRNA comprising a hairpin region, wherein the hairpin region lacks at least 5-10 nucleotides and wherein the short-sgRNA comprises a 5′ end modification or a 3′ end modification or both.

33. The mRNA, composition, method, cell, or engineered cell of any one of claims 1-32, wherein the RNA-guided nickase is a Cas9 nickase.

34. The mRNA, composition, method, cell, or engineered cell of any one of claims 1-33, wherein the RNA-guided nickase is an S. pyogenes (Spy) Cas9 nickase.

35. The mRNA, composition, method, cell, or engineered cell of claim 34, wherein the RNA-guided nickase is a D10A SpyCas9 nickase.

36. The mRNA, composition, method, cell, or engineered cell of any one of claims 1-35, wherein the RNA-guided nickase comprises an amino acid sequence of any one of SEQ ID NOs: 70, 73, or 76 or an amino acid sequence having at least 80%, 90%, 95%, 98%, or 99% identity to any one of SEQ ID NOs: 70, 73, or 76.

37. The mRNA, composition, method, cell, or engineered cell of any one of claims 1-36, wherein the sequence encoding the RNA-guided nickase comprises a nucleotide sequence of any one of SEQ ID NOs: 72, 75, or 78 or a nucleotide sequence having at least 80%, 90%, 95%, 98%, or 99% identity to the nucleotide sequence of any one of SEQ ID NOs: 72, 75, or 78.

38. The mRNA, composition, method, cell, or engineered cell of any one of claims 1-37, wherein the sequence encoding the RNA-guided nickase comprises the nucleotide sequence of any one of SEQ ID NOs: 71, 72, 74, 75, or 77-90.

39. The mRNA, composition, method, cell, or engineered cell of any one of claims 1-33, wherein the RNA-guided nickase is a N. meningitidis (Nme) Cas9 nickase.

40. The mRNA, composition, method, cell, or engineered cell of claim 39, wherein the RNA-guided nickase is a D16A NmeCas9 nickase, optionally a D16A Nme2Cas9.

41. The mRNA, composition, method, cell, or engineered cell of any one of claims 1-33, 39, or 40, wherein the sequence encoding the RNA-guided nickase comprises the nucleotide sequence of any one of SEQ ID NOs: 380 and 387.

42. The mRNA, composition, method, cell, or engineered cell of any one of claims 1-41, wherein the mRNA comprises a 5′ UTR with at least 90% identity to any one of SEQ ID NOs: 91-98.

43. The mRNA, composition, method, cell, or engineered cell of any one of claims 1-42, wherein the mRNA comprises a 3′ UTR with at least 90% identity to any one of SEQ ID NOs: 99-106.

44. The mRNA, composition, method, cell, or engineered cell of any one of claims 1-43, wherein the mRNA further comprises a 5′ cap selected from Cap0, Cap1, Cap2, and a cap added co-transcriptionally or post-transcriptionally, optionally wherein the co-transcriptionally added cap is selected from anti-reverse cap analog (ARCA), AG (m7G(5′)ppp(5′)(2′OMeA)pG, or GG (m7G(5′)ppp(5′)(2′OMeG)pG, a cap added post-transcriptionally.

45. The mRNA, composition, method, cell, or engineered cell of any one of claims 1-44, wherein the mRNA further comprises a poly-adenylated (poly-A) tail, optionally wherein the poly-A tail is added to the mRNA by PCR tailing or enzymatic tailing and optionally wherein the poly-A tail comprises a sequence of SEQ ID NO: 109.

46. The mRNA, composition, method, cell, or engineered cell of any one of claims 1-45, wherein the open reading frame encoding a polypeptide comprising a cytidine deaminase and an RNA-guided nickase, and/or the open reading frame encoding a uracil glycosylase inhibitor (UGI) comprise (i) minimal adenine codons and/or minimal uridine codons; (ii) minimal adenine codons; (iii) codons that increase translation of the mRNA in a mammal; or (iv) codons that increase translation of the mRNA in a mammal, wherein the mammal is a human.

47. The mRNA, composition, method, cell, or engineered cell of any one of claims 1-46, wherein the cytidine deaminase is located N-terminal to the RNA-guided nickase in the polypeptide.

48. The mRNA, composition, method, cell, or engineered cell of any one of claims 1-47, wherein the encoded RNA-guided nickase comprises a nuclear localization signal (NLS).

49. The mRNA, composition, method, cell, or engineered cell of any one of claims 1-48, wherein the encoded RNA-guided nickase comprises a nuclear localization signal (NLS), and wherein the NLS is at the C-terminus of the RNA-guided nickase.

50. The mRNA, composition, method, cell, or engineered cell of any one of claims 1-49, wherein the encoded RNA-guided nickase comprises a nuclear localization signal (NLS), and wherein the NLS is at the N-terminus of the RNA-guided nickase, or wherein an NLS is fused to both the N-terminus and C-terminus of the RNA-guided nickase.

51. The mRNA, composition, method, cell, or engineered cell of any one of claims 1-50, wherein the encoded RNA-guided nickase comprises a nuclear localization signal (NLS), and wherein a linker is present between the N-terminus of the RNA-guided nickase and the NLS, optionally wherein the linker is a peptide linker.

52. The mRNA, composition, method, cell, or engineered cell of any one of claims 1-51, wherein the encoded RNA-guided nickase comprises a nuclear localization signal (NLS), and wherein the NLS comprises a sequence having at least 80%, 85%, 90%, or 95% identity to any one of SEQ ID NOs: 63 and 110-122.

53. The mRNA, composition, method, cell, or engineered cell of any one of claims 1-52, wherein the encoded RNA-guided nickase comprises a nuclear localization signal (NLS), and wherein the NLS comprises the sequence of any one of SEQ ID NOs: 63 and 110-122.

54. The mRNA, composition, method, cell, or engineered cell of any one of claims 1-53, wherein the encoded RNA-guided nickase comprises a nuclear localization signal (NLS), and wherein the NLS is encoded by a sequence having at least 80%, 85%, 90%, 95%, 98% or 100% identity to the sequence of any one of SEQ ID NOs: 123-135.

55. The mRNA, composition, method, cell, or engineered cell of any one of claims 1-54, wherein the encoded RNA-guided nickase comprises a nuclear localization signal (NLS), and wherein the cytidine deaminase is located N-terminal to the NLS in the polypeptide.

56. The mRNA, composition, method, cell, or engineered cell of any one of claims 1-55, wherein the encoded RNA-guided nickase comprises a nuclear localization signal (NLS), and wherein the RNA-guided nickase is located N-terminal to the NLS in the polypeptide.

57. The mRNA, composition, method, cell, or engineered cell of any one of claims 1-56, wherein the open reading frame encoding a polypeptide comprising a cytidine deaminase and an RNA-guided nickase comprises a sequence having at least 80%, 85%, 90%, 95%, 98% or 100% identity to the sequence of SEQ ID NO:1.

58. The mRNA, composition, method, cell, or engineered cell of any one of claims 1-57, wherein the open reading frame encoding a polypeptide comprising a cytidine deaminase and an RNA-guided nickase comprises a sequence having at least 80%, 85%, 90%, 95%, 98% or 100% identity to the sequence of SEQ ID NO: 4.

59. The mRNA, composition, method, cell, or engineered cell of any one of claims 1-58, wherein the open reading frame encoding a polypeptide comprising a cytidine deaminase and an RNA-guided nickase comprises a sequence having at least 80%, 85%, 90%, 95%, 98%, 99% or 100% identity to the sequence of SEQ ID NO: 321.

60. The mRNA, composition, method, cell, or engineered cell of any one of claims 1-59, wherein the open reading frame encoding a polypeptide comprising a cytidine deaminase and an RNA-guided nickase comprises a sequence having at least 80%, 85%, 90%, 95%, 98%, 99% or 100% identity to the sequence of SEQ ID NO: 313.

61. The mRNA, composition, method, cell, or engineered cell of any one of claims 1-60, wherein at least 10% of the uridine in the mRNA is substituted with a modified uridine.

62. The mRNA, composition, method, cell or engineered cell of claim 61, wherein the modified uridine is one or more of N1-methyl-pseudouridine, pseudouridine, 5-methoxyuridine, or 5-iodouridine.

63. The mRNA, composition, method, cell, or engineered cell of any one of claims 61-62, wherein 15% to 45% of the uridine is substituted with the modified uridine.

64. The mRNA, composition, method, cell, or engineered cell of any one of claims 61-63, wherein at least 20% or at least 30%, at least 80% or at least 90%, or 100% of the uridine is substituted with the modified uridine.

65. The mRNA, composition, method, or cell of any one of the preceding claims 61-64, further encoding a peptide linker between the cytidine deaminase and RNA-guided nickase, optionally wherein the peptide linker is XTEN or the peptide linker comprises a sequence of GTKDSTKDIPETPSKD (SEQ ID NO: 268).

66. The mRNA, composition, method, or cell of any one of claims 61-65, further encoding a peptide linker between the cytidine deaminase and RNA-guided nickase, wherein the peptide linker comprises at least 1, at least 2, at least 3, at least 4, at least 5, at least 6, at least 7, at least 8, at least 9, at least 10, at least 15, at least 20, at least 25, at least 30, at least 40, at least 50, or more amino acids.

67. The mRNA, composition, method, or cell of any one of claims 61-66, further encoding a peptide linker between the cytidine deaminase and RNA-guided nickase, wherein the peptide linker comprises one or more sequences selected from SEQ ID NOs: 46-59, 61 and 211-272.

68. A polypeptide encoded by any one of the mRNAs of any one of claims 1-28 and 33-67.

69. A ribonucleoprotein complex (RNP) comprising (i) a polypeptide encoded by any one of the mRNAs of any one of claims 1-28 and 33-67; and (ii) a guide RNA.

70. A vector comprising any one of the mRNAs of any one of claims 1-28 and 33-67.

71. An expression construct comprising a promoter operably linked to a sequence encoding any one of the mRNAs of any one of claims 1-28, 33-60, and 65-67.

72. A plasmid comprising the expression construct of claim 71.

73. A host cell comprising the vector of claim 70, the expression construct of claim 71, or the plasmid of claim 72.

74. The mRNA or composition of any one of claims 1-4, 8-11, and 19-67, wherein the mRNA or composition is formulated as a lipid nucleic acid assembly composition, optionally a lipid nanoparticle.

75. Use of the mRNA or composition according to any one of claims 1-4, 8-11, and 19-67 for modifying a target gene in a cell.

76. Use of the mRNA or composition according to any one of claims 1-4, 8-11, and 19-67 for the manufacture of a medicament for modifying a target gene in a cell.

77. A method of modifying a target gene in a cell, comprising delivering to the cell one or more lipid nucleic acid assembly compositions, optionally lipid nanoparticles, comprising:
(a) a first mRNA comprising a first open reading frame encoding a polypeptide comprising a cytidine deaminase and an RNA-guided nickase;
(b) a second mRNA comprising a second open reading frame encoding a uracil glycosylase inhibitor (UGI); and
(c) one or more guide RNAs.

78. The method of claim 77, wherein parts (a), (b), and (c) are each in separate lipid nucleic acid assembly compositions.

79. The method of claim 77, wherein parts (a), (b), and (c) are in the same lipid nucleic acid assembly composition.

80. The method of any one of claims 77-79, wherein the one or more guide RNAs are each in separate lipid nucleic acid assembly compositions.

81. The method of any one of claims 77-80, comprising delivering to the cell a lipid nucleic acid assembly composition comprising a first mRNA comprising a first open reading frame encoding a polypeptide comprising a cytidine deaminase and an RNA-guided nickase and a second mRNA comprising a second open reading frame encoding a uracil glycosylase inhibitor (UGI) in the same lipid nucleic acid assembly composition.

82. The method of any one of claims 77-81, comprising delivering to the cell a first lipid nucleic acid assembly composition comprising a first mRNA comprising a first open reading frame encoding a polypeptide comprising a cytidine deaminase and an RNA-guided nickase, and a second lipid nucleic acid assembly composition comprising a second mRNA comprising a second open reading frame encoding a uracil glycosylase inhibitor (UGI).

83. The method of any one of claims 77-82, further comprising delivering one or more guide RNAs in one or more lipid nucleic acid assembly compositions that are separate from the lipid nucleic acid assembly compositions comprising the cytidine deaminase and UGI.

84. The method of claims any one of claims 77-83, wherein at least 2, 3, 4, 5, 6, 7, 8, 9, or 10 lipid nucleic acid assembly compositions are delivered to the cell.

85. The method of any one of claims 77-84, wherein at least one lipid nucleic acid assembly composition comprises lipid nanoparticle (LNPs), optionally wherein all lipid nucleic acid assembly compositions comprise LNPs.

86. The method of any one of claims 77-85, wherein at least one lipid nucleic acid assembly composition is a lipoplex composition.

87. The method of any one of claims 77-86, wherein the lipid nucleic acid assembly composition comprises an ionizable lipid.

88. The method of any one of claims 77-87, wherein the lipid nucleic acid assembly composition comprises an ionizable lipid and wherein the ionizable lipid has a pKa in the range of from about 5.1 to about 7.4, such as from about 5.5 to about 6.6, from about 5.6 to about 6.4, from about 5.8 to about 6.2, or from about 5.8 to about 6.5.

89. The method of any one of claims 77-88, wherein the lipid nucleic acid assembly composition comprises (i) an amine lipid; (ii) a helper lipid; (iii) a stealth lipid; (iv) a neutral lipid; or combinations of one or more of (i)-(iv).

90. The method of claim 89, wherein (i) the amine lipid is Lipid A; (ii) the helper lipid is cholesterol; (iii) the stealth lipid is PEG2k-DMG; (iv) the neutral lipid is DSPC; or combinations of one or more of (i)-(iv).

91. The method of any one of claims 77-90, wherein the N/P ratio of the lipid nucleic acid assembly composition is about 6.

92. The method of any one of claims 77-91, wherein the lipid nucleic acid assembly composition comprises about 50 mol-% amine lipid such as Lipid A; about 9 mol-% neutral lipid such as DSPC; about 3 mol-% of stealth lipid such as a PEG lipid, such as PEG2k-DMG, and the remainder of the lipid component is helper lipid such as cholesterol wherein the N/P ratio of the is about 6.

93. The method of any one of claims 77-92, wherein the lipid nucleic acid assembly composition comprises about 35 mol-% amine lipid such as Lipid A; about 15 mol-% neutral lipid such as DSPC; about 2.5 mol-% of stealth lipid such as a PEG lipid, such as PEG2k-DMG, and the remainder of the lipid component is helper lipid such as cholesterol wherein the N/P ratio of the is about 6.

94. The method of any one of claims 77-93, comprising one gRNA that targets a gene that reduces or eliminates MHC class I expression on the surface of a cell, and/or one gRNA that targets a gene that reduces or eliminates MHC class II expression on the surface of a cell, and/or one gRNA that targets a gene that reduces or eliminates endogenous TCR expression.

95. The method of any one of claims 77-94, comprising at least two gRNAs selected from: one gRNA that targets a gene that reduces or eliminates MHC class I expression on the surface of a cell, one gRNA that targets a gene that reduces or eliminates MHC class II expression on the surface of a cell, and one gRNA that targets a gene that reduces or eliminates endogenous TCR expression.

96. The method of any one of claims 77-95, comprising one gRNA that targets a gene that reduces or eliminates MHC class I expression on the surface of a cell, one gRNA that targets a gene that reduces or eliminates MHC class II expression on the surface of a cell, and one gRNA that targets a gene that reduces or eliminates endogenous TCR expression.

97. The method of any one of claims 77-96, comprising one gRNA selected from a gRNA that targets TRAC, TRBC, B2M, HLA-A, or CIITA.

98. The method of any one of claims 77-97, wherein the gRNA targets TRBC, wherein the gRNA comprises a guide sequence chosen from: i) SEQ ID NOs: 706-721; ii) at least 17, 18, 19, or 20 contiguous nucleotides of a sequence selected from SEQ ID NOs: 706-721; iii) a guide sequence at least 95%, 90%, or 85% identical to a sequence selected from SEQ ID NOs: 706-721; iv) a sequence that comprises 10 contiguous nucleotides±10 nucleotides of a genomic coordinate listed in Table 5A; v) at least 17, 18, 19, or 20 contiguous nucleotides of a sequence from (iv); or vi) a guide sequence that is at least 95%, 90%, or 85% identical to a sequence selected from (v).

99. The method of any one of claims 77-97, wherein the gRNA targets TRBC, wherein the gRNA comprises a guide sequence chosen from: i) SEQ ID NOs: 618-669; ii) at least 17, 18, 19, or 20 contiguous nucleotides of a sequence selected from SEQ ID NOs: 618-669; iii) a guide sequence at least 95%, 90%, or 85% identical to a sequence selected from SEQ ID NOs: 618-669; iv) a sequence that comprises 10 contiguous nucleotides±10 nucleotides of a genomic coordinate listed in Table 5B; v) at least 17, 18, 19, or 20 contiguous nucleotides of a sequence from (iv); or vi) a guide sequence that is at least 95%, 90%, or 85% identical to a sequence selected from (v).

100. The method of any one of claims 77-97, comprising at least two gRNAs selected from a gRNA that targets TRAC, TRBC, or B2M, wherein the two guide RNAs do not target the same gene.

101. The method of any one of claims 77-97, comprising at least two gRNAs selected from a gRNA that targets TRAC, TRBC, or HLA-A, wherein the two guide RNAs do not target the same gene.

102. The method of any one of claims 77-97, comprising one guide RNA that targets TRAC, and one gRNA that targets TRBC.

103. The method of any one of claims 77-97, comprising one guide RNA that targets B2M, and one gRNA that targets CIITA.

104. The method of any one of claims 77-97, comprising one guide RNA that targets HLA-A, and one gRNA that targets CIITA, optionally wherein the cell is homozygous for HLA-B and homozygous for HLA-C.

105. The method of any one of claims 77-97, comprising one guide RNA that targets TRAC, and one gRNA that targets TRBC, and one gRNA that targets B2M.

106. The method of any one of claims 77-97, comprising one guide RNA that targets TRAC, and one gRNA that targets TRBC, and one gRNA that targets HLA-A, optionally wherein the cell is homozygous for HLA-B and homozygous for HLA-C.

107. The method of any one of claims 77-97, comprising one guide RNA that targets TRAC, and one gRNA that targets TRBC, one gRNA that targets B2M, and one gRNA that targets CIITA.

108. The method of any one of claims 77-97, comprising one guide RNA that targets TRAC, and one gRNA that targets TRBC, one gRNA that targets HLA-A, and one gRNA that targets CIITA, optionally wherein the cell is homozygous for HLA-B and homozygous for HLA-C.

109. The method of any one of claims 5-10, 12-14, 19-67, and 77-108, wherein the method generates a cytosine (C) to thymine (T) conversion when present within a target sequence, optionally wherein if the nickase is a SpyCas9 nickase, the C to T conversion comprises 1-12 C to T conversions, and if the nickase is a NmeCas9 nickase, the C to T conversion comprises 1-20 C to T conversions.

110. The method of any one of claims 5-10, 12-14, 19-67, and 77-109, wherein the method causes at least 60% C-to-T conversion relative to the total edits in the target sequence.

111. The method of any one of claims 5-10, 12-14, 19-67, and 77-110, wherein the method causes at least 65%, 70%, 75%, 80%, 85%, 90%, 95%, 98%, or 99% C-to-T conversion relative to the total edits in the target sequence.

112. The method of any one of claims 5-10, 12-14, 19-67, and 77-111, wherein the ratio of C-to-T conversion to unintended edits is larger than 1:1.

113. The method of any one of claims 5-10, 12-14, 19-67, and 77-112, wherein the ratio of C-to-T conversion to unintended edits is from 2:1 to 99:1.

114. The method of any one of claims 5-10, 12-14, 19-67, and 77-113, wherein the ratio of C-to-T conversion to unintended edits is 2:1, 3:1, 4:1, 5:1, 6:1, 7:1, or 8:1.

115. The method of any one of claims 5-10, 12-14, 19-67, and 77-114, wherein the method causes the cytidine deaminase to make a base edit corresponding to any one of positions −1 to 10 relative to the 5′ end of the guide sequence.

116. The method of any one of claims 5-10, 12-14, 19-67, and 77-115, wherein the method causes the cytidine deaminase to make a base edit at a cytidine present at position 1, 2, 3, 4, 5, 6, 7, 8, 9, or 10 nucleotides from the 5′ end of the guide sequence.

117. The method of any one of claims 5-10, 12-14, 19-67, and 77-116, wherein the nickase is a SpyCas9 nickase, and the method causes the cytidine deaminase to make a base edit at a cytidine present at position 1, 2, 3, 4, 5, 6, 7, 8, 9, 10, or 11 nucleotides from the 5′ end of the guide sequence.

118. The method of any one of claims 5-10, 12-14, 19-67, and 77-116, wherein the nickase is a NmeCas9 nickase, and the method causes the cytidine deaminase to make a base edit at a cytidine present at position 4, 5, 6, 7, 8, 9, 10, 11, 12, 13, 14, 15, 16, 17, 18, 19, or 20 nucleotides from the 5′ end of the guide sequence.

119. The method of any one of claims 5-10, 12-14, 19-67, and 77-118, wherein the first mRNA, the second mRNA, and the guide RNA if present, delivered at a ratio of about 6:2:3 (w:w:w).

120. The method, cell, or engineered cell of any one of claims 5-10, 12-67, and 77-119, wherein the cell is a lymphocyte.

121. The method or use of any one of claims 5-10, 12-14, 19-67, and 75-120, wherein the modification of the target gene is in vivo.

122. The method or use of any one of claims 5-10, 12-14, 19-67, and 75-120, wherein the modification of the target gene is ex vivo.

123. The method or use of any one of claims 5-10, 12-14, 19-67, and 75-122, wherein the modification of the target gene reduces or eliminates expression of the target gene.

124. The method or use of any one of claims 5-10, 12-14, 19-67, and 75-123, wherein the genome editing or modification of the target gene reduces expression of the target gene by at least 50%, 55%, 60%, 65%, 70%, 75%, 80%, 85%, 90%, or 95%.

125. The method or use of any one of claims 5-10, 12-14, 19-67, and 75-124, wherein the genome editing or modification of the target gene produces a missense mutation in the gene.

126. A polypeptide comprising a cytidine deaminase and an RNA-guided nickase, wherein the polypeptide does not comprise a uracil glycosylase inhibitor (UGI).

127. A ribonucleoprotein complex (RNP) comprising the polypeptide of claim 126 and a guide RNA, wherein if the RNP comprises a SpyCas9 nickase, then the guide RNA is a Spy guide RNA, and wherein if the RNP comprises a NmeCas nickase, then the guide RNA is a Nme guide RNA.

128. A composition comprising a first polypeptide comprising a cytidine deaminase and an RNA-guided nickase, wherein the first polypeptide does not comprise a uracil glycosylase inhibitor (UGI), and a second polypeptide comprising a UGI, wherein the second polypeptide is different from the first polypeptide.

129. The polypeptide, RNP, or composition of any one of claims 126-128, wherein the cytidine deaminase is fused to the RNA-guided nickase via a peptide linker, optionally XTEN or a peptide linker comprising a sequence of GTKDSTKDIPETPSKD (SEQ ID NO: 268).

130. The polypeptide, RNP, or composition of any one of claims 126-128, wherein the cytidine deaminase is attached to a linker comprising an organic molecule, polymer, or chemical moiety.

131. A pharmaceutical composition comprising the mRNA, RNP, composition, or polypeptide of claims 1-4, 8-11, 19-69, 74, and 126-130 and a pharmaceutically acceptable carrier.

132. A kit comprising the mRNA, RNP, composition, or polypeptide of any of 1-4, 8-11, 19-69, 74, and 126-130.

133. The mRNA, RNP, composition, method, use, cell, or engineered cell of any one of 1-132, wherein the polypeptide comprising a cytidine deaminase and an RNA-guided nickase includes: the cytidine deaminase, a linker, and the RNA-guided nickase in amino to carboxy terminal order.

134. A method of altering a DNA sequence within a TRAC gene, comprising delivering to a cell:
a. a gRNA comprising a guide sequence chosen from: i) SEQ ID NOs: 706-721; ii) at least 17, 18, 19, or 20 contiguous nucleotides of a sequence selected from SEQ ID NOs: 706-721; iii) a guide sequence at least 95%, 90%, or 85% identical to a sequence selected from SEQ ID NOs: 706-721; iv) a sequence that comprises 10 contiguous nucleotides±10 nucleotides of a genomic coordinate listed in Table 5A; v) at least 17, 18, 19, or 20 contiguous nucleotides of a sequence from (iv); or vi) a guide sequence that is at least 95%, 90%, or 85% identical to a sequence selected from (v); or
b. a nucleic acid encoding a gRNA of (a.).

135. A method of reducing the expression of a TRAC gene, comprising delivering to a cell:
a. a gRNA comprising a guide sequence chosen from: i) SEQ ID NOs: 706-721; ii) at least 17, 18, 19, or 20 contiguous nucleotides of a sequence selected from SEQ ID NOs: 706-721; iii) a guide sequence at least 95%, 90%, or 85% identical to a sequence selected from SEQ ID NOs: 706-721; iv) a sequence that comprises 10 contiguous nucleotides±10 nucleotides of a genomic coordinate listed in Table 5A; v) at least 17, 18, 19, or 20 contiguous nucleotides of a sequence from (iv); or vi) a guide sequence that is at least 95%, 90%, or 85% identical to a sequence selected from (v); or
b. a nucleic acid encoding a gRNA of (a.).

136. A method of immunotherapy comprising administering a composition comprising an engineered cell to a subject,
wherein the cell comprises a genomic modification of at least one nucleotide within the genomic coordinates selected from:
chr14: 22547596-22547616; chr14: 22550570-22550590; chr14: 22547763-22547783; chr14: 22550596-22550616; chr14: 22550566-22550586; chr14: 22547753-22547773; chr14: 22550601-22550621; chr14: 22550599-22550619; chr14: 22547583-22547603; chr14: 22547671-22547691; chr14: 22547770-22547790; chr14: 22547676-22547696; chr14: 22547772-22547792; chr14: 22547771-22547791; chr14: 22547733-22547753; chr14: 22547776-22547796; or
wherein the cell is engineered by delivering to the cell:
a. a gRNA comprising a guide sequence chosen from: i) SEQ ID NOs: 706-721; ii) at least 17, 18, 19, or 20 contiguous nucleotides of a sequence selected from SEQ ID NOs: 706-721; iii) a guide sequence at least 95%, 90%, or 85% identical to a sequence selected from SEQ ID NOs: 706-721; iv) a sequence that comprises 10 contiguous nucleotides±10 nucleotides of a genomic coordinate listed in Table 5A; v) at least 17, 18, 19, or 20 contiguous nucleotides of a sequence from (iv); or vi) a guide sequence that is at least 95%, 90%, or 85% identical to a sequence selected from (v); or
b. a nucleic acid encoding a gRNA of (a.).

137. A method of altering a DNA sequence within a TRBC1 and/or TRBC2 gene, comprising delivering a composition to a cell, wherein the composition comprises:
a. a gRNA comprising a guide sequence chosen from: i) SEQ ID NOs: 618-669; ii) at least 17, 18, 19, or 20 contiguous nucleotides of a sequence selected from SEQ ID NOs: 618-669; iii) a guide sequence at least 95%, 90%, or 85% identical to a sequence selected from SEQ ID NOs: 618-669; iv) a sequence that comprises 10 contiguous nucleotides±10 nucleotides of a genomic coordinate listed in Table 5B; v) at least 17, 18, 19, or 20 contiguous nucleotides of a sequence from (iv); or vi) a guide sequence that is at least 95%, 90%, or 85% identical to a sequence selected from (v); or
b. a nucleic acid encoding a guide RNA of (a.).

138. A method of reducing the expression of a TRBC1 and/or TRBC2 gene, comprising delivering a composition to a cell, wherein the composition comprises:
a. a gRNA comprising a guide sequence chosen from: i) SEQ ID NOs: 618-669; ii) at least 17, 18, 19, or 20 contiguous nucleotides of a sequence selected from SEQ ID NOs: 618-669; iii) a guide sequence at least 95%, 90%, or 85% identical to a sequence selected from SEQ ID NOs: 618-669; iv) a sequence that comprises 10 contiguous nucleotides±10 nucleotides of a genomic coordinate listed in Table 5B; v) at least 17, 18, 19, or 20 contiguous nucleotides of a sequence from (iv); or vi) a guide sequence that is at least 95%, 90%, or 85% identical to a sequence selected from (v); or
b. a nucleic acid encoding a guide RNA of (a.).

139. A method of immunotherapy comprising administering a composition comprising an engineered cell to a subject,
wherein the cell comprises a modification of at least one nucleotide within the genomic coordinates selected from: chr7: 142791757-142791777; chr7: 142801104-142801124; chr7: 142791811-142791831; chr7: 142801158-142801178; chr7: 142792728-142792748; chr7: 142791719-142791739; chr7: 142791766-142791786; chr7: 142801113-142801133; chr7: 142791928-142791948; chr7: 142801275-142801295; chr7: 142792062-142792082; chr7: 142801409-142801429; chr7: 142792713-142792733; chr7: 142802126-142802146; chr7: 142791808-142791828; chr7: 142801155-142801175; chr7: 142792003-142792023; chr7: 142801350-142801370; chr7: 142791760-142791780; chr7: 142791715-142791735; chr7: 142792781-142792801; chr7: 142792040-142792060; chr7: 142801387-142801407; chr7: 142791862-142791882; chr7: 142791716-142791736; chr7: 142791787-142791807; chr7: 142791759-142791779; chr7: 142801106-142801126; chr7: 142791807-142791827; chr7: 142801154-142801174; chr7: 142791879-142791899; chr7: 142801226-142801246; chr7: 142791805-142791825; chr7: 142791700-142791720; chr7: 142791765-142791785; chr7: 142801112-142801132; chr7: 142791820-142791840; chr7: 142791872-142791892; chr7: 142801219-142801239; chr7: 142791700-142791720; chr7: 142791806-142791826; chr7: 142801153-142801173; chr7: 142792035-142792055; chr7: 142792724-142792744; chr7: 142792754-142792774; chr7: 142791804-142791824; chr7: 142792684-142792704; chr7: 142791823-142791843; chr7: 142792728-142792748; chr7: 142792721-142792741; chr7: 142792749-142792769; chr7: 142792685-142792705; chr7: 142791816-142791836; chr7: 142801163-142801183; chr7: 142792686-142792706; chr7: 142791793-142791813; chr7: 142793110-142793130; chr7: 142791815-142791835; chr7: 142801162-142801182; chr7: 142792770-142792790; chr7: 142792047-142792067; chr7: 142801394-142801414; chr7: 142791871-142791891; chr7: 142801218-142801238; chr7: 142791894-142791914; chr7: 142792723-142792743; chr7: 142792724-142792744; chr7: 142791897-142791917; chr7: 142801244-142801264; chr7: 142792757-142792777; chr7: 142792740-142792760; chr7: 142792758-142792778; or
wherein the cell is engineered by delivering to a cell:
a. a gRNA comprising a guide sequence chosen from: i) SEQ ID NOs: 618-669; ii) at least 17, 18, 19, or 20 contiguous nucleotides of a sequence selected from SEQ ID NOs: 618-669; iii) a guide sequence at least 95%, 90%, or 85% identical to a sequence selected from SEQ ID NOs: 618-669; iv) a sequence that comprises 10 contiguous nucleotides±10 nucleotides of a genomic coordinate listed in Table 5B; v) at least 17, 18, 19, or 20 contiguous nucleotides of a sequence from (iv); or vi) a guide sequence that is at least 95%, 90%, or 85% identical to a sequence selected from (v); or
b. a nucleic acid encoding a guide RNA of (a.).

140. A composition comprising:
a. a gRNA comprising a guide sequence chosen from: i) SEQ ID NOs: 706-721; ii) at least 17, 18, 19, or 20 contiguous nucleotides of a sequence selected from SEQ ID NOs: 706-721; iii) a guide sequence at least 95%, 90%, or 85% identical to a sequence selected from SEQ ID NOs: 706-721; iv) a sequence that comprises 10 contiguous nucleotides±10 nucleotides of a genomic coordinate listed in Table 5A; v) at least 17, 18, 19, or 20 contiguous nucleotides of a sequence from (iv); or vi) a guide sequence that is at least 95%, 90%, or 85% identical to a sequence selected from (v); and optionally
b. the mRNA or composition of any one of the preceding claims relating to mRNA or compositions.

141. A composition comprising:
a. a gRNA comprising a guide sequence chosen from: i) SEQ ID NOs: 618-669; ii) at least 17, 18, 19, or 20 contiguous nucleotides of a sequence selected from SEQ ID NOs: 618-669; iii) a guide sequence at least 95%, 90%, or 85% identical to a sequence selected from SEQ ID NOs: 618-669; iv) a sequence that comprises 10 contiguous nucleotides±10 nucleotides of a genomic coordinate listed in Table 5B; v) at least 17, 18, 19, or 20 contiguous nucleotides of a sequence from (iv); or vi) a guide sequence that is at least 95%, 90%, or 85% identical to a sequence selected from (v); and optionally
b. the mRNA or composition of any one of the preceding claims relating to mRNA or compositions.

142. An engineered cell which has reduced or eliminated surface expression of TRAC, comprising a genetic modification in a human TRAC gene, wherein the genetic modification comprises a modification of at least one nucleotide within the genomic coordinates selected from:
chr14: 22547596-22547616; chr14: 22550570-22550590; chr14: 22547763-22547783; chr14: 22550596-22550616; chr14: 22550566-22550586; chr14: 22547753-22547773; chr14: 22550601-22550621; chr14: 22550599-22550619; chr14: 22547583-22547603; chr14: 22547671-22547691; chr14: 22547770-22547790; chr14: 22547676-22547696; chr14: 22547772-22547792; chr14: 22547771-22547791; chr14: 22547733-22547753; chr14: 22547776-22547796.

143. An engineered cell which has reduced or eliminated surface expression of TRBC1/2, comprising a genetic modification in a human TRBC1/2 gene, wherein the genetic modification comprises a modification of at least one nucleotide within the genomic coordinates selected from:
chr7: 142791757-142791777; chr7: 142801104-142801124; chr7: 142791811-142791831; chr7: 142801158-142801178; chr7: 142792728-142792748; chr7: 142791719-142791739; chr7: 142791766-142791786; chr7: 142801113-142801133; chr7: 142791928-142791948; chr7: 142801275-142801295; chr7: 142792062-142792082; chr7: 142801409-142801429; chr7: 142792713-142792733; chr7: 142802126-142802146; chr7: 142791808-142791828; chr7: 142801155-142801175; chr7: 142792003-142792023; chr7: 142801350-142801370; chr7: 142791760-142791780; chr7: 142791715-142791735; chr7: 142792781-142792801; chr7: 142792040-142792060; chr7: 142801387-142801407; chr7: 142791862-142791882; chr7: 142791716-142791736; chr7: 142791787-142791807; chr7: 142791759-142791779; chr7: 142801106-142801126; chr7: 142791807-142791827; chr7: 142801154-142801174; chr7: 142791879-142791899; chr7: 142801226-142801246; chr7: 142791805-142791825; chr7: 142791700-142791720; chr7: 142791765-142791785; chr7: 142801112-142801132; chr7: 142791820-142791840; chr7: 142791872-142791892; chr7: 142801219-142801239; chr7: 142791700-142791720; chr7: 142791806-142791826; chr7: 142801153-142801173; chr7: 142792035-142792055; chr7: 142792724-142792744; chr7: 142792754-142792774; chr7: 142791804-142791824; chr7: 142792684-142792704; chr7: 142791823-142791843; chr7: 142792728-142792748; chr7: 142792721-142792741; chr7: 142792749-142792769; chr7: 142792685-142792705; chr7: 142791816-142791836; chr7: 142801163-142801183; chr7: 142792686-142792706; chr7: 142791793-142791813; chr7: 142793110-142793130; chr7: 142791815-142791835; chr7: 142801162-142801182; chr7: 142792770-142792790; chr7: 142792047-142792067; chr7: 142801394-142801414; chr7: 142791871-142791891; chr7: 142801218-142801238; chr7: 142791894-142791914; chr7: 142792723-142792743; chr7: 142792724-142792744; chr7: 142791897-142791917; chr7: 142801244-142801264; chr7: 142792757-142792777; chr7: 142792740-142792760; chr7: 142792758-142792778.

144. A lipid nucleic acid assembly composition comprising an mRNA comprising an open reading frame encoding a polypeptide comprising a cytidine deaminase and an RNA-guided nickase, wherein the polypeptide does not comprise a uracil glycosylase inhibitor (UGI).

145. One or more lipid nucleic acid assembly compositions, optionally lipid nanoparticles, comprising:
(a) a first mRNA comprising a first open reading frame encoding a polypeptide comprising a cytidine deaminase and an RNA-guided nickase;
(b) a second mRNA comprising a second open reading frame encoding a uracil glycosylase inhibitor (UGI); and
(c) one or more guide RNAs.

146. The method, cell, or engineered cell of any one of claims 134-143, wherein the cell is an immune cell, a lymphocyte, or a T cell.
